(19) United States
(12) Patent Application Publication
(10) Pub. No.: US 2003/0088058 A1
(43) Pub. Date: May 8, 2003

(54) ISOPENICILLIN N SYNTHETASE AND DEACETOXYCEPHALOSPORIN C SYNTHETASE ENZYMES AND METHODS

(76) Inventors: CHRISTOPHER J SCHOFIELD, OXFORD (GB); JACK E BALDWIN, OXFORD (GB); IAN CLIFTON, OXFORD (GB); JANOS HAJDU, UPPSALA (SE); CHARLES M. H. HENSGENS, GRONINGEN (NL); PETER L ROACH, OXFORD (GB)

Correspondence Address: NIXON & VANDERHYE, 1100 NORTH GLEBE ROAD, 8TH FLOOR, ARLINGTON, VA, 222014714

(21) Appl. No.: 09/284,640

(22) PCT Filed: Oct. 15, 1997
(86) PCT No.: PCT/GB97/02838

(30) Foreign Application Priority Data
Oct. 15, 1996 (GB) 9621486.1

(51) Int. Cl. C07K 1/00; C12Q 1/00

(52) U.S. Cl. 530/350; 435/4

(57) ABSTRACT

A three-dimensional structure is described of a complex of isopenicillin N synthase (IPNS) with Fe and its substrate ACV. This structure is used to design modified enzymes IPNS, DAOCS, DACS, DAOC/DACS and other related enzymes of the penicillin and cephalosporin biosynthesis pathway, which modified enzymes may accept unnatural substrates or improve production efficiency or produce improved products. Specific modifications of specific amino acid residues are proposed and exemplified.

### INTRODUCTION

All commercially used penicillin and cephalosporin antibiotics and their derivatives are produced from fermentation derived materials containing a β-lactam ring. A range of organisms, including both prokaryotes and eukaryotes, and conditions may be used for their fermentation. Some are produced directly by fermentation followed by isolation. Others are produced by modification of materials produced by fermentation. Commercially used cephalosporins (also known as cephems) may be produced by modification of either fermentation derived penicillins or cephalosporins.

The biosynthetic pathway to the penicillins and cephalosporins has been extensively studied and involves the following steps (Scheme 1):

1. Three amino acids (L-α-aminoadipic acid, L-cysteine, L-valine) are condensed to form a tripeptide: L-δ-α-aminoadipoyl-L-cysteinyl-D-valine (ACV). During this process the L-valinyl residue is converted to a D-valinyl residue. This process is catalysed in vivo by the enzyme ACV synthetase and is common to both penicillin and cephalosporin biosynthesis.

2. ACV is converted to isopenicillin N in a step catalysed by the enzyme isopenicillin N synthase (IPNS). This step is common to both penicillin and cephalosporin biosynthesis.

3. In some organisms (e.g. Penicillium chrysogenum and Aspergillus nidulans

) isopenicillin N is converted by exchange of its L-δ-α-aminoadipoyl side chain to penicillins with other side chains, which are normally more hydrophobic than the side chain of isopenicillin N. This conversion may be catalysed by an amidohydrolase/acyltransferase enzyme. Examples of penicillins produced by this biosynthetic process include penicillin G (which has a phenylacetyl side chain) and penicillin V (which has a phenoxyacetyl side chain). These hydrophobic penicillins may be commercially produced by fermentation under the appropriate conditions.

4. In some organisms (e.g. Streptomyces clavuligerus and Cephalosporium acremonium) isopenicillin N is epimerised to penicillin N. This reaction is catalysed by an epimerase enzyme.

5. In some organisms (e.g. S. clavuligerus and C. acremonium) penicillin N is converted to deacetoxycephalosporin C (DAOC). This reaction is catalysed by deacetoxycephalosporin C synthase (DAOCS) in some organisms (e.g. Streptomyces clavuligerus) and by deacetoxy/deacetylcephalosporin C synthase (DAOC/DACS) in others (e.g. C. acremonium).

6. In some organisms (e.g. S. clavuligerus and C. acremonium) DAOC is converted to deacetylcephalosporin C (DAC). This reaction is catalysed by deacetylcephalosporin C synthase (DACS) in some organisms (e.g. S. clavuligerus) and by deacetoxy/deacetylcephalosporin C synthase (DAOC/DACS) in others (e.g. C. acremonium).

Further biosynthetic steps to give other cephalosporin derivatives may also occur, e.g. in C. acremonium DAC may be converted to cephalosporin C and in Streptomyces spp. DAC may be converted to cephamycin C. The genes encoding for each of the enzymes catalysing steps 1-6 above have been identified and sequenced.

Fermented penicillins, cephalosporins, their biosynthetic intermediates, and their derivatives may be of use as antibiotics or as intermediates in the production of antibiotics. Penicillins with hydrophobic side chains may be used for the preparation of cephalosporins or intermediates used in the preparation of cephalosporins, e.g. penicillins (including, but not exclusively, penicillin G and penicillin V) may be used to prepare C-3 exomethylene cephams which may be used as intermediates in the preparation of the commercial antibiotics, e.g. Cefachlor (Scheme 2).

For reviews see J. E. Baldwin and C. J. Schofield, in ‘The Chemistry of β-lactams (Ed. M. I. Page), Chapter 1, Blackie, London 1992; Aharonowitz et al, Ann. Rev. Microbiol., 1992, 46, 461; Cooper, Bioorg. Med. Chem., 1993, 1, 1; Baldwin and Abraham, Nat. Prod. Report., 1989, 5, 129; Baldwin, J. Heterocyclic. Chem., 1990, 27, 91.

### SUMMARY OF INVENTION

This invention is based on our determination of the three-dimensional structure of IPNS. That the structure of IPNS complexed to manganese has been determined, was reported by some of us in Nature, Volume 375, Jun. 22, 1995, pages 700-704. That publication did not include the co-ordinates of the individual amino acid residues, and these are now provided. Scheme 2 of that paper contains the amino acid sequence of IPNS, and also DACS, DAOCS and DAOC/DACS and other structurally related enzymes, each of which is published in Swissprot or Genbank or other database.

We have now determined the structure of a complex of IPNS with Fe and ACV which is a substrate for the enzyme (see Scheme 1). In solution it is this complex, and not the IPNS-Mn complex, that is actually formed during step 2 of the biosynthesis of bicyclic β-lactams. Because the amino acid sequences of DAOCS, DAOC/DACS, DACS and other oxidases and oxygenases are so similar to that of IPNS, it is reasonable to expect that the structures of those enzymes are at least similar to that of IPNS.

We have also determined the structures of complexes of IPNS with Fe and with various analogues of ACV (in which another amino acid replaced L-valine), specifically AC glycine, AC aminobutyrate, AC alanine and AC proparglyglycine. These structures have been determined in the absence and in the presence of nitrous oxide NO. Exposure of these complexes to dioxygen alters the structures, and these altered structures have also been determined by us. From information given herein about the IPNS-Fe-ACV complex, a skilled reader is able to make and characterise the other complexes referred to in this paragraph, so structural details of those other complexes are not given herein.

Thus in one aspect the invention provides Isopenicillin N synthase (IPNS) in the form of: a complex with Mn having a structure designated by the X-ray co-ordinates in Table 2; or a complex with Fe and its substrate, said complex having a structure designated by the X-ray co-ordinates in Table 3.

In another aspect the invention provides Isopenicillin N synthase (IPNS) in the form of: a complex with Fe and an analogue of its substrate, either in the absence or in the presence of nitrous oxide or dioxygen, said complex having a structure designated by X-ray co-ordinates analogous to that set out in Table 3.

An analogue of an IPNS substrate is a substrate oxidised by IPNS to give preferably (but not exclusively) a bicyclic compound containing a β-lactam ring.

Table 2 sets out co-ordinates of individual amino acid residues in a crystalline complex of IPNS with manganese.

Table 3 sets out co-ordinates of individual amino acid residues in a crystalline complex of IPNS with Fe and ACV.

Knowledge, derived from the X-ray co-ordinates, of the three-dimensional structures of this family of related enzymes permits a skilled worker to identify specific amino acids that might be changed in order to alter or improve the properties of the enzyme in some way. While it is not possible from 3D structural information alone to predict that a specific amino acid mutation will produce a specific change in the properties of the enzyme, it is possible to identify a rather small number of amino acid residues where modification may be expected to change/improve the properties of the enzyme. The problem of identifying useful amino acid mutations is thus reduced to a level where it can readily be tackled by routine screening procedures.

Thus in one aspect the invention provides use of the three dimensional structure of a first enzyme selected from IPNS, DAOCS, DACS, DAOCS/DACS and other related enzymes of the penicillin and cephalosporin biosynthesis pathway, for the modification of a second selected from IPNS, DAOCS, DACS, DAOCS/DACS and other related enzymes of the penicillin and cephalosporin biosynthesis pathway.

The three dimensional structure of a first enzyme may be the three dimensional structure of the IPNS-Fe-substrate complex referred to above. It may, however, also be that of DAOCS, DACS, DAOC/DACS or another oxygenase/oxidase related by sequence or structure (e.g. 1-aminocylopropane-1-carboxylic acid oxidase) to any of IPNS, DAOCS, DACS or DAOC/DACS. The structure of the IPNS-Fe-ACV complex may be derived from two or more crystalline polymorphs, all of which are envisaged. The structure may alternatively be of the enzyme in free form or in the form of some other complex such as with Mn, or with other Fe or ACV analogues, or enzyme inhibitors, or other enzyme modifiers. Preferably the second enzyme is the same as the first enzyme e.g. the 3D structure of IPNS is used as a basis for modifying IPNS. Alternatively the 3D structure of one first enzyme may be used as a basis for modifying a second structurally related enzyme.

Central to the elucidation of the structure of crystalline proteins is the discovery of conditions for the production of crystals with diffract X-rays to a sufficiently high resolution. Since the cofactors (e.g. Fe(II)) and substrates (e.g. ACV) of the family of enzymes to which IPNS, DAOCS, DACS, etc. belong are sensitive to modification by reaction with dioxygen, the crystallisation of these enzymes is preferably carried out under an anaerobic atmosphere or one containing only a very low concentration of dioxygen.

The modified enzyme(s) may be used in vitro or introduced via recombinant molecular biology techniques into an organism so that new materials can be fermented. It is recognised that multiple modifications may have to be made to an enzyme in order to change its substrate/product selectivity, and/or improve it efficiency. It is recognised that more than one modified enzyme may be used to effect the desired transformation. It is recognised that in order to change the nature of the enzyme-substrate/intermediate/product interactions at a particular enzyme-substrate/intermediate interface modifications may be made to the enzyme either immediately at the interface or away from it. It is recognised that the modifications may result in hybrid enzymes containing sequences from, e.g. IPNS and DAOCS or IPNS and DACS or any combination of IPNS, DAOCS, DACS or DAOCS/DACS or other related enzymes. It is also recognised that it may be desirable to further modify the organism in which the modified enzyme is to be introduced, e.g. by blocking a particular pathway in that organism (using the techniques of molecular biology) in order to modify flux through the desired/modified pathway, by introducing other enzyme activities, or by other modifications. The organism into which the modified enzyme will be used may or may not contain parts of the penicillin and cephalosporin biosynthetic machinery. The organism may already have been modified to optimise or minimise production of particular products or consumption of particular nutrients. More than one modified enzyme may be used in conjunction either in vitro or in vivo in an organism for the production of desirable products.

While modifications for numerous specific purposes are discussed below, it is possible to say in general that useful modifications will be of three kinds:

Those which permit the enzyme to accept unnatural substrates [i.e. substrates not normally present in the organism (which may or may not be an organism in which the enzyme is naturally occurring) in which the enzyme is operating] for the preparation of new or commercially valuable anti-bacterial materials or intermediates for the production of pharmaceutical products;

Those which enable the enzyme to produce unnatural products [i.e. products not naturally produced in the organism in which the enzyme is operating, including 3-exomethylene cephams and cephalosporins with hydrophobic side chains at the C-7 position such as phenylacetamido or phenoxyacetamido, or other unnatural side chains such as adipoyl] or improve the production of natural products of commercial value.

Those which enhance the ability of the enzyme to produce useful products. For example DAOCS is known to catalyse the production of phenylacetylcephalosporin C from penicillin G (Baldwin et al., Proceedings of the 7^{th} International Symposium on the genetics of Industrial Microorganisms, Abstract, p 262, 1994). However, this conversion is much less efficient than the DAOCS catalysed conversion of penicillin N to DAOC. Modifications made to DAOCS may increase the efficiency of its catalytic conversion to penicillin G.

In another aspect this invention provides modified enzymes that result from application of the aforementioned techniques. These are enzymes having significant (as defined below) sequence and thus structural similarity with IPNS. Thus, structures of these enzymes may be predicted on the basis of the IPNS structures. Preferably there will be sequence similarity/identity between most of the modified enzyme and a major part of IPNS. Previous sequence comparisons (Roach et al., Nature, 1995, 375, 700), using pairwise comparisons of the sequences followed by single linkage cluster analysis show that IPNS, DAOCS, DACS and DAOC/DACS cluster with standard deviations scores of >5.0 (Barton and Sternberg, J. Mol. Biol., 1987, 198, 327). Scores over 5.0 and preferably over 6.0 indicate that the sequence alignments will be correct within all or most of the protein secondary structural elements (Barton, Methods in Enzymol., 1990, 183, 403); thus they have significantly similar sequences and hence structures. Note there are other criteria which may be used to ascertain significant sequence similarity for example % identity or % similarity of amino acids possessing side chains with similar physico-chemical properties (Barton and Sternberg, J. Mol. Biol., 1987, 198, 327). Thus, on the basis of sequence comparisons it is possible to predict the structure of one enzyme (e.g. DAOCS, DACS or DAOC/DACS) from another (e.g. IPNS). Further, it is recognised that although two enzymes may have structures in which secondary structural elements are largely or wholly conserved, differences in the structures of the two enzymes may result from the side chains of the amino acids forming the secondary structural elements. These differences, which may alter the substrate/product selectivities of the compared enzymes, may be predictable if the three dimensional structure of one of the enzymes is known. An example: the natural substrate for IPNS, ACV, has an L-configured aminoadipoyl side chain, whereas the substrates for DAOCS, DACS and DAOC/DACS, i.e. penicillin N and DAOC, have D-configured aminoadipoyl side chains. This difference in selectivity may result from the different arrangement of amino acid side chain binding sites between IPNS and DAOCS, DACS, and DAOC/DACS, Further, it is recognised that there may be significant variations between enzymes which show significant sequence/structural similarity (i.e. with standard deviation scores >5.0) in exterior regions of the enzymes, e.g. in loops and at the N- and C- termini. The relative importance of these regions in substrate binding may be predicted by comparison with a known crystal structure of an enzyme with significant sequence similarity.

In one aspect, at least one of the following amino acid residues is modified:

N287; R87; A88; Y189; S183; Y91; F285; Q330; T331; V185; L106; C104; V217; L324, L317; I325; L321; S210.

The residue numbering herein is taken from the paper Nature, 1995, 376, 700-704 referred to above, i.e. is that shown for A. nidulans in SwinProt PO 5326. These modifications are expected to have an effect on side chain substituents at the 6-position of the penicillin molecule, or the 7-position of the cephalosporin molecule. In each case, the stated amino acid residue may be replaced by the residue of any other amino acid. But in order to change the selectivity of the enzyme to accept substrates with hydrophilic or neutral side chains, the replacement is preferably to make the side chain binding pocket more hydrophobic.

In another aspect at least one of the following amino acid is residues is modified:

V272, L231; L223; P283; T221; F211; F285; Q330; I187; V185; Y189; R279, S281; N230; Q225; N252; S210.

These modifications are expected to be associated with changes in the ring structure of the penicillin/cephalosporin molecule.

There follow examples of specific changes envisaged as a result of these modifications.

a) The structure of IPNS is modified in its active site region to accept unnatural substrates to produce penicillins or other bicyclic β-lactams of commercial use with hydrophobic side chains (Scheme 5). The process may include the following modifications (other modifications based on the use of the crystal structure of IPNS are not excluded):

Note, R87F/A/G/V/L/I/T/W/M/C/N/Q/P/S/T/E/D/R/K/H indicates that residue arginine-87, using the Aspergillus nidulans IPNS numbering scheme is modified to phenylalanine or alanine etc. See Roach et al Nature, 1995, 3757 700-704.)

N287F/A/G/V/L/I/W/M/C/N/Q/P/S/E/D/K/H/Y
R87F/A/G/V/L/I/W/M/C/N/Q/P/S/E/D/K/H/Y
Y189F/A/G/V/L/I/W/M/C/N/Q/P/S/E/D/K/H/R
S183F/A/G/V/L/I/W/M/C/N/Q/P/T/E/D/K/H/R/Y
Y91F/A/G/V/L/I/W/M/C/N/Q/P/S/T/E/D/K/H/R
F285A/G/V/L/I/W/M/C/N/Q/P/S/T/E/D/R/K/H/Y
Q330F/A/G/V/L/I/W/M/C/N/P/S/T/E/D/R/K/H/Y
T331F/A/G/V/L/I/W/M/C/N/P/S/E/D/R/K/H/Q/Y
V185F/A/G/L/I/W/T/M/C/N/P/S/E/D/R/K/H/Q/Y
L106F/A/G/V/I/T/W/M/C/N/P/S/E/D/R/K/H/Q/Y
C104F/A/G/V/L/I/T/W/M/N/P/S/E/D/R/K/H/Q/Y
V217F/A/G/L/I/T/W/M/C/N/P/S/E/D/R/K/H/Q/Y
L324F/A/G/I/L/T/W/M/C/N/P/S/E/D/R/K/H/Q/Y
L317F/A/G/V/I/T/W/M/C/N/P/S/E/D/R/K/H/Q/Y
I325F/A/G/V/L/T/W/M/C/N/P/S/E/D/R/K/H/Q/Y
L321F/A/G/V/I/T/W/M/C/N/P/S/E/D/R/K/H/Q/Y
S210F/A/G/V/I/L/T/W/M/C/N/P/E/D/R/K/H/Q/Y

Note in these and in subsequently proposed modifications the amino acid residue numbering scheme is based upon that used for A. nidulans IPNS aid the sequence alignments in Roach et al Nature, 1995, 375, 700-704, e.g. arginine-87 in IPNS remains named as arginine-87 for other aligned enzymes.

It is recognised that modifications to the side chain binding interactions and the valinyl binding interactions of IPNS may have to be made in conjunction with each other or with other modifications in order to produce a useful catalyst with the desired properties. Other modifications based on the use of the three dimensional structures of IPNS, DACS, DAOCS, DAOCS/DACS, other sequence related enzymes or complexes of these enzymes to their substrates, intermediates, modifiers, products or inhibitors are not excluded.

b) The structure of IPNS is modified in its active site region to accept natural or unnatural substrates to produce bicyclic β-lactams other than penicillins of commercial use (Scheme 6). For example the region of IPNS interacting with the valinyl residue of ACV may be modified such that IPNS produces 3-exomethylenecephams from ACV or other substrates for IPNS. The process may include the following modifications.

V272F/A/G/I/L/W/M/N/C/N/P/S/T/E/D/R/K/H/Q/Y
L231F/A/G/V/I/W/M/C/N/P/S/T/E/D/R/K/H/Q/Y
L223F/A/G/V/I/W/M/C/N/P/S/T/E/D/R/K/H/Q/Y
P283F/A/G/V/I/L/W/M/C/N/S/T/E/D/R/K/H/Q/Y
T221F/A/G/V/I/L/W/M/C/N/P/S/E/D/R/K/H/Q/Y
F211A/G/V/I/L/W/M/C/N/P/S/T/E/D/R/K/H/Q/Y
F285A/G/V/I/L/W/M/C/N/P/S/T/E/D/R/K/H/Q/Y
Q330F/A/G/V/I/L/W/M/C/N/P/S/T/E/D/R/K/H/Y
I187F/A/G/L/W/T/M/C/N/P/S/T/E/D/R/K/H/Q/Y
V185F/A/G/I/L/W/M/C/N/P/S/T/E/D/R/K/H/Q/Y
Y189F/A/G/V/I/L/W/M/C/N/P/S/T/E/D/R/K/H/Q
R279F/A/G/V/I/L/W/M/C/N/P/S/T/E/D/K/H/Q/Y
S281F/A/G/V/I/L/W/M/C/N/P/T/E/D/R/K/H/Q/Y
N230F/A/G/V/I/L/W/M/C/N/P/S/T/E/D/R/K/H/Q/Y
Q225F/A/G/V/I/L/W/M/C/N/P/S/E/D/R/K/H/Q/Y
N252F/A/G/V/I/L/W/M/C/N/P/S/E/D/R/K/H/Q/Y
S210F/A/G/V/I/L/T/W/M/C/N/P/E/D/R/K/H/Q/Y

It is recognised that modifications may have to be made in conjunction with each other or with other modifications to IPNS in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

c) The side chain binding interactions of IPNS are modified such that 6-aminopenicillins or other bicylic β-lactams may be produced in vitro or in vivo from dipeptides, such as cysteinyl-valine or other dipeptides (Scheme 7). Dipeptides may be produced (either in vitro or in vivo) by the use of a peptide synthetase enzyme, such as ACV synthetase (which may be modified by mutagenesis or other techniques to optimise dipeptide production) or by chemical synthesis. The process may include the following modifications:

N287F/A/G/V/L/I/W/M/C/N/Q/P/S/E/D/K/H/Y
R87F/A/G/V/L/I/W/M/C/N/Q/P/S/E/D/K/H/Y
Y189F/A/G/V/L/I/W/M/C/N/Q/P/S/T/E/D/K/H/R
S183F/A/G/V/L/I/W/M/C/N/Q/P/T/E/D/D/K/H/R/Y
Y91F/A/G/V/L/I/W/M/C/N/Q/P/S/T/E/D/K/H/R
F285A/G/V/L/I/W/M/C/N/Q/P/S/T/E/D/R/K/H/Y
Q330F/A/G/V/L/I/W/M/C/N/P/S/T/E/D/R/K/H/Y
T331F/A/G/V/L/I/W/M/V/N/P/S/E/D/R/K/H/Q/Y
V185F/A/G/L/I/W/M/C/N/P/S/T/E/D/R/K/H/Q/Y
L106F/A/G/V/I/W/M/C/N/P/S/T/E/D/R/K/H/Q/Y
C104F/A/G/V/L/I/W/M/N/P/S/T/E/D/R/K/H/Q/Y
V217F/A/G/L/I/W/M/C/N/P/S/T/E/D/R/K/H/Q/Y
L324F/A/G/L/I/W/M/C/N/P/S/T/E/D/R/K/H/Q/Y
L317F/A/G/V/I/W/M/C/N/P/S/T/E/D/R/K/H/Q/Y
I325F/A/G/V/L/W/M/C/N/P/S/T/E/D/R/K/H/Q/Y
L321F/A/G/V/I/W/M/C/N/P/S/T/E/D/R/K/H/Q/Y
S210F/A/G/V/I/L/T/W/M/C/N/P/E/D/R/K/H/Q/Y

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

d) The side chain binding interactions of IPNS are modified such that penams without any substituent at the 6-position or other bicylic β-lactams, without any substituent at the 6-position, may be produced in vitro or in vivo from dipeptides or amide substrates, such as 3-mercaptopropionyl-valine or other dipeptides or amides (Scheme 8). The dipeptides or amides may be produced (either in vitro or in vivo) by the use of a peptide synthetase enzyme, such as ACV synthetase (which may be modified by mutagenesis or other techniques to optimise dipeptide production) or by chemical synthesis. The process may include the following modifications:

N287F/A/G/V/L/W/M/C/N/Q/P/S/T/E/D/K/H/Y
R87F/A/G/V/L/I/W/M/C/N/Q/P/S/T/E/D/K/H/Y
Y189F/A/G/V/L/I/W/M/C/N/Q/P/S/T/E/D/K/H/R
S183F/A/G/V/L/I/W/M/C/N/Q/P/S/T/E/D/K/H/R/Y
Y91F/A/G/V/L/I/W/M/C/N/Q/P/S/T/E/D/K/H/R
F285A/G/V/L/I/W/M/C/N/Q/R/S/T/E/D/R/K/H/Y
Q330F/A/G/V/L/I/W/M/C/N/P/S/T/E/D/R/K/H/Y
T331F/A/G/V/L/I/W/M/C/N/P/S/E/D/R/K/H/Q/Y
V185F/A/G/L/I/W/M/C/N/P/S/T/E/D/R/K/H/Q/Y
L106F/A/G/V/I/W/M/C/N/P/S/T/E/D/R/K/H/Q/Y
C104F/A/G/V/L/I/W/M/N/P/S/T/E/D/R/K/H/Q/Y
V217F/A/G/L/I/W/M/C/N/P/S/T/E/D/R/K/H/Q/Y
L324F/A/G/V/I/W/M/C/N/P/S/T/E/D/R/K/H/Q/Y
L317F/A/G/V/I/W/M/C/N/P/S/T/E/D/R/K/H/Q/Y
I325F/A/G/V/I/W/M/C/N/P/S/Y/E/D/R/K/H/Q/Y
L321F/A/G/V/I/W/M/C/N/P/S/T/E/D/R/K/H/Q/Y
S210F/A/G/V/I/L/T/W/M/C/N/P/E/D/R/K/H/Q/Y

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, products, modifiers, or inhibitors are not excluded.

e) IPNS is modified to produce 3-exomethylenecephams with hydrophobic or other unnatural side chains (Scheme 9) (or other intermediates for use in the preparation of cephalosporin antibiotics, e.g. Cephachlor. The process will involve modification of both the side chain binding interactions of IPNS substrates and of the valine binding interactions and may involve the use of ACV as a substrate or the use of other unnatural substrates. The process may include the following modifications, which may be made in conjunction with each other:

N287F/A/G/V/L/I/W/M/C/N/Q/P/S/T/E/D/K/H/Y
R87F/A/G/V/L/I/W/M/C/N/Q/P/S/T/E/D/K/H/Y
Y189F/A/G/V/L/I/W/M/C/N/Q/P/S/T/E/D/K/H/R
S183F/A/G/V/L/I/W/M/C/M/Q/P/T/E/D/K/H/R/Y
Y91F/A/G/V/L/I/W/M/C/N/Q/P/S/T/E/D/K/H/R
F285A/G/V/L/I/W/M/C/N/Q/P/S/T/E/D/R/K/H/Y
Q330F/A/G/V/L/I/W/M/C/N/P/S/T/E/D/R/K/H/Y
T331F/A/G/V/L/I/W/M/C/N/P/S/E/D/R/K/H/Q/Y
V185F/A/G/L/I/W/M/C/N/P/S/T/E/D/R/K/H/Q/Y
L106F/A/G/L/I/W/M/C/N/P/S/T/E/D/R/K/H/Q/Y
C104F/A/G/V/L/I/W/M/N/P/S/T/E/D/R/K/H/Q/Y
V217F/A/G/L/I/W/M/C/N/P/S/T/E/D/R/K/H/Q/Y
L324F/A/G/V/I/W/M/C/N/P/S/T/E/D/R/K/H/Q/Y
L317F/A/G/V/I/W/M/C/N/P/S/T/E/D/R/K/H/Q/Y
I325F/A/G/V/L/W/M/C/N/P/S/T/E/D/R/K/H/Q/Y
L321F/A/G/V/I/W/M/C/N/P/S/T/E/D/R/K/H/Q/Y
V272F/A/G/I/L/W/M/C/N/P/S/T/E/D/R/K/H/Q/Y
L231F/A/G/V/I/W/M/C/N/P/S/T/E/D/R/K/H/Q/Y
L223F/A/G/V/I/W/M/C/N/P/S/T/E/D/R/K/H/Q/Y
P283F/A/G/V/I/L/W/M/C/N/S/T/E/D/R/K/H/Q/Y
T221F/A/G/V/I/L/W/M/C/N/P/S/E/D/R/K/H/Q/Y
F211A/G/V/I/L/W/M/C/N/P/S/T/E/D/R/K/H/Q/Y
I187F/A/G/L/W/M/C/N/P/S/T/E/D/R/K/H/Q/Y/V
V185F/A/G/I/L/W/M/C/N/P/S/T/E/D/R/K/H/Q/Y
Y189F/A/G/V/I/L/W/M/C/N/P/S/T/E/D/R/K/H/Q
R279F/A/G/V/I/L/W/M/C/N/P/S/T/E/D/K/H/Q/Y
S281F/A/G/V/I/L/W/M/C/N/P/T/E/D/R/K/H/Q/Y
N230F/A/G/V/I/L/W/M/C/P/S/T/E/D/R/K/H/Q/Y
Q225F/A/G/V/I/L/W/M/C/N/P/S/T/E/D/R/K/H/Y
N252F/A/G/V/I/L/W/M/C/P/S/T/E/D/R/K/H/Q/Y
S210F/A/G/V/I/L/T/W/M/C/N/P/E/D/R/K/H/Q/Y

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded. The use of a modified IPNS in conjunction with another modified or unmodified oxygenase enzyme (e.g. DAOCS. DACS, DAOC/DACS) is not excluded.

f) The structure of DAOCS is modified in its active interactions region to accept substrates (i.e. penicillins with hydrophobic side chains, (including, but not exclusively, penicillin G and penicillin V) to produce cephalosporins or other bicyclic β-lactams of commercial use with hydrophobic or other unnatural side chains (Scheme 10). The process may include the following modifications:

N287F/A/G/V/L/I/W/M/C/N/Q/P/S/T/E/D/K/H/Y
R87F/A/G/V/L/I/W/M/C/N/Q/P/S/T/E/D/K/H/Y
A98F/A/G/V/L/I/W/M/C/N/Q/P/S/T/E/D/K/H/Y
F189R/A/G/V/L/I/W/M/C/N/Q/P/S/F/E/D/K/H/Y
C183F/A/G/V/L/I/W/M/N/Q/R/T/E/D/K/H/R/Y/S
T91F/A/G/V/L/I/W/M/C/N/Q/P/S/E/D/K/H/R/Y
F285A/G/V/L/I/W/M/C/N/Q/P/S/T/E/D/R/K/H/Y
A330F/G/V/L/I/W/M/C/N/P/S/T/E/D/R/K/H/Y/Q
P185F/A/G/L/I/W/M/C/N/V/S/T/E/D/R/K/H/Q/Y
T104F/A/G/V/L/I/T/W/M/N/P/S/E/D/R/K/H/Q/Y
M217F/A/G/L/I/T/W/C/N/P/S/E/D/R/K/H/Q/Y/V
I324F/A/G/V/T/W/M/C/N/P/S/E/D/R/K/H/Q/Y/L
1317F/A/G/V/L/T/W/M/C/N/P/S/E/D/R/K/H/Q/Y
P325F/A/G/V/L/T/W/M/C/N/P/S/E/D/K/R/H/Q/Y/I
Y321F/A/G/V/I/T/W/M/C/N/P/S/E/D/K/H/Q/R/L
R210F/A/G/V/I/L/T/W/M/C/N/P/E/D/R/K/H/Q/Y/S
R190F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

g) The structure of DAOCS is modified in its active interactions region to accept natural or unnatural substrates (including, but not exclusively, penicillin N, isopenicillin N, adipoyl penicillin) to produce bicyclic β-lactams other than cephalosporins of commercial use. For example the region of DAOCS interacting with the thiazolidine ring of its natural substrate penicillin N may be modified such that the modified DAOCS produces 3-exomethylenecephams from penicillin N, penicillin G, or penicillin V, or other substrates for DAOCS (Scheme 11). The process may include the following modifications:

V272F/A/G/I/L/W/M/C/N/P/S/T/E/D/R/K/H/Q/Y
L231F/A/G/V/I/W/M/C/N/P/S/T/E/D/R/K/H/Q/Y
L223F/A/G/V/I/W/M/C/N/P/S/T/E/D/R/K/H/Q/Y
V283F/A/G/I/L/W/M/C/N/S/T/E/D/R/K/H/Q/Y/P
T221F/A/G/V/I/L/W/M/C/N/P/S/E/D/R/K/H/Q/Y
M211F/A/G/V/I/L/W/M/C/N/P/S/T/E/D/R/K/H/Q/Y
L187F/A/G/I/W/M/C/N/P/S/T/E/D/R/K/H/Q/Y/V
P185F/A/G/I/L/W/M/C/N/S/T/E/D/R/K/H/Q/Y/N
F189A/G/V/I/L/W/M/C/N/P/S/T/E/D/R/K/H/Q/Y
R279F/A/G/V/I/L/W/M/C/N/P/S/T/E/D/K/H/Q/Y
S281F/A/G V/I/L/W/M/C/N/P/T/E/D/R/K/H/Q/Y
N230F/A/G/V/I/L/W/M/C/P/S/T/E/D/R/K/H/Q/Y
Q225F/A/G/V/I/L/W/M/C/N/P/S/T/E/D/R/K/H/Y
F252F/A/G/V/I/L/W/M/C/P/S/T/E/D/R/K/H/Q/Y
R210F/A/G/V/I/L/T/W/M/C/N/P/E/D/R/K/H/Q/Y/S
R190F/A/G/V/I/L/T/W/M/C/N/P/E/D/R/K/H/Q/Y/S

It is recognised that these modifications may have to be made in conjunction with each other or with other modifications to DAOCS in order to produce a useful catalyst with the desired properties. Other modifications based on the use of the three dimensional structure of IPNS. DACS, DAOCS, DAOCS/DACS, other sequence related enzymes or complexes of these enzymes to their substrates, intermediates, modifiers, products or inhibitors are not excluded,

h) The side chain binding interactions of DAOCS are modified such that 6-aminopenicillins or other bicylic β-lactams may be produced in vitro or in vivo from 6-amino penicillins such as 6-aminopenicillanic acid (Scheme 12). The process may include the following modifications (other modifications based on the use of the three dimensional structures of IPNS or DAOCS or DAOCS/DACS are not excluded):

N287F/A/G/V/L/I/W/M/C/N/Q/P/S/T/E/D/K/H/Y
R87F/A/G/V/L/I/W/M/C/N/Q/P/S/T/E/D/K/H/Y
A88F/A/G/V/L/I/W/M/C/N/Q/P/S/T/E/D/K/H/Y
F189R/A/G/V/L/I/W/M/C/N/Q/P/S/T/E/D/K/H/Y
C183F/A/G/V/L/I/W/M/N/Q/P/T/E/D/K/H/R/Y/S
T91F/A/G/V/L/I/W/M/C/N/Q/P/S/E/D/K/H/R/Y
F285A/G/V/L/I/W/M/C/N/Q/P/S/T/E/D/R/K/H/Y
A330F/G/V/L/I/W/M/C/N/P/S/T/E/D/R/K/H/Y/Q
P185F/A/G/L/I/W/M/C/N/V/S/T/E/D/R/K/H/Q/Y
T104F/A/G/V/L/I/W/M/C/N/P/S/E/D/R/K/H/Q/Y
M217F/A/G/L/I/W/M/C/N/P/S/T/E/D/R/K/H/Q/Y
I324F/A/G/V/I/W/M/C/N/P/S/T/E/D/R/K/H/Q/Y
I317F/A/G/V/I/L/W/M/C/N/P/S/T/E/D/R/K/H/Q/Y
R325F/A/G/V/L/W/M/C/N/P/S/T/E/D/K/H/Q/Y/I
Y321F/A/G/V/I/L/W/M/C/N/P/S/T/E/D/R/K/H/Q
R210F/A/G/V/I/L/T/W/M/C/N/P/E/D/R/K/H/Q/Y/S
R190F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

i) The side chain binding interactions of DAOCS is modified such that cephams or cephalosporins without any substituent at the 7-position or other bicylic β-lactams, without any substituent at the 7-position, may be produced in vitro or in vivo from penicillins or cepham substrates (Scheme 13). The penicillanic acid may be produced whether in vitro or in vivo. The process may include the following modifications:

N287F/A/G/V/L/I/W/M/C/N/Q/P/S/T/E/D/K/H/Y
R87F/A/G/V/L/I/W/M/C/N/Q/P/S/T/E/D/K/H/Y
A88F/A/G/V/L/I/W/M/C/N/Q/P/S/T/E/D/K/H/Y
F189R/A/G/V/L/I/W/M/C/N/Q/P/S/T/E/D/K/H/Y
C183F/A/G/V/L/I/W/M/C/N/Q/P/T/E/D/K/H/R/Y/S
T91F/A/G/V/L/I/W/M/C/N/Q/P/S/E/D/K/H/R/Y
F285A/G/V/L/I/W/M/C/N/Q/P/S/T/E/D/R/K/H/Y
A330F/G/V/L/I/W/M/C/N/P/S/T/E/D/R/K/H/Y/Q
P185F/A/G/L/I/W/M/C/N/V/S/T/E/D/R/K/H/Q/Y
T104F/A/G/V/L/I/W/M/N/C/P/S/E/D/R/K/H/Q/Y
M217F/A/G/L/I/V/W/C/N/P/S/T/E/D/R/K/H/Q/Y
I324F/A/G/V/L/W/M/C/N/P/S/T/E/D/R/K/H/Q/Y
I317F/A/G/V/L/W/M/C/N/P/S/T/E/D/R/K/H/Q/Y
R325F/A/G/V/L/W/M/C/N/P/S/T/E/D/K/H/Q/Y/I
Y321F/A/G/V/I/L/W/M/C/N/P/S/T/E/D/R/K/H/Q
R210F/A/G/V/I/L/T/W/M/C/N/P/E/D/R/K/H/Q/Y/S
R190F/A/G/V/I/L/T/W/M/C/N/P/E/D/R/K/H/Q/T/S

It is recognised that the modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

j) DAOCS is modified to produce 3-exomethylenecephams with hydrophobic side chains (Scheme 14) (or other intermediates for use in the preparation of cephalosporin antibiotics, e.g. Cefachlor.) The process will involve modification of both the side chain binding interactions of DAOCS substrates and of the thiaxolidine binding interactions and may involve the use of penicillins with hydrophobic side chains (e.g. penicillin G or V) as substrates or the use of other unnatural substrates. The process may include the following modifications (other modifications based on the use of the three dimensional structures of IPNS or DAOCS or DAOCS/DACS are not excluded):

V272F/A/G/I/L/W/M/C/N/P/S/T/E/D/R/K/H/Q/Y
L231F/A/G/V/I/W/M/C/N/P/S/T/E/D/R/K/H/Q/Y
L223F/A/G/V/I/W/M/C/N/P/S/T/E/D/R/K/H/Q/Y
V283F/A/G/I/L/W/M/C/N/S/T/E/D/R/K/H/Q/Y/P
T221F/A/G/V/I/L/W/M/C/N/P/S/E/D/R/K/H/Q/Y
M211A/G/V/I/L/W/M/C/N/P/S/T/E/D/R/K/H/Q/Y
L187F/A/G/I/W/M/C/N/P/S/T/E/D/R/K/H/Q/Y/V
P185F/A/G/I/L/W/M/C/N/S/T/E/D/R/K/H/Q/Y/V
F189A/G/V/I/L/W/M/C/N/P/S/T/E/D/R/K/H/Q/Y
R279F/A/G/V/I/L/W/M/C/N/P/S/T/E/D/K/H/Q/Y
S281F/A/G/V/I/L/W/M/C/N/P/T/E/D/R/K/H/Q/Y
N230F/A/G/V/I/L/W/M/C/P/S/T/E/D/R/K/H/Q/Y
Q225F/A/G/V/I/L/W/M/C/N/P/S/T/E/D/R/K/H/Y
F252A/G/V/I/L/W/M/C/N/P/S/T/E/D/R/K/H/Q/Y
R287F/A/G/V/L/I/W/M/C/N/Q/P/S/T/E/D/K/H/Y
R87F/A/G/V/L/I/W/M/C/N/Q/P/S/T/E/D/K/H/Y
R88F/A/G/V/L/I/W/M/C/N/Q/P/S/T/E/D/K/H/Y
C183F/A/G/V/L/I/W/M/N/Q/P/T/E/D/K/H/R/Y/S
T91F/A/G/V/L/I/W/M/C/N/Q/P/S/E/D/K/H/R/Y
F285A/G/V/L/I/W/M/C/N/Q/P/S/T/E/D/R/K/H/Y
A330F/G/V/L/I/W/M/C/N/P/S/T/E/D/R/K/H/Y/Q
P185F/A/G/L/I/W/M/C/N/V/S/T/E/D/R/K/H/Q/Y
T104F/A/G/V/L/I/W/M/N/P/S/T/E/D/R/K/H/Q/Y
M217F/A/G/L/I/V/W/C/N/P/S/T/E/D/R/K/H/Q/Y
I324F/A/G/L/V/W/M/C/N/P/S/T/E/D/R/K/H/Q/Y
I317F/A/G/V/L/W/M/C/N/P/S/T/E/D/R/K/H/Q/Y
R325F/A/G/V/L/W/M/C/N/P/S/T/E/D/K/H/Q/Y/I
Y321F/A/G/V/I/W/M/C/N/P/S/T/E/D/R/K/H/Q/L
R210F/A/G/V/I/L/T/W/M/C/N/P/E/D/R/K/H/Q/Y/S
R190F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

k) The structure of DACS is modified in its active site region to accept substrates with hydrophobic side chains, including, but not exclusively, penicillin N, penicillin G and penicillin V) to produce cephalosporins or other bicyclic β-lactams of commercial use with hydrophobic or other unnatural side chains (Scheme 15). The process may include the following modifications:

N287F/A/G/V/L/I/W/M/C/N/Q/P/S/T/E/D/K/H/Y
R87F/A/G/V/L/I/W/M/C/N/Q/P/S/T/E/D/K/H/Y
A88F/A/G/V/L/I/W/M/C/N/Q/P/S/T/E/D/K/H/Y
F189F/A/G/V/L/I/W/M/C/N/Q/P/S/T/E/D/K/H/Y/R
C183F/A/G/V/L/I/W/M/N/Q/P/T/E/D/K/H/R/Y/S
S91F/A/G/V/L/I/W/M/C/N/Q/P/T/E/D/K/H/R/Y
F285A/G/V/L/I/W/M/C/N/Q/P/S/T/E/D/R/K/H/Y
A330F/G/V/L/I/W/M/C/N/P/S/T/E/D/R/K/H/Y/Q
P185F/A/G/L/I/W/M/C/N/V/S/T/E/D/R/K/H/Q/Y
T104F/A/G/V/L/I/W/M/N/P/S/E/D/R/K/H/Q/Y/C
L317F/A/G/V/I/W/M/C/N/P/S/T/E/D/R/K/H/Q/Y
R325F/A/G/V/L/W/M/C/N/P/S/T/E/D/K/H/Q/Y/I
Y321F/A/G/V/I/W/M/C/N/P/S/T/E/D/R/K/H/Q/L
R210F/A/G/V/I/L/T/W/M/C/N/P/E/D/R/K/H/Q/Y/S
R190F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

l) The structure of DACS is modified in its active site region to accept natural or unnatural substrates (including, but not exclusively, penicillin N, adipoyl penicillin) to produce bicyclic β-lactams other than cephalosporins of commercial use (Scheme 16). For example the region of DAOCS interacting with the thiazolidine ring of its natural substrate penicillin N may be modified such that the modified DAOCS produces 3-exomethylenecephams from penicillin N, penicillin G, or penicillin V, or other substrates for DAOCS. The process may include the following modifications

V272F/A/G/I/L/W/M/C/N/P/S/T/E/D/R/K/H/Q/Y
L231F/A/G/V/I/W/M/C/N/P/S/T/E/D/R/K/H/Q/Y
L223F/A/G/V/I/W/M/C/N/P/S/T/E/D/R/K/H/Q/Y
V283F/A/G/I/L/W/M/C/N/S/T/E/D/R/K/H/Q/Y/P
T221F/A/G/V/I/L/W/M/C/N/P/S/E/D/R/K/H/Q/Y
M211A/G/V/I/L/W/C/N/P/S/T/E/D/R/K/H/Q/Y/F
L187F/A/G/I/W/M/C/N/P/S/T/E/D/R/K/H/Q/Y/V
P185F/A/G/I/L/W/M/C/N/S/T/E/D/R/K/H/Q/Y/V
R279F/A/G/V/I/L/W/M/C/N/P/S/T/E/D/K/H/Q/Y
S281F/A/G/V/I/L/W/M/N/C/N/P/T/E/D/R/K/H/Q/Y
N230F/A/G/V/I/L/W/M/C/P/S/T/E/D/R/K/H/Q/Y
Q225F/A/G/V/I/L/W/M/N/C/N/P/S/T/E/D/R/K/H/Y
F252F/A/G/V/I/L/W/M/N/C/P/S/T/E/D/R/K/H/Q/Y
R210F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

m) The side chain binding interactions of DACS are modified such that 7-aminocephems or 7-aminocephams (including 3-exomethylencephams) or other bicylic β-lactams may be produced in vitro or in vivo from 6-amino penicillins (such as 6-aminopenicillanic acid) or cephams or cephems (Scheme 17). The process may include the following modifications:

N287F/A/G/V/L/I/W/M/C/N/Q/P/S/T/E/D/K/H/Y
R87F/A/G/V/L/I/W/M/C/N/Q/P/S/T/E/D/K/H/Y
A88F/A/G/V/L/I/W/M/C/N/Q/P/S/T/E/D/K/H/Y
F189R/A/G/V/L/I/W/M/C/N/Q/P/S/T/E/D/K/H/Y
C183F/A/G/V/L/I/W/M/N/Q/P/T/E/D/K/H/R/Y/S
S91F/A/G/V/L/I/W/M/C/N/Q/P/T/E/D/K/H/R/Y
F285A/G/V/L/I/W/M/C/N/Q/P/S/T/E/D/R/K/H/Y
A330F/G/V/L/I/W/M/C/N/P/S/T/E/D/R/K/H/Y/Q
P185F/A/G/L/I/W/M/C/N/V/S/T/E/D/R/K/H/Q/Y
T104F/A/G/V/L/I/W/M/N/P/S/E/D/R/K/H/Q/Y/C
L317F/A/G/V/I/W/M/C/N/P/S/T/E/D/R/K/H/Q/Y
R325F/A/G/V/L/W/M/C/N/P/S/T/E/D/K/H/Q/Y/I
Y321F/A/G/V/I/W/M/C/N/P/S/T/E/D/R/K/H/Q/L
R210F/A/G/V/I/L/T/W/M/C/N/P/E/D/R/K/H/Q/Y/S
R190F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

n) The side chain binding interactions of DACS are modified such that cephams or cephalosporins without any substituent at the 7-position or other bicylic β-lactams, without any substituent at the 7-position, may be produced in vitro or in vivo from penicillins or cepham substrates, such as penicillanic acid (Scheme 18). The penicillanic acid may be produced whether in vitro or in vivo. The process may include the following modifications:

N287F/A/G/V/L/I/W/M/C/N/Q/P/S/T/E/D/K/H/Y
R87F/A/G/V/L/I/W/M/C/N/Q/P/S/T/E/D/K/H/Y
A88F/A/G/V/L/I/W/M/C/N/Q/P/S/T/E/D/K/H/Y
F189R/A/G/V/L/I/W/M/C/N/Q/P/S/T/E/D/K/H/Y
C183F/A/G/V/L/I/W/M/N/Q/P/T/E/D/K/H/R/Y/S
S91F/A/G/V/L/I/W/M/C/N/Q/P/T/E/D/K/H/R/Y
F285A/G/V/L/I/W/M/C/N/Q/P/S/T/E/D/R/K/H/Y
A330F/G/V/L/I/W/M/C/N/P/S/T/E/D/R/K/H/Y/Q
P185F/A/G/L/I/W/M/C/N/V/S/T/E/D/R/K/H/Q/Y
T104F/A/G/V/L/I/W/M/N/P/S/E/D/R/K/H/Q/Y/C
L317F/A/G/V/I/W/M/C/N/P/S/T/E/D/R/K/H/Q/Y
R325F/A/G/V/L/W/M/C/N/P/S/T/E/D/K/H/Q/Y/I
Y321F/A/G/V/I/W/M/C/N/P/S/T/E/D/R/K/H/Q/L
R210F/A/G/V/I/L/T/W/M/C/N/P/E/D/R/K/H/Q/Y/S
R190F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

o) DACS is modified to produce 3-exomethylenecephams with hydrophobic side chains (or other intermediates for use in the preparation of cephalosporin antibiotics, e.g. Cephachlor.) (Scheme 19). The process will involve modification of both the side chain binding interactions of DACS substrates and of the thiaxolidine or cepham binding interactions and may involve the use of penicillins with hydrophobic side chains (e.g. penicillin G or V) as substrates or the use of other unnatural substrates. The process may include the following modifications:

V272F/A/G/I/L/W/M/C/N/P/S/T/E/D/R/K/H/Q/Y
L231F/A/G/V/I/W/M/C/N/P/S/T/E/D/R/K/H/Q/Y
L223F/A/G/V/I/W/M/C/N/P/S/T/E/D/R/K/H/Q/Y
V283F/A/G/I/L/W/M/C/N/S/T/E/D/R/K/H/Q/Y/P
T221F/A/G/V/I/L/W/M/C/N/P/S/E/D/R/K/H/Q/Y
M211A/G/V/I/L/W/C/N/P/S/T/E/D/R/K/H/Q/Y/F
L187F/A/G/I/L/W/M/C/N/S/T/E/D/R/K/H/Q/Y/V
P185F/A/G/I/L/W/M/C/N/S/T/E/D/R/K/H/Q/Y/V
R279F/A/G/V/I/L/W/M/C/N/P/S/T/E/D/K/H/Q/Y
S281F/A/G/V/I/L/W/M/N/C/N/P/T/E/D/R/K/H/Q/Y
N230F/A/G/V/I/L/W/M/C/P/S/T/E/D/R/K/H/Q/Y
Q225F/A/G/V/I/L/W/M/N/C/N/P/S/T/E/D/R/K/H/Y
F252F/A/G/V/I/L/W/M/N/C/P/S/T/E/D/R/K/H/Q/Y
R287F/A/G/V/L/I/W/M/C/N/Q/P/S/T/E/D/K/H/Y
R87F/A/G/V/L/I/W/M/C/N/Q/P/S/T/E/D/K/H/Y
R88F/A/G/V/L/I/W/M/C/N/Q/P/S/T/E/D/K/H/Y
F189R/A/G/V/L/I/W/M/C/N/Q/P/S/T/E/D/K/H/Y
C183F/A/G/V/L/I/W/M/N/Q/P/T/E/D/K/H/R/Y/S
S91F/A/G/V/L/I/W/M/C/N/Q/P/T/E/D/K/H/R/Y
F285A/G/V/L/I/W/M/C/N/Q/P/S/T/E/D/R/K/H/Y
A330F/G/V/L/I/W/M/C/N/P/S/T/E/D/R/K/H/Y/Q
P185F/A/G/L/I/W/M/C/N/V/S/T/E/D/R/K/H/Q/Y
T104F/A/G/V/L/I/W/M/N/P/S/E/D/R/K/H/Q/Y/C
L317F/A/G/V/I/W/M/C/N/P/S/T/E/D/R/K/H/Q/Y
R325F/A/G/V/L/W/M/C/N/P/S/T/E/D/K/H/Q/Y/I
Y321F/A/G/V/I/W/M/C/N/P/S/T/E/D/R/K/H/Q/L
R210F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

p) The structure of DAOCS/DACS is modified in its active site region to accept natural or unnatural substrates (including, but not exclusively, penicillin N, adipoyl penicillin) to produce bicyclic β-lactams other than cephalosporins of commercial use (Scheme 20). For example the region of DAOCS/DACS interacting with the thiazolidine ring of its natural substrate penicillin N (or the cepham ring of DAOC) may be modified such that the modified DAOCS/DACS produces 3-exomethylenecephams from penicillin N, penicillin G, or penicillin V, or other substrates for DAOCS/DACS. The process may include the following modifications:

V272F/A/G/I/L/W/M/C/N/P/S/T/E/D/R/K/H/Q/Y
L231F/A/G/V/I/W/M/C/N/P/S/T/E/D/R/K/H/Q/Y
L223F/A/G/V/I/W/M/C/N/P/S/T/E/D/R/K/H/Q/Y
V283F/A/G/I/L/W/M/C/N/S/T/E/D/R/K/H/Q/Y/P
T221F/A/G/V/I/L/W/M/C/N/P/S/E/D/R/K/H/Q/Y
M211A/G/V/I/L/T/W/C/N/P/S/E/D/R/K/H/Q/Y/F
L187F/A/G/I/T/W/M/C/N/P/S/E/D/R/K/H/Q/Y/V
P185F/A/G/I/L/T/W/M/C/N/S/E/D/R/K/H/Q/Y/V
L189A/G/V/I/L/T/W/M/C/N/P/S/E/D/R/K/H/Q/Y/F
R279F/A/G/V/I/L/T/W/M/C/N/P/S/E/D/K/H/Q/Y
S281F/A/G/V/I/L/T/W/M/C/N/P/E/D/R/K/H/Q/Y
N230F/A/G/V/I/L/T/W/M/C/P/S/E/D/R/K/H/Q/Y
Q225F/A/G/V/I/L/T/W/M/C/N/P/S/E/D/R/K/H/Y
F252F/A/G/V/I/L/T/W/M/C/P/S/E/D/R/K/H/Q/Y
R210F/A/G/V/I/L/T/W/M/C/N/P/E/D/R/K/H/Q/Y/S
R190F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

q) The side chain binding interactions of DAOCS/DACS are modified such that 7-aminocephems or 7-aminocephams (including 3-exomethylencephams) or other bicylic β-lactams may be produced in vitro or in vivo from 6-amino penicillins (e g. 6-aminopenicillanic acid) or cephams or cephems (Scheme 21). The process may include the following modifications:

N287F/A/G/V/L/I/W/M/C/N/Q/P/S/T/E/D/K/H/Y
R87F/A/G/V/L/I/W/M/C/N/Q/P/S/T/E/D/K/H/Y
A88F/A/G/V/L/I/W/M/C/N/Q/P/S/T/E/D/K/H/Y
L189F/A/G/V/L/I/W/M/C/N/Q/P/S/T/E/D/K/H/Y/R
C183F/A/G/V/L/I/W/M/N/Q/P/T/E/D/K/H/R/Y/S
S91F/A/G/V/L/I/W/M/C/N/Q/P/T/E/D/K/H/R/Y
F285A/G/V/L/I/W/M/C/N/Q/P/S/T/E/D/R/K/H/Y
A330F/G/V/L/I/W/M/C/N/P/S/T/E/D/R/K/H/Y/Q
P185F/A/G/L/I/W/M/C/N/V/S/T/E/D/R/K/H/Q/Y
T104F/A/G/V/L/I/W/M/C/N/P/S/E/D/R/K/H/Q/Y
T217F/A/G/L/I/V/W/M/C/N/P/S/E/D/R/K/H/Q/Y
M324F/A/G/V/I/L/W/C/N/P/S/T/E/D/R/K/H/Q/Y
L317F/A/G/V/I/W/M/C/N/P/S/T/E/D/R/K/H/Q/Y
R325F/A/G/V/L/W/M/C/N/P/S/T/E/D/K/H/Q/Y/I
Y321F/A/G/V/I/L/W/M/C/N/P/S/T/E/D/R/K/H/Q
R210F/A/G/V/I/L/T/W/M/C/N/P/E/D/R/K/H/Q/Y/S
R190F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

r) The side chain binding interactions of DAOCS/DACS are modified such that cephams or cephalosporins without any substituent at the 7-position or other bicylic β-lactams, without any substituent at the 7-position, may be produced in vitro or in vivo from penicillins or cepham substrates, such as penicillanic acid. The penicillanic acid may be produced whether in vitro or in vivo (Scheme 22). The process may include the following modifications:

N287F/A/G/V/L/I/W/M/C/N/Q/P/S/T/E/D/K/H/Y
R87F/A/G/V/L/I/W/M/C/N/Q/P/S/T/E/D/K/H/Y
A88F/A/G/V/L/I/W/M/C/N/Q/P/S/T/E/D/K/H/Y
L189F/A/G/V/L/I/W/M/C/N/Q/P/S/T/E/D/K/H/Y/R
C183F/A/G/V/L/I/W/M/N/Q/P/T/E/D/K/H/R/Y/S
S91F/A/G/V/L/I/W/M/C/N/Q/P/T/E/D/K/H/R/Y
F286A/G/V/L/I/W/M/C/N/Q/P/S/T/E/D/R/K/H/Y
A330F/G/V/L/I/W/M/C/N/P/S/T/E/D/R/K/H/Y/Q
P185F/A/G/L/I/W/M/C/N/V/S/T/E/D/R/K/H/Q/Y
T104F/A/G/V/L/I/W/M/C/N/P/S/E/D/R/K/H/Q/Y
T217F/A/G/L/I/V/W/M/C/N/P/S/E/D/R/K/H/Q/Y
M324F/A/G/V/I/L/W/C/N/P/S/T/E/D/R/K/H/Q/Y
L317F/A/G/V/I/W/M/C/N/P/S/T/E/D/R/K/H/Q/Y
R325F/A/G/V/L/W/M/C/N/P/S/T/E/D/K/H/Q/Y/I
Y321F/A/G/V/I/L/W/M/C/N/P/S/T/E/D/R/K/H/Q
R210F/A/G/V/I/L/T/W/M/C/N/P/E/D/R/K/H/Q/Y/S
R190F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

s) DAOCS/DACS is modified to produce 3-exomethylenecephams with hydrophobic side chains (or other intermediates for use in the preparation of cephalosporin antibiotics, e.g. Cephachlor) (Scheme 23). The process will involve modification or both the side chain binding interactions of DAOCS/DACS substrates and of the thiaxolidine or cepham binding interactions and may involve the use of penicillins with hydrophobic side chains (e.g. penicillin G or V) as substrates or the use of other unnatural substrates. The process may include the following modifications:

N287F/A/G/V/L/I/W/M/C/N/Q/P/S/T/E/D/K/H/Y
R87F/A/G/V/L/I/W/M/C/N/Q/P/S/T/E/D/K/H/Y
A88F/A/G/V/L/I/W/M/C/N/Q/P/S/T/E/D/K/H/Y
L189F/A/G/V/L/I/W/M/C/N/Q/P/S/T/E/D/K/H/Y/R
S91F/A/G/V/L/I/W/M/C/N/Q/P/T/E/D/K/H/R/Y
F285A/G/V/L/I/W/M/C/N/Q/P/S/T/E/D/R/K/H/Y
A330F/G/V/L/I/W/M/C/N/P/S/T/E/D/R/K/H/Y/Q
P185F/A/G/L/I/W/M/C/N/V/S/T/E/D/R/K/H/Q/Y
T104F/A/G/V/L/I/W/M/C/N/P/S/E/D/R/K/H/Q/Y
T217F/A/G/L/I/V/W/M/C/N/P/S/E/D/R/K/H/Q/Y
M324F/A/G/V/I/L/W/C/N/P/S/T/E/D/R/K/H/Q/Y
L317F/A/G/V/I/W/M/C/N/P/S/T/E/D/R/K/H/Q/Y
R325F/A/G/V/L/W/M/C/N/P/S/T/E/D/K/H/Q/Y/I
Y321F/A/G/V/I/L/W/M/C/N/P/S/T/E/D/R/K/H/Q
V272F/A/G/I/L/W/M/C/N/P/S/T/E/D/R/K/H/Q/Y
L231F/A/G/V/I/W/M/C/N/P/S/T/E/D/R/K/H/Q/Y
L223F/A/G/V/I/W/M/C/N/P/S/T/E/D/R/K/H/Q/Y
V283F/A/G/I/L/W/M/C/N/S/T/E/D/R/K/H/Q/Y/P
T221F/A/G/V/I/L/W/M/C/N/P/S/E/D/R/K/H/Q/Y
M211A/G/V/I/L/T/W/C/N/P/S/E/D/R/K/H/Q/Y/F
L187F/A/G/I/T/W/M/C/N/P/S/E/D/R/K/H/Q/Y/V
P185F/A/G/I/L/T/W/M/C/N/S/E/D/R/K/H/Q/Y/V
F189A/G/V/I/L/T/W/M/C/N/P/S/E/D/R/K/H/Q/Y
R279F/A/G/V/I/L/T/W/M/C/N/P/S/E/D/K/H/Q/Y
S281F/A/G/V/I/L/T/W/M/C/N/P/E/D/R/K/H/Q/Y
N230F/A/G/V/I/L/T/W/M/C/P/S/E/D/R/K/H/Q/Y
Q225F/A/G/V/I/L/T/W/M/C/N/P/S/E/D/R/K/H/Y
F252F/A/G/V/I/L/T/W/M/C/P/S/E/D/R/K/H/Q/Y
R210F/A/G/V/I/L/T/W/M/C/N/P/E/D/R/K/H/Q/Y/S
R190F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

t) The structure of DAOC/DACS is modified in its active site region to accept substrates (i.e. penicillins with hydrophobic side chains, (including, but not exclusively, penicillin N, penicillin G and penicillin V) to produce cephalosporins or other bicyclic β-lactams of commercial use with hydrophobic or other unnatural side chains (Scheme 24). The process may include the following modifications:

N287F/A/G/V/L/I/W/M/C/N/Q/P/S/T/E/D/K/H/Y
R87F/A/G/V/L/I/W/M/C/N/Q/P/S/T/E/D/K/H/Y
A88F/A/G/V/L/I/W/M/C/N/Q/P/S/T/E/D/K/H/Y
L189F/A/G/V/L/I/W/M/C/N/Q/P/S/T/E/D/K/H/Y/R
CF183F/A/G/V/L/I/W/M/N/Q/P/T/E/D/K/H/R/Y/S
S91F/A/G/V/L/I/W/M/C/N/Q/P/T/E/D/K/H/R/Y
F285A/G/V/L/I/W/M/C/N/Q/P/S/T/E/D/R/K/H/Y
A330F/G/V/L/I/W/M/C/N/P/S/T/E/D/R/K/H/Y/Q
P185F/A/G/L/I/W/M/C/N/V/S/T/E/D/R/K/H/Q/Y
T104F/A/G/V/L/I/W/M/C/N/P/S/E/D/R/K/H/Q/Y
T217F/A/G/L/I/V/W/M/C/N/P/S/E/D/R/K/H/Q/Y
M324F/A/G/V/I/L/W/C/N/P/S/T/E/D/R/K/H/Q/Y
L317F/A/G/V/I/W/M/C/N/P/S/T/E/D/R/K/H/Q/Y
R325F/A/G/V/L/W/M/C/N/P/S/T/E/D/K/H/Q/Y/I
Y321F/A/G/V/I/L/W/M/C/N/P/S/T/E/D/R/K/H/Q
R210F/A/G/V/I/L/T/W/M/C/N/P/E/D/R/K/H/Q/Y/S
R190F/A/G/V/I/L/T/W/M/C/N/P/E/D/R/K/H/Q/Y/S

It is recognised that these modifications may have to be made in conjunction with each other or with other modifications in order to produce a useful catalyst with the desired properties. Other modifications based on the use of the three dimensional structure of IPNS, DACS, DAOCS, DAOCS/DACS, other sequence related enzymes or complexes of these enzymes to their substrates, intermediates, modifiers, products or inhibitors are not excluded.

Use can also be made of the 3D structure of IPNS to determine or predict the structure of other related enzymes which are not active in the penicillin or cephalosporin biosynthesis pathway. The structural information so obtained can then be used to modify the other enzyme or for designing an inhibitor for the other enzymes. Such other enzymes include flavone synthase, prolyl hydroxylase, proline hydroxylase, lysyl hydroxylase, aspartyl hydroxylase, flvanone 3β-hydroxylase, gibberellin C-20 oxidase, gibberellin 3β-hydroxylase, para-hyroxyphenylpyruvate dioxygenase (HPPD), 1-aminocyclopropane-1-carboxylic acid (ACC) oxidase. Specific embodiments envisaged include:

The modification of the oxidases involved in gibberellin biosynthesis in order that modified enzymes may be introduced into plants in order to improve crop production.

The design of inhibitors of ACC oxidase to be used for the control of fruit ripening.

The design and use of inhibitors of prolyl hydroxylase for use in the treatment of arthritis and related diseases.

Modification of enzymes may conveniently be effected at the nucleic acid stage. Thus, the present invention envisages genes which code for the modified enzymes herein described. The nucleic acid sequence of such genes may be readily predicted. Mutations of existing wild-type genes may readily be effected e.g. by the use of commercially available mutagenesis kits.

The gene may be introduced into an expression vector by techniques which are well known. The expression vector may be used to transform a host microorganism, such as for example Penicillium chrysogenum or Acremonium chrysogenum, again by techniques which are well known. The micro-organism should be capable of expressing the gene under fermentation conditions, e.g. by having the gene under the transcriptional and translational regulation of fungal expression signals. Such micro-organisms containing the modified gene may be used to make bicyclic β-lactams of the penicillin or cephalosporin family, again by techniques which are well known.

The following experiments were performed to demonstrate the invention.

### EXAMPLE 1

A U.S.E mutagenesis kit (Phamacia) was used for all the mutagenesis reactions and a Pst I restriction site on the pET vector was selected. Selection of single and double mutants were successfully performed from colonies by restriction enzyme digestion. (Sambrook et al, Molecular Cloning, A Laboratory Manual, Cold Spring Harbour, USA, 1989). It was found that about 50% of colonies selected were mutants. Mutations of DAOCS (Table 1) were confirmed by sequencing according to the dideoxy method of Sanger. Mutants were designed after study of the IPNS-Mn²⁺ and the IPNS-Fe(II)-ACV structures. Polar residues with which the side chain D-α-aminoadipoyl (carboxylate and amino groups) might bind to were identified.

Almost all the mutants expressed well, except R88I, R88Q and R87Q/R287Q whereby the expression level was only about half of others. Generally the expression level of colonies was about 10˜20% of soluble protein at 27° C. Moreover, recombinant enzyme of P168V mutant was insoluble. These mutant enzymes were purified to ˜60-70% purity with Resouce-Q column (Phamacia). The activity of each mutant with respect to penicillin N and its side-chain analogues was analysed by bioassay. It was found that R87I, R87Q, R88I and R88Q could inhibit the growth of E. coli X580 cells using a hole-plate assay which contained penicillinase. The products of the reaction with penicillin G and wild type DAOCS also showed the same inhibition. Screening of the substrate conversion of penicillins mutants was also performed using a assay with radiolabelled α-ketoglutarate. The reaction conditions were the same as for bioassay except that [¹⁴C]-α-ketoglutarate was used. The specific activities of the various mutants are summarised in Table I.

The loss of activity when using penicillin N as a substrate after mutation of arginine 287 to isoleucine or glutamine in the active site of expandase implies an important interaction of this amino acid with the carboxyl group which located in the side chain of penicillin N. This is compatible with the structural predictions for DAOCS which were suggested based on IPNS structure. On the other hand, mutation at arginine 87 to isoleucine or glutamine enhanced the activity (when using penicillin N as a substrate), whereas mutants of arginine 88 caused partial loss of activity (when using penicillin N as a substrate). Double mutations at the sites totally eliminated activity.

The specific activities of the (mutant) modified DAOCS, when using penicillin N as a substrate, support the prediction that the 3-dimensional structure of DAOCS is closely related to that of IPNS. However, not all the kinetic results can be predicted by analysis of the predicted DAOC structure, e.g. the apparent increase in activity of the R87Q modification, when using penicillin N as a substrate. Other results in Table 1 further demonstrate the invention. For example the R87Q mutant converts penicillin G to phenylacetylcephalosporin G more efficiently than the unmodified enzyme. Other results demonstrate the introduction of new activities into the modified DAOCS enzymes. For example neither oxacillin nor piperacillin are substrates for the unmodified enzymes, but are substrates for the R87I/R287Q modified enzymes.

### EXAMPLE 2

IPNS-Fe-ACV Complex

Enzyme and Substrate Preparation

Recombinant A. nidulans IPNS was purified as the apo-enzyme as described previously (Roach et al, Protein Science, 1995, 4, 1007-1009) and stored at −80° C. in 75 μl aliquots (50 mg/ml in 20 mM Tris-HCl, pH 8.0). ACV (thiol form) was prepared as described previously and was further purified by HPLC [Hypersil octadecylsilane (C₁₈) column (250×10 mm), eluting with 10 mM NH₄CO₃, containing 4% (vol./vol.) MeOH; Rₜ=6.5 min at 4 ml/min], freeze dried and stored as 2 mg aliquots.

Crystallisation

Crystallisation trials were performed at 17° C. under anaerobic conditions (<0.2 ppm O₂) in a glove box (Belle Technology, Portesham, Dorset, UK) using the hanging drop vapour diffusion technique. All solutions except the protein were deoxygenated by repeated evacuation followed by argon flushing (repeated three times) prior to transfer to the anaerobic glove box. Solid reagents (ACV, ferrous sulphate and sodium dithionite), all solutions except protein solutions, washed cover-slips and greased Linbro plates were left for 16 h in the glove box to further deoxygenate. IPNS solutions were transferred to the glove box immediately prior to each crystallisation experiment and mixed by repeated gentle pipetting to assist deoxygenation. To further ensure that the crystallisation experiments were done anaerobically, a coloured redox indicator was added to each well. Thus, oxidised resazurin which shows a mauve to colourless change upon dithionate reduction, was added (0.001% mass/vol.) to the stock well solutions (separate solutions, without resazurin, were reserved for hanging drops) and sodium dithionite solution (100 mM) added dropwise until the solution in the well changed colour from mauve to colourless (Jacob, Methods in Microbiol., 1969, 2, 91-124). Upon exposure to oxygen (either by contamination or upon withdrawing the crystallisation tray from the glove box), the solution in the well changed from colourless (reduced) to pink (partially oxidised).

A stock solution containing ferrous sulphate (5 mM), ACV (80 mM) and IPNS (50 mg/ml. 1.35 mM) was then prepared and used in random screening experiments using 6 μl drops (1:1 precipitant:protein) (Jancerik and Kim, J. Appl. Crystallog., 1991, 24, 409). Three crystal forms were obtained using a precipitant solution containing 1.8M lithium sulphate and 100 mM Tris-HCl (pH 8.5). Crystals were not observed in analogous crystallisation experiments carried out in the absence of ACV. Crystallisation conditions were optimised by varying the protein and precipitant concentrations.

Plate crystals (Form I) typically appeared between 6 and 12 hours and reached a maximum size (typically 500×150×25 μm³) in 48 hours. Hexagonal columnar crystals (Form 11) typically appeared after 12-16 hours and grew to a maximum size (typically 1000×500×500 μm³) in 1 week. The needles (Form III), with a hexagonal cross-section, appeared after ca. 2 weeks and were more commonly observed when using less homogenous batches of protein. In analogous experiments carried out under aerobic conditions, no crystals were observed.

Form I crystals grew spontaneously in less than half of the drops after 12 hours. After this time, Form II crystals began to grow and predominated in those drops in which plates had not grown. By using serial dilutions of microseeds prepared from either Form I or Form II crystals, it was possible to bias the growth of crystals completely to either of these morphologies. There is a delicate balance between production of the different forms since some drops contained two or all three of the different crystal forms.

X-ray Analysis

For initial characterisation, crystals were mounted in quartz capillaries under an anaerobic atmosphere and the capillaries sealed with wax. Data were then collected (Table 4) at room temperature. Subsequently, the crystals were shown to be apparently stable to relatively short (<1 hour) exposure to oxygen and were withdrawn from the glove box. The crystals were then rapidly transferred to a cryoprotective mother liquor (100 mM Tris-HCl pH 8.5, 20% (vol./vol.) glycerol, saturated at room temperature with lithium sulphate) and frozen using a Cryostream (Oxford Cryosystems). Data were then collected at 100 K. Data were analysed using the programs DENZO and SCALEPACK (Otinowski, Data Collection and Processing, Daresbury Laboratory, Warrington, UK (Sawyer et al, Eds) PL/SCI/R34, pp 55-62).

TABLE 4
Crystal Statistics

| | Diffraction | | Unit Cell | Solvent | | |
|---|---|---|---|---|---|---|
| Crystal | Limit | Space | Dimensions | Content | Completeness | |
| Form | (nm) † | Group | (nm) | (%) | (%) | Rsym (%) |
| I | 0.11, 0.18 | P2₁2₁2₁ | 4.68, 7.15, 10.10 | 38.5 | 95.4 | 5.9 |
| II | 0.21, 0.23 | P3₁2₁ | 10.10, 10.10, | 69.5 | 94.0 | 7.2 |
| | | | 11.567 | | | |

† The first figure refers to the diffraction limit of the form I and form II crystals after respectively 30 and 10s exposures at BL19 of the European Synchrotron Radiation Facility (ESRF). The second figure refers to the diffraction limits after 30 min. exposures using a Rikagu rotating anode source operating at 60 kV and 70 mA equipped with a MAR Research imaging plate detector.
All other figures in the table refer to data collectedat the ESRF. The data for form I crystals was collected using a MAR Research imaging plate detector and the data for the form II crystals on a charged coupled device detector.

Hereafter:

Table 1 appears on page 41.

Table 2 appears on pages 42-78.

Table 3 appears on pages 79-119.

Reaction Schemes on pages 120-129.

TABLE 1
The Specific activity of various DAOCS mutants analysed by the turnover of α-[¹⁴C]-ketoglutarate.

| | | 6-Amino- | | | | | | | | | | |
|---|---|---|---|---|---|---|---|---|---|---|---|---|
| Specific | | peni- | | | | | | | | | | |
| activity | | cillanic | | | | | | | | | | |
| (nmol/ | Penicillin | acid | Adipyl- | Penicillin | Penicillin | Ampi- | Carbeni- | Ammoxi- | Methi- | Cloxa- | Oxa- | Pipera- |
| min/mg) | N | (6-APA) | 6-APA | G | V | cillin | cillin | cillin | cillin | cillin | cillin | cillin |
| Wild type | 6.4 ± | 1.0 ± 0.2 | 2.7 ± | 5.1 ± 0.4 | 5.3 ± 0.3 | 0 | 0 | 1.4 ± 0.6 | 0 | 1.6 ± 0.6 | 0 | 0 |
| | 0.5* | | 0.4 | | | | | | | | | |
| R287I | 0 | 1.7 ± 0.6 | 0 | 0 | 2.3 ± 0.2 | 0 | 0 | 1.1 ± 0.04 | 0.2 ± 0.1 | 2.7 ± 0.6 | 2.0 ± 0.09 | 1.8 ± 0.2 |
| R287Q | 0 | 0 | 0 | 0 | 0 | 0 | 0 | 0 | 0 | 0 | 0 | 0 |
| R87I | 6.4 ± | 1.1 ± 0.5 | 0 | 4.3 ± 0.1 | 1.5 ± 0.3 | 0 | 0 | 0 | 0 | 0.9 ± 0.04 | 0.8 ± 0.3 | 0 |
| | 0.05 | | | | | | | | | | | |
| R87Q | 13.4 ± 0.4 | 0 | 0 | 7.5 ± 0.4 | 3.4 ± 0.6 | 0 | 0 | 0 | 0 | 0 | 0 | 0 |
| R88I | 5.3 ± 0.8 | 0 | 0 | 2.5 ± 0.5 | 0 | 0 | 0 | 0 | 0 | 0 | 0 | 0 |
| R88Q | 2.9 ± 0.5 | 0 | 0 | 0.2 ± 0.02 | 0 | 0 | 0 | 0 | 0 | 0 | 0 | 0 |
| R87I/ | 0 | 0 | 0 | 0.3 ± 0.04 | 0 | 0 | 0 | 0 | 0 | 0 | 0 | 0 |
| R287I | | | | | | | | | | | | |
| R87Q/ | 0 | 0 | 0 | 0 | 0 | 0 | 0 | 0 | 0 | 0 | 0 | 0 |
| R287I | | | | | | | | | | | | |
| R87I/ | 0 | 3.3 ± 0.2 | 0 | 3.2 ± 0.1 | 0.7 ± 0.2 | 0 | 0 | 1.1 ± 0.3 | 0 | 4.5 ± 0.08 | 4.1 ± 0.4 | 2.5 ± 0.3 |
| R287Q | | | | | | | | | | | | |
| R87Q/ | 0 | 0 | 0 | 0 | 0 | 0 | 0 | 0 | 0 | 0 | 0 | 0 |
| R287Q | | | | | | | | | | | | |

*Experiments were done in duplicate and values for “the penicillin uncoupled decarboxylation of α-ketoglutarate” have been subtracted. The specific radioactivity of the α-ketoglutarate used was ca. 0.057 μCi/μmol. NB. “The penicillin uncoupled decarboxylation reaction” is the enzymatic turnover of α-ketoglutarate in the absence of pencillin substrate.

TABLE 2

| | | | | | | |
|---|---|---|---|---|---|---|
| CRYST1 | 59.200 | 127.000 | 139.600 | 90.00 | 90.00 | 90.00 |

| | | | | |
|---|---|---|---|---|
| SCALE1 | 0.016892 | 0.000000 | 0.000000 | 0.00000 |
| SCALE2 | 0.000000 | 0.007874 | 0.000000 | 0.00000 |
| SCALE3 | 0.000000 | 0.000000 | 0.007163 | 0.00000 |

| | | | | | | | | | | |
|---|---|---|---|---|---|---|---|---|---|---|
| ATOM | 1 | CB | VAL | A | 4 | 16.524 | 53.636 | −2.826 | 1.00 | 79.63 |
| ATOM | 2 | CG1 | VAL | A | 4 | 15.692 | 54.759 | −2.223 | 1.00 | 75.59 |
| ATOM | 3 | CG2 | VAL | A | 4 | 18.011 | 53.869 | −2.523 | 1.00 | 77.94 |
| ATOM | 4 | C | VAL | A | 4 | 14.636 | 52.001 | −2.797 | 1.00 | 78.66 |
| ATOM | 5 | O | VAL | A | 4 | 14.443 | 51.769 | −3.987 | 1.00 | 79.44 |
| ATOM | 6 | N | VAL | A | 4 | 16.880 | 51.117 | −2.818 | 1.00 | 82.21 |
| ATOM | 7 | CA | VAL | A | 4 | 16.049 | 52.254 | −2.290 | 1.00 | 80.32 |
| ATOM | 8 | N | SER | A | 5 | 13.655 | 52.015 | −1.916 | 1.00 | 76.44 |
| ATOM | 9 | CA | SER | A | 5 | 12.286 | 51.764 | −2.350 | 1.00 | 73.85 |
| ATOM | 10 | CB | SER | A | 5 | 11.583 | 50.804 | −1.380 | 1.00 | 74.99 |
| ATOM | 11 | OG | SER | A | 5 | 12.012 | 51.018 | −0.044 | 1.00 | 76.77 |
| ATOM | 12 | C | SER | A | 5 | 11.474 | 53.054 | −2.482 | 1.00 | 70.80 |
| ATOM | 13 | O | SER | A | 5 | 11.970 | 54.146 | −2.187 | 1.00 | 70.77 |
| ATOM | 14 | N | LYS | A | 6 | 10.250 | 52.914 | −2.970 | 1.00 | 67.33 |
| ATOM | 15 | CA | LYS | A | 6 | 9.320 | 54.025 | −3.124 | 1.00 | 64.23 |
| ATOM | 16 | CB | LYS | A | 6 | 8.403 | 53.799 | −4.319 | 1.00 | 65.78 |
| ATOM | 17 | CG | LYS | A | 6 | 8.751 | 54.568 | −5.557 | 1.00 | 69.46 |
| ATOM | 18 | CD | LYS | A | 6 | 7.579 | 54.445 | −6.510 | 1.00 | 76.71 |
| ATOM | 19 | CE | LYS | A | 6 | 7.768 | 55.261 | −7.784 | 1.00 | 81.89 |
| ATOM | 20 | NZ | LYS | A | 6 | 6.509 | 55.312 | −8.612 | 1.00 | 83.94 |
| ATOM | 21 | C | LYS | A | 6 | 8.457 | 54.095 | −1.868 | 1.00 | 61.05 |
| ATOM | 22 | O | LYS | A | 6 | 8.061 | 53.061 | −1.325 | 1.00 | 62.18 |
| ATOM | 23 | N | ALA | A | 7 | 8.166 | 55.304 | −1.410 | 1.00 | 55.41 |
| ATOM | 24 | CA | ALA | A | 7 | 7.346 | 55.487 | −0.231 | 1.00 | 49.08 |
| ATOM | 25 | CB | ALA | A | 7 | 7.632 | 56.853 | 0.393 | 1.00 | 44.79 |
| ATOM | 26 | C | ALA | A | 7 | 5.875 | 55.363 | −0.609 | 1.00 | 46.39 |
| ATOM | 27 | O | ALA | A | 7 | 5.469 | 55.706 | −1.721 | 1.00 | 44.68 |
| ATOM | 28 | N | ASN | A | 8 | 5.080 | 54.840 | 0.313 | 1.00 | 45.74 |
| ATOM | 29 | CA | ASN | A | 8 | 3.652 | 54.694 | 0.086 | 1.00 | 46.86 |
| ATOM | 30 | CB | ASN | A | 8 | 3.041 | 53.759 | 1.142 | 1.00 | 53.92 |
| ATOM | 31 | CG | ASN | A | 8 | 1.515 | 53.798 | 1.154 | 1.00 | 59.91 |
| ATOM | 32 | OD1 | ASN | A | 8 | 0.865 | 53.318 | 0.226 | 1.00 | 63.71 |
| ATOM | 33 | ND2 | ASN | A | 8 | 0.941 | 54.403 | 2.193 | 1.00 | 61.02 |
| ATOM | 34 | C | ASN | A | 8 | 3.009 | 56.078 | 0.175 | 1.00 | 45.51 |
| ATOM | 35 | O | ASN | A | 8 | 2.782 | 56.594 | 1.276 | 1.00 | 49.10 |
| ATOM | 36 | N | VAL | A | 9 | 2.802 | 56.712 | −0.977 | 1.00 | 42.13 |
| ATOM | 37 | CA | VAL | A | 9 | 2.167 | 58.028 | −1.026 | 1.00 | 36.18 |
| ATOM | 38 | CB | VAL | A | 9 | 3.066 | 59.093 | −1.733 | 1.00 | 31.81 |
| ATOM | 39 | CG1 | VAL | A | 9 | 2.425 | 60.459 | −1.650 | 1.00 | 28.09 |
| ATOM | 40 | CG2 | VAL | A | 9 | 4.438 | 59.149 | −1.100 | 1.00 | 26.59 |
| ATOM | 41 | C | VAL | A | 9 | 0.835 | 57.869 | −1.768 | 1.00 | 36.23 |
| ATOM | 42 | O | VAL | A | 9 | 0.785 | 57.827 | −3.000 | 1.00 | 39.84 |
| ATOM | 43 | N | PRO | A | 10 | −0.261 | 57.715 | −1.018 | 1.00 | 35.13 |
| ATOM | 44 | CD | PRO | A | 10 | −0.322 | 57.622 | 0.451 | 1.00 | 33.70 |
| ATOM | 45 | CA | PRO | A | 10 | −1.588 | 57.549 | −1.620 | 1.00 | 34.32 |
| ATOM | 46 | CB | PRO | A | 10 | −2.473 | 57.229 | −0.412 | 1.00 | 35.22 |
| ATOM | 47 | CG | PRO | A | 10 | −1.775 | 57.912 | 0.734 | 1.00 | 34.45 |
| ATOM | 48 | C | PRO | A | 10 | −2.094 | 58.759 | −2.390 | 1.00 | 33.02 |
| ATOM | 49 | O | PRO | A | 10 | −1.778 | 59.897 | −2.060 | 1.00 | 36.04 |
| ATOM | 50 | N | LYS | A | 11 | −2.870 | 58.503 | −3.434 | 1.00 | 35.45 |
| ATOM | 51 | CA | LYS | A | 11 | −3.435 | 59.576 | −4.236 | 1.00 | 36.88 |
| ATOM | 52 | CB | LYS | A | 11 | −3.361 | 59.233 | −5.724 | 1.00 | 31.82 |
| ATOM | 53 | CG | LYS | A | 11 | −1.958 | 58.944 | −6.203 | 1.00 | 38.51 |
| ATOM | 54 | CD | LYS | A | 11 | −1.858 | 58.929 | −7.722 | 1.00 | 44.67 |
| ATOM | 55 | CE | LYS | A | 11 | −0.482 | 58.455 | −8.166 | 1.00 | 47.02 |
| ATOM | 56 | NZ | LYS | A | 11 | 0.620 | 59.309 | −7.628 | 1.00 | 53.83 |
| ATOM | 57 | C | LYS | A | 11 | −4.882 | 59.740 | −3.798 | 1.00 | 39.16 |
| ATOM | 58 | O | LYS | A | 11 | −5.748 | 58.984 | −4.232 | 1.00 | 46.42 |
| ATOM | 59 | N | ILE | A | 12 | −5.133 | 60.704 | −2.917 | 1.00 | 36.94 |
| ATOM | 60 | CA | ILE | A | 12 | −6.474 | 60.959 | −2.394 | 1.00 | 32.90 |
| ATOM | 61 | CB | ILE | A | 12 | −6.407 | 61.510 | −0.965 | 1.00 | 25.23 |
| ATOM | 62 | CG2 | ILE | A | 12 | −7.803 | 61.826 | −0.436 | 1.00 | 19.49 |
| ATOM | 63 | CG1 | ILE | A | 12 | −5.682 | 60.505 | −0.077 | 1.00 | 26.81 |
| ATOM | 64 | CD1 | ILE | A | 12 | −5.414 | 60.995 | 1.314 | 1.00 | 29.30 |
| ATOM | 65 | C | ILE | A | 12 | −7.268 | 61.932 | −3.250 | 1.00 | 38.45 |
| ATOM | 66 | O | ILE | A | 12 | −6.729 | 62.919 | −3.749 | 1.00 | 41.39 |
| ATOM | 67 | N | ASP | A | 13 | −8.544 | 61.622 | −3.451 | 1.00 | 44.53 |
| ATOM | 68 | CA | ASP | A | 13 | −9.431 | 62.484 | −4.225 | 1.00 | 46.03 |
| ATOM | 69 | CB | ASP | A | 13 | −10.555 | 61.684 | −4.881 | 1.00 | 51.16 |
| ATOM | 70 | CG | ASP | A | 13 | −11.361 | 62.512 | −5.869 | 1.00 | 56.62 |
| ATOM | 71 | OD1 | ASP | A | 13 | −11.737 | 63.659 | −5.544 | 1.00 | 54.34 |
| ATOM | 72 | OD2 | ASP | A | 13 | −11.619 | 62.011 | −6.984 | 1.00 | 66.60 |
| ATOM | 73 | C | ASP | A | 13 | −10.000 | 63.472 | −3.227 | 1.00 | 44.39 |
| ATOM | 74 | O | ASP | A | 13 | −10.791 | 63.121 | −2.354 | 1.00 | 44.73 |
| ATOM | 75 | N | VAL | A | 14 | −9.605 | 64.719 | −3.391 | 1.00 | 44.33 |
| ATOM | 76 | CA | VAL | A | 14 | −10.003 | 65.785 | −2.498 | 1.00 | 38.69 |
| ATOM | 77 | CB | VAL | A | 14 | −8.796 | 66.720 | −2.338 | 1.00 | 34.66 |
| ATOM | 78 | CG1 | VAL | A | 14 | −9.190 | 68.101 | −1.875 | 1.00 | 37.77 |
| ATOM | 79 | CG2 | VAL | A | 14 | −7.833 | 66.077 | −1.365 | 1.00 | 33.42 |
| ATOM | 80 | C | VAL | A | 14 | −11.296 | 66.521 | −2.846 | 1.00 | 39.30 |
| ATOM | 81 | O | VAL | A | 14 | −11.808 | 67.298 | −2.036 | 1.00 | 39.59 |
| ATOM | 82 | N | SER | A | 15 | −11.891 | 66.202 | −3.990 | 1.00 | 38.33 |
| ATOM | 83 | CA | SER | A | 15 | −13.116 | 66.881 | −4.393 | 1.00 | 37.64 |
| ATOM | 84 | CB | SER | A | 15 | −13.686 | 66.298 | −5.689 | 1.00 | 39.61 |
| ATOM | 85 | OG | SER | A | 15 | −14.027 | 64.933 | −5.546 | 1.00 | 47.59 |
| ATOM | 86 | C | SER | A | 15 | −14.197 | 66.977 | −3.324 | 1.00 | 37.35 |
| ATOM | 87 | O | SER | A | 15 | −14.691 | 68.066 | −3.056 | 1.00 | 40.20 |
| ATOM | 88 | N | PRO | A | 16 | −14.532 | 65.866 | −2.647 | 1.00 | 38.58 |
| ATOM | 89 | CD | PRO | A | 16 | −13.957 | 64.511 | −2.714 | 1.00 | 40.22 |
| ATOM | 90 | CA | PRO | A | 16 | −15.574 | 65.918 | −1.613 | 1.00 | 37.63 |
| ATOM | 91 | CB | PRO | A | 16 | −15.410 | 64.583 | −0.901 | 1.00 | 33.25 |
| ATOM | 92 | CG | PRO | A | 16 | −14.999 | 63.689 | −1.991 | 1.00 | 36.58 |
| ATOM | 93 | C | PRO | A | 16 | −15.439 | 67.066 | −0.624 | 1.00 | 38.86 |
| ATOM | 94 | O | PRO | A | 16 | −16.442 | 67.614 | −0.184 | 1.00 | 40.71 |
| ATOM | 95 | N | LEU | A | 17 | −14.200 | 67.444 | −0.310 | 1.00 | 39.30 |
| ATOM | 96 | CA | LEU | A | 17 | −13.917 | 68.513 | 0.649 | 1.00 | 38.18 |
| ATOM | 97 | CB | LEU | A | 17 | −12.412 | 68.594 | 0.911 | 1.00 | 34.24 |
| ATOM | 98 | CG | LEU | A | 17 | −11.838 | 67.299 | 1.490 | 1.00 | 32.70 |
| ATOM | 99 | CD1 | LEU | A | 17 | −10.330 | 67.382 | 1.663 | 1.00 | 27.81 |
| ATOM | 100 | CD2 | LEU | A | 17 | −12.515 | 67.008 | 2.820 | 1.00 | 34.84 |
| ATOM | 101 | C | LEU | A | 17 | −14.472 | 69.881 | 0.260 | 1.00 | 41.67 |
| ATOM | 102 | O | LEU | A | 17 | −14.598 | 70.776 | 1.105 | 1.00 | 38.36 |
| ATOM | 103 | N | PHE | A | 18 | −14.774 | 70.043 | −1.025 | 1.00 | 48.11 |
| ATOM | 104 | CA | PHE | A | 18 | −15.339 | 71.287 | −1.551 | 1.00 | 52.21 |
| ATOM | 105 | CB | PHE | A | 18 | −14.857 | 71.551 | −2.993 | 1.00 | 51.38 |
| ATOM | 106 | CG | PHE | A | 18 | −13.365 | 71.738 | −3.132 | 1.00 | 49.52 |
| ATOM | 107 | CD1 | PHE | A | 18 | −12.552 | 70.679 | −3.513 | 1.00 | 50.16 |
| ATOM | 108 | CD2 | PHE | A | 18 | −12.781 | 72.983 | −2.932 | 1.00 | 47.59 |
| ATOM | 109 | CE1 | PHE | A | 18 | −11.183 | 70.857 | −3.695 | 1.00 | 50.47 |
| ATOM | 110 | CE2 | PHE | A | 18 | −11.413 | 73.166 | −3.114 | 1.00 | 44.37 |
| ATOM | 111 | CZ | PHE | A | 18 | −10.616 | 72.102 | −3.496 | 1.00 | 45.21 |
| ATOM | 112 | C | PHE | A | 18 | −16.871 | 71.202 | −1.550 | 1.00 | 53.42 |
| ATOM | 113 | O | PHE | A | 18 | −17.550 | 72.180 | −1.848 | 1.00 | 53.80 |
| ATOM | 114 | N | GLY | A | 19 | −17.407 | 70.020 | −1.259 | 1.00 | 56.82 |
| ATOM | 115 | CA | GLY | A | 19 | −18.847 | 69.842 | −1.247 | 1.00 | 60.49 |
| ATOM | 116 | C | GLY | A | 19 | −19.502 | 69.931 | 0.120 | 1.00 | 64.67 |
| ATOM | 117 | O | GLY | A | 19 | −18.927 | 70.470 | 1.071 | 1.00 | 64.98 |
| ATOM | 118 | N | ASP | A | 20 | −20.738 | 69.441 | 0.200 | 1.00 | 69.36 |
| ATOM | 119 | CA | ASP | A | 20 | −21.507 | 69.443 | 1.449 | 1.00 | 72.36 |
| ATOM | 120 | CB | ASP | A | 20 | −22.799 | 70.263 | 1.310 | 1.00 | 76.50 |
| ATOM | 121 | CG | ASP | A | 20 | −22.543 | 71.760 | 1.234 | 1.00 | 83.77 |
| ATOM | 122 | OD1 | ASP | A | 20 | −21.889 | 72.300 | 2.152 | 1.00 | 89.42 |
| ATOM | 123 | OD2 | ASP | A | 20 | −23.002 | 72.400 | 0.262 | 1.00 | 85.64 |
| ATOM | 124 | C | ASP | A | 20 | −21.861 | 68.035 | 1.918 | 1.00 | 70.68 |
| ATOM | 125 | O | ASP | A | 20 | −22.433 | 67.865 | 2.992 | 1.00 | 71.00 |
| ATOM | 126 | N | ASP | A | 21 | −21.533 | 67.030 | 1.111 | 1.00 | 68.45 |
| ATOM | 127 | CA | ASP | A | 21 | −21.830 | 65.653 | 1.473 | 1.00 | 66.91 |
| ATOM | 128 | CB | ASP | A | 21 | −21.643 | 64.720 | 0.267 | 1.00 | 69.88 |
| ATOM | 129 | CG | ASP | A | 21 | −22.015 | 63.268 | 0.574 | 1.00 | 73.83 |
| ATOM | 130 | OD1 | ASP | A | 21 | −22.477 | 62.978 | 1.702 | 1.00 | 76.66 |
| ATOM | 131 | OD2 | ASP | A | 21 | −21.845 | 62.409 | −0.322 | 1.00 | 76.24 |
| ATOM | 132 | C | ASP | A | 21 | −20.917 | 65.240 | 2.625 | 1.00 | 65.51 |
| ATOM | 133 | O | ASP | A | 21 | −19.785 | 64.800 | 2.419 | 1.00 | 67.20 |
| ATOM | 134 | N | GLN | A | 22 | −21.433 | 65.365 | 3.838 | 1.00 | 63.04 |
| ATOM | 135 | CA | GLN | A | 22 | −20.687 | 65.018 | 5.033 | 1.00 | 59.99 |
| ATOM | 136 | CB | GLN | A | 22 | −21.578 | 65.138 | 6.264 | 1.00 | 60.92 |
| ATOM | 137 | CG | GLN | A | 22 | −20.821 | 65.550 | 7.505 | 1.00 | 68.34 |
| ATOM | 138 | CD | GLN | A | 22 | −20.120 | 66.894 | 7.326 | 1.00 | 74.81 |
| ATOM | 139 | OE1 | GLN | A | 22 | −20.632 | 67.793 | 6.649 | 1.00 | 78.00 |
| ATOM | 140 | NE2 | GLN | A | 22 | −18.931 | 67.028 | 7.909 | 1.00 | 77.30 |
| ATOM | 141 | C | GLN | A | 22 | −20.104 | 63.623 | 4.971 | 1.00 | 58.00 |
| ATOM | 142 | O | GLN | A | 22 | −18.965 | 63.402 | 5.384 | 1.00 | 59.37 |
| ATOM | 143 | N | ALA | A | 23 | −20.877 | 62.688 | 4.428 | 1.00 | 55.74 |
| ATOM | 144 | CA | ALA | A | 23 | −20.448 | 61.294 | 4.323 | 1.00 | 57.85 |
| ATOM | 145 | CB | ALA | A | 23 | −21.550 | 60.444 | 3.688 | 1.00 | 57.01 |
| ATOM | 146 | C | ALA | A | 23 | −19.142 | 61.133 | 3.547 | 1.00 | 58.62 |
| ATOM | 147 | O | ALA | A | 23 | −18.180 | 60.534 | 4.040 | 1.00 | 59.15 |
| ATOM | 148 | N | ALA | A | 24 | −19.112 | 61.662 | 2.329 | 1.00 | 57.88 |
| ATOM | 149 | CA | ALA | A | 24 | −17.920 | 61.567 | 1.505 | 1.00 | 54.77 |
| ATOM | 150 | CB | ALA | A | 24 | −18.195 | 62.086 | 0.097 | 1.00 | 52.07 |
| ATOM | 151 | C | ALA | A | 24 | −16.772 | 62.334 | 2.176 | 1.00 | 52.15 |
| ATOM | 152 | O | ALA | A | 24 | −15.617 | 61.905 | 2.114 | 1.00 | 55.14 |
| ATOM | 153 | N | LYS | A | 25 | −17.097 | 63.443 | 2.835 | 1.00 | 46.23 |
| ATOM | 154 | CA | LYS | A | 25 | −16.087 | 64.230 | 3.516 | 1.00 | 42.19 |
| ATOM | 155 | CB | LYS | A | 25 | −16.690 | 65.505 | 4.112 | 1.00 | 38.59 |
| ATOM | 156 | CG | LYS | A | 25 | −16.655 | 66.663 | 3.149 | 1.00 | 33.56 |
| ATOM | 157 | CD | LYS | A | 25 | −17.022 | 67.980 | 3.806 | 1.00 | 35.11 |
| ATOM | 158 | CE | LYS | A | 25 | −18.525 | 68.172 | 3.890 | 1.00 | 37.82 |
| ATOM | 159 | NZ | LYS | A | 25 | −18.878 | 69.562 | 4.275 | 1.00 | 39.00 |
| ATOM | 160 | C | LYS | A | 25 | −15.406 | 63.406 | 4.593 | 1.00 | 43.50 |
| ATOM | 161 | O | LYS | A | 25 | −14.186 | 63.378 | 4.688 | 1.00 | 45.05 |
| ATOM | 162 | N | MET | A | 26 | −16.189 | 62.680 | 5.368 | 1.00 | 46.05 |
| ATOM | 163 | CA | MET | A | 26 | −15.599 | 61.872 | 6.424 | 1.00 | 51.52 |
| ATOM | 164 | CB | MET | A | 26 | −16.674 | 61.263 | 7.306 | 1.00 | 58.77 |
| ATOM | 165 | CG | MET | A | 26 | −17.065 | 62.138 | 8.503 | 1.00 | 68.62 |
| ATOM | 166 | SD | MET | A | 26 | −15.776 | 62.302 | 9.788 | 1.00 | 75.98 |
| ATOM | 167 | CE | MET | A | 26 | −15.385 | 60.571 | 10.146 | 1.00 | 72.86 |
| ATOM | 168 | C | MET | A | 26 | −14.740 | 60.785 | 5.816 | 1.00 | 49.91 |
| ATOM | 169 | O | MET | A | 26 | −13.709 | 60.391 | 6.395 | 1.00 | 49.46 |
| ATOM | 170 | N | ARG | A | 27 | −15.148 | 60.307 | 4.645 | 1.00 | 50.27 |
| ATOM | 171 | CA | ARG | A | 27 | −14.407 | 59.273 | 3.942 | 1.00 | 51.72 |
| ATOM | 172 | CB | ARG | A | 27 | −15.141 | 58.858 | 2.662 | 1.00 | 59.72 |
| ATOM | 173 | CG | ARG | A | 27 | −15.819 | 57.511 | 2.736 | 1.00 | 70.60 |
| ATOM | 174 | CD | ARG | A | 27 | −16.315 | 57.084 | 1.365 | 1.00 | 80.78 |
| ATOM | 175 | NE | ARG | A | 27 | −17.703 | 57.450 | 1.123 | 1.00 | 88.62 |
| ATOM | 176 | CZ | ARG | A | 27 | −18.115 | 58.133 | 0.056 | 1.00 | 93.71 |
| ATOM | 177 | NH1 | ARG | A | 27 | −17.243 | 58.547 | −0.867 | 1.00 | 96.83 |
| ATOM | 178 | NH2 | ARG | A | 27 | −19.414 | 58.338 | −0.135 | 1.00 | 97.09 |
| ATOM | 179 | C | ARG | A | 27 | −13.026 | 59.802 | 3.585 | 1.00 | 48.21 |
| ATOM | 180 | O | ARG | A | 27 | −12.030 | 59.115 | 3.794 | 1.00 | 49.76 |
| ATOM | 181 | N | VAL | A | 28 | −12.977 | 61.018 | 3.040 | 1.00 | 44.43 |
| ATOM | 182 | CA | VAL | A | 28 | −11.705 | 61.637 | 2.669 | 1.00 | 40.68 |
| ATOM | 183 | CB | VAL | A | 28 | −11.896 | 63.013 | 1.989 | 1.00 | 40.13 |
| ATOM | 184 | CG1 | VAL | A | 28 | −10.540 | 63.639 | 1.672 | 1.00 | 40.08 |
| ATOM | 185 | CG2 | VAL | A | 28 | −12.684 | 62.844 | 0.709 | 1.00 | 39.09 |
| ATOM | 186 | C | VAL | A | 28 | −10.868 | 61.798 | 3.922 | 1.00 | 41.23 |
| ATOM | 187 | O | VAL | A | 28 | −9.706 | 61.381 | 3.963 | 1.00 | 41.90 |
| ATOM | 188 | N | ALA | A | 29 | −11.510 | 62.271 | 4.981 | 1.00 | 40.14 |
| ATOM | 189 | CA | ALA | A | 29 | −10.854 | 62.492 | 6.255 | 1.00 | 39.42 |
| ATOM | 190 | CB | ALA | A | 29 | −11.873 | 62.936 | 7.274 | 1.00 | 40.42 |
| ATOM | 191 | C | ALA | A | 29 | −10.131 | 61.242 | 6.731 | 1.00 | 41.63 |
| ATOM | 192 | O | ALA | A | 29 | −8.963 | 61.307 | 7.119 | 1.00 | 42.66 |
| ATOM | 193 | N | GLN | A | 30 | −10.803 | 60.099 | 6.666 | 1.00 | 44.91 |
| ATOM | 194 | CA | GLN | A | 30 | −10.201 | 58.848 | 7.106 | 1.00 | 48.27 |
| ATOM | 195 | CB | GLN | A | 30 | −11.203 | 57.702 | 6.971 | 1.00 | 54.77 |
| ATOM | 196 | CG | GLN | A | 30 | −12.400 | 57.837 | 7.901 | 1.00 | 67.39 |
| ATOM | 197 | CD | GLN | A | 30 | −13.579 | 56.964 | 7.495 | 1.00 | 75.12 |
| ATOM | 198 | OE1 | GLN | A | 30 | −13.471 | 56.115 | 6.605 | 1.00 | 77.42 |
| ATOM | 199 | NE2 | GLN | A | 30 | −14.724 | 57.189 | 8.136 | 1.00 | 79.26 |
| ATOM | 200 | C | GLN | A | 30 | −8.930 | 58.544 | 6.328 | 1.00 | 47.85 |
| ATOM | 201 | O | GLN | A | 30 | −7.933 | 58.099 | 6.898 | 1.00 | 49.06 |
| ATOM | 202 | N | GLN | A | 31 | −8.972 | 58.807 | 5.025 | 1.00 | 45.74 |
| ATOM | 203 | CA | GLN | A | 31 | −7.820 | 58.573 | 4.164 | 1.00 | 42.76 |
| ATOM | 204 | CB | GLN | A | 31 | −8.188 | 58.781 | 2.701 | 1.00 | 40.15 |
| ATOM | 205 | CG | GLN | A | 31 | −9.129 | 57.723 | 2.175 | 1.00 | 43.01 |
| ATOM | 206 | CD | GLN | A | 31 | −9.468 | 57.922 | 0.715 | 1.00 | 48.15 |
| ATOM | 207 | OE1 | GLN | A | 31 | −8.717 | 57.518 | −0.166 | 1.00 | 52.25 |
| ATOM | 208 | NE2 | GLN | A | 31 | −10.609 | 58.541 | 0.449 | 1.00 | 55.93 |
| ATOM | 209 | C | GLN | A | 31 | −6.675 | 59.494 | 4.568 | 1.00 | 41.22 |
| ATOM | 210 | O | GLN | A | 31 | −5.547 | 59.042 | 4.765 | 1.00 | 43.52 |
| ATOM | 211 | N | ILE | A | 32 | −6.977 | 60.778 | 4.732 | 1.00 | 37.48 |
| ATOM | 212 | CA | ILE | A | 32 | −5.972 | 61.746 | 5.138 | 1.00 | 31.13 |
| ATOM | 213 | CB | ILE | A | 32 | −6.581 | 63.140 | 5.280 | 1.00 | 26.04 |
| ATOM | 214 | CG2 | ILE | A | 32 | −5.615 | 64.067 | 5.954 | 1.00 | 22.19 |
| ATOM | 215 | CG1 | ILE | A | 32 | −6.987 | 63.663 | 3.904 | 1.00 | 25.80 |
| ATOM | 216 | CD1 | ILE | A | 32 | −7.608 | 65.039 | 3.924 | 1.00 | 27.42 |
| ATOM | 217 | C | ILE | A | 32 | −5.343 | 61.308 | 6.458 | 1.00 | 34.56 |
| ATOM | 218 | O | ILE | A | 32 | −4.123 | 61.345 | 6.606 | 1.00 | 38.92 |
| ATOM | 219 | N | ASP | A | 33 | −6.169 | 60.833 | 7.387 | 1.00 | 36.94 |
| ATOM | 220 | CA | ASP | A | 33 | −5.684 | 60.375 | 8.689 | 1.00 | 38.24 |
| ATOM | 221 | CB | ASP | A | 33 | −6.850 | 59.928 | 9.588 | 1.00 | 44.05 |
| ATOM | 222 | CG | ASP | A | 33 | −6.380 | 59.328 | 10.930 | 1.00 | 47.52 |
| ATOM | 223 | OD1 | ASP | A | 33 | −5.824 | 60.066 | 11.773 | 1.00 | 44.85 |
| ATOM | 224 | OD2 | ASP | A | 33 | −6.583 | 58.111 | 11.149 | 1.00 | 46.89 |
| ATOM | 225 | C | ASP | A | 33 | −4.695 | 59.233 | 8.529 | 1.00 | 36.71 |
| ATOM | 226 | O | ASP | A | 33 | −3.654 | 59.209 | 9.182 | 1.00 | 39.40 |
| ATOM | 227 | N | ALA | A | 34 | −5.012 | 58.285 | 7.658 | 1.00 | 33.88 |
| ATOM | 228 | CA | ALA | A | 34 | −4.129 | 57.148 | 7.445 | 1.00 | 34.39 |
| ATOM | 229 | CB | ALA | A | 34 | −4.808 | 56.126 | 6.579 | 1.00 | 35.43 |
| ATOM | 230 | C | ALA | A | 34 | −2.783 | 57.560 | 6.841 | 1.00 | 38.07 |
| ATOM | 231 | O | ALA | A | 34 | −1.728 | 57.088 | 7.280 | 1.00 | 42.04 |
| ATOM | 232 | N | ALA | A | 35 | −2.817 | 58.454 | 5.851 | 1.00 | 37.24 |
| ATOM | 233 | CA | ALA | A | 35 | −1.596 | 58.933 | 5.197 | 1.00 | 34.56 |
| ATOM | 234 | CB | ALA | A | 35 | −1.941 | 59.864 | 4.054 | 1.00 | 31.14 |
| ATOM | 235 | C | ALA | A | 35 | −0.697 | 59.648 | 6.189 | 1.00 | 33.05 |
| ATOM | 236 | O | ALA | A | 35 | 0.485 | 59.347 | 6.295 | 1.00 | 36.62 |
| ATOM | 237 | N | SER | A | 36 | −1.276 | 60.589 | 6.923 | 1.00 | 32.76 |
| ATOM | 238 | CA | SER | A | 36 | −0.556 | 61.369 | 7.919 | 1.00 | 35.08 |
| ATOM | 239 | CB | SER | A | 36 | −1.503 | 62.396 | 8.544 | 1.00 | 30.17 |
| ATOM | 240 | OG | SER | A | 36 | −2.181 | 63.133 | 7.539 | 1.00 | 33.36 |
| ATOM | 241 | C | SER | A | 36 | 0.054 | 60.506 | 9.021 | 1.00 | 39.08 |
| ATOM | 242 | O | SER | A | 36 | 0.950 | 60.955 | 9.750 | 1.00 | 41.57 |
| ATOM | 243 | N | ARG | A | 37 | −0.456 | 59.288 | 9.172 | 1.00 | 38.47 |
| ATOM | 244 | CA | ARG | A | 37 | 0.053 | 58.394 | 10.191 | 1.00 | 38.11 |
| ATOM | 245 | CB | ARG | A | 37 | −1.095 | 57.702 | 10.908 | 1.00 | 40.18 |
| ATOM | 246 | CG | ARG | A | 37 | −1.866 | 58.642 | 11.805 | 1.00 | 47.64 |
| ATOM | 247 | CD | ARG | A | 37 | −3.157 | 58.021 | 12.262 | 1.00 | 55.80 |
| ATOM | 248 | NE | ARG | A | 37 | −2.923 | 56.776 | 12.976 | 1.00 | 66.31 |
| ATOM | 249 | CZ | ARG | A | 37 | −3.859 | 55.865 | 13.219 | 1.00 | 72.83 |
| ATOM | 250 | NH1 | ARG | A | 37 | −5.109 | 56.056 | 12.805 | 1.00 | 73.10 |
| ATOM | 251 | NH2 | ARG | A | 37 | −3.538 | 54.753 | 13.872 | 1.00 | 79.16 |
| ATOM | 252 | C | ARG | A | 37 | 1.035 | 57.393 | 9.627 | 1.00 | 38.74 |
| ATOM | 253 | O | ARG | A | 37 | 1.677 | 56.672 | 10.380 | 1.00 | 40.61 |
| ATOM | 254 | N | ASP | A | 38 | 1.151 | 57.349 | 8.305 | 1.00 | 40.43 |
| ATOM | 255 | CA | ASP | A | 38 | 2.086 | 56.440 | 7.658 | 1.00 | 41.47 |
| ATOM | 256 | CB | ASP | A | 38 | 1.435 | 55.783 | 6.437 | 1.00 | 49.45 |
| ATOM | 257 | CG | ASP | A | 38 | 2.199 | 54.556 | 5.951 | 1.00 | 58.59 |
| ATOM | 258 | OD1 | ASP | A | 38 | 2.821 | 53.855 | 6.784 | 1.00 | 62.36 |
| ATOM | 259 | OD2 | ASP | A | 38 | 2.162 | 54.281 | 4.732 | 1.00 | 62.50 |
| ATOM | 260 | C | ASP | A | 38 | 3.351 | 57.218 | 7.262 | 1.00 | 40.48 |
| ATOM | 261 | O | ASP | A | 38 | 4.213 | 57.461 | 8.105 | 1.00 | 36.69 |
| ATOM | 262 | N | THR | A | 39 | 3.449 | 57.618 | 5.991 | 1.00 | 41.01 |
| ATOM | 263 | CA | THR | A | 39 | 4.597 | 58.376 | 5.480 | 1.00 | 39.22 |
| ATOM | 264 | CB | THR | A | 39 | 4.675 | 58.298 | 3.948 | 1.00 | 39.18 |
| ATOM | 265 | OG1 | THR | A | 39 | 3.363 | 58.473 | 3.393 | 1.00 | 44.29 |
| ATOM | 266 | CG2 | THR | A | 39 | 5.221 | 56.968 | 3.519 | 1.00 | 43.28 |
| ATOM | 267 | C | THR | A | 39 | 4.497 | 59.847 | 5.850 | 1.00 | 37.79 |
| ATOM | 268 | O | THR | A | 39 | 5.505 | 60.538 | 5.973 | 1.00 | 41.79 |
| ATOM | 269 | N | GLY | A | 40 | 3.268 | 60.323 | 5.993 | 1.00 | 35.69 |
| ATOM | 270 | CA | GLY | A | 40 | 3.038 | 61.711 | 6.336 | 1.00 | 34.49 |
| ATOM | 271 | C | GLY | A | 40 | 2.842 | 62.524 | 5.078 | 1.00 | 32.99 |
| ATOM | 272 | O | GLY | A | 40 | 2.649 | 63.735 | 5.153 | 1.00 | 35.66 |
| ATOM | 273 | N | PHE | A | 41 | 2.867 | 61.842 | 3.932 | 1.00 | 32.44 |
| ATOM | 274 | CA | PHE | A | 41 | 2.713 | 62.465 | 2.617 | 1.00 | 30.35 |
| ATOM | 275 | CB | PHE | A | 41 | 3.986 | 62.260 | 1.780 | 1.00 | 23.42 |
| ATOM | 276 | CG | PHE | A | 41 | 5.094 | 63.225 | 2.094 | 1.00 | 19.36 |
| ATOM | 277 | CD1 | PHE | A | 41 | 6.079 | 62.899 | 3.013 | 1.00 | 19.44 |
| ATOM | 278 | CD2 | PHE | A | 41 | 5.161 | 64.455 | 1.454 | 1.00 | 20.61 |
| ATOM | 279 | CE1 | PHE | A | 41 | 7.120 | 63.790 | 3.292 | 1.00 | 21.76 |
| ATOM | 280 | CE2 | PHE | A | 41 | 6.192 | 65.350 | 1.723 | 1.00 | 19.48 |
| ATOM | 281 | CZ | PHE | A | 41 | 7.173 | 65.018 | 2.642 | 1.00 | 20.92 |
| ATOM | 282 | C | PHE | A | 41 | 1.558 | 61.855 | 1.840 | 1.00 | 30.54 |
| ATOM | 283 | O | PHE | A | 41 | 1.269 | 60.671 | 1.988 | 1.00 | 33.65 |
| ATOM | 284 | N | PHE | A | 42 | 0.900 | 62.662 | 1.016 | 1.00 | 29.43 |
| ATOM | 285 | CA | PHE | A | 42 | −0.179 | 62.172 | 0.171 | 1.00 | 28.45 |
| ATOM | 286 | CB | PHE | A | 42 | −1.473 | 61.872 | 0.953 | 1.00 | 28.60 |
| ATOM | 287 | CG | PHE | A | 42 | −2.292 | 63.083 | 1.332 | 1.00 | 26.83 |
| ATOM | 288 | CD1 | PHE | A | 42 | −3.186 | 63.655 | 0.431 | 1.00 | 26.89 |
| ATOM | 289 | CD2 | PHE | A | 42 | −2.218 | 63.612 | 2.613 | 1.00 | 28.60 |
| ATOM | 290 | CE1 | PHE | A | 42 | −3.998 | 64.734 | 0.803 | 1.00 | 22.24 |
| ATOM | 291 | CE2 | PHE | A | 42 | −3.030 | 64.692 | 2.993 | 1.00 | 28.04 |
| ATOM | 292 | CZ | PHE | A | 42 | −3.919 | 65.249 | 2.081 | 1.00 | 22.14 |
| ATOM | 293 | C | PHE | A | 42 | −0.416 | 63.122 | −0.979 | 1.00 | 30.71 |
| ATOM | 294 | O | PHE | A | 42 | −0.046 | 64.297 | −0.908 | 1.00 | 32.62 |
| ATOM | 295 | N | TYR | A | 43 | −0.911 | 62.580 | −2.084 | 1.00 | 31.35 |
| ATOM | 296 | CA | TYR | A | 43 | −1.200 | 63.386 | −3.257 | 1.00 | 27.25 |
| ATOM | 297 | CB | TYR | A | 43 | −0.851 | 62.641 | −4.556 | 1.00 | 25.54 |
| ATOM | 298 | CG | TYR | A | 43 | 0.573 | 62.844 | −5.054 | 1.00 | 21.56 |
| ATOM | 299 | CD1 | TYR | A | 43 | 1.524 | 61.841 | −4.942 | 1.00 | 19.07 |
| ATOM | 300 | CE1 | TYR | A | 43 | 2.835 | 62.034 | −5.386 | 1.00 | 17.94 |
| ATOM | 301 | CD2 | TYR | A | 43 | 0.966 | 64.047 | −5.628 | 1.00 | 23.58 |
| ATOM | 302 | CE2 | TYR | A | 43 | 2.272 | 64.246 | −6.068 | 1.00 | 15.91 |
| ATOM | 303 | CZ | TYR | A | 43 | 3.198 | 63.241 | −5.946 | 1.00 | 16.09 |
| ATOM | 304 | OH | TYR | A | 43 | 4.498 | 63.456 | −6.375 | 1.00 | 15.37 |
| ATOM | 305 | C | TYR | A | 43 | −2.673 | 63.735 | −3.249 | 1.00 | 29.51 |
| ATOM | 306 | O | TYR | A | 43 | −3.528 | 62.861 | −3.163 | 1.00 | 26.39 |
| ATOM | 307 | N | ALA | A | 44 | −2.960 | 65.026 | −3.252 | 1.00 | 32.50 |
| ATOM | 308 | CA | ALA | A | 44 | −4.327 | 65.497 | −3.290 | 1.00 | 32.14 |
| ATOM | 309 | CB | ALA | A | 44 | −4.448 | 66.834 | −2.586 | 1.00 | 31.97 |
| ATOM | 310 | C | ALA | A | 44 | −4.580 | 65.658 | −4.769 | 1.00 | 31.97 |
| ATOM | 311 | O | ALA | A | 44 | −3.968 | 66.506 | −5.414 | 1.00 | 33.03 |
| ATOM | 312 | N | VAL | A | 45 | −5.416 | 64.790 | −5.317 | 1.00 | 33.76 |
| ATOM | 313 | CA | VAL | A | 45 | −5.741 | 64.824 | −6.729 | 1.00 | 35.03 |
| ATOM | 314 | CB | VAL | A | 45 | −5.548 | 63.440 | −7.353 | 1.00 | 36.59 |
| ATOM | 315 | CG1 | VAL | A | 45 | −6.543 | 62.454 | −6.770 | 1.00 | 40.19 |
| ATOM | 316 | CG2 | VAL | A | 45 | −5.656 | 63.525 | −8.861 | 1.00 | 47.08 |
| ATOM | 317 | C | VAL | A | 45 | −7.184 | 65.291 | −6.866 | 1.00 | 36.63 |
| ATOM | 318 | O | VAL | A | 45 | −7.960 | 65.177 | −5.917 | 1.00 | 42.25 |
| ATOM | 319 | N | ASN | A | 46 | −7.538 | 65.823 | −8.034 | 1.00 | 36.71 |
| ATOM | 320 | CA | ASN | A | 46 | −8.883 | 66.349 | −8.294 | 1.00 | 35.51 |
| ATOM | 321 | CB | ASN | A | 46 | −9.956 | 65.320 | −7.942 | 1.00 | 42.00 |
| ATOM | 322 | CG | ASN | A | 46 | −10.436 | 64.547 | −9.144 | 1.00 | 51.81 |
| ATOM | 323 | OD1 | ASN | A | 46 | −11.513 | 64.813 | −9.671 | 1.00 | 58.83 |
| ATOM | 324 | ND2 | ASN | A | 46 | −9.641 | 63.582 | −9.588 | 1.00 | 54.06 |
| ATOM | 325 | C | ASN | A | 46 | −9.121 | 67.618 | −7.494 | 1.00 | 32.25 |
| ATOM | 326 | O | ASN | A | 46 | −10.206 | 67.848 | −6.996 | 1.00 | 35.38 |
| ATOM | 327 | N | HIS | A | 47 | −8.093 | 68.452 | −7.409 | 1.00 | 34.05 |
| ATOM | 328 | CA | HIS | A | 47 | −8.106 | 69.718 | −6.659 | 1.00 | 34.39 |
| ATOM | 329 | CB | HIS | A | 47 | −6.674 | 70.088 | −6.306 | 1.00 | 29.40 |
| ATOM | 330 | CG | HIS | A | 47 | −5.716 | 69.842 | −7.425 | 1.00 | 26.18 |
| ATOM | 331 | CD2 | HIS | A | 47 | −4.906 | 68.789 | −7.686 | 1.00 | 27.13 |
| ATOM | 332 | ND1 | HIS | A | 47 | −5.573 | 70.709 | −8.486 | 1.00 | 29.56 |
| ATOM | 333 | CE1 | HIS | A | 47 | −4.717 | 70.199 | −9.355 | 1.00 | 29.92 |
| ATOM | 334 | NE2 | HIS | A | 47 | −4.299 | 69.034 | −8.894 | 1.00 | 29.91 |
| ATOM | 335 | C | HIS | A | 47 | −8.740 | 70.890 | −7.397 | 1.00 | 35.30 |
| ATOM | 336 | O | HIS | A | 47 | −8.889 | 71.972 | −6.825 | 1.00 | 36.82 |
| ATOM | 337 | N | GLY | A | 48 | −8.955 | 70.710 | −8.700 | 1.00 | 36.56 |
| ATOM | 338 | CA | GLY | A | 48 | −9.578 | 71.732 | −9.528 | 1.00 | 37.05 |
| ATOM | 339 | C | GLY | A | 48 | −8.759 | 72.959 | −9.879 | 1.00 | 40.00 |
| ATOM | 340 | O | GLY | A | 48 | −9.321 | 74.004 | −10.196 | 1.00 | 46.68 |
| ATOM | 341 | N | ILE | A | 49 | −7.440 | 72.819 | −9.913 | 1.00 | 37.89 |
| ATOM | 342 | CA | ILE | A | 49 | −6.568 | 73.948 | −10.220 | 1.00 | 36.05 |
| ATOM | 343 | CB | ILE | A | 49 | −5.546 | 74.191 | −9.082 | 1.00 | 31.81 |
| ATOM | 344 | CG2 | ILE | A | 49 | −4.522 | 75.255 | −9.488 | 1.00 | 28.58 |
| ATOM | 345 | CG1 | ILE | A | 49 | −6.287 | 74.596 | −7.807 | 1.00 | 29.79 |
| ATOM | 346 | CD1 | ILE | A | 49 | −5.425 | 74.629 | −6.574 | 1.00 | 32.52 |
| ATOM | 347 | C | ILE | A | 49 | −5.815 | 73.686 | −11.514 | 1.00 | 38.13 |
| ATOM | 348 | O | ILE | A | 49 | −5.297 | 72.581 | −11.707 | 1.00 | 38.18 |
| ATOM | 349 | N | ASN | A | 50 | −5.749 | 74.701 | −12.383 | 1.00 | 38.60 |
| ATOM | 350 | CA | ASN | A | 50 | −5.050 | 74.603 | −13.663 | 1.00 | 38.30 |
| ATOM | 351 | CB | ASN | A | 50 | −5.453 | 75.726 | −14.619 | 1.00 | 42.37 |
| ATOM | 352 | CG | ASN | A | 50 | −4.920 | 75.502 | −16.033 | 1.00 | 46.03 |
| ATOM | 353 | OD1 | ASN | A | 50 | −4.258 | 74.496 | −16.314 | 1.00 | 46.67 |
| ATOM | 354 | ND2 | ASN | A | 50 | −5.195 | 76.445 | −16.922 | 1.00 | 49.68 |
| ATOM | 355 | C | ASN | A | 50 | −3.544 | 74.614 | −13.439 | 1.00 | 38.19 |
| ATOM | 356 | O | ASN | A | 50 | −2.853 | 75.631 | −13.581 | 1.00 | 36.43 |
| ATOM | 357 | N | VAL | A | 51 | −3.064 | 73.436 | −13.086 | 1.00 | 39.42 |
| ATOM | 358 | CA | VAL | A | 51 | −1.676 | 73.165 | −12.786 | 1.00 | 38.94 |
| ATOM | 359 | CB | VAL | A | 51 | −1.582 | 71.734 | −12.182 | 1.00 | 36.89 |
| ATOM | 360 | CG1 | VAL | A | 51 | −0.757 | 70.802 | −13.040 | 1.00 | 39.25 |
| ATOM | 361 | CG2 | VAL | A | 51 | −1.103 | 71.797 | −10.756 | 1.00 | 37.13 |
| ATOM | 362 | C | VAL | A | 51 | −0.804 | 73.346 | −14.025 | 1.00 | 40.42 |
| ATOM | 363 | O | VAL | A | 51 | 0.370 | 73.688 | −13.923 | 1.00 | 40.47 |
| ATOM | 364 | N | GLN | A | 52 | −1.404 | 73.193 | −15.198 | 1.00 | 43.48 |
| ATOM | 365 | CA | GLN | A | 52 | −0.658 | 73.335 | −16.439 | 1.00 | 46.46 |
| ATOM | 366 | CB | GLN | A | 52 | −1.461 | 72.753 | −17.607 | 1.00 | 54.80 |
| ATOM | 367 | CG | GLN | A | 52 | −1.828 | 71.265 | −17.427 | 1.00 | 64.12 |
| ATOM | 368 | CD | GLN | A | 52 | −0.603 | 70.340 | −17.338 | 1.00 | 70.98 |
| ATOM | 369 | OE1 | GLN | A | 52 | 0.494 | 70.696 | −17.774 | 1.00 | 73.27 |
| ATOM | 370 | NE2 | GLN | A | 52 | −0.799 | 69.140 | −16.788 | 1.00 | 71.68 |
| ATOM | 371 | C | GLN | A | 52 | −0.250 | 74.787 | −16.694 | 1.00 | 42.90 |
| ATOM | 372 | O | GLN | A | 52 | 0.932 | 75.081 | −16.876 | 1.00 | 42.08 |
| ATOM | 373 | N | ARG | A | 53 | −1.212 | 75.704 | −16.649 | 1.00 | 40.10 |
| ATOM | 374 | CA | ARG | A | 53 | −0.902 | 77.119 | −16.860 | 1.00 | 40.21 |
| ATOM | 375 | CB | ARG | A | 53 | −2.161 | 77.981 | −16.766 | 1.00 | 39.62 |
| ATOM | 376 | CG | ARG | A | 53 | −1.896 | 79.468 | −16.979 | 1.00 | 46.56 |
| ATOM | 377 | CD | ARG | A | 53 | −3.084 | 80.302 | −16.558 | 1.00 | 55.52 |
| ATOM | 378 | NE | ARG | A | 53 | −3.456 | 80.002 | −15.180 | 1.00 | 69.72 |
| ATOM | 379 | CZ | ARG | A | 53 | −4.707 | 79.854 | −14.750 | 1.00 | 75.43 |
| ATOM | 380 | NH1 | ARG | A | 53 | −5.728 | 79.988 | −15.589 | 1.00 | 79.20 |
| ATOM | 381 | NH2 | ARG | A | 53 | −4.936 | 79.529 | −13.485 | 1.00 | 80.30 |
| ATOM | 382 | C | ARG | A | 53 | 0.112 | 77.592 | −15.818 | 1.00 | 40.89 |
| ATOM | 383 | O | ARG | A | 53 | 0.967 | 78.436 | −16.103 | 1.00 | 42.68 |
| ATOM | 384 | N | LEU | A | 54 | 0.015 | 77.025 | −14.617 | 1.00 | 40.83 |
| ATOM | 385 | CA | LEU | A | 54 | 0.906 | 77.354 | −13.513 | 1.00 | 36.14 |
| ATOM | 386 | CB | LEU | A | 54 | 0.481 | 76.583 | −12.263 | 1.00 | 36.52 |
| ATOM | 387 | CG | LEU | A | 54 | 1.431 | 76.620 | −11.068 | 1.00 | 35.31 |
| ATOM | 388 | CD1 | LEU | A | 54 | 1.581 | 78.057 | −10.586 | 1.00 | 33.45 |
| ATOM | 389 | CD2 | LEU | A | 54 | 0.904 | 75.710 | −9.969 | 1.00 | 36.19 |
| ATOM | 390 | C | LEU | A | 54 | 2.380 | 77.073 | −13.829 | 1.00 | 36.31 |
| ATOM | 391 | O | LEU | A | 54 | 3.231 | 77.935 | −13.618 | 1.00 | 37.16 |
| ATOM | 392 | N | SER | A | 55 | 2.695 | 75.883 | −14.335 | 1.00 | 34.98 |
| ATOM | 393 | CA | SER | A | 55 | 4.090 | 75.558 | −14.645 | 1.00 | 36.10 |
| ATOM | 394 | CB | SER | A | 55 | 4.261 | 74.066 | −14.929 | 1.00 | 32.09 |
| ATOM | 395 | OG | SER | A | 55 | 3.071 | 73.521 | −15.455 | 1.00 | 41.05 |
| ATOM | 396 | C | SER | A | 55 | 4.618 | 76.377 | −15.804 | 1.00 | 37.67 |
| ATOM | 397 | O | SER | A | 55 | 5.789 | 76.752 | −15.825 | 1.00 | 41.07 |
| ATOM | 398 | N | GLN | A | 56 | 3.740 | 76.688 | −16.744 | 1.00 | 37.35 |
| ATOM | 399 | CA | GLN | A | 56 | 4.105 | 77.460 | −17.919 | 1.00 | 38.51 |
| ATOM | 400 | CB | GLN | A | 56 | 2.940 | 77.439 | −18.902 | 1.00 | 46.09 |
| ATOM | 401 | CG | GLN | A | 56 | 3.138 | 78.188 | −20.191 | 1.00 | 58.62 |
| ATOM | 402 | CD | GLN | A | 56 | 1.811 | 78.422 | −20.902 | 1.00 | 71.60 |
| ATOM | 403 | OE1 | GLN | A | 56 | 1.007 | 77.494 | −21.071 | 1.00 | 75.28 |
| ATOM | 404 | NE2 | GLN | A | 56 | 1.560 | 79.672 | −21.296 | 1.00 | 74.89 |
| ATOM | 405 | C | GLN | A | 56 | 4.496 | 78.893 | −17.560 | 1.00 | 36.41 |
| ATOM | 406 | O | GLN | A | 56 | 5.611 | 79.312 | −17.848 | 1.00 | 35.97 |
| ATOM | 407 | N | LYS | A | 57 | 3.599 | 79.629 | −16.905 | 1.00 | 35.19 |
| ATOM | 408 | CA | LYS | A | 57 | 3.869 | 81.015 | −16.514 | 1.00 | 34.22 |
| ATOM | 409 | CB | LYS | A | 57 | 2.710 | 81.567 | −15.693 | 1.00 | 35.10 |
| ATOM | 410 | CG | LYS | A | 57 | 1.443 | 81.768 | −16.469 | 1.00 | 41.47 |
| ATOM | 411 | CD | LYS | A | 57 | 1.644 | 82.849 | −17.492 | 1.00 | 49.86 |
| ATOM | 412 | CE | LYS | A | 57 | 0.507 | 82.868 | −18.477 | 1.00 | 58.96 |
| ATOM | 413 | NZ | LYS | A | 57 | 0.740 | 83.914 | −19.507 | 1.00 | 67.82 |
| ATOM | 414 | C | LYS | A | 57 | 5.147 | 81.106 | −15.691 | 1.00 | 36.94 |
| ATOM | 415 | O | LYS | A | 57 | 5.963 | 82.014 | −15.875 | 1.00 | 38.60 |
| ATOM | 416 | N | THR | A | 58 | 5.277 | 80.172 | −14.753 | 1.00 | 37.65 |
| ATOM | 417 | CA | THR | A | 58 | 6.426 | 80.066 | −13.865 | 1.00 | 35.48 |
| ATOM | 418 | CB | THR | A | 58 | 6.215 | 78.911 | −12.846 | 1.00 | 36.09 |
| ATOM | 419 | OG1 | THR | A | 58 | 5.257 | 79.317 | −11.862 | 1.00 | 30.91 |
| ATOM | 420 | CG2 | THR | A | 58 | 7.503 | 78.549 | −12.142 | 1.00 | 43.44 |
| ATOM | 421 | C | THR | A | 58 | 7.696 | 79.833 | −14.665 | 1.00 | 35.79 |
| ATOM | 422 | O | THR | A | 58 | 8.686 | 80.531 | −14.463 | 1.00 | 37.07 |
| ATOM | 423 | N | LYS | A | 59 | 7.667 | 78.865 | −15.577 | 1.00 | 38.20 |
| ATCM | 424 | CA | LYS | A | 59 | 8.832 | 78.573 | −16.397 | 1.00 | 40.23 |
| ATOM | 425 | CB | LYS | A | 59 | 8.540 | 77.444 | −17.391 | 1.00 | 46.70 |
| ATOM | 426 | CG | LYS | A | 59 | 9.744 | 77.071 | −18.254 | 1.00 | 58.45 |
| ATOM | 427 | CD | LYS | A | 59 | 9.534 | 75.783 | −19.053 | 1.00 | 69.21 |
| ATOM | 428 | CE | LYS | A | 59 | 10.831 | 75.350 | −19.769 | 1.00 | 76.48 |
| ATOM | 429 | NZ | LYS | A | 59 | 10.728 | 74.041 | −20.510 | 1.00 | 75.66 |
| ATOM | 430 | C | LYS | A | 59 | 9.199 | 79.846 | −17.134 | 1.00 | 40.20 |
| ATOM | 431 | O | LYS | A | 59 | 10.364 | 80.239 | −17.167 | 1.00 | 41.89 |
| ATOM | 432 | N | GLU | A | 60 | 8.186 | 80.531 | −17.653 | 1.00 | 41.30 |
| ATOM | 433 | CA | GLU | A | 60 | 8.395 | 81.777 | −18.379 | 1.00 | 42.90 |
| ATOM | 434 | CB | GLU | A | 60 | 7.059 | 82.362 | −18.851 | 1.00 | 50.15 |
| ATOM | 435 | CG | GLU | A | 60 | 6.405 | 81.626 | −20.026 | 1.00 | 57.31 |
| ATOM | 436 | CD | GLU | A | 60 | 5.215 | 82.383 | −20.605 | 1.00 | 63.82 |
| ATOM | 437 | OE1 | GLU | A | 60 | 4.233 | 81.733 | −21.027 | 1.00 | 68.21 |
| ATOM | 438 | OE2 | GLU | A | 60 | 5.259 | 83.633 | −20.644 | 1.00 | 67.21 |
| ATOM | 439 | C | GLU | A | 60 | 9.115 | 82.773 | −17.484 | 1.00 | 39.96 |
| ATOM | 440 | O | GLU | A | 60 | 10.147 | 83.324 | −17.859 | 1.00 | 42.99 |
| ATOM | 441 | N | PHE | A | 61 | 8.604 | 82.949 | −16.274 | 1.00 | 35.82 |
| ATOM | 442 | CA | PHE | A | 61 | 9.208 | 83.874 | −15.325 | 1.00 | 34.12 |
| ATOM | 443 | CB | PHE | A | 61 | 8.466 | 83.810 | −13.978 | 1.00 | 30.54 |
| ATOM | 444 | CG | PHE | A | 61 | 9.039 | 84.718 | −12.918 | 1.00 | 25.45 |
| ATOM | 445 | CD1 | PHE | A | 61 | 8.905 | 86.093 | −13.013 | 1.00 | 20.40 |
| ATOM | 446 | CD2 | PHE | A | 61 | 9.730 | 84.193 | −11.835 | 1.00 | 23.25 |
| ATOM | 447 | CE1 | PHE | A | 61 | 9.449 | 86.928 | −12.053 | 1.00 | 19.57 |
| ATOM | 448 | CE2 | PHE | A | 61 | 10.277 | 85.026 | −10.875 | 1.00 | 22.69 |
| ATOM | 449 | CZ | PHE | A | 61 | 10.133 | 86.399 | −10.989 | 1.00 | 17.25 |
| ATOM | 450 | C | PHE | A | 61 | 10.710 | 83.620 | −15.115 | 1.00 | 32.38 |
| ATOM | 451 | O | PHE | A | 61 | 11.536 | 84.499 | −15.353 | 1.00 | 30.20 |
| ATOM | 452 | N | HIS | A | 62 | 11.064 | 82.407 | −14.714 | 1.00 | 34.34 |
| ATOM | 453 | CA | HIS | A | 62 | 12.458 | 82.076 | −14.436 | 1.00 | 37.14 |
| ATOM | 454 | CB | HIS | A | 62 | 12.556 | 80.693 | −13.779 | 1.00 | 32.21 |
| ATOM | 455 | CG | HIS | A | 62 | 12.181 | 80.696 | −12.331 | 1.00 | 31.98 |
| ATOM | 456 | CD2 | HIS | A | 62 | 11.234 | 80.007 | −11.652 | 1.00 | 29.94 |
| ATOM | 457 | ND1 | HIS | A | 62 | 12.792 | 81.519 | −11.410 | 1.00 | 28.87 |
| ATOM | 458 | CE1 | HIS | A | 62 | 12.234 | 81.344 | −10.228 | 1.00 | 28.92 |
| ATOM | 459 | NE2 | HIS | A | 62 | 11.286 | 80.432 | −10.347 | 1.00 | 29.72 |
| ATOM | 460 | C | HIS | A | 62 | 13.437 | 82.193 | −15.599 | 1.00 | 41.35 |
| ATOM | 461 | O | HIS | A | 62 | 14.604 | 82.546 | −15.405 | 1.00 | 40.97 |
| ATOM | 462 | N | MET | A | 63 | 12.968 | 81.941 | −16.809 | 1.00 | 43.02 |
| ATOM | 463 | CA | MET | A | 63 | 13.867 | 82.018 | −17.941 | 1.00 | 45.13 |
| ATOM | 464 | CB | MET | A | 63 | 13.396 | 81.102 | −19.070 | 1.00 | 51.35 |
| ATOM | 465 | CG | MET | A | 63 | 13.180 | 79.653 | −18.631 | 1.00 | 63.00 |
| ATOM | 466 | SD | MET | A | 63 | 14.560 | 78.941 | −17.665 | 1.00 | 73.59 |
| ATOM | 467 | CE | MET | A | 63 | 15.229 | 77.786 | −18.858 | 1.00 | 73.56 |
| ATOM | 468 | C | MET | A | 63 | 14.052 | 83.438 | −18.445 | 1.00 | 44.26 |
| ATOM | 469 | O | MET | A | 63 | 15.126 | 83.781 | −18.927 | 1.00 | 50.29 |
| ATOM | 470 | N | SER | A | 64 | 13.041 | 84.283 | −18.287 | 1.00 | 39.05 |
| ATOM | 471 | CA | SER | A | 64 | 13.133 | 85.648 | −18.782 | 1.00 | 36.67 |
| ATOM | 472 | CB | SER | A | 64 | 11.798 | 86.062 | −19.386 | 1.00 | 37.55 |
| ATOM | 473 | OG | SER | A | 64 | 10.763 | 86.008 | −18.428 | 1.00 | 43.44 |
| ATOM | 474 | C | SER | A | 64 | 13.611 | 86.739 | −17.837 | 1.00 | 39.95 |
| ATOM | 475 | O | SER | A | 64 | 14.019 | 87.806 | −18.296 | 1.00 | 45.98 |
| ATOM | 476 | N | ILE | A | 65 | 13.486 | 86.531 | −16.530 | 1.00 | 41.66 |
| ATOM | 477 | CA | ILE | A | 65 | 13.914 | 87.547 | −15.568 | 1.00 | 37.23 |
| ATOM | 478 | CB | ILE | A | 65 | 13.477 | 87.209 | −14.108 | 1.00 | 35.14 |
| ATOM | 479 | CG2 | ILE | A | 65 | 14.228 | 86.007 | −13.559 | 1.00 | 26.54 |
| ATOM | 480 | CG1 | ILE | A | 65 | 13.725 | 88.412 | −13.207 | 1.00 | 31.65 |
| ATOM | 481 | CD1 | ILE | A | 65 | 12.960 | 88.365 | −11.914 | 1.00 | 37.39 |
| ATOM | 482 | C | ILE | A | 65 | 15.420 | 87.732 | −15.672 | 1.00 | 38.75 |
| ATOM | 483 | O | ILE | A | 65 | 16.165 | 86.757 | −15.710 | 1.00 | 43.09 |
| ATOM | 484 | N | THR | A | 66 | 15.857 | 88.980 | −15.785 | 1.00 | 38.80 |
| ATOM | 485 | CA | THR | A | 66 | 17.278 | 89.281 | −15.924 | 1.00 | 39.81 |
| ATOM | 486 | CB | THR | A | 66 | 17.486 | 90.544 | −16.776 | 1.00 | 40.56 |
| ATOM | 487 | OG1 | THR | A | 66 | 16.886 | 91.663 | −16.113 | 1.00 | 47.40 |
| ATOM | 488 | CG2 | THR | A | 66 | 16.854 | 90.371 | −18.139 | 1.00 | 41.17 |
| ATOM | 489 | C | THR | A | 66 | 17.948 | 89.502 | −14.580 | 1.00 | 39.84 |
| ATOM | 490 | O | THR | A | 66 | 17.291 | 89.829 | −13.597 | 1.00 | 46.85 |
| ATOM | 491 | N | PRO | A | 67 | 19.279 | 89.365 | −14.524 | 1.00 | 40.14 |
| ATOM | 492 | CD | PRO | A | 67 | 20.152 | 88.850 | −15.590 | 1.00 | 39.50 |
| ATOM | 493 | CA | PRO | A | 67 | 20.037 | 89.557 | −13.281 | 1.00 | 39.40 |
| ATOM | 494 | CB | PRO | A | 67 | 21.482 | 89.273 | −13.709 | 1.00 | 39.30 |
| ATOM | 495 | CG | PRO | A | 67 | 21.459 | 89.446 | −15.212 | 1.00 | 40.61 |
| ATOM | 496 | C | PRO | A | 67 | 19.884 | 90.924 | −12.604 | 1.00 | 39.27 |
| ATOM | 497 | O | PRO | A | 67 | 19.934 | 91.012 | −11.378 | 1.00 | 42.45 |
| ATOM | 498 | N | GLU | A | 68 | 19.704 | 91.986 | −13.387 | 1.00 | 37.98 |
| ATOM | 499 | CA | GLU | A | 68 | 19.528 | 93.316 | −12.811 | 1.00 | 36.08 |
| ATOM | 500 | CB | GLU | A | 68 | 19.598 | 94.425 | −13.865 | 1.00 | 44.41 |
| ATOM | 501 | CG | GLU | A | 68 | 20.830 | 94.408 | −14.745 | 1.00 | 56.01 |
| ATOM | 502 | CD | GLU | A | 68 | 20.701 | 93.426 | −15.897 | 1.00 | 62.86 |
| ATOM | 503 | OE1 | GLU | A | 68 | 19.776 | 93.599 | −16.727 | 1.00 | 65.53 |
| ATOM | 504 | OE2 | GLU | A | 68 | 21.519 | 92.480 | −15.972 | 1.00 | 66.08 |
| ATOM | 505 | C | GLU | A | 68 | 18.166 | 93.349 | −12.157 | 1.00 | 32.92 |
| ATOM | 506 | O | GLU | A | 68 | 17.968 | 94.037 | −11.158 | 1.00 | 37.34 |
| ATOM | 507 | N | GLU | A | 69 | 17.217 | 92.633 | −12.747 | 1.00 | 28.56 |
| ATOM | 508 | CA | GLU | A | 69 | 15.877 | 92.560 | −12.193 | 1.00 | 26.48 |
| ATOM | 509 | CB | GLU | A | 69 | 14.927 | 91.889 | −13.170 | 1.00 | 26.48 |
| ATOM | 510 | CG | GLU | A | 69 | 14.696 | 92.647 | −14.438 | 1.00 | 31.14 |
| ATOM | 511 | CD | GLU | A | 69 | 13.480 | 92.145 | −15.147 | 1.00 | 36.47 |
| ATOM | 512 | OE1 | GLU | A | 69 | 12.386 | 92.682 | −14.875 | 1.00 | 41.34 |
| ATOM | 513 | OE2 | GLU | A | 69 | 13.612 | 91.195 | −15.946 | 1.00 | 41.53 |
| ATOM | 514 | C | GLU | A | 69 | 15.925 | 91.749 | −10.900 | 1.00 | 26.61 |
| ATOM | 515 | O | GLU | A | 69 | 15.268 | 92.086 | −9.916 | 1.00 | 33.09 |
| ATOM | 516 | N | LYS | A | 70 | 16.703 | 90.672 | −10.902 | 1.00 | 23.31 |
| ATOM | 517 | CA | LYS | A | 70 | 16.830 | 89.844 | −9.719 | 1.00 | 18.07 |
| ATOM | 518 | CB | LYS | A | 70 | 17.730 | 88.655 | −10.000 | 1.00 | 16.10 |
| ATOM | 519 | CG | LYS | A | 70 | 17.125 | 87.693 | −10.978 | 1.00 | 16.50 |
| ATOM | 520 | CD | LYS | A | 70 | 18.081 | 86.602 | −11.323 | 1.00 | 19.40 |
| ATOM | 521 | CE | LYS | A | 70 | 17.421 | 85.611 | −12.243 | 1.00 | 23.23 |
| ATOM | 522 | NZ | LYS | A | 70 | 18.372 | 84.538 | −12.604 | 1.00 | 28.38 |
| ATOM | 523 | C | LYS | A | 70 | 17.397 | 90.685 | −8.596 | 1.00 | 22.66 |
| ATOM | 524 | O | LYS | A | 70 | 16.836 | 90.725 | −7.505 | 1.00 | 27.15 |
| ATOM | 525 | N | TRP | A | 71 | 18.461 | 91.424 | −8.891 | 1.00 | 21.96 |
| ATOM | 526 | CA | TRP | A | 71 | 19.101 | 92.274 | −7.897 | 1.00 | 22.52 |
| ATOM | 527 | CB | TRP | A | 71 | 20.321 | 92.982 | −8.494 | 1.00 | 19.42 |
| ATOM | 528 | CG | TRP | A | 71 | 21.037 | 93.865 | −7.506 | 1.00 | 20.03 |
| ATOM | 529 | CD2 | TRP | A | 71 | 21.800 | 93.441 | −6.366 | 1.00 | 17.51 |
| ATOM | 530 | CE2 | TRP | A | 71 | 22.293 | 94.604 | −5.736 | 1.00 | 15.91 |
| ATOM | 531 | CE3 | TRP | A | 71 | 22.103 | 92.194 | −5.809 | 1.00 | 19.70 |
| ATOM | 532 | CD1 | TRP | A | 71 | 21.104 | 95.230 | −7.524 | 1.00 | 18.18 |
| ATOM | 533 | NE1 | TRP | A | 71 | 21.859 | 95.680 | −6.466 | 1.00 | 21.55 |
| ATOM | 534 | CZ2 | TRP | A | 71 | 23.089 | 94.559 | −4.585 | 1.00 | 20.06 |
| ATOM | 535 | CZ3 | TRP | A | 71 | 22.897 | 92.147 | −4.662 | 1.00 | 20.49 |
| ATOM | 536 | CH2 | TRP | A | 71 | 23.373 | 93.324 | −4.061 | 1.00 | 20.53 |
| ATOM | 537 | C | TRP | A | 71 | 18.123 | 93.299 | −7.359 | 1.00 | 23.60 |
| ATOM | 538 | O | TRP | A | 71 | 18.089 | 93.584 | −6.155 | 1.00 | 24.12 |
| ATOM | 539 | N | ASP | A | 72 | 17.327 | 93.860 | −8.254 | 1.00 | 23.56 |
| ATOM | 540 | CA | ASP | A | 72 | 16.358 | 94.859 | 7.860 | 1.00 | 27.03 |
| ATOM | 541 | CB | ASP | A | 72 | 15.853 | 95.630 | −9.083 | 1.00 | 34.54 |
| ATOM | 542 | CG | ASP | A | 72 | 16.921 | 96.534 | −9.692 | 1.00 | 40.55 |
| ATOM | 543 | OD1 | ASP | A | 72 | 18.116 | 96.375 | −9.363 | 1.00 | 47.37 |
| ATOM | 544 | OD2 | ASP | A | 72 | 16.563 | 97.416 | −10.502 | 1.00 | 50.79 |
| ATOM | 545 | C | ASP | A | 72 | 15.208 | 94.309 | −7.032 | 1.00 | 26.53 |
| ATOM | 546 | O | ASP | A | 72 | 14.506 | 95.082 | −6.384 | 1.00 | 33.99 |
| ATOM | 547 | N | LEU | A | 73 | 15.055 | 92.989 | −6.999 | 1.00 | 23.44 |
| ATOM | 548 | CA | LEU | A | 73 | 13.998 | 92.353 | −6.224 | 1.00 | 19.61 |
| ATOM | 549 | CB | LEU | A | 73 | 13.219 | 91.372 | −7.091 | 1.00 | 20.50 |
| ATOM | 550 | CG | LEU | A | 73 | 12.333 | 91.920 | −8.201 | 1.00 | 20.73 |
| ATOM | 551 | CD1 | LEU | A | 73 | 11.692 | 90.760 | −8.945 | 1.00 | 10.89 |
| ATOM | 552 | CD2 | LEU | A | 73 | 11.280 | 92.830 | −7.601 | 1.00 | 14.36 |
| ATOM | 553 | C | LEU | A | 73 | 14.558 | 91.581 | −5.049 | 1.00 | 19.51 |
| ATOM | 554 | O | LEU | A | 73 | 13.811 | 91.098 | −4.212 | 1.00 | 22.35 |
| ATOM | 555 | N | ALA | A | 74 | 15.871 | 91.415 | −5.019 | 1.00 | 20.26 |
| ATOM | 556 | CA | ALA | A | 74 | 16.535 | 90.656 | −3.965 | 1.00 | 18.26 |
| ATOM | 557 | CB | ALA | A | 74 | 18.046 | 90.726 | −4.146 | 1.00 | 17.07 |
| ATOM | 558 | C | ALA | A | 74 | 16.163 | 91.106 | −2.569 | 1.00 | 19.57 |
| ATOM | 559 | O | ALA | A | 74 | 15.917 | 92.285 | −2.344 | 1.00 | 17.06 |
| ATOM | 560 | N | ILE | A | 75 | 16.115 | 90.157 | −1.637 | 1.00 | 21.97 |
| ATOM | 561 | CA | ILE | A | 75 | 15.811 | 90.457 | −0.239 | 1.00 | 18.37 |
| ATOM | 562 | CB | ILE | A | 75 | 15.337 | 89.202 | 0.514 | 1.00 | 15.88 |
| ATOM | 563 | CG2 | ILE | A | 75 | 14.056 | 88.700 | −0.073 | 1.00 | 15.90 |
| ATOM | 564 | CG1 | ILE | A | 75 | 16.380 | 88.096 | 0.422 | 1.00 | 17.42 |
| ATOM | 565 | CD1 | ILE | A | 75 | 16.178 | 86.992 | 1.424 | 1.00 | 20.14 |
| ATOM | 566 | C | ILE | A | 75 | 17.051 | 91.063 | 0.453 | 1.00 | 22.26 |
| ATOM | 567 | O | ILE | A | 75 | 18.155 | 91.028 | −0.093 | 1.00 | 24.15 |
| ATOM | 568 | N | ARG | A | 76 | 16.866 | 91.598 | 1.656 | 1.00 | 23.62 |
| ATOM | 569 | CA | ARG | A | 76 | 17.934 | 92.244 | 2.423 | 1.00 | 22.51 |
| ATOM | 570 | CB | ARG | A | 76 | 17.382 | 92.683 | 3.776 | 1.00 | 25.41 |
| ATOM | 571 | CG | ARG | A | 76 | 18.277 | 93.622 | 4.540 | 1.00 | 30.98 |
| ATOM | 572 | CD | ARG | A | 76 | 17.650 | 93.958 | 5.878 | 1.00 | 39.61 |
| ATOM | 573 | NE | ARG | A | 76 | 17.503 | 92.780 | 6.731 | 1.00 | 43.97 |
| ATOM | 574 | CZ | ARG | A | 76 | 16.615 | 92.675 | 7.719 | 1.00 | 46.12 |
| ATOM | 575 | NH1 | ARG | A | 76 | 15.786 | 93.678 | 7.995 | 1.00 | 44.59 |
| ATOM | 576 | NH2 | ARG | A | 76 | 16.533 | 91.550 | 8.417 | 1.00 | 44.99 |
| ATOM | 577 | C | ARG | A | 76 | 19.199 | 91.410 | 2.622 | 1.00 | 20.90 |
| ATOM | 578 | O | ARG | A | 76 | 20.303 | 91.939 | 2.667 | 1.00 | 21.19 |
| ATOM | 579 | N | ALA | A | 77 | 19.037 | 90.105 | 2.773 | 1.00 | 22.00 |
| ATOM | 580 | CA | ALA | A | 77 | 20.175 | 89.219 | 2.962 | 1.00 | 20.40 |
| ATOM | 581 | CB | ALA | A | 77 | 19.706 | 87.798 | 3.239 | 1.00 | 16.39 |
| ATOM | 582 | C | ALA | A | 77 | 21.127 | 89.245 | 1.770 | 1.00 | 24.32 |
| ATOM | 583 | O | ALA | A | 77 | 22.275 | 88.824 | 1.889 | 1.00 | 29.02 |
| ATOM | 584 | N | TYR | A | 78 | 20.643 | 89.704 | 0.618 | 1.00 | 25.15 |
| ATOM | 585 | CA | TYR | A | 78 | 21.471 | 89.795 | −0.587 | 1.00 | 19.05 |
| ATOM | 586 | CB | TYR | A | 78 | 20.810 | 89.103 | −1.764 | 1.00 | 16.11 |
| ATOM | 587 | CG | TYR | A | 78 | 20.748 | 87.618 | −1.613 | 1.00 | 16.70 |
| ATOM | 588 | CD1 | TYR | A | 78 | 19.611 | 87.000 | −1.114 | 1.00 | 15.71 |
| ATOM | 589 | CE1 | TYR | A | 78 | 19.542 | 85.626 | −1.006 | 1.00 | 14.79 |
| ATOM | 590 | CD2 | TYR | A | 78 | 21.821 | 86.823 | −1.987 | 1.00 | 17.98 |
| ATOM | 591 | CE2 | TYR | A | 78 | 21.762 | 85.458 | −1.886 | 1.00 | 15.81 |
| ATOM | 592 | CZ | TYR | A | 78 | 20.624 | 84.864 | −1.394 | 1.00 | 19.26 |
| ATOM | 593 | OH | TYR | A | 78 | 20.563 | 83.495 | −1.336 | 1.00 | 27.92 |
| ATOM | 594 | C | TYR | A | 78 | 21.735 | 91.233 | −0.952 | 1.00 | 18.90 |
| ATOM | 595 | O | TYR | A | 78 | 22.874 | 91.609 | −1.178 | 1.00 | 24.83 |
| ATOM | 596 | N | ASN | A | 79 | 20.672 | 92.025 | −1.046 | 1.00 | 20.12 |
| ATOM | 597 | CA | ASN | A | 79 | 20.778 | 93.442 | −1.396 | 1.00 | 23.74 |
| ATOM | 598 | CB | ASN | A | 79 | 19.767 | 93.794 | −2.491 | 1.00 | 21.09 |
| ATOM | 599 | CG | ASN | A | 79 | 19.985 | 95.173 | −3.071 | 1.00 | 20.38 |
| ATOM | 600 | OD1 | ASN | A | 79 | 20.558 | 96.049 | −2.437 | 1.00 | 24.59 |
| ATOM | 601 | ND2 | ASN | A | 79 | 19.511 | 95.378 | −4.283 | 1.00 | 17.81 |
| ATOM | 602 | C | ASN | A | 79 | 20.563 | 94.310 | −0.161 | 1.00 | 26.18 |
| ATOM | 603 | O | ASN | A | 79 | 19.442 | 94.645 | 0.206 | 1.00 | 28.37 |
| ATOM | 604 | N | LYS | A | 80 | 21.668 | 94.695 | 0.452 | 1.00 | 28.39 |
| ATOM | 605 | CA | LYS | A | 80 | 21.693 | 95.496 | 1.663 | 1.00 | 28.15 |
| ATOM | 606 | CB | LYS | A | 80 | 23.145 | 95.869 | 1.926 | 1.00 | 30.31 |
| ATOM | 607 | CG | LYS | A | 80 | 23.434 | 96.446 | 3.270 | 1.00 | 41.68 |
| ATOM | 608 | CD | LYS | A | 80 | 24.934 | 96.530 | 3.472 | 1.00 | 49.85 |
| ATOM | 609 | CE | LYS | A | 80 | 25.290 | 97.287 | 4.739 | 1.00 | 56.09 |
| ATOM | 610 | NZ | LYS | A | 80 | 26.764 | 97.261 | 4.971 | 1.00 | 59.86 |
| ATOM | 611 | C | LYS | A | 80 | 20.805 | 96.741 | 1.640 | 1.00 | 28.30 |
| ATOM | 612 | O | LYS | A | 80 | 20.388 | 97.234 | 2.679 | 1.00 | 31.85 |
| ATOM | 613 | N | GLU | A | 81 | 20.444 | 97.183 | 0.448 | 1.00 | 27.68 |
| ATOM | 614 | CA | GLU | A | 81 | 19.631 | 98.377 | 0.245 | 1.00 | 27.82 |
| ATOM | 615 | CB | GLU | A | 81 | 19.700 | 98.738 | −1.245 | 1.00 | 26.85 |
| ATOM | 616 | CG | GLU | A | 81 | 18.936 | 99.974 | −1.667 | 1.00 | 25.54 |
| ATOM | 617 | CD | GLU | A | 81 | 18.843 | 100.115 | −3.173 | 1.00 | 27.23 |
| ATOM | 618 | OE1 | GLU | A | 81 | 18.424 | 101.187 | −3.645 | 1.00 | 30.11 |
| ATOM | 619 | OE2 | GLU | A | 81 | 19.177 | 99.153 | −3.892 | 1.00 | 35.72 |
| ATOM | 620 | C | GLU | A | 81 | 18.163 | 98.261 | 0.685 | 1.00 | 28.54 |
| ATOM | 621 | O | GLU | A | 81 | 17.592 | 99.190 | 1.260 | 1.00 | 32.85 |
| ATOM | 622 | N | HIS | A | 82 | 17.544 | 97.125 | 0.400 | 1.00 | 28.51 |
| ATOM | 623 | CA | HIS | A | 82 | 16.145 | 96.919 | 0.736 | 1.00 | 26.34 |
| ATOM | 624 | CB | HIS | A | 82 | 15.547 | 95.839 | −0.148 | 1.00 | 23.16 |
| ATOM | 625 | CG | HIS | A | 82 | 15.992 | 95.898 | −1.569 | 1.00 | 20.08 |
| ATOM | 626 | CD2 | HIS | A | 82 | 16.006 | 96.906 | −2.467 | 1.00 | 14.88 |
| ATOM | 627 | ND1 | HIS | A | 82 | 16.428 | 94.781 | −2.241 | 1.00 | 21.04 |
| ATOM | 628 | CE1 | HIS | A | 82 | 16.684 | 95.094 | −3.493 | 1.00 | 20.58 |
| ATOM | 629 | NE2 | HIS | A | 82 | 16.433 | 96.382 | −3.661 | 1.00 | 16.40 |
| ATOM | 630 | C | HIS | A | 82 | 15.992 | 96.461 | 2.168 | 1.00 | 29.32 |
| ATOM | 631 | O | HIS | A | 82 | 15.653 | 95.302 | 2.415 | 1.00 | 29.56 |
| ATOM | 632 | N | GLN | A | 83 | 16.193 | 97.366 | 3.113 | 1.00 | 32.90 |
| ATOM | 633 | CA | GLN | A | 83 | 16.084 | 97.006 | 4.517 | 1.00 | 36.06 |
| ATOM | 634 | CB | GLN | A | 83 | 16.438 | 98.194 | 5.406 | 1.00 | 42.46 |
| ATOM | 635 | CG | GLN | A | 83 | 17.942 | 98.406 | 5.566 | 1.00 | 54.39 |
| ATOM | 636 | CD | GLN | A | 83 | 18.637 | 97.224 | 6.227 | 1.00 | 60.00 |
| ATOM | 637 | OE1 | GLN | A | 83 | 18.366 | 96.899 | 7.386 | 1.00 | 66.86 |
| ATOM | 638 | NE2 | GLN | A | 83 | 19.534 | 96.572 | 5.492 | 1.00 | 60.20 |
| ATOM | 639 | C | GLN | A | 83 | 14.746 | 96.418 | 4.932 | 1.00 | 34.12 |
| ATOM | 640 | O | GLN | A | 83 | 14.689 | 95.623 | 5.856 | 1.00 | 36.75 |
| ATOM | 641 | N | ASP | A | 84 | 13.684 | 96.755 | 4.215 | 1.00 | 35.26 |
| ATOM | 642 | CA | ASP | A | 84 | 12.353 | 96.260 | 4.546 | 1.00 | 35.02 |
| ATOM | 643 | CB | ASP | A | 84 | 11.293 | 97.298 | 4.158 | 1.00 | 47.91 |
| ATOM | 644 | CG | ASP | A | 84 | 11.437 | 98.611 | 4.925 | 1.00 | 61.72 |
| ATOM | 645 | OD1 | ASP | A | 84 | 11.115 | 99.673 | 4.344 | 1.00 | 68.07 |
| ATOM | 646 | OD2 | ASP | A | 84 | 11.863 | 98.587 | 6.104 | 1.00 | 69.22 |
| ATOM | 647 | C | ASP | A | 84 | 11.987 | 94.912 | 3.931 | 1.00 | 30.42 |
| ATOM | 648 | O | ASP | A | 84 | 10.890 | 94.402 | 4.158 | 1.00 | 31.96 |
| ATOM | 649 | N | GLN | A | 85 | 12.881 | 94.331 | 3.146 | 1.00 | 23.30 |
| ATOM | 650 | CA | GLN | A | 85 | 12.571 | 93.058 | 2.537 | 1.00 | 20.72 |
| ATOM | 651 | CB | GLN | A | 85 | 12.946 | 93.059 | 1.068 | 1.00 | 20.88 |
| ATOM | 652 | CG | GLN | A | 85 | 12.181 | 94.019 | 0.236 | 1.00 | 23.30 |
| ATOM | 653 | CD | GLN | A | 85 | 12.434 | 93.811 | −1.235 | 1.00 | 32.07 |
| ATOM | 654 | OE1 | GLN | A | 85 | 12.670 | 94.762 | −1.982 | 1.00 | 41.87 |
| ATOM | 655 | NE2 | GLN | A | 85 | 12.378 | 92.565 | −1.666 | 1.00 | 31.81 |
| ATOM | 656 | C | GLN | A | 85 | 13.286 | 91.930 | 3.242 | 1.00 | 22.01 |
| ATOM | 657 | O | GLN | A | 85 | 14.485 | 91.736 | 3.061 | 1.00 | 25.61 |
| ATOM | 658 | N | VAL | A | 86 | 12.551 | 91.171 | 4.038 | 1.00 | 21.04 |
| ATOM | 659 | CA | VAL | A | 86 | 13.151 | 90.063 | 4.758 | 1.00 | 20.72 |
| ATOM | 660 | CB | VAL | A | 86 | 12.835 | 90.127 | 6.271 | 1.00 | 23.91 |
| ATOM | 661 | CG1 | VAL | A | 86 | 13.522 | 88.996 | 6.993 | 1.00 | 24.41 |
| ATOM | 662 | CG2 | VAL | A | 86 | 13.272 | 91.445 | 6.856 | 1.00 | 24.17 |
| ATOM | 663 | C | VAL | A | 86 | 12.717 | 88.713 | 4.204 | 1.00 | 20.58 |
| ATOM | 664 | O | VAL | A | 86 | 13.554 | 87.851 | 3.990 | 1.00 | 29.31 |
| ATOM | 665 | N | ARG | A | 87 | 11.417 | 88.530 | 3.990 | 1.00 | 18.86 |
| ATOM | 666 | CA | ARG | A | 87 | 10.875 | 87.270 | 3.467 | 1.00 | 18.95 |
| ATOM | 667 | CB | ARG | A | 87 | 9.560 | 86.885 | 4.153 | 1.00 | 21.46 |
| ATOM | 668 | CG | ARG | A | 87 | 9.591 | 86.566 | 5.630 | 1.00 | 24.00 |
| ATOM | 669 | CD | ARG | A | 87 | 8.153 | 86.365 | 6.142 | 1.00 | 23.85 |
| ATOM | 670 | NE | ARG | A | 87 | 7.549 | 85.127 | 5.647 | 1.00 | 26.86 |
| ATOM | 671 | CZ | ARG | A | 87 | 6.252 | 84.833 | 5.742 | 1.00 | 24.59 |
| ATOM | 672 | NH1 | ARG | A | 87 | 5.412 | 85.691 | 6.300 | 1.00 | 22.47 |
| ATOM | 673 | NH2 | ARG | A | 87 | 5.803 | 83.654 | 5.333 | 1.00 | 22.94 |
| ATOM | 674 | C | ARG | A | 87 | 10.548 | 87.409 | 1.989 | 1.00 | 17.44 |
| ATOM | 675 | O | ARG | A | 87 | 10.947 | 86.591 | 1.181 | 1.00 | 20.34 |
| ATOM | 676 | N | ALA | A | 88 | 9.803 | 88.456 | 1.657 | 1.00 | 14.27 |
| ATOM | 677 | CA | ALA | A | 88 | 9.353 | 88.708 | 0.296 | 1.00 | 15.50 |
| ATOM | 678 | CB | ALA | A | 88 | 8.154 | 89.637 | 0.307 | 1.00 | 11.55 |
| ATOM | 679 | C | ALA | A | 88 | 10.413 | 89.247 | −0.630 | 1.00 | 18.22 |
| ATOM | 680 | O | ALA | A | 88 | 11.122 | 90.190 | −0.284 | 1.00 | 24.16 |
| ATOM | 681 | N | GLY | A | 89 | 10.461 | 88.688 | −1.837 | 1.00 | 18.31 |
| ATOM | 682 | CA | GLY | A | 89 | 11.437 | 89.103 | −2.825 | 1.00 | 18.37 |
| ATOM | 683 | C | GLY | A | 89 | 12.221 | 87.946 | −3.422 | 1.00 | 21.93 |
| ATOM | 684 | O | GLY | A | 89 | 11.853 | 86.773 | −3.262 | 1.00 | 23.18 |
| ATOM | 685 | N | TYR | A | 90 | 13.315 | 88.286 | −4.103 | 1.00 | 21.46 |
| ATOM | 686 | CA | TYR | A | 90 | 14.178 | 87.314 | −4.767 | 1.00 | 17.61 |
| ATOM | 687 | CB | TYR | A | 90 | 14.701 | 87.909 | −6.098 | 1.00 | 14.11 |
| ATOM | 688 | CG | TYR | A | 90 | 14.847 | 86.895 | −7.215 | 1.00 | 15.32 |
| ATOM | 689 | CD1 | TYR | A | 90 | 13.827 | 86.705 | −8.142 | 1.00 | 15.35 |
| ATOM | 690 | CE1 | TYR | A | 90 | 13.900 | 85.686 | −9.100 | 1.00 | 17.27 |
| ATOM | 691 | CD2 | TYR | A | 90 | 15.963 | 86.050 | −7.283 | 1.00 | 17.90 |
| ATOM | 692 | CE2 | TYR | A | 90 | 16.047 | 85.031 | −8.236 | 1.00 | 18.10 |
| ATOM | 693 | CZ | TYR | A | 90 | 15.010 | 84.855 | −9.136 | 1.00 | 18.33 |
| ATOM | 694 | OH | TYR | A | 90 | 15.056 | 83.829 | −10.056 | 1.00 | 29.46 |
| ATOM | 695 | C | TYR | A | 90 | 15.344 | 86.861 | −3.881 | 1.00 | 18.14 |
| ATOM | 696 | O | TYR | A | 90 | 15.877 | 87.636 | −3.089 | 1.00 | 19.67 |
| ATOM | 697 | N | TYR | A | 91 | 15.713 | 85.592 | −4.013 | 1.00 | 20.08 |
| ATOM | 698 | CA | TYR | A | 91 | 16.819 | 84.976 | −3.277 | 1.00 | 20.39 |
| ATOM | 699 | CB | TYR | A | 91 | 16.354 | 83.713 | −2.542 | 1.00 | 20.44 |
| ATOM | 700 | CG | TYR | A | 91 | 15.353 | 83.965 | −1.432 | 1.00 | 20.34 |
| ATOM | 701 | CD1 | TYR | A | 91 | 14.116 | 84.566 | −1.693 | 1.00 | 21.65 |
| ATOM | 702 | CE1 | TYR | A | 91 | 13.211 | 84.819 | −0.677 | 1.00 | 16.23 |
| ATOM | 703 | CD2 | TYR | A | 91 | 15.651 | 83.622 | −0.120 | 1.00 | 18.42 |
| ATOM | 704 | CE2 | TYR | A | 91 | 14.753 | 83.867 | 0.898 | 1.00 | 20.40 |
| ATOM | 705 | CZ | TYR | A | 91 | 13.536 | 84.468 | 0.615 | 1.00 | 22.22 |
| ATOM | 706 | OH | TYR | A | 91 | 12.651 | 84.705 | 1.639 | 1.00 | 21.37 |
| ATOM | 707 | C | TYR | A | 91 | 17.819 | 84.603 | −4.362 | 1.00 | 23.73 |
| ATOM | 708 | O | TYR | A | 91 | 17.583 | 83.687 | −5.154 | 1.00 | 24.20 |
| ATOM | 709 | N | LEU | A | 92 | 18.894 | 85.374 | −4.451 | 1.00 | 28.01 |
| ATOM | 710 | CA | LEU | A | 92 | 19.909 | 85.160 | −5.476 | 1.00 | 24.96 |
| ATOM | 711 | CB | LEU | A | 92 | 20.857 | 86.362 | −5.534 | 1.00 | 24.17 |
| ATOM | 712 | CG | LEU | A | 92 | 20.258 | 87.756 | −5.635 | 1.00 | 19.59 |
| ATOM | 713 | CD1 | LEU | A | 92 | 21.353 | 88.782 | −5.696 | 1.00 | 23.44 |
| ATOM | 714 | CD2 | LEU | A | 92 | 19.406 | 87.837 | −6.850 | 1.00 | 22.64 |
| ATOM | 715 | C | LEU | A | 92 | 20.749 | 83.910 | −5.283 | 1.00 | 25.80 |
| ATOM | 716 | O | LEU | A | 92 | 20.879 | 83.382 | −4.174 | 1.00 | 25.07 |
| ATOM | 717 | N | SER | A | 93 | 21.310 | 83.440 | −6.387 | 1.00 | 26.00 |
| ATOM | 718 | CA | SER | A | 93 | 22.210 | 82.307 | −6.358 | 1.00 | 27.05 |
| ATOM | 719 | CB | SER | A | 93 | 22.218 | 81.584 | −7.703 | 1.00 | 24.34 |
| ATOM | 720 | OG | SER | A | 93 | 22.434 | 82.479 | −8.776 | 1.00 | 26.84 |
| ATOM | 721 | C | SER | A | 93 | 23.569 | 82.942 | −6.087 | 1.00 | 27.46 |
| ATOM | 722 | O | SER | A | 93 | 23.776 | 84.125 | −6.348 | 1.00 | 26.92 |
| ATOM | 723 | N | ILE | A | 94 | 24.487 | 82.178 | −5.530 | 1.00 | 29.65 |
| ATOM | 724 | CA | ILE | A | 94 | 25.797 | 82.716 | −5.234 | 1.00 | 31.96 |
| ATOM | 725 | CB | ILE | A | 94 | 26.106 | 82.624 | −3.740 | 1.00 | 33.15 |
| ATOM | 726 | CG2 | ILE | A | 94 | 27.467 | 83.228 | −3.456 | 1.00 | 29.48 |
| ATOM | 727 | CG1 | ILE | A | 94 | 25.025 | 83.353 | −2.946 | 1.00 | 33.35 |
| ATOM | 728 | CD1 | ILE | A | 94 | 25.093 | 83.120 | −1.463 | 1.00 | 36.58 |
| ATOM | 729 | C | ILE | A | 94 | 26.753 | 81.863 | −6.027 | 1.00 | 34.38 |
| ATOM | 730 | O | ILE | A | 94 | 26.946 | 80.691 | −5.717 | 1.00 | 36.74 |
| ATOM | 731 | N | PRO | A | 95 | 27.339 | 82.423 | −7.080 | 1.00 | 38.50 |
| ATOM | 732 | CD | PRO | A | 95 | 27.368 | 83.872 | −7.350 | 1.00 | 40.61 |
| ATOM | 733 | CA | PRO | A | 95 | 28.283 | 81.724 | −7.947 | 1.00 | 40.90 |
| ATOM | 734 | CB | PRO | A | 95 | 28.917 | 82.854 | −8.745 | 1.00 | 45.71 |
| ATOM | 735 | CG | PRO | A | 95 | 28.765 | 84.049 | −7.829 | 1.00 | 45.59 |
| ATOM | 736 | C | PRO | A | 95 | 29.309 | 80.929 | −7.159 | 1.00 | 39.51 |
| ATOM | 737 | O | PRO | A | 95 | 30.004 | 81.477 | −6.310 | 1.00 | 42.47 |
| ATOM | 738 | N | GLY | A | 96 | 29.327 | 79.625 | −7.391 | 1.00 | 38.51 |
| ATOM | 739 | CA | GLY | A | 96 | 30.259 | 78.752 | −6.713 | 1.00 | 35.63 |
| ATOM | 740 | C | GLY | A | 96 | 29.740 | 78.161 | −5.424 | 1.00 | 37.97 |
| ATOM | 741 | O | GLY | A | 96 | 30.126 | 77.062 | −5.047 | 1.00 | 39.52 |
| ATOM | 742 | N | LYS | A | 97 | 28.812 | 78.849 | −4.777 | 1.00 | 35.33 |
| ATOM | 743 | CA | LYS | A | 97 | 28.318 | 78.365 | −3.504 | 1.00 | 34.40 |
| ATOM | 744 | CB | LYS | A | 97 | 28.555 | 79.419 | −2.435 | 1.00 | 42.78 |
| ATOM | 745 | CG | LYS | A | 97 | 29.982 | 79.900 | −2.352 | 1.00 | 54.46 |
| ATOM | 746 | CD | LYS | A | 97 | 30.041 | 81.101 | −1.428 | 1.00 | 65.72 |
| ATOM | 747 | CE | LYS | A | 97 | 31.450 | 81.651 | −1.284 | 1.00 | 73.04 |
| ATOM | 748 | NZ | LYS | A | 97 | 31.498 | 82.894 | −0.447 | 1.00 | 78.91 |
| ATOM | 749 | C | LYS | A | 97 | 26.857 | 77.959 | −3.450 | 1.00 | 32.83 |
| ATOM | 750 | O | LYS | A | 97 | 26.501 | 77.068 | −2.683 | 1.00 | 34.44 |
| ATOM | 751 | N | LYS | A | 98 | 26.008 | 78.603 | −4.243 | 1.00 | 26.62 |
| ATOM | 752 | CA | LYS | A | 98 | 24.574 | 78.319 | −4.215 | 1.00 | 22.39 |
| ATOM | 753 | CB | LYS | A | 98 | 23.876 | 79.351 | −3.319 | 1.00 | 23.74 |
| ATOM | 754 | CG | LYS | A | 98 | 22.362 | 79.269 | −3.276 | 1.00 | 22.19 |
| ATOM | 755 | CD | LYS | A | 98 | 21.766 | 80.300 | −2.335 | 1.00 | 22.81 |
| ATOM | 756 | CE | LYS | A | 98 | 20.251 | 80.160 | −2.241 | 1.00 | 22.30 |
| ATOM | 757 | NZ | LYS | A | 98 | 19.547 | 80.977 | −3.253 | 1.00 | 21.47 |
| ATOM | 758 | C | LYS | A | 98 | 23.990 | 78.346 | −5.614 | 1.00 | 21.39 |
| ATOM | 759 | O | LYS | A | 98 | 24.076 | 79.355 | −6.301 | 1.00 | 23.12 |
| ATOM | 760 | N | ALA | A | 99 | 23.427 | 77.225 | −6.044 | 1.00 | 21.69 |
| ATOM | 761 | CA | ALA | A | 99 | 22.819 | 77.132 | −7.369 | 1.00 | 23.49 |
| ATOM | 762 | CB | ALA | A | 99 | 23.126 | 75.756 | −7.983 | 1.00 | 21.77 |
| ATOM | 763 | C | ALA | A | 99 | 21.301 | 77.449 | −7.439 | 1.00 | 22.78 |
| ATOM | 764 | O | ALA | A | 99 | 20.834 | 78.065 | −8.399 | 1.00 | 25.07 |
| ATOM | 765 | N | VAL | A | 100 | 20.547 | 77.040 | −6.420 | 1.00 | 20.36 |
| ATOM | 766 | CA | VAL | A | 100 | 19.106 | 77.274 | −6.388 | 1.00 | 17.58 |
| ATOM | 767 | CB | VAL | A | 100 | 18.462 | 76.528 | −5.196 | 1.00 | 16.99 |
| ATOM | 768 | CG1 | VAL | A | 100 | 16.971 | 76.786 | −5.130 | 1.00 | 14.03 |
| ATOM | 769 | CG2 | VAL | A | 100 | 18.722 | 75.044 | −5.313 | 1.00 | 15.21 |
| ATOM | 770 | C | VAL | A | 100 | 18.797 | 78.764 | −6.277 | 1.00 | 20.86 |
| ATOM | 771 | O | VAL | A | 100 | 19.574 | 79.506 | −5.697 | 1.00 | 25.82 |
| ATOM | 772 | N | GLU | A | 101 | 17.696 | 79.201 | −6.885 | 1.00 | 20.04 |
| ATOM | 773 | CA | GLU | A | 101 | 17.236 | 80.595 | −6.829 | 1.00 | 17.64 |
| ATOM | 774 | CB | GLU | A | 101 | 17.352 | 81.316 | −8.185 | 1.00 | 22.33 |
| ATOM | 775 | CG | GLU | A | 101 | 18.682 | 81.355 | −8.862 | 1.00 | 27.61 |
| ATOM | 776 | CD | GLU | A | 101 | 18.654 | 82.264 | −10.048 | 1.00 | 28.27 |
| ATOM | 777 | OE1 | GLU | A | 101 | 19.711 | 82.803 | −10.393 | 1.00 | 35.84 |
| ATOM | 778 | OE2 | GLU | A | 101 | 17.591 | 82.440 | −10.656 | 1.00 | 33.44 |
| ATOM | 779 | C | GLU | A | 101 | 15.734 | 80.502 | −6.561 | 1.00 | 18.04 |
| ATOM | 780 | O | GLU | A | 101 | 15.085 | 79.542 | −6.977 | 1.00 | 20.27 |
| ATOM | 781 | N | SER | A | 102 | 15.151 | 81.543 | −5.989 | 1.00 | 15.18 |
| ATOM | 782 | CA | SER | A | 102 | 13.727 | 81.512 | −5.749 | 1.00 | 14.63 |
| ATOM | 783 | CB | SER | A | 102 | 13.372 | 80.575 | −4.599 | 1.00 | 16.17 |
| ATOM | 784 | OG | SER | A | 102 | 14.095 | 80.881 | −3.441 | 1.00 | 19.01 |
| ATOM | 785 | C | SER | A | 102 | 13.172 | 82.883 | −5.512 | 1.00 | 14.90 |
| ATOM | 786 | O | SER | A | 102 | 13.919 | 83.805 | −5.226 | 1.00 | 16.69 |
| ATOM | 787 | N | PHE | A | 103 | 11.871 | 83.018 | −5.753 | 1.00 | 16.08 |
| ATOM | 788 | CA | PHE | A | 103 | 11.135 | 84.261 | −5.579 | 1.00 | 14.05 |
| ATOM | 789 | CB | PHE | A | 103 | 10.561 | 84.710 | −6.928 | 1.00 | 11.82 |
| ATOM | 790 | CG | PHE | A | 103 | 9.644 | 85.913 | −6.849 | 1.00 | 14.83 |
| ATOM | 791 | CD1 | PHE | A | 103 | 10.149 | 87.189 | −6.601 | 1.00 | 16.95 |
| ATOM | 792 | CD2 | PHE | A | 103 | 8.279 | 85.773 | −7.081 | 1.00 | 11.21 |
| ATOM | 793 | CE1 | PHE | A | 103 | 9.295 | 88.309 | −6.583 | 1.00 | 12.90 |
| ATOM | 794 | CE2 | PHE | A | 103 | 7.425 | 86.879 | −7.065 | 1.00 | 10.94 |
| ATOM | 795 | CZ | PHE | A | 103 | 7.936 | 88.146 | −6.820 | 1.00 | 12.78 |
| ATOM | 796 | C | PHE | A | 103 | 10.004 | 83.930 | −4.607 | 1.00 | 14.85 |
| ATOM | 797 | O | PHE | A | 103 | 9.249 | 82.981 | −4.822 | 1.00 | 14.06 |
| ATOM | 798 | N | CYS | A | 104 | 9.887 | 84.710 | −3.543 | 1.00 | 14.45 |
| ATOM | 799 | CA | CYS | A | 104 | 8.853 | 84.484 | −2.548 | 1.00 | 14.61 |
| ATOM | 800 | CB | CYS | A | 104 | 9.509 | 84.235 | −1.194 | 1.00 | 14.79 |
| ATOM | 801 | SG | CYS | A | 104 | 8.386 | 84.134 | 0.215 | 1.00 | 18.12 |
| ATOM | 802 | C | CYS | A | 104 | 7.896 | 85.662 | −2.429 | 1.00 | 18.09 |
| ATOM | 803 | O | CYS | A | 104 | 8.322 | 86.821 | −2.482 | 1.00 | 18.91 |
| ATOM | 804 | N | TYR | A | 105 | 6.600 | 85.367 | −2.339 | 1.00 | 17.06 |
| ATOM | 805 | CA | TYR | A | 105 | 5.594 | 86.403 | −2.161 | 1.00 | 13.87 |
| ATOM | 806 | CB | TYR | A | 105 | 4.941 | 86.845 | −3.486 | 1.00 | 12.35 |
| ATOM | 807 | CG | TYR | A | 105 | 4.103 | 85.830 | −4.234 | 1.00 | 14.43 |
| ATOM | 808 | CD1 | TYR | A | 105 | 2.713 | 85.826 | −4.110 | 1.00 | 9.55 |
| ATOM | 809 | CE1 | TYR | A | 105 | 1.927 | 84.933 | −4.832 | 1.00 | 7.61 |
| ATOM | 810 | CD2 | TYR | A | 105 | 4.690 | 84.909 | −5.112 | 1.00 | 11.05 |
| ATOM | 811 | CE2 | TYR | A | 105 | 3.906 | 84.010 | −5.846 | 1.00 | 13.52 |
| ATOM | 812 | CZ | TYR | A | 105 | 2.525 | 84.028 | −5.699 | 1.00 | 15.44 |
| ATOM | 813 | OH | TYR | A | 105 | 1.733 | 83.129 | −6.395 | 1.00 | 18.33 |
| ATOM | 814 | C | TYR | A | 105 | 4.576 | 85.967 | −1.100 | 1.00 | 17.62 |
| ATOM | 815 | O | TYR | A | 105 | 4.412 | 84.773 | −0.820 | 1.00 | 14.49 |
| ATOM | 816 | N | LEU | A | 106 | 3.991 | 86.954 | −0.432 | 1.00 | 18.67 |
| ATOM | 817 | CA | LEU | A | 106 | 3.031 | 86.742 | 0.640 | 1.00 | 17.72 |
| ATOM | 818 | CB | LEU | A | 106 | 3.437 | 87.602 | 1.844 | 1.00 | 15.11 |
| ATOM | 819 | CG | LEU | A | 106 | 4.930 | 87.554 | 2.171 | 1.00 | 18.61 |
| ATOM | 820 | CD1 | LEU | A | 106 | 5.237 | 88.376 | 3.384 | 1.00 | 18.40 |
| ATOM | 821 | CD2 | LEU | A | 106 | 5.358 | 86.124 | 2.391 | 1.00 | 20.22 |
| ATOM | 822 | C | LEU | A | 106 | 1.594 | 87.078 | 0.237 | 1.00 | 19.75 |
| ATOM | 823 | O | LEU | A | 106 | 1.266 | 87.191 | −0.947 | 1.00 | 19.55 |
| ATOM | 824 | N | ASN | A | 107 | 0.753 | 87.241 | 1.253 | 1.00 | 19.69 |
| ATOM | 825 | CA | ASN | A | 107 | −0.659 | 87.563 | 1.122 | 1.00 | 16.25 |
| ATOM | 826 | CB | ASN | A | 107 | −1.231 | 87.792 | 2.518 | 1.00 | 18.19 |
| ATOM | 827 | CG | ASN | A | 107 | −2.738 | 87.979 | 2.530 | 1.00 | 24.35 |
| ATOM | 828 | OD1 | ASN | A | 107 | −3.332 | 88.525 | 1.591 | 1.00 | 22.21 |
| ATOM | 829 | ND2 | ASN | A | 107 | −3.362 | 87.551 | 3.618 | 1.00 | 16.86 |
| ATOM | 830 | C | ASN | A | 107 | −0.817 | 88.812 | 0.279 | 1.00 | 15.33 |
| ATOM | 831 | O | ASN | A | 107 | −0.332 | 89.888 | 0.634 | 1.00 | 16.38 |
| ATOM | 832 | N | PRO | A | 108 | −1.497 | 88.686 | −0.860 | 1.00 | 14.14 |
| ATOM | 833 | CD | PRO | A | 108 | −1.973 | 87.430 | −1.466 | 1.00 | 13.05 |
| ATOM | 834 | CA | PRO | A | 108 | −1.712 | 89.818 | −1.757 | 1.00 | 14.52 |
| ATOM | 835 | CB | PRO | A | 108 | −2.552 | 89.206 | −2.867 | 1.00 | 11.78 |
| ATOM | 836 | CG | PRO | A | 108 | −2.018 | 87.779 | −2.916 | 1.00 | 13.10 |
| ATOM | 837 | C | PRO | A | 108 | −2.409 | 91.006 | −1.113 | 1.00 | 15.10 |
| ATOM | 838 | O | PRO | A | 108 | −2.295 | 92.126 | −1.595 | 1.00 | 15.99 |
| ATOM | 839 | N | ASN | A | 109 | −3.114 | 90.776 | −0.014 | 1.00 | 16.27 |
| ATOM | 840 | CA | ASN | A | 109 | −3.838 | 91.855 | 0.649 | 1.00 | 18.12 |
| ATOM | 841 | CB | ASN | A | 109 | −5.005 | 91.304 | 1.461 | 1.00 | 17.89 |
| ATOM | 842 | CG | ASN | A | 109 | −6.058 | 90.695 | 0.590 | 1.00 | 15.82 |
| ATOM | 843 | OD1 | ASN | A | 109 | −6.374 | 91.228 | −0.475 | 1.00 | 18.28 |
| ATOM | 844 | ND2 | ASN | A | 109 | −6.578 | 89.549 | 1.001 | 1.00 | 14.24 |
| ATOM | 845 | C | ASN | A | 109 | −2.990 | 92.759 | 1.511 | 1.00 | 19.06 |
| ATOM | 846 | O | ASN | A | 109 | −3.467 | 93.786 | 1.978 | 1.00 | 22.19 |
| ATOM | 847 | N | PHE | A | 110 | −1.762 | 92.349 | 1.791 | 1.00 | 17.26 |
| ATOM | 848 | CA | PHE | A | 110 | −0.879 | 93.168 | 2.588 | 1.00 | 13.51 |
| ATOM | 849 | CB | PHE | A | 110 | 0.304 | 92.245 | 3.068 | 1.00 | 11.28 |
| ATOM | 850 | CG | PHE | A | 110 | −0.054 | 91.343 | 4.110 | 1.00 | 14.89 |
| ATOM | 851 | CD1 | PHE | A | 110 | −1.296 | 91.384 | 4.727 | 1.00 | 14.80 |
| ATOM | 852 | CD2 | PHE | A | 110 | 0.854 | 90.365 | 4.494 | 1.00 | 16.71 |
| ATOM | 853 | CE1 | PHE | A | 110 | −1.627 | 90.470 | 5.702 | 1.00 | 15.38 |
| ATOM | 854 | CE2 | PHE | A | 110 | 0.532 | 89.440 | 5.474 | 1.00 | 14.69 |
| ATOM | 855 | CZ | PHE | A | 110 | −0.710 | 89.495 | 6.082 | 1.00 | 18.74 |
| ATOM | 856 | C | PHE | A | 110 | −0.404 | 94.363 | 1.787 | 1.00 | 16.88 |
| ATOM | 857 | O | PHE | A | 110 | 0.469 | 94.243 | 0.930 | 1.00 | 21.88 |
| ATOM | 858 | N | THR | A | 111 | −1.032 | 95.509 | 2.004 | 1.00 | 18.68 |
| ATOM | 859 | CA | THR | A | 111 | −0.625 | 96.718 | 1.304 | 1.00 | 16.37 |
| ATOM | 860 | CB | THR | A | 111 | −1.764 | 97.305 | 0.482 | 1.00 | 15.54 |
| ATOM | 861 | OG1 | THR | A | 111 | −2.723 | 97.911 | 1.355 | 1.00 | 18.30 |
| ATOM | 862 | CG2 | THR | A | 111 | −2.423 | 96.221 | −0.337 | 1.00 | 11.84 |
| ATOM | 863 | C | THR | A | 111 | −0.219 | 97.692 | 2.389 | 1.00 | 18.43 |
| ATOM | 864 | O | THR | A | 111 | −0.284 | 97.360 | 3.564 | 1.00 | 23.70 |
| ATOM | 865 | N | PRO | A | 112 | 0.229 | 98.895 | 2.023 | 1.00 | 21.65 |
| ATOM | 866 | CD | PRO | A | 112 | 0.707 | 99.351 | 0.703 | 1.00 | 15.84 |
| ATOM | 867 | CA | PRO | A | 112 | 0.626 | 99.845 | 3.069 | 1.00 | 23.93 |
| ATOM | 868 | CB | PRO | A | 112 | 1.273 | 100.969 | 2.272 | 1.00 | 21.58 |
| ATOM | 869 | CG | PRO | A | 112 | 1.847 | 100.236 | 1.079 | 1.00 | 19.10 |
| ATOM | 870 | C | PRO | A | 112 | −0.507 | 100.371 | 3.954 | 1.00 | 28.77 |
| ATOM | 871 | O | PRO | A | 112 | −0.245 | 100.991 | 4.981 | 1.00 | 34.33 |
| ATOM | 872 | N | ASP | A | 113 | −1.756 | 100.137 | 3.557 | 1.00 | 30.89 |
| ATOM | 873 | CA | ASP | A | 113 | −2.908 | 100.608 | 4.325 | 1.00 | 29.23 |
| ATOM | 874 | CB | ASP | A | 113 | −4.095 | 100.876 | 3.403 | 1.00 | 33.62 |
| ATOM | 875 | CG | ASP | A | 113 | −3.740 | 101.759 | 2.229 | 1.00 | 39.32 |
| ATOM | 876 | OD1 | ASP | A | 113 | −3.551 | 102.980 | 2.420 | 1.00 | 38.82 |
| ATOM | 877 | OD2 | ASP | A | 113 | −3.659 | 101.220 | 1.107 | 1.00 | 41.64 |
| ATOM | 878 | C | ASP | A | 113 | −3.313 | 99.552 | 5.330 | 1.00 | 26.58 |
| ATOM | 879 | O | ASP | A | 113 | −4.020 | 99.831 | 6.288 | 1.00 | 30.47 |
| ATOM | 880 | N | HIS | A | 114 | −2.875 | 98.327 | 5.090 | 1.00 | 22.11 |
| ATOM | 881 | CA | HIS | A | 114 | −3.203 | 97.223 | 5.964 | 1.00 | 18.51 |
| ATOM | 882 | CB | HIS | A | 114 | −2.562 | 95.954 | 5.434 | 1.00 | 18.88 |
| ATOM | 883 | CG | HIS | A | 114 | −3.154 | 94.695 | 5.980 | 1.00 | 20.47 |
| ATOM | 884 | CD2 | HIS | A | 114 | −3.955 | 93.771 | 5.401 | 1.00 | 20.61 |
| ATOM | 885 | ND1 | HIS | A | 114 | −2.905 | 94.243 | 7.255 | 1.00 | 21.89 |
| ATOM | 886 | CE1 | HIS | A | 114 | −3.527 | 93.092 | 7.440 | 1.00 | 20.69 |
| ATOM | 887 | NE2 | HIS | A | 114 | −4.170 | 92.784 | 6.331 | 1.00 | 16.26 |
| ATOM | 888 | C | HIS | A | 114 | −2.682 | 97.529 | 7.347 | 1.00 | 20.91 |
| ATOM | 889 | O | HIS | A | 114 | −1.511 | 97.821 | 7.518 | 1.00 | 23.53 |
| ATOM | 890 | N | PRO | A | 115 | −3.535 | 97.414 | 8.365 | 1.00 | 23.07 |
| ATOM | 891 | CD | PRO | A | 115 | −4.933 | 96.973 | 8.311 | 1.00 | 24.88 |
| ATOM | 892 | CA | PRO | A | 115 | −3.148 | 97.688 | 9.745 | 1.00 | 24.93 |
| ATOM | 893 | CB | PRO | A | 115 | −4.411 | 97.334 | 10.525 | 1.00 | 27.48 |
| ATOM | 894 | CG | PRO | A | 115 | −5.094 | 96.342 | 9.652 | 1.00 | 24.00 |
| ATOM | 895 | C | PRO | A | 115 | −1.927 | 96.923 | 10.233 | 1.00 | 26.06 |
| ATOM | 896 | O | PRO | A | 115 | −1.127 | 97.456 | 10.991 | 1.00 | 32.32 |
| ATOM | 897 | N | ARG | A | 116 | −1.782 | 95.678 | 9.802 | 1.00 | 26.43 |
| ATOM | 898 | CA | ARG | A | 116 | −0.634 | 94.868 | 10.199 | 1.00 | 26.26 |
| ATOM | 899 | CB | ARG | A | 116 | −0.810 | 93.430 | 9.727 | 1.00 | 30.69 |
| ATOM | 900 | CG | ARG | A | 116 | −1.813 | 92.629 | 10.516 | 1.00 | 35.60 |
| ATOM | 901 | CD | ARG | A | 116 | −1.372 | 92.533 | 11.950 | 1.00 | 39.57 |
| ATOM | 902 | NE | ARG | A | 116 | −2.151 | 91.547 | 12.685 | 1.00 | 44.77 |
| ATOM | 903 | CZ | ARG | A | 116 | −2.021 | 91.314 | 13.988 | 1.00 | 47.07 |
| ATOM | 904 | NH1 | ARG | A | 116 | −1.145 | 91.997 | 14.721 | 1.00 | 44.22 |
| ATOM | 905 | NH2 | ARG | A | 116 | −2.758 | 90.379 | 14.556 | 1.00 | 46.39 |
| ATOM | 906 | C | ARG | A | 116 | 0.681 | 95.416 | 9.653 | 1.00 | 27.56 |
| ATOM | 907 | O | ARG | A | 116 | 1.727 | 95.273 | 10.276 | 1.00 | 28.41 |
| ATOM | 908 | N | ILE | A | 117 | 0.624 | 96.021 | 8.473 | 1.00 | 28.17 |
| ATOM | 909 | CA | ILE | A | 117 | 1.806 | 96.580 | 7.835 | 1.00 | 28.31 |
| ATOM | 910 | CB | ILE | A | 117 | 1.584 | 96.734 | 6.307 | 1.00 | 24.00 |
| ATOM | 911 | CG2 | ILE | A | 117 | 2.790 | 97.357 | 5.644 | 1.00 | 23.25 |
| ATOM | 912 | CG1 | ILE | A | 117 | 1.315 | 95.372 | 5.668 | 1.00 | 20.98 |
| ATOM | 913 | CD1 | ILE | A | 117 | 2.506 | 94.482 | 5.596 | 1.00 | 16.97 |
| ATOM | 914 | C | ILE | A | 117 | 2.140 | 97.930 | 8.490 | 1.00 | 34.06 |
| ATOM | 915 | O | ILE | A | 117 | 3.308 | 98.237 | 8.742 | 1.00 | 40.01 |
| ATOM | 916 | N | GLN | A | 118 | 1.111 | 98.716 | 8.797 | 1.00 | 34.32 |
| ATOM | 917 | CA | GLN | A | 118 | 1.289 | 100.008 | 9.446 | 1.00 | 31.25 |
| ATOM | 918 | CB | GLN | A | 118 | −0.036 | 100.751 | 9.510 | 1.00 | 32.56 |
| ATOM | 919 | CG | GLN | A | 118 | −0.532 | 101.215 | 8.175 | 1.00 | 41.97 |
| ATOM | 920 | CD | GLN | A | 118 | −1.790 | 102.040 | 8.289 | 1.00 | 49.04 |
| ATOM | 921 | OE1 | GLN | A | 118 | −2.349 | 102.203 | 9.374 | 1.00 | 54.55 |
| ATOM | 922 | NE2 | GLN | A | 118 | −2.241 | 102.577 | 7.167 | 1.00 | 54.77 |
| ATOM | 923 | C | GLN | A | 118 | 1.813 | 99.816 | 10.857 | 1.00 | 30.00 |
| ATOM | 924 | O | GLN | A | 118 | 2.606 | 100.602 | 11.348 | 1.00 | 36.36 |
| ATOM | 925 | N | ALA | A | 119 | 1.362 | 98.759 | 11.506 | 1.00 | 27.04 |
| ATOM | 926 | CA | ALA | A | 119 | 1.801 | 98.470 | 12.851 | 1.00 | 23.94 |
| ATOM | 927 | CB | ALA | A | 119 | 0.898 | 97.447 | 13.457 | 1.00 | 23.97 |
| ATOM | 928 | C | ALA | A | 119 | 3.223 | 97.944 | 12.836 | 1.00 | 29.31 |
| ATOM | 929 | O | ALA | A | 119 | 3.839 | 97.802 | 13.885 | 1.00 | 35.94 |
| ATOM | 930 | N | LYS | A | 120 | 3.728 | 97.625 | 11.649 | 1.00 | 30.68 |
| ATOM | 931 | CA | LYS | A | 120 | 5.068 | 97.075 | 11.497 | 1.00 | 31.26 |
| ATOM | 932 | CB | LYS | A | 120 | 6.131 | 98.127 | 11.815 | 1.00 | 32.35 |
| ATOM | 933 | CG | LYS | A | 120 | 6.210 | 99.208 | 10.756 | 1.00 | 41.07 |
| ATOM | 934 | CD | LYS | A | 120 | 7.461 | 100.047 | 10.893 | 1.00 | 53.39 |
| ATOM | 935 | CE | LYS | A | 120 | 7.720 | 100.864 | 9.621 | 1.00 | 60.71 |
| ATOM | 936 | NZ | LYS | A | 120 | 9.046 | 101.563 | 9.644 | 1.00 | 65.99 |
| ATOM | 937 | C | LYS | A | 120 | 5.271 | 95.796 | 12.321 | 1.00 | 30.21 |
| ATOM | 938 | O | LYS | A | 120 | 6.266 | 95.633 | 13.014 | 1.00 | 34.12 |
| ATOM | 939 | N | THR | A | 121 | 4.287 | 94.901 | 12.240 | 1.00 | 30.59 |
| ATOM | 940 | CA | THR | A | 121 | 4.304 | 93.627 | 12.945 | 1.00 | 29.59 |
| ATOM | 941 | CB | THR | A | 121 | 2.897 | 93.001 | 12.953 | 1.00 | 26.01 |
| ATOM | 942 | OG1 | THR | A | 121 | 1.948 | 93.993 | 13.347 | 1.00 | 29.93 |
| ATOM | 943 | CG2 | THR | A | 121 | 2.830 | 91.841 | 13.924 | 1.00 | 27.80 |
| ATOM | 944 | C | THR | A | 121 | 5.238 | 92.685 | 12.203 | 1.00 | 31.22 |
| ATOM | 945 | O | THR | A | 121 | 5.258 | 92.684 | 10.977 | 1.00 | 36.88 |
| ATOM | 946 | N | PRO | A | 122 | 6.016 | 91.867 | 12.918 | 1.00 | 31.48 |
| ATOM | 947 | CD | PRO | A | 122 | 6.173 | 91.788 | 14.375 | 1.00 | 30.51 |
| ATOM | 948 | CA | PRO | A | 122 | 6.936 | 90.939 | 12.260 | 1.00 | 29.39 |
| ATOM | 949 | CB | PRO | A | 122 | 7.519 | 90.162 | 13.428 | 1.00 | 28.00 |
| ATOM | 950 | CG | PRO | A | 122 | 7.524 | 91.147 | 14.508 | 1.00 | 28.89 |
| ATOM | 951 | C | PRO | A | 122 | 6.213 | 89.993 | 11.324 | 1.00 | 29.95 |
| ATOM | 952 | O | PRO | A | 122 | 5.017 | 89.759 | 11.499 | 1.00 | 33.22 |
| ATOM | 953 | N | THR | A | 123 | 6.957 | 89.422 | 10.373 | 1.00 | 30.79 |
| ATOM | 954 | CA | THR | A | 123 | 6.470 | 88.472 | 9.355 | 1.00 | 28.32 |
| ATOM | 955 | CB | THR | A | 123 | 5.859 | 87.189 | 9.961 | 1.00 | 30.03 |
| ATOM | 956 | OG1 | THR | A | 123 | 4.648 | 87.498 | 10.661 | 1.00 | 35.13 |
| ATOM | 957 | CG2 | THR | A | 123 | 6.835 | 86.507 | 10.897 | 1.00 | 31.48 |
| ATOM | 958 | C | THR | A | 123 | 5.505 | 89.028 | 8.309 | 1.00 | 27.79 |
| ATOM | 959 | O | THR | A | 123 | 5.164 | 88.310 | 7.377 | 1.00 | 29.06 |
| ATOM | 960 | N | HIS | A | 124 | 5.076 | 90.285 | 8.458 | 1.00 | 24.80 |
| ATOM | 961 | CA | HIS | A | 124 | 4.150 | 90.920 | 7.504 | 1.00 | 22.12 |
| ATOM | 962 | CB | HIS | A | 124 | 3.072 | 91.740 | 8.226 | 1.00 | 17.07 |
| ATOM | 963 | CG | HIS | A | 124 | 2.285 | 90.948 | 9.215 | 1.00 | 13.05 |
| ATOM | 964 | CD2 | HIS | A | 124 | 1.035 | 90.435 | 9.145 | 1.00 | 18.78 |
| ATOM | 965 | ND1 | HIS | A | 124 | 2.798 | 90.529 | 10.420 | 1.00 | 15.62 |
| ATOM | 966 | CE1 | HIS | A | 124 | 1.907 | 89.785 | 11.044 | 1.00 | 17.88 |
| ATOM | 967 | NE2 | HIS | A | 124 | 0.825 | 89.712 | 10.292 | 1.00 | 17.44 |
| ATOM | 968 | C | HIS | A | 124 | 4.941 | 91.837 | 6.592 | 1.00 | 21.71 |
| ATOM | 969 | O | HIS | A | 124 | 5.645 | 92.715 | 7.050 | 1.00 | 22.54 |
| ATOM | 970 | N | GLU | A | 125 | 4.819 | 91.632 | 5.295 | 1.00 | 21.78 |
| ATOM | 971 | CA | GLU | A | 125 | 5.538 | 92.456 | 4.339 | 1.00 | 20.92 |
| ATOM | 972 | CB | GLU | A | 125 | 6.787 | 91.730 | 3.852 | 1.00 | 23.07 |
| ATOM | 973 | CG | GLU | A | 125 | 8.023 | 91.998 | 4.651 | 1.00 | 24.04 |
| ATOM | 974 | CD | GLU | A | 125 | 9.169 | 91.132 | 4.222 | 1.00 | 24.79 |
| ATOM | 975 | OE1 | GLU | A | 125 | 9.457 | 91.049 | 3.012 | 1.00 | 24.03 |
| ATOM | 976 | OE2 | GLU | A | 125 | 9.793 | 90.539 | 5.107 | 1.00 | 31.23 |
| ATOM | 977 | C | GLU | A | 125 | 4.668 | 92.738 | 3.143 | 1.00 | 21.05 |
| ATOM | 978 | O | GLU | A | 125 | 3.660 | 92.080 | 2.930 | 1.00 | 24.62 |
| ATOM | 979 | N | VAL | A | 126 | 5.091 | 93.694 | 2.339 | 1.00 | 21.65 |
| ATOM | 980 | CA | VAL | A | 126 | 4.361 | 94.031 | 1.146 | 1.00 | 22.78 |
| ATOM | 981 | CB | VAL | A | 126 | 4.203 | 95.538 | 1.042 | 1.00 | 20.08 |
| ATOM | 982 | CG1 | VAL | A | 126 | 3.508 | 95.900 | −0.229 | 1.00 | 17.38 |
| ATOM | 983 | CG2 | VAL | A | 126 | 3.405 | 96.028 | 2.228 | 1.00 | 20.49 |
| ATOM | 984 | C | VAL | A | 126 | 5.166 | 93.459 | −0.019 | 1.00 | 25.89 |
| ATOM | 985 | O | VAL | A | 126 | 6.380 | 93.667 | −0.099 | 1.00 | 30.48 |
| ATOM | 986 | N | ASN | A | 127 | 4.511 | 92.683 | −0.880 | 1.00 | 21.77 |
| ATOM | 987 | CA | ASN | A | 127 | 5.174 | 92.053 | −2.020 | 1.00 | 19.25 |
| ATOM | 988 | CB | ASN | A | 127 | 4.182 | 91.207 | −2.830 | 1.00 | 18.25 |
| ATOM | 989 | CG | ASN | A | 127 | 3.724 | 89.985 | −2.095 | 1.00 | 16.94 |
| ATOM | 990 | OD1 | ASN | A | 127 | 4.459 | 89.445 | −1.286 | 1.00 | 16.08 |
| ATOM | 991 | ND2 | ASN | A | 127 | 2.512 | 89.528 | −2.380 | 1.00 | 12.62 |
| ATOM | 992 | C | ASN | A | 127 | 5.857 | 93.007 | −2.975 | 1.00 | 19.23 |
| ATOM | 993 | O | ASN | A | 127 | 5.436 | 94.150 | −3.148 | 1.00 | 18.59 |
| ATOM | 994 | N | VAL | A | 128 | 6.948 | 92.525 | −3.556 | 1.00 | 18.95 |
| ATOM | 995 | CA | VAL | A | 128 | 7.698 | 93.260 | −4.561 | 1.00 | 23.17 |
| ATOM | 996 | CB | VAL | A | 128 | 9.228 | 93.403 | −4.235 | 1.00 | 21.82 |
| ATOM | 997 | CG1 | VAL | A | 128 | 9.427 | 94.284 | −3.027 | 1.00 | 20.58 |
| ATOM | 998 | CG2 | VAL | A | 128 | 9.880 | 92.055 | −3.999 | 1.00 | 23.71 |
| ATOM | 999 | C | VAL | A | 128 | 7.494 | 92.408 | −5.808 | 1.00 | 23.82 |
| ATOM | 1000 | O | VAL | A | 128 | 7.486 | 91.183 | −5.730 | 1.00 | 23.35 |
| ATOM | 1001 | N | TRP | A | 129 | 7.312 | 93.050 | −6.951 | 1.00 | 25.96 |
| ATOM | 1002 | CA | TRP | A | 129 | 7.066 | 92.319 | −8.179 | 1.00 | 28.04 |
| ATOM | 1003 | CB | TRP | A | 129 | 5.604 | 92.476 | −8.562 | 1.00 | 22.71 |
| ATOM | 1004 | CG | TRP | A | 129 | 4.646 | 91.925 | −7.588 | 1.00 | 20.42 |
| ATOM | 1005 | CD2 | TRP | A | 129 | 4.254 | 90.557 | −7.467 | 1.00 | 17.81 |
| ATOM | 1006 | CE2 | TRP | A | 129 | 3.222 | 90.505 | −6.508 | 1.00 | 16.80 |
| ATOM | 1007 | CE3 | TRP | A | 129 | 4.667 | 89.371 | −8.084 | 1.00 | 17.14 |
| ATOM | 1008 | CD1 | TRP | A | 129 | 3.873 | 92.629 | −6.710 | 1.00 | 15.97 |
| ATOM | 1009 | NE1 | TRP | A | 129 | 3.008 | 91.784 | −6.063 | 1.00 | 17.97 |
| ATOM | 1010 | CZ2 | TRP | A | 129 | 2.597 | 89.313 | −6.155 | 1.00 | 19.47 |
| ATOM | 1011 | CZ3 | TRP | A | 129 | 4.048 | 88.188 | −7.734 | 1.00 | 16.93 |
| ATOM | 1012 | CH2 | TRP | A | 129 | 3.024 | 88.167 | −6.780 | 1.00 | 18.76 |
| ATOM | 1013 | C | TRP | A | 129 | 7.887 | 92.838 | −9.336 | 1.00 | 30.23 |
| ATOM | 1014 | O | TRP | A | 129 | 8.323 | 93.985 | −9.325 | 1.00 | 30.92 |
| ATOM | 1015 | N | PRO | A | 130 | 8.103 | 92.000 | −10.361 | 1.00 | 32.08 |
| ATOM | 1016 | CD | PRO | A | 130 | 7.807 | 90.562 | −10.498 | 1.00 | 31.53 |
| ATOM | 1017 | CA | PRO | A | 130 | 8.880 | 92.474 | −11.503 | 1.00 | 33.41 |
| ATOM | 1018 | CB | PRO | A | 130 | 9.109 | 91.192 | −12.308 | 1.00 | 31.40 |
| ATOM | 1019 | CG | PRO | A | 130 | 7.926 | 90.364 | −11.986 | 1.00 | 28.64 |
| ATOM | 1020 | C | PRO | A | 130 | 8.090 | 93.521 | −12.298 | 1.00 | 35.25 |
| ATOM | 1021 | O | PRO | A | 130 | 6.869 | 93.651 | −12.146 | 1.00 | 36.95 |
| ATOM | 1022 | N | ASP | A | 131 | 8.805 | 94.301 | −13.100 | 1.00 | 38.98 |
| ATOM | 1023 | CA | ASP | A | 131 | 8.185 | 95.329 | −13.927 | 1.00 | 41.94 |
| ATOM | 1024 | CB | ASP | A | 131 | 9.270 | 96.081 | −14.699 | 1.00 | 49.89 |
| ATOM | 1025 | CG | ASP | A | 131 | 8.713 | 97.168 | −15.612 | 1.00 | 59.35 |
| ATOM | 1026 | OD1 | ASP | A | 131 | 7.519 | 97.540 | −15.503 | 1.00 | 60.33 |
| ATOM | 1027 | OD2 | ASP | A | 131 | 9.499 | 97.667 | −16.448 | 1.00 | 69.02 |
| ATOM | 1028 | C | ASP | A | 131 | 7.160 | 94.732 | −14.895 | 1.00 | 40.13 |
| ATOM | 1029 | O | ASP | A | 131 | 7.444 | 93.761 | −15.604 | 1.00 | 39.39 |
| ATOM | 1030 | N | GLU | A | 132 | 5.983 | 95.343 | −14.933 | 1.00 | 38.57 |
| ATOM | 1031 | CA | GLU | A | 132 | 4.895 | 94.906 | −15.796 | 1.00 | 41.66 |
| ATOM | 1032 | CB | GLU | A | 132 | 3.675 | 95.805 | −15.594 | 1.00 | 49.43 |
| ATOM | 1033 | CG | GLU | A | 132 | 2.565 | 95.210 | −14.742 | 1.00 | 63.43 |
| ATOM | 1034 | CD | GLU | A | 132 | 1.767 | 94.123 | −15.455 | 1.00 | 68.77 |
| ATOM | 1035 | OE1 | GLU | A | 132 | 1.390 | 93.130 | −14.786 | 1.00 | 73.49 |
| ATOM | 1036 | OE2 | GLU | A | 132 | 1.501 | 94.268 | −16.671 | 1.00 | 70.44 |
| ATOM | 1037 | C | GLU | A | 132 | 5.249 | 94.919 | −17.269 | 1.00 | 40.84 |
| ATOM | 1038 | O | GLU | A | 132 | 4.925 | 93.984 | −17.998 | 1.00 | 42.27 |
| ATOM | 1039 | N | THR | A | 133 | 5.909 | 95.984 | −17.709 | 1.00 | 40.91 |
| ATOM | 1040 | CA | THR | A | 133 | 6.261 | 96.124 | −19.114 | 1.00 | 42.12 |
| ATOM | 1041 | CB | THR | A | 133 | 7.100 | 97.407 | −19.373 | 1.00 | 43.93 |
| ATOM | 1042 | OG1 | THR | A | 133 | 8.431 | 97.217 | −18.884 | 1.00 | 56.48 |
| ATOM | 1043 | CG2 | THR | A | 133 | 6.496 | 98.602 | −18.650 | 1.00 | 42.47 |
| ATOM | 1044 | C | THR | A | 133 | 7.045 | 94.908 | −19.576 | 1.00 | 40.83 |
| ATOM | 1045 | O | THR | A | 133 | 6.800 | 94.369 | −20.650 | 1.00 | 45.82 |
| ATOM | 1046 | N | LYS | A | 134 | 7.945 | 94.450 | −18.717 | 1.00 | 40.62 |
| ATOM | 1047 | CA | LYS | A | 134 | 8.799 | 93.312 | −19.014 | 1.00 | 40.68 |
| ATOM | 1048 | CB | LYS | A | 134 | 10.109 | 93.429 | −18.233 | 1.00 | 44.31 |
| ATOM | 1049 | CG | LYS | A | 134 | 10.852 | 94.742 | −18.437 | 1.00 | 50.92 |
| ATOM | 1050 | CD | LYS | A | 134 | 12.171 | 94.754 | −17.681 | 1.00 | 56.36 |
| ATOM | 1051 | CE | LYS | A | 134 | 12.883 | 96.096 | −17.809 | 1.00 | 64.21 |
| ATOM | 1052 | NZ | LYS | A | 134 | 14.159 | 96.104 | −17.026 | 1.00 | 72.09 |
| ATOM | 1053 | C | LYS | A | 134 | 8.176 | 91.950 | −18.737 | 1.00 | 40.31 |
| ATOM | 1054 | O | LYS | A | 134 | 8.563 | 90.955 | −19.348 | 1.00 | 45.00 |
| ATOM | 1055 | N | HIS | A | 135 | 7.278 | 91.875 | −17.763 | 1.00 | 38.10 |
| ATOM | 1056 | CA | HIS | A | 135 | 6.649 | 90.601 | −17.425 | 1.00 | 34.82 |
| ATOM | 1057 | CB | HIS | A | 135 | 7.200 | 90.071 | −16.089 | 1.00 | 28.65 |
| ATOM | 1058 | CG | HIS | A | 135 | 8.654 | 89.705 | −16.124 | 1.00 | 21.40 |
| ATOM | 1059 | CD2 | HIS | A | 135 | 9.267 | 88.523 | −16.373 | 1.00 | 22.41 |
| ATOM | 1060 | ND1 | HIS | A | 135 | 9.659 | 90.614 | −15.880 | 1.00 | 21.96 |
| ATOM | 1061 | CE1 | HIS | A | 135 | 10.832 | 90.012 | −15.978 | 1.00 | 21.23 |
| ATOM | 1062 | NE2 | HIS | A | 135 | 10.624 | 88.743 | −16.276 | 1.00 | 26.04 |
| ATOM | 1063 | C | HIS | A | 135 | 5.135 | 90.787 | −17.335 | 1.00 | 37.00 |
| ATOM | 1064 | O | HIS | A | 135 | 4.536 | 90.584 | −16.271 | 1.00 | 36.99 |
| ATOM | 1065 | N | PRO | A | 136 | 4.489 | 91.128 | −18.465 | 1.00 | 38.00 |
| ATOM | 1066 | CD | PRO | A | 136 | 5.065 | 91.093 | −19.820 | 1.00 | 39.72 |
| ATOM | 1067 | CA | PRO | A | 136 | 3.043 | 91.352 | −18.545 | 1.00 | 35.57 |
| ATOM | 1068 | CB | PRO | A | 136 | 2.801 | 91.478 | −20.048 | 1.00 | 33.63 |
| ATOM | 1069 | CG | PRO | A | 136 | 3.882 | 90.651 | −20.639 | 1.00 | 35.65 |
| ATOM | 1070 | C | PRO | A | 136 | 2.183 | 90.264 | −17.935 | 1.00 | 33.54 |
| ATOM | 1071 | O | PRO | A | 136 | 2.348 | 89.083 | −18.231 | 1.00 | 35.53 |
| ATOM | 1072 | N | GLY | A | 137 | 1.284 | 90.680 | −17.053 | 1.00 | 33.66 |
| ATOM | 1073 | CA | GLY | A | 137 | 0.379 | 89.753 | −16.408 | 1.00 | 35.45 |
| ATOM | 1074 | C | GLY | A | 137 | 0.946 | 88.810 | −15.361 | 1.00 | 36.34 |
| ATOM | 1075 | O | GLY | A | 137 | 0.212 | 87.981 | −14.829 | 1.00 | 41.58 |
| ATOM | 1076 | N | PHE | A | 138 | 2.224 | 86.926 | −15.029 | 1.00 | 34.29 |
| ATOM | 1077 | CA | PHE | A | 138 | 2.792 | 88.034 | −14.022 | 1.00 | 32.30 |
| ATOM | 1078 | CB | PHE | A | 138 | 4.315 | 88.175 | −13.960 | 1.00 | 33.36 |
| ATOM | 1079 | CG | PHE | A | 138 | 4.960 | 87.272 | −12.959 | 1.00 | 29.73 |
| ATOM | 1080 | CD1 | PHE | A | 138 | 4.770 | 85.902 | −13.026 | 1.00 | 31.50 |
| ATOM | 1081 | CD2 | PHE | A | 138 | 5.718 | 87.796 | −11.924 | 1.00 | 30.02 |
| ATOM | 1082 | CE1 | PHE | A | 138 | 5.322 | 85.061 | −12.070 | 1.00 | 32.69 |
| ATOM | 1083 | CE2 | PHE | A | 138 | 6.272 | 86.974 | −10.965 | 1.00 | 30.29 |
| ATOM | 1084 | CZ | PHE | A | 138 | 6.075 | 85.600 | −11.035 | 1.00 | 33.63 |
| ATOM | 1085 | C | PHE | A | 138 | 2.178 | 88.259 | −12.639 | 1.00 | 31.05 |
| ATOM | 1086 | O | PHE | A | 138 | 1.653 | 87.323 | −12.029 | 1.00 | 29.39 |
| ATOM | 1087 | N | GLN | A | 139 | 2.205 | 89.502 | −12.169 | 1.00 | 26.99 |
| ATOM | 1088 | CA | GLN | A | 139 | 1.662 | 89.816 | −10.861 | 1.00 | 28.51 |
| ATOM | 1089 | CB | GLN | A | 139 | 1.751 | 91.314 | −10.581 | 1.00 | 23.97 |
| ATOM | 1090 | CG | GLN | A | 139 | 1.090 | 91.725 | −9.275 | 1.00 | 27.92 |
| ATOM | 1091 | CD | GLN | A | 139 | 1.192 | 93.216 | −8.982 | 1.00 | 32.88 |
| ATOM | 1092 | OE1 | GLN | A | 139 | 1.943 | 93.950 | −9.632 | 1.00 | 32.97 |
| ATOM | 1093 | NE2 | GLN | A | 139 | 0.440 | 93.668 | −7.987 | 1.00 | 31.33 |
| ATOM | 1094 | C | GLN | A | 139 | 0.222 | 89.332 | −10.734 | 1.00 | 33.84 |
| ATOM | 1095 | O | GLN | A | 139 | −0.126 | 88.663 | −9.754 | 1.00 | 36.68 |
| ATOM | 1096 | N | ASP | A | 140 | −0.592 | 89.609 | −11.752 | 1.00 | 37.09 |
| ATOM | 1097 | CA | ASP | A | 140 | −2.003 | 89.206 | −11.747 | 1.00 | 35.96 |
| ATOM | 1098 | CB | ASP | A | 140 | −2.736 | 89.776 | −12.972 | 1.00 | 40.62 |
| ATOM | 1099 | CG | ASP | A | 140 | −2.672 | 91.300 | −13.044 | 1.00 | 49.63 |
| ATOM | 1100 | OD1 | ASP | A | 140 | −3.391 | 91.955 | −12.266 | 1.00 | 51.54 |
| ATOM | 1101 | OD2 | ASP | A | 140 | −1.906 | 91.840 | −13.875 | 1.00 | 56.38 |
| ATOM | 1102 | C | ASP | A | 140 | −2.140 | 87.684 | −11.694 | 1.00 | 34.15 |
| ATOM | 1103 | O | ASP | A | 140 | −3.024 | 87.149 | −11.023 | 1.00 | 35.48 |
| ATOM | 1104 | N | PHE | A | 141 | −1.258 | 86.981 | −12.389 | 1.00 | 29.30 |
| ATOM | 1105 | CA | PHE | A | 141 | −1.305 | 85.530 | −12.373 | 1.00 | 29.22 |
| ATOM | 1106 | CB | PHE | A | 141 | −0.296 | 84.942 | −13.368 | 1.00 | 26.63 |
| ATOM | 1107 | CD | PHE | A | 141 | 0.113 | 83.526 | −13.051 | 1.00 | 32.93 |
| ATOM | 1108 | CD1 | PHE | A | 141 | −0.721 | 82.456 | −13.364 | 1.00 | 37.47 |
| ATOM | 1109 | CD2 | PHE | A | 141 | 1.312 | 83.263 | −12.388 | 1.00 | 36.97 |
| ATOM | 1110 | CE1 | PHE | A | 141 | −0.373 | 81.143 | −13.016 | 1.00 | 37.55 |
| ATOM | 1111 | CE2 | PHE | A | 141 | 1.667 | 81.956 | −12.035 | 1.00 | 37.91 |
| ATOM | 1112 | CZ | PHE | A | 141 | 0.821 | 80.895 | −12.349 | 1.00 | 36.70 |
| ATOM | 1113 | C | PHE | A | 141 | −0.971 | 85.023 | −10.969 | 1.00 | 31.10 |
| ATOM | 1114 | O | PHE | A | 141 | −1.720 | 84.264 | −10.369 | 1.00 | 34.39 |
| ATOM | 1115 | N | ALA | A | 142 | 0.162 | 85.493 | −10.454 | 1.00 | 27.56 |
| ATOM | 1116 | CA | ALA | A | 142 | 0.657 | 85.091 | −9.149 | 1.00 | 23.24 |
| ATOM | 1117 | CB | ALA | A | 142 | 1.969 | 85.792 | −8.880 | 1.00 | 26.02 |
| ATOM | 1118 | C | ALA | A | 142 | −0.317 | 85.313 | −7.995 | 1.00 | 23.50 |
| ATOM | 1119 | O | ALA | A | 142 | −0.438 | 84.480 | −7.094 | 1.00 | 23.30 |
| ATOM | 1120 | N | GLU | A | 143 | −1.013 | 86.437 | −8.022 | 1.00 | 24.11 |
| ATOM | 1121 | CA | GLU | A | 143 | −1.969 | 86.750 | −6.970 | 1.00 | 26.83 |
| ATOM | 1122 | CB | GLU | A | 143 | −2.403 | 88.211 | −7.077 | 1.00 | 27.46 |
| ATOM | 1123 | CG | GLU | A | 143 | −1.262 | 89.196 | −6.890 | 1.00 | 30.94 |
| ATOM | 1124 | CD | GLU | A | 143 | −1.733 | 90.618 | −6.681 | 1.00 | 36.67 |
| ATOM | 1125 | OE1 | GLU | A | 143 | −0.906 | 91.448 | −6.250 | 1.00 | 40.24 |
| ATOM | 1126 | OE2 | GLU | A | 143 | −2.921 | 90.916 | −6.943 | 1.00 | 40.10 |
| ATOM | 1127 | C | GLU | A | 143 | −3.183 | 85.824 | −7.018 | 1.00 | 29.25 |
| ATOM | 1128 | O | GLU | A | 143 | −3.640 | 85.318 | −5.989 | 1.00 | 31.43 |
| ATOM | 1129 | N | GLN | A | 144 | −3.699 | 85.596 | −8.219 | 1.00 | 30.46 |
| ATOM | 1130 | CA | GLN | A | 144 | −4.843 | 84.725 | −8.392 | 1.00 | 28.86 |
| ATOM | 1131 | CB | GLN | A | 144 | −5.275 | 84.696 | −9.858 | 1.00 | 38.80 |
| ATOM | 1132 | CG | GLN | A | 144 | −6.529 | 83.852 | −10.139 | 1.00 | 58.07 |
| ATOM | 1133 | CD | GLN | A | 144 | −7.754 | 84.269 | −9.309 | 1.00 | 67.99 |
| ATOM | 1134 | OE1 | GLN | A | 144 | −8.542 | 83.420 | −8.876 | 1.00 | 72.49 |
| ATOM | 1135 | NE2 | GLN | A | 144 | −7.922 | 85.573 | −9.099 | 1.00 | 74.56 |
| ATOM | 1136 | C | GLN | A | 144 | −4.449 | 83.339 | −7.938 | 1.00 | 25.81 |
| ATOM | 1137 | O | GLN | A | 144 | −5.201 | 82.669 | −7.239 | 1.00 | 30.61 |
| ATOM | 1138 | N | TYR | A | 145 | −3.259 | 82.905 | −8.335 | 1.00 | 26.62 |
| ATOM | 1139 | CA | TYR | A | 145 | −2.788 | 81.590 | −7.944 | 1.00 | 22.39 |
| ATOM | 1140 | CB | TYR | A | 145 | −1.393 | 81.286 | −8.505 | 1.00 | 21.02 |
| ATOM | 1141 | CG | TYR | A | 145 | −0.895 | 79.930 | −8.047 | 1.00 | 23.34 |
| ATOM | 1142 | CD1 | TYR | A | 145 | −1.603 | 78.770 | −8.356 | 1.00 | 21.49 |
| ATOM | 1143 | CE1 | TYR | A | 145 | −1.224 | 77.533 | −7.846 | 1.00 | 19.67 |
| ATOM | 1144 | CD2 | TYR | A | 145 | 0.221 | 79.814 | −7.219 | 1.00 | 23.57 |
| ATOM | 1145 | CE2 | TYR | A | 145 | 0.607 | 78.579 | −6.705 | 1.00 | 20.74 |
| ATOM | 1146 | CZ | TYR | A | 145 | −0.122 | 77.449 | −7.024 | 1.00 | 20.82 |
| ATOM | 1147 | OH | TYR | A | 145 | 0.250 | 76.231 | −6.518 | 1.00 | 24.24 |
| ATOM | 1148 | C | TYR | A | 145 | −2.791 | 81.487 | −6.422 | 1.00 | 22.08 |
| ATOM | 1149 | O | TYR | A | 145 | −3.231 | 80.482 | −5.875 | 1.00 | 24.79 |
| ATOM | 1150 | N | TYR | A | 146 | −2.360 | 82.546 | −5.740 | 1.00 | 20.10 |
| ATOM | 1151 | CA | TYR | A | 146 | −2.329 | 82.552 | −4.283 | 1.00 | 16.74 |
| ATOM | 1152 | CB | TYR | A | 146 | −1.933 | 83.931 | −3.773 | 1.00 | 15.16 |
| ATOM | 1153 | CG | TYR | A | 146 | −1.652 | 83.991 | −2.289 | 1.00 | 19.44 |
| ATOM | 1154 | CD1 | TYR | A | 146 | −0.345 | 84.050 | −1.807 | 1.00 | 19.08 |
| ATOM | 1155 | CE1 | TYR | A | 146 | −0.088 | 84.132 | −0.458 | 1.00 | 17.67 |
| ATOM | 1156 | CD2 | TYR | A | 146 | −2.691 | 84.023 | −1.364 | 1.00 | 18.53 |
| ATOM | 1157 | CE2 | TYR | A | 146 | −2.438 | 84.103 | −0.002 | 1.00 | 17.70 |
| ATOM | 1158 | CZ | TYR | A | 146 | −1.137 | 84.161 | 0.437 | 1.00 | 19.52 |
| ATOM | 1159 | OH | TYR | A | 146 | −0.875 | 84.226 | 1.774 | 1.00 | 25.01 |
| ATOM | 1160 | C | TYR | A | 146 | −3.704 | 82.193 | −3.770 | 1.00 | 17.31 |
| ATOM | 1161 | O | TYR | A | 146 | −3.859 | 81.274 | −2.967 | 1.00 | 21.09 |
| ATOM | 1162 | N | TRP | A | 147 | −4.713 | 82.882 | −4.284 | 1.00 | 17.15 |
| ATOM | 1163 | CA | TRP | A | 147 | −6.087 | 82.620 | −3.881 | 1.00 | 17.97 |
| ATOM | 1164 | CB | TRP | A | 147 | −6.988 | 83.752 | −4.363 | 1.00 | 14.43 |
| ATOM | 1165 | CG | TRP | A | 147 | −6.580 | 85.045 | −3.742 | 1.00 | 16.44 |
| ATOM | 1166 | CD2 | TRP | A | 147 | −6.371 | 85.292 | −2.353 | 1.00 | 15.61 |
| ATOM | 1167 | CE2 | TRP | A | 147 | −5.855 | 86.588 | −2.222 | 1.00 | 13.55 |
| ATOM | 1168 | CE3 | TRP | A | 147 | −6.556 | 84.520 | −1.195 | 1.00 | 13.91 |
| ATOM | 1169 | CD1 | TRP | A | 147 | −6.209 | 86.192 | −4.389 | 1.00 | 17.35 |
| ATOM | 1170 | NE1 | TRP | A | 147 | −5.760 | 87.122 | −3.482 | 1.00 | 16.82 |
| ATOM | 1171 | CZ2 | TRP | A | 147 | −5.526 | 87.138 | −0.999 | 1.00 | 15.71 |
| ATOM | 1172 | CZ3 | TRP | A | 147 | −6.225 | 85.066 | 0.023 | 1.00 | 13.54 |
| ATOM | 1173 | CH2 | TRP | A | 147 | −5.714 | 86.365 | 0.114 | 1.00 | 14.81 |
| ATOM | 1174 | C | TRP | A | 147 | −6.589 | 81.238 | −4.308 | 1.00 | 19.27 |
| ATOM | 1175 | O | TRP | A | 147 | −7.401 | 80.620 | −3.611 | 1.00 | 19.35 |
| ATOM | 1176 | N | ASP | A | 148 | −6.059 | 80.731 | −5.416 | 1.00 | 18.47 |
| ATOM | 1177 | CA | ASP | A | 148 | −6.416 | 79.405 | −5.904 | 1.00 | 19.12 |
| ATOM | 1178 | CB | ASP | A | 148 | −5.801 | 79.141 | −7.275 | 1.00 | 24.71 |
| ATOM | 1179 | CG | ASP | A | 148 | −6.709 | 79.536 | −8.404 | 1.00 | 26.35 |
| ATOM | 1180 | OD1 | ASP | A | 148 | −7.852 | 79.946 | −8.136 | 1.00 | 32.40 |
| ATOM | 1181 | OD2 | ASP | A | 148 | −6.284 | 79.423 | −9.569 | 1.00 | 32.06 |
| ATOM | 1182 | C | ASP | A | 148 | −5.973 | 78.328 | −4.927 | 1.00 | 18.33 |
| ATOM | 1183 | O | ASP | A | 148 | −6.797 | 77.536 | −4.486 | 1.00 | 23.56 |
| ATOM | 1184 | N | VAL | A | 149 | −4.679 | 78.276 | −4.603 | 1.00 | 18.47 |
| ATOM | 1185 | CA | VAL | A | 149 | −4.193 | 77.281 | −3.647 | 1.00 | 15.91 |
| ATOM | 1186 | CB | VAL | A | 149 | −2.678 | 77.225 | −3.513 | 1.00 | 14.54 |
| ATOM | 1187 | CG1 | VAL | A | 149 | −2.136 | 76.214 | −4.447 | 1.00 | 17.43 |
| ATOM | 1188 | CG2 | VAL | A | 149 | −2.055 | 78.590 | −3.729 | 1.00 | 16.13 |
| ATOM | 1189 | C | VAL | A | 149 | −4.757 | 77.549 | −2.271 | 1.00 | 18.11 |
| ATOM | 1190 | O | VAL | A | 149 | −4.897 | 76.633 | −1.467 | 1.00 | 21.98 |
| ATOM | 1191 | N | PHE | A | 150 | −5.032 | 78.814 | −1.979 | 1.00 | 20.18 |
| ATOM | 1192 | CA | PHE | A | 150 | −5.629 | 79.173 | −0.703 | 1.00 | 21.84 |
| ATOM | 1193 | CB | PHE | A | 150 | −5.931 | 80.666 | −0.669 | 1.00 | 19.11 |
| ATOM | 1194 | CG | PHE | A | 150 | −6.441 | 81.152 | 0.651 | 1.00 | 22.86 |
| ATOM | 1195 | CD1 | PHE | A | 150 | −5.572 | 81.689 | 1.586 | 1.00 | 22.65 |
| ATOM | 1196 | CD2 | PHE | A | 150 | −7.794 | 81.091 | 0.955 | 1.00 | 26.06 |
| ATOM | 1197 | CE1 | PHE | A | 150 | −6.045 | 82.158 | 2.800 | 1.00 | 26.82 |
| ATOM | 1198 | CE2 | PHE | A | 150 | −8.274 | 81.561 | 2.171 | 1.00 | 22.46 |
| ATOM | 1199 | CZ | PHE | A | 150 | −7.400 | 82.093 | 3.091 | 1.00 | 25.45 |
| ATOM | 1200 | C | PHE | A | 150 | −6.933 | 78.382 | −0.581 | 1.00 | 24.16 |
| ATOM | 1201 | O | PHE | A | 150 | −7.214 | 77.769 | 0.453 | 1.00 | 28.22 |
| ATOM | 1202 | N | GLY | A | 151 | −7.715 | 78.379 | −1.656 | 1.00 | 22.56 |
| ATOM | 1203 | CA | GLY | A | 151 | −8.970 | 77.659 | −1.656 | 1.00 | 20.54 |
| ATOM | 1204 | C | GLY | A | 151 | −8.761 | 76.182 | −1.417 | 1.00 | 21.70 |
| ATOM | 1205 | O | GLY | A | 151 | −9.504 | 75.565 | −0.654 | 1.00 | 25.57 |
| ATOM | 1206 | N | LEU | A | 152 | −7.745 | 75.610 | −2.049 | 1.00 | 18.26 |
| ATOM | 1207 | CA | LEU | A | 152 | −7.460 | 74.193 | −1.876 | 1.00 | 19.08 |
| ATOM | 1208 | CB | LEU | A | 152 | −6.304 | 73.770 | −2.782 | 1.00 | 19.81 |
| ATOM | 1209 | CG | LEU | A | 152 | −5.838 | 72.318 | −2.680 | 1.00 | 19.38 |
| ATOM | 1210 | CD1 | LEU | A | 152 | −6.969 | 71.381 | −3.103 | 1.00 | 16.96 |
| ATOM | 1211 | CD2 | LEU | A | 152 | −4.615 | 72.127 | −3.538 | 1.00 | 11.13 |
| ATOM | 1212 | C | LEU | A | 152 | −7.093 | 73.916 | −0.425 | 1.00 | 24.24 |
| ATOM | 1213 | O | LEU | A | 152 | −7.667 | 73.032 | 0.219 | 1.00 | 26.47 |
| ATOM | 1214 | N | SER | A | 153 | −6.141 | 74.682 | 0.095 | 1.00 | 24.56 |
| ATOM | 1215 | CA | SER | A | 153 | −5.687 | 74.517 | 1.468 | 1.00 | 22.73 |
| ATOM | 1216 | CB | SER | A | 153 | −4.591 | 75.528 | 1.770 | 1.00 | 21.22 |
| ATOM | 1217 | OG | SER | A | 153 | −3.572 | 75.448 | 0.787 | 1.00 | 21.77 |
| ATOM | 1218 | C | SER | A | 153 | −6.842 | 74.665 | 2.444 | 1.00 | 22.43 |
| ATOM | 1219 | O | SER | A | 153 | −7.041 | 73.823 | 3.316 | 1.00 | 24.75 |
| ATOM | 1220 | N | SER | A | 154 | −7.642 | 75.698 | 2.245 | 1.00 | 22.50 |
| ATOM | 1221 | CA | SER | A | 154 | −8.792 | 75.950 | 3.088 | 1.00 | 25.72 |
| ATOM | 1222 | CB | SER | A | 154 | −9.588 | 77.108 | 2.497 | 1.00 | 25.08 |
| ATOM | 1223 | OG | SER | A | 154 | −10.672 | 77.472 | 3.328 | 1.00 | 38.08 |
| ATOM | 1224 | C | SER | A | 154 | −9.662 | 74.688 | 3.218 | 1.00 | 29.51 |
| ATOM | 1225 | O | SER | A | 154 | −10.140 | 74.356 | 4.310 | 1.00 | 33.28 |
| ATOM | 1226 | N | ALA | A | 155 | −9.786 | 73.941 | 2.121 | 1.00 | 31.53 |
| ATOM | 1227 | CA | ALA | A | 155 | −10.582 | 72.713 | 2.099 | 1.00 | 29.45 |
| ATOM | 1228 | CB | ALA | A | 155 | −11.038 | 72.396 | 0.696 | 1.00 | 30.93 |
| ATOM | 1229 | C | ALA | A | 155 | −9.846 | 71.523 | 2.683 | 1.00 | 28.62 |
| ATOM | 1230 | O | ALA | A | 155 | −10.473 | 70.657 | 3.281 | 1.00 | 32.89 |
| ATOM | 1231 | N | LEU | A | 156 | −8.530 | 71.455 | 2.485 | 1.00 | 28.48 |
| ATOM | 1232 | CA | LEU | A | 156 | −7.739 | 70.355 | 3.032 | 1.00 | 23.61 |
| ATOM | 1233 | CB | LEU | A | 156 | −6.323 | 70.368 | 2.476 | 1.00 | 23.88 |
| ATOM | 1234 | CG | LEU | A | 156 | −6.043 | 69.867 | 1.061 | 1.00 | 20.69 |
| ATOM | 1235 | CD1 | LEU | A | 156 | −4.587 | 70.132 | 0.729 | 1.00 | 20.27 |
| ATOM | 1236 | CD2 | LEU | A | 156 | −6.325 | 68.394 | 0.958 | 1.00 | 20.84 |
| ATOM | 1237 | C | LEU | A | 156 | −7.688 | 70.488 | 4.547 | 1.00 | 25.37 |
| ATOM | 1238 | O | LEU | A | 156 | −7.558 | 69.490 | 5.262 | 1.00 | 26.18 |
| ATOM | 1239 | N | LEU | A | 157 | −7.773 | 71.726 | 5.036 | 1.00 | 25.81 |
| ATOM | 1240 | CA | LEU | A | 157 | −7.770 | 71.981 | 6.474 | 1.00 | 25.36 |
| ATOM | 1241 | CB | LEU | A | 157 | −7.557 | 73.466 | 6.775 | 1.00 | 19.73 |
| ATOM | 1242 | CG | LEU | A | 157 | −6.135 | 74.027 | 6.673 | 1.00 | 16.53 |
| ATOM | 1243 | CD1 | LEU | A | 157 | −6.111 | 75.418 | 7.270 | 1.00 | 17.68 |
| ATOM | 1244 | CD2 | LEU | A | 157 | −5.165 | 73.150 | 7.431 | 1.00 | 15.33 |
| ATOM | 1245 | C | LEU | A | 157 | −9.076 | 71.470 | 7.107 | 1.00 | 29.31 |
| ATOM | 1246 | O | LEU | A | 157 | −9.111 | 71.079 | 8.279 | 1.00 | 32.51 |
| ATOM | 1247 | N | LYS | A | 158 | −10.161 | 71.500 | 6.341 | 1.00 | 33.23 |
| ATOM | 1248 | CA | LYS | A | 158 | −11.442 | 70.982 | 6.814 | 1.00 | 31.84 |
| ATOM | 1249 | CB | LYS | A | 158 | −12.553 | 71.355 | 5.837 | 1.00 | 33.31 |
| ATOM | 1250 | CG | LYS | A | 158 | −12.780 | 72.845 | 5.745 | 1.00 | 34.09 |
| ATOM | 1251 | CD | LYS | A | 158 | −13.850 | 73.167 | 4.738 | 1.00 | 41.31 |
| ATOM | 1252 | CE | LYS | A | 158 | −14.186 | 74.649 | 4.754 | 1.00 | 47.04 |
| ATOM | 1253 | NZ | LYS | A | 158 | −15.362 | 74.923 | 3.886 | 1.00 | 56.43 |
| ATOM | 1254 | C | LYS | A | 158 | −11.289 | 69.460 | 6.905 | 1.00 | 30.71 |
| ATOM | 1255 | O | LYS | A | 158 | −11.770 | 68.836 | 7.848 | 1.00 | 34.49 |
| ATOM | 1256 | N | GLY | A | 159 | −10.570 | 68.884 | 5.942 | 1.00 | 30.71 |
| ATOM | 1257 | CA | GLY | A | 159 | −10.313 | 67.453 | 5.930 | 1.00 | 28.93 |
| ATOM | 1258 | C | GLY | A | 159 | −9.447 | 67.040 | 7.111 | 1.00 | 30.81 |
| ATOM | 1259 | O | GLY | A | 159 | −9.690 | 66.003 | 7.732 | 1.00 | 34.67 |
| ATOM | 1260 | N | TYR | A | 160 | −8.440 | 67.851 | 7.431 | 1.00 | 28.73 |
| ATOM | 1261 | CA | TYR | A | 160 | −7.556 | 67.575 | 8.556 | 1.00 | 29.15 |
| ATOM | 1262 | CB | TYR | A | 160 | −6.378 | 68.554 | 8.556 | 1.00 | 29.86 |
| ATOM | 1263 | CG | TYR | A | 160 | −5.181 | 68.055 | 7.780 | 1.00 | 28.35 |
| ATOM | 1264 | CD1 | TYR | A | 160 | −4.828 | 68.607 | 6.543 | 1.00 | 26.01 |
| ATOM | 1265 | CE1 | TYR | A | 160 | −3.727 | 68.124 | 5.830 | 1.00 | 25.65 |
| ATOM | 1266 | CD2 | TYR | A | 160 | −4.412 | 67.017 | 8.279 | 1.00 | 26.25 |
| ATOM | 1267 | CE2 | TYR | A | 160 | −3.321 | 66.530 | 7.584 | 1.00 | 26.20 |
| ATOM | 1268 | CZ | TYR | A | 160 | −2.977 | 67.076 | 6.365 | 1.00 | 28.06 |
| ATOM | 1269 | OH | TYR | A | 160 | −1.884 | 66.546 | 5.711 | 1.00 | 25.91 |
| ATOM | 1270 | C | TYR | A | 160 | −8.313 | 67.646 | 9.884 | 1.00 | 32.18 |
| ATOM | 1271 | O | TYR | A | 160 | −8.034 | 66.884 | 10.812 | 1.00 | 33.45 |
| ATOM | 1272 | N | ALA | A | 161 | −9.262 | 68.571 | 9.976 | 1.00 | 33.22 |
| ATOM | 1273 | CA | ALA | A | 161 | −10.074 | 68.734 | 11.180 | 1.00 | 30.35 |
| ATOM | 1274 | CB | ALA | A | 161 | −10.995 | 69.919 | 11.021 | 1.00 | 30.11 |
| ATOM | 1275 | C | ALA | A | 161 | −10.890 | 67.470 | 11.433 | 1.00 | 31.40 |
| ATOM | 1276 | O | ALA | A | 161 | −10.863 | 66.911 | 12.525 | 1.00 | 32.99 |
| ATOM | 1277 | N | LEU | A | 162 | −11.593 | 67.012 | 10.405 | 1.00 | 30.20 |
| ATOM | 1278 | CA | LEU | A | 162 | −12.405 | 65.813 | 10.501 | 1.00 | 31.99 |
| ATOM | 1279 | CB | LEU | A | 162 | −13.156 | 65.587 | 9.186 | 1.00 | 32.01 |
| ATOM | 1280 | CG | LEU | A | 162 | −14.116 | 66.719 | 8.801 | 1.00 | 33.82 |
| ATOM | 1281 | CD1 | LEU | A | 162 | −14.867 | 66.349 | 7.545 | 1.00 | 34.17 |
| ATOM | 1282 | CD2 | LEU | A | 162 | −15.096 | 66.997 | 9.933 | 1.00 | 34.51 |
| ATOM | 1283 | C | LEU | A | 162 | −11.580 | 64.580 | 10.877 | 1.00 | 32.42 |
| ATOM | 1284 | O | LEU | A | 162 | −12.002 | 63.767 | 11.696 | 1.00 | 35.19 |
| ATOM | 1285 | N | ALA | A | 163 | −10.396 | 64.453 | 10.291 | 1.00 | 34.99 |
| ATOM | 1286 | CA | ALA | A | 163 | −9.504 | 63.325 | 10.573 | 1.00 | 32.51 |
| ATOM | 1287 | CB | ALA | A | 163 | −8.289 | 63.395 | 9.670 | 1.00 | 28.78 |
| ATOM | 1288 | C | ALA | A | 163 | −9.061 | 63.273 | 12.038 | 1.00 | 30.64 |
| ATOM | 1289 | O | ALA | A | 163 | −8.745 | 62.217 | 12.571 | 1.00 | 30.60 |
| ATOM | 1290 | N | LEU | A | 164 | −8.995 | 64.428 | 12.674 | 1.00 | 29.60 |
| ATOM | 1291 | CA | LEU | A | 164 | −8.578 | 64.478 | 14.054 | 1.00 | 30.80 |
| ATOM | 1292 | CB | LEU | A | 164 | −7.639 | 65.666 | 14.274 | 1.00 | 30.81 |
| ATOM | 1293 | CG | LEU | A | 164 | −6.284 | 65.550 | 13.570 | 1.00 | 29.00 |
| ATOM | 1294 | CD1 | LEU | A | 164 | −5.583 | 66.875 | 13.576 | 1.00 | 30.09 |
| ATOM | 1295 | CD2 | LEU | A | 164 | −5.434 | 64.509 | 14.245 | 1.00 | 28.45 |
| ATOM | 1296 | C | LEU | A | 164 | −9.778 | 64.529 | 14.993 | 1.00 | 34.67 |
| ATOM | 1297 | O | LEU | A | 164 | −9.633 | 64.811 | 16.179 | 1.00 | 37.15 |
| ATOM | 1298 | N | GLY | A | 165 | −10.964 | 64.258 | 14.455 | 1.00 | 35.40 |
| ATOM | 1299 | CA | GLY | A | 165 | −12.172 | 64.258 | 15.265 | 1.00 | 34.11 |
| ATOM | 1300 | C | GLY | A | 165 | −12.637 | 65.606 | 15.781 | 1.00 | 35.01 |
| ATOM | 1301 | O | GLY | A | 165 | −13.465 | 65.680 | 16.694 | 1.00 | 38.99 |
| ATOM | 1302 | N | LYS | A | 166 | −12.111 | 66.678 | 15.208 | 1.00 | 34.42 |
| ATOM | 1303 | CA | LYS | A | 166 | −12.490 | 68.021 | 15.619 | 1.00 | 34.45 |
| ATOM | 1304 | CB | LYS | A | 166 | −11.267 | 68.924 | 15.571 | 1.00 | 31.93 |
| ATOM | 1305 | CG | LYS | A | 166 | −10.232 | 68.560 | 16.594 | 1.00 | 32.57 |
| ATOM | 1306 | CD | LYS | A | 166 | −10.711 | 68.973 | 17.956 | 1.00 | 34.75 |
| ATOM | 1307 | CE | LYS | A | 166 | −9.756 | 68.522 | 19.022 | 1.00 | 38.74 |
| ATOM | 1308 | NZ | LYS | A | 166 | −10.078 | 69.161 | 20.313 | 1.00 | 40.84 |
| ATOM | 1309 | C | LYS | A | 166 | −13.557 | 68.535 | 14.666 | 1.00 | 37.47 |
| ATOM | 1310 | O | LYS | A | 166 | −13.825 | 67.901 | 13.642 | 1.00 | 41.52 |
| ATOM | 1311 | N | GLU | A | 167 | −14.189 | 69.660 | 14.994 | 1.00 | 38.98 |
| ATOM | 1312 | CA | GLU | A | 167 | −15.202 | 70.205 | 14.094 | 1.00 | 41.09 |
| ATOM | 1313 | CB | GLU | A | 167 | −15.947 | 71.398 | 14.713 | 1.00 | 45.19 |
| ATOM | 1314 | CG | GLU | A | 167 | −15.110 | 72.638 | 15.025 | 1.00 | 55.15 |
| ATOM | 1315 | CD | GLU | A | 167 | −14.565 | 72.661 | 16.448 | 1.00 | 61.94 |
| ATOM | 1316 | OE1 | GLU | A | 167 | −14.118 | 73.742 | 16.887 | 1.00 | 62.92 |
| ATOM | 1317 | OE2 | GLU | A | 167 | −14.584 | 71.611 | 17.131 | 1.00 | 64.60 |
| ATOM | 1318 | C | GLU | A | 167 | −14.525 | 70.605 | 12.780 | 1.00 | 41.39 |
| ATOM | 1319 | O | GLU | A | 167 | −13.339 | 70.935 | 12.761 | 1.00 | 41.98 |
| ATOM | 1320 | N | GLU | A | 168 | −15.285 | 70.581 | 11.692 | 1.00 | 39.45 |
| ATOM | 1321 | CA | GLU | A | 168 | −14.779 | 70.918 | 10.365 | 1.00 | 38.77 |
| ATOM | 1322 | CB | GLU | A | 168 | −15.943 | 70.959 | 9.387 | 1.00 | 40.01 |
| ATOM | 1323 | CG | GLU | A | 168 | −15.535 | 70.844 | 7.951 | 1.00 | 42.60 |
| ATOM | 1324 | CD | GLU | A | 168 | −16.721 | 70.635 | 7.056 | 1.00 | 45.09 |
| ATOM | 1325 | OE1 | GLU | A | 168 | −17.408 | 71.626 | 6.740 | 1.00 | 51.44 |
| ATOM | 1326 | OE2 | GLU | A | 168 | −16.979 | 69.477 | 6.677 | 1.00 | 50.69 |
| ATOM | 1327 | C | GLU | A | 168 | −13.965 | 72.212 | 10.255 | 1.00 | 37.65 |
| ATOM | 1328 | O | GLU | A | 168 | −12.966 | 72.270 | 9.533 | 1.00 | 37.39 |
| ATOM | 1329 | N | ASN | A | 169 | −14.389 | 73.247 | 10.965 | 1.00 | 34.88 |
| ATOM | 1330 | CA | ASN | A | 169 | −13.696 | 74.527 | 10.925 | 1.00 | 31.36 |
| ATOM | 1331 | CB | ASN | A | 169 | −14.710 | 75.654 | 10.976 | 1.00 | 38.29 |
| ATOM | 1332 | CG | ASN | A | 169 | −15.529 | 75.726 | 9.732 | 1.00 | 46.39 |
| ATOM | 1333 | OD1 | ASN | A | 169 | −14.993 | 75.919 | 8.646 | 1.00 | 52.41 |
| ATOM | 1334 | ND2 | ASN | A | 169 | −16.833 | 75.544 | 9.865 | 1.00 | 52.70 |
| ATOM | 1335 | C | ASN | A | 169 | −12.677 | 74.717 | 12.029 | 1.00 | 29.02 |
| ATOM | 1336 | O | ASN | A | 169 | −12.264 | 75.839 | 12.318 | 1.00 | 25.13 |
| ATOM | 1337 | N | PHE | A | 170 | −12.236 | 73.618 | 12.617 | 1.00 | 29.52 |
| ATOM | 1338 | CA | PHE | A | 170 | −11.276 | 73.687 | 13.702 | 1.00 | 30.89 |
| ATOM | 1339 | CB | PHE | A | 170 | −10.938 | 72.275 | 14.191 | 1.00 | 34.08 |
| ATOM | 1340 | CG | PHE | A | 170 | −10.030 | 72.248 | 15.377 | 1.00 | 38.01 |
| ATOM | 1341 | CD1 | PHE | A | 170 | −10.418 | 72.827 | 16.575 | 1.00 | 41.53 |
| ATOM | 1342 | CD2 | PHE | A | 170 | −8.778 | 71.658 | 15.293 | 1.00 | 39.11 |
| ATOM | 1343 | CE1 | PHE | A | 170 | −9.571 | 72.824 | 17.675 | 1.00 | 40.36 |
| ATOM | 1344 | CE2 | PHE | A | 170 | −7.925 | 71.649 | 16.385 | 1.00 | 41.21 |
| ATOM | 1345 | CZ | PHE | A | 170 | −8.326 | 72.235 | 17.580 | 1.00 | 42.47 |
| ATOM | 1346 | C | PHE | A | 170 | −10.012 | 74.464 | 13.305 | 1.00 | 32.24 |
| ATOM | 1347 | O | PHE | A | 170 | −9.496 | 75.255 | 14.102 | 1.00 | 32.82 |
| ATOM | 1348 | N | PHE | A | 171 | −9.537 | 74.269 | 12.072 | 1.00 | 30.18 |
| ATOM | 1349 | CA | PHE | A | 171 | −8.338 | 74.959 | 11.595 | 1.00 | 25.80 |
| ATOM | 1350 | CB | PHE | A | 171 | −7.436 | 74.018 | 10.780 | 1.00 | 21.23 |
| ATOM | 1351 | CG | PHE | A | 171 | −6.801 | 72.922 | 11.584 | 1.00 | 17.74 |
| ATOM | 1352 | CD1 | PHE | A | 171 | −6.984 | 71.592 | 11.232 | 1.00 | 19.14 |
| ATOM | 1353 | CD2 | PHE | A | 171 | −6.028 | 73.212 | 12.699 | 1.00 | 20.58 |
| ATOM | 1354 | CE1 | PHE | A | 171 | −6.409 | 70.559 | 11.986 | 1.00 | 19.44 |
| ATOM | 1355 | CE2 | PHE | A | 171 | −5.449 | 72.188 | 13.457 | 1.00 | 19.74 |
| ATOM | 1356 | CZ | PHE | A | 171 | −5.644 | 70.861 | 13.095 | 1.00 | 18.77 |
| ATOM | 1357 | C | PHE | A | 171 | −8.720 | 76.142 | 10.722 | 1.00 | 27.98 |
| ATOM | 1358 | O | PHE | A | 171 | −8.301 | 77.282 | 10.968 | 1.00 | 26.96 |
| ATOM | 1359 | N | ALA | A | 172 | −9.573 | 75.874 | 9.737 | 1.00 | 27.10 |
| ATOM | 1360 | Q | ALA | A | 172 | −10.009 | 76.880 | 8.770 | 1.00 | 23.60 |
| ATOM | 1361 | CB | ALA | A | 172 | −10.996 | 76.276 | 7.798 | 1.00 | 20.83 |
| ATOM | 1362 | C | ALA | A | 172 | −10.542 | 78.191 | 9.310 | 1.00 | 25.20 |
| ATOM | 1363 | O | ALA | A | 172 | −10.477 | 79.204 | 8.623 | 1.00 | 27.68 |
| ATOM | 1364 | N | ARG | A | 173 | −11.044 | 78.195 | 10.540 | 1.00 | 27.56 |
| ATOM | 1365 | CA | MG | A | 173 | −11.573 | 79.429 | 11.098 | 1.00 | 28.04 |
| ATOM | 1366 | CB | ARG | A | 173 | −12.374 | 79.170 | 12.377 | 1.00 | 29.47 |
| ATOM | 1367 | CG | ARG | A | 173 | −11.559 | 79.001 | 13.633 | 1.00 | 35.23 |
| ATOM | 1368 | CD | MG | A | 173 | −12.452 | 78.858 | 14.868 | 1.00 | 40.18 |
| ATOM | 1369 | NE | MG | A | 173 | −12.898 | 77.482 | 15.106 | 1.00 | 44.83 |
| ATOM | 1370 | CZ | ARG | A | 173 | −14.162 | 77.074 | 15.017 | 1.00 | 48.14 |
| ATOM | 1371 | NH1 | MG | A | 173 | −15.122 | 77.934 | 14.695 | 1.00 | 48.64 |
| ATOM | 1372 | NH2 | MG | A | 173 | −14.468 | 75.800 | 15.240 | 1.00 | 44.20 |
| ATOM | 1373 | C | MG | A | 173 | −10.433 | 80.401 | 11.355 | 1.00 | 30.23 |
| ATOM | 1374 | O | ARG | A | 173 | −10.657 | 81.591 | 11.584 | 1.00 | 32.24 |
| ATOM | 1375 | N | HIS | A | 174 | −9.206 | 79.889 | 11.314 | 1.00 | 28.30 |
| ATOM | 1376 | CA | HIS | A | 174 | −8.023 | 80.713 | 11.537 | 1.00 | 28.03 |
| ATOM | 1377 | CB | HIS | A | 174 | −7.051 | 79.999 | 12.473 | 1.00 | 25.87 |
| ATOM | 1378 | CG | HIS | A | 174 | −7.622 | 79.688 | 13.816 | 1.00 | 26.33 |
| ATOM | 1379 | CD2 | HIS | A | 174 | −8.121 | 78.536 | 14.326 | 1.00 | 28.81 |
| ATOM | 1380 | ND1 | HIS | A | 174 | −7.705 | 80.623 | 14.826 | 1.00 | 27.96 |
| ATOM | 1381 | CE1 | HIS | A | 174 | −8.228 | 80.059 | 15.900 | 1.00 | 28.08 |
| ATOM | 1382 | NE2 | HIS | A | 174 | −8.488 | 78.793 | 15.624 | 1.00 | 27.09 |
| ATOM | 1383 | C | HIS | A | 174 | −7.305 | 81.064 | 10.235 | 1.00 | 27.63 |
| ATOM | 1384 | O | HIS | A | 174 | −6.371 | 81.865 | 10.240 | 1.00 | 31.26 |
| ATOM | 1385 | N | PHE | A | 175 | −7.734 | 80.442 | 9.138 | 1.00 | 26.90 |
| ATOM | 1386 | CA | PHE | A | 175 | −7.165 | 80.631 | 7.801 | 1.00 | 22.15 |
| ATOM | 1387 | CB | PHE | A | 175 | −7.222 | 79.289 | 7.074 | 1.00 | 21.26 |
| ATOM | 1388 | CG | PHE | A | 175 | −6.293 | 79.176 | 5.911 | 1.00 | 23.40 |
| ATOM | 1389 | CD1 | PHE | A | 175 | −6.652 | 78.420 | 4.803 | 1.00 | 20.93 |
| ATOM | 1390 | CD2 | PHE | A | 175 | −5.046 | 79.781 | 5.931 | 1.00 | 19.53 |
| ATOM | 1391 | CE1 | PHE | A | 175 | −5.789 | 78.266 | 3.741 | 1.00 | 19.79 |
| ATOM | 1392 | CE2 | PHE | A | 175 | −4.170 | 79.630 | 4.866 | 1.00 | 19.63 |
| ATOM | 1393 | CZ | PHE | A | 175 | −4.545 | 78.870 | 3.770 | 1.00 | 19.09 |
| ATOM | 1394 | C | PHE | A | 175 | −8.030 | 81.655 | 7.071 | 1.00 | 23.58 |
| ATOM | 1395 | O | PHE | A | 175 | −9.032 | 81.299 | 6.443 | 1.00 | 25.37 |
| ATOM | 1396 | N | LYS | A | 176 | −7.636 | 82.923 | 7.136 | 1.00 | 22.96 |
| ATOM | 1397 | CA | LYS | A | 176 | −8.412 | 84.004 | 6.529 | 1.00 | 26.79 |
| ATOM | 1398 | CB | LYS | A | 176 | −8.991 | 84.922 | 7.619 | 1.00 | 32.23 |
| ATOM | 1399 | CG | LYS | A | 176 | −9.711 | 84.185 | 8.752 | 1.00 | 37.22 |
| ATOM | 1400 | CD | LYS | A | 176 | −10.312 | 85.139 | 9.782 | 1.00 | 45.14 |
| ATOM | 1401 | CE | LYS | A | 176 | −9.271 | 86.065 | 10.408 | 1.00 | 51.76 |
| ATOM | 1402 | NZ | LYS | A | 176 | −8.282 | 85.352 | 11.277 | 1.00 | 54.44 |
| ATOM | 1403 | C | LYS | A | 176 | −7.624 | 84.842 | 5.529 | 1.00 | 26.95 |
| ATOM | 1404 | O | LYS | A | 176 | −6.447 | 85.150 | 5.736 | 1.00 | 29.87 |
| ATOM | 1405 | N | PRO | A | 177 | −8.308 | 85.327 | 4.485 | 1.00 | 24.40 |
| ATOM | 1406 | CD | PRO | A | 177 | −9.714 | 85.067 | 4.146 | 1.00 | 20.13 |
| ATOM | 1407 | CA | PRO | A | 177 | −7.685 | 86.133 | 3.445 | 1.00 | 19.31 |
| ATOM | 1408 | CB | PRO | A | 177 | −8.838 | 86.376 | 2.481 | 1.00 | 16.22 |
| ATOM | 1409 | CG | PRO | A | 177 | −9.687 | 85.187 | 2.664 | 1.00 | 13.22 |
| ATOM | 1410 | C | PRO | A | 177 | −7.084 | 87.434 | 3.903 | 1.00 | 21.14 |
| ATOM | 1411 | O | PRO | A | 177 | −6.096 | 87.883 | 3.349 | 1.00 | 25.46 |
| ATOM | 1412 | N | ASP | A | 178 | −7.660 | 88.049 | 4.917 | 1.00 | 26.69 |
| ATOM | 1413 | CA | ASP | A | 178 | −7.145 | 89.341 | 5.355 | 1.00 | 30.90 |
| ATOM | 1414 | CB | ASP | A | 178 | −8.220 | 90.162 | 6.073 | 1.00 | 40.73 |
| ATOM | 1415 | CG | ASP | A | 178 | −8.927 | 89.377 | 7.153 | 1.00 | 58.65 |
| ATOM | 1416 | OD1 | ASP | A | 178 | −9.660 | 88.411 | 6.813 | 1.00 | 67.55 |
| ATOM | 1417 | OD2 | ASP | A | 178 | −8.754 | 89.731 | 8.344 | 1.00 | 69.29 |
| ATOM | 1418 | C | ASP | A | 178 | −5.891 | 89.303 | 6.183 | 1.00 | 25.93 |
| ATOM | 1419 | O | ASP | A | 178 | −5.154 | 90.282 | 6.225 | 1.00 | 27.92 |
| ATOM | 1420 | N | ASP | A | 179 | −5.620 | 88.180 | 6.828 | 1.00 | 20.77 |
| ATOM | 1421 | CA | ASP | A | 179 | −4.432 | 88.129 | 7.645 | 1.00 | 20.37 |
| ATOM | 1422 | CB | ASP | A | 179 | −4.790 | 88.332 | 9.120 | 1.00 | 24.28 |
| ATOM | 1423 | CG | ASP | A | 179 | −5.553 | 87.157 | 9.717 | 1.00 | 28.49 |
| ATOM | 1424 | OD1 | ASP | A | 179 | −5.957 | 86.249 | 8.967 | 1.00 | 32.76 |
| ATOM | 1425 | OD2 | ASP | A | 179 | −5.750 | 87.134 | 10.953 | 1.00 | 34.31 |
| ATOM | 1426 | C | ASP | A | 179 | −3.550 | 86.912 | 7.499 | 1.00 | 20.24 |
| ATOM | 1427 | O | ASP | A | 179 | −2.568 | 86.807 | 8.224 | 1.00 | 22.73 |
| ATOM | 1428 | N | THR | A | 180 | −3.870 | 85.996 | 6.587 | 1.00 | 18.73 |
| ATOM | 1429 | CA | THR | A | 180 | −3.035 | 84.809 | 6.453 | 1.00 | 19.30 |
| ATOM | 1430 | CB | THR | A | 180 | −3.533 | 83.818 | 5.372 | 1.00 | 20.49 |
| ATOM | 1431 | OG1 | THR | A | 180 | −2.592 | 82.741 | 5.254 | 1.00 | 20.62 |
| ATOM | 1432 | CG2 | THR | A | 180 | −3.657 | 84.488 | 4.024 | 1.00 | 18.20 |
| ATOM | 1433 | C | THR | A | 180 | −1.577 | 85.144 | 6.173 | 1.00 | 22.74 |
| ATOM | 1434 | O | THR | A | 180 | −1.269 | 86.060 | 5.407 | 1.00 | 23.45 |
| ATOM | 1435 | N | LEU | A | 181 | −0.689 | 84.402 | 6.826 | 1.00 | 22.30 |
| ATOM | 1436 | CA | LEU | A | 181 | 0.752 | 84.558 | 6.661 | 1.00 | 19.63 |
| ATOM | 1437 | CB | LEU | A | 181 | 1.450 | 84.460 | 8.023 | 1.00 | 11.22 |
| ATOM | 1438 | CG | LEU | A | 181 | 1.280 | 85.655 | 8.947 | 1.00 | 8.88 |
| ATOM | 1439 | CD1 | LEU | A | 181 | 1.760 | 85.318 | 10.332 | 1.00 | 7.59 |
| ATOM | 1440 | CD2 | LEU | A | 181 | 2.041 | 86.823 | 8.395 | 1.00 | 9.34 |
| ATOM | 1441 | C | LEU | A | 181 | 1.315 | 83.488 | 5.702 | 1.00 | 19.45 |
| ATOM | 1442 | O | LEU | A | 181 | 2.524 | 83.287 | 5.629 | 1.00 | 22.17 |
| ATOM | 1443 | N | ALA | A | 182 | 0.441 | 82.775 | 5.003 | 1.00 | 15.84 |
| ATOM | 1444 | CA | ALA | A | 182 | 0.886 | 81.749 | 4.072 | 1.00 | 13.71 |
| ATOM | 1445 | CB | ALA | A | 182 | −0.306 | 81.111 | 3.410 | 1.00 | 15.01 |
| ATOM | 1446 | C | ALA | A | 182 | 1.797 | 82.379 | 3.017 | 1.00 | 16.85 |
| ATOM | 1447 | O | ALA | A | 182 | 1.690 | 83.571 | 2.723 | 1.00 | 15.28 |
| ATOM | 1448 | N | SER | A | 183 | 2.679 | 81.583 | 2.429 | 1.00 | 15.86 |
| ATOM | 1449 | CA | SER | A | 183 | 3.584 | 82.108 | 1.416 | 1.00 | 14.12 |
| ATOM | 1450 | CB | SER | A | 183 | 4.947 | 82.430 | 2.039 | 1.00 | 12.59 |
| ATOM | 1451 | OG | SER | A | 183 | 5.585 | 81.250 | 2.497 | 1.00 | 13.59 |
| ATOM | 1452 | C | SER | A | 183 | 3.759 | 81.130 | 0.257 | 1.00 | 13.98 |
| ATOM | 1453 | O | SER | A | 183 | 3.602 | 79.923 | 0.422 | 1.00 | 17.17 |
| ATOM | 1454 | N | VAL | A | 184 | 4.008 | 81.674 | −0.931 | 1.00 | 15.35 |
| ATOM | 1455 | CA | VAL | A | 184 | 4.241 | 80.885 | −2.133 | 1.00 | 13.86 |
| ATOM | 1456 | CB | VAL | A | 184 | 3.366 | 81.361 | −3.335 | 1.00 | 13.35 |
| ATOM | 1457 | CG1 | VAL | A | 184 | 3.625 | 80.500 | −4.554 | 1.00 | 11.12 |
| ATOM | 1458 | CG2 | VAL | A | 184 | 1.907 | 81.283 | −2.994 | 1.00 | 14.34 |
| ATOM | 1459 | C | VAL | A | 184 | 5.698 | 81.161 | −2.479 | 1.00 | 13.94 |
| ATOM | 1460 | O | VAL | A | 184 | 6.123 | 82.314 | −2.459 | 1.00 | 15.07 |
| ATOM | 1461 | N | VAL | A | 185 | 6.469 | 80.107 | −2.722 | 1.00 | 16.12 |
| ATOM | 1462 | CA | VAL | A | 185 | 7.871 | 80.237 | −3.095 | 1.00 | 18.01 |
| ATOM | 1463 | CB | VAL | A | 185 | 8.818 | 79.516 | −2.096 | 1.00 | 19.45 |
| ATOM | 1464 | CG1 | VAL | A | 185 | 10.262 | 79.683 | −2.528 | 1.00 | 14.07 |
| ATOM | 1465 | CG2 | VAL | A | 185 | 8.629 | 80.054 | −0.708 | 1.00 | 17.49 |
| ATOM | 1466 | C | VAL | A | 185 | 8.039 | 79.551 | −4.438 | 1.00 | 18.98 |
| ATOM | 1467 | O | VAL | A | 185 | 7.660 | 78.391 | −4.585 | 1.00 | 20.19 |
| ATOM | 1468 | N | LEU | A | 186 | 8.541 | 80.282 | −5.428 | 1.00 | 22.23 |
| ATOM | 1469 | CA | LEU | A | 186 | 8.781 | 79.717 | −6.760 | 1.00 | 22.67 |
| ATOM | 1470 | CB | LEU | A | 186 | 8.468 | 80.748 | −7.861 | 1.00 | 17.46 |
| ATOM | 1471 | CG | LEU | A | 186 | 7.117 | 81.480 | −7.844 | 1.00 | 17.76 |
| ATOM | 1472 | CD1 | LEU | A | 186 | 6.993 | 82.315 | −9.092 | 1.00 | 19.25 |
| ATOM | 1473 | CD2 | LEU | A | 186 | 5.970 | 80.514 | −7.761 | 1.00 | 12.57 |
| ATOM | 1474 | C | LEU | A | 186 | 10.261 | 79.325 | −6.800 | 1.00 | 21.43 |
| ATOM | 1475 | O | LEU | A | 186 | 11.124 | 80.169 | −7.058 | 1.00 | 21.60 |
| ATOM | 1476 | N | ILE | A | 187 | 10.555 | 78.065 | −6.500 | 1.00 | 18.26 |
| ATOM | 1477 | CA | ILE | A | 187 | 11.929 | 77.586 | −6.477 | 1.00 | 18.51 |
| ATOM | 1478 | CB | ILE | A | 187 | 12.068 | 76.373 | −5.547 | 1.00 | 17.33 |
| ATOM | 1479 | CG2 | ILE | A | 187 | 13.524 | 75.915 | −5.484 | 1.00 | 17.55 |
| ATOM | 1480 | CG1 | ILE | A | 187 | 11.560 | 76.727 | −4.152 | 1.00 | 14.78 |
| ATOM | 1481 | CD1 | ILE | A | 187 | 11.608 | 75.582 | −3.201 | 1.00 | 12.56 |
| ATOM | 1482 | C | ILE | A | 187 | 12.421 | 77.183 | −7.858 | 1.00 | 24.55 |
| ATOM | 1483 | O | ILE | A | 187 | 11.688 | 76.567 | −8.632 | 1.00 | 28.10 |
| ATOM | 1484 | N | ARG | A | 188 | 13.671 | 77.509 | −8.158 | 1.00 | 27.00 |
| ATOM | 1485 | CA | ARG | A | 188 | 14.258 | 77.160 | −9.447 | 1.00 | 27.28 |
| ATOM | 1486 | CB | ARG | A | 188 | 14.659 | 78.405 | −10.241 | 1.00 | 25.39 |
| ATOM | 1487 | CG | ARG | A | 188 | 15.481 | 78.037 | −11.466 | 1.00 | 26.11 |
| ATOM | 1488 | CD | ARG | A | 188 | 16.122 | 79.207 | −12.169 | 1.00 | 26.92 |
| ATOM | 1489 | NE | ARG | A | 188 | 16.656 | 78.756 | −13.448 | 1.00 | 33.43 |
| ATOM | 1490 | CZ | ARG | A | 188 | 17.555 | 79.406 | −14.176 | 1.00 | 37.61 |
| ATOM | 1491 | NH1 | M G | A | 188 | 18.054 | 80.567 | −13.779 | 1.00 | 40.25 |
| ATOM | 1492 | NH2 | ARG | A | 188 | 17.945 | 78.890 | −15.327 | 1.00 | 45.39 |
| ATOM | 1493 | C | ARG | A | 188 | 15.494 | 76.291 | −9.302 | 1.00 | 27.56 |
| ATOM | 1494 | O | ARG | A | 188 | 16.462 | 76.678 | −8.644 | 1.00 | 28.16 |
| ATOM | 1495 | N | TYR | A | 189 | 15.463 | 75.120 | −9.921 | 1.00 | 28.98 |
| ATOM | 1496 | Q | TYR | A | 189 | 16.605 | 74.221 | −9.901 | 1.00 | 30.35 |
| ATOM | 1497 | CB | TYR | A | 189 | 16.166 | 72.799 | −9.600 | 1.00 | 28.57 |
| ATOM | 1498 | CG | TYR | A | 189 | 15.715 | 72.610 | −8.179 | 1.00 | 27.58 |
| ATOM | 1499 | CD1 | TYR | A | 189 | 14.363 | 72.586 | −7.862 | 1.00 | 30.42 |
| ATOM | 1500 | CE1 | TYR | A | 189 | 13.933 | 72.371 | −6.555 | 1.00 | 32.46 |
| ATOM | 1501 | CD2 | TYR | A | 189 | 16.639 | 72.419 | −7.154 | 1.00 | 25.30 |
| ATOM | 1502 | CE2 | TYR | A | 189 | 16.224 | 72.206 | −5.846 | 1.00 | 27.43 |
| ATOM | 1503 | CZ | TYR | A | 189 | 14.866 | 72.182 | −5.553 | 1.00 | 31.28 |
| ATOM | 1504 | OH | TYR | A | 189 | 14.434 | 71.980 | −4.262 | 1.00 | 37.45 |
| ATOM | 1505 | C | TYR | A | 189 | 17.225 | 74.335 | −11.293 | 1.00 | 30.75 |
| ATOM | 1506 | O | TYR | A | 189 | 16.643 | 73.905 | −12.278 | 1.00 | 31.49 |
| ATOM | 1507 | N | PRO | A | 190 | 18.396 | 74.974 | −11.388 | 1.00 | 29.84 |
| ATOM | 1508 | CD | PRO | A | 190 | 19.157 | 75.502 | −10.238 | 1.00 | 29.04 |
| ATOM | 1509 | CA | PRO | A | 190 | 19.131 | 75.192 | −12.631 | 1.00 | 29.41 |
| ATOM | 1510 | CB | PRO | A | 190 | 20.122 | 76.271 | −12.222 | 1.00 | 30.04 |
| ATOM | 1511 | CG | PRO | A | 190 | 20.508 | 75.816 | −10.845 | 1.00 | 27.63 |
| ATOM | 1512 | C | PRO | A | 190 | 19.899 | 74.005 | −13.163 | 1.00 | 29.09 |
| ATOM | 1513 | O | PRO | A | 190 | 20.168 | 73.057 | −12.428 | 1.00 | 31.04 |
| ATOM | 1514 | N | TYR | A | 191 | 20.220 | 74.051 | −14.454 | 1.00 | 27.02 |
| ATOM | 1515 | CA | TYR | A | 191 | 21.065 | 73.025 | −15.054 | 1.00 | 25.82 |
| ATOM | 1516 | CB | TYR | A | 191 | 20.612 | 72.611 | −16.449 | 1.00 | 24.40 |
| ATOM | 1517 | CG | TYR | A | 191 | 21.677 | 71.806 | −17.164 | 1.00 | 23.94 |
| ATOM | 1518 | CD1 | TYR | A | 191 | 21.959 | 0.497 | −16.779 | 1.00 | 24.85 |
| ATOM | 1519 | CE1 | TYR | A | 191 | 22.997 | 69.779 | −17.370 | 1.00 | 26.57 |
| ATOM | 1520 | CD2 | TYR | A | 191 | 22.463 | 72.381 | −18.169 | 1.00 | 24.25 |
| ATOM | 1521 | CE2 | TYR | A | 191 | 23.509 | 71.674 | −18.764 | 1.00 | 22.72 |
| ATOM | 1522 | CZ | TYR | A | 191 | 23.769 | 70.372 | −18.359 | 1.00 | 27.36 |
| ATOM | 1523 | OH | TYR | A | 191 | 24.791 | 69.655 | −18.938 | 1.00 | 32.34 |
| ATOM | 1524 | C | TYR | A | 191 | 22.406 | 73.737 | −15.167 | 1.00 | 26.75 |
| ATOM | 1525 | O | TYR | A | 191 | 22.477 | 74.848 | −15.697 | 1.00 | 26.19 |
| ATOM | 1526 | N | LEU | A | 192 | 23.453 | 73.131 | −14.620 | 1.00 | 27.75 |
| ATOM | 1527 | CA | LEU | A | 192 | 24.789 | 73.714 | −14.662 | 1.00 | 30.44 |
| ATOM | 1528 | CB | LEU | A | 192 | 25.134 | 74.378 | −13.322 | 1.00 | 27.32 |
| ATOM | 1529 | CG | LEU | A | 192 | 24.221 | 75.486 | −12.789 | 1.00 | 24.54 |
| ATOM | 1530 | CD1 | LEU | A | 192 | 24.644 | 75.909 | −11.386 | 1.00 | 20.65 |
| ATOM | 1531 | CD2 | LEU | A | 192 | 24.252 | 76.664 | −13.738 | 1.00 | 21.14 |
| ATOM | 1532 | C | LEU | A | 192 | 25.856 | 72.673 | −15.013 | 1.00 | 34.74 |
| ATOM | 1533 | O | LEU | A | 192 | 25.803 | 71.511 | −14.586 | 1.00 | 33.09 |
| ATOM | 1534 | N | ASP | A | 193 | 26.778 | 73.085 | −15.867 | 1.00 | 36.86 |
| ATOM | 1535 | CA | ASP | A | 193 | 27.877 | 72.250 | −16.289 | 1.00 | 42.70 |
| ATOM | 1536 | CB | ASP | A | 193 | 27.564 | 71.544 | −17.600 | 1.00 | 48.25 |
| ATOM | 1537 | CG | ASP | A | 193 | 28.732 | 70.728 | −18.106 | 1.00 | 52.17 |
| ATOM | 1538 | OD1 | ASP | A | 193 | 29.125 | 70.928 | −19.271 | 1.00 | 58.38 |
| ATOM | 1539 | OD2 | ASP | A | 193 | 29.266 | 69.897 | −17.338 | 1.00 | 53.48 |
| ATOM | 1540 | C | ASP | A | 193 | 29.032 | 73.196 | −16.494 | 1.00 | 42.38 |
| ATOM | 1541 | O | ASP | A | 193 | 29.061 | 73.951 | −17.461 | 1.00 | 46.89 |
| ATOM | 1542 | N | PRO | A | 194 | 29.994 | 73.178 | −15.574 | 1.00 | 40.26 |
| ATOM | 1543 | CD | PRO | A | 194 | 31.206 | 74.016 | −15.565 | 1.00 | 40.20 |
| ATOM | 1544 | CA | PRO | A | 194 | 29.956 | 72.294 | −14.414 | 1.00 | 38.87 |
| ATOM | 1545 | CB | PRO | A | 194 | 31.419 | 72.224 | −14.018 | 1.00 | 39.32 |
| ATOM | 1546 | CG | PRO | A | 194 | 31.863 | 73.644 | −14.250 | 1.00 | 39.54 |
| ATOM | 1547 | C | PRO | A | 194 | 29.082 | 72.863 | −13.293 | 1.00 | 40.57 |
| ATOM | 1548 | O | PRO | A | 194 | 28.773 | 74.065 | −13.261 | 1.00 | 39.81 |
| ATOM | 1549 | N | TYR | A | 195 | 28.667 | 71.976 | −12.395 | 1.00 | 38.88 |
| ATOM | 1550 | CA | TYR | A | 195 | 27.826 | 72.341 | −11.269 | 1.00 | 35.95 |
| ATOM | 1551 | CB | TYR | A | 195 | 26.869 | 71.187 | −10.933 | 1.00 | 33.96 |
| ATOM | 1552 | CG | TYR | A | 195 | 25.572 | 71.600 | −10.255 | 1.00 | 33.27 |
| ATOM | 1553 | CD1 | TYR | A | 195 | 24.390 | 71.688 | −10.984 | 1.00 | 32.32 |
| ATOM | 1554 | CE1 | TYR | A | 195 | 23.200 | 72.048 | −10.381 | 1.00 | 31.47 |
| ATOM | 1555 | CD2 | TYR | A | 195 | 25.522 | 71.887 | −8.889 | 1.00 | 29.66 |
| ATOM | 1556 | CE2 | TYR | A | 195 | 24.330 | 72.249 | −8.276 | 1.00 | 27.38 |
| ATOM | 1557 | CZ | TYR | A | 195 | 23.178 | 72.325 | −9.030 | 1.00 | 27.54 |
| ATOM | 1558 | OH | TYR | A | 195 | 21.996 | 72.665 | −8.436 | 1.00 | 25.96 |
| ATOM | 1559 | C | TYR | A | 195 | 28.726 | 72.624 | −10.072 | 1.00 | 35.66 |
| ATOM | 1560 | O | TYR | A | 195 | 29.616 | 71.832 | −9.748 | 1.00 | 34.39 |
| ATOM | 1561 | N | PRO | A | 196 | 28.505 | 73.762 | −9.402 | 1.00 | 34.12 |
| ATOM | 1562 | CD | PRO | A | 196 | 27.458 | 74.742 | −9.730 | 1.00 | 32.84 |
| ATOM | 1563 | CA | PRO | A | 196 | 29.270 | 74.183 | −8.231 | 1.00 | 34.92 |
| ATOM | 1564 | CB | PRO | A | 196 | 28.472 | 75.377 | −7.735 | 1.00 | 35.33 |
| ATOM | 1565 | CG | PRO | A | 196 | 27.915 | 75.951 | −8.995 | 1.00 | 36.48 |
| ATOM | 1566 | C | PRO | A | 196 | 29.276 | 73.076 | −7.188 | 1.00 | 40.87 |
| ATOM | 1567 | O | PRO | A | 196 | 28.257 | 72.808 | −6.558 | 1.00 | 44.01 |
| ATOM | 1568 | N | GLU | A | 197 | 30.418 | 72.425 | −7.013 | 1.00 | 46.54 |
| ATOM | 1569 | CA | GLU | A | 197 | 30.535 | 71.337 | −6.048 | 1.00 | 49.40 |
| ATOM | 1570 | CB | GLU | A | 197 | 31.992 | 70.896 | −5.916 | 1.00 | 59.62 |
| ATOM | 1571 | CG | GLU | A | 197 | 32.595 | 70.350 | −7.211 | 1.00 | 74.74 |
| ATOM | 1572 | CD | GLU | A | 197 | 34.093 | 70.061 | −7.108 | 1.00 | 82.34 |
| ATOM | 1573 | OE1 | GLU | A | 197 | 34.807 | 70.797 | −6.383 | 1.00 | 86.57 |
| ATOM | 1574 | OE2 | GLU | A | 197 | 34.558 | 69.100 | −7.765 | 1.00 | 87.43 |
| ATOM | 1575 | C | GLU | A | 197 | 30.007 | 71.757 | −4.692 | 1.00 | 45.50 |
| ATOM | 1576 | O | GLU | A | 197 | 29.395 | 70.972 | −3.985 | 1.00 | 46.60 |
| ATOM | 1577 | N | ALA | A | 198 | 30.216 | 73.018 | −4.352 | 1.00 | 43.56 |
| ATOM | 1578 | CA | ALA | A | 198 | 29.765 | 73.538 | −3.072 | 1.00 | 42.05 |
| ATOM | 1579 | CB | ALA | A | 198 | 30.214 | 74.968 | −2.908 | 1.00 | 42.58 |
| ATOM | 1580 | C | ALA | A | 198 | 28.264 | 73.443 | −2.861 | 1.00 | 41.83 |
| ATOM | 1581 | O | ALA | A | 198 | 27.805 | 73.315 | −1.728 | 1.00 | 46.61 |
| ATOM | 1582 | N | ALA | A | 199 | 27.501 | 73.501 | −3.946 | 1.00 | 36.04 |
| ATOM | 1583 | CA | ALA | A | 199 | 26.052 | 73.450 | −3.852 | 1.00 | 32.51 |
| ATOM | 1584 | CB | ALA | A | 199 | 25.430 | 74.173 | −5.019 | 1.00 | 33.11 |
| ATOM | 1585 | C | ALA | A | 199 | 25.512 | 72.044 | −3.772 | 1.00 | 33.23 |
| ATOM | 1586 | O | ALA | A | 199 | 24.307 | 71.837 | −3.900 | 1.00 | 38.62 |
| ATOM | 1587 | N | ILE | A | 200 | 26.397 | 71.075 | −3.590 | 1.00 | 32.94 |
| ATOM | 1588 | CA | ILE | A | 200 | 25.973 | 69.687 | −3.508 | 1.00 | 34.13 |
| ATOM | 1589 | CB | ILE | A | 200 | 26.565 | 68.846 | −4.644 | 1.00 | 30.62 |
| ATOM | 1590 | CG2 | ILE | A | 200 | 26.086 | 67.409 | −4.527 | 1.00 | 19.56 |
| ATOM | 1591 | CG1 | ILE | A | 200 | 26.154 | 69.451 | −5.988 | 1.00 | 31.15 |
| ATOM | 1592 | CD1 | ILE | A | 200 | 27.065 | 69.109 | −7.124 | 1.00 | 34.14 |
| ATOM | 1593 | C | ILE | A | 200 | 26.353 | 69.073 | −2.182 | 1.00 | 37.98 |
| ATOM | 1594 | O | ILE | A | 200 | 27.525 | 68.817 | −1.909 | 1.00 | 42.30 |
| ATOM | 1595 | N | LYS | A | 201 | 25.343 | 68.844 | −1.356 | 1.00 | 41.21 |
| ATOM | 1596 | CA | LYS | A | 201 | 25.541 | 68.258 | −0.045 | 1.00 | 42.08 |
| ATOM | 1597 | CB | LYS | A | 201 | 24.485 | 68.776 | 0.935 | 1.00 | 44.17 |
| ATOM | 1598 | CG | LYS | A | 201 | 24.592 | 70.275 | 1.203 | 1.00 | 47.76 |
| ATOM | 1599 | CD | LYS | A | 201 | 23.219 | 70.918 | 1.218 | 1.00 | 52.10 |
| ATOM | 1600 | CE | LYS | A | 201 | 23.302 | 72.423 | 1.021 | 1.00 | 52.22 |
| ATOM | 1601 | NZ | LYS | A | 201 | 21.940 | 73.026 | 0.955 | 1.00 | 52.59 |
| ATOM | 1602 | C | LYS | A | 201 | 25.443 | 66.761 | −0.179 | 1.00 | 39.59 |
| ATOM | 1603 | O | LYS | A | 201 | 24.587 | 66.255 | −0.886 | 1.00 | 42.27 |
| ATOM | 1604 | N | THR | A | 202 | 26.338 | 66.052 | 0.485 | 1.00 | 40.47 |
| ATOM | 1605 | CA | THR | A | 202 | 26.329 | 64.605 | 0.424 | 1.00 | 42.05 |
| ATOM | 1606 | CB | THR | A | 202 | 27.682 | 64.062 | −0.114 | 1.00 | 38.96 |
| ATOM | 1607 | OG1 | THR | A | 202 | 27.900 | 62.727 | 0.356 | 1.00 | 42.95 |
| ATOM | 1608 | CG2 | THR | A | 202 | 28.838 | 64.970 | 0.276 | 1.00 | 40.84 |
| ATOM | 1609 | C | THR | A | 202 | 25.908 | 64.022 | 1.781 | 1.00 | 42.98 |
| ATOM | 1610 | O | THR | A | 202 | 26.553 | 64.247 | 2.809 | 1.00 | 47.33 |
| ATOM | 1611 | N | ALA | A | 203 | 24.750 | 63.369 | 1.785 | 1.00 | 41.07 |
| ATOM | 1612 | CA | ALA | A | 203 | 24.195 | 62.776 | 2.995 | 1.00 | 42.49 |
| ATOM | 1613 | Q | ALA | A | 203 | 22.824 | 62.179 | 2.713 | 1.00 | 36.69 |
| ATOM | 1614 | C | ALA | A | 203 | 25.110 | 61.705 | 3.525 | 1.00 | 44.40 |
| ATOM | 1615 | O | ALA | A | 203 | 25.924 | 61.159 | 2.787 | 1.00 | 45.26 |
| ATOM | 1616 | N | ALA | A | 204 | 24.920 | 61.348 | 4.788 | 1.00 | 47.23 |
| ATOM | 1617 | CA | ALA | A | 204 | 25.733 | 60.316 | 5.408 | 1.00 | 47.81 |
| ATOM | 1618 | Q | ALA | A | 204 | 25.266 | 60.089 | 6.817 | 1.00 | 48.23 |
| ATOM | 1619 | C | ALA | A | 204 | 25.701 | 59.000 | 4.615 | 1.00 | 48.73 |
| ATOM | 1620 | O | ALA | A | 204 | 26.680 | 58.252 | 4.581 | 1.00 | 51.77 |
| ATOM | 1621 | N | ASP | A | 205 | 24.574 | 58.725 | 3.970 | 1.00 | 45.72 |
| ATOM | 1622 | CA | ASP | A | 205 | 24.437 | 57.501 | 3.189 | 1.00 | 42.78 |
| ATOM | 1623 | CB | ASP | A | 205 | 22.984 | 56.989 | 3.221 | 1.00 | 47.36 |
| ATOM | 1624 | CG | ASP | A | 205 | 22.018 | 57.827 | 2.370 | 1.00 | 50.09 |
| ATOM | 1625 | OD1 | ASP | A | 205 | 22.374 | 58.937 | 1.922 | 1.00 | 54.80 |
| ATOM | 1626 | OD2 | ASP | A | 205 | 20.880 | 57.362 | 2.147 | 1.00 | 48.85 |
| ATOM | 1627 | C | ASP | A | 205 | 24.915 | 57.644 | 1.751 | 1.00 | 39.10 |
| ATOM | 1628 | O | ASP | A | 205 | 24.628 | 56.787 | 0.924 | 1.00 | 39.36 |
| ATOM | 1629 | N | GLY | A | 206 | 25.597 | 58.744 | 1.447 | 1.00 | 37.66 |
| ATOM | 1630 | CA | GLY | A | 206 | 26.100 | 58.968 | 0.097 | 1.00 | 36.43 |
| ATOM | 1631 | C | GLY | A | 206 | 25.238 | 59.669 | −0.950 | 1.00 | 34.15 |
| ATOM | 1632 | O | GLY | A | 206 | 25.739 | 60.024 | −2.017 | 1.00 | 31.94 |
| ATOM | 1633 | N | THR | A | 207 | 23.956 | 59.877 | −0.679 | 1.00 | 29.49 |
| ATOM | 1634 | CA | THR | A | 207 | 23.109 | 60.538 | −1.657 | 1.00 | 25.87 |
| ATOM | 1635 | CB | THR | A | 207 | 21.637 | 60.442 | −1.260 | 1.00 | 27.03 |
| ATOM | 1636 | OG1 | THR | A | 207 | 21.345 | 59.109 | −0.824 | 1.00 | 29.78 |
| ATOM | 1637 | CG2 | THR | A | 207 | 20.766 | 60.752 | −2.447 | 1.00 | 28.36 |
| ATOM | 1638 | C | THR | A | 207 | 23.509 | 61.998 | −1.837 | 1.00 | 25.47 |
| ATOM | 1639 | O | THR | A | 207 | 23.891 | 62.663 | −0.881 | 1.00 | 27.07 |
| ATOM | 1640 | N | LYS | A | 208 | 23.481 | 62.478 | −3.073 | 1.00 | 26.75 |
| ATOM | 1641 | CA | LYS | A | 208 | 23.828 | 63.866 | −3.347 | 1.00 | 28.57 |
| ATOM | 1642 | Q | LYS | A | 208 | 24.323 | 64.035 | −4.785 | 1.00 | 34.36 |
| ATOM | 1643 | CG | LYS | A | 208 | 25.565 | 63.216 | −5.112 | 1.00 | 40.54 |
| ATOM | 1644 | CD | LYS | A | 208 | 26.734 | 63.592 | −4.210 | 1.00 | 53.48 |
| ATOM | 1645 | CE | LYS | A | 208 | 27.937 | 62.669 | −4.416 | 1.00 | 58.70 |
| ATOM | 1646 | NZ | LYS | A | 208 | 29.114 | 63.071 | −3.586 | 1.00 | 62.46 |
| ATOM | 1647 | C | LYS | A | 208 | 22.540 | 64.626 | −3.124 | 1.00 | 27.85 |
| ATOM | 1648 | O | LYS | A | 208 | 21.497 | 64.231 | −3.638 | 1.00 | 29.27 |
| ATOM | 1649 | N | LEU | A | 209 | 22.620 | 65.743 | −2.413 | 1.00 | 25.63 |
| ATOM | 1650 | CA | LEU | A | 209 | 21.442 | 66.518 | −2.066 | 1.00 | 18.54 |
| ATOM | 1651 | CB | LEU | A | 209 | 21.149 | 66.348 | −0.583 | 1.00 | 15.76 |
| ATOM | 1652 | CG | LEU | A | 209 | 21.027 | 64.949 | −0.009 | 1.00 | 16.54 |
| ATOM | 1653 | CD1 | LEU | A | 209 | 21.176 | 65.029 | 1.478 | 1.00 | 19.47 |
| ATOM | 1654 | CD2 | LEU | A | 209 | 19.704 | 64.330 | −0.393 | 1.00 | 14.83 |
| ATOM | 1655 | C | LEU | A | 209 | 21.570 | 67.997 | −2.303 | 1.00 | 19.56 |
| ATOM | 1656 | O | LEU | A | 209 | 22.647 | 68.561 | −2.184 | 1.00 | 20.20 |
| ATOM | 1657 | N | SER | A | 210 | 20.438 | 68.624 | −2.591 | 1.00 | 20.72 |
| ATOM | 1658 | CA | SER | A | 210 | 20.359 | 70.064 | −2.782 | 1.00 | 26.32 |
| ATOM | 1659 | CA | SER | A | 210 | 19.243 | 70.433 | −3.770 | 1.00 | 26.58 |
| ATOM | 1660 | OG | SER | A | 210 | 19.611 | 70.186 | −5.115 | 1.00 | 34.59 |
| ATOM | 1661 | C | SER | A | 210 | 20.000 | 70.625 | −1.407 | 1.00 | 26.44 |
| ATOM | 1662 | O | SER | A | 210 | 20.466 | 71.694 | −1.023 | 1.00 | 29.35 |
| ATOM | 1663 | N | PHE | A | 211 | 19.143 | 69.900 | −0.683 | 1.00 | 27.04 |
| ATOM | 1664 | CA | PHE | A | 211 | 18.691 | 70.296 | 0.652 | 1.00 | 24.33 |
| ATOM | 1665 | CB | PHE | A | 211 | 17.306 | 70.938 | 0.603 | 1.00 | 25.32 |
| ATOM | 1666 | CG | PHE | A | 211 | 17.275 | 72.243 | −0.123 | 1.00 | 27.69 |
| ATOM | 1667 | CD1 | PHE | A | 211 | 16.682 | 72.341 | −1.378 | 1.00 | 35.01 |
| ATOM | 1668 | CD2 | PHE | A | 211 | 17.867 | 73.370 | 0.426 | 1.00 | 31.73 |
| ATOM | 1669 | CE1 | PHE | A | 211 | 16.681 | 73.547 | −2.082 | 1.00 | 35.58 |
| ATOM | 1670 | CE2 | PHE | A | 211 | 17.875 | 74.586 | −0.269 | 1.00 | 34.04 |
| ATOM | 1671 | CZ | PHE | A | 211 | 17.281 | 74.672 | −1.525 | 1.00 | 36.38 |
| ATOM | 1672 | C | PHE | A | 211 | 18.670 | 69.116 | 1.611 | 1.00 | 24.35 |
| ATOM | 1673 | O | PHE | A | 211 | 18.062 | 68.076 | 1.339 | 1.00 | 21.15 |
| ATOM | 1674 | N | GLU | A | 212 | 19.354 | 69.304 | 2.732 | 1.00 | 25.04 |
| ATOM | 1675 | CA | GLU | A | 212 | 19.485 | 68.311 | 3.777 | 1.00 | 26.00 |
| ATOM | 1676 | CB | GLU | A | 212 | 20.580 | 68.763 | 4.742 | 1.00 | 31.89 |
| ATOM | 1677 | CG | GLU | A | 212 | 21.004 | 67.726 | 5.770 | 1.00 | 52.30 |
| ATOM | 1678 | CD | GLU | A | 212 | 21.606 | 66.472 | 5.145 | 1.00 | 60.61 |
| ATOM | 1679 | OE1 | GLU | A | 212 | 20.903 | 65.434 | 5.092 | 1.00 | 59.97 |
| ATOM | 1680 | OE2 | GLU | A | 212 | 22.786 | 66.527 | 4.718 | 1.00 | 67.14 |
| ATOM | 1681 | C | GLU | A | 212 | 18.158 | 68.100 | 4.500 | 1.00 | 24.80 |
| ATOM | 1682 | O | GLU | A | 212 | 17.243 | 68.918 | 4.392 | 1.00 | 22.54 |
| ATOM | 1683 | N | TRP | A | 213 | 18.059 | 66.997 | 5.234 | 1.00 | 26.64 |
| ATOM | 1684 | CA | TRP | A | 213 | 16.846 | 66.653 | 5.963 | 1.00 | 28.18 |
| ATOM | 1685 | CB | TRP | A | 213 | 17.062 | 65.393 | 6.811 | 1.00 | 27.08 |
| ATOM | 1686 | CG | TRP | A | 213 | 17.942 | 65.589 | 7.993 | 1.00 | 31.90 |
| ATOM | 1687 | CD2 | TRP | A | 213 | 17.541 | 66.016 | 9.295 | 1.00 | 34.81 |
| ATOM | 1688 | CE2 | TRP | A | 213 | 18.702 | 66.070 | 10.093 | 1.00 | 39.48 |
| ATOM | 1689 | CE3 | TRP | A | 213 | 16.310 | 66.353 | 9.867 | 1.00 | 35.14 |
| ATOM | 1690 | CD1 | TRP | A | 213 | 19.291 | 65.406 | 8.051 | 1.00 | 34.70 |
| ATOM | 1691 | NE1 | TRP | A | 213 | 19.759 | 65.698 | 9.306 | 1.00 | 38.91 |
| ATOM | 1692 | CZ2 | TRP | A | 213 | 18.671 | 66.456 | 11.436 | 1.00 | 39.22 |
| ATOM | 1693 | CZ3 | TRP | A | 213 | 16.278 | 66.736 | 11.198 | 1.00 | 36.20 |
| ATOM | 1694 | CH2 | TRP | A | 213 | 17.452 | 66.780 | 11.969 | 1.00 | 39.04 |
| ATOM | 1695 | C | TRP | A | 213 | 16.312 | 67.780 | 6.840 | 1.00 | 27.32 |
| ATOM | 1696 | O | TRP | A | 213 | 17.074 | 68.601 | 7.341 | 1.00 | 26.78 |
| ATOM | 1697 | N | HIS | A | 214 | 14.994 | 67.785 | 7.033 | 1.00 | 25.99 |
| ATOM | 1698 | CA | HIS | A | 214 | 14.312 | 68.785 | 9.843 | 1.00 | 21.26 |
| ATOM | 1699 | CB | HIS | A | 214 | 14.498 | 70.170 | 7.229 | 1.00 | 24.00 |
| ATOM | 1700 | CG | HIS | A | 214 | 14.011 | 70.268 | 5.815 | 1.00 | 26.31 |
| ATOM | 1701 | CD2 | HIS | A | 214 | 12.986 | 70.964 | 5.265 | 1.00 | 20.40 |
| ATOM | 1702 | ND1 | HIS | A | 214 | 14.604 | 69.575 | 4.782 | 1.00 | 24.70 |
| ATOM | 1703 | CE1 | HIS | A | 214 | 13.966 | 69.840 | 3.657 | 1.00 | 19.84 |
| ATOM | 1704 | NE2 | HIS | A | 214 | 12.983 | 70.682 | 3.921 | 1.00 | 19.56 |
| ATOM | 1705 | C | HIS | A | 214 | 12.824 | 68.508 | 7.915 | 1.00 | 20.75 |
| ATOM | 1706 | O | HIS | A | 214 | 12.295 | 67.628 | 7.230 | 1.00 | 22.27 |
| ATOM | 1707 | N | GLU | A | 215 | 12.153 | 69.316 | 8.718 | 1.00 | 21.29 |
| ATOM | 1708 | Q | GLU | A | 215 | 10.710 | 69.258 | 8.888 | 1.00 | 25.72 |
| ATOM | 1709 | CB | GLU | A | 215 | 10.347 | 68.937 | 10.341 | 1.00 | 27.88 |
| ATOM | 1710 | CG | GLU | A | 215 | 11.325 | 69.505 | 11.344 | 1.00 | 44.01 |
| ATOM | 1711 | CD | GLU | A | 215 | 10.962 | 69.188 | 12.773 | 1.00 | 49.99 |
| ATOM | 1712 | OE1 | GLU | A | 215 | 10.532 | 68.043 | 13.040 | 1.00 | 48.65 |
| ATOM | 1713 | OE2 | GLU | A | 215 | 11.118 | 70.089 | 13.628 | 1.00 | 56.84 |
| ATOM | 1714 | C | GLU | A | 215 | 10.320 | 70.676 | 8.504 | 1.00 | 23.84 |
| ATOM | 1715 | O | GLU | A | 215 | 11.116 | 71.595 | 8.672 | 1.00 | 24.88 |
| ATOM | 1716 | N | ASP | A | 216 | 9.136 | 70.858 | 7.935 | 1.00 | 22.73 |
| ATOM | 1717 | CA | ASP | A | 216 | 8.732 | 72.187 | 7.492 | 1.00 | 22.94 |
| ATOM | 1718 | CB | ASP | A | 216 | 7.636 | 72.112 | 6.407 | 1.00 | 27.97 |
| ATOM | 1719 | CG | ASP | A | 216 | 8.082 | 71.398 | 5.135 | 1.00 | 27.71 |
| ATOM | 1720 | OD1 | ASP | A | 216 | 9.304 | 71.232 | 4.918 | 1.00 | 31.88 |
| ATOM | 1721 | OD2 | ASP | A | 216 | 7.185 | 71.015 | 4.344 | 1.00 | 26.49 |
| ATOM | 1722 | C | ASP | A | 216 | 8.230 | 73.096 | 8.596 | 1.00 | 19.73 |
| ATOM | 1723 | O | ASP | A | 216 | 7.680 | 72.652 | 9.594 | 1.00 | 23.16 |
| ATOM | 1724 | N | VAL | A | 217 | 8.433 | 74.384 | 8.398 | 1.00 | 16.59 |
| ATOM | 1725 | CA | VAL | A | 217 | 7.945 | 75.376 | 9.312 | 1.00 | 18.55 |
| ATOM | 1726 | CB | VAL | A | 217 | 8.907 | 76.564 | 9.424 | 1.00 | 16.49 |
| ATOM | 1727 | CG1 | VAL | A | 217 | 8.265 | 77.687 | 10.235 | 1.00 | 10.45 |
| ATOM | 1728 | CG2 | VAL | A | 217 | 10.179 | 76.123 | 10.088 | 1.00 | 11.70 |
| ATOM | 1729 | C | VAL | A | 217 | 6.652 | 75.819 | 8.633 | 1.00 | 23.10 |
| ATOM | 1730 | O | VAL | A | 217 | 6.667 | 76.674 | 7.729 | 1.00 | 21.60 |
| ATOM | 1731 | N | SER | A | 218 | 5.562 | 75.142 | 8.990 | 1.00 | 25.06 |
| ATOM | 1732 | CA | SER | A | 218 | 4.233 | 75.433 | 8.452 | 1.00 | 24.44 |
| ATOM | 1733 | CB | SER | A | 218 | 4.183 | 75.110 | 6.954 | 1.00 | 23.25 |
| ATOM | 1734 | OG | SER | A | 218 | 4.109 | 73.706 | 6.717 | 1.00 | 16.35 |
| ATOM | 1735 | C | SER | A | 218 | 3.190 | 74.585 | 9.178 | 1.00 | 23.44 |
| ATOM | 1736 | O | SER | A | 218 | 3.541 | 73.734 | 9.996 | 1.00 | 23.65 |
| ATOM | 1737 | N | LEU | A | 219 | 1.913 | 74.865 | 8.932 | 1.00 | 21.85 |
| ATOM | 1738 | CA | LEU | A | 219 | 0.847 | 74.064 | 9.518 | 1.00 | 22.52 |
| ATOM | 1739 | CB | LEU | A | 219 | −0.493 | 74.797 | 9.483 | 1.00 | 22.11 |
| ATOM | 1740 | CG | LEU | A | 219 | −1.687 | 73.960 | 9.955 | 1.00 | 18.98 |
| ATOM | 1741 | CD1 | LEU | A | 219 | −1.427 | 73.419 | 11.330 | 1.00 | 17.47 |
| ATOM | 1742 | CD2 | LEU | A | 219 | −2.933 | 74.804 | 9.956 | 1.00 | 19.86 |
| ATOM | 1743 | C | LEU | A | 219 | 0.822 | 72.817 | 8.633 | 1.00 | 24.74 |
| ATOM | 1744 | O | LEU | A | 219 | 0.870 | 71.697 | 9.128 | 1.00 | 26.48 |
| ATOM | 1745 | N | ILE | A | 220 | 0.760 | 73.030 | 7.318 | 1.00 | 24.79 |
| ATOM | 1746 | CA | ILE | A | 220 | 0.821 | 71.960 | 6.309 | 1.00 | 22.56 |
| ATOM | 1747 | CB | ILE | A | 220 | −0.563 | 71.393 | 5.860 | 1.00 | 21.63 |
| ATOM | 1748 | CG2 | ILE | A | 220 | −1.335 | 70.840 | 7.040 | 1.00 | 20.63 |
| ATOM | 1749 | CG1 | ILE | A | 220 | −1.380 | 72.443 | 5.109 | 1.00 | 22.42 |
| ATOM | 1750 | CD1 | ILE | A | 220 | −2.505 | 71.841 | 4.297 | 1.00 | 18.13 |
| ATOM | 1751 | C | ILE | A | 220 | 1.501 | 72.600 | 5.099 | 1.00 | 18.56 |
| ATOM | 1752 | O | ILE | A | 220 | 1.496 | 73.825 | 4.970 | 1.00 | 17.45 |
| ATOM | 1753 | N | THR | A | 221 | 2.141 | 71.793 | 4.263 | 1.00 | 17.74 |
| ATOM | 1754 | CA | THR | A | 221 | 2.802 | 72.295 | 3.069 | 1.00 | 14.91 |
| ATOM | 1755 | CB | THR | A | 221 | 4.287 | 71.939 | 3.032 | 1.00 | 15.95 |
| ATOM | 1756 | OG1 | THR | A | 221 | 4.937 | 72.557 | 4.146 | 1.00 | 18.73 |
| ATOM | 1757 | CG2 | THR | A | 221 | 4.928 | 72.469 | 1.773 | 1.00 | 10.41 |
| ATOM | 1758 | C | THR | A | 221 | 2.066 | 71.651 | 1.916 | 1.00 | 16.30 |
| ATOM | 1759 | O | THR | A | 221 | 1.711 | 70.477 | 1.983 | 1.00 | 18.87 |
| ATOM | 1760 | N | VAL | A | 222 | 1.729 | 72.477 | 0.933 | 1.00 | 16.62 |
| ATOM | 1761 | CA | VAL | A | 222 | 0.992 | 72.088 | −0.261 | 1.00 | 17.81 |
| ATOM | 1762 | CB | VAL | A | 222 | −0.332 | 72.880 | −0.310 | 1.00 | 22.05 |
| ATOM | 1763 | CG1 | VAL | A | 222 | −1.046 | 72.671 | −1.630 | 1.00 | 25.61 |
| ATOM | 1764 | CG2 | VAL | A | 222 | −1.221 | 72.446 | 0.854 | 1.00 | 24.18 |
| ATOM | 1765 | C | VAL | A | 222 | 1.890 | 72.404 | −1.464 | 1.00 | 17.84 |
| ATOM | 1766 | O | VAL | A | 222 | 1.990 | 73.557 | −1.895 | 1.00 | 18.42 |
| ATOM | 1767 | N | LEU | A | 223 | 2.525 | 71.359 | −1.995 | 1.00 | 14.96 |
| ATOM | 1768 | CA | LEU | A | 223 | 3.495 | 71.469 | −3.080 | 1.00 | 15.97 |
| ATOM | 1769 | CB | LEU | A | 223 | 4.779 | 70.761 | −2.647 | 1.00 | 8.17 |
| ATOM | 1770 | CG | LEU | A | 223 | 5.836 | 70.407 | −3.680 | 1.00 | 9.19 |
| ATOM | 1771 | CD1 | LEU | A | 223 | 6.771 | 71.557 | −3.882 | 1.00 | 9.69 |
| ATOM | 1772 | CD2 | LEU | A | 223 | 6.605 | 69.205 | −3.191 | 1.00 | 12.71 |
| ATOM | 1773 | C | LEU | A | 223 | 3.146 | 70.951 | −4.461 | 1.00 | 20.09 |
| ATOM | 1774 | O | LEU | A | 223 | 2.623 | 69.846 | −4.608 | 1.00 | 24.25 |
| ATOM | 1775 | N | TYR | A | 224 | 3.453 | 71.757 | −5.476 | 1.00 | 23.92 |
| ATOM | 1776 | CA | TYR | A | 224 | 3.283 | 71.340 | −6.862 | 1.00 | 22.47 |
| ATOM | 1777 | CB | TYR | A | 224 | 2.415 | 72.269 | −7.711 | 1.00 | 25.33 |
| ATOM | 1778 | CG | TYR | A | 224 | 2.258 | 71.701 | −9.110 | 1.00 | 24.10 |
| ATOM | 1779 | CD1 | TYR | A | 224 | 1.592 | 70.488 | −9.311 | 1.00 | 22.12 |
| ATOM | 1780 | CE1 | TYR | A | 224 | 1.534 | 69.893 | −10.560 | 1.00 | 25.03 |
| ATOM | 1781 | CD2 | TYR | A | 224 | 2.860 | 72.311 | −10.212 | 1.00 | 23.73 |
| ATOM | 1782 | CE2 | TYR | A | 224 | 2.811 | 71.720 | −11.475 | 1.00 | 27.76 |
| ATOM | 1783 | CZ | TYR | A | 224 | 2.146 | 70.505 | −11.643 | 1.00 | 29.83 |
| ATOM | 1784 | OH | TYR | A | 224 | 2.112 | 69.895 | −12.884 | 1.00 | 31.58 |
| ATOM | 1785 | C | TYR | A | 224 | 4.680 | 71.314 | −7.451 | 1.00 | 25.24 |
| ATOM | 1786 | O | TYR | A | 224 | 5.424 | 72.301 | −7.392 | 1.00 | 27.50 |
| ATOM | 1787 | N | GLN | A | 225 | 5.014 | 70.193 | −8.060 | 1.00 | 23.63 |
| ATOM | 1788 | CA | GLN | A | 225 | 6.327 | 70.006 | −8.639 | 1.00 | 25.73 |
| ATOM | 1789 | Q | GLN | A | 225 | 7.184 | 69.294 | −7.609 | 1.00 | 23.37 |
| ATOM | 1790 | CG | GLN | A | 225 | 8.614 | 69.292 | −7.891 | 1.00 | 25.23 |
| ATOM | 1791 | CD | GLN | A | 225 | 9.378 | 68.781 | −6.720 | 1.00 | 30.69 |
| ATOM | 1792 | OE1 | GLN | A | 225 | 10.264 | 69.461 | −6.203 | 1.00 | 37.77 |
| ATOM | 1793 | NE2 | GLN | A | 225 | 9.042 | 67.578 | −6.278 | 1.00 | 29.82 |
| ATOM | 1794 | C | GLN | A | 225 | 6.105 | 69.126 | −9.858 | 1.00 | 27.45 |
| ATOM | 1795 | O | GLN | A | 225 | 5.503 | 68.056 | −9.759 | 1.00 | 27.59 |
| ATOM | 1796 | N | SER | A | 226 | 6.601 | 69.552 | −11.007 | 1.00 | 27.34 |
| ATOM | 1797 | CA | SER | A | 226 | 6.358 | 68.779 | −12.210 | 1.00 | 28.99 |
| ATOM | 1798 | CB | SER | A | 226 | 5.501 | 69.605 | −13.152 | 1.00 | 28.18 |
| ATOM | 1799 | OG | SER | A | 226 | 5.052 | 68.829 | −14.229 | 1.00 | 40.16 |
| ATOM | 1800 | C | SER | A | 226 | 7.599 | 68.311 | −12.943 | 1.00 | 31.56 |
| ATOM | 1801 | O | SER | A | 226 | 8.570 | 69.055 | −13.087 | 1.00 | 32.05 |
| ATOM | 1802 | N | ASN | A | 227 | 7.571 | 67.055 | −13.370 | 1.00 | 30.89 |
| ATOM | 1803 | CA | ASN | A | 227 | 8.667 | 66.471 | −14.133 | 1.00 | 29.18 |
| ATOM | 1804 | CB | ASN | A | 227 | 8.878 | 67.285 | −15.417 | 1.00 | 28.72 |
| ATOM | 1805 | CG | ASN | A | 227 | 9.511 | 66.482 | −16.528 | 1.00 | 27.74 |
| ATOM | 1806 | OD1 | ASN | A | 227 | 9.130 | 65.341 | −16.779 | 1.00 | 28.75 |
| ATOM | 1807 | ND2 | ASN | A | 227 | 10.454 | 67.089 | −17.229 | 1.00 | 27.94 |
| ATOM | 1808 | C | ASN | A | 227 | 9.990 | 66.328 | −13.380 | 1.00 | 28.97 |
| ATOM | 1809 | O | ASN | A | 227 | 11.052 | 66.517 | −13.960 | 1.00 | 30.10 |
| ATOM | 1810 | N | VAL | A | 228 | 9.929 | 66.006 | −12.091 | 1.00 | 29.20 |
| ATOM | 1811 | CA | VAL | A | 228 | 11.135 | 65.809 | −11.291 | 1.00 | 27.27 |
| ATOM | 1812 | CB | VAL | A | 228 | 11.599 | 67.058 | −10.497 | 1.00 | 26.29 |
| ATOM | 1813 | CG1 | VAL | A | 228 | 13.083 | 67.241 | −10.649 | 1.00 | 23.43 |
| ATOM | 1814 | CG2 | VAL | A | 228 | 10.827 | 68.292 | −10.859 | 1.00 | 25.97 |
| ATOM | 1815 | C | VAL | A | 228 | 10.799 | 64.826 | −10.206 | 1.00 | 31.99 |
| ATOM | 1816 | O | VAL | A | 228 | 9.651 | 64.760 | −9.758 | 1.00 | 35.97 |
| ATOM | 1817 | N | GLN | A | 229 | 11.822 | 64.110 | −9.753 | 1.00 | 34.06 |
| ATOM | 1818 | CA | GLN | A | 229 | 11.715 | 63.153 | −8.658 | 1.00 | 33.36 |
| ATOM | 1819 | CB | GLN | A | 229 | 11.795 | 61.734 | −9.176 | 1.00 | 37.53 |
| ATOM | 1820 | CG | GLN | A | 229 | 11.559 | 60.666 | −8.148 | 1.00 | 42.23 |
| ATOM | 1821 | CD | GLN | A | 229 | 11.207 | 59.368 | −8.833 | 1.00 | 55.26 |
| ATOM | 1822 | OE1 | GLN | A | 229 | 10.110 | 59.222 | −9.392 | 1.00 | 58.46 |
| ATOM | 1823 | NE2 | GLN | A | 229 | 12.155 | 58.436 | −8.857 | 1.00 | 59.25 |
| ATOM | 1824 | C | GLN | A | 229 | 12.935 | 63.474 | −7.814 | 1.00 | 34.22 |
| ATOM | 1825 | O | GLN | A | 229 | 14.044 | 63.042 | −8.124 | 1.00 | 34.78 |
| ATOM | 1826 | N | ASN | A | 230 | 12.733 | 64.293 | −6.784 | 1.00 | 34.28 |
| ATOM | 1827 | CA | ASN | A | 230 | 13.827 | 64.732 | −5.917 | 1.00 | 32.42 |
| ATOM | 1828 | CB | ASN | A | 230 | 14.171 | 66.199 | −6.226 | 1.00 | 27.53 |
| ATOM | 1829 | CG | ASN | A | 230 | 12.974 | 67.136 | −6.068 | 1.00 | 21.96 |
| ATOM | 1830 | OD1 | ASN | A | 230 | 11.933 | 66.751 | −5.545 | 1.00 | 24.46 |
| ATOM | 1831 | ND2 | ASN | A | 230 | 13.118 | 68.361 | −6.541 | 1.00 | 19.87 |
| ATOM | 1832 | C | ASN | A | 230 | 13.575 | 64.587 | −4.419 | 1.00 | 31.95 |
| ATOM | 1833 | O | ASN | A | 230 | 14.476 | 64.808 | −3.622 | 1.00 | 30.47 |
| ATOM | 1834 | N | LEU | A | 231 | 12.356 | 64.231 | −4.034 | 1.00 | 31.28 |
| ATOM | 1835 | CA | LEU | A | 231 | 12.018 | 64.092 | −2.626 | 1.00 | 27.98 |
| ATOM | 1836 | CB | LEU | A | 231 | 10.551 | 64.431 | −2.408 | 1.00 | 24.65 |
| ATOM | 1837 | CG | LEU | A | 231 | 10.254 | 65.884 | −2.135 | 1.00 | 21.29 |
| ATOM | 1838 | CD1 | LEU | A | 231 | 8.807 | 65.985 | −1.745 | 1.00 | 24.61 |
| ATOM | 1839 | CD2 | LEU | A | 231 | 11.116 | 66.349 | −0.993 | 1.00 | 23.04 |
| ATOM | 1840 | C | LEU | A | 231 | 12.276 | 62.719 | −2.040 | 1.00 | 30.04 |
| ATOM | 1841 | O | LEU | A | 231 | 12.145 | 61.706 | −2.725 | 1.00 | 34.74 |
| ATOM | 1842 | N | GLN | A | 232 | 12.615 | 62.693 | −0.754 | 1.00 | 30.14 |
| ATOM | 1843 | CA | GLN | A | 232 | 12.834 | 61.415 | −0.034 | 1.00 | 26.30 |
| ATOM | 1844 | CB | GLN | A | 232 | 14.314 | 61.087 | 0.000 | 1.00 | 25.18 |
| ATOM | 1845 | CG | GLN | A | 232 | 14.877 | 60.607 | −1.315 | 1.00 | 24.42 |
| ATOM | 1846 | CD | GLN | A | 232 | 16.251 | 59.976 | −1.163 | 1.00 | 28.50 |
| ATOM | 1847 | OE1 | GLN | A | 232 | 17.149 | 60.543 | −0.538 | 1.00 | 29.12 |
| ATOM | 1848 | NE2 | GLN | A | 232 | 16.420 | 58.794 | −1.736 | 1.00 | 27.79 |
| ATOM | 1849 | C | GLN | A | 232 | 12.313 | 61.614 | 1.392 | 1.00 | 27.21 |
| ATOM | 1850 | O | GLN | A | 232 | 12.538 | 62.656 | 2.015 | 1.00 | 29.07 |
| ATOM | 1851 | N | VAL | A | 233 | 11.581 | 60.618 | 1.888 | 1.00 | 25.56 |
| ATOM | 1852 | CA | VAL | A | 233 | 11.047 | 60.659 | 3.250 | 1.00 | 23.90 |
| ATOM | 1853 | CB | VAL | A | 233 | 9.588 | 60.195 | 3.354 | 1.00 | 26.06 |
| ATOM | 1854 | CG1 | VAL | A | 233 | 8.862 | 61.027 | 4.382 | 1.00 | 28.07 |
| ATOM | 1855 | CG2 | VAL | A | 233 | 8.911 | 60.178 | 2.027 | 1.00 | 25.39 |
| ATOM | 1856 | C | VAL | A | 233 | 11.779 | 59.644 | 4.101 | 1.00 | 27.04 |
| ATOM | 1857 | O | VAL | A | 233 | 12.024 | 58.519 | 3.662 | 1.00 | 27.08 |
| ATOM | 1858 | N | GLU | A | 234 | 12.090 | 60.015 | 5.331 | 1.00 | 27.08 |
| ATOM | 1859 | CA | GLU | A | 234 | 12.744 | 59.089 | 6.226 | 1.00 | 28.63 |
| ATOM | 1860 | CB | GLU | A | 234 | 13.512 | 59.848 | 7.289 | 1.00 | 28.65 |
| ATOM | 1861 | CG | GLU | A | 234 | 14.044 | 58.977 | 8.400 | 1.00 | 33.81 |
| ATOM | 1862 | CD | GLU | A | 234 | 14.652 | 59.788 | 9.509 | 1.00 | 37.83 |
| ATOM | 1863 | OE1 | GLU | A | 234 | 15.893 | 59.870 | 9.558 | 1.00 | 46.49 |
| ATOM | 1864 | OE2 | GLU | A | 234 | 13.894 | 60.356 | 10.323 | 1.00 | 41.82 |
| ATOM | 1865 | C | GLU | A | 234 | 11.637 | 58.292 | 6.881 | 1.00 | 32.99 |
| ATOM | 1866 | O | GLU | A | 234 | 10.761 | 58.861 | 7.526 | 1.00 | 36.86 |
| ATOM | 1867 | N | THR | A | 235 | 11.619 | 56.990 | 6.654 | 1.00 | 39.05 |
| ATOM | 1868 | CA | THR | A | 235 | 10.603 | 56.144 | 7.264 | 1.00 | 43.64 |
| ATOM | 1869 | CB | THR | A | 235 | 9.789 | 55.370 | 6.196 | 1.00 | 47.64 |
| ATOM | 1870 | OG1 | THR | A | 235 | 10.663 | 54.518 | 5.443 | 1.00 | 48.96 |
| ATOM | 1871 | CG2 | THR | A | 235 | 9.077 | 56.340 | 5.245 | 1.00 | 49.04 |
| ATOM | 1872 | C | THR | A | 235 | 11.310 | 55.161 | 8.186 | 1.00 | 45.27 |
| ATOM | 1873 | O | THR | A | 235 | 12.533 | 55.204 | 8.330 | 1.00 | 46.15 |
| ATOM | 1874 | N | ALA | A | 236 | 10.549 | 54.266 | 8.802 | 1.00 | 48.85 |
| ATOM | 1875 | CA | ALA | A | 236 | 11.131 | 53.271 | 9.697 | 1.00 | 51.37 |
| ATOM | 1876 | CB | ALA | A | 236 | 10.035 | 52.506 | 10.416 | 1.00 | 52.57 |
| ATOM | 1877 | C | ALA | A | 236 | 12.049 | 52.307 | 8.944 | 1.00 | 51.92 |
| ATOM | 1878 | O | ALA | A | 236 | 12.709 | 51.464 | 9.547 | 1.00 | 56.47 |
| ATOM | 1879 | N | ALA | A | 237 | 12.044 | 52.402 | 7.620 | 1.00 | 50.60 |
| ATOM | 1880 | CA | ALA | A | 237 | 12.886 | 51.558 | 6.786 | 1.00 | 50.28 |
| ATOM | 1881 | CB | ALA | A | 237 | 12.044 | 50.870 | 5.720 | 1.00 | 45.48 |
| ATOM | 1882 | C | ALA | A | 237 | 13.952 | 52.421 | 6.126 | 1.00 | 51.53 |
| ATOM | 1883 | O | ALA | A | 237 | 14.591 | 51.992 | 5.156 | 1.00 | 53.02 |
| ATOM | 1884 | N | GLY | A | 238 | 14.159 | 53.620 | 6.672 | 1.00 | 49.22 |
| ATOM | 1885 | CA | GLY | A | 238 | 15.129 | 54.542 | 6.108 | 1.00 | 47.88 |
| ATOM | 1886 | C | GLY | A | 238 | 14.493 | 55.410 | 5.029 | 1.00 | 46.36 |
| ATOM | 1887 | O | GLY | A | 238 | 13.275 | 55.377 | 4.837 | 1.00 | 45.44 |
| ATOM | 1888 | N | TYR | A | 239 | 15.305 | 56.193 | 4.325 | 1.00 | 43.94 |
| ATOM | 1889 | CA | TYR | A | 239 | 14.795 | 57.077 | 3.282 | 1.00 | 42.31 |
| ATOM | 1890 | CB | TYR | A | 239 | 15.860 | 58.079 | 2.862 | 1.00 | 34.38 |
| ATOM | 1891 | CG | TYR | A | 239 | 16.054 | 59.203 | 3.846 | 1.00 | 31.86 |
| ATOM | 1892 | CD1 | TYR | A | 239 | 16.902 | 59.064 | 4.943 | 1.00 | 29.03 |
| ATOM | 1893 | CE1 | TYR | A | 239 | 17.115 | 60.129 | 5.825 | 1.00 | 30.21 |
| ATOM | 1894 | CD2 | TYR | A | 239 | 15.416 | 60.427 | 3.659 | 1.00 | 31.58 |
| ATOM | 1895 | CE2 | TYR | A | 239 | 15.618 | 61.491 | 4.536 | 1.00 | 27.77 |
| ATOM | 1896 | CZ | TYR | A | 239 | 16.467 | 61.337 | 5.609 | 1.00 | 28.27 |
| ATOM | 1897 | OH | TYR | A | 239 | 16.670 | 62.396 | 6.459 | 1.00 | 30.28 |
| ATOM | 1898 | C | TYR | A | 239 | 14.282 | 56.345 | 2.053 | 1.00 | 44.26 |
| ATOM | 1899 | O | TYR | A | 239 | 14.958 | 55.464 | 1.519 | 1.00 | 50.07 |
| ATOM | 1900 | N | GLN | A | 240 | 13.089 | 56.730 | 1.605 | 1.00 | 42.95 |
| ATOM | 1901 | CA | GLN | A | 240 | 12.457 | 56.135 | 0.434 | 1.00 | 37.56 |
| ATOM | 1902 | CB | GLN | A | 240 | 11.227 | 55.338 | 0.829 | 1.00 | 37.90 |
| ATOM | 1903 | CG | GLN | A | 240 | 11.492 | 54.283 | 1.867 | 1.00 | 39.00 |
| ATOM | 1904 | CD | GLN | A | 240 | 10.259 | 53.503 | 2.178 | 1.00 | 41.54 |
| ATOM | 1905 | OE1 | GLN | A | 240 | 9.361 | 53.975 | 2.876 | 1.00 | 42.44 |
| ATOM | 1906 | NE2 | GLN | A | 240 | 10.192 | 52.303 | 1.655 | 1.00 | 43.71 |
| ATOM | 1907 | C | GLN | A | 240 | 12.036 | 57.238 | −0.493 | 1.00 | 35.12 |
| ATOM | 1908 | O | GLN | A | 240 | 11.637 | 58.310 | −0.049 | 1.00 | 37.16 |
| ATOM | 1909 | N | ASP | A | 241 | 12.106 | 56.963 | −1.786 | 1.00 | 37.65 |
| ATOM | 1910 | CA | ASP | A | 241 | 11.757 | 57.937 | −2.807 | 1.00 | 33.97 |
| ATOM | 1911 | CB | ASP | A | 241 | 12.294 | 57.477 | −4.157 | 1.00 | 36.71 |
| ATOM | 1912 | CG | ASP | A | 241 | 13.768 | 57.725 | −4.305 | 1.00 | 40.53 |
| ATOM | 1913 | OD1 | ASP | A | 241 | 14.501 | 56.767 | −4.626 | 1.00 | 48.07 |
| ATOM | 1914 | OD2 | ASP | A | 241 | 14.193 | 58.883 | −4.102 | 1.00 | 43.60 |
| ATOM | 1915 | C | ASP | A | 241 | 10.273 | 58.187 | −2.937 | 1.00 | 31.87 |
| ATOM | 1916 | O | ASP | A | 241 | 9.473 | 57.290 | −2.715 | 1.00 | 33.89 |
| ATOM | 1917 | N | ILE | A | 242 | 9.910 | 59.420 | −3.270 | 1.00 | 29.50 |
| ATOM | 1918 | CA | ILE | A | 242 | 8.516 | 59.776 | −3.491 | 1.00 | 29.59 |
| ATOM | 1919 | CB | ILE | A | 242 | 8.122 | 61.110 | −2.793 | 1.00 | 25.82 |
| ATOM | 1920 | CG2 | ILE | A | 242 | 6.718 | 61.540 | −3.205 | 1.00 | 21.57 |
| ATOM | 1921 | CG1 | ILE | A | 242 | 8.142 | 60.940 | −1.275 | 1.00 | 22.34 |
| ATOM | 1922 | CD1 | ILE | A | 242 | 7.931 | 62.219 | −0.529 | 1.00 | 19.47 |
| ATOM | 1923 | C | ILE | A | 242 | 8.314 | 59.899 | −5.002 | 1.00 | 32.14 |
| ATOM | 1924 | O | ILE | A | 242 | 9.039 | 60.631 | −5.680 | 1.00 | 34.26 |
| ATOM | 1925 | N | GLU | A | 243 | 7.364 | 59.139 | −5.528 | 1.00 | 32.42 |
| ATOM | 1926 | CA | GLU | A | 243 | 7.051 | 59.161 | −6.950 | 1.00 | 35.87 |
| ATOM | 1927 | CB | GLU | A | 243 | 5.998 | 58.103 | −7.257 | 1.00 | 45.32 |
| ATOM | 1928 | CG | GLU | A | 243 | 4.620 | 58.422 | −6.675 | 1.00 | 58.52 |
| ATOM | 1929 | CD | GLU | A | 243 | 3.584 | 57.359 | −6.970 | 1.00 | 67.74 |
| ATOM | 1930 | OE1 | GLU | A | 243 | 2.679 | 57.157 | −6.126 | 1.00 | 72.36 |
| ATOM | 1931 | OE2 | GLU | A | 243 | 3.669 | 56.730 | −8.048 | 1.00 | 73.67 |
| ATOM | 1932 | C | GLU | A | 243 | 6.494 | 60.520 | −7.361 | 1.00 | 34.26 |
| ATOM | 1933 | O | GLU | A | 243 | 5.794 | 61.170 | −6.587 | 1.00 | 34.70 |
| ATOM | 1934 | N | ALA | A | 244 | 6.796 | 60.940 | −8.582 | 1.00 | 37.14 |
| ATOM | 1935 | CA | ALA | A | 244 | 6.299 | 62.211 | −9.106 | 1.00 | 36.70 |
| ATOM | 1936 | CB | ALA | A | 244 | 7.187 | 62.704 | −10.237 | 1.00 | 30.95 |
| ATOM | 1937 | C | ALA | A | 244 | 4.870 | 62.045 | −9.607 | 1.00 | 38.29 |
| ATOM | 1938 | O | ALA | A | 244 | 4.401 | 60.919 | −9.809 | 1.00 | 39.49 |
| ATOM | 1939 | N | ASP | A | 245 | 4.184 | 63.168 | −9.810 | 1.00 | 38.94 |
| ATOM | 1940 | CA | ASP | A | 245 | 2.809 | 63.171 | −10.302 | 1.00 | 36.20 |
| ATOM | 1941 | CB | ASP | A | 245 | 1.849 | 62.683 | −9.217 | 1.00 | 35.69 |
| ATOM | 1942 | CG | ASP | A | 245 | 0.436 | 62.479 | −9.730 | 1.00 | 39.12 |
| ATOM | 1943 | OD1 | ASP | A | 245 | −0.090 | 63.350 | −10.446 | 1.00 | 38.32 |
| ATOM | 1944 | OD2 | ASP | A | 245 | −0.162 | 61.440 | −9.406 | 1.00 | 44.34 |
| ATOM | 1945 | C | ASP | A | 245 | 2.423 | 64.579 | −10.720 | 1.00 | 36.64 |
| ATOM | 1946 | O | ASP | A | 245 | 1.881 | 65.338 | −9.920 | 1.00 | 40.34 |
| ATOM | 1947 | N | ASP | A | 246 | 2.624 | 64.906 | −11.989 | 1.00 | 36.70 |
| ATOM | 1948 | CA | ASP | A | 246 | 2.288 | 66.242 | −12.464 | 1.00 | 42.59 |
| ATOM | 1949 | CB | ASP | A | 246 | 2.956 | 66.546 | −13.815 | 1.00 | 52.74 |
| ATOM | 1950 | CG | ASP | A | 246 | 2.651 | 65.508 | −14.899 | 1.00 | 62.16 |
| ATOM | 1951 | OD1 | ASP | A | 246 | 3.484 | 65.398 | −15.834 | 1.00 | 67.79 |
| ATOM | 1952 | OD2 | ASP | A | 246 | 1.600 | 64.822 | −14.836 | 1.00 | 61.80 |
| ATOM | 1953 | C | ASP | A | 246 | 0.818 | 66.649 | −12.495 | 1.00 | 39.27 |
| ATOM | 1954 | O | ASP | A | 246 | 0.485 | 67.699 | −13.042 | 1.00 | 39.09 |
| ATOM | 1955 | N | THR | A | 247 | −0.058 | 65.849 | −11.897 | 1.00 | 37.20 |
| ATOM | 1956 | CA | THR | A | 247 | −1.479 | 66.182 | −11.876 | 1.00 | 37.88 |
| ATOM | 1957 | CB | THR | A | 247 | −2.345 | 65.074 | −12.545 | 1.00 | 41.31 |
| ATOM | 1958 | OG1 | THR | A | 247 | −2.370 | 63.901 | −11.719 | 1.00 | 44.54 |
| ATOM | 1959 | CG2 | THR | A | 247 | −1.770 | 64.688 | −13.903 | 1.00 | 42.77 |
| ATOM | 1960 | C | THR | A | 247 | −1.990 | 66.436 | −10.452 | 1.00 | 37.12 |
| ATOM | 1961 | O | THR | A | 247 | −3.109 | 66.933 | −10.263 | 1.00 | 35.84 |
| ATOM | 1962 | N | GLY | A | 248 | −1.162 | 66.136 | −9.453 | 1.00 | 33.34 |
| ATOM | 1963 | CA | GLY | A | 248 | −1.580 | 66.320 | −8.076 | 1.00 | 29.08 |
| ATOM | 1964 | C | GLY | A | 248 | −0.704 | 67.236 | −7.251 | 1.00 | 26.86 |
| ATOM | 1965 | O | GLY | A | 248 | 0.338 | 67.693 | −7.709 | 1.00 | 29.61 |
| ATOM | 1966 | N | TYR | A | 249 | −1.168 | 67.553 | −6.052 | 1.00 | 24.24 |
| ATOM | 1967 | CA | TYR | A | 249 | −0.432 | 68.401 | −5.128 | 1.00 | 22.26 |
| ATOM | 1968 | CB | TYR | A | 249 | −1.319 | 69.512 | −4.571 | 1.00 | 17.04 |
| ATOM | 1969 | CG | TYR | A | 249 | −1.337 | 70.775 | −5.395 | 1.00 | 19.97 |
| ATOM | 1970 | CD1 | TYR | A | 249 | −2.159 | 70.896 | −6.505 | 1.00 | 21.32 |
| ATOM | 1971 | CE1 | TYR | A | 249 | −2.194 | 72.078 | −7.247 | 1.00 | 22.14 |
| ATOM | 1972 | CD2 | TYR | A | 249 | −0.545 | 71.866 | −5.045 | 1.00 | 17.14 |
| ATOM | 1973 | CE2 | TYR | A | 249 | −0.575 | 73.041 | −5.777 | 1.00 | 18.05 |
| ATOM | 1974 | CZ | TYR | A | 249 | −1.398 | 73.140 | −6.876 | 1.00 | 18.13 |
| ATOM | 1975 | OH | TYR | A | 249 | −1.412 | 74.302 | −7.612 | 1.00 | 23.74 |
| ATOM | 1976 | C | TYR | A | 249 | 0.037 | 67.520 | −3.982 | 1.00 | 24.44 |
| ATOM | 1977 | O | TYR | A | 249 | −0.766 | 66.820 | −3.363 | 1.00 | 25.35 |
| ATOM | 1978 | N | LEU | A | 250 | 1.344 | 67.505 | −3.745 | 1.00 | 21.09 |
| ATOM | 1979 | CA | LEU | A | 250 | 1.908 | 66.720 | −2.664 | 1.00 | 19.72 |
| ATOM | 1980 | CB | LEU | A | 250 | 3.397 | 66.484 | −2.914 | 1.00 | 11.66 |
| ATOM | 1981 | CG | LEU | A | 250 | 4.092 | 65.533 | −1.946 | 1.00 | 13.51 |
| ATOM | 1982 | CD1 | LEU | A | 250 | 3.460 | 64.158 | −1.998 | 1.00 | 9.39 |
| ATOM | 1983 | CD2 | LEU | A | 250 | 5.536 | 65.450 | −2.310 | 1.00 | 16.26 |
| ATOM | 1984 | C | LEU | A | 250 | 1.683 | 67.475 | −1.349 | 1.00 | 24.50 |
| ATOM | 1985 | O | LEU | A | 250 | 2.160 | 68.606 | −1.176 | 1.00 | 23.74 |
| ATOM | 1986 | N | ILE | A | 251 | 0.953 | 66.847 | −0.432 | 1.00 | 25.00 |
| ATOM | 1987 | CA | ILE | A | 251 | 0.651 | 67.447 | 0.866 | 1.00 | 23.54 |
| ATOM | 1988 | CB | ILE | A | 251 | −0.876 | 67.411 | 1.138 | 1.00 | 21.64 |
| ATOM | 1989 | CG2 | ILE | A | 251 | −1.257 | 68.522 | 2.121 | 1.00 | 20.73 |
| ATOM | 1990 | CG1 | ILE | A | 251 | −1.670 | 67.562 | −0.169 | 1.00 | 18.11 |
| ATOM | 1991 | CD1 | ILE | A | 251 | −1.594 | 68.929 | −0.792 | 1.00 | 15.82 |
| ATOM | 1992 | C | ILE | A | 251 | 1.376 | 66.775 | 2.071 | 1.00 | 25.96 |
| ATOM | 1993 | O | ILE | A | 251 | 1.478 | 65.545 | 2.148 | 1.00 | 22.81 |
| ATOM | 1994 | N | ASN | A | 252 | 1.919 | 67.590 | 2.979 | 1.00 | 27.93 |
| ATOM | 1995 | CA | ASN | A | 252 | 2.583 | 67.097 | 4.190 | 1.00 | 23.28 |
| ATOM | 1996 | CB | ASN | A | 252 | 4.067 | 66.731 | 3.969 | 1.00 | 19.24 |
| ATOM | 1997 | CG | ASN | A | 252 | 4.949 | 67.922 | 3.669 | 1.00 | 15.95 |
| ATOM | 1998 | OD1 | ASN | A | 252 | 5.210 | 68.225 | 2.521 | 1.00 | 27.71 |
| ATOM | 1999 | ND2 | ASN | A | 252 | 5.482 | 68.544 | 4.698 | 1.00 | 12.20 |
| ATOM | 2000 | C | ASN | A | 252 | 2.417 | 68.107 | 5.321 | 1.00 | 26.77 |
| ATOM | 2001 | O | ASN | A | 252 | 2.046 | 69.258 | 5.079 | 1.00 | 26.57 |
| ATOM | 2002 | N | CYS | A | 253 | 2.621 | 67.649 | 6.555 | 1.00 | 26.39 |
| ATOM | 2003 | CA | CYS | A | 253 | 2.484 | 68.488 | 7.744 | 1.00 | 23.80 |
| ATOM | 2004 | CB | CYS | A | 253 | 2.069 | 67.638 | 8.950 | 1.00 | 26.15 |
| ATOM | 2005 | SG | CYS | A | 253 | 0.326 | 67.176 | 9.038 | 1.00 | 32.10 |
| ATOM | 2006 | C | CYS | A | 253 | 3.758 | 69.222 | 8.107 | 1.00 | 20.68 |
| ATOM | 2007 | O | CYS | A | 253 | 4.853 | 68.766 | 7.810 | 1.00 | 23.52 |
| ATOM | 2008 | N | GLY | A | 254 | 3.601 | 70.371 | 8.740 | 1.00 | 21.10 |
| ATOM | 2009 | CA | GLY | A | 254 | 4.740 | 71.143 | 9.183 | 1.00 | 21.44 |
| ATOM | 2010 | C | GLY | A | 254 | 4.870 | 70.886 | 10.669 | 1.00 | 19.86 |
| ATOM | 2011 | O | GLY | A | 254 | 4.062 | 70.173 | 11.245 | 1.00 | 17.78 |
| ATOM | 2012 | N | SER | A | 255 | 5.839 | 71.492 | 11.325 | 1.00 | 21.03 |
| ATOM | 2013 | CA | SER | A | 255 | 5.996 | 71.237 | 12.737 | 1.00 | 20.86 |
| ATOM | 2014 | CB | SER | A | 255 | 7.348 | 71.742 | 13.225 | 1.00 | 16.65 |
| ATOM | 2015 | OG | SER | A | 255 | 7.529 | 73.096 | 12.870 | 1.00 | 24.84 |
| ATOM | 2016 | C | SER | A | 255 | 4.862 | 71.800 | 13.592 | 1.00 | 25.80 |
| ATOM | 2017 | O | SER | A | 255 | 4.635 | 71.316 | 14.702 | 1.00 | 32.81 |
| ATOM | 2018 | N | TYR | A | 256 | 4.132 | 72.800 | 13.103 | 1.00 | 23.31 |
| ATOM | 2019 | CA | TYR | A | 256 | 3.048 | 73.337 | 13.916 | 1.00 | 22.05 |
| ATOM | 2020 | CB | TYR | A | 256 | 2.453 | 74.597 | 13.320 | 1.00 | 18.59 |
| ATOM | 2021 | CG | TYR | A | 256 | 1.600 | 75.359 | 14.313 | 1.00 | 18.81 |
| ATOM | 2022 | CD1 | TYR | A | 256 | 2.181 | 76.147 | 15.301 | 1.00 | 14.81 |
| ATOM | 2023 | CE1 | TYR | A | 256 | 1.399 | 76.878 | 16.198 | 1.00 | 18.34 |
| ATOM | 2024 | CD2 | TYR | A | 256 | 0.213 | 75.302 | 14.253 | 1.00 | 22.82 |
| ATOM | 2025 | CE2 | TYR | A | 256 | −0.586 | 76.029 | 15.151 | 1.00 | 22.41 |
| ATOM | 2026 | CZ | TYR | A | 256 | 0.015 | 76.812 | 16.122 | 1.00 | 22.48 |
| ATOM | 2027 | OH | TYR | A | 256 | −0.777 | 77.531 | 16.994 | 1.00 | 27.35 |
| ATOM | 2028 | C | TYR | A | 256 | 1.946 | 72.303 | 14.160 | 1.00 | 25.24 |
| ATOM | 2029 | O | TYR | A | 256 | 1.377 | 72.250 | 15.249 | 1.00 | 30.73 |
| ATOM | 2030 | N | MET | A | 257 | 1.645 | 71.490 | 13.152 | 1.00 | 25.21 |
| ATOM | 2031 | CA | MET | A | 257 | 0.633 | 70.443 | 13.281 | 1.00 | 25.59 |
| ATOM | 2032 | CB | MET | A | 257 | 0.422 | 69.749 | 11.930 | 1.00 | 23.11 |
| ATOM | 2033 | CG | MET | A | 257 | −0.631 | 68.646 | 11.889 | 1.00 | 23.77 |
| ATOM | 2034 | SD | MET | A | 257 | −2.303 | 69.226 | 12.196 | 1.00 | 29.13 |
| ATOM | 2035 | CE | MET | A | 257 | −2.926 | 69.464 | 10.571 | 1.00 | 21.14 |
| ATOM | 2036 | C | MET | A | 257 | 1.118 | 69.444 | 14.338 | 1.00 | 29.65 |
| ATOM | 2037 | O | MET | A | 257 | 0.348 | 68.996 | 15.182 | 1.00 | 33.68 |
| ATOM | 2038 | N | ALA | A | 258 | 2.410 | 69.139 | 14.324 | 1.00 | 30.20 |
| ATOM | 2039 | CA | ALA | A | 258 | 2.975 | 68.207 | 15.294 | 1.00 | 29.21 |
| ATOM | 2040 | CB | ALA | A | 258 | 4.437 | 67.918 | 14.962 | 1.00 | 26.53 |
| ATOM | 2041 | C | ALA | A | 258 | 2.843 | 68.740 | 16.721 | 1.00 | 29.82 |
| ATOM | 2042 | O | ALA | A | 258 | 2.486 | 67.999 | 17.635 | 1.00 | 30.07 |
| ATOM | 2043 | N | HIS | A | 259 | 3.130 | 70.023 | 16.912 | 1.00 | 29.81 |
| ATOM | 2044 | CA | HIS | A | 259 | 3.023 | 70.635 | 18.231 | 1.00 | 30.03 |
| ATOM | 2045 | CB | HIS | A | 259 | 3.560 | 72.061 | 18.197 | 1.00 | 29.44 |
| ATOM | 2046 | CG | HIS | A | 259 | 3.279 | 72.846 | 19.441 | 1.00 | 36.67 |
| ATOM | 2047 | CD2 | HIS | A | 259 | 3.973 | 72.971 | 20.600 | 1.00 | 35.37 |
| ATOM | 2048 | ND1 | HIS | A | 259 | 2.174 | 73.662 | 19.572 | 1.00 | 39.99 |
| ATOM | 2049 | CE1 | HIS | A | 259 | 2.201 | 74.255 | 20.750 | 1.00 | 40.19 |
| ATOM | 2050 | NE2 | HIS | A | 259 | 3.284 | 73.854 | 21.397 | 1.00 | 33.91 |
| ATOM | 2051 | C | HIS | A | 259 | 1.571 | 70.645 | 18.703 | 1.00 | 32.65 |
| ATOM | 2052 | O | HIS | A | 259 | 1.295 | 70.499 | 19.884 | 1.00 | 37.28 |
| ATOM | 2053 | N | LEU | A | 260 | 0.650 | 70.862 | 17.778 | 1.00 | 33.08 |
| ATOM | 2054 | CA | LEU | A | 260 | −0.770 | 70.900 | 18.092 | 1.00 | 31.90 |
| ATOM | 2055 | CB | LEU | A | 260 | −1.543 | 71.402 | 16.880 | 1.00 | 30.79 |
| ATOM | 2056 | CG | LEU | A | 260 | −2.224 | 72.751 | 16.957 | 1.00 | 33.95 |
| ATOM | 2057 | CD1 | LEU | A | 260 | −1.342 | 73.763 | 17.664 | 1.00 | 35.15 |
| ATOM | 2058 | CD2 | LEU | A | 260 | −2.549 | 73.184 | 15.536 | 1.00 | 39.19 |
| ATOM | 2059 | C | LEU | A | 260 | −1.326 | 69.536 | 18.470 | 1.00 | 33.38 |
| ATOM | 2060 | O | LEU | A | 260 | −2.082 | 69.411 | 19.420 | 1.00 | 35.86 |
| ATOM | 2061 | N | THR | A | 261 | −0.988 | 68.526 | 17.684 | 1.00 | 32.53 |
| ATOM | 2062 | CA | THR | A | 261 | −1.480 | 67.184 | 17.905 | 1.00 | 33.81 |
| ATOM | 2063 | CB | THR | A | 261 | −1.571 | 66.443 | 16.580 | 1.00 | 35.83 |
| ATOM | 2064 | OG1 | THR | A | 261 | −0.270 | 66.392 | 15.977 | 1.00 | 33.48 |
| ATOM | 2065 | CG2 | THR | A | 261 | −2.537 | 67.155 | 15.647 | 1.00 | 37.64 |
| ATOM | 2066 | C | THR | A | 261 | −0.590 | 66.389 | 18.840 | 1.00 | 39.46 |
| ATOM | 2067 | O | THR | A | 261 | −0.651 | 65.153 | 18.870 | 1.00 | 38.94 |
| ATOM | 2068 | N | ASN | A | 262 | 0.267 | 67.094 | 19.572 | 1.00 | 44.19 |
| ATOM | 2069 | CA | ASN | A | 262 | 1.191 | 66.456 | 20.506 | 1.00 | 49.58 |
| ATOM | 2070 | CB | ASN | A | 262 | 0.445 | 65.952 | 21.756 | 1.00 | 59.13 |
| ATOM | 2071 | CG | ASN | A | 262 | 1.353 | 65.841 | 22.981 | 1.00 | 66.86 |
| ATOM | 2072 | OD1 | ASN | A | 262 | 1.367 | 66.737 | 23.833 | 1.00 | 72.04 |
| ATOM | 2073 | ND2 | ASN | A | 262 | 2.105 | 64.743 | 23.081 | 1.00 | 67.84 |
| ATOM | 2074 | C | ASN | A | 262 | 1.941 | 65.307 | 19.811 | 1.00 | 46.90 |
| ATOM | 2075 | O | ASN | A | 262 | 2.228 | 64.274 | 20.415 | 1.00 | 49.43 |
| ATOM | 2076 | N | ASN | A | 263 | 2.208 | 65.492 | 18.522 | 1.00 | 41.44 |
| ATOM | 2077 | CA | ASN | A | 263 | 2.929 | 64.534 | 17.698 | 1.00 | 37.52 |
| ATOM | 2078 | C13 | ASN | A | 263 | 4.237 | 64.119 | 18.347 | 1.00 | 41.11 |
| ATOM | 2079 | CG | ASN | A | 263 | 5.415 | 64.740 | 17.670 | 1.00 | 47.69 |
| ATOM | 2080 | OD1 | ASN | A | 263 | 5.928 | 65.764 | 18.109 | 1.00 | 48.68 |
| ATOM | 2081 | ND2 | ASN | A | 263 | 5.824 | 64.155 | 16.550 | 1.00 | 54.33 |
| ATOM | 2082 | C | ASN | A | 263 | 2.201 | 63.322 | 17.172 | 1.00 | 35.18 |
| ATOM | 2083 | O | ASN | A | 263 | 2.832 | 62.388 | 16.679 | 1.00 | 34.27 |
| ATOM | 2084 | N | TYR | A | 264 | 0.877 | 63.344 | 17.250 | 1.00 | 36.95 |
| ATOM | 2085 | CA | TYR | A | 264 | 0.063 | 62.252 | 16.723 | 1.00 | 37.39 |
| TOM | 2086 | CB | TYR | A | 264 | −1.393 | 62.413 | 17.189 | 1.00 | 33.82 |
| ATOM | 2087 | CG | TYR | A | 264 | −2.344 | 61.342 | 16.713 | 1.00 | 33.63 |
| ATOM | 2088 | CD1 | TYR | A | 264 | −2.446 | 60.113 | 17.362 | 1.00 | 32.49 |
| ATOM | 2089 | CE1 | TYR | A | 264 | −3.375 | 59.151 | 16.935 | 1.00 | 33.06 |
| ATOM | 2090 | CD2 | TYR | A | 264 | −3.180 | 61.579 | 15.627 | 1.00 | 37.61 |
| ATOM | 2091 | CE2 | TYR | A | 264 | −4.105 | 60.636 | 15.195 | 1.00 | 37.45 |
| ATOM | 2092 | CZ | TYR | A | 264 | −4.204 | 59.429 | 15.845 | 1.00 | 35.62 |
| ATOM | 2093 | OH | TYR | A | 264 | −5.169 | 58.546 | 15.403 | 1.00 | 38.87 |
| ATOM | 2094 | C | TYR | A | 264 | 0.218 | 62.311 | 15.186 | 1.00 | 37.01 |
| ATOM | 2095 | O | TYR | A | 264 | 0.169 | 61.287 | 14.499 | 1.00 | 37.37 |
| ATOM | 2096 | N | TYR | A | 265 | 0.390 | 63.528 | 14.666 | 1.00 | 36.23 |
| ATOM | 2097 | CA | TYR | A | 265 | 0.642 | 63.768 | 13.244 | 1.00 | 33.20 |
| ATOM | 2098 | CB | TYR | A | 265 | −0.351 | 64.750 | 12.640 | 1.00 | 26.57 |
| ATOM | 2099 | CG | TYR | A | 265 | −1.642 | 64.115 | 12.239 | 1.00 | 31.72 |
| ATOM | 2100 | CD1 | TYR | A | 265 | −2.630 | 64.861 | 11.610 | 1.00 | 33.04 |
| ATOM | 2101 | CE1 | TYR | A | 265 | −3.854 | 64.298 | 11.286 | 1.00 | 30.94 |
| ATOM | 2102 | CD2 | TYR | A | 265 | −1.909 | 62.775 | 12.527 | 1.00 | 32.98 |
| ATOM | 2103 | CE2 | TYR | A | 265 | −3.141 | 62.201 | 12.207 | 1.00 | 31.76 |
| ATOM | 2104 | CZ | TYR | A | 265 | −4.102 | 62.976 | 11.591 | 1.00 | 30.37 |
| ATOM | 2105 | OH | TYR | A | 265 | −5.333 | 62.452 | 11.312 | 1.00 | 38.15 |
| ATOM | 2106 | C | TYR | A | 265 | 2.028 | 64.390 | 13.227 | 1.00 | 34.58 |
| ATOM | 2107 | O | TYR | A | 265 | 2.187 | 65.586 | 13.466 | 1.00 | 35.29 |
| ATOM | 2108 | N | LYS | A | 266 | 3.036 | 63.553 | 13.022 | 1.00 | 34.31 |
| ATOM | 2109 | CA | LYS | A | 266 | 4.422 | 63.990 | 13.002 | 1.00 | 33.62 |
| ATOM | 2110 | CB | LYS | A | 266 | 5.328 | 62.772 | 13.039 | 1.00 | 39.20 |
| ATOM | 2111 | CG | LYS | A | 266 | 6.739 | 63.066 | 13.491 | 1.00 | 56.22 |
| ATOM | 2112 | CD | LYS | A | 266 | 7.549 | 61.773 | 13.584 | 1.00 | 67.11 |
| ATOM | 2113 | CE | LYS | A | 266 | 6.797 | 60.697 | 14.372 | 1.00 | 74.71 |
| ATOM | 2114 | NZ | LYS | A | 266 | 6.402 | 61.147 | 15.748 | 1.00 | 80.37 |
| ATOM | 2115 | C | LYS | A | 266 | 4.748 | 64.784 | 11.759 | 1.00 | 29.29 |
| ATOM | 2116 | O | LYS | A | 266 | 4.184 | 64.540 | 10.702 | 1.00 | 30.14 |
| ATOM | 2117 | N | ALA | A | 267 | 5.641 | 65.752 | 11.890 | 1.00 | 26.90 |
| ATOM | 2118 | CA | ALA | A | 267 | 6.063 | 66.526 | 10.735 | 1.00 | 27.00 |
| ATOM | 2119 | CB | ALA | A | 267 | 6.724 | 67.809 | 11.173 | 1.00 | 22.67 |
| ATOM | 2120 | C | ALA | A | 267 | 7.079 | 65.599 | 10.072 | 1.00 | 28.13 |
| ATOM | 2121 | O | ALA | A | 267 | 8.074 | 65.243 | 10.695 | 1.00 | 31.38 |
| ATOM | 2122 | N | PRO | A | 268 | 6.822 | 65.155 | 8.823 | 1.00 | 30.02 |
| ATOM | 2123 | CD | PRO | A | 268 | 5.648 | 65.466 | 7.989 | 1.00 | 29.53 |
| ATOM | 2124 | Q | PRO | A | 268 | 7.732 | 64.251 | 8.103 | 1.00 | 28.12 |
| ATOM | 2125 | CB | PRO | A | 268 | 7.043 | 64.081 | 6.742 | 1.00 | 25.57 |
| ATOM | 2126 | CG | PRO | A | 268 | 5.615 | 64.286 | 7.035 | 1.00 | 25.50 |
| ATOM | 2127 | C | PRO | A | 268 | 9.133 | 64.805 | 7.904 | 1.00 | 27.55 |
| ATOM | 2128 | O | PRO | A | 268 | 9.281 | 65.974 | 7.555 | 1.00 | 31.71 |
| ATOM | 2129 | N | ILE | A | 269 | 10.153 | 63.984 | 8.156 | 1.00 | 26.79 |
| ATOM | 2130 | Q | ILE | A | 269 | 11.549 | 64.374 | 7.939 | 1.00 | 21.70 |
| ATOM | 2131 | CB | ILE | A | 269 | 12.527 | 63.535 | 8.799 | 1.00 | 23.68 |
| ATOM | 2132 | CG2 | ILE | A | 269 | 13.971 | 63.768 | 8.358 | 1.00 | 16.90 |
| ATOM | 2133 | CG1 | ILE | A | 269 | 12.371 | 63.881 | 10.286 | 1.00 | 25.36 |
| ATOM | 2134 | CD1 | ILE | A | 269 | 12.761 | 65.311 | 10.645 | 1.00 | 21.95 |
| ATOM | 2135 | C | ILE | A | 269 | 11.788 | 64.052 | 6.470 | 1.00 | 21.77 |
| ATOM | 2136 | O | HIS | A | 269 | 11.544 | 62.932 | 6.025 | 1.00 | 23.31 |
| ATOM | 2137 | N | HIS | A | 270 | 12.196 | 65.043 | 5.696 | 1.00 | 22.73 |
| ATOM | 2138 | CA | HIS | A | 270 | 12.420 | 64.808 | 4.280 | 1.00 | 21.55 |
| ATOM | 2139 | CB | HIS | A | 270 | 11.161 | 65.146 | 3.480 | 1.00 | 20.47 |
| ATOM | 2140 | CG | HIS | A | 270 | 10.613 | 66.507 | 3.758 | 1.00 | 16.63 |
| ATOM | 2141 | CD2 | HIS | A | 270 | 10.417 | 67.570 | 2.946 | 1.00 | 20.91 |
| ATOM | 2142 | ND1 | HIS | A | 270 | 10.148 | 66.885 | 4.997 | 1.00 | 18.74 |
| ATOM | 2143 | CE1 | HIS | A | 270 | 9.680 | 68.117 | 4.936 | 1.00 | 20.88 |
| ATOM | 2144 | NE2 | HIS | A | 270 | 9.828 | 68.559 | 3.700 | 1.00 | 17.58 |
| ATOM | 2145 | C | HIS | A | 270 | 13.608 | 65.600 | 3.783 | 1.00 | 22.67 |
| ATOM | 2146 | O | HIS | A | 270 | 14.008 | 66.588 | 4.410 | 1.00 | 23.83 |
| ATOM | 2147 | N | ARG | A | 271 | 14.201 | 65.136 | 2.689 | 1.00 | 20.85 |
| ATOM | 2148 | CA | ARG | A | 271 | 15.359 | 65.797 | 2.093 | 1.00 | 23.79 |
| ATOM | 2149 | CB | ARG | A | 271 | 16.631 | 65.043 | 2.461 | 1.00 | 22.83 |
| ATOM | 2150 | CG | ARG | A | 271 | 16.615 | 63.592 | 2.057 | 1.00 | 23.45 |
| ATOM | 2151 | CD | ARG | A | 271 | 17.872 | 62.894 | 2.523 | 1.00 | 25.22 |
| ATOM | 2152 | NE | ARG | A | 271 | 18.020 | 61.603 | 1.862 | 1.00 | 26.89 |
| ATOM | 2153 | CZ | ARG | A | 271 | 19.008 | 60.753 | 2.103 | 1.00 | 25.53 |
| ATOM | 2154 | NH1 | ARG | A | 271 | 19.931 | 61.058 | 2.996 | 1.00 | 29.48 |
| ATOM | 2155 | NH2 | ARG | A | 271 | 19.081 | 59.607 | 1.441 | 1.00 | 25.73 |
| ATOM | 2156 | C | ARG | A | 271 | 15.190 | 65.873 | 0.580 | 1.00 | 23.68 |
| ATOM | 2157 | O | ARG | A | 271 | 14.319 | 65.211 | 0.010 | 1.00 | 26.81 |
| ATOM | 2158 | N | VAL | A | 272 | 15.994 | 66.702 | −0.071 | 1.00 | 22.19 |
| ATOM | 2159 | Q | VAL | A | 272 | 15.878 | 66.857 | −1.510 | 1.00 | 18.76 |
| ATOM | 2160 | CB | VAL | A | 272 | 15.561 | 68.310 | −1.870 | 1.00 | 17.63 |
| ATOM | 2161 | CG1 | VAL | A | 272 | 15.326 | 68.446 | −3.355 | 1.00 | 18.50 |
| ATOM | 2162 | CG2 | VAL | A | 272 | 14.340 | 68.778 | −1.107 | 1.00 | 15.11 |
| ATOM | 2163 | C | VAL | A | 272 | 17.132 | 66.418 | −2.248 | 1.00 | 25.28 |
| ATOM | 2164 | O | VAL | A | 272 | 18.202 | 66.993 | −2.050 | 1.00 | 29.40 |
| ATOM | 2165 | N | LYS | A | 273 | 16.985 | 65.396 | −3.092 | 1.00 | 27.72 |
| ATOM | 2166 | CA | LYS | A | 273 | 18.059 | 64.845 | −3.919 | 1.00 | 25.89 |
| ATOM | 2167 | CB | LYS | A | 273 | 17.572 | 63.587 | −4.645 | 1.00 | 27.95 |
| ATOM | 2168 | CG | LYS | A | 273 | 17.355 | 62.392 | −3.753 | 1.00 | 31.66 |
| ATOM | 2169 | CD | LYS | A | 273 | 16.404 | 61.385 | −4.376 | 1.00 | 35.66 |
| ATOM | 2170 | CE | LYS | A | 273 | 16.963 | 60.740 | −5.621 | 1.00 | 41.21 |
| ATOM | 2171 | NZ | LYS | A | 273 | 16.185 | 59.514 | −5.974 | 1.00 | 42.41 |
| ATOM | 2172 | C | LYS | A | 273 | 18.551 | 65.837 | −4.968 | 1.00 | 25.96 |
| ATOM | 2173 | O | LYS | A | 273 | 17.784 | 66.653 | −5.486 | 1.00 | 26.05 |
| ATOM | 2174 | N | TRP | A | 274 | 19.843 | 65.759 | −5.268 | 1.00 | 25.99 |
| ATOM | 2175 | Q | TRP | A | 274 | 20.459 | 66.616 | −6.267 | 1.00 | 24.01 |
| ATOM | 2176 | CB | TRP | A | 274 | 21.975 | 66.623 | −6.096 | 1.00 | 24.20 |
| ATOM | 2177 | CG | TRP | A | 274 | 22.700 | 67.347 | −7.187 | 1.00 | 23.66 |
| ATOM | 2178 | CD2 | TRP | A | 274 | 23.526 | 66.762 | −8.194 | 1.00 | 21.71 |
| ATOM | 2179 | CE2 | TRP | A | 274 | 23.997 | 67.812 | −9.008 | 1.00 | 21.28 |
| ATOM | 2180 | CE3 | TRP | A | 274 | 23.900 | 65.453 | −8.498 | 1.00 | 25.08 |
| ATOM | 2181 | CD1 | TRP | A | 274 | 22.709 | 68.689 | −7.417 | 1.00 | 26.21 |
| ATOM | 2182 | NE1 | TRP | A | 274 | 23.490 | 68.979 | −8.509 | 1.00 | 28.27 |
| ATOM | 2183 | CZ2 | TRP | A | 274 | 24.839 | 67.594 | −10.089 | 1.00 | 23.56 |
| ATOM | 2184 | CZ3 | TRP | A | 274 | 24.739 | 65.237 | −9.582 | 1.00 | 29.23 |
| ATOM | 2185 | CH2 | TRP | A | 274 | 25.193 | 66.305 | −10.367 | 1.00 | 24.09 |
| ATOM | 2186 | C | TRP | A | 274 | 20.105 | 66.099 | −7.653 | 1.00 | 27.26 |
| ATOM | 2187 | O | TRP | A | 274 | 20.357 | 64.938 | −7.970 | 1.00 | 32.63 |
| ATOM | 2188 | N | VAL | A | 275 | 19.503 | 66.964 | −8.461 | 1.00 | 28.27 |
| ATOM | 2189 | CA | VAL | A | 275 | 19.110 | 66.636 | −9.825 | 1.00 | 25.56 |
| ATOM | 2190 | CB | VAL | A | 275 | 17.582 | 66.606 | −9.971 | 1.00 | 23.27 |
| ATOM | 2191 | CG1 | VAL | A | 275 | 17.200 | 66.358 | −11.410 | 1.00 | 23.05 |
| ATOM | 2192 | CG2 | VAL | A | 275 | 16.993 | 65.545 | −9.073 | 1.00 | 25.58 |
| ATOM | 2193 | C | VAL | A | 275 | 19.645 | 67.760 | −10.689 | 1.00 | 23.04 |
| ATOM | 2194 | O | VAL | A | 275 | 19.194 | 68.884 | −10.573 | 1.00 | 27.65 |
| ATOM | 2195 | N | ASN | A | 276 | 20.627 | 67.480 | −11.532 | 1.00 | 24.79 |
| ATOM | 2196 | CA | ASN | A | 276 | 21.172 | 68.539 | −12.372 | 1.00 | 23.43 |
| ATOM | 2197 | CB | ASN | A | 276 | 22.626 | 68.262 | −12.737 | 1.00 | 23.64 |
| ATOM | 2198 | CG | ASN | A | 276 | 23.237 | 69.374 | −13.563 | 1.00 | 27.31 |
| ATOM | 2199 | OD1 | ASN | A | 276 | 22.697 | 70.479 | −13.650 | 1.00 | 25.48 |
| ATOM | 2200 | ND2 | ASN | A | 276 | 24.382 | 69.096 | −14.156 | 1.00 | 32.14 |
| ATOM | 2201 | C | ASN | A | 276 | 20.347 | 68.732 | −13.632 | 1.00 | 24.63 |
| ATOM | 2202 | O | ASN | A | 276 | 20.683 | 68.215 | −14.694 | 1.00 | 31.02 |
| ATOM | 2203 | N | ALA | A | 277 | 19.244 | 69.453 | −13.499 | 1.00 | 25.78 |
| ATOM | 2204 | CA | ALA | A | 277 | 18.364 | 69.719 | −14.620 | 1.00 | 23.03 |
| ATOM | 2205 | CB | ALA | A | 277 | 17.502 | 68.507 | −14.911 | 1.00 | 26.09 |
| ATOM | 2206 | C | ALA | A | 277 | 17.490 | 70.903 | −14.288 | 1.00 | 25.78 |
| ATOM | 2207 | O | ALA | A | 277 | 17.232 | 71.177 | −13.119 | 1.00 | 27.01 |
| ATOM | 2208 | N | GLU | A | 278 | 17.034 | 71.602 | −15.321 | 1.00 | 25.92 |
| ATOM | 2209 | CA | GLU | A | 278 | 16.176 | 72.760 | −15.139 | 1.00 | 27.97 |
| ATOM | 2210 | CB | GLU | A | 278 | 16.111 | 73.616 | −16.399 | 1.00 | 29.38 |
| ATOM | 2211 | CG | GLU | A | 278 | 17.326 | 74.473 | −16.634 | 1.00 | 34.40 |
| ATOM | 2212 | CD | GLU | A | 278 | 17.373 | 75.697 | −15.764 | 1.00 | 31.39 |
| ATOM | 2213 | OE1 | GLU | A | 278 | 16.312 | 76.182 | −15.327 | 1.00 | 35.93 |
| ATOM | 2214 | OE2 | GLU | A | 278 | 18.489 | 76.186 | −15.537 | 1.00 | 37.93 |
| ATOM | 2215 | C | GLU | A | 278 | 14.777 | 72.323 | −14.767 | 1.00 | 30.01 |
| ATOM | 2216 | O | GLU | A | 278 | 13.999 | 71.867 | −15.601 | 1.00 | 33.28 |
| ATOM | 2217 | N | ARG | A | 279 | 14.485 | 72.419 | −13.487 | 1.00 | 30.78 |
| ATOM | 2218 | CA | ARG | A | 279 | 13.185 | 72.069 | −12.986 | 1.00 | 29.39 |
| ATOM | 2219 | CB | ARG | A | 279 | 13.232 | 70.718 | −12.294 | 1.00 | 29.88 |
| ATOM | 2220 | CG | ARG | A | 279 | 13.756 | 69.606 | −13.168 | 1.00 | 29.15 |
| ATOM | 2221 | CD | ARG | A | 279 | 12.891 | 69.382 | −14.381 | 1.00 | 26.70 |
| ATOM | 2222 | NE | ARG | A | 279 | 13.340 | 68.183 | −15.073 | 1.00 | 28.42 |
| ATOM | 2223 | CZ | ARG | A | 279 | 13.978 | 68.185 | −16.234 | 1.00 | 25.05 |
| ATOM | 2224 | NH1 | ARG | A | 279 | 14.224 | 69.324 | −16.855 | 1.00 | 21.11 |
| ATOM | 2225 | NH2 | ARG | A | 279 | 14.477 | 67.059 | −16.716 | 1.00 | 26.84 |
| ATOM | 2226 | C | ARG | A | 279 | 12.813 | 73.163 | −12.007 | 1.00 | 30.45 |
| ATOM | 2227 | O | ARG | A | 279 | 13.645 | 74.001 | −11.643 | 1.00 | 28.64 |
| ATOM | 2228 | N | GLN | A | 280 | 11.560 | 73.147 | −11.583 | 1.00 | 31.12 |
| ATOM | 2229 | CA | GLN | A | 280 | 11.045 | 74.134 | −10.661 | 1.00 | 33.15 |
| ATOM | 2230 | CB | GLN | A | 280 | 10.260 | 75.182 | −11.427 | 1.00 | 38.18 |
| ATOM | 2231 | CG | GLN | A | 280 | 9.171 | 74.580 | −12.286 | 1.00 | 53.46 |
| ATOM | 2232 | CD | GLN | A | 280 | 8.596 | 75.565 | −13.278 | 1.00 | 57.41 |
| ATOM | 2233 | OE1 | GLN | A | 280 | 9.209 | 76.593 | −13.568 | 1.00 | 60.52 |
| ATOM | 2234 | NE2 | GLN | A | 280 | 7.418 | 75.250 | −13.820 | 1.00 | 60.10 |
| ATOM | 2235 | C | GLN | A | 280 | 10.138 | 73.430 | −9.679 | 1.00 | 30.33 |
| ATOM | 2236 | O | GLN | A | 280 | 9.602 | 72.363 | −9.975 | 1.00 | 32.02 |
| ATOM | 2237 | N | SER | A | 281 | 9.958 | 74.049 | −8.521 | 1.00 | 26.28 |
| ATOM | 2238 | CA | SER | A | 281 | 9.131 | 73.510 | −7.462 | 1.00 | 18.83 |
| ATOM | 2239 | CB | SER | A | 281 | 10.034 | 72.908 | −6.390 | 1.00 | 16.21 |
| ATOM | 2240 | OG | SER | A | 281 | 9.281 | 72.175 | −5.458 | 1.00 | 31.28 |
| ATOM | 2241 | C | SER | A | 281 | 8.344 | 74.691 | −6.918 | 1.00 | 17.91 |
| ATOM | 2242 | O | SER | A | 281 | 8.870 | 75.796 | −6.852 | 1.00 | 15.21 |
| ATOM | 2243 | N | LEU | A | 282 | 7.070 | 74.487 | −6.596 | 1.00 | 24.14 |
| ATOM | 2244 | CA | LEU | A | 282 | 6.239 | 75.578 | −6.068 | 1.00 | 24.06 |
| ATOM | 2245 | CB | LEU | A | 282 | 5.224 | 76.049 | −7.098 | 1.00 | 26.03 |
| ATOM | 2246 | CG | LEU | A | 282 | 5.733 | 76.662 | −8.388 | 1.00 | 34.69 |
| ATOM | 2247 | CD1 | LEU | A | 282 | 6.212 | 75.564 | −9.339 | 1.00 | 37.28 |
| ATOM | 2248 | CD2 | LEU | A | 282 | 4.582 | 77.421 | −9.009 | 1.00 | 38.00 |
| ATOM | 2249 | C | LEU | A | 282 | 5.480 | 75.182 | −4.814 | 1.00 | 23.88 |
| ATOM | 2250 | O | LEU | A | 282 | 4.368 | 74.652 | −4.894 | 1.00 | 21.16 |
| ATOM | 2251 | N | PRO | A | 283 | 6.109 | 75.372 | −3.637 | 1.00 | 22.81 |
| ATOM | 2252 | CD | PRO | A | 283 | 7.531 | 75.682 | −3.434 | 1.00 | 22.38 |
| ATOM | 2253 | CA | PRO | A | 283 | 5.463 | 75.035 | −2.372 | 1.00 | 20.82 |
| ATOM | 2254 | CB | PRO | A | 283 | 6.661 | 74.739 | −1.472 | 1.00 | 18.71 |
| ATOM | 2255 | CG | PRO | A | 283 | 7.663 | 75.746 | −1.928 | 1.00 | 19.17 |
| ATOM | 2256 | C | PRO | A | 283 | 4.651 | 76.192 | −1.847 | 1.00 | 17.56 |
| ATOM | 2257 | O | PRO | A | 283 | 5.028 | 77.349 | −2.001 | 1.00 | 19.99 |
| ATOM | 2258 | N | PHE | A | 284 | 3.496 | 75.874 | −1.282 | 1.00 | 18.04 |
| ATOM | 2259 | CA | PHE | A | 284 | 2.628 | 76.869 | −0.686 | 1.00 | 14.83 |
| ATOM | 2260 | CB | PHE | A | 284 | 1.202 | 76.739 | −1.221 | 1.00 | 13.11 |
| ATOM | 2261 | CG | PHE | A | 284 | 0.222 | 77.695 | −0.590 | 1.00 | 9.51 |
| ATOM | 2262 | CD1 | PHE | A | 284 | 0.361 | 79.072 | −0.757 | 1.00 | 7.91 |
| ATOM | 2263 | CD2 | PHE | A | 284 | −0.840 | 77.218 | 0.176 | 1.00 | 8.25 |
| ATOM | 2264 | CE1 | PHE | A | 284 | −0.546 | 79.963 | −0.172 | 1.00 | 9.04 |
| ATOM | 2265 | CE2 | PHE | A | 284 | −1.755 | 78.103 | 0.769 | 1.00 | 7.90 |
| ATOM | 2266 | CZ | PHE | A | 284 | −1.607 | 79.480 | 0.596 | 1.00 | 7.48 |
| ATOM | 2267 | C | PHE | A | 284 | 2.657 | 76.481 | 0.777 | 1.00 | 15.38 |
| ATOM | 2268 | O | PHE | A | 284 | 2.315 | 75.351 | 1.126 | 1.00 | 13.28 |
| ATOM | 2269 | N | PHE | A | 285 | 3.130 | 77.381 | 1.625 | 1.00 | 11.26 |
| ATOM | 2270 | CA | PHE | A | 285 | 3.204 | 77.096 | 3.045 | 1.00 | 11.08 |
| ATOM | 2271 | CB | PHE | A | 285 | 4.488 | 77.682 | 3.622 | 1.00 | 11.89 |
| ATOM | 2272 | CG | PHE | A | 285 | 5.734 | 77.103 | 3.021 | 1.00 | 13.37 |
| ATOM | 2273 | CD1 | PHE | A | 285 | 6.418 | 77.779 | 2.024 | 1.00 | 12.80 |
| ATOM | 2274 | CD2 | PHE | A | 285 | 6.229 | 75.882 | 3.458 | 1.00 | 14.08 |
| ATOM | 2275 | CE1 | PHE | A | 285 | 7.584 | 77.245 | 1.477 | 1.00 | 16.30 |
| ATOM | 2276 | CE2 | PHE | A | 285 | 7.396 | 75.347 | 2.911 | 1.00 | 13.47 |
| ATOM | 2277 | CZ | PHE | A | 285 | 8.066 | 76.030 | 1.925 | 1.00 | 7.59 |
| ATOM | 2278 | C | PHE | A | 285 | 1.985 | 77.644 | 3.756 | 1.00 | 12.95 |
| ATOM | 2279 | O | PHE | A | 285 | 1.781 | 78.863 | 3.801 | 1.00 | 14.08 |
| ATOM | 2280 | N | VAL | A | 286 | 1.150 | 76.746 | 4.271 | 1.00 | 11.34 |
| ATOM | 2281 | CA | VAL | A | 286 | −0.062 | 77.158 | 4.969 | 1.00 | 15.20 |
| ATOM | 2282 | CB | VAL | A | 286 | −1.128 | 76.021 | 4.997 | 1.00 | 13.61 |
| ATOM | 2283 | CG1 | VAL | A | 286 | −2.365 | 76.467 | 5.739 | 1.00 | 11.57 |
| ATOM | 2284 | CG2 | VAL | A | 286 | −1.511 | 75.629 | 3.587 | 1.00 | 10.57 |
| ATOM | 2285 | C | VAL | A | 286 | 0.271 | 77.647 | 6.384 | 1.00 | 18.76 |
| ATOM | 2286 | O | VAL | A | 286 | 0.667 | 76.876 | 7.257 | 1.00 | 24.10 |
| ATOM | 2287 | N | ASN | A | 287 | 0.190 | 78.955 | 6.571 | 1.00 | 17.87 |
| ATOM | 2288 | CA | ASN | A | 287 | 0.461 | 79.568 | 7.850 | 1.00 | 18.13 |
| ATOM | 2289 | CB | ASN | A | 287 | 1.570 | 80.621 | 7.722 | 1.00 | 19.63 |
| ATOM | 2290 | CG | ASN | A | 287 | 2.940 | 80.017 | 7.427 | 1.00 | 17.67 |
| ATOM | 2291 | OD1 | ASN | A | 287 | 3.287 | 78.940 | 7.907 | 1.00 | 15.97 |
| ATOM | 2292 | ND2 | ASN | A | 287 | 3.729 | 80.727 | 6.644 | 1.00 | 19.12 |
| ATOM | 2293 | C | ASN | A | 287 | −0.844 | 80.242 | 8.229 | 1.00 | 21.86 |
| ATOM | 2294 | O | ASN | A | 287 | −1.584 | 80.680 | 7.347 | 1.00 | 26.85 |
| ATOM | 2295 | N | LEU | A | 288 | −1.148 | 80.302 | 9.523 | 1.00 | 19.51 |
| ATOM | 2296 | CA | LEU | A | 288 | −2.374 | 80.928 | 9.979 | 1.00 | 14.83 |
| ATOM | 2297 | CB | LEU | A | 288 | −2.853 | 80.254 | 11.253 | 1.00 | 12.62 |
| ATOM | 2298 | CG | LEU | A | 288 | −2.982 | 78.741 | 11.075 | 1.00 | 17.68 |
| ATOM | 2299 | CD1 | LEU | A | 288 | −3.540 | 78.114 | 12.332 | 1.00 | 16.20 |
| ATOM | 2300 | CD2 | LEU | A | 288 | −3.883 | 78.428 | 9.909 | 1.00 | 14.63 |
| ATOM | 2301 | C | LEU | A | 288 | −2.139 | 82.414 | 10.188 | 1.00 | 16.91 |
| ATOM | 2302 | O | LEU | A | 288 | −1.218 | 82.981 | 9.611 | 1.00 | 20.51 |
| ATOM | 2303 | N | GLY | A | 289 | −2.974 | 83.051 | 10.996 | 1.00 | 16.67 |
| ATOM | 2304 | CA | GLY | A | 289 | −2.823 | 84.473 | 11.240 | 1.00 | 15.37 |
| ATOM | 2305 | C | GLY | A | 289 | −1.831 | 84.700 | 12.350 | 1.00 | 19.82 |
| ATOM | 2306 | O | GLY | A | 289 | −1.571 | 83.795 | 13.130 | 1.00 | 22.79 |
| ATOM | 2307 | N | TYR | A | 290 | −1.342 | 85.924 | 12.477 | 1.00 | 19.69 |
| ATOM | 2308 | CA | TYR | A | 290 | −0.354 | 86.246 | 13.491 | 1.00 | 23.55 |
| ATOM | 2309 | CB | TYR | A | 290 | 0.058 | 87.705 | 13.359 | 1.00 | 22.89 |
| ATOM | 2310 | CG | TYR | A | 290 | 1.265 | 88.073 | 14.184 | 1.00 | 30.95 |
| ATOM | 2311 | CD1 | TYR | A | 290 | 2.558 | 87.793 | 13.730 | 1.00 | 31.65 |
| ATOM | 2312 | CE1 | TYR | A | 290 | 3.671 | 88.142 | 14.481 | 1.00 | 32.74 |
| ATOM | 2313 | CD2 | TYR | A | 290 | 1.122 | 88.710 | 15.414 | 1.00 | 29.60 |
| ATOM | 2314 | CE2 | TYR | A | 290 | 2.228 | 89.063 | 16.172 | 1.00 | 31.69 |
| ATOM | 2315 | CZ | TYR | A | 290 | 3.494 | 88.778 | 15.699 | 1.00 | 32.60 |
| ATOM | 2316 | OH | TYR | A | 290 | 4.587 | 89.149 | 16.435 | 1.00 | 38.35 |
| ATOM | 2317 | C | TYR | A | 290 | −0.825 | 85.973 | 14.912 | 1.00 | 28.88 |
| ATOM | 2318 | O | TYR | A | 290 | −0.064 | 85.469 | 15.747 | 1.00 | 32.22 |
| ATOM | 2319 | N | ASP | A | 291 | −2.080 | 86.302 | 15.180 | 1.00 | 32.16 |
| ATOM | 2320 | CA | ASP | A | 291 | −2.650 | 86.121 | 16.505 | 1.00 | 34.89 |
| ATOM | 2321 | CB | ASP | A | 291 | −3.621 | 87.271 | 16.809 | 1.00 | 44.67 |
| ATOM | 2322 | CG | ASP | A | 291 | −2.907 | 88.607 | 17.064 | 1.00 | 54.47 |
| ATOM | 2323 | OD1 | ASP | A | 291 | −1.678 | 88.612 | 17.294 | 1.00 | 62.50 |
| ATOM | 2324 | OD2 | ASP | A | 291 | −3.583 | 89.662 | 17.057 | 1.00 | 59.68 |
| ATOM | 2325 | C | ASP | A | 291 | −3.341 | 84.786 | 16.743 | 1.00 | 33.13 |
| ATOM | 2326 | O | ASP | A | 291 | −3.867 | 84.552 | 17.828 | 1.00 | 36.71 |
| ATOM | 2327 | N | SER | A | 292 | −3.325 | 83.902 | 15.755 | 1.00 | 32.93 |
| ATOM | 2328 | CA | SER | A | 292 | −3.989 | 82.611 | 13.896 | 1.00 | 33.04 |
| ATOM | 2329 | CB | SER | A | 292 | −4.074 | 81.892 | 14.551 | 1.00 | 32.62 |
| ATOM | 2330 | OG | SER | A | 292 | −4.870 | 82.629 | 13.641 | 1.00 | 37.64 |
| ATOM | 2331 | C | SER | A | 292 | −3.394 | 81.675 | 16.933 | 1.00 | 33.50 |
| ATOM | 2332 | O | SER | A | 292 | −2.223 | 81.310 | 16.870 | 1.00 | 35.88 |
| ATOM | 2333 | N | VAL | A | 293 | −4.223 | 81.283 | 17.889 | 1.00 | 35.69 |
| ATOM | 2334 | CA | VAL | A | 293 | −3.807 | 80.371 | 18.936 | 1.00 | 33.43 |
| ATOM | 2335 | CB | VAL | A | 293 | −3.867 | 81.015 | 20.333 | 1.00 | 28.87 |
| ATOM | 2336 | CG1 | VAL | A | 293 | −3.244 | 80.086 | 21.346 | 1.00 | 29.10 |
| ATOM | 2337 | CG2 | VAL | A | 293 | −3.157 | 82.350 | 20.340 | 1.00 | 28.79 |
| ATOM | 2338 | C | VAL | A | 293 | −4.754 | 79.193 | 18.925 | 1.00 | 33.08 |
| ATOM | 2339 | O | VAL | A | 293 | −5.971 | 79.366 | 18.990 | 1.00 | 33.77 |
| ATOM | 2340 | N | ILE | A | 294 | −4.187 | 78.002 | 18.790 | 1.00 | 34.36 |
| ATOM | 2341 | CA | ILE | A | 294 | −4.952 | 76.770 | 18.784 | 1.00 | 33.14 |
| ATOM | 2342 | CB | ILE | A | 294 | −4.754 | 76.000 | 17.458 | 1.00 | 33.14 |
| ATOM | 2343 | CG2 | ILE | A | 294 | −5.426 | 74.629 | 17.518 | 1.00 | 28.09 |
| ATOM | 2344 | CG1 | ILE | A | 294 | −5.318 | 76.832 | 16.303 | 1.00 | 29.00 |
| ATOM | 2345 | CD1 | ILE | A | 294 | −5.328 | 76.129 | 14.973 | 1.00 | 30.92 |
| ATOM | 2346 | C | ILE | A | 294 | −4.476 | 75.952 | 19.983 | 1.00 | 36.82 |
| ATOM | 2347 | O | ILE | A | 294 | −3.277 | 75.882 | 20.273 | 1.00 | 40.13 |
| ATOM | 2348 | N | ASP | A | 295 | −5.427 | 75.385 | 20.710 | 1.00 | 37.77 |
| ATOM | 2349 | CA | ASP | A | 295 | −5.124 | 74.604 | 21.889 | 1.00 | 36.56 |
| ATOM | 2350 | CB | ASP | A | 295 | −6.346 | 74.499 | 22.783 | 1.00 | 42.24 |
| ATOM | 2351 | CG | ASP | A | 295 | −6.163 | 75.234 | 24.071 | 1.00 | 50.87 |
| ATOM | 2352 | OD1 | ASP | A | 295 | −6.049 | 74.565 | 25.117 | 1.00 | 60.02 |
| ATOM | 2353 | OD2 | ASP | A | 295 | −6.092 | 76.481 | 24.038 | 1.00 | 55.86 |
| ATOM | 2354 | C | ASP | A | 295 | −4.655 | 73.223 | 21.544 | 1.00 | 35.69 |
| ATOM | 2355 | O | ASP | A | 295 | −5.384 | 72.453 | 20.928 | 1.00 | 35.08 |
| ATOM | 2356 | N | PRO | A | 296 | −3.431 | 72.879 | 21.955 | 1.00 | 35.13 |
| ATOM | 2357 | CD | PRO | A | 296 | −2.488 | 73.736 | 22.692 | 1.00 | 34.11 |
| ATOM | 2358 | CA | PRO | A | 296 | −2.847 | 71.563 | 21.690 | 1.00 | 35.32 |
| ATOM | 2359 | CB | PRO | A | 296 | −1.478 | 71.653 | 22.368 | 1.00 | 33.77 |
| ATOM | 2360 | CG | PRO | A | 296 | −1.169 | 73.116 | 22.339 | 1.00 | 35.72 |
| ATOM | 2361 | C | PRO | A | 296 | −3.689 | 70.463 | 22.327 | 1.00 | 38.35 |
| ATOM | 2362 | O | PRO | A | 296 | −4.269 | 70.653 | 23.394 | 1.00 | 41.07 |
| ATOM | 2363 | N | PHE | A | 297 | −3.706 | 69.300 | 21.697 | 1.00 | 38.55 |
| ATOM | 2364 | CA | PHE | A | 297 | −4.455 | 68.180 | 22.208 | 1.00 | 37.27 |
| ATOM | 2365 | CB | PHE | A | 297 | −5.877 | 68.209 | 21.654 | 1.00 | 34.45 |
| ATOM | 2366 | CG | PHE | A | 297 | −5.957 | 68.187 | 20.151 | 1.00 | 31.82 |
| ATOM | 2367 | CD1 | PHE | A | 297 | −6.324 | 67.025 | 19.475 | 1.00 | 30.97 |
| ATOM | 2368 | CD2 | PHE | A | 297 | −5.712 | 69.339 | 19.414 | 1.00 | 31.48 |
| ATOM | 2369 | CE1 | PHE | A | 297 | −6.445 | 67.018 | 18.083 | 1.00 | 30.11 |
| ATOM | 2370 | CE2 | PHE | A | 297 | −5.832 | 69.338 | 18.023 | 1.00 | 29.97 |
| ATOM | 2371 | CZ | PHE | A | 297 | −6.200 | 68.177 | 17.358 | 1.00 | 30.13 |
| ATOM | 2372 | C | PHE | A | 297 | −3.770 | 66.887 | 21.809 | 1.00 | 40.88 |
| ATOM | 2373 | O | PHE | A | 297 | −2.954 | 66.865 | 20.891 | 1.00 | 43.86 |
| ATOM | 2374 | N | ASP | A | 298 | −4.064 | 65.806 | 22.511 | 1.00 | 44.53 |
| ATOM | 2375 | CA | ASP | A | 298 | −3.466 | 64.534 | 22.167 | 1.00 | 48.48 |
| ATOM | 2376 | CB | ASP | A | 298 | −2.590 | 64.015 | 23.295 | 1.00 | 52.93 |
| ATOM | 2377 | CG | ASP | A | 298 | −1.808 | 62.778 | 22.898 | 1.00 | 57.44 |
| ATOM | 2378 | OD1 | ASP | A | 298 | −2.020 | 62.254 | 21.778 | 1.00 | 54.35 |
| ATOM | 2379 | OD2 | ASP | A | 298 | −0.964 | 62.333 | 23.705 | 1.00 | 66.50 |
| ATOM | 2380 | C | ASP | A | 298 | −4.584 | 63.552 | 21.907 | 1.00 | 50.22 |
| ATOM | 2381 | O | ASP | A | 298 | −5.215 | 63.067 | 22.827 | 1.00 | 49.77 |
| ATOM | 2382 | N | PRO | A | 299 | −4.789 | 63.199 | 20.630 | 1.00 | 52.46 |
| ATOM | 2383 | CD | PRO | A | 299 | −4.105 | 63.730 | 19.439 | 1.00 | 52.72 |
| ATOM | 2384 | CA | PRO | A | 299 | −5.835 | 62.266 | 20.230 | 1.00 | 56.64 |
| ATOM | 2385 | CB | PRO | A | 299 | −5.663 | 62.206 | 18.717 | 1.00 | 54.35 |
| ATOM | 2386 | CG | PRO | A | 299 | −5.165 | 63.564 | 18.388 | 1.00 | 50.07 |
| ATOM | 2387 | C | PRO | A | 299 | −5.648 | 60.898 | 20.868 | 1.00 | 63.86 |
| ATOM | 2388 | O | PRO | A | 299 | −6.492 | 60.011 | 20.687 | 1.00 | 69.53 |
| ATOM | 2389 | N | MG | A | 300 | −4.535 | 60.712 | 21.580 | 1.00 | 68.69 |
| ATOM | 2390 | CA | ARG | A | 300 | −4.231 | 59.449 | 22.250 | 1.00 | 73.13 |
| ATOM | 2391 | CB | ARG | A | 300 | −2.731 | 59.153 | 22.191 | 1.00 | 73.05 |
| ATOM | 2392 | CG | ARG | A | 300 | −2.202 | 58.825 | 20.810 | 1.00 | 75.18 |
| ATOM | 2393 | CD | ARG | A | 300 | −0.682 | 58.842 | 20.790 | 1.00 | 72.55 |
| ATOM | 2394 | NE | ARG | A | 300 | −0.165 | 60.144 | 21.181 | 1.00 | 71.35 |
| ATOM | 2395 | CZ | ARG | A | 300 | 0.867 | 60.748 | 20.595 | 1.00 | 71.94 |
| ATOM | 2396 | NH1 | ARG | A | 300 | 1.506 | 60.171 | 19.579 | 1.00 | 73.46 |
| ATOM | 2397 | NH2 | ARG | A | 300 | 1.274 | 61.934 | 21.032 | 1.00 | 71.10 |
| ATOM | 2398 | C | ARG | A | 300 | −4.685 | 59.414 | 23.708 | 1.00 | 76.43 |
| ATOM | 2399 | O | ARG | A | 300 | −4.552 | 58.390 | 24.374 | 1.00 | 80.12 |
| ATOM | 2400 | N | GLU | A | 301 | −5.202 | 60.536 | 24.196 | 1.00 | 77.10 |
| ATOM | 2401 | CA | GLU | A | 301 | −5.687 | 60.596 | 25.562 | 1.00 | 81.37 |
| ATOM | 2402 | CB | GLU | A | 301 | −4.984 | 61.711 | 26.331 | 1.00 | 82.38 |
| ATOM | 2403 | CG | GLU | A | 301 | −3.474 | 61.523 | 26.422 | 1.00 | 89.06 |
| ATOM | 2404 | CD | GLU | A | 301 | −2.918 | 62.004 | 27.750 | 1.00 | 94.30 |
| ATOM | 2405 | OE1 | GLU | A | 301 | −3.152 | 63.179 | 28.115 | 1.00 | 97.83 |
| ATOM | 2406 | OE2 | GLU | A | 301 | −2.241 | 61.201 | 28.425 | 1.00 | 97.36 |
| ATOM | 2407 | C | GLU | A | 301 | −7.193 | 60.781 | 25.642 | 1.00 | 84.64 |
| ATOM | 2408 | O | GLU | A | 301 | −7.779 | 61.498 | 24.836 | 1.00 | 83.56 |
| ATOM | 2409 | N | PRO | A | 302 | −7.849 | 60.122 | 26.611 | 1.00 | 88.29 |
| ATOM | 2410 | CD | PRO | A | 302 | −7.263 | 59.114 | 27.527 | 1.00 | 89.03 |
| ATOM | 2411 | CA | PRO | A | 302 | −9.303 | 60.201 | 26.800 | 1.00 | 87.98 |
| ATOM | 2412 | CB | PRO | A | 302 | −9.521 | 59.416 | 28.095 | 1.00 | 88.81 |
| ATOM | 2413 | CG | PRO | A | 302 | −8.478 | 58.337 | 27.978 | 1.00 | 88.81 |
| ATOM | 2414 | C | PRO | A | 302 | −9.804 | 61.652 | 26.925 | 1.00 | 86.15 |
| ATOM | 2415 | O | PRO | A | 302 | −10.737 | 62.060 | 26.236 | 1.00 | 85.57 |
| ATOM | 2416 | N | ASN | A | 303 | −9.184 | 62.425 | 27.817 | 1.00 | 84.86 |
| ATOM | 2417 | CA | ASN | A | 303 | −9.563 | 63.822 | 27.985 | 1.00 | 85.24 |
| ATOM | 2418 | CB | ASN | A | 303 | −8.929 | 64.404 | 29.254 | 1.00 | 89.02 |
| ATOM | 2419 | CG | ASN | A | 303 | −9.217 | 65.900 | 29.433 | 1.00 | 93.08 |
| ATOM | 2420 | OD1 | ASN | A | 303 | −8.501 | 66.589 | 30.150 | 1.00 | 95.61 |
| ATOM | 2421 | ND2 | ASN | A | 303 | −10.242 | 66.401 | 28.755 | 1.00 | 95.94 |
| ATOM | 2422 | C | ASN | A | 303 | −9.073 | 64.602 | 26.773 | 1.00 | 83.46 |
| ATOM | 2423 | O | ASN | A | 303 | −9.678 | 65.601 | 26.377 | 1.00 | 82.51 |
| ATOM | 2424 | N | GLY | A | 304 | −8.001 | 64.099 | 26.169 | 1.00 | 82.29 |
| ATOM | 2425 | CA | GLY | A | 304 | −7.413 | 64.745 | 25.016 | 1.00 | 79.23 |
| ATOM | 2426 | C | GLY | A | 304 | −6.639 | 65.945 | 25.513 | 1.00 | 77.89 |
| ATOM | 2427 | O | GLY | A | 304 | −6.503 | 66.945 | 24.802 | 1.00 | 78.97 |
| ATOM | 2428 | N | LYS | A | 305 | −6.156 | 65.855 | 26.748 | 1.00 | 76.87 |
| ATOM | 2429 | CA | LYS | A | 305 | −5.403 | 66.938 | 27.348 | 1.00 | 76.76 |
| ATOM | 2430 | CB | LYS | A | 305 | −5.585 | 66.962 | 28.880 | 1.00 | 78.46 |
| ATOM | 2431 | CG | LYS | A | 305 | −4.408 | 66.440 | 29.691 | 1.00 | 82.23 |
| ATOM | 2432 | CD | LYS | A | 305 | −3.751 | 67.539 | 30.519 | 1.00 | 85.07 |
| ATOM | 2433 | CE | LYS | A | 305 | −2.573 | 66.997 | 31.311 | 1.00 | 88.93 |
| ATOM | 2434 | NZ | LYS | A | 305 | −2.157 | 67.917 | 32.403 | 1.00 | 93.14 |
| ATOM | 2435 | C | LYS | A | 305 | −3.949 | 66.755 | 26.972 | 1.00 | 73.92 |
| ATOM | 2436 | O | LYS | A | 305 | −3.474 | 65.623 | 26.867 | 1.00 | 73.27 |
| ATOM | 2437 | N | SER | A | 306 | −3.237 | 67.855 | 26.797 | 1.00 | 72.39 |
| ATOM | 2438 | CA | SER | A | 306 | −1.844 | 67.787 | 26.417 | 1.00 | 73.15 |
| ATOM | 2439 | CB | SER | A | 306 | −1.656 | 68.461 | 25.061 | 1.00 | 72.57 |
| ATOM | 2440 | OG | SER | A | 306 | −2.237 | 69.762 | 25.069 | 1.00 | 70.16 |
| ATOM | 2441 | C | SER | A | 306 | −0.982 | 68.474 | 27.445 | 1.00 | 73.60 |
| ATOM | 2442 | O | SER | A | 306 | −1.481 | 69.180 | 28.328 | 1.00 | 71.26 |
| ATOM | 2443 | N | ASP | A | 307 | 0.314 | 68.208 | 27.363 | 1.00 | 76.59 |
| ATOM | 2444 | CA | ASP | A | 307 | 1.281 | 68.827 | 28.260 | 1.00 | 78.33 |
| ATOM | 2445 | CB | ASP | A | 307 | 2.480 | 67.891 | 28.515 | 1.00 | 84.92 |
| ATOM | 2446 | CG | ASP | A | 307 | 3.022 | 67.239 | 27.236 | 1.00 | 89.57 |
| ATOM | 2447 | OD1 | ASP | A | 307 | 3.809 | 67.902 | 26.515 | 1.00 | 90.72 |
| ATOM | 2448 | OD2 | ASP | A | 307 | 2.672 | 66.065 | 26.956 | 1.00 | 90.72 |
| ATOM | 2449 | C | ASP | A | 307 | 1.734 | 70.130 | 27.593 | 1.00 | 74.93 |
| ATOM | 2450 | O | ASP | A | 307 | 1.883 | 71.163 | 28.254 | 1.00 | 73.18 |
| ATOM | 2451 | N | ARG | A | 308 | 1.893 | 70.072 | 26.268 | 1.00 | 70.05 |
| ATOM | 2452 | CA | ARG | A | 308 | 2.327 | 71.212 | 25.471 | 1.00 | 64.62 |
| ATOM | 2453 | CB | ARG | A | 308 | 2.295 | 70.877 | 23.986 | 1.00 | 64.91 |
| ATOM | 2454 | CG | ARG | A | 308 | 3.177 | 69.738 | 23.570 | 1.00 | 65.23 |
| ATOM | 2455 | CD | ARG | A | 308 | 3.284 | 69.720 | 22.067 | 1.00 | 65.31 |
| ATOM | 2456 | NE | ARG | A | 308 | 3.889 | 68.494 | 21.573 | 1.00 | 68.52 |
| ATOM | 2457 | CZ | ARG | A | 308 | 5.145 | 68.139 | 21.800 | 1.00 | 69.44 |
| ATOM | 2458 | NH1 | ARG | A | 308 | 5.939 | 68.925 | 22.515 | 1.00 | 70.73 |
| ATOM | 2459 | NH2 | ARG | A | 308 | 5.596 | 66.983 | 21.334 | 1.00 | 71.60 |
| ATOM | 2460 | C | ARG | A | 308 | 1.422 | 72.394 | 25.695 | 1.00 | 60.56 |
| ATOM | 2461 | O | ARG | A | 308 | 0.211 | 72.243 | 25.786 | 1.00 | 62.91 |
| ATOM | 2462 | N | GLU | A | 309 | 2.016 | 73.576 | 25.721 | 1.00 | 58.75 |
| ATOM | 2463 | CA | GLU | A | 309 | 1.278 | 74.807 | 25.937 | 1.00 | 59.53 |
| ATOM | 2464 | CB | GLU | A | 309 | 2.155 | 75.795 | 26.707 | 1.00 | 64.51 |
| ATOM | 2465 | CG | GLU | A | 309 | 2.863 | 75.195 | 27.909 | 1.00 | 72.73 |
| ATOM | 2466 | CD | GLU | A | 309 | 4.037 | 76.041 | 28.375 | 1.00 | 78.39 |
| ATOM | 2467 | OE1 | GLU | A | 309 | 5.196 | 75.650 | 28.107 | 1.00 | 80.45 |
| ATOM | 2468 | OE2 | GLU | A | 309 | 3.802 | 77.091 | 29.013 | 1.00 | 81.42 |
| ATOM | 2469 | C | GLU | A | 309 | 0.895 | 75.423 | 24.595 | 1.00 | 58.18 |
| ATOM | 2470 | O | GLU | A | 309 | 1.550 | 75.172 | 23.576 | 1.00 | 56.69 |
| ATOM | 2471 | N | PRO | A | 310 | −0.202 | 76.204 | 24.566 | 1.00 | 57.07 |
| ATOM | 2472 | CD | PRO | A | 310 | −1.130 | 76.528 | 25.662 | 1.00 | 58.98 |
| ATOM | 2473 | CA | PRO | A | 310 | −0.639 | 76.848 | 23.325 | 1.00 | 54.68 |
| ATOM | 2474 | CB | PRO | A | 310 | −1.898 | 77.610 | 23.755 | 1.00 | 56.45 |
| ATOM | 2475 | CG | PRO | A | 310 | −1.671 | 77.867 | 25.214 | 1.00 | 57.32 |
| ATOM | 2476 | C | PRO | A | 310 | 0.456 | 77.787 | 22.831 | 1.00 | 49.05 |
| ATOM | 2477 | O | PRO | A | 310 | 1.136 | 78.437 | 23.631 | 1.00 | 48.09 |
| ATOM | 2478 | N | LEU | A | 311 | 0.617 | 77.835 | 21.512 | 1.00 | 43.40 |
| ATOM | 2479 | CA | LEU | A | 311 | 1.626 | 78.650 | 20.859 | 1.00 | 36.12 |
| ATOM | 2480 | CB | LEU | A | 311 | 2.661 | 77.726 | 20.204 | 1.00 | 32.22 |
| ATOM | 2481 | CG | LEU | A | 311 | 3.919 | 78.324 | 19.578 | 1.00 | 31.79 |
| ATOM | 2482 | CD1 | LEU | A | 311 | 4.582 | 79.300 | 20.539 | 1.00 | 28.33 |
| ATOM | 2483 | CD2 | LEU | A | 311 | 4.862 | 77.194 | 19.229 | 1.00 | 26.30 |
| ATOM | 2484 | C | LEU | A | 311 | 0.936 | 79.478 | 19.793 | 1.00 | 32.51 |
| ATOM | 2485 | O | LEU | A | 311 | 0.300 | 78.915 | 18.903 | 1.00 | 35.00 |
| ATOM | 2486 | N | SER | A | 312 | 1.012 | 80.804 | 19.899 | 1.00 | 27.12 |
| ATOM | 2487 | CA | SER | A | 312 | 0.386 | 81.654 | 18.894 | 1.00 | 26.22 |
| ATOM | 2488 | CB | SER | A | 312 | 0.324 | 83.123 | 19.339 | 1.00 | 22.14 |
| ATOM | 2489 | OG | SER | A | 312 | 1.479 | 83.859 | 19.000 | 1.00 | 31.44 |
| ATOM | 2490 | C | SER | A | 312 | 1.205 | 81.464 | 17.621 | 1.00 | 30.09 |
| ATOM | 2491 | O | SER | A | 312 | 2.438 | 81.349 | 17.671 | 1.00 | 34.62 |
| ATOM | 2492 | N | TYR | A | 313 | 0.526 | 81.399 | 16.483 | 1.00 | 30.05 |
| ATOM | 2493 | CA | TYR | A | 313 | 1.208 | 81.164 | 15.222 | 1.00 | 26.78 |
| ATOM | 2494 | CB | TYR | A | 313 | 0.221 | 81.101 | 14.070 | 1.00 | 21.90 |
| ATOM | 2495 | CG | TYR | A | 313 | 0.740 | 80.223 | 12.968 | 1.00 | 23.94 |
| ATOM | 2496 | CD1 | TYR | A | 313 | 0.464 | 78.868 | 12.972 | 1.00 | 20.72 |
| ATOM | 2497 | CE1 | TYR | A | 313 | 0.991 | 78.032 | 12.022 | 1.00 | 22.06 |
| ATOM | 2498 | CD2 | TYR | A | 313 | 1.555 | 80.729 | 11.951 | 1.00 | 16.61 |
| ATOM | 2499 | CE2 | TYR | A | 313 | 2.091 | 79.891 | 10.996 | 1.00 | 16.69 |
| ATOM | 2500 | CZ | TYR | A | 313 | 1.792 | 78.541 | 11.041 | 1.00 | 17.14 |
| ATOM | 2501 | OH | TYR | A | 313 | 2.294 | 77.666 | 10.123 | 1.00 | 24.17 |
| ATOM | 2502 | C | TYR | A | 313 | 2.299 | 82.153 | 14.895 | 1.00 | 25.77 |
| ATOM | 2503 | O | TYR | A | 313 | 3.326 | 81.778 | 14.334 | 1.00 | 23.63 |
| ATOM | 2504 | N | GLY | A | 314 | 2.071 | 83.415 | 15.238 | 1.00 | 28.58 |
| ATOM | 2505 | CA | GLY | A | 314 | 3.053 | 84.444 | 14.965 | 1.00 | 34.94 |
| ATOM | 2506 | C | GLY | A | 314 | 4.370 | 84.186 | 15.674 | 1.00 | 37.23 |
| ATOM | 2507 | O | GLY | A | 314 | 5.434 | 84.453 | 15.117 | 1.00 | 41.94 |
| ATOM | 2508 | N | ASP | A | 315 | 4.301 | 83.683 | 16.906 | 1.00 | 37.16 |
| ATOM | 2509 | CA | ASP | A | 315 | 5.498 | 83.388 | 17.682 | 1.00 | 33.44 |
| ATOM | 2510 | CB | ASP | A | 315 | 5.162 | 83.140 | 19.157 | 1.00 | 37.41 |
| ATOM | 2511 | CG | ASP | A | 315 | 4.707 | 84.406 | 19.881 | 1.00 | 40.95 |
| ATOM | 2512 | OD1 | ASP | A | 315 | 3.906 | 84.295 | 20.835 | 1.00 | 48.93 |
| ATOM | 2513 | OD2 | ASP | A | 315 | 5.147 | 85.515 | 19.504 | 1.00 | 45.51 |
| ATOM | 2514 | C | ASP | A | 315 | 6.147 | 82.172 | 17.074 | 1.00 | 28.81 |
| ATOM | 2515 | O | ASP | A | 315 | 7.357 | 82.139 | 16.893 | 1.00 | 32.18 |
| ATOM | 2516 | N | TYR | A | 316 | 5.333 | 81.179 | 16.746 | 1.00 | 26.31 |
| ATOM | 2517 | CA | TYR | A | 316 | 5.823 | 79.963 | 16.116 | 1.00 | 25.28 |
| ATOM | 2518 | CB | TYR | A | 316 | 4.646 | 79.064 | 15.709 | 1.00 | 23.80 |
| ATOM | 2519 | CG | TYR | A | 316 | 4.986 | 77.997 | 14.682 | 1.00 | 28.01 |
| ATOM | 2520 | CD1 | TYR | A | 316 | 5.604 | 76.802 | 15.061 | 1.00 | 26.83 |
| ATOM | 2521 | CE1 | TYR | A | 316 | 5.903 | 75.810 | 14.106 | 1.00 | 25.18 |
| ATOM | 2522 | CD2 | TYR | A | 316 | 4.682 | 78.177 | 13.323 | 1.00 | 22.23 |
| ATOM | 2523 | CE2 | TYR | A | 316 | 4.981 | 77.194 | 12.372 | 1.00 | 16.07 |
| ATOM | 2524 | CZ | TYR | A | 316 | 5.586 | 76.020 | 12.769 | 1.00 | 19.33 |
| ATOM | 2525 | 08 | TYR | A | 316 | 5.850 | 75.040 | 11.843 | 1.00 | 22.12 |
| ATOM | 2526 | C | TYR | A | 316 | 6.625 | 80.333 | 14.872 | 1.00 | 26.20 |
| ATOM | 2527 | O | TYR | A | 316 | 7.812 | 80.010 | 14.766 | 1.00 | 23.86 |
| ATOM | 2528 | N | LEU | A | 317 | 5.977 | 81.062 | 13.966 | 1.00 | 26.87 |
| ATOM | 2529 | CA | LEU | A | 317 | 6.579 | 81.454 | 12.705 | 1.00 | 26.33 |
| ATOM | 2530 | CB | LEU | A | 317 | 5.548 | 82.112 | 11.783 | 1.00 | 22.99 |
| ATOM | 2531 | CG | LEU | A | 317 | 6.032 | 82.167 | 10.334 | 1.00 | 17.66 |
| ATOM | 2532 | CD1 | LEU | A | 317 | 5.962 | 80.780 | 9.722 | 1.00 | 16.43 |
| ATOM | 2533 | CD2 | LEU | A | 317 | 5.205 | 83.147 | 9.549 | 1.00 | 13.47 |
| ATOM | 2534 | C | LEU | A | 317 | 7.801 | 82.340 | 12.830 | 1.00 | 25.42 |
| ATOM | 2535 | O | LEU | A | 317 | 8.781 | 82.125 | 12.134 | 1.00 | 29.40 |
| ATOM | 2536 | N | GLN | A | 318 | 7.753 | 83.341 | 13.696 | 1.00 | 26.38 |
| ATOM | 2537 | CA | GLN | A | 318 | 8.891 | 84.226 | 13.846 | 1.00 | 29.10 |
| ATOM | 2538 | CB | GLN | A | 318 | 8.643 | 85.254 | 14.933 | 1.00 | 34.57 |
| ATOM | 2539 | CG | GLN | A | 318 | 7.722 | 86.361 | 14.557 | 1.00 | 45.89 |
| ATOM | 2540 | CD | GLN | A | 318 | 7.422 | 87.230 | 15.744 | 1.00 | 54.11 |
| ATOM | 2541 | OE1 | GLN | A | 318 | 8.276 | 87.996 | 16.198 | 1.00 | 60.92 |
| ATOM | 2542 | NE2 | GLN | A | 318 | 6.224 | 87.084 | 16.292 | 1.00 | 56.69 |
| ATOM | 2543 | C | GLN | A | 318 | 10.114 | 83.429 | 14.215 | 1.00 | 32.19 |
| ATOM | 2544 | O | GLN | A | 318 | 11.147 | 83.529 | 13.560 | 1.00 | 34.97 |
| ATOM | 2545 | N | ASN | A | 319 | 9.967 | 82.589 | 15.231 | 1.00 | 33.42 |
| ATOM | 2546 | CA | ASN | A | 319 | 11.076 | 81.780 | 15.711 | 1.00 | 38.92 |
| ATOM | 2547 | CB | ASN | A | 319 | 10.751 | 81.192 | 17.088 | 1.00 | 45.44 |
| ATOM | 2548 | CG | ASN | A | 319 | 10.635 | 82.276 | 18.174 | 1.00 | 54.83 |
| ATOM | 2549 | OD1 | ASN | A | 319 | 11.612 | 82.952 | 18.502 | 1.00 | 56.44 |
| ATOM | 2550 | ND2 | ASN | A | 319 | 9.429 | 82.470 | 18.702 | 1.00 | 59.48 |
| ATOM | 2551 | C | ASN | A | 319 | 11.506 | 80.705 | 14.725 | 1.00 | 40.03 |
| ATOM | 2552 | O | ASN | A | 319 | 12.703 | 80.502 | 14.494 | 1.00 | 44.50 |
| ATOM | 2553 | N | GLY | A | 320 | 10.531 | 80.058 | 14.100 | 1.00 | 39.74 |
| ATOM | 2554 | CA | GLY | A | 320 | 10.827 | 79.022 | 13.130 | 1.00 | 35.73 |
| ATOM | 2555 | C | GLY | A | 320 | 11.611 | 79.582 | 11.962 | 1.00 | 35.37 |
| ATOM | 2556 | O | GLY | A | 320 | 12.536 | 78.951 | 11.471 | 1.00 | 37.50 |
| ATOM | 2557 | N | LEU | A | 321 | 11.270 | 80.786 | 11.530 | 1.00 | 34.76 |
| ATOM | 2558 | CA | LEU | A | 321 | 11.967 | 81.393 | 10.415 | 1.00 | 39.63 |
| ATOM | 2559 | CB | LEU | A | 321 | 11.191 | 82.583 | 9.877 | 1.00 | 36.97 |
| ATOM | 2560 | CG | LEU | A | 321 | 9.901 | 82.168 | 9.187 | 1.00 | 38.18 |
| ATOM | 2561 | CD1 | LEU | A | 321 | 9.271 | 83.392 | 8.570 | 1.00 | 38.33 |
| ATOM | 2562 | CD2 | LEU | A | 321 | 10.181 | 81.108 | 8.134 | 1.00 | 35.19 |
| ATOM | 2563 | C | LEU | A | 321 | 13.382 | 81.805 | 10.757 | 1.00 | 44.08 |
| ATOM | 2564 | O | LEU | A | 321 | 14.287 | 81.606 | 9.953 | 1.00 | 46.65 |
| ATOM | 2565 | N | VAL | A | 322 | 13.581 | 82.378 | 11.941 | 1.00 | 49.49 |
| ATOM | 2566 | CA | VAL | A | 322 | 14.923 | 82.806 | 12.345 | 1.00 | 53.83 |
| ATOM | 2567 | CB | VAL | A | 322 | 14.939 | 83.617 | 13.671 | 1.00 | 53.89 |
| ATOM | 2568 | CG1 | VAL | A | 322 | 13.945 | 84.767 | 13.615 | 1.00 | 53.18 |
| ATOM | 2569 | CG2 | VAL | A | 322 | 14.698 | 82.698 | 14.869 | 1.00 | 55.17 |
| ATOM | 2570 | C | VAL | A | 322 | 15.894 | 81.645 | 12.511 | 1.00 | 54.31 |
| ATOM | 2571 | O | VAL | A | 322 | 17.104 | 81.848 | 12.478 | 1.00 | 59.46 |
| ATOM | 2572 | N | SER | A | 323 | 15.378 | 80.452 | 12.773 | 1.00 | 52.61 |
| ATOM | 2573 | CA | SER | A | 323 | 16.254 | 79.309 | 12.940 | 1.00 | 53.68 |
| ATOM | 2574 | CB | SER | A | 323 | 15.480 | 78.096 | 13.468 | 1.00 | 55.08 |
| ATOM | 2575 | OG | SER | A | 323 | 14.499 | 77.644 | 12.551 | 1.00 | 52.98 |
| ATOM | 2576 | C | SER | A | 323 | 16.913 | 78.965 | 11.616 | 1.00 | 54.49 |
| ATOM | 2577 | O | SER | A | 323 | 18.061 | 78.523 | 11.584 | 1.00 | 58.60 |
| ATOM | 2578 | N | LEU | A | 324 | 16.194 | 79.186 | 10.522 | 1.00 | 55.84 |
| ATOM | 2579 | CA | LEU | A | 324 | 16.725 | 78.861 | 9.207 | 1.00 | 56.10 |
| ATOM | 2580 | CB | LEU | A | 324 | 15.596 | 78.485 | 8.229 | 1.00 | 52.73 |
| ATOM | 2581 | CG | LEU | A | 324 | 14.383 | 79.396 | 8.017 | 1.00 | 51.08 |
| ATOM | 2582 | CD1 | LEU | A | 324 | 14.679 | 80.431 | 6.945 | 1.00 | 54.72 |
| ATOM | 2583 | CD2 | LEU | A | 324 | 13.183 | 78.561 | 7.606 | 1.00 | 50.43 |
| ATOM | 2584 | C | LEU | A | 324 | 17.637 | 79.928 | 8.625 | 1.00 | 57.85 |
| ATOM | 2585 | O | LEU | A | 324 | 17.696 | 81.050 | 9.116 | 1.00 | 55.56 |
| ATOM | 2586 | N | ILE | A | 325 | 18.379 | 79.536 | 7.595 | 1.00 | 62.83 |
| ATOM | 2587 | CA | ILE | A | 325 | 19.307 | 80.428 | 6.910 | 1.00 | 64.52 |
| ATOM | 2588 | CB | ILE | A | 325 | 20.264 | 79.668 | 5.969 | 1.00 | 64.56 |
| ATOM | 2589 | CG2 | ILE | A | 325 | 21.516 | 80.496 | 5.747 | 1.00 | 65.36 |
| ATOM | 2590 | CG1 | ILE | A | 325 | 20.591 | 78.271 | 6.516 | 1.00 | 65.53 |
| ATOM | 2591 | CD1 | ILE | A | 325 | 19.563 | 77.197 | 6.136 | 1.00 | 67.86 |
| ATOM | 2592 | C | ILE | A | 325 | 18.501 | 81.383 | 6.034 | 1.00 | 65.56 |
| ATOM | 2593 | O | ILE | A | 325 | 17.808 | 80.957 | 5.110 | 1.00 | 66.01 |
| ATOM | 2594 | N | ASN | A | 326 | 18.621 | 82.673 | 6.312 | 1.00 | 67.38 |
| ATOM | 2595 | CA | ASN | A | 326 | 17.899 | 83.696 | 5.559 | 1.00 | 68.33 |
| ATOM | 2596 | CB | ASN | A | 326 | 18.205 | 85.097 | 6.105 | 1.00 | 73.56 |
| ATOM | 2597 | CG | ASN | A | 326 | 17.632 | 85.325 | 7.495 | 1.00 | 78.94 |
| ATOM | 2598 | OD1 | ASN | A | 326 | 17.224 | 84.386 | 8.171 | 1.00 | 83.34 |
| ATOM | 2599 | ND2 | ASN | A | 326 | 17.590 | 86.582 | 7.920 | 1.00 | 79.40 |
| ATOM | 2600 | C | ASN | A | 326 | 18.169 | 83.663 | 4.068 | 1.00 | 65.53 |
| ATOM | 2601 | O | ASN | A | 326 | 17.254 | 83.861 | 3.274 | 1.00 | 67.11 |
| ATOM | 2602 | N | LYS | A | 327 | 19.413 | 83.404 | 3.681 | 1.00 | 59.01 |
| ATOM | 2603 | CA | LYS | A | 327 | 19.761 | 83.355 | 2.263 | 1.00 | 54.20 |
| ATOM | 2604 | CB | LYS | A | 327 | 21.277 | 83.218 | 2.095 | 1.00 | 53.73 |
| ATOM | 2605 | CG | LYS | A | 327 | 22.021 | 84.515 | 2.327 | 1.00 | 53.84 |
| ATOM | 2606 | CD | LYS | A | 327 | 23.511 | 84.317 | 2.266 | 1.00 | 60.90 |
| ATOM | 2607 | CE | LYS | A | 327 | 24.262 | 85.605 | 2.584 | 1.00 | 61.87 |
| ATOM | 2608 | NZ | LYS | A | 327 | 24.203 | 86.582 | 1.467 | 1.00 | 58.17 |
| ATOM | 2609 | C | LYS | A | 327 | 19.033 | 82.256 | 1.500 | 1.00 | 51.14 |
| ATOM | 2610 | O | LYS | A | 327 | 18.610 | 82.472 | 0.370 | 1.00 | 50.58 |
| ATOM | 2611 | N | ASN | A | 328 | 18.868 | 81.088 | 2.109 | 1.00 | 51.00 |
| ATOM | 2612 | CA | ASN | A | 328 | 18.186 | 79.980 | 1.439 | 1.00 | 56.30 |
| ATOM | 2613 | CB | ASN | A | 328 | 18.717 | 78.637 | 1.947 | 1.00 | 59.34 |
| ATOM | 2614 | CG | ASN | A | 328 | 20.104 | 78.345 | 1.410 | 1.00 | 62.04 |
| ATOM | 2615 | OD1 | ASN | A | 328 | 21.052 | 79.056 | 1.725 | 1.00 | 64.52 |
| ATOM | 2616 | ND2 | ASN | A | 328 | 20.226 | 77.327 | 0.562 | 1.00 | 65.59 |
| ATOM | 2617 | C | ASN | A | 328 | 16.657 | 80.013 | 1.498 | 1.00 | 56.83 |
| ATOM | 2618 | O | ASN | A | 328 | 15.976 | 79.760 | 0.505 | 1.00 | 57.74 |
| ATOM | 2619 | N | GLY | A | 329 | 16.117 | 80.291 | 2.684 | 1.00 | 59.02 |
| ATOM | 2620 | CA | GLY | A | 329 | 14.678 | 80.340 | 2.864 | 1.00 | 57.93 |
| ATOM | 2621 | C | GLY | A | 329 | 13.973 | 79.011 | 2.645 | 1.00 | 61.61 |
| ATOM | 2622 | O | GLY | A | 329 | 14.604 | 77.939 | 2.645 | 1.00 | 63.66 |
| ATOM | 2623 | N | GLN | A | 330 | 12.665 | 79.134 | 2.426 | 1.00 | 63.44 |
| ATOM | 2624 | CA | GLN | A | 330 | 11.647 | 78.088 | 2.186 | 1.00 | 62.51 |
| ATOM | 2625 | CB | GLN | A | 330 | 12.152 | 76.612 | 2.170 | 1.00 | 61.84 |
| ATOM | 2626 | CG | GLN | A | 330 | 11.146 | 75.609 | 1.460 | 1.00 | 62.53 |
| ATOM | 2627 | CD | GLN | A | 330 | 11.591 | 74.116 | 1.364 | 1.00 | 61.91 |
| ATOM | 2628 | OE1 | GLN | A | 330 | 10.948 | 73.200 | 1.946 | 1.00 | 56.42 |
| ATOM | 2629 | NE2 | GLN | A | 330 | 12.658 | 73.864 | 0.605 | 1.00 | 56.02 |
| ATOM | 2630 | C | GLN | A | 330 | 10.703 | 78.366 | 3.357 | 1.00 | 61.76 |
| ATOM | 2631 | O | GLN | A | 330 | 11.044 | 78.121 | 4.511 | 1.00 | 61.59 |
| ATOM | 2632 | N | THR | A | 331 | 9.623 | 79.069 | 3.018 | 1.00 | 61.83 |
| ATOM | 2633 | CA | THR | A | 331 | 8.542 | 79.549 | 3.891 | 1.00 | 57.39 |
| ATOM | 2634 | CB | THR | A | 331 | 8.685 | 79.154 | 5.400 | 1.00 | 46.65 |
| ATOM | 2635 | OG1 | THR | A | 331 | 8.904 | 77.740 | 5.517 | 1.00 | 38.61 |
| ATOM | 2636 | CG2 | THR | A | 331 | 7.378 | 79.483 | 6.144 | 1.00 | 48.69 |
| ATOM | 2637 | C | THR | A | 331 | 8.427 | 81.085 | 3.668 | 1.00 | 59.73 |
| ATOM | 2638 | O | THR | A | 331 | 8.586 | 81.496 | 2.495 | 1.00 | 55.52 |
| ATOM | 2639 | OT | THR | A | 331 | 8.131 | 81.869 | 4.601 | 1.00 | 62.34 |
| ATOM | 2640 | MN | MN | A | 350 | 10.357 | 71.058 | 3.078 | 1.00 | 32.10 |
| ATOM | 2641 | MN | MN | A | 351 | 16.765 | 98.946 | −5.069 | 1.00 | 40.69 |
| END | | | | | | | | | | |

TABLE 3

| | | | | | | |
|---|---|---|---|---|---|---|
| CRYST1 | 46.800 | 71.500 | 101.000 | 90.00 | 90.00 | 90.00 |

| SCALE1 | | 0.021368 | | 0.000000 | | 0.000000 | | 0.000000 | |
|---|---|---|---|---|---|---|---|---|---|
| SCALE2 | | 0.000000 | | 0.013986 | | 0.000000 | | 0.000000 | |
| SCALE3 | | 0.000000 | | 0.000000 | | 0.009901 | | 0.000000 | |
| ATOM | 1 | C1 | ACV | 1 | 17.235 | 36.323 | 5.699 | 1.00 | 7.93 |
| ATOM | 2 | C2 | ACV | 1 | 15.798 | 36.590 | 6.165 | 1.00 | 7.47 |
| ATOM | 3 | C3 | ACV | 1 | 15.215 | 37.802 | 5.425 | 1.00 | 6.42 |
| ATOM | 4 | C4 | ACV | 1 | 13.766 | 38.091 | 5.918 | 1.00 | 7.71 |
| ATOM | 5 | C7 | ACV | 1 | 13.330 | 39.380 | 5.168 | 1.00 | 8.43 |
| ATOM | 6 | C10 | ACV | 1 | 11.912 | 39.669 | 5.584 | 1.00 | 8.88 |
| ATOM | 7 | N11 | ACV | 1 | 10.931 | 39.447 | 4.714 | 1.00 | 6.98 |
| ATOM | 8 | C12 | ACV | 1 | 9.503 | 39.719 | 4.858 | 1.00 | 7.75 |
| ATOM | 9 | C13 | ACV | 1 | 8.767 | 38.397 | 4.657 | 1.00 | 7.09 |
| ATOM | 10 | N14 | ACV | 1 | 15.791 | 36.747 | 7.696 | 1.00 | 8.98 |
| ATOM | 11 | O15 | ACV | 1 | 11.566 | 40.061 | 6.715 | 1.00 | 11.68 |
| ATOM | 12 | C16 | ACV | 1 | 9.131 | 40.743 | 3.765 | 1.00 | 7.15 |
| ATOM | 13 | S17 | ACV | 1 | 9.513 | 40.068 | 2.102 | 1.00 | 8.44 |
| ATOM | 14 | O18 | ACV | 1 | 9.269 | 37.306 | 4.670 | 1.00 | 9.78 |
| ATOM | 15 | O19 | ACV | 1 | 18.173 | 36.442 | 6.549 | 1.00 | 8.96 |
| ATOM | 16 | O20 | ACV | 1 | 17.393 | 36.068 | 4.492 | 1.00 | 7.91 |
| ATOM | 17 | N29 | ACV | 1 | 7.424 | 38.510 | 4.604 | 1.00 | 9.16 |
| ATOM | 18 | C30 | ACV | 1 | 6.543 | 37.341 | 4.409 | 1.00 | 9.68 |
| ATOM | 19 | C31 | ACV | 1 | 5.317 | 37.433 | 5.318 | 1.00 | 10.48 |
| ATOM | 20 | C32 | ACV | 1 | 6.104 | 37.147 | 2.912 | 1.00 | 12.78 |
| ATOM | 21 | C33 | ACV | 1 | 7.348 | 36.829 | 2.039 | 1.00 | 11.31 |
| ATOM | 22 | C37 | ACV | 1 | 5.562 | 38.560 | 2.564 | 1.00 | 18.82 |
| ATOM | 23 | O42 | ACV | 1 | 5.240 | 38.298 | 6.210 | 1.00 | 10.58 |
| ATOM | 24 | O43 | ACV | 1 | 4.417 | 36.560 | 5.151 | 1.00 | 9.69 |
| ATOM | 25 | S | SUL | 2 | 13.002 | 14.100 | 2.417 | 1.00 | 25.69 |
| ATOM | 26 | O1 | SUL | 2 | 13.804 | 14.598 | 3.492 | 1.00 | 32.83 |
| ATOM | 27 | O2 | SUL | 2 | 13.918 | 13.558 | 1.424 | 1.00 | 41.91 |
| ATOM | 28 | O3 | SUL | 2 | 12.155 | 13.073 | 2.934 | 1.00 | 30.42 |
| ATOM | 29 | O4 | SUL | 2 | 12.299 | 15.076 | 1.614 | 1.00 | 21.23 |
| ATOM | 30 | FE | IUM | 1000 | 7.903 | 40.943 | 0.544 | 1.00 | 7.64 |
| ATOM | 31 | N | SER | 3 | −15.013 | 47.966 | −1.402 | 1.00 | 42.72 |
| ATOM | 32 | CA | SER | 3 | −14.317 | 46.679 | −1.445 | 1.00 | 39.06 |
| ATOM | 33 | C | SER | 3 | −12.942 | 46.953 | −2.052 | 1.00 | 36.17 |
| ATOM | 34 | O | SER | 3 | −12.712 | 48.077 | −2.493 | 1.00 | 41.73 |
| ATOM | 35 | CB | SER | 3 | −14.951 | 45.513 | −2.197 | 1.00 | 42.74 |
| ATOM | 36 | OG | SER | 3 | −14.920 | 45.578 | −3.613 | 1.00 | 52.50 |
| ATOM | 37 | N | VAL | 4 | −12.127 | 45.917 | −2.096 | 1.00 | 33.45 |
| ATOM | 38 | CA | VAL | 4 | −10.801 | 46.077 | −2.708 | 1.00 | 30.02 |
| ATOM | 39 | C | VAL | 4 | −10.826 | 45.243 | −3.983 | 1.00 | 26.11 |
| ATOM | 40 | O | VAL | 4 | −11.331 | 44.137 | −3.995 | 1.00 | 26.45 |
| ATOM | 41 | CB | VAL | 4 | −9.693 | 45.600 | −1.751 | 1.00 | 32.20 |
| ATOM | 42 | CG1 | VAL | 4 | −8.324 | 45.544 | −2.407 | 1.00 | 31.47 |
| ATOM | 43 | CG2 | VAL | 4 | −9.619 | 46.380 | −0.434 | 1.00 | 39.65 |
| ATOM | 44 | CB | SER | 5 | −9.685 | 46.084 | −7.342 | 1.00 | 28.73 |
| ATOM | 45 | OG | SER | 5 | −10.494 | 46.429 | −8.413 | 1.00 | 43.87 |
| ATOM | 46 | C | SER | 5 | −9.128 | 43.958 | −6.292 | 1.00 | 21.38 |
| ATOM | 47 | O | SER | 5 | −8.126 | 44.094 | −5.558 | 1.00 | 17.45 |
| ATOM | 48 | N | SER | 5 | −10.297 | 45.742 | −5.071 | 1.00 | 22.91 |
| ATOM | 49 | CA | SER | 5 | −10.216 | 45.050 | −6.347 | 1.00 | 24.13 |
| ATOM | 50 | N | LYS | 6 | −9.338 | 42.900 | −7.057 | 1.00 | 20.27 |
| ATOM | 51 | CA | LYS | 6 | −8.400 | 41.770 | −7.199 | 1.00 | 18.92 |
| ATOM | 52 | CB | LYS | 6 | −9.148 | 40.516 | −7.644 | 1.00 | 25.88 |
| ATOM | 53 | CG | LYS | 6 | −8.452 | 39.606 | −8.620 | 1.00 | 33.15 |
| ATOM | 54 | CD | LYS | 6 | −8.676 | 38.116 | −8.377 | 1.00 | 36.92 |
| ATOM | 55 | CE | LYS | 6 | −9.217 | 37.434 | −9.627 | 1.00 | 40.48 |
| ATOM | 56 | NZ | LYS | 6 | −10.331 | 38.278 | −10.180 | 1.00 | 49.46 |
| ATOM | 57 | C | LYS | 6 | −7.302 | 42.178 | −8.167 | 1.00 | 16.57 |
| ATOM | 58 | O | LYS | 6 | −7.476 | 42.719 | −9.294 | 1.00 | 19.33 |
| ATOM | 59 | N | ALA | 7 | −6.060 | 41.933 | −7.756 | 1.00 | 13.66 |
| ATOM | 60 | CA | ALA | 7 | −4.879 | 42.175 | −8.572 | 1.00 | 12.78 |
| ATOM | 61 | CB | ALA | 7 | −3.616 | 42.083 | −7.716 | 1.00 | 14.08 |
| ATOM | 62 | C | ALA | 7 | −4.803 | 41.135 | −9.678 | 1.00 | 12.30 |
| ATOM | 63 | O | ALA | 7 | −5.069 | 39.957 | −9.497 | 1.00 | 13.11 |
| ATOM | 64 | N | ASN | 8 | −4.325 | 41.585 | −10.844 | 1.00 | 15.13 |
| ATOM | 65 | CA | ASN | 8 | −4.026 | 40.653 | −11.913 | 1.00 | 16.54 |
| ATOM | 66 | CB | ASN | 8 | −3.650 | 41.448 | −13.197 | 1.00 | 24.27 |
| ATOM | 67 | CG | ASN | 8 | −4.274 | 40.597 | −14.298 | 1.00 | 29.61 |
| ATOM | 68 | OD1 | ASN | 8 | −3.669 | 39.640 | −14.787 | 1.00 | 35.60 |
| ATOM | 69 | ND2 | ASN | 8 | −5.528 | 40.986 | −14.477 | 1.00 | 43.75 |
| ATOM | 70 | C | ASN | 8 | −2.738 | 39.882 | −11.623 | 1.00 | 13.63 |
| ATOM | 71 | O | ASN | 8 | −1.648 | 40.451 | −11.691 | 1.00 | 16.79 |
| ATOM | 72 | N | VAL | 9 | −2.918 | 38.611 | −11.303 | 1.00 | 11.43 |
| ATOM | 73 | CA | VAL | 9 | −1.809 | 37.707 | −11.016 | 1.00 | 10.00 |
| ATOM | 74 | CB | VAL | 9 | −1.770 | 37.383 | −9.522 | 1.00 | 10.55 |
| ATOM | 75 | CG1 | VAL | 9 | −0.548 | 36.537 | −9.229 | 1.00 | 10.77 |
| ATOM | 76 | CG2 | VAL | 9 | −1.726 | 38.675 | −8.725 | 1.00 | 11.32 |
| ATOM | 77 | C | VAL | 9 | −1.955 | 36.422 | −11.845 | 1.00 | 10.66 |
| ATOM | 78 | O | VAL | 9 | −2.621 | 35.466 | −11.464 | 1.00 | 14.19 |
| ATOM | 79 | N | PRO | 10 | −1.385 | 36.425 | −13.059 | 1.00 | 9.44 |
| ATOM | 80 | CD | PRO | 10 | −0.544 | 37.474 | −13.650 | 1.00 | 10.90 |
| ATOM | 81 | CA | PRO | 10 | −1.565 | 35.299 | −13.942 | 1.00 | 10.48 |
| ATOM | 82 | CB | PRO | 10 | −0.901 | 35.749 | −15.235 | 1.00 | 12.92 |
| ATOM | 83 | CG | PRO | 10 | −0.067 | 36.924 | −14.937 | 1.00 | 15.89 |
| ATOM | 84 | C | PRO | 10 | −0.883 | 34.026 | −13.444 | 1.00 | 9.89 |
| ATOM | 85 | O | PRO | 10 | 0.125 | 34.091 | −12.734 | 1.00 | 10.22 |
| ATOM | 86 | N | LYS | 11 | −1.414 | 32.896 | −13.847 | 1.00 | 9.52 |
| ATOM | 87 | CA | LYS | 11 | −0.815 | 31.597 | −13.586 | 1.00 | 9.53 |
| ATOM | 88 | CB | LYS | 11 | −1.885 | 30.560 | −13.230 | 1.00 | 12.58 |
| ATOM | 89 | CG | LYS | 11 | −2.651 | 30.971 | −11.965 | 1.00 | 18.45 |
| ATOM | 90 | CD | LYS | 11 | −3.746 | 30.048 | −11.504 | 1.00 | 23.78 |
| ATOM | 91 | CE | LYS | 11 | −4.685 | 30.872 | −10.629 | 1.00 | 25.46 |
| ATOM | 92 | NZ | LYS | 11 | −4.154 | 31.101 | −9.250 | 1.00 | 27.77 |
| ATOM | 93 | C | LYS | 11 | 0.020 | 31.211 | −14.803 | 1.00 | 10.29 |
| ATOM | 94 | O | LYS | 11 | −0.482 | 31.172 | −15.926 | 1.00 | 16.95 |
| ATOM | 95 | N | ILE | 12 | 1.301 | 31.019 | −14.640 | 1.00 | 8.18 |
| ATOM | 96 | CA | ILE | 12 | 2.214 | 30.697 | −15.742 | 1.00 | 8.29 |
| ATOM | 97 | CB | ILE | 12 | 3.358 | 31.733 | −15.815 | 1.00 | 8.64 |
| ATOM | 98 | CG2 | ILE | 12 | 4.366 | 31.311 | −16.864 | 1.00 | 9.33 |
| ATOM | 99 | CG1 | ILE | 12 | 2.860 | 33.160 | −16.018 | 1.00 | 9.90 |
| ATOM | 100 | CD1 | ILE | 12 | 3.945 | 34.238 | −15.984 | 1.00 | 10.28 |
| ATOM | 101 | C | ILE | 12 | 2.749 | 29.284 | −15.518 | 1.00 | 7.89 |
| ATOM | 102 | O | ILE | 12 | 3.346 | 28.974 | −14.504 | 1.00 | 7.80 |
| ATOM | 103 | N | ASP | 13 | 2.542 | 28.428 | −16.522 | 1.00 | 8.72 |
| ATOM | 104 | CA | ASP | 13 | 3.109 | 27.068 | −16.533 | 1.00 | 8.16 |
| ATOM | 105 | CB | ASP | 13 | 2.391 | 26.193 | −17.536 | 1.00 | 9.68 |
| ATOM | 106 | CG | ASP | 13 | 2.947 | 24.828 | −17.728 | 1.00 | 11.65 |
| ATOM | 107 | OD1 | ASP | 13 | 4.047 | 24.478 | −17.257 | 1.00 | 10.88 |
| ATOM | 108 | OD2 | ASP | 13 | 2.283 | 24.013 | −18.401 | 1.00 | 19.22 |
| ATOM | 109 | C | ASP | 13 | 4.601 | 27.248 | −16.838 | 1.00 | 7.72 |
| ATOM | 110 | O | ASP | 13 | 5.005 | 27.527 | −17.990 | 1.00 | 9.02 |
| ATOM | 111 | N | VAL | 14 | 5.413 | 26.983 | −15.825 | 1.00 | 8.27 |
| ATOM | 112 | CA | VAL | 14 | 6.862 | 27.170 | −15.947 | 1.00 | 8.27 |
| ATOM | 113 | CB | VAL | 14 | 7.453 | 27.766 | −14.680 | 1.00 | 8.09 |
| ATOM | 114 | CG1 | VAL | 14 | 6.890 | 29.164 | −14.465 | 1.00 | 9.81 |
| ATOM | 115 | CG2 | VAL | 14 | 7.298 | 26.882 | −13.458 | 1.00 | 8.58 |
| ATOM | 116 | C | VAL | 14 | 7.592 | 25.910 | −16.328 | 1.00 | 8.98 |
| ATOM | 117 | O | VAL | 14 | 8.815 | 25.950 | −16.464 | 1.00 | 10.04 |
| ATOM | 118 | N | SER | 15 | 6.851 | 24.822 | −16.531 | 1.00 | 9.65 |
| ATOM | 119 | CA | SER | 15 | 7.532 | 23.572 | −16.883 | 1.00 | 9.94 |
| ATOM | 120 | CB | SER | 15 | 6.548 | 22.411 | −16.994 | 1.00 | 10.80 |
| ATOM | 121 | OG | SER | 15 | 5.618 | 22.532 | −18.063 | 1.00 | 12.34 |
| ATOM | 122 | C | SER | 15 | 8.469 | 23.599 | −18.070 | 1.00 | 9.33 |
| ATOM | 123 | O | SER | 15 | 9.519 | 22.915 | −18.009 | 1.00 | 9.96 |
| ATOM | 124 | N | PRO | 16 | 8.218 | 24.364 | −19.141 | 1.00 | 10.18 |
| ATOM | 125 | CD | PRO | 16 | 7.026 | 25.108 | −19.546 | 1.00 | 9.79 |
| ATOM | 126 | CA | PRO | 16 | 9.220 | 24.381 | −20.209 | 1.00 | 10.12 |
| ATOM | 127 | CB | PRO | 16 | 8.629 | 25.357 | −21.226 | 1.00 | 10.60 |
| ATOM | 128 | CG | PRO | 16 | 7.127 | 25.247 | −21.015 | 1.00 | 11.12 |
| ATOM | 129 | C | PRO | 16 | 10.583 | 24.909 | −19.807 | 1.00 | 9.66 |
| ATOM | 130 | O | PRO | 16 | 11.579 | 24.613 | −20.444 | 1.00 | 11.72 |
| ATOM | 131 | N | LEU | 17 | 10.666 | 25.693 | −18.711 | 1.00 | 10.05 |
| ATOM | 132 | CA | LEU | 17 | 11.949 | 26.232 | −18.288 | 1.00 | 10.06 |
| ATOM | 133 | CB | LEU | 17 | 11.738 | 27.358 | −17.276 | 1.00 | 8.52 |
| ATOM | 134 | CG | LEU | 17 | 10.992 | 28.598 | −17.808 | 1.00 | 9.04 |
| ATOM | 135 | CD1 | LEU | 17 | 10.784 | 29.540 | −16.622 | 1.00 | 9.12 |
| ATOM | 136 | CD2 | LEU | 17 | 11.738 | 29.314 | −18.922 | 1.00 | 10.89 |
| ATOM | 137 | C | LEU | 17 | 12.890 | 25.183 | −17.692 | 1.00 | 11.62 |
| ATOM | 138 | O | LEU | 17 | 14.087 | 25.442 | −17.466 | 1.00 | 12.52 |
| ATOM | 139 | N | PHE | 18 | 12.403 | 23.970 | −17.499 | 1.00 | 11.77 |
| ATOM | 140 | CA | PHE | 18 | 13.234 | 22.862 | −17.065 | 1.00 | 12.92 |
| ATOM | 141 | CB | PHE | 18 | 12.363 | 21.947 | −16.180 | 1.00 | 12.94 |
| ATOM | 142 | CG | PHE | 18 | 12.070 | 22.571 | −14.820 | 1.00 | 13.53 |
| ATOM | 143 | CD1 | PHE | 18 | 10.872 | 23.207 | −14.599 | 1.00 | 17.84 |
| ATOM | 144 | CD2 | PHE | 18 | 12.965 | 22.503 | −13.766 | 1.00 | 14.00 |
| ATOM | 145 | CE1 | PHE | 18 | 10.537 | 23.720 | −13.359 | 1.00 | 17.86 |
| ATOM | 146 | CE2 | PHE | 18 | 12.638 | 22.974 | −12.519 | 1.00 | 14.38 |
| ATOM | 147 | CZ | PHE | 18 | 11.444 | 23.614 | −12.326 | 1.00 | 15.20 |
| ATOM | 148 | C | PHE | 18 | 13.768 | 22.054 | −18.231 | 1.00 | 14.72 |
| ATOM | 149 | O | PHE | 18 | 14.567 | 21.129 | −18.012 | 1.00 | 17.55 |
| ATOM | 150 | N | GLY | 19 | 13.321 | 22.349 | −19.445 | 1.00 | 15.60 |
| ATOM | 151 | CA | GLY | 19 | 13.718 | 21.513 | −20.583 | 1.00 | 17.05 |
| ATOM | 152 | C | GLY | 19 | 14.489 | 22.248 | −21.663 | 1.00 | 16.88 |
| ATOM | 153 | O | GLY | 19 | 15.092 | 23.280 | −21.384 | 1.00 | 16.84 |
| ATOM | 154 | N | ASP | 20 | 14.471 | 21.679 | −22.868 | 1.00 | 18.23 |
| ATOM | 155 | CA | ASP | 20 | 15.241 | 22.147 | −23.997 | 1.00 | 19.57 |
| ATOM | 156 | C | ASP | 20 | 14.418 | 22.595 | −25.186 | 1.00 | 16.96 |
| ATOM | 157 | O | ASP | 20 | 14.976 | 22.646 | −26.285 | 1.00 | 18.78 |
| ATOM | 158 | CB | ASP | 20 | 16.172 | 21.025 | −24.491 | 1.00 | 25.96 |
| ATOM | 159 | CG | ASP | 20 | 16.954 | 20.446 | −23.320 | 1.00 | 30.78 |
| ATOM | 160 | OD1 | ASP | 20 | 17.102 | 19.208 | −23.399 | 1.00 | 38.92 |
| ATOM | 161 | OD2 | ASP | 20 | 17.315 | 21.213 | −22.393 | 1.00 | 40.12 |
| ATOM | 162 | N | ASP | 21 | 13.161 | 22.913 | −25.013 | 1.00 | 16.58 |
| ATOM | 163 | CA | ASP | 21 | 12.383 | 23.435 | −26.145 | 1.00 | 16.91 |
| ATOM | 164 | CB | ASP | 21 | 10.920 | 23.028 | −25.985 | 1.00 | 18.23 |
| ATOM | 165 | CG | ASP | 21 | 10.023 | 23.362 | −27.142 | 1.00 | 20.86 |
| ATOM | 166 | OD1 | ASP | 21 | 10.313 | 24.380 | −27.796 | 1.00 | 21.70 |
| ATOM | 167 | OD2 | ASP | 21 | 8.968 | 22.756 | −27.430 | 1.00 | 27.79 |
| ATOM | 168 | C | ASP | 21 | 12.561 | 24.961 | −26.116 | 1.00 | 14.20 |
| ATOM | 169 | O | ASP | 21 | 11.892 | 25.648 | −25.343 | 1.00 | 12.84 |
| ATOM | 170 | N | GLN | 22 | 13.504 | 25.500 | −26.887 | 1.00 | 15.68 |
| ATOM | 171 | CA | GLN | 22 | 13.813 | 26.915 | −26.764 | 1.00 | 13.82 |
| ATOM | 172 | CB | GLN | 22 | 15.048 | 27.347 | −27.545 | 1.00 | 13.58 |
| ATOM | 173 | CG | GLN | 22 | 16.257 | 26.474 | −27.348 | 1.00 | 15.37 |
| ATOM | 174 | CD | GLN | 22 | 16.663 | 26.189 | −25.918 | 1.00 | 16.56 |
| ATOM | 175 | OE1 | GLN | 22 | 16.485 | 27.042 | −25.067 | 1.00 | 19.01 |
| ATOM | 176 | NE2 | GLN | 22 | 17.205 | 24.984 | −25.728 | 1.00 | 19.39 |
| ATOM | 177 | C | GLN | 22 | 12.654 | 27.819 | −27.116 | 1.00 | 13.52 |
| ATOM | 178 | O | GLN | 22 | 12.484 | 28.853 | −26.449 | 1.00 | 11.96 |
| ATOM | 179 | N | ALA | 23 | 11.890 | 27.480 | −28.125 | 1.00 | 16.53 |
| ATOM | 180 | CA | ALA | 23 | 10.739 | 28.320 | −28.488 | 1.00 | 16.41 |
| ATOM | 181 | CB | ALA | 23 | 10.088 | 27.855 | −29.796 | 1.00 | 22.56 |
| ATOM | 182 | C | ALA | 23 | 9.715 | 28.331 | −27.352 | 1.00 | 13.99 |
| ATOM | 183 | O | ALA | 23 | 9.120 | 29.380 | −27.065 | 1.00 | 13.68 |
| ATOM | 184 | N | ALA | 24 | 9.451 | 27.160 | −26.761 | 1.00 | 13.08 |
| ATOM | 185 | CA | ALA | 24 | 8.481 | 27.122 | −25.650 | 1.00 | 11.55 |
| ATOM | 186 | CB | ALA | 24 | 8.214 | 25.694 | −25.217 | 1.00 | 13.62 |
| ATOM | 187 | C | ALA | 24 | 8.988 | 27.977 | −24.524 | 1.00 | 9.62 |
| ATOM | 188 | O | ALA | 24 | 8.213 | 28.627 | −23.815 | 1.00 | 9.80 |
| ATOM | 189 | N | LYS | 25 | 10.278 | 27.958 | −24.263 | 1.00 | 10.01 |
| ATOM | 190 | CA | LYS | 25 | 10.844 | 28.781 | −23.178 | 1.00 | 9.16 |
| ATOM | 191 | CB | LYS | 25 | 12.332 | 28.472 | −23.004 | 1.00 | 9.87 |
| ATOM | 192 | CG | LYS | 25 | 12.600 | 27.128 | −22.327 | 1.00 | 13.94 |
| ATOM | 193 | CD | LYS | 25 | 14.077 | 27.106 | −21.852 | 1.00 | 21.25 |
| ATOM | 194 | CE | LYS | 25 | 14.817 | 25.974 | −22.406 | 1.00 | 24.07 |
| ATOM | 195 | NZ | LYS | 25 | 16.254 | 26.073 | −22.150 | 1.00 | 19.05 |
| ATOM | 196 | C | LYS | 25 | 10.657 | 30.249 | −23.474 | 1.00 | 7.92 |
| ATOM | 197 | O | LYS | 25 | 10.375 | 31.042 | −22.566 | 1.00 | 8.19 |
| ATOM | 198 | N | MET | 26 | 10.811 | 30.662 | −24.728 | 1.00 | 8.75 |
| ATOM | 199 | CA | MET | 26 | 10.564 | 32.085 | −25.068 | 1.00 | 9.12 |
| ATOM | 200 | CB | MET | 26 | 10.903 | 32.408 | −26.546 | 1.00 | 9.73 |
| ATOM | 201 | CG | MET | 26 | 12.399 | 32.424 | −26.816 | 1.00 | 10.03 |
| ATOM | 202 | SD | MET | 26 | 13.322 | 33.724 | −25.970 | 1.00 | 10.21 |
| ATOM | 203 | CE | MET | 26 | 13.056 | 35.132 | −27.066 | 1.00 | 10.95 |
| ATOM | 204 | C | MET | 26 | 9.115 | 32.487 | −24.804 | 1.00 | 8.72 |
| ATOM | 205 | O | MET | 26 | 8.828 | 33.594 | −24.356 | 1.00 | 8.27 |
| ATOM | 206 | N | ARG | 27 | 8.163 | 31.585 | −25.091 | 1.00 | 8.42 |
| ATOM | 207 | CA | ARG | 27 | 6.767 | 31.903 | −24.809 | 1.00 | 9.14 |
| ATOM | 208 | CB | ARG | 27 | 5.842 | 30.894 | −25.505 | 1.00 | 10.27 |
| ATOM | 209 | CG | ARG | 27 | 5.895 | 31.042 | −27.031 | 1.00 | 11.63 |
| ATOM | 210 | CD | ARG | 27 | 4.969 | 30.149 | −27.808 | 1.00 | 15.41 |
| ATOM | 211 | NE | ARG | 27 | 5.322 | 28.732 | −27.657 | 1.00 | 17.68 |
| ATOM | 212 | CZ | ARG | 27 | 5.998 | 28.016 | −28.551 | 1.00 | 17.12 |
| ATOM | 213 | NH1 | ARG | 27 | 6.271 | 26.720 | −28.347 | 1.00 | 18.08 |
| ATOM | 214 | NH2 | ARG | 27 | 6.357 | 28.591 | −29.680 | 1.00 | 19.32 |
| ATOM | 215 | C | ARG | 27 | 6.496 | 32.020 | −23.319 | 1.00 | 9.48 |
| ATOM | 216 | O | ARG | 27 | 5.649 | 32.854 | −22.925 | 1.00 | 9.79 |
| ATOM | 217 | N | VAL | 28 | 7.214 | 31.249 | −22.488 | 1.00 | 7.80 |
| ATOM | 218 | CA | VAL | 28 | 7.129 | 31.447 | −21.039 | 1.00 | 7.80 |
| ATOM | 219 | CB | VAL | 28 | 7.799 | 30.307 | −20.275 | 1.00 | 8.02 |
| ATOM | 220 | CG1 | VAL | 28 | 7.803 | 30.582 | −18.782 | 1.00 | 9.37 |
| ATOM | 221 | CG2 | VAL | 28 | 7.184 | 28.970 | −20.553 | 1.00 | 10.98 |
| ATOM | 222 | C | VAL | 28 | 7.743 | 32.786 | −20.643 | 1.00 | 7.31 |
| ATOM | 223 | O | VAL | 28 | 7.164 | 33.556 | −19.853 | 1.00 | 7.76 |
| ATOM | 224 | N | ALA | 29 | 8.909 | 33.096 | −21.235 | 1.00 | 7.04 |
| ATOM | 225 | CA | ALA | 29 | 9.578 | 34.368 | −20.949 | 1.00 | 7.37 |
| ATOM | 226 | CB | ALA | 29 | 10.870 | 34.447 | −21.743 | 1.00 | 7.98 |
| ATOM | 227 | C | ALA | 29 | 8.691 | 35.579 | −21.233 | 1.00 | 7.31 |
| ATOM | 228 | O | ALA | 29 | 8.662 | 36.547 | −20.478 | 1.00 | 8.13 |
| ATOM | 229 | N | GLN | 30 | 7.902 | 35.483 | −22.319 | 1.00 | 7.11 |
| ATOM | 230 | CA | GLN | 30 | 7.001 | 36.576 | −22.637 | 1.00 | 7.56 |
| ATOM | 231 | CB | GLN | 30 | 6.261 | 36.323 | −23.976 | 1.00 | 8.19 |
| ATOM | 232 | CG | GLN | 30 | 5.378 | 37.456 | −24.427 | 1.00 | 8.58 |
| ATOM | 233 | CD | GLN | 30 | 3.966 | 37.535 | −23.845 | 1.00 | 8.95 |
| ATOM | 234 | OE1 | GLN | 30 | 3.396 | 36.491 | −23.534 | 1.00 | 10.78 |
| ATOM | 235 | NE2 | GLN | 30 | 3.570 | 38.741 | −23.481 | 1.00 | 9.99 |
| ATOM | 236 | C | GLN | 30 | 6.005 | 36.784 | −21.497 | 1.00 | 6.94 |
| ATOM | 237 | O | GLN | 30 | 5.631 | 37.936 | −21.231 | 1.00 | 8.27 |
| ATOM | 238 | N | GLN | 31 | 5.464 | 35.677 | −20.996 | 1.00 | 7.41 |
| ATOM | 239 | CA | GLN | 31 | 4.470 | 35.750 | −19.933 | 1.00 | 7.30 |
| ATOM | 240 | CB | GLN | 31 | 3.924 | 34.357 | −19.675 | 1.00 | 8.13 |
| ATOM | 241 | CG | GLN | 31 | 3.117 | 33.748 | −20.829 | 1.00 | 9.37 |
| ATOM | 242 | CD | GLN | 31 | 2.722 | 32.316 | −20.493 | 1.00 | 11.83 |
| ATOM | 243 | OE1 | GLN | 31 | 1.843 | 32.090 | −19.656 | 1.00 | 15.21 |
| ATOM | 244 | NE2 | GLN | 31 | 3.391 | 31.367 | −21.108 | 1.00 | 18.42 |
| ATOM | 245 | C | GLN | 31 | 5.093 | 36.370 | −18.681 | 1.00 | 7.43 |
| ATOM | 246 | O | GLN | 31 | 4.459 | 37.197 | −18.024 | 1.00 | 7.98 |
| ATOM | 247 | N | ILE | 32 | 6.326 | 36.030 | −18.367 | 1.00 | 6.96 |
| ATOM | 248 | CA | ILE | 32 | 7.047 | 36.640 | −17.258 | 1.00 | 7.29 |
| ATOM | 249 | CB | ILE | 32 | 8.389 | 35.925 | −17.010 | 1.00 | 7.37 |
| ATOM | 250 | CG2 | ILE | 32 | 9.254 | 36.685 | −16.004 | 1.00 | 9.11 |
| ATOM | 251 | CG1 | ILE | 32 | 8.126 | 34.504 | −16.497 | 1.00 | 7.84 |
| ATOM | 252 | CD1 | ILE | 32 | 9.335 | 33.611 | −16.390 | 1.00 | 9.10 |
| ATOM | 253 | C | ILE | 32 | 7.235 | 38.139 | −17.496 | 1.00 | 7.13 |
| ATOM | 254 | O | ILE | 32 | 7.023 | 38.945 | −16.602 | 1.00 | 8.31 |
| ATOM | 255 | N | ASP | 33 | 7.632 | 38.515 | −18.717 | 1.00 | 7.76 |
| ATOM | 256 | CA | ASP | 33 | 7.801 | 39.927 | −19.072 | 1.00 | 8.04 |
| ATOM | 257 | CB | ASP | 33 | 8.257 | 40.026 | −20.550 | 1.00 | 8.19 |
| ATOM | 258 | CG | ASP | 33 | 8.447 | 41.482 | −20.994 | 1.00 | 8.88 |
| ATOM | 259 | OD1 | ASP | 33 | 9.429 | 42.118 | −20.550 | 1.00 | 9.63 |
| ATOM | 260 | OD2 | ASP | 33 | 7.560 | 41.941 | −21.792 | 1.00 | 9.07 |
| ATOM | 261 | C | ASP | 33 | 6.480 | 40.675 | −18.835 | 1.00 | 7.95 |
| ATOM | 262 | O | ASP | 33 | 6.480 | 41.751 | −18.200 | 1.00 | 8.54 |
| ATOM | 263 | N | ALA | 34 | 5.357 | 40.154 | −19.355 | 1.00 | 8.10 |
| ATOM | 264 | CA | ALA | 34 | 4.079 | 40.834 | −19.202 | 1.00 | 8.69 |
| ATOM | 265 | CB | ALA | 34 | 2.993 | 40.062 | −19.938 | 1.00 | 9.24 |
| ATOM | 266 | C | ALA | 34 | 3.709 | 41.028 | −17.735 | 1.00 | 8.36 |
| ATOM | 267 | O | ALA | 34 | 3.284 | 42.105 | −17.299 | 1.00 | 9.82 |
| ATOM | 268 | N | ALA | 35 | 3.903 | 39.967 | −16.933 | 1.00 | 8.64 |
| ATOM | 269 | CA | ALA | 35 | 3.505 | 40.093 | −15.538 | 1.00 | 9.10 |
| ATOM | 270 | CB | ALA | 35 | 3.527 | 38.723 | −14.870 | 1.00 | 11.05 |
| ATOM | 271 | C | ALA | 35 | 4.423 | 41.047 | −14.779 | 1.00 | 9.17 |
| ATOM | 272 | O | ALA | 35 | 3.968 | 41.822 | −13.942 | 1.00 | 10.62 |
| ATOM | 273 | N | SER | 36 | 5.709 | 41.042 | −15.146 | 1.00 | 8.20 |
| ATOM | 274 | CA | SER | 36 | 6.683 | 41.896 | −14.467 | 1.00 | 7.99 |
| ATOM | 275 | CB | SER | 36 | 8.108 | 41.485 | −14.830 | 1.00 | 8.90 |
| ATOM | 276 | OG | SER | 36 | 8.354 | 40.129 | −14.485 | 1.00 | 8.75 |
| ATOM | 277 | C | SER | 36 | 6.436 | 43.364 | −14.801 | 1.00 | 9.43 |
| ATOM | 278 | O | SER | 36 | 6.761 | 44.224 | −13.994 | 1.00 | 12.45 |
| ATOM | 279 | N | ARG | 37 | 5.871 | 43.633 | −15.993 | 1.00 | 9.00 |
| ATOM | 280 | CA | ARG | 37 | 5.572 | 44.996 | −16.415 | 1.00 | 10.53 |
| ATOM | 281 | CB | ARG | 37 | 5.685 | 45.095 | −17.931 | 1.00 | 11.22 |
| ATOM | 282 | CG | ARG | 37 | 7.046 | 44.858 | −18.544 | 1.00 | 12.00 |
| ATOM | 283 | CD | ARG | 37 | 7.074 | 44.615 | −20.051 | 1.00 | 14.37 |
| ATOM | 284 | NE | ARG | 37 | 6.514 | 45.706 | −20.881 | 1.00 | 15.54 |
| ATOM | 285 | CZ | ARG | 37 | 6.327 | 45.575 | −22.190 | 1.00 | 15.00 |
| ATOM | 286 | NH1 | ARG | 37 | 6.682 | 44.430 | −22.781 | 1.00 | 16.31 |
| ATOM | 287 | NH2 | ARG | 37 | 5.812 | 46.595 | −22.875 | 1.00 | 14.87 |
| ATOM | 288 | C | ARG | 37 | 4.204 | 45.459 | −15.925 | 1.00 | 11.82 |
| ATOM | 289 | O | ARG | 37 | 3.914 | 46.654 | −15.947 | 1.00 | 17.60 |
| ATOM | 290 | N | ASP | 38 | 3.367 | 44.520 | −15.475 | 1.00 | 12.19 |
| ATOM | 291 | CA | ASP | 38 | 2.045 | 44.889 | −14.976 | 1.00 | 14.66 |
| ATOM | 292 | CB | ASP | 38 | 1.017 | 43.860 | −15.463 | 1.00 | 16.65 |
| ATOM | 293 | CG | ASP | 38 | −0.441 | 44.105 | −15.173 | 1.00 | 17.06 |
| ATOM | 294 | OD1 | ASP | 38 | −0.763 | 45.278 | −14.922 | 1.00 | 22.39 |
| ATOM | 295 | OD2 | ASP | 38 | −1.316 | 43.219 | −15.263 | 1.00 | 20.09 |
| ATOM | 296 | C | ASP | 38 | 2.102 | 45.018 | −13.455 | 1.00 | 13.71 |
| ATOM | 297 | O | ASP | 38 | 2.736 | 45.927 | −12.904 | 1.00 | 14.27 |
| ATOM | 298 | N | THR | 39 | 1.460 | 44.073 | −12.737 | 1.00 | 11.56 |
| ATOM | 299 | CA | THR | 39 | 1.415 | 44.205 | −11.298 | 1.00 | 11.81 |
| ATOM | 300 | CB | THR | 39 | 0.320 | 43.390 | −10.584 | 1.00 | 14.06 |
| ATOM | 301 | OG1 | THR | 39 | 0.687 | 41.990 | −10.698 | 1.00 | 17.03 |
| ATOM | 302 | CG2 | THR | 39 | −1.008 | 43.691 | −11.223 | 1.00 | 17.60 |
| ATOM | 303 | C | THR | 39 | 2.721 | 43.776 | −10.634 | 1.00 | 10.17 |
| ATOM | 304 | O | THR | 39 | 2.920 | 44.118 | −9.459 | 1.00 | 11.25 |
| ATOM | 305 | N | GLY | 40 | 3.510 | 42.982 | −11.367 | 1.00 | 9.14 |
| ATOM | 306 | CA | GLY | 40 | 4.740 | 42.526 | −10.777 | 1.00 | 8.52 |
| ATOM | 307 | C | GLY | 40 | 4.618 | 41.178 | −10.115 | 1.00 | 9.63 |
| ATOM | 308 | O | GLY | 40 | 5.587 | 40.753 | −9.505 | 1.00 | 14.89 |
| ATOM | 309 | N | PHE | 41 | 3.477 | 40.522 | −10.124 | 1.00 | 8.18 |
| ATOM | 310 | CA | PHE | 41 | 3.307 | 39.207 | −9.532 | 1.00 | 7.48 |
| ATOM | 311 | CB | PHE | 41 | 2.353 | 39.290 | −8.343 | 1.00 | 7.50 |
| ATOM | 312 | CG | PHE | 41 | 2.952 | 39.927 | −7.078 | 1.00 | 7.93 |
| ATOM | 313 | CD1 | PHE | 41 | 2.936 | 41.286 | −6.859 | 1.00 | 8.43 |
| ATOM | 314 | CD2 | PHE | 41 | 3.499 | 39.110 | −6.100 | 1.00 | 7.47 |
| ATOM | 315 | CE1 | PHE | 41 | 3.446 | 41.812 | −5.683 | 1.00 | 8.79 |
| ATOM | 316 | CE2 | PHE | 41 | 4.040 | 39.635 | −4.933 | 1.00 | 8.21 |
| ATOM | 317 | CZ | PHE | 41 | 4.032 | 40.998 | −4.757 | 1.00 | 8.46 |
| ATOM | 318 | C | PHE | 41 | 2.782 | 38.226 | −10.550 | 1.00 | 6.88 |
| ATOM | 319 | O | PHE | 41 | 1.952 | 38.554 | −11.401 | 1.00 | 9.03 |
| ATOM | 320 | N | PHE | 42 | 3.164 | 36.978 | −10.374 | 1.00 | 6.68 |
| ATOM | 321 | CA | PHE | 42 | 2.539 | 35.845 | −11.075 | 1.00 | 6.92 |
| ATOM | 322 | CB | PHE | 42 | 3.148 | 35.586 | −12.456 | 1.00 | 7.96 |
| ATOM | 323 | CG | PHE | 42 | 4.564 | 35.072 | −12.516 | 1.00 | 7.35 |
| ATOM | 324 | CD1 | PHE | 42 | 4.802 | 33.699 | −12.586 | 1.00 | 7.79 |
| ATOM | 325 | CD2 | PHE | 42 | 5.637 | 35.931 | −12.518 | 1.00 | 8.85 |
| ATOM | 326 | CE1 | PHE | 42 | 6.124 | 33.266 | −12.696 | 1.00 | 8.64 |
| ATOM | 327 | CE2 | PHE | 42 | 6.951 | 35.498 | −12.656 | 1.00 | 9.37 |
| ATOM | 328 | CZ | PHE | 42 | 7.193 | 34.141 | −12.740 | 1.00 | 9.28 |
| ATOM | 329 | C | PHE | 42 | 2.620 | 34.594 | −10.216 | 1.00 | 6.60 |
| ATOM | 330 | O | PHE | 42 | 3.489 | 34.518 | −9.338 | 1.00 | 6.98 |
| ATOM | 331 | N | TYR | 43 | 1.783 | 33.606 | −10.477 | 1.00 | 6.57 |
| ATOM | 332 | CA | TYR | 43 | 1.913 | 32.290 | −9.860 | 1.00 | 6.96 |
| ATOM | 333 | CB | TYR | 43 | 0.575 | 31.694 | −9.466 | 1.00 | 7.72 |
| ATOM | 334 | CG | TYR | 43 | 0.098 | 32.111 | −8.088 | 1.00 | 7.41 |
| ATOM | 335 | CD1 | TYR | 43 | −0.901 | 33.078 | −7.938 | 1.00 | 8.92 |
| ATOM | 336 | CE1 | TYR | 43 | −1.335 | 33.480 | −6.697 | 1.00 | 9.19 |
| ATOM | 337 | CD2 | TYR | 43 | 0.664 | 31.618 | −6.939 | 1.00 | 7.76 |
| ATOM | 338 | CE2 | TYR | 43 | 0.248 | 32.035 | −5.690 | 1.00 | 8.82 |
| ATOM | 339 | CZ | TYR | 43 | −0.715 | 32.992 | −5.574 | 1.00 | 9.99 |
| ATOM | 340 | OH | TYR | 43 | −1.130 | 33.349 | −4.303 | 1.00 | 11.00 |
| ATOM | 341 | C | TYR | 43 | 2.625 | 31.390 | −10.867 | 1.00 | 6.77 |
| ATOM | 342 | O | TYR | 43 | 2.160 | 31.203 | −11.985 | 1.00 | 9.17 |
| ATOM | 343 | N | ALA | 44 | 3.663 | 30.736 | −10.386 | 1.00 | 6.74 |
| ATOM | 344 | CA | ALA | 44 | 4.321 | 29.654 | −11.138 | 1.00 | 6.80 |
| ATOM | 345 | CB | ALA | 44 | 5.766 | 29.522 | −10.683 | 1.00 | 6.89 |
| ATOM | 346 | C | ALA | 44 | 3.590 | 28.345 | −10.834 | 1.00 | 6.81 |
| ATOM | 347 | O | ALA | 44 | 3.423 | 27.965 | −9.679 | 1.00 | 7.69 |
| ATOM | 348 | N | VAL | 45 | 3.075 | 27.695 | −11.849 | 1.00 | 7.45 |
| ATOM | 349 | CA | VAL | 45 | 2.373 | 26.431 | −11.766 | 1.00 | 7.41 |
| ATOM | 350 | CB | VAL | 45 | 0.902 | 26.570 | −12.161 | 1.00 | 9.68 |
| ATOM | 351 | CG1 | VAL | 45 | 0.228 | 27.601 | −11.255 | 1.00 | 10.94 |
| ATOM | 352 | CG2 | VAL | 45 | 0.670 | 26.895 | −13.632 | 1.00 | 10.29 |
| ATOM | 353 | C | VAL | 45 | 3.120 | 25.384 | −12.583 | 1.00 | 7.22 |
| ATOM | 354 | O | VAL | 45 | 3.984 | 25.718 | −13.393 | 1.00 | 7.51 |
| ATOM | 355 | N | ASN | 46 | 2.862 | 24.095 | −12.325 | 1.00 | 8.30 |
| ATOM | 356 | CA | ASN | 46 | 3.565 | 22.985 | −12.948 | 1.00 | 8.32 |
| ATOM | 357 | CB | ASN | 46 | 3.323 | 22.885 | −14.449 | 1.00 | 11.81 |
| ATOM | 358 | CG | ASN | 46 | 1.875 | 22.704 | −14.786 | 1.00 | 18.20 |
| ATOM | 359 | OD1 | ASN | 46 | 1.395 | 21.610 | −14.470 | 1.00 | 31.96 |
| ATOM | 360 | ND2 | ASN | 46 | 1.269 | 23.750 | −15.306 | 1.00 | 24.56 |
| ATOM | 361 | C | ASN | 46 | 5.043 | 23.111 | −12.634 | 1.00 | 8.17 |
| ATOM | 362 | O | ASN | 46 | 5.936 | 22.860 | −13.449 | 1.00 | 10.28 |
| ATOM | 363 | N | HIS | 47 | 5.323 | 23.425 | −11.366 | 1.00 | 8.42 |
| ATOM | 364 | CA | HIS | 47 | 6.663 | 23.646 | −10.843 | 1.00 | 8.14 |
| ATOM | 365 | CB | HIS | 47 | 6.618 | 24.724 | −9.775 | 1.00 | 8.35 |
| ATOM | 366 | CG | HIS | 47 | 5.590 | 24.430 | −8.727 | 1.00 | 7.34 |
| ATOM | 367 | CD2 | HIS | 47 | 4.399 | 25.000 | −8.504 | 1.00 | 7.88 |
| ATOM | 368 | ND1 | HIS | 47 | 5.719 | 23.383 | −7.812 | 1.00 | 7.39 |
| ATOM | 369 | CE1 | HIS | 47 | 4.626 | 23.360 | −7.052 | 1.00 | 8.04 |
| ATOM | 370 | NE2 | HIS | 47 | 3.827 | 24.344 | −7.440 | 1.00 | 8.14 |
| ATOM | 371 | C | HIS | 47 | 7.375 | 22.430 | −10.325 | 1.00 | 7.89 |
| ATOM | 372 | O | HIS | 47 | 8.580 | 22.464 | −10.091 | 1.00 | 9.85 |
| ATOM | 373 | N | GLY | 48 | 6.691 | 21.328 | −10.139 | 1.00 | 8.33 |
| ATOM | 374 | CA | GLY | 48 | 7.238 | 20.053 | −9.749 | 1.00 | 8.83 |
| ATOM | 375 | C | GLY | 48 | 7.522 | 19.849 | −8.282 | 1.00 | 8.46 |
| ATOM | 376 | O | GLY | 48 | 8.060 | 18.780 | −7.953 | 1.00 | 11.24 |
| ATOM | 377 | N | ILE | 49 | 7.267 | 20.817 | −7.412 | 1.00 | 7.06 |
| ATOM | 378 | CA | ILE | 49 | 7.568 | 20.636 | −6.015 | 1.00 | 6.70 |
| ATOM | 379 | CB | ILE | 49 | 8.093 | 21.962 | −5.389 | 1.00 | 7.04 |
| ATOM | 380 | CG2 | ILE | 49 | 8.286 | 21.829 | −3.886 | 1.00 | 7.67 |
| ATOM | 381 | CG1 | ILE | 49 | 9.383 | 22.383 | −6.077 | 1.00 | 8.13 |
| ATOM | 382 | CD1 | ILE | 49 | 9.964 | 23.699 | −5.584 | 1.00 | 9.61 |
| ATOM | 383 | C | ILE | 49 | 6.327 | 20.166 | −5.245 | 1.00 | 6.55 |
| ATOM | 384 | O | ILE | 49 | 5.210 | 20.605 | −5.497 | 1.00 | 7.34 |
| ATOM | 385 | N | ASN | 50 | 6.531 | 19.246 | −4.311 | 1.00 | 6.14 |
| ATOM | 386 | CA | ASN | 50 | 5.449 | 18.729 | −3.464 | 1.00 | 5.89 |
| ATOM | 387 | CB | ASN | 50 | 5.834 | 17.332 | −2.967 | 1.00 | 6.25 |
| ATOM | 388 | CG | ASN | 50 | 4.688 | 16.658 | −2.270 | 1.00 | 5.80 |
| ATOM | 389 | OD1 | ASN | 50 | 3.717 | 17.297 | −1.870 | 1.00 | 7.60 |
| ATOM | 390 | ND2 | ASN | 50 | 4.840 | 15.366 | −2.058 | 1.00 | 7.26 |
| ATOM | 391 | C | ASN | 50 | 5.184 | 19.714 | −2.331 | 1.00 | 5.68 |
| ATOM | 392 | O | ASN | 50 | 5.744 | 19.597 | −1.230 | 1.00 | 6.62 |
| ATOM | 393 | N | VAL | 51 | 4.328 | 20.676 | −2.619 | 1.00 | 5.97 |
| ATOM | 394 | CA | VAL | 51 | 4.037 | 21.706 | −1.632 | 1.00 | 6.10 |
| ATOM | 395 | CB | VAL | 51 | 3.508 | 23.010 | −2.290 | 1.00 | 6.74 |
| ATOM | 396 | CG1 | VAL | 51 | 4.557 | 23.676 | −3.184 | 1.00 | 9.06 |
| ATOM | 397 | CG2 | VAL | 51 | 2.250 | 22.743 | −3.048 | 1.00 | 9.15 |
| ATOM | 398 | C | VAL | 51 | 3.137 | 21.232 | −0.504 | 1.00 | 7.22 |
| ATOM | 399 | O | VAL | 51 | 3.199 | 21.758 | 0.610 | 1.00 | 7.84 |
| ATOM | 400 | N | GLN | 52 | 2.286 | 20.231 | −0.761 | 1.00 | 7.44 |
| ATOM | 401 | CA | GLN | 52 | 1.474 | 19.721 | 0.339 | 1.00 | 6.88 |
| ATOM | 402 | CB | GLN | 52 | 0.442 | 18.728 | −0.163 | 1.00 | 8.70 |
| ATOM | 403 | CG | GLN | 52 | −0.534 | 18.205 | 0.917 | 1.00 | 10.71 |
| ATOM | 404 | CD | GLN | 52 | −0.066 | 17.059 | 1.807 | 1.00 | 12.39 |
| ATOM | 405 | OE1 | GLN | 52 | 0.970 | 16.387 | 1.579 | 1.00 | 12.25 |
| ATOM | 406 | NE2 | GLN | 52 | −0.672 | 16.925 | 2.956 | 1.00 | 13.45 |
| ATOM | 407 | C | GLN | 52 | 2.410 | 19.094 | 1.378 | 1.00 | 6.81 |
| ATOM | 408 | O | GLN | 52 | 2.162 | 19.264 | 2.599 | 1.00 | 6.97 |
| ATOM | 409 | N | ARG | 53 | 3.434 | 18.364 | 0.950 | 1.00 | 6.70 |
| ATOM | 410 | CA | ARG | 53 | 4.339 | 17.734 | 1.912 | 1.00 | 6.00 |
| ATOM | 411 | CB | ARG | 53 | 5.152 | 16.655 | 1.210 | 1.00 | 6.55 |
| ATOM | 412 | CG | ARG | 53 | 6.068 | 15.894 | 2.129 | 1.00 | 6.59 |
| ATOM | 413 | CD | ARG | 53 | 6.645 | 14.676 | 1.432 | 1.00 | 8.72 |
| ATOM | 414 | NE | ARG | 53 | 7.445 | 13.846 | 2.348 | 1.00 | 8.70 |
| ATOM | 415 | CZ | ARG | 53 | 8.771 | 13.910 | 2.425 | 1.00 | 10.05 |
| ATOM | 416 | NH1 | ARG | 53 | 9.464 | 14.723 | 1.605 | 1.00 | 12.80 |
| ATOM | 417 | NH2 | ARG | 53 | 9.424 | 13.106 | 3.279 | 1.00 | 10.00 |
| ATOM | 418 | C | ARG | 53 | 5.156 | 18.809 | 2.598 | 1.00 | 5.84 |
| ATOM | 419 | O | ARG | 53 | 5.396 | 18.698 | 3.820 | 1.00 | 6.89 |
| ATOM | 420 | N | LEU | 54 | 5.598 | 19.850 | 1.903 | 1.00 | 5.98 |
| ATOM | 421 | CA | LEU | 54 | 6.274 | 20.982 | 2.544 | 1.00 | 5.87 |
| ATOM | 422 | CB | LEU | 54 | 6.558 | 22.056 | 1.489 | 1.00 | 6.27 |
| ATOM | 423 | CG | LEU | 54 | 6.940 | 23.435 | 2.017 | 1.00 | 7.00 |
| ATOM | 424 | CD1 | LEU | 54 | 8.286 | 23.396 | 2.689 | 1.00 | 7.98 |
| ATOM | 425 | CD2 | LEU | 54 | 6.864 | 24.441 | 0.866 | 1.00 | 8.66 |
| ATOM | 426 | C | LEU | 54 | 5.406 | 21.576 | 3.660 | 1.00 | 5.97 |
| ATOM | 427 | O | LEU | 54 | 5.870 | 21.822 | 4.788 | 1.00 | 6.89 |
| ATOM | 428 | N | SER | 55 | 4.136 | 21.786 | 3.344 | 1.00 | 6.27 |
| ATOM | 429 | CA | SER | 55 | 3.240 | 22.335 | 4.357 | 1.00 | 6.86 |
| ATOM | 430 | CB | SER | 55 | 1.916 | 22.675 | 3.694 | 1.00 | 7.85 |
| ATOM | 431 | OG | SER | 55 | 0.981 | 23.194 | 4.615 | 1.00 | 11.52 |
| ATOM | 432 | C | SER | 55 | 3.098 | 21.410 | 5.563 | 1.00 | 6.98 |
| ATOM | 433 | O | SER | 55 | 3.081 | 21.834 | 6.713 | 1.00 | 7.09 |
| ATOM | 434 | N | GLN | 56 | 2.961 | 20.107 | 5.309 | 1.00 | 7.22 |
| ATOM | 435 | CA | GLN | 56 | 2.784 | 19.139 | 6.379 | 1.00 | 7.20 |
| ATOM | 436 | CB | GLN | 56 | 2.400 | 17.785 | 5.799 | 1.00 | 9.23 |
| ATOM | 437 | CG | GLN | 56 | 2.329 | 16.657 | 6.795 | 1.00 | 11.52 |
| ATOM | 438 | CD | GLN | 56 | 1.214 | 16.804 | 7.803 | 1.00 | 14.79 |
| ATOM | 439 | OE1 | GLN | 56 | 1.405 | 16.302 | 3.912 | 1.00 | 22.67 |
| ATOM | 440 | NE2 | GLN | 56 | 0.122 | 17.443 | 7.423 | 1.00 | 13.58 |
| ATOM | 441 | C | GLN | 56 | 4.007 | 19.101 | 7.279 | 1.00 | 7.77 |
| ATOM | 442 | O | GLN | 56 | 3.869 | 19.099 | 8.517 | 1.00 | 7.98 |
| ATOM | 443 | N | LYS | 57 | 5.188 | 18.978 | 6.684 | 1.00 | 6.97 |
| ATOM | 444 | CA | LYS | 57 | 6.404 | 18.904 | 7.465 | 1.00 | 7.11 |
| ATOM | 445 | CB | LYS | 57 | 7.622 | 18.583 | 6.598 | 1.00 | 7.89 |
| ATOM | 446 | CG | LYS | 57 | 7.574 | 17.249 | 5.861 | 1.00 | 7.92 |
| ATOM | 447 | CD | LYS | 57 | 7.561 | 16.023 | 6.784 | 1.00 | 9.42 |
| ATOM | 448 | CE | LYS | 57 | 7.650 | 14.765 | 5.962 | 1.00 | 10.57 |
| ATOM | 449 | NZ | LYS | 57 | 7.444 | 13.524 | 6.770 | 1.00 | 13.31 |
| ATOM | 450 | C | LYS | 57 | 6.623 | 20.175 | 8.273 | 1.00 | 6.73 |
| ATOM | 451 | O | LYS | 57 | 7.102 | 20.149 | 9.413 | 1.00 | 8.29 |
| ATOM | 452 | N | THR | 58 | 6.325 | 21.322 | 7.676 | 1.00 | 7.11 |
| ATOM | 453 | CA | THR | 58 | 6.448 | 22.618 | 8.342 | 1.00 | 7.21 |
| ATOM | 454 | CB | THR | 58 | 6.257 | 23.767 | 7.355 | 1.00 | 7.45 |
| ATOM | 455 | OG1 | THR | 58 | 7.318 | 23.725 | 6.392 | 1.00 | 8.22 |
| ATOM | 456 | CG2 | THR | 58 | 6.316 | 25.134 | 8.054 | 1.00 | 8.05 |
| ATOM | 457 | C | THR | 58 | 5.495 | 22.727 | 9.527 | 1.00 | 6.99 |
| ATOM | 458 | O | THR | 58 | 5.879 | 23.194 | 10.618 | 1.00 | 7.76 |
| ATOM | 459 | N | LYS | 59 | 4.257 | 22.255 | 9.336 | 1.00 | 7.98 |
| ATOM | 460 | CA | LYS | 59 | 3.270 | 22.244 | 10.430 | 1.00 | 8.41 |
| ATOM | 461 | CB | LYS | 59 | 1.933 | 21.732 | 9.870 | 1.00 | 11.31 |
| ATOM | 462 | CG | LYS | 59 | 0.857 | 21.678 | 10.962 | 1.00 | 17.36 |
| ATOM | 463 | CD | LYS | 59 | −0.412 | 21.032 | 10.378 | 1.00 | 21.57 |
| ATOM | 464 | CE | LYS | 59 | −0.145 | 19.572 | 10.080 | 1.00 | 26.63 |
| ATOM | 465 | NZ | LYS | 59 | 0.949 | 19.018 | 10.953 | 1.00 | 41.28 |
| ATOM | 466 | C | LYS | 59 | 3.756 | 21.375 | 11.583 | 1.00 | 8.97 |
| ATOM | 467 | O | LYS | 59 | 3.662 | 21.789 | 12.743 | 1.00 | 9.25 |
| ATOM | 468 | N | GLU | 60 | 4.260 | 20.182 | 11.254 | 1.00 | 8.73 |
| ATOM | 469 | CA | GLU | 60 | 4.763 | 19.297 | 12.288 | 1.00 | 9.20 |
| ATOM | 470 | CB | GLU | 60 | 5.286 | 17.988 | 11.679 | 1.00 | 10.60 |
| ATOM | 471 | CG | GLU | 60 | 4.189 | 17.114 | 11.083 | 1.00 | 13.69 |
| ATOM | 472 | CD | GLU | 60 | 4.634 | 15.954 | 10.241 | 1.00 | 15.34 |
| ATOM | 473 | OE1 | GLU | 60 | 5.846 | 15.700 | 10.211 | 1.00 | 20.83 |
| ATOM | 474 | OE2 | GLU | 60 | 3.819 | 15.290 | 9.550 | 1.00 | 19.41 |
| ATOM | 475 | C | GLU | 60 | 5.849 | 19.961 | 13.119 | 1.00 | 10.04 |
| ATOM | 476 | O | GLU | 60 | 5.822 | 19.898 | 14.353 | 1.00 | 11.42 |
| ATOM | 477 | N | PHE | 61 | 6.751 | 20.687 | 12.465 | 1.00 | 8.91 |
| ATOM | 478 | CA | PHE | 61 | 7.785 | 21.443 | 13.184 | 1.00 | 7.35 |
| ATOM | 479 | CB | PHE | 61 | 8.775 | 21.958 | 12.129 | 1.00 | 7.73 |
| ATOM | 480 | CG | PHE | 61 | 9.763 | 22.977 | 12.665 | 1.00 | 8.40 |
| ATOM | 481 | CD1 | PHE | 61 | 10.749 | 22.594 | 13.521 | 1.00 | 9.42 |
| ATOM | 482 | CD2 | PHE | 61 | 9.667 | 24.316 | 12.302 | 1.00 | 10.73 |
| ATOM | 483 | CE1 | PHE | 61 | 11.653 | 23.520 | 13.994 | 1.00 | 10.45 |
| ATOM | 484 | CE2 | PHE | 61 | 10.591 | 25.256 | 12.727 | 1.00 | 11.37 |
| ATOM | 485 | CZ | PHE | 61 | 11.562 | 24.834 | 13.606 | 1.00 | 10.57 |
| ATOM | 486 | C | PHE | 61 | 7.211 | 22.579 | 14.015 | 1.00 | 7.60 |
| ATOM | 487 | O | PHE | 61 | 7.474 | 22.658 | 15.228 | 1.00 | 8.53 |
| ATOM | 488 | N | HIS | 62 | 6.446 | 23.487 | 13.426 | 1.00 | 8.25 |
| ATOM | 489 | CA | HIS | 62 | 5.921 | 24.635 | 14.156 | 1.00 | 8.66 |
| ATOM | 490 | CB | HIS | 62 | 5.076 | 25.531 | 13.261 | 1.00 | 9.03 |
| ATOM | 491 | CG | HIS | 62 | 5.800 | 26.423 | 12.311 | 1.00 | 8.36 |
| ATOM | 492 | CD2 | HIS | 62 | 5.271 | 26.774 | 11.094 | 1.00 | 7.90 |
| ATOM | 493 | ND1 | HIS | 62 | 6.963 | 27.124 | 12.415 | 1.00 | 10.22 |
| ATOM | 494 | CE1 | HIS | 62 | 7.123 | 27.845 | 11.303 | 1.00 | 7.76 |
| ATOM | 495 | NE2 | HIS | 62 | 6.122 | 27.622 | 10.488 | 1.00 | 10.59 |
| ATOM | 496 | C | HIS | 62 | 5.091 | 24.264 | 15.385 | 1.00 | 9.71 |
| ATOM | 497 | O | HIS | 62 | 5.070 | 24.974 | 16.376 | 1.00 | 11.19 |
| ATOM | 498 | N | MET | 63 | 4.335 | 23.167 | 15.283 | 1.00 | 10.41 |
| ATOM | 499 | CA | MET | 63 | 3.393 | 22.836 | 16.321 | 1.00 | 12.27 |
| ATOM | 500 | CB | MET | 63 | 2.151 | 22.162 | 15.705 | 1.00 | 13.32 |
| ATOM | 501 | CG | MET | 63 | 1.453 | 23.061 | 14.692 | 1.00 | 14.13 |
| ATOM | 502 | SD | MET | 63 | 1.062 | 24.757 | 15.253 | 1.00 | 21.44 |
| ATOM | 503 | CE | MET | 63 | 0.528 | 25.519 | 13.715 | 1.00 | 41.15 |
| ATOM | 504 | C | MET | 63 | 4.020 | 22.008 | 17.416 | 1.00 | 13.21 |
| ATOM | 505 | O | MET | 63 | 3.383 | 21.818 | 18.470 | 1.00 | 19.62 |
| ATOM | 506 | N | SER | 64 | 5.203 | 21.457 | 17.178 | 1.00 | 10.61 |
| ATOM | 507 | CA | SER | 64 | 5.896 | 20.662 | 18.191 | 1.00 | 13.44 |
| ATOM | 508 | CB | SER | 64 | 6.289 | 19.283 | 17.662 | 1.00 | 15.70 |
| ATOM | 509 | OG | SER | 64 | 7.299 | 19.356 | 16.689 | 1.00 | 17.37 |
| ATOM | 510 | C | SER | 64 | 7.105 | 21.342 | 18.809 | 1.00 | 12.71 |
| ATOM | 511 | O | SER | 64 | 7.528 | 20.933 | 19.888 | 1.00 | 16.31 |
| ATOM | 512 | N | ILE | 65 | 7.664 | 22.392 | 18.236 | 1.00 | 10.13 |
| ATOM | 513 | CA | ILE | 65 | 8.799 | 23.010 | 18.900 | 1.00 | 9.79 |
| ATOM | 514 | CB | ILE | 65 | 9.469 | 23.968 | 17.906 | 1.00 | 10.92 |
| ATOM | 515 | CG2 | ILE | 65 | 8.583 | 25.072 | 17.406 | 1.00 | 13.10 |
| ATOM | 516 | CG1 | ILE | 65 | 10.787 | 24.455 | 18.502 | 1.00 | 11.85 |
| ATOM | 517 | CD1 | ILE | 65 | 11.740 | 25.156 | 17.598 | 1.00 | 13.44 |
| ATOM | 518 | C | ILE | 65 | 8.366 | 23.703 | 20.162 | 1.00 | 10.18 |
| ATOM | 519 | O | ILE | 65 | 7.286 | 24.267 | 20.263 | 1.00 | 12.62 |
| ATOM | 520 | N | THR | 66 | 9.170 | 23.608 | 21.194 | 1.00 | 10.22 |
| ATOM | 521 | CA | THR | 66 | 8.866 | 24.106 | 22.535 | 1.00 | 10.08 |
| ATOM | 522 | CB | THR | 66 | 9.278 | 23.065 | 23.583 | 1.00 | 9.97 |
| ATOM | 523 | OG1 | THR | 66 | 10.681 | 22.892 | 23.511 | 1.00 | 12.07 |
| ATOM | 524 | CG2 | THR | 66 | 8.571 | 21.749 | 23.373 | 1.00 | 14.54 |
| ATOM | 525 | C | THR | 66 | 9.559 | 25.420 | 22.846 | 1.00 | 8.74 |
| ATOM | 526 | O | THR | 66 | 10.531 | 25.775 | 22.160 | 1.00 | 8.66 |
| ATOM | 527 | N | PRO | 67 | 9.078 | 26.142 | 23.864 | 1.00 | 9.12 |
| ATOM | 528 | CD | PRO | 67 | 7.813 | 25.938 | 24.594 | 1.00 | 11.31 |
| ATOM | 529 | CA | PRO | 67 | 9.724 | 27.392 | 24.251 | 1.00 | 9.65 |
| ATOM | 530 | CB | PRO | 67 | 8.925 | 27.860 | 25.450 | 1.00 | 12.19 |
| ATOM | 531 | CG | PRO | 67 | 7.598 | 27.233 | 25.312 | 1.00 | 12.99 |
| ATOM | 532 | C | PRO | 67 | 11.209 | 27.228 | 24.567 | 1.00 | 9.34 |
| ATOM | 533 | O | PRO | 67 | 12.046 | 28.064 | 24.200 | 1.00 | 10.59 |
| ATOM | 534 | N | GLU | 68 | 11.597 | 26.115 | 25.186 | 1.00 | 10.47 |
| ATOM | 535 | CA | GLU | 68 | 13.000 | 25.861 | 25.470 | 1.00 | 11.64 |
| ATOM | 536 | CB | GLU | 68 | 13.061 | 24.533 | 26.244 | 1.00 | 13.13 |
| ATOM | 537 | CG | GLU | 68 | 14.452 | 24.123 | 26.600 | 1.00 | 14.22 |
| ATOM | 538 | CD | GLU | 68 | 14.498 | 22.689 | 27.107 | 1.00 | 15.39 |
| ATOM | 539 | OE1 | GLU | 68 | 13.945 | 21.743 | 26.508 | 1.00 | 21.01 |
| ATOM | 540 | OE2 | GLU | 68 | 15.043 | 22.449 | 28.180 | 1.00 | 19.65 |
| ATOM | 541 | C | GLU | 68 | 13.836 | 25.799 | 24.217 | 1.00 | 9.32 |
| ATOM | 542 | O | GLU | 68 | 14.920 | 26.376 | 24.096 | 1.00 | 11.18 |
| ATOM | 543 | N | GLU | 69 | 13.319 | 25.065 | 23.221 | 1.00 | 8.89 |
| ATOM | 544 | CA | GLU | 69 | 14.015 | 24.920 | 21.929 | 1.00 | 9.17 |
| ATOM | 545 | CB | GLU | 69 | 13.310 | 23.921 | 21.010 | 1.00 | 9.44 |
| ATOM | 546 | CG | GLU | 69 | 13.338 | 22.513 | 21.576 | 1.00 | 10.83 |
| ATOM | 547 | CD | GLU | 69 | 12.432 | 21.627 | 20.745 | 1.00 | 12.92 |
| ATOM | 548 | OE1 | GLU | 69 | 12.996 | 20.941 | 19.876 | 1.00 | 19.22 |
| ATOM | 549 | OE2 | GLU | 69 | 11.213 | 21.599 | 21.003 | 1.00 | 19.62 |
| ATOM | 550 | C | GLU | 69 | 14.157 | 26.247 | 21.213 | 1.00 | 8.87 |
| ATOM | 551 | O | GLU | 69 | 15.153 | 26.521 | 20.573 | 1.00 | 9.17 |
| ATOM | 552 | N | LYS | 70 | 13.126 | 27.086 | 21.311 | 1.00 | 8.65 |
| ATOM | 553 | CA | LYS | 70 | 13.148 | 28.387 | 20.621 | 1.00 | 8.41 |
| ATOM | 554 | CB | LYS | 70 | 11.786 | 29.100 | 20.695 | 1.00 | 8.73 |
| ATOM | 555 | CG | LYS | 70 | 10.663 | 28.358 | 19.977 | 1.00 | 8.40 |
| ATOM | 556 | CD | LYS | 70 | 9.319 | 29.001 | 20.239 | 1.00 | 9.16 |
| ATOM | 557 | CE | LYS | 70 | 8.198 | 28.102 | 19.726 | 1.00 | 10.86 |
| ATOM | 558 | NZ | LYS | 70 | 6.875 | 28.741 | 19.900 | 1.00 | 12.00 |
| ATOM | 559 | C | LYS | 70 | 14.268 | 29.257 | 21.182 | 1.00 | 7.77 |
| ATOM | 560 | O | LYS | 70 | 14.992 | 29.885 | 20.400 | 1.00 | 8.10 |
| ATOM | 561 | N | TRP | 71 | 14.418 | 29.325 | 22.514 | 1.00 | 7.55 |
| ATOM | 562 | CA | TRP | 71 | 15.556 | 30.080 | 23.057 | 1.00 | 7.86 |
| ATOM | 563 | CB | TRP | 71 | 15.545 | 30.104 | 24.582 | 1.00 | 8.53 |
| ATOM | 564 | CG | TRP | 71 | 14.467 | 30.932 | 25.195 | 1.00 | 7.80 |
| ATOM | 565 | CD2 | TRP | 71 | 14.216 | 32.335 | 25.045 | 1.00 | 7.55 |
| ATOM | 566 | CE2 | TRP | 71 | 13.097 | 32.672 | 25.824 | 1.00 | 8.56 |
| ATOM | 567 | CE3 | TRP | 71 | 14.813 | 33.362 | 24.326 | 1.00 | 8.26 |
| ATOM | 568 | CD1 | TRP | 71 | 13.512 | 30.477 | 26.068 | 1.00 | 8.54 |
| ATOM | 569 | NE1 | TRP | 71 | 12.682 | 31.521 | 26.446 | 1.00 | 9.24 |
| ATOM | 570 | CZ2 | TRP | 71 | 12.589 | 33.965 | 25.900 | 1.00 | 9.23 |
| ATOM | 571 | CZ3 | TRP | 71 | 14.311 | 34.644 | 24.384 | 1.00 | 8.95 |
| ATOM | 572 | CH2 | TRP | 71 | 13.198 | 34.947 | 25.181 | 1.00 | 9.37 |
| ATOM | 573 | C | TRP | 71 | 16.892 | 29.513 | 22.565 | 1.00 | 7.41 |
| ATOM | 574 | O | TRP | 71 | 17.808 | 30.271 | 22.223 | 1.00 | 7.75 |
| ATOM | 575 | N | ASP | 72 | 16.954 | 28.180 | 22.552 | 1.00 | 7.95 |
| ATOM | 576 | CA | ASP | 72 | 18.195 | 27.508 | 22.215 | 1.00 | 9.24 |
| ATOM | 577 | CB | ASP | 72 | 18.123 | 26.031 | 22.638 | 1.00 | 11.24 |
| ATOM | 578 | CG | ASP | 72 | 18.208 | 25.822 | 24.146 | 1.00 | 14.08 |
| ATOM | 579 | OD1 | ASP | 72 | 18.351 | 26.778 | 24.967 | 1.00 | 15.23 |
| ATOM | 580 | OD2 | ASP | 72 | 17.942 | 24.678 | 24.599 | 1.00 | 17.93 |
| ATOM | 581 | C | ASP | 72 | 18.589 | 27.715 | 20.764 | 1.00 | 9.14 |
| ATOM | 582 | O | ASP | 72 | 19.766 | 27.648 | 20.413 | 1.00 | 10.16 |
| ATOM | 583 | N | LEU | 73 | 17.615 | 27.980 | 19.872 | 1.00 | 7.62 |
| ATOM | 584 | CA | LEU | 73 | 17.828 | 28.233 | 18.474 | 1.00 | 8.24 |
| ATOM | 585 | CB | LEU | 73 | 16.767 | 27.452 | 17.648 | 1.00 | 9.44 |
| ATOM | 586 | CG | LEU | 73 | 16.895 | 25.941 | 17.683 | 1.00 | 10.93 |
| ATOM | 587 | CD1 | LEU | 73 | 15.676 | 25.238 | 17.093 | 1.00 | 16.41 |
| ATOM | 588 | CD2 | LEU | 73 | 18.199 | 25.437 | 17.077 | 1.00 | 14.97 |
| ATOM | 589 | C | LEU | 73 | 17.804 | 29.708 | 18.102 | 1.00 | 7.12 |
| ATOM | 590 | O | LEU | 73 | 17.930 | 30.074 | 16.935 | 1.00 | 7.82 |
| ATOM | 591 | N | ALA | 74 | 17.541 | 30.607 | 19.036 | 1.00 | 6.72 |
| ATOM | 592 | CA | ALA | 74 | 17.201 | 31.967 | 18.766 | 1.00 | 6.46 |
| ATOM | 593 | CB | ALA | 74 | 16.742 | 32.662 | 20.042 | 1.00 | 7.96 |
| ATOM | 594 | C | ALA | 74 | 18.258 | 32.818 | 18.098 | 1.00 | 7.50 |
| ATOM | 595 | O | ALA | 74 | 19.423 | 32.697 | 18.490 | 1.00 | 8.38 |
| ATOM | 596 | N | ILE | 75 | 17.864 | 33.689 | 17.172 | 1.00 | 6.76 |
| ATOM | 597 | CA | ILE | 75 | 18.795 | 34.680 | 16.652 | 1.00 | 6.71 |
| ATOM | 598 | CB | ILE | 75 | 18.224 | 35.376 | 15.420 | 1.00 | 6.88 |
| ATOM | 599 | CG2 | ILE | 75 | 18.044 | 34.424 | 14.239 | 1.00 | 8.21 |
| ATOM | 600 | CG1 | ILE | 75 | 16.936 | 36.140 | 15.755 | 1.00 | 7.17 |
| ATOM | 601 | CD1 | ILE | 75 | 16.522 | 37.151 | 14.715 | 1.00 | 9.71 |
| ATOM | 602 | C | ILE | 75 | 19.201 | 35.704 | 17.734 | 1.00 | 7.09 |
| ATOM | 603 | O | ILE | 75 | 18.546 | 35.810 | 18.781 | 1.00 | 6.89 |
| ATOM | 604 | N | ALA | 76 | 20.280 | 36.421 | 17.461 | 1.00 | 8.25 |
| ATOM | 605 | CA | ALA | 76 | 20.876 | 37.407 | 18.360 | 1.00 | 9.65 |
| ATOM | 606 | CB | ALA | 76 | 22.084 | 38.067 | 17.666 | 1.00 | 16.12 |
| ATOM | 607 | C | ALA | 76 | 19.886 | 38.445 | 18.838 | 1.00 | 8.48 |
| ATOM | 608 | O | ALA | 76 | 19.962 | 38.940 | 19.953 | 1.00 | 9.39 |
| ATOM | 609 | N | ALA | 77 | 18.905 | 38.810 | 18.002 | 1.00 | 8.30 |
| ATOM | 610 | CA | ALA | 77 | 17.911 | 39.800 | 18.374 | 1.00 | 9.45 |
| ATOM | 611 | CB | ALA | 77 | 16.992 | 40.064 | 17.179 | 1.00 | 10.84 |
| ATOM | 612 | C | ALA | 77 | 17.100 | 39.352 | 19.582 | 1.00 | 8.44 |
| ATOM | 613 | O | ALA | 77 | 16.541 | 40.191 | 20.299 | 1.00 | 10.71 |
| ATOM | 614 | N | TYR | 78 | 16.967 | 38.049 | 19.816 | 1.00 | 7.56 |
| ATOM | 615 | CA | TYR | 78 | 16.222 | 37.513 | 20.938 | 1.00 | 8.24 |
| ATOM | 616 | CB | TYR | 78 | 15.223 | 36.420 | 20.451 | 1.00 | 7.69 |
| ATOM | 617 | CG | TYR | 78 | 14.158 | 37.043 | 19.575 | 1.00 | 7.53 |
| ATOM | 618 | CD1 | TYR | 78 | 14.272 | 36.972 | 18.193 | 1.00 | 7.61 |
| ATOM | 619 | CE1 | TYR | 78 | 13.370 | 37.508 | 17.335 | 1.00 | 7.49 |
| ATOM | 620 | CD2 | TYR | 78 | 13.050 | 37.728 | 20.079 | 1.00 | 8.66 |
| ATOM | 621 | CE2 | TYR | 78 | 12.150 | 38.290 | 19.216 | 1.00 | 9.02 |
| ATOM | 622 | CZ | TYR | 78 | 12.291 | 38.173 | 17.845 | 1.00 | 8.23 |
| ATOM | 623 | OH | TYR | 78 | 11.357 | 38.805 | 17.028 | 1.00 | 10.37 |
| ATOM | 624 | C | TYR | 78 | 17.115 | 36.920 | 22.016 | 1.00 | 7.16 |
| ATOM | 625 | O | TYR | 78 | 16.681 | 36.716 | 23.134 | 1.00 | 10.37 |
| ATOM | 626 | N | ASN | 79 | 18.346 | 36.542 | 21.694 | 1.00 | 8.52 |
| ATOM | 627 | CA | ASN | 79 | 19.232 | 35.854 | 22.624 | 1.00 | 7.53 |
| ATOM | 628 | CB | ASN | 79 | 19.164 | 34.338 | 22.379 | 1.00 | 7.59 |
| ATOM | 629 | CG | ASN | 79 | 20.000 | 33.518 | 23.343 | 1.00 | 7.82 |
| ATOM | 630 | OD1 | ASN | 79 | 20.942 | 34.010 | 23.962 | 1.00 | 8.62 |
| ATOM | 631 | ND2 | ASN | 79 | 19.686 | 32.222 | 23.477 | 1.00 | 9.18 |
| ATOM | 632 | C | ASN | 79 | 20.653 | 36.401 | 22.394 | 1.00 | 8.94 |
| ATOM | 633 | O | ASN | 79 | 21.341 | 36.042 | 21.442 | 1.00 | 10.00 |
| ATOM | 634 | N | LYS | 80 | 21.086 | 37.226 | 23.348 | 1.00 | 9.67 |
| ATOM | 635 | CA | LYS | 80 | 22.403 | 37.853 | 23.279 | 1.00 | 11.21 |
| ATOM | 636 | CB | LYS | 80 | 22.575 | 38.808 | 24.473 | 1.00 | 14.70 |
| ATOM | 637 | CG | LYS | 80 | 21.697 | 40.035 | 24.385 | 1.00 | 20.93 |
| ATOM | 638 | CD | LYS | 80 | 21.970 | 41.007 | 25.532 | 1.00 | 25.64 |
| ATOM | 639 | CE | LYS | 80 | 21.540 | 42.420 | 25.219 | 1.00 | 29.22 |
| ATOM | 640 | NZ | LYS | 80 | 20.209 | 42.711 | 25.795 | 1.00 | 39.86 |
| ATOM | 641 | C | LYS | 80 | 23.565 | 36.877 | 23.274 | 1.00 | 10.80 |
| ATOM | 642 | O | LYS | 80 | 24.702 | 37.192 | 22.944 | 1.00 | 13.45 |
| ATOM | 643 | N | GLU | 81 | 23.349 | 35.630 | 23.706 | 1.00 | 10.06 |
| ATOM | 644 | CA | GLU | 81 | 24.436 | 34.666 | 23.722 | 1.00 | 11.25 |
| ATOM | 645 | CB | GLU | 81 | 24.060 | 33.385 | 24.505 | 1.00 | 11.80 |
| ATOM | 646 | CG | GLU | 81 | 23.668 | 33.565 | 25.948 | 1.00 | 15.18 |
| ATOM | 647 | CD | GLU | 81 | 23.394 | 32.322 | 26.768 | 1.00 | 16.40 |
| ATOM | 648 | OE1 | GLU | 81 | 22.810 | 32.379 | 27.889 | 1.00 | 16.96 |
| ATOM | 649 | OE2 | GLU | 81 | 23.688 | 31.185 | 26.315 | 1.00 | 21.03 |
| ATOM | 650 | C | GLU | 81 | 24.791 | 34.265 | 22.281 | 1.00 | 12.20 |
| ATOM | 651 | O | GLU | 81 | 25.838 | 33.655 | 22.069 | 1.00 | 14.12 |
| ATOM | 652 | N | HIS | 82 | 23.900 | 34.439 | 21.316 | 1.00 | 10.21 |
| ATOM | 653 | CA | HIS | 82 | 24.112 | 33.865 | 19.980 | 1.00 | 9.63 |
| ATOM | 654 | CB | HIS | 82 | 22.803 | 33.231 | 19.530 | 1.00 | 9.27 |
| ATOM | 655 | CG | HIS | 82 | 22.371 | 32.070 | 20.360 | 1.00 | 9.26 |
| ATOM | 656 | CD2 | HIS | 82 | 23.068 | 31.323 | 21.257 | 1.00 | 10.67 |
| ATOM | 657 | ND1 | HIS | 82 | 21.113 | 31.516 | 20.288 | 1.00 | 8.55 |
| ATOM | 658 | CE1 | HIS | 82 | 21.063 | 30.500 | 21.141 | 1.00 | 8.43 |
| ATOM | 659 | NE2 | HIS | 82 | 22.223 | 30.366 | 21.742 | 1.00 | 10.55 |
| ATOM | 660 | C | HIS | 82 | 24.567 | 34.933 | 19.029 | 1.00 | 10.23 |
| ATOM | 661 | O | HIS | 82 | 23.901 | 35.266 | 18.055 | 1.00 | 11.36 |
| ATOM | 662 | N | GLN | 83 | 25.726 | 35.507 | 19.295 | 1.00 | 12.56 |
| ATOM | 663 | CA | GLN | 83 | 26.203 | 36.634 | 18.507 | 1.00 | 13.52 |
| ATOM | 664 | CB | GLN | 83 | 27.470 | 37.164 | 19.181 | 1.00 | 16.22 |
| ATOM | 665 | CG | GLN | 83 | 27.163 | 37.925 | 20.478 | 1.00 | 19.23 |
| ATOM | 666 | CD | GLN | 83 | 26.294 | 39.148 | 20.300 | 1.00 | 20.38 |
| ATOM | 667 | OE1 | GLN | 83 | 26.558 | 40.074 | 19.536 | 1.00 | 29.81 |
| ATOM | 668 | NE2 | GLN | 83 | 25.191 | 39.208 | 21.046 | 1.00 | 22.46 |
| ATOM | 669 | C | GLN | 83 | 26.450 | 36.332 | 17.050 | 1.00 | 13.44 |
| ATOM | 670 | O | GLN | 83 | 26.392 | 37.262 | 16.246 | 1.00 | 15.10 |
| ATOM | 671 | N | ASP | 84 | 26.636 | 35.089 | 16.694 | 1.00 | 13.17 |
| ATOM | 672 | CA | ASP | 84 | 26.862 | 34.722 | 15.292 | 1.00 | 14.01 |
| ATOM | 673 | CB | ASP | 84 | 27.692 | 33.451 | 15.162 | 1.00 | 17.37 |
| ATOM | 674 | CG | ASP | 84 | 29.128 | 33.607 | 15.603 | 1.00 | 20.70 |
| ATOM | 675 | OD1 | ASP | 84 | 29.618 | 34.762 | 15.567 | 1.00 | 24.40 |
| ATOM | 676 | OD2 | ASP | 84 | 29.754 | 32.593 | 15.997 | 1.00 | 26.32 |
| ATOM | 677 | C | ASP | 84 | 25.612 | 34.566 | 14.450 | 1.00 | 13.94 |
| ATOM | 678 | O | ASP | 84 | 25.668 | 34.506 | 13.229 | 1.00 | 13.72 |
| ATOM | 679 | N | GLN | 85 | 24.472 | 34.468 | 15.125 | 1.00 | 12.76 |
| ATOM | 680 | CA | GLN | 85 | 23.186 | 34.270 | 14.473 | 1.00 | 11.48 |
| ATOM | 681 | CB | GLN | 85 | 22.324 | 33.390 | 15.381 | 1.00 | 11.21 |
| ATOM | 682 | CG | GLN | 85 | 22.791 | 31.971 | 15.552 | 1.00 | 11.91 |
| ATOM | 683 | CD | GLN | 85 | 21.795 | 31.109 | 16.278 | 1.00 | 10.83 |
| ATOM | 684 | OE1 | GLN | 85 | 20.636 | 30.970 | 15.841 | 1.00 | 12.23 |
| ATOM | 685 | NE2 | GLN | 85 | 22.225 | 30.557 | 17.380 | 1.00 | 9.17 |
| ATOM | 686 | C | GLN | 85 | 22.468 | 35.584 | 14.216 | 1.00 | 12.01 |
| ATOM | 687 | O | GLN | 85 | 21.590 | 36.041 | 14.977 | 1.00 | 14.96 |
| ATOM | 688 | N | VAL | 86 | 22.773 | 36.203 | 13.091 | 1.00 | 10.72 |
| ATOM | 689 | CA | VAL | 86 | 22.062 | 37.392 | 12.684 | 1.00 | 11.56 |
| ATOM | 690 | CB | VAL | 86 | 23.031 | 38.325 | 11.951 | 1.00 | 12.29 |
| ATOM | 691 | CG1 | VAL | 86 | 22.341 | 39.541 | 11.368 | 1.00 | 16.28 |
| ATOM | 692 | CG2 | VAL | 86 | 24.227 | 38.725 | 12.807 | 1.00 | 17.21 |
| ATOM | 693 | C | VAL | 86 | 20.862 | 36.994 | 11.853 | 1.00 | 11.74 |
| ATOM | 694 | O | VAL | 86 | 19.737 | 37.435 | 12.126 | 1.00 | 17.43 |
| ATOM | 695 | N | ARG | 87 | 21.060 | 36.084 | 10.898 | 1.00 | 8.59 |
| ATOM | 696 | CA | ARG | 87 | 20.044 | 35.624 | 9.972 | 1.00 | 7.72 |
| ATOM | 697 | CB | ARG | 87 | 20.635 | 35.441 | 8.553 | 1.00 | 8.11 |
| ATOM | 698 | CG | ARG | 87 | 21.203 | 36.706 | 7.959 | 1.00 | 8.47 |
| ATOM | 699 | CD | ARG | 87 | 21.842 | 36.462 | 6.599 | 1.00 | 7.75 |
| ATOM | 700 | NE | ARG | 87 | 20.829 | 36.173 | 5.609 | 1.00 | 7.39 |
| ATOM | 701 | CZ | ARG | 87 | 21.042 | 35.691 | 4.396 | 1.00 | 6.65 |
| ATOM | 702 | NH1 | ARG | 87 | 22.260 | 35.389 | 3.959 | 1.00 | 8.68 |
| ATOM | 703 | NH2 | ARG | 87 | 20.036 | 35.471 | 3.547 | 1.00 | 7.51 |
| ATOM | 704 | C | ARG | 87 | 19.410 | 34.289 | 10.332 | 1.00 | 7.27 |
| ATOM | 705 | O | ARG | 87 | 18.170 | 34.129 | 10.237 | 1.00 | 8.33 |
| ATOM | 706 | N | ALA | 88 | 20.206 | 33.270 | 10.609 | 1.00 | 7.40 |
| ATOM | 707 | CA | ALA | 88 | 19.749 | 31.876 | 10.750 | 1.00 | 6.92 |
| ATOM | 708 | CB | ALA | 88 | 20.760 | 30.869 | 10.254 | 1.00 | 8.59 |
| ATOM | 709 | C | ALA | 88 | 19.373 | 31.526 | 12.165 | 1.00 | 7.06 |
| ATOM | 710 | O | ALA | 88 | 20.138 | 31.793 | 13.084 | 1.00 | 9.57 |
| ATOM | 711 | N | GLY | 89 | 18.191 | 30.953 | 12.332 | 1.00 | 6.44 |
| ATOM | 712 | CA | GLY | 89 | 17.673 | 30.494 | 13.592 | 1.00 | 6.74 |
| ATOM | 713 | C | GLY | 89 | 16.222 | 30.872 | 13.801 | 1.00 | 6.23 |
| ATOM | 714 | O | GLY | 89 | 15.451 | 31.150 | 12.853 | 1.00 | 6.32 |
| ATOM | 715 | N | TYR | 90 | 15.811 | 30.854 | 15.062 | 1.00 | 6.31 |
| ATOM | 716 | CA | TYR | 90 | 14.439 | 31.063 | 15.460 | 1.00 | 6.71 |
| ATOM | 717 | CB | TYR | 90 | 14.009 | 30.142 | 16.623 | 1.00 | 7.29 |
| ATOM | 718 | CG | TYR | 90 | 12.552 | 29.793 | 16.487 | 1.00 | 6.81 |
| ATOM | 719 | CD1 | TYR | 90 | 12.151 | 28.687 | 15.755 | 1.00 | 7.89 |
| ATOM | 720 | CE1 | TYR | 90 | 10.830 | 28.357 | 15.592 | 1.00 | 8.96 |
| ATOM | 721 | CD2 | TYR | 90 | 11.536 | 30.556 | 17.042 | 1.00 | 7.06 |
| ATOM | 722 | CE2 | TYR | 90 | 10.193 | 30.257 | 16.837 | 1.00 | 8.48 |
| ATOM | 723 | CZ | TYR | 90 | 9.854 | 29.145 | 16.124 | 1.00 | 9.10 |
| ATOM | 724 | OH | TYR | 90 | 8.555 | 28.790 | 15.864 | 1.00 | 13.43 |
| ATOM | 725 | C | TYR | 90 | 14.191 | 32.541 | 15.802 | 1.00 | 6.30 |
| ATOM | 726 | O | TYR | 90 | 14.986 | 33.184 | 16.491 | 1.00 | 6.78 |
| ATOM | 727 | N | TYR | 91 | 13.035 | 33.030 | 15.320 | 1.00 | 6.07 |
| ATOM | 728 | CA | TYR | 91 | 12.554 | 34.384 | 15.484 | 1.00 | 6.47 |
| ATOM | 729 | CB | TYR | 91 | 12.252 | 35.025 | 14.135 | 1.00 | 6.78 |
| ATOM | 730 | CG | TYR | 91 | 13.392 | 35.277 | 13.209 | 1.00 | 6.23 |
| ATOM | 731 | CD1 | TYR | 91 | 14.353 | 34.332 | 12.859 | 1.00 | 7.06 |
| ATOM | 732 | CE1 | TYR | 91 | 15.364 | 34.597 | 11.962 | 1.00 | 7.29 |
| ATOM | 733 | CD2 | TYR | 91 | 13.490 | 36.521 | 12.556 | 1.00 | 7.46 |
| ATOM | 734 | CE2 | TYR | 91 | 14.486 | 36.776 | 11.634 | 1.00 | 7.96 |
| ATOM | 735 | CZ | TYR | 91 | 15.428 | 35.826 | 11.343 | 1.00 | 6.61 |
| ATOM | 736 | OH | TYR | 91 | 16.438 | 36.143 | 10.440 | 1.00 | 8.55 |
| ATOM | 737 | C | TYR | 91 | 11.289 | 34.262 | 16.334 | 1.00 | 5.71 |
| ATOM | 738 | O | TYR | 91 | 10.273 | 33.788 | 15.843 | 1.00 | 6.82 |
| ATOM | 739 | N | LEU | 92 | 11.385 | 34.619 | 17.619 | 1.00 | 6.69 |
| ATOM | 740 | CA | LEU | 92 | 10.306 | 34.337 | 18.569 | 1.00 | 6.33 |
| ATOM | 741 | CB | LEU | 92 | 10.873 | 34.371 | 19.987 | 1.00 | 7.20 |
| ATOM | 742 | CG | LEU | 92 | 11.700 | 33.188 | 20.466 | 1.00 | 7.89 |
| ATOM | 743 | CD1 | LEU | 92 | 13.022 | 33.065 | 19.735 | 1.00 | 8.67 |
| ATOM | 744 | CD2 | LEU | 92 | 11.888 | 33.261 | 21.985 | 1.00 | 10.35 |
| ATOM | 745 | C | LEU | 92 | 9.151 | 35.317 | 18.471 | 1.00 | 6.37 |
| ATOM | 746 | O | LEU | 92 | 9.284 | 36.491 | 18.161 | 1.00 | 7.36 |
| ATOM | 747 | N | SER | 93 | 7.976 | 34.786 | 18.810 | 1.00 | 7.12 |
| ATOM | 748 | CA | SER | 93 | 6.805 | 35.605 | 19.060 | 1.00 | 7.59 |
| ATOM | 749 | CB | SER | 93 | 5.523 | 34.794 | 19.013 | 1.00 | 8.96 |
| ATOM | 750 | OG | SER | 93 | 5.410 | 33.894 | 20.074 | 1.00 | 11.17 |
| ATOM | 751 | C | SER | 93 | 6.928 | 36.224 | 20.443 | 1.00 | 7.83 |
| ATOM | 752 | O | SER | 93 | 7.728 | 35.807 | 21.266 | 1.00 | 8.58 |
| ATOM | 753 | N | ILE | 94 | 6.100 | 37.213 | 20.688 | 1.00 | 9.38 |
| ATOM | 754 | CA | ILE | 94 | 6.029 | 37.865 | 22.016 | 1.00 | 9.91 |
| ATOM | 755 | CB | ILE | 94 | 6.640 | 39.280 | 21.992 | 1.00 | 10.58 |
| ATOM | 756 | CG2 | ILE | 94 | 6.575 | 39.842 | 23.385 | 1.00 | 11.13 |
| ATOM | 757 | CG1 | ILE | 94 | 8.051 | 39.304 | 21.385 | 1.00 | 11.02 |
| ATOM | 758 | CD1 | ILE | 94 | 8.740 | 40.630 | 21.225 | 1.00 | 12.81 |
| ATOM | 759 | C | ILE | 94 | 4.550 | 37.947 | 22.376 | 1.00 | 9.85 |
| ATOM | 760 | O | ILE | 94 | 3.881 | 38.887 | 21.906 | 1.00 | 11.19 |
| ATOM | 761 | N | PRO | 95 | 4.006 | 36.948 | 23.046 | 1.00 | 10.93 |
| ATOM | 762 | CD | PRO | 95 | 4.693 | 35.749 | 23.520 | 1.00 | 12.42 |
| ATOM | 763 | CA | PRO | 95 | 2.560 | 36.878 | 23.342 | 1.00 | 12.23 |
| ATOM | 764 | CB | PRO | 95 | 2.394 | 35.670 | 24.234 | 1.00 | 13.83 |
| ATOM | 765 | CG | PRO | 95 | 3.555 | 34.807 | 23.867 | 1.00 | 13.55 |
| ATOM | 766 | C | PRO | 95 | 2.064 | 38.173 | 23.973 | 1.00 | 11.65 |
| ATOM | 767 | O | PRO | 95 | 2.791 | 38.800 | 24.780 | 1.00 | 12.20 |
| ATOM | 768 | N | GLY | 96 | 0.973 | 38.667 | 23.409 | 1.00 | 12.67 |
| ATOM | 769 | CA | GLY | 96 | 0.479 | 39.965 | 23.799 | 1.00 | 13.37 |
| ATOM | 770 | C | GLY | 96 | 0.933 | 41.142 | 22.976 | 1.00 | 12.23 |
| ATOM | 771 | O | GLY | 96 | 0.366 | 42.233 | 23.105 | 1.00 | 15.48 |
| ATOM | 772 | N | LYS | 97 | 1.992 | 40.975 | 22.189 | 1.00 | 12.23 |
| ATOM | 773 | CA | LYS | 97 | 2.637 | 42.048 | 21.444 | 1.00 | 11.30 |
| ATOM | 774 | CB | LYS | 97 | 3.947 | 42.410 | 22.187 | 1.00 | 12.24 |
| ATOM | 775 | CG | LYS | 97 | 3.784 | 42.828 | 23.625 | 1.00 | 13.52 |
| ATOM | 776 | CD | LYS | 97 | 3.087 | 44.147 | 23.758 | 1.00 | 15.77 |
| ATOM | 777 | CE | LYS | 97 | 3.019 | 44.549 | 25.226 | 1.00 | 18.45 |
| ATOM | 778 | NZ | LYS | 97 | 2.107 | 45.715 | 25.339 | 1.00 | 21.87 |
| ATOM | 779 | C | LYS | 97 | 2.977 | 41.783 | 19.977 | 1.00 | 10.88 |
| ATOM | 780 | O | LYS | 97 | 2.857 | 42.638 | 19.081 | 1.00 | 11.92 |
| ATOM | 781 | N | LYS | 98 | 3.453 | 40.583 | 19.691 | 1.00 | 10.38 |
| ATOM | 782 | CA | LYS | 98 | 3.913 | 40.149 | 18.374 | 1.00 | 9.59 |
| ATOM | 783 | CB | LYS | 98 | 5.442 | 40.215 | 18.332 | 1.00 | 12.06 |
| ATOM | 784 | CG | LYS | 98 | 6.093 | 39.618 | 17.105 | 1.00 | 11.30 |
| ATOM | 785 | CD | LYS | 98 | 7.604 | 39.879 | 17.116 | 1.00 | 10.99 |
| ATOM | 786 | CE | LYS | 98 | 8.338 | 39.370 | 15.908 | 1.00 | 10.76 |
| ATOM | 787 | NZ | LYS | 98 | 8.448 | 37.856 | 15.937 | 1.00 | 10.00 |
| ATOM | 788 | C | LYS | 98 | 3.462 | 38.723 | 18.145 | 1.00 | 9.52 |
| ATOM | 789 | O | LYS | 98 | 3.835 | 37.797 | 18.884 | 1.00 | 10.44 |
| ATOM | 790 | N | ALA | 99 | 2.674 | 38.529 | 17.108 | 1.00 | 8.69 |
| ATOM | 791 | CA | ALA | 99 | 2.095 | 37.221 | 16.865 | 1.00 | 8.30 |
| ATOM | 792 | CB | ALA | 99 | 0.771 | 37.440 | 16.107 | 1.00 | 11.01 |
| ATOM | 793 | C | ALA | 99 | 2.993 | 36.289 | 16.048 | 1.00 | 7.09 |
| ATOM | 794 | O | ALA | 99 | 3.091 | 35.086 | 16.291 | 1.00 | 8.34 |
| ATOM | 795 | N | VAL | 100 | 3.614 | 36.851 | 14.990 | 1.00 | 7.33 |
| ATOM | 796 | CA | VAL | 100 | 4.390 | 36.044 | 14.085 | 1.00 | 7.31 |
| ATOM | 797 | CB | VAL | 100 | 4.804 | 36.909 | 12.882 | 1.00 | 8.26 |
| ATOM | 798 | CG1 | VAL | 100 | 5.854 | 37.965 | 13.230 | 1.00 | 9.84 |
| ATOM | 799 | CG2 | VAL | 100 | 5.294 | 36.092 | 11.703 | 1.00 | 9.08 |
| ATOM | 800 | C | VAL | 100 | 5.636 | 35.463 | 14.759 | 1.00 | 6.77 |
| ATOM | 801 | O | VAL | 100 | 6.274 | 36.069 | 15.614 | 1.00 | 6.24 |
| ATOM | 802 | N | GLU | 101 | 6.039 | 34.294 | 14.261 | 1.00 | 5.93 |
| ATOM | 803 | CA | GLU | 101 | 7.275 | 33.647 | 14.643 | 1.00 | 5.89 |
| ATOM | 804 | CB | GLU | 101 | 7.164 | 32.758 | 15.864 | 1.00 | 6.83 |
| ATOM | 805 | CG | GLU | 101 | 6.194 | 31.599 | 15.714 | 1.00 | 9.01 |
| ATOM | 806 | CD | GLU | 101 | 5.993 | 30.900 | 17.058 | 1.00 | 11.15 |
| ATOM | 807 | OE1 | GLU | 101 | 4.957 | 31.171 | 17.727 | 1.00 | 11.35 |
| ATOM | 808 | OE2 | GLU | 101 | 6.839 | 30.033 | 17.396 | 1.00 | 16.56 |
| ATOM | 809 | C | GLU | 101 | 7.748 | 32.835 | 13.441 | 1.00 | 6.12 |
| ATOM | 810 | O | GLU | 101 | 6.989 | 32.513 | 12.532 | 1.00 | 6.44 |
| ATOM | 811 | N | SER | 102 | 9.047 | 32.564 | 13.384 | 1.00 | 6.02 |
| ATOM | 812 | CA | SER | 102 | 9.605 | 31.897 | 12.196 | 1.00 | 6.00 |
| ATOM | 813 | CB | SER | 102 | 9.725 | 32.906 | 11.058 | 1.00 | 6.77 |
| ATOM | 814 | OG | SER | 102 | 10.632 | 33.935 | 11.388 | 1.00 | 7.64 |
| ATOM | 815 | C | SER | 102 | 10.934 | 31.253 | 12.468 | 1.00 | 6.15 |
| ATOM | 816 | O | SER | 102 | 11.592 | 31.497 | 13.474 | 1.00 | 6.61 |
| ATOM | 817 | N | PHE | 103 | 11.367 | 30.433 | 11.517 | 1.00 | 5.73 |
| ATOM | 818 | CA | PHE | 103 | 12.670 | 29.792 | 11.506 | 1.00 | 5.88 |
| ATOM | 819 | CB | PHE | 103 | 12.521 | 28.294 | 11.756 | 1.00 | 6.37 |
| ATOM | 820 | CG | PHE | 103 | 13.795 | 27.476 | 11.663 | 1.00 | 6.21 |
| ATOM | 821 | CD1 | PHE | 103 | 14.723 | 27.499 | 12.686 | 1.00 | 7.53 |
| ATOM | 822 | CD2 | PHE | 103 | 14.034 | 26.650 | 10.562 | 1.00 | 7.03 |
| ATOM | 823 | CE1 | PHE | 103 | 15.866 | 26.713 | 12.620 | 1.00 | 7.78 |
| ATOM | 824 | CE2 | PHE | 103 | 15.167 | 25.887 | 10.483 | 1.00 | 6.93 |
| ATOM | 825 | CZ | PHE | 103 | 16.077 | 25.924 | 11.508 | 1.00 | 7.07 |
| ATOM | 826 | C | PHE | 103 | 13.319 | 30.018 | 10.149 | 1.00 | 5.84 |
| ATOM | 827 | O | PHE | 103 | 12.720 | 29.679 | 9.124 | 1.00 | 5.89 |
| ATOM | 828 | N | CYS | 104 | 14.505 | 30.596 | 10.128 | 1.00 | 5.38 |
| ATOM | 829 | CA | CYS | 104 | 15.244 | 30.888 | 8.907 | 1.00 | 5.34 |
| ATOM | 830 | CB | CYS | 104 | 15.669 | 32.368 | 8.935 | 1.00 | 6.38 |
| ATOM | 831 | SG | CYS | 104 | 16.693 | 32.902 | 7.544 | 1.00 | 7.14 |
| ATOM | 832 | C | CYS | 104 | 16.475 | 29.988 | 8.802 | 1.00 | 5.05 |
| ATOM | 833 | O | CYS | 104 | 17.202 | 29.801 | 9.794 | 1.00 | 5.55 |
| ATOM | 834 | N | TYR | 105 | 16.758 | 29.512 | 7.615 | 1.00 | 5.01 |
| ATOM | 835 | CA | TYR | 105 | 17.979 | 28.794 | 7.344 | 1.00 | 4.98 |
| ATOM | 836 | CB | TYR | 105 | 17.839 | 27.285 | 7.499 | 1.00 | 5.67 |
| ATOM | 837 | CG | TYR | 105 | 16.822 | 26.594 | 6.589 | 1.00 | 5.07 |
| ATOM | 838 | CD1 | TYR | 105 | 17.258 | 25.856 | 5.491 | 1.00 | 5.34 |
| ATOM | 839 | CE1 | TYR | 105 | 16.371 | 25.171 | 4.659 | 1.00 | 5.87 |
| ATOM | 840 | CD2 | TYR | 105 | 15.469 | 26.551 | 6.883 | 1.00 | 5.19 |
| ATOM | 841 | CE2 | TYR | 105 | 14.595 | 25.862 | 6.059 | 1.00 | 5.89 |
| ATOM | 842 | CZ | TYR | 105 | 15.045 | 25.169 | 4.966 | 1.00 | 5.38 |
| ATOM | 843 | OH | TYR | 105 | 14.141 | 24.495 | 4.169 | 1.00 | 6.39 |
| ATOM | 844 | C | TYR | 105 | 18.515 | 29.202 | 5.972 | 1.00 | 4.38 |
| ATOM | 845 | O | TYR | 105 | 17.778 | 29.622 | 5.072 | 1.00 | 5.26 |
| ATOM | 846 | N | LEU | 106 | 19.816 | 29.039 | 5.852 | 1.00 | 5.00 |
| ATOM | 847 | CA | LEU | 106 | 20.634 | 29.415 | 4.731 | 1.00 | 4.93 |
| ATOM | 848 | CB | LEU | 106 | 21.803 | 30.266 | 5.227 | 1.00 | 6.26 |
| ATOM | 849 | CG | LEU | 106 | 21.444 | 31.467 | 6.125 | 1.00 | 6.45 |
| ATOM | 850 | CD1 | LEU | 106 | 22.709 | 32.219 | 6.477 | 1.00 | 8.37 |
| ATOM | 851 | CD2 | LEU | 106 | 20.392 | 32.329 | 5.466 | 1.00 | 6.90 |
| ATOM | 852 | C | LEU | 106 | 21.121 | 28.188 | 3.948 | 1.00 | 4.77 |
| ATOM | 853 | O | LEU | 106 | 20.754 | 27.046 | 4.237 | 1.00 | 5.93 |
| ATOM | 854 | N | ASN | 107 | 21.991 | 28.473 | 2.983 | 1.00 | 4.96 |
| ATOM | 855 | CA | ASN | 107 | 22.654 | 27.437 | 2.187 | 1.00 | 5.23 |
| ATOM | 856 | CB | ASN | 107 | 23.803 | 28.147 | 1.434 | 1.00 | 5.83 |
| ATOM | 857 | CG | ASN | 107 | 24.424 | 27.280 | 0.353 | 1.00 | 5.32 |
| ATOM | 858 | OD1 | ASN | 107 | 24.420 | 26.044 | 0.396 | 1.00 | 5.91 |
| ATOM | 859 | ND2 | ASN | 107 | 24.989 | 27.988 | −0.626 | 1.00 | 6.49 |
| ATOM | 860 | C | ASN | 107 | 23.225 | 26.347 | 3.079 | 1.00 | 4.82 |
| ATOM | 861 | O | ASN | 107 | 24.097 | 26.593 | 3.937 | 1.00 | 5.62 |
| ATOM | 862 | N | PRO | 108 | 22.771 | 25.095 | 2.881 | 1.00 | 4.96 |
| ATOM | 863 | CD | PRO | 108 | 21.595 | 24.677 | 2.118 | 1.00 | 5.43 |
| ATOM | 864 | CA | PRO | 108 | 23.345 | 23.973 | 3.639 | 1.00 | 5.82 |
| ATOM | 865 | CB | PRO | 108 | 22.516 | 22.773 | 3.134 | 1.00 | 6.84 |
| ATOM | 866 | CG | PRO | 108 | 21.194 | 23.377 | 2.749 | 1.00 | 6.32 |
| ATOM | 867 | C | PRO | 108 | 24.846 | 23.763 | 3.444 | 1.00 | 5.93 |
| ATOM | 868 | O | PRO | 108 | 25.533 | 23.129 | 4.274 | 1.00 | 6.51 |
| ATOM | 869 | N | ASN | 109 | 25.397 | 24.277 | 2.353 | 1.00 | 5.53 |
| ATOM | 870 | CA | ASN | 109 | 26.823 | 24.180 | 2.080 | 1.00 | 5.84 |
| ATOM | 871 | CB | ASN | 109 | 27.130 | 24.369 | 0.598 | 1.00 | 6.10 |
| ATOM | 872 | CG | ASN | 109 | 26.633 | 23.186 | −0.200 | 1.00 | 5.51 |
| ATOM | 873 | OD1 | ASN | 109 | 26.690 | 22.020 | 0.209 | 1.00 | 7.43 |
| ATOM | 874 | ND2 | ASN | 109 | 26.165 | 23.491 | −1.405 | 1.00 | 7.14 |
| ATOM | 875 | C | ASN | 109 | 27.652 | 25.150 | 2.920 | 1.00 | 5.95 |
| ATOM | 876 | O | ASN | 109 | 28.863 | 25.063 | 2.911 | 1.00 | 6.60 |
| ATOM | 877 | N | PHE | 110 | 27.032 | 26.060 | 3.672 | 1.00 | 6.52 |
| ATOM | 878 | CA | PHE | 110 | 27.727 | 26.943 | 4.602 | 1.00 | 6.64 |
| ATOM | 879 | CB | PHE | 110 | 26.984 | 28.235 | 4.934 | 1.00 | 6.86 |
| ATOM | 880 | CG | PHE | 110 | 26.793 | 29.203 | 3.781 | 1.00 | 6.62 |
| ATOM | 881 | CD1 | PHE | 110 | 27.454 | 29.100 | 2.593 | 1.00 | 6.42 |
| ATOM | 882 | CD2 | PHE | 110 | 25.889 | 30.238 | 3.919 | 1.00 | 7.13 |
| ATOM | 883 | CE1 | PHE | 110 | 27.269 | 30.020 | 1.570 | 1.00 | 7.97 |
| ATOM | 884 | CE2 | PHE | 110 | 25.698 | 31.178 | 2.917 | 1.00 | 7.73 |
| ATOM | 885 | CZ | PHE | 110 | 26.380 | 31.064 | 1.723 | 1.00 | 7.68 |
| ATOM | 886 | C | PHE | 110 | 28.010 | 26.131 | 5.870 | 1.00 | 6.41 |
| ATOM | 887 | O | PHE | 110 | 27.334 | 26.222 | 6.890 | 1.00 | 9.08 |
| ATOM | 888 | N | THR | 111 | 29.047 | 25.319 | 5.795 | 1.00 | 7.20 |
| ATOM | 889 | CA | THR | 111 | 29.543 | 24.473 | 6.858 | 1.00 | 7.97 |
| ATOM | 890 | CB | THR | 111 | 29.986 | 23.114 | 6.240 | 1.00 | 9.61 |
| ATOM | 891 | OG1 | THR | 111 | 30.863 | 23.407 | 5.150 | 1.00 | 12.08 |
| ATOM | 892 | CG2 | THR | 111 | 28.831 | 22.332 | 5.660 | 1.00 | 11.21 |
| ATOM | 893 | C | THR | 111 | 30.719 | 25.173 | 7.535 | 1.00 | 8.55 |
| ATOM | 894 | O | THR | 111 | 31.262 | 26.161 | 7.039 | 1.00 | 8.43 |
| ATOM | 895 | N | PRO | 112 | 31.158 | 24.669 | 8.690 | 1.00 | 10.82 |
| ATOM | 896 | CD | PRO | 112 | 30.500 | 23.646 | 9.514 | 1.00 | 12.34 |
| ATOM | 897 | CA | PRO | 112 | 32.284 | 25.317 | 9.365 | 1.00 | 10.76 |
| ATOM | 898 | CB | PRO | 112 | 32.454 | 24.403 | 10.591 | 1.00 | 12.53 |
| ATOM | 899 | CG | PRO | 112 | 31.035 | 23.971 | 10.884 | 1.00 | 13.67 |
| ATOM | 900 | C | PRO | 112 | 33.560 | 25.453 | 8.553 | 1.00 | 10.13 |
| ATOM | 901 | O | PRO | 112 | 34.293 | 26.449 | 8.719 | 1.00 | 12.02 |
| ATOM | 902 | N | ASP | 113 | 33.764 | 24.577 | 7.586 | 1.00 | 10.28 |
| ATOM | 903 | CA | ASP | 113 | 34.960 | 24.594 | 6.754 | 1.00 | 11.85 |
| ATOM | 904 | CB | ASP | 113 | 35.418 | 23.179 | 6.354 | 1.00 | 14.15 |
| ATOM | 905 | CG | ASP | 113 | 34.450 | 22.335 | 5.556 | 1.00 | 16.88 |
| ATOM | 906 | OD1 | ASP | 113 | 34.697 | 21.122 | 5.265 | 1.00 | 17.94 |
| ATOM | 907 | OD2 | ASP | 113 | 33.431 | 22.903 | 5.120 | 1.00 | 18.33 |
| ATOM | 908 | C | ASP | 113 | 34.779 | 25.485 | 5.516 | 1.00 | 10.04 |
| ATOM | 909 | O | ASP | 113 | 35.695 | 25.568 | 4.674 | 1.00 | 11.29 |
| ATOM | 910 | N | HIS | 114 | 33.614 | 26.058 | 5.256 | 1.00 | 8.19 |
| ATOM | 911 | CA | HIS | 114 | 33.388 | 26.853 | 4.066 | 1.00 | 7.72 |
| ATOM | 912 | CB | HIS | 114 | 31.924 | 27.234 | 3.945 | 1.00 | 8.19 |
| ATOM | 913 | CG | HIS | 114 | 31.514 | 27.896 | 2.661 | 1.00 | 7.35 |
| ATOM | 914 | CD2 | HIS | 114 | 30.786 | 27.365 | 1.634 | 1.00 | 7.63 |
| ATOM | 915 | ND1 | HIS | 114 | 31.853 | 29.194 | 2.350 | 1.00 | 7.74 |
| ATOM | 916 | CE1 | HIS | 114 | 31.374 | 29.423 | 1.125 | 1.00 | 8.63 |
| ATOM | 917 | NE2 | HIS | 114 | 30.698 | 28.385 | 0.701 | 1.00 | 8.22 |
| ATOM | 918 | C | HIS | 114 | 34.263 | 28.078 | 4.069 | 1.00 | 7.89 |
| ATOM | 919 | O | HIS | 114 | 34.363 | 28.705 | 5.126 | 1.00 | 8.23 |
| ATOM | 920 | N | PRO | 115 | 34.877 | 28.447 | 2.947 | 1.00 | 8.07 |
| ATOM | 921 | CD | PRO | 115 | 34.805 | 27.853 | 1.595 | 1.00 | 9.44 |
| ATOM | 922 | CA | PRO | 115 | 35.791 | 29.586 | 2.989 | 1.00 | 7.97 |
| ATOM | 923 | CB | PRO | 115 | 36.432 | 29.638 | 1.603 | 1.00 | 10.29 |
| ATOM | 924 | CG | PRO | 115 | 36.058 | 28.363 | 0.935 | 1.00 | 16.51 |
| ATOM | 925 | C | PRO | 115 | 35.213 | 30.906 | 3.451 | 1.00 | 8.16 |
| ATOM | 926 | O | PRO | 115 | 35.957 | 31.749 | 4.033 | 1.00 | 8.89 |
| ATOM | 927 | N | ARG | 116 | 33.935 | 31.156 | 3.245 | 1.00 | 7.57 |
| ATOM | 928 | CA | ARG | 116 | 33.282 | 32.366 | 3.687 | 1.00 | 8.05 |
| ATOM | 929 | CB | ARG | 116 | 32.055 | 32.649 | 2.831 | 1.00 | 9.75 |
| ATOM | 930 | CG | ARG | 116 | 32.363 | 33.041 | 1.398 | 1.00 | 11.43 |
| ATOM | 931 | CD | ARG | 116 | 33.024 | 34.444 | 1.316 | 1.00 | 17.12 |
| ATOM | 932 | NE | ARG | 116 | 32.909 | 34.976 | −0.046 | 1.00 | 18.64 |
| ATOM | 933 | CZ | ARG | 116 | 33.267 | 36.202 | −0.332 | 1.00 | 16.55 |
| ATOM | 934 | NH1 | ARG | 116 | 33.732 | 37.005 | 0.626 | 1.00 | 18.90 |
| ATOM | 935 | NH2 | ARG | 116 | 33.158 | 36.628 | −1.550 | 1.00 | 19.38 |
| ATOM | 936 | C | ARG | 116 | 32.955 | 32.353 | 5.168 | 1.00 | 8.18 |
| ATOM | 937 | O | ARG | 116 | 32.905 | 33.426 | 5.807 | 1.00 | 10.55 |
| ATOM | 938 | N | ILE | 117 | 32.701 | 31.161 | 5.702 | 1.00 | 7.91 |
| ATOM | 939 | CA | ILE | 117 | 32.507 | 30.993 | 7.134 | 1.00 | 8.74 |
| ATOM | 940 | CB | ILE | 117 | 31.884 | 29.617 | 7.425 | 1.00 | 8.27 |
| ATOM | 941 | CG2 | ILE | 117 | 31.909 | 29.337 | 8.920 | 1.00 | 10.79 |
| ATOM | 942 | CG1 | ILE | 117 | 30.482 | 29.510 | 6.798 | 1.00 | 7.78 |
| ATOM | 943 | CD1 | ILE | 117 | 29.478 | 30.458 | 7.376 | 1.00 | 9.72 |
| ATOM | 944 | C | ILE | 117 | 33.863 | 31.170 | 7.818 | 1.00 | 9.52 |
| ATOM | 945 | O | ILE | 117 | 33.956 | 31.893 | 8.820 | 1.00 | 10.02 |
| ATOM | 946 | N | GLN | 118 | 34.936 | 30.601 | 7.248 | 1.00 | 9.60 |
| ATOM | 947 | CA | GLN | 118 | 36.264 | 30.823 | 7.814 | 1.00 | 9.55 |
| ATOM | 948 | CB | GLN | 118 | 37.287 | 29.975 | 7.066 | 1.00 | 11.20 |
| ATOM | 949 | CG | GLN | 118 | 37.163 | 28.475 | 7.285 | 1.00 | 11.87 |
| ATOM | 950 | CD | GLN | 118 | 37.706 | 28.064 | 8.628 | 1.00 | 15.84 |
| ATOM | 951 | OE1 | GLN | 118 | 38.799 | 28.509 | 9.021 | 1.00 | 16.95 |
| ATOM | 952 | NE2 | GLN | 118 | 36.968 | 27.189 | 9.314 | 1.00 | 18.58 |
| ATOM | 953 | C | GLN | 118 | 36.624 | 32.301 | 7.782 | 1.00 | 10.34 |
| ATOM | 954 | O | GLN | 118 | 37.209 | 32.780 | 8.779 | 1.00 | 12.18 |
| ATOM | 955 | N | ALA | 119 | 36.307 | 33.008 | 6.719 | 1.00 | 10.58 |
| ATOM | 956 | CA | ALA | 119 | 36.652 | 34.437 | 6.577 | 1.00 | 11.17 |
| ATOM | 957 | CB | ALA | 119 | 36.675 | 34.884 | 5.141 | 1.00 | 11.90 |
| ATOM | 958 | C | ALA | 119 | 35.766 | 35.340 | 7.417 | 1.00 | 11.81 |
| ATOM | 959 | O | ALA | 119 | 35.975 | 36.547 | 7.593 | 1.00 | 13.51 |
| ATOM | 960 | N | LYS | 120 | 34.677 | 34.792 | 7.926 | 1.00 | 11.51 |
| ATOM | 961 | CA | LYS | 120 | 33.697 | 35.524 | 8.728 | 1.00 | 11.83 |
| ATOM | 962 | CB | LYS | 120 | 34.314 | 36.156 | 9.981 | 1.00 | 16.43 |
| ATOM | 963 | CG | LYS | 120 | 35.156 | 35.192 | 10.773 | 1.00 | 20.21 |
| ATOM | 964 | CD | LYS | 120 | 34.452 | 34.007 | 11.336 | 1.00 | 25.82 |
| ATOM | 965 | CE | LYS | 120 | 35.422 | 33.080 | 12.083 | 1.00 | 26.48 |
| ATOM | 966 | NZ | LYS | 120 | 36.364 | 32.304 | 11.194 | 1.00 | 23.67 |
| ATOM | 967 | C | LYS | 120 | 33.033 | 36.623 | 7.910 | 1.00 | 12.07 |
| ATOM | 968 | O | LYS | 120 | 32.701 | 37.698 | 8.413 | 1.00 | 13.32 |
| ATOM | 969 | N | THR | 121 | 32.773 | 36.290 | 6.647 | 1.00 | 11.59 |
| ATOM | 970 | CA | THR | 121 | 32.162 | 37.294 | 5.783 | 1.00 | 11.41 |
| ATOM | 971 | CB | THR | 121 | 32.158 | 36.758 | 4.336 | 1.00 | 11.74 |
| ATOM | 972 | OG1 | THR | 121 | 33.515 | 36.460 | 3.947 | 1.00 | 14.18 |
| ATOM | 973 | CG2 | THR | 121 | 31.617 | 37.825 | 3.394 | 1.00 | 13.26 |
| ATOM | 974 | C | THR | 121 | 30.742 | 37.599 | 6.199 | 1.00 | 10.30 |
| ATOM | 975 | O | THR | 121 | 30.016 | 36.627 | 6.442 | 1.00 | 9.38 |
| ATOM | 976 | N | PRO | 122 | 30.343 | 38.869 | 6.255 | 1.00 | 11.24 |
| ATOM | 977 | CD | PRO | 122 | 31.175 | 40.087 | 6.162 | 1.00 | 13.40 |
| ATOM | 978 | CA | PRO | 122 | 28.938 | 39.176 | 6.605 | 1.00 | 10.67 |
| ATOM | 979 | CB | PRO | 122 | 28.883 | 40.676 | 6.374 | 1.00 | 13.30 |
| ATOM | 980 | CG | PRO | 122 | 30.251 | 41.155 | 6.684 | 1.00 | 14.04 |
| ATOM | 981 | C | PRO | 122 | 27.933 | 38.454 | 5.721 | 1.00 | 8.90 |
| ATOM | 982 | O | PRO | 122 | 28.224 | 38.174 | 4.567 | 1.00 | 9.02 |
| ATOM | 983 | N | THR | 123 | 26.810 | 38.105 | 6.329 | 1.00 | 9.47 |
| ATOM | 984 | CA | THR | 123 | 25.660 | 37.447 | 5.729 | 1.00 | 8.42 |
| ATOM | 985 | CB | THR | 123 | 25.112 | 38.105 | 4.455 | 1.00 | 9.93 |
| ATOM | 986 | OG1 | THR | 123 | 25.929 | 37.810 | 3.314 | 1.00 | 10.18 |
| ATOM | 987 | CG2 | THR | 123 | 25.024 | 39.629 | 4.512 | 1.00 | 13.04 |
| ATOM | 988 | C | THR | 123 | 25.807 | 35.938 | 5.560 | 1.00 | 8.70 |
| ATOM | 989 | O | THR | 123 | 24.815 | 35.273 | 5.198 | 1.00 | 9.15 |
| ATOM | 990 | N | HIS | 124 | 26.994 | 35.395 | 5.833 | 1.00 | 8.34 |
| ATOM | 991 | CA | HIS | 124 | 27.189 | 33.957 | 5.798 | 1.00 | 7.64 |
| ATOM | 992 | CB | HIS | 124 | 28.524 | 33.584 | 5.149 | 1.00 | 7.45 |
| ATOM | 993 | CG | HIS | 124 | 28.726 | 34.113 | 3.780 | 1.00 | 7.00 |
| ATOM | 994 | CD2 | HIS | 124 | 28.755 | 33.507 | 2.577 | 1.00 | 7.26 |
| ATOM | 995 | ND1 | HIS | 124 | 28.998 | 35.449 | 3.557 | 1.00 | 7.88 |
| ATOM | 996 | CE1 | HIS | 124 | 29.202 | 35.607 | 2.261 | 1.00 | 8.29 |
| ATOM | 997 | NE2 | HIS | 124 | 29.032 | 34.478 | 1.631 | 1.00 | 7.93 |
| ATOM | 998 | C | HIS | 124 | 27.170 | 33.387 | 7.206 | 1.00 | 8.19 |
| ATOM | 999 | O | HIS | 124 | 27.888 | 33.913 | 8.056 | 1.00 | 9.17 |
| ATOM | 1000 | N | GLU | 125 | 26.413 | 32.313 | 7.455 | 1.00 | 7.36 |
| ATOM | 1001 | CA | GLU | 125 | 26.337 | 31.694 | 8.776 | 1.00 | 7.74 |
| ATOM | 1002 | CB | GLU | 125 | 25.166 | 32.230 | 9.604 | 1.00 | 8.77 |
| ATOM | 1003 | CG | GLU | 125 | 25.164 | 33.714 | 9.832 | 1.00 | 9.78 |
| ATOM | 1004 | CD | GLU | 125 | 23.885 | 34.235 | 10.445 | 1.00 | 9.41 |
| ATOM | 1005 | OE1 | GLU | 125 | 23.057 | 33.439 | 10.972 | 1.00 | 12.02 |
| ATOM | 1006 | OE2 | GLU | 125 | 23.791 | 35.476 | 10.373 | 1.00 | 12.25 |
| ATOM | 1007 | C | GLU | 125 | 26.118 | 30.196 | 8.567 | 1.00 | 7.57 |
| ATOM | 1008 | O | GLU | 125 | 25.581 | 29.787 | 7.521 | 1.00 | 8.65 |
| ATOM | 1009 | N | VAL | 126 | 26.505 | 29.398 | 9.557 | 1.00 | 7.88 |
| ATOM | 1010 | CA | VAL | 126 | 26.219 | 27.969 | 9.636 | 1.00 | 7.38 |
| ATOM | 1011 | CB | VAL | 126 | 27.281 | 27.246 | 10.488 | 1.00 | 8.22 |
| ATOM | 1012 | CG1 | VAL | 126 | 26.979 | 25.754 | 10.561 | 1.00 | 9.54 |
| ATOM | 1013 | CG2 | VAL | 126 | 28.686 | 27.497 | 9.941 | 1.00 | 9.36 |
| ATOM | 1014 | C | VAL | 126 | 24.843 | 27.785 | 10.272 | 1.00 | 7.39 |
| ATOM | 1015 | O | VAL | 126 | 24.564 | 28.311 | 11.344 | 1.00 | 8.29 |
| ATOM | 1016 | N | ASN | 127 | 23.951 | 27.081 | 9.554 | 1.00 | 6.96 |
| ATOM | 1017 | CA | ASN | 127 | 22.618 | 26.839 | 10.081 | 1.00 | 6.87 |
| ATOM | 1018 | CB | ASN | 127 | 21.832 | 25.948 | 9.120 | 1.00 | 6.56 |
| ATOM | 1019 | CG | ASN | 127 | 21.515 | 26.650 | 7.803 | 1.00 | 6.11 |
| ATOM | 1020 | OD1 | ASN | 127 | 21.362 | 27.877 | 7.793 | 1.00 | 7.19 |
| ATOM | 1021 | ND2 | ASN | 127 | 21.360 | 25.859 | 6.758 | 1.00 | 7.23 |
| ATOM | 1022 | C | ASN | 127 | 22.624 | 26.173 | 11.447 | 1.00 | 7.20 |
| ATOM | 1023 | O | ASN | 127 | 23.503 | 25.353 | 11.763 | 1.00 | 8.68 |
| ATOM | 1024 | N | VAL | 128 | 21.596 | 26.511 | 12.239 | 1.00 | 7.13 |
| ATOM | 1025 | CA | VAL | 128 | 21.284 | 25.849 | 13.501 | 1.00 | 7.06 |
| ATOM | 1026 | CB | VAL | 128 | 21.172 | 26.848 | 14.557 | 1.00 | 8.77 |
| ATOM | 1027 | CG1 | VAL | 128 | 22.508 | 27.567 | 14.525 | 1.00 | 10.68 |
| ATOM | 1028 | CG2 | VAL | 128 | 19.986 | 27.774 | 14.501 | 1.00 | 9.46 |
| ATOM | 1029 | C | VAL | 123 | 20.032 | 25.032 | 13.329 | 1.00 | 6.59 |
| ATOM | 1030 | O | VAL | 128 | 19.101 | 25.426 | 12.617 | 1.00 | 8.06 |
| ATOM | 1031 | N | TRP | 129 | 19.977 | 23.861 | 13.963 | 1.00 | 7.79 |
| ATOM | 1032 | CA | TRP | 129 | 18.884 | 22.914 | 13.812 | 1.00 | 7.98 |
| ATOM | 1033 | CB | TRP | 129 | 19.316 | 21.709 | 12.955 | 1.00 | 7.99 |
| ATOM | 1034 | CG | TRP | 129 | 19.671 | 22.145 | 11.553 | 1.00 | 7.47 |
| ATOM | 1035 | CD2 | TRP | 129 | 18.743 | 22.448 | 10.496 | 1.00 | 6.77 |
| ATOM | 1036 | CE2 | TRP | 129 | 19.486 | 22.838 | 9.368 | 1.00 | 6.74 |
| ATOM | 1037 | CE3 | TRP | 129 | 17.353 | 22.428 | 10.400 | 1.00 | 6.42 |
| ATOM | 1038 | CD1 | TRP | 129 | 20.887 | 22.349 | 11.033 | 1.00 | 8.81 |
| ATOM | 1039 | NE1 | TRP | 129 | 20.809 | 22.765 | 9.721 | 1.00 | 8.43 |
| ATOM | 1040 | CZ2 | TRP | 129 | 18.902 | 23.196 | 8.160 | 1.00 | 6.63 |
| ATOM | 1041 | CZ3 | TRP | 129 | 16.754 | 22.796 | 9.207 | 1.00 | 7.42 |
| ATOM | 1042 | CH2 | TRP | 129 | 17.553 | 23.187 | 8.120 | 1.00 | 6.84 |
| ATOM | 1043 | C | TRP | 129 | 18.434 | 22.394 | 15.187 | 1.00 | 8.10 |
| ATOM | 1044 | O | TRP | 129 | 19.266 | 22.268 | 16.108 | 1.00 | 9.20 |
| ATOM | 1045 | N | PRO | 130 | 17.158 | 22.038 | 15.321 | 1.00 | 9.10 |
| ATOM | 1046 | CD | PRO | 130 | 16.078 | 22.208 | 14.338 | 1.00 | 11.04 |
| ATOM | 1047 | CA | PRO | 130 | 16.684 | 21.410 | 16.563 | 1.00 | 10.04 |
| ATOM | 1048 | CB | PRO | 130 | 15.190 | 21.366 | 16.377 | 1.00 | 12.74 |
| ATOM | 1049 | CG | PRO | 130 | 14.987 | 21.348 | 14.930 | 1.00 | 12.90 |
| ATOM | 1050 | C | PRO | 130 | 17.295 | 20.019 | 16.674 | 1.00 | 10.76 |
| ATOM | 1051 | O | PRO | 130 | 17.902 | 19.473 | 15.742 | 1.00 | 10.41 |
| ATOM | 1052 | N | ASP | 131 | 17.139 | 19.412 | 17.840 | 1.00 | 12.19 |
| ATOM | 1053 | CA | ASP | 131 | 17.611 | 18.063 | 18.062 | 1.00 | 13.83 |
| ATOM | 1054 | CB | ASP | 131 | 17.311 | 17.695 | 19.499 | 1.00 | 20.75 |
| ATOM | 1055 | CG | ASP | 131 | 18.290 | 18.307 | 20.474 | 1.00 | 30.05 |
| ATOM | 1056 | OD1 | ASP | 131 | 19.218 | 19.075 | 20.122 | 1.00 | 41.54 |
| ATOM | 1057 | OD2 | ASP | 131 | 18.111 | 17.974 | 21.678 | 1.00 | 44.79 |
| ATOM | 1058 | C | ASP | 131 | 16.864 | 17.077 | 17.175 | 1.00 | 12.28 |
| ATOM | 1059 | O | ASP | 131 | 15.643 | 17.091 | 17.104 | 1.00 | 11.37 |
| ATOM | 1060 | N | GLU | 132 | 17.632 | 16.204 | 16.561 | 1.00 | 11.37 |
| ATOM | 1061 | CA | GLU | 132 | 17.032 | 15.236 | 15.660 | 1.00 | 12.37 |
| ATOM | 1062 | CB | GLU | 132 | 18.140 | 14.416 | 14.986 | 1.00 | 14.40 |
| ATOM | 1063 | CG | GLU | 132 | 17.667 | 13.221 | 14.169 | 1.00 | 14.02 |
| ATOM | 1064 | CD | GLU | 132 | 17.004 | 13.617 | 12.890 | 1.00 | 14.20 |
| ATOM | 1065 | OE1 | GLU | 132 | 17.327 | 14.733 | 12.391 | 1.00 | 13.96 |
| ATOM | 1066 | OE2 | GLU | 132 | 16.222 | 12.787 | 12.395 | 1.00 | 14.71 |
| ATOM | 1067 | C | GLU | 132 | 16.028 | 14.309 | 16.327 | 1.00 | 13.05 |
| ATOM | 1068 | O | GLU | 132 | 15.022 | 13.953 | 15.730 | 1.00 | 13.21 |
| ATOM | 1069 | N | THR | 133 | 16.283 | 13.922 | 17.576 | 1.00 | 15.02 |
| ATOM | 1070 | CA | THR | 133 | 15.337 | 13.065 | 18.267 | 1.00 | 17.13 |
| ATOM | 1071 | CB | THR | 133 | 15.911 | 12.663 | 19.645 | 1.00 | 19.10 |
| ATOM | 1072 | OG1 | THR | 133 | 16.214 | 13.817 | 20.436 | 1.00 | 30.56 |
| ATOM | 1073 | CG2 | THR | 133 | 17.200 | 11.914 | 19.407 | 1.00 | 21.80 |
| ATOM | 1074 | C | THR | 133 | 14.003 | 13.738 | 18.504 | 1.00 | 14.81 |
| ATOM | 1075 | O | THR | 133 | 12.976 | 13.052 | 18.539 | 1.00 | 18.31 |
| ATOM | 1076 | N | LYS | 134 | 13.992 | 15.065 | 18.681 | 1.00 | 14.08 |
| ATOM | 1077 | CA | LYS | 134 | 12.738 | 15.763 | 18.943 | 1.00 | 14.05 |
| ATOM | 1078 | CB | LYS | 134 | 13.028 | 17.072 | 19.683 | 1.00 | 16.10 |
| ATOM | 1079 | CG | LYS | 134 | 13.566 | 16.783 | 21.105 | 1.00 | 21.50 |
| ATOM | 1080 | CD | LYS | 134 | 13.739 | 18.052 | 21.912 | 1.00 | 24.72 |
| ATOM | 1081 | CE | LYS | 134 | 13.962 | 17.851 | 23.411 | 1.00 | 29.07 |
| ATOM | 1082 | NZ | LYS | 134 | 15.380 | 17.498 | 23.673 | 1.00 | 35.66 |
| ATOM | 1083 | C | LYS | 134 | 11.982 | 16.084 | 17.673 | 1.00 | 11.72 |
| ATOM | 1084 | O | LYS | 134 | 10.764 | 16.268 | 17.697 | 1.00 | 12.75 |
| ATOM | 1085 | N | HIS | 135 | 12.768 | 16.210 | 16.602 | 1.00 | 10.34 |
| ATOM | 1086 | CA | HIS | 135 | 12.229 | 16.559 | 15.278 | 1.00 | 9.70 |
| ATOM | 1087 | CB | HIS | 135 | 12.568 | 18.014 | 14.927 | 1.00 | 10.46 |
| ATOM | 1088 | CG | HIS | 135 | 11.859 | 18.971 | 15.855 | 1.00 | 11.60 |
| ATOM | 1089 | CD2 | HIS | 135 | 10.625 | 19.519 | 15.664 | 1.00 | 11.74 |
| ATOM | 1090 | ND1 | HIS | 135 | 12.334 | 19.405 | 17.077 | 1.00 | 13.42 |
| ATOM | 1091 | CE1 | HIS | 135 | 11.390 | 20.219 | 17.592 | 1.00 | 10.37 |
| ATOM | 1092 | NE2 | HIS | 135 | 10.358 | 20.261 | 16.753 | 1.00 | 14.68 |
| ATOM | 1093 | C | HIS | 135 | 12.753 | 15.614 | 14.207 | 1.00 | 9.49 |
| ATOM | 1094 | O | HIS | 135 | 13.491 | 16.005 | 13.302 | 1.00 | 8.95 |
| ATOM | 1095 | N | PRO | 136 | 12.410 | 14.336 | 14.298 | 1.00 | 10.29 |
| ATOM | 1096 | CD | PRO | 136 | 11.539 | 13.714 | 15.288 | 1.00 | 12.71 |
| ATOM | 1097 | CA | PRO | 136 | 12.996 | 13.348 | 13.367 | 1.00 | 10.63 |
| ATOM | 1098 | CB | PRO | 136 | 12.331 | 12.025 | 13.738 | 1.00 | 13.20 |
| ATOM | 1099 | CG | PRO | 136 | 11.373 | 12.308 | 14.821 | 1.00 | 15.42 |
| ATOM | 1100 | C | PRO | 136 | 12.727 | 13.656 | 11.902 | 1.00 | 9.37 |
| ATOM | 1101 | O | PRO | 136 | 11.583 | 13.947 | 11.540 | 1.00 | 10.11 |
| ATOM | 1102 | N | GLY | 137 | 13.801 | 13.643 | 11.131 | 1.00 | 9.15 |
| ATOM | 1103 | CA | GLY | 137 | 13.642 | 13.906 | 9.700 | 1.00 | 8.92 |
| ATOM | 1104 | C | GLY | 137 | 13.568 | 15.351 | 9.281 | 1.00 | 8.41 |
| ATOM | 1105 | O | GLY | 137 | 13.604 | 15.660 | 8.076 | 1.00 | 8.62 |
| ATOM | 1106 | N | PHE | 138 | 13.438 | 16.290 | 10.230 | 1.00 | 7.77 |
| ATOM | 1107 | CA | PHE | 138 | 13.189 | 17.683 | 9.858 | 1.00 | 7.53 |
| ATOM | 1108 | CB | PHE | 138 | 12.791 | 18.533 | 11.069 | 1.00 | 9.20 |
| ATOM | 1109 | CG | PHE | 138 | 12.676 | 20.014 | 10.773 | 1.00 | 8.61 |
| ATOM | 1110 | CD1 | PHE | 138 | 11.623 | 20.502 | 9.999 | 1.00 | 8.98 |
| ATOM | 1111 | CD2 | PHE | 138 | 13.628 | 20.897 | 11.264 | 1.00 | 8.98 |
| ATOM | 1112 | CE1 | PHE | 138 | 11.598 | 21.878 | 9.728 | 1.00 | 9.14 |
| ATOM | 1113 | CE2 | PHE | 138 | 13.584 | 22.238 | 11.007 | 1.00 | 9.39 |
| ATOM | 1114 | CZ | PHE | 138 | 12.555 | 22.730 | 10.231 | 1.00 | 8.60 |
| ATOM | 1115 | C | PHE | 138 | 14.355 | 18.318 | 9.123 | 1.00 | 6.57 |
| ATOM | 1116 | O | PHE | 138 | 14.155 | 18.953 | 8.090 | 1.00 | 6.31 |
| ATOM | 1117 | N | GLN | 139 | 15.553 | 18.234 | 9.700 | 1.00 | 7.35 |
| ATOM | 1118 | CA | GLN | 139 | 16.718 | 18.827 | 9.045 | 1.00 | 7.49 |
| ATOM | 1119 | CB | GLN | 139 | 17.990 | 18.600 | 9.867 | 1.00 | 7.76 |
| ATOM | 1120 | CG | GLN | 139 | 19.211 | 19.123 | 9.164 | 1.00 | 8.03 |
| ATOM | 1121 | CD | GLN | 139 | 20.475 | 19.034 | 10.008 | 1.00 | 9.61 |
| ATOM | 1122 | OE1 | GLN | 139 | 20.452 | 18.629 | 11.194 | 1.00 | 11.81 |
| ATOM | 1123 | NE2 | GLN | 139 | 21.556 | 19.403 | 9.361 | 1.00 | 10.42 |
| ATOM | 1124 | C | GLN | 139 | 16.898 | 18.263 | 7.634 | 1.00 | 6.36 |
| ATOM | 1125 | O | GLN | 139 | 17.148 | 19.042 | 6.703 | 1.00 | 6.70 |
| ATOM | 1126 | N | ASP | 140 | 16.792 | 16.962 | 7.476 | 1.00 | 7.41 |
| ATOM | 1127 | CA | ASP | 140 | 16.966 | 16.359 | 6.153 | 1.00 | 8.02 |
| ATOM | 1128 | CB | ASP | 140 | 17.014 | 14.845 | 6.267 | 1.00 | 10.06 |
| ATOM | 1129 | CG | ASP | 140 | 18.185 | 14.414 | 7.143 | 1.00 | 12.56 |
| ATOM | 1130 | OD1 | ASP | 140 | 19.263 | 15.008 | 7.017 | 1.00 | 15.40 |
| ATOM | 1131 | OD2 | ASP | 140 | 18.010 | 13.419 | 7.863 | 1.00 | 17.99 |
| ATOM | 1132 | C | ASP | 140 | 15.903 | 16.836 | 5.173 | 1.00 | 7.03 |
| ATOM | 1133 | O | ASP | 140 | 16.195 | 17.125 | 4.012 | 1.00 | 7.37 |
| ATOM | 1134 | N | PHE | 141 | 14.649 | 16.886 | 5.632 | 1.00 | 6.76 |
| ATOM | 1135 | CA | PHE | 141 | 13.592 | 17.404 | 4.806 | 1.00 | 6.92 |
| ATOM | 1136 | CB | PHE | 141 | 12.241 | 17.315 | 5.525 | 1.00 | 8.28 |
| ATOM | 1137 | CG | PHE | 141 | 11.180 | 18.059 | 4.700 | 1.00 | 10.59 |
| ATOM | 1138 | CD1 | PHE | 141 | 10.649 | 17.398 | 3.585 | 1.00 | 12.85 |
| ATOM | 1139 | CD2 | PHE | 141 | 10.766 | 19.326 | 4.979 | 1.00 | 12.09 |
| ATOM | 1140 | CE1 | PHE | 141 | 9.773 | 18.045 | 2.730 | 1.00 | 14.79 |
| ATOM | 1141 | CE2 | PHE | 141 | 9.946 | 20.027 | 4.097 | 1.00 | 12.80 |
| ATOM | 1142 | CZ | PHE | 141 | 9.514 | 19.385 | 2.960 | 1.00 | 15.56 |
| ATOM | 1143 | C | PHE | 141 | 13.898 | 18.843 | 4.381 | 1.00 | 6.10 |
| ATOM | 1144 | O | PHE | 141 | 13.715 | 19.227 | 3.224 | 1.00 | 5.52 |
| ATOM | 1145 | N | ALA | 142 | 14.235 | 19.692 | 5.355 | 1.00 | 5.91 |
| ATOM | 1146 | CA | ALA | 142 | 14.436 | 21.113 | 5.109 | 1.00 | 6.07 |
| ATOM | 1147 | CB | ALA | 142 | 14.597 | 21.835 | 6.444 | 1.00 | 6.15 |
| ATOM | 1148 | C | ALA | 142 | 15.593 | 21.396 | 4.153 | 1.00 | 5.38 |
| ATOM | 1149 | O | ALA | 142 | 15.534 | 22.273 | 3.289 | 1.00 | 5.69 |
| ATOM | 1150 | N | GLU | 143 | 16.660 | 20.630 | 4.306 | 1.00 | 5.79 |
| ATOM | 1151 | CA | GLU | 143 | 17.811 | 20.759 | 3.404 | 1.00 | 5.89 |
| ATOM | 1152 | CB | GLU | 143 | 19.021 | 19.977 | 3.912 | 1.00 | 6.66 |
| ATOM | 1153 | CG | GLU | 143 | 19.647 | 20.589 | 5.171 | 1.00 | 6.49 |
| ATOM | 1154 | CD | GLU | 143 | 20.818 | 19.857 | 5.742 | 1.00 | 8.57 |
| ATOM | 1155 | OE1 | GLU | 143 | 20.986 | 18.661 | 5.468 | 1.00 | 15.50 |
| ATOM | 1156 | OE2 | GLU | 143 | 21.529 | 20.401 | 6.607 | 1.00 | 8.03 |
| ATOM | 1157 | C | GLU | 143 | 17.426 | 20.335 | 1.982 | 1.00 | 6.15 |
| ATOM | 1158 | O | GLU | 143 | 17.798 | 21.030 | 1.046 | 1.00 | 6.03 |
| ATOM | 1159 | N | GLN | 144 | 16.717 | 19.195 | 1.852 | 1.00 | 6.24 |
| ATOM | 1160 | CA | GLN | 144 | 16.249 | 18.793 | 0.519 | 1.00 | 5.81 |
| ATOM | 1161 | CB | GLN | 144 | 15.582 | 17.418 | 0.622 | 1.00 | 7.33 |
| ATOM | 1162 | CG | GLN | 144 | 15.034 | 16.900 | −0.718 | 1.00 | 9.38 |
| ATOM | 1163 | CD | GLN | 144 | 16.102 | 16.723 | −1.755 | 1.00 | 11.31 |
| ATOM | 1164 | OE1 | GLN | 144 | 16.165 | 17.336 | −2.852 | 1.00 | 15.13 |
| ATOM | 1165 | NE2 | GLN | 144 | 17.017 | 15.794 | −1.443 | 1.00 | 13.37 |
| ATOM | 1166 | C | GLN | 144 | 15.322 | 19.843 | −0.068 | 1.00 | 6.08 |
| ATOM | 1167 | O | GLN | 144 | 15.367 | 20.113 | −1.286 | 1.00 | 6.65 |
| ATOM | 1168 | N | TYR | 145 | 14.450 | 20.464 | 0.723 | 1.00 | 5.44 |
| ATOM | 1169 | CA | TYR | 145 | 13.552 | 21.492 | 0.249 | 1.00 | 5.36 |
| ATOM | 1170 | CB | TYR | 145 | 12.562 | 21.956 | 1.318 | 1.00 | 5.45 |
| ATOM | 1171 | CG | TYR | 145 | 11.718 | 23.094 | 0.785 | 1.00 | 5.34 |
| ATOM | 1172 | CD1 | TYR | 145 | 10.850 | 22.844 | −0.266 | 1.00 | 5.77 |
| ATOM | 1173 | CE1 | TYR | 145 | 10.113 | 23.856 | −0.832 | 1.00 | 5.40 |
| ATOM | 1174 | CD2 | TYR | 145 | 11.863 | 24.392 | 1.209 | 1.00 | 6.05 |
| ATOM | 1175 | CE2 | TYR | 145 | 11.128 | 25.428 | 0.632 | 1.00 | 5.52 |
| ATOM | 1176 | CZ | TYR | 145 | 10.260 | 25.146 | −0.376 | 1.00 | 4.90 |
| ATOM | 1177 | OH | TYR | 145 | 9.505 | 26.126 | −1.002 | 1.00 | 6.57 |
| ATOM | 1178 | C | TYR | 145 | 14.350 | 22.668 | −0.324 | 1.00 | 4.85 |
| ATOM | 1179 | O | TYR | 145 | 14.009 | 23.173 | −1.384 | 1.00 | 5.67 |
| ATOM | 1180 | N | TYR | 146 | 15.400 | 23.055 | 0.370 | 1.00 | 5.22 |
| ATOM | 1181 | CA | TYR | 146 | 16.294 | 24.134 | −0.108 | 1.00 | 5.07 |
| ATOM | 1182 | CB | TYR | 146 | 17.492 | 24.302 | 0.818 | 1.00 | 6.31 |
| ATOM | 1183 | CG | TYR | 146 | 18.356 | 25.533 | 0.554 | 1.00 | 5.63 |
| ATOM | 1184 | CD1 | TYR | 146 | 18.217 | 26.654 | 1.367 | 1.00 | 4.93 |
| ATOM | 1185 | CE1 | TYR | 146 | 18.975 | 27.804 | 1.191 | 1.00 | 4.66 |
| ATOM | 1186 | CD2 | TYR | 146 | 19.292 | 25.595 | −0.471 | 1.00 | 5.91 |
| ATOM | 1187 | CE2 | TYR | 146 | 20.050 | 26.728 | −0.673 | 1.00 | 5.60 |
| ATOM | 1188 | CZ | TYR | 146 | 19.892 | 27.816 | 0.151 | 1.00 | 5.33 |
| ATOM | 1189 | OH | TYR | 146 | 20.681 | 28.929 | 0.076 | 1.00 | 6.01 |
| ATOM | 1190 | C | TYR | 146 | 16.740 | 23.831 | −1.547 | 1.00 | 4.88 |
| ATOM | 1191 | O | TYR | 146 | 16.630 | 24.691 | −2.413 | 1.00 | 5.89 |
| ATOM | 1192 | N | TRP | 147 | 17.186 | 22.580 | −1.792 | 1.00 | 5.20 |
| ATOM | 1193 | CA | TRP | 147 | 17.650 | 22.244 | −3.129 | 1.00 | 5.95 |
| ATOM | 1194 | CB | TRP | 147 | 18.544 | 20.995 | −3.105 | 1.00 | 6.61 |
| ATOM | 1195 | CG | TRP | 147 | 19.730 | 21.259 | −2.221 | 1.00 | 6.71 |
| ATOM | 1196 | CD2 | TRP | 147 | 20.675 | 22.345 | −2.390 | 1.00 | 6.87 |
| ATOM | 1197 | CE2 | TRP | 147 | 21.598 | 22.227 | −1.334 | 1.00 | 7.71 |
| ATOM | 1198 | CE3 | TRP | 147 | 20.828 | 23.398 | −3.303 | 1.00 | 7.63 |
| ATOM | 1199 | CD1 | TRP | 147 | 20.127 | 20.576 | −1.120 | 1.00 | 7.42 |
| ATOM | 1200 | NE1 | TRP | 147 | 21.230 | 21.118 | −0.574 | 1.00 | 7.49 |
| ATOM | 1201 | CZ2 | TRP | 147 | 22.641 | 23.133 | −1.198 | 1.00 | 8.32 |
| ATOM | 1202 | CZ3 | TRP | 147 | 21.873 | 24.299 | −3.147 | 1.00 | 8.76 |
| ATOM | 1203 | CH2 | TRP | 147 | 22.774 | 24.162 | 2.079 | 1.00 | 8.40 |
| ATOM | 1204 | C | TRP | 147 | 16.514 | 22.124 | −4.123 | 1.00 | 5.56 |
| ATOM | 1205 | O | TRP | 147 | 16.697 | 22.484 | −5.295 | 1.00 | 6.90 |
| ATOM | 1206 | N | ASP | 148 | 15.333 | 21.663 | −3.736 | 1.00 | 5.62 |
| ATOM | 1207 | CA | ASP | 148 | 14.192 | 21.610 | −4.644 | 1.00 | 6.73 |
| ATOM | 1208 | CB | ASP | 148 | 13.006 | 20.892 | −3.990 | 1.00 | 7.41 |
| ATOM | 1209 | CG | ASP | 148 | 13.180 | 19.397 | −3.794 | 1.00 | 9.94 |
| ATOM | 1210 | OD1 | ASP | 148 | 14.107 | 18.824 | −4.385 | 1.00 | 11.03 |
| ATOM | 1211 | OD2 | ASP | 148 | 12.308 | 18.805 | −3.115 | 1.00 | 12.94 |
| ATOM | 1212 | C | ASP | 148 | 13.807 | 23.013 | −5.076 | 1.00 | 6.41 |
| ATOM | 1213 | O | ASP | 148 | 13.602 | 23.288 | −6.281 | 1.00 | 6.39 |
| ATOM | 1214 | N | VAL | 149 | 13.678 | 23.959 | −4.112 | 1.00 | 5.67 |
| ATOM | 1215 | CA | VAL | 149 | 13.287 | 25.330 | −4.472 | 1.00 | 5.45 |
| ATOM | 1216 | CB | VAL | 149 | 12.664 | 26.047 | −3.279 | 1.00 | 5.40 |
| ATOM | 1217 | CG1 | VAL | 149 | 13.656 | 26.460 | −2.202 | 1.00 | 5.95 |
| ATOM | 1218 | CG2 | VAL | 149 | 11.883 | 27.265 | −3.771 | 1.00 | 6.77 |
| ATOM | 1219 | C | VAL | 149 | 14.421 | 26.084 | −5.158 | 1.00 | 5.36 |
| ATOM | 1220 | O | VAL | 149 | 14.173 | 26.951 | −6.016 | 1.00 | 5.85 |
| ATOM | 1221 | N | PHE | 150 | 15.669 | 25.750 | −4.879 | 1.00 | 5.71 |
| ATOM | 1222 | CA | PHE | 150 | 16.795 | 26.267 | −5.635 | 1.00 | 5.05 |
| ATOM | 1223 | CB | PHE | 150 | 18.111 | 25.675 | −5.056 | 1.00 | 6.21 |
| ATOM | 1224 | CG | PHE | 150 | 19.374 | 26.154 | −5.764 | 1.00 | 6.04 |
| ATOM | 1225 | CD1 | PHE | 150 | 20.158 | 27.164 | −5.267 | 1.00 | 7.32 |
| ATOM | 1226 | CD2 | PHE | 150 | 19.840 | 25.565 | −6.911 | 1.00 | 7.41 |
| ATOM | 1227 | CE1 | PHE | 150 | 21.279 | 27.602 | −5.932 | 1.00 | 7.77 |
| ATOM | 1228 | CE2 | PHE | 150 | 20.926 | 26.005 | −7.649 | 1.00 | 8.49 |
| ATOM | 1229 | CZ | PHE | 150 | 21.686 | 27.017 | −7.119 | 1.00 | 7.48 |
| ATOM | 1230 | C | PHE | 150 | 16.616 | 25.887 | −7.102 | 1.00 | 5.89 |
| ATOM | 1231 | O | PHE | 150 | 16.841 | 26.726 | −8.004 | 1.00 | 6.52 |
| ATOM | 1232 | N | GLY | 151 | 16.276 | 24.637 | −7.351 | 1.00 | 6.70 |
| ATOM | 1233 | CA | GLY | 151 | 16.124 | 24.168 | −8.744 | 1.00 | 6.74 |
| ATOM | 1234 | C | GLY | 151 | 15.012 | 24.887 | −9.476 | 1.00 | 6.45 |
| ATOM | 1235 | O | GLY | 151 | 15.150 | 25.293 | −10.619 | 1.00 | 7.27 |
| ATOM | 1236 | N | LEU | 152 | 13.867 | 25.094 | −8.825 | 1.00 | 6.82 |
| ATOM | 1237 | CA | LEU | 152 | 12.787 | 25.878 | −9.404 | 1.00 | 6.24 |
| ATOM | 1238 | CB | LEU | 152 | 11.582 | 25.881 | −8.452 | 1.00 | 6.29 |
| ATOM | 1239 | CG | LEU | 152 | 10.452 | 26.859 | −8.828 | 1.00 | 7.25 |
| ATOM | 1240 | CD1 | LEU | 152 | 9.864 | 26.532 | −10.214 | 1.00 | 9.19 |
| ATOM | 1241 | CD2 | LEU | 152 | 9.358 | 26.832 | −7.763 | 1.00 | 7.05 |
| ATOM | 1242 | C | LEU | 152 | 13.269 | 27.298 | −9.669 | 1.00 | 6.42 |
| ATOM | 1243 | O | LEU | 152 | 13.015 | 27.864 | −10.743 | 1.00 | 7.31 |
| ATOM | 1244 | N | SER | 153 | 13.998 | 27.874 | −8.702 | 1.00 | 5.98 |
| ATOM | 1245 | CA | SER | 153 | 14.442 | 29.262 | −8.845 | 1.00 | 6.16 |
| ATOM | 1246 | CB | SER | 153 | 15.068 | 29.742 | −7.541 | 1.00 | 6.44 |
| ATOM | 1247 | OG | SER | 153 | 14.090 | 29.755 | −6.522 | 1.00 | 6.18 |
| ATOM | 1248 | C | SER | 153 | 15.424 | 29.413 | −10.000 | 1.00 | 6.14 |
| ATOM | 1249 | O | SER | 153 | 15.368 | 30.420 | −10.740 | 1.00 | 6.53 |
| ATOM | 1250 | N | SER | 154 | 16.284 | 28.436 | −10.217 | 1.00 | 6.45 |
| ATOM | 1251 | CA | SER | 154 | 17.220 | 28.495 | −11.340 | 1.00 | 7.39 |
| ATOM | 1252 | CB | SER | 154 | 18.126 | 27.271 | −11.278 | 1.00 | 8.84 |
| ATOM | 1253 | OG | SER | 154 | 18.981 | 27.292 | 10.189 | 1.00 | 13.14 |
| ATOM | 1254 | C | SER | 154 | 16.418 | 28.472 | −12.643 | 1.00 | 7.57 |
| ATOM | 1255 | O | SER | 154 | 16.742 | 29.253 | −13.563 | 1.00 | 8.31 |
| ATOM | 1256 | N | ALA | 155 | 15.408 | 27.630 | −12.737 | 1.00 | 6.91 |
| ATOM | 1257 | CA | ALA | 155 | 14.550 | 27.622 | −13.938 | 1.00 | 7.39 |
| ATOM | 1258 | CB | ALA | 155 | 13.530 | 26.520 | −13.807 | 1.00 | 7.77 |
| ATOM | 1259 | C | ALA | 155 | 13.878 | 28.964 | −14.140 | 1.00 | 6.87 |
| ATOM | 1260 | O | ALA | 155 | 13.895 | 29.538 | −15.242 | 1.00 | 7.86 |
| ATOM | 1261 | N | LEU | 156 | 13.323 | 29.547 | −13.083 | 1.00 | 6.99 |
| ATOM | 1262 | CA | LEU | 156 | 12.656 | 30.860 | −13.176 | 1.00 | 5.86 |
| ATOM | 1263 | CB | LEU | 156 | 12.035 | 31.279 | −11.838 | 1.00 | 5.92 |
| ATOM | 1264 | CG | LEU | 156 | 10.864 | 30.434 | −11.365 | 1.00 | 5.99 |
| ATOM | 1265 | CD1 | LEU | 156 | 10.480 | 30.809 | −9.936 | 1.00 | 8.87 |
| ATOM | 1266 | CD2 | LEU | 156 | 9.686 | 30.596 | −12.284 | 1.00 | 11.48 |
| ATOM | 1267 | C | LEU | 156 | 13.640 | 31.933 | −13.642 | 1.00 | 6.11 |
| ATOM | 1268 | O | LEU | 156 | 13.287 | 32.805 | −14.447 | 1.00 | 7.28 |
| ATOM | 1269 | N | LEU | 157 | 14.884 | 31.880 | −13.141 | 1.00 | 6.74 |
| ATOM | 1270 | CA | LEU | 157 | 15.898 | 32.863 | −13.543 | 1.00 | 7.25 |
| ATOM | 1271 | CB | LEU | 157 | 17.152 | 32.739 | −12.694 | 1.00 | 6.79 |
| ATOM | 1272 | CG | LEU | 157 | 17.073 | 33.236 | −11.250 | 1.00 | 7.78 |
| ATOM | 1273 | CD1 | LEU | 157 | 18.408 | 33.026 | −10.580 | 1.00 | 8.46 |
| ATOM | 1274 | CD2 | LEU | 157 | 16.631 | 34.672 | −11.145 | 1.00 | 9.12 |
| ATOM | 1275 | C | LEU | 157 | 16.227 | 32.748 | −15.028 | 1.00 | 6.91 |
| ATOM | 1276 | O | LEU | 157 | 16.580 | 33.763 | −15.638 | 1.00 | 7.24 |
| ATOM | 1277 | N | LYS | 158 | 16.142 | 31.561 | −15.606 | 1.00 | 7.38 |
| ATOM | 1278 | CA | LYS | 158 | 16.335 | 31.410 | −17.057 | 1.00 | 7.07 |
| ATOM | 1279 | CB | LYS | 158 | 16.381 | 29.927 | −17.407 | 1.00 | 9.01 |
| ATOM | 1280 | CG | LYS | 158 | 17.574 | 29.170 | −16.887 | 1.00 | 10.48 |
| ATOM | 1281 | CD | LYS | 158 | 17.541 | 27.683 | −17.122 | 1.00 | 13.42 |
| ATOM | 1282 | CE | LYS | 158 | 18.743 | 27.039 | −16.407 | 1.00 | 18.16 |
| ATOM | 1283 | NZ | LYS | 158 | 18.743 | 25.572 | −16.586 | 1.00 | 18.72 |
| ATOM | 1284 | C | LYS | 158 | 15.185 | 32.121 | −17.782 | 1.00 | 7.00 |
| ATOM | 1285 | O | LYS | 158 | 15.447 | 32.806 | −18.770 | 1.00 | 9.09 |
| ATOM | 1286 | N | GLY | 159 | 13.951 | 32.032 | −17.281 | 1.00 | 7.17 |
| ATOM | 1287 | CA | GLY | 159 | 12.834 | 32.752 | −17.873 | 1.00 | 7.22 |
| ATOM | 1288 | C | GLY | 159 | 13.038 | 34.252 | −17.763 | 1.00 | 6.90 |
| ATOM | 1289 | O | GLY | 159 | 12.756 | 34.973 | −18.731 | 1.00 | 7.59 |
| ATOM | 1290 | N | TYR | 160 | 13.443 | 34.771 | −16.620 | 1.00 | 7.51 |
| ATOM | 1291 | CA | TYR | 160 | 13.674 | 36.230 | −16.489 | 1.00 | 6.84 |
| ATOM | 1292 | CB | TYR | 160 | 14.004 | 36.595 | −15.059 | 1.00 | 7.03 |
| ATOM | 1293 | CG | TYR | 160 | 12.831 | 36.759 | −14.120 | 1.00 | 6.77 |
| ATOM | 1294 | CD1 | TYR | 160 | 12.421 | 35.752 | −13.251 | 1.00 | 6.69 |
| ATOM | 1295 | CE1 | TYR | 160 | 11.353 | 35.915 | −12.430 | 1.00 | 7.61 |
| ATOM | 1296 | CD2 | TYR | 160 | 12.097 | 37.948 | −14.093 | 1.00 | 7.26 |
| ATOM | 1297 | CE2 | TYR | 160 | 11.013 | 38.108 | −13.247 | 1.00 | 7.77 |
| ATOM | 1298 | CZ | TYR | 160 | 10.643 | 37.083 | −12.380 | 1.00 | 7.26 |
| ATOM | 1299 | OH | TYR | 160 | 9.594 | 37.202 | −11.495 | 1.00 | 8.86 |
| ATOM | 1300 | C | TYR | 160 | 14.756 | 36.704 | −17.446 | 1.00 | 6.38 |
| ATOM | 1301 | O | TYR | 160 | 14.653 | 37.777 | −18.003 | 1.00 | 7.25 |
| ATOM | 1302 | N | ALA | 161 | 15.849 | 35.968 | −17.537 | 1.00 | 7.30 |
| ATOM | 1303 | CA | ALA | 161 | 16.929 | 36.375 | −18.438 | 1.00 | 7.63 |
| ATOM | 1304 | CB | ALA | 161 | 18.083 | 35.387 | −18.293 | 1.00 | 9.13 |
| ATOM | 1305 | C | ALA | 161 | 16.414 | 36.456 | −19.886 | 1.00 | 7.60 |
| ATOM | 1306 | O | ALA | 161 | 16.688 | 37.458 | −20.577 | 1.00 | 8.23 |
| ATOM | 1307 | N | LEU | 162 | 15.755 | 35.401 | −20.331 | 1.00 | 7.75 |
| ATOM | 1308 | CA | LEU | 162 | 15.234 | 35.423 | −21.721 | 1.00 | 7.67 |
| ATOM | 1309 | CB | LEU | 162 | 14.537 | 34.094 | −22.022 | 1.00 | 7.94 |
| ATOM | 1310 | CG | LEU | 162 | 15.447 | 32.872 | −22.168 | 1.00 | 8.36 |
| ATOM | 1311 | CD1 | LEU | 162 | 14.644 | 31.593 | −22.325 | 1.00 | 10.23 |
| ATOM | 1312 | CD2 | LEU | 162 | 16.390 | 33.093 | −23.326 | 1.00 | 10.26 |
| ATOM | 1313 | C | LEU | 162 | 14.256 | 36.582 | −21.871 | 1.00 | 7.87 |
| ATOM | 1314 | O | LEU | 162 | 14.228 | 37.234 | −22.926 | 1.00 | 8.10 |
| ATOM | 1315 | N | ALA | 163 | 13.396 | 36.840 | −20.874 | 1.00 | 7.67 |
| ATOM | 1316 | CA | ALA | 163 | 12.422 | 37.934 | −20.922 | 1.00 | 8.06 |
| ATOM | 1317 | CB | ALA | 163 | 11.568 | 37.986 | −19.658 | 1.00 | 8.57 |
| ATOM | 1318 | C | ALA | 163 | 13.122 | 39.278 | −21.162 | 1.00 | 8.14 |
| ATOM | 1319 | O | ALA | 163 | 12.527 | 40.158 | −21.830 | 1.00 | 9.20 |
| ATOM | 1320 | N | LEU | 164 | 14.320 | 39.445 | −20.607 | 1.00 | 8.56 |
| ATOM | 1321 | CA | LEU | 164 | 15.075 | 40.688 | −20.710 | 1.00 | 8.69 |
| ATOM | 1322 | CB | LEU | 164 | 15.881 | 40.917 | −19.409 | 1.00 | 9.34 |
| ATOM | 1323 | CG | LEU | 164 | 15.007 | 41.231 | −18.199 | 1.00 | 9.92 |
| ATOM | 1324 | CD1 | LEU | 164 | 15.707 | 40.869 | −16.893 | 1.00 | 11.23 |
| ATOM | 1325 | CD2 | LEU | 164 | 14.532 | 42.665 | −18.193 | 1.00 | 11.47 |
| ATOM | 1326 | C | LEU | 164 | 15.968 | 40.800 | −21.920 | 1.00 | 8.43 |
| ATOM | 1327 | O | LEU | 164 | 16.728 | 41.751 | −22.068 | 1.00 | 10.53 |
| ATOM | 1328 | N | GLY | 165 | 15.876 | 39.849 | −22.823 | 1.00 | 9.22 |
| ATOM | 1329 | CA | GLY | 165 | 16.653 | 39.841 | −24.034 | 1.00 | 9.48 |
| ATOM | 1330 | C | GLY | 165 | 18.044 | 39.299 | −23.843 | 1.00 | 9.17 |
| ATOM | 1331 | O | GLY | 165 | 18.900 | 39.539 | −24.692 | 1.00 | 10.21 |
| ATOM | 1332 | N | LYS | 166 | 18.298 | 38.579 | −22.754 | 1.00 | 9.48 |
| ATOM | 1333 | CA | LYS | 166 | 19.627 | 38.033 | −22.452 | 1.00 | 9.96 |
| ATOM | 1334 | CB | LYS | 166 | 19.982 | 38.274 | −20.984 | 1.00 | 9.69 |
| ATOM | 1335 | CG | LYS | 166 | 19.946 | 39.708 | −20.549 | 1.00 | 11.03 |
| ATOM | 1336 | CD | LYS | 166 | 20.825 | 40.606 | −21.352 | 1.00 | 14.43 |
| ATOM | 1337 | CE | LYS | 166 | 20.799 | 42.029 | −20.820 | 1.00 | 18.44 |
| ATOM | 1338 | NZ | LYS | 166 | 21.480 | 42.980 | −21.738 | 1.00 | 27.60 |
| ATOM | 1339 | C | LYS | 166 | 19.669 | 36.535 | −22.725 | 1.00 | 9.64 |
| ATOM | 1340 | O | LYS | 166 | 18.611 | 35.926 | −22.985 | 1.00 | 11.70 |
| ATOM | 1341 | N | GLU | 167 | 20.837 | 35.917 | −22.660 | 1.00 | 9.56 |
| ATOM | 1342 | CA | GLU | 167 | 20.979 | 34.447 | −22.758 | 1.00 | 10.09 |
| ATOM | 1343 | CB | GLU | 167 | 22.436 | 34.051 | −22.993 | 1.00 | 12.65 |
| ATOM | 1344 | CG | GLU | 167 | 23.380 | 34.300 | −21.838 | 1.00 | 22.48 |
| ATOM | 1345 | CD | GLU | 167 | 23.665 | 33.149 | −20.891 | 1.00 | 25.40 |
| ATOM | 1346 | OE1 | GLU | 167 | 23.311 | 31.984 | −21.130 | 1.00 | 32.34 |
| ATOM | 1347 | OE2 | GLU | 167 | 24.326 | 33.330 | −19.840 | 1.00 | 31.11 |
| ATOM | 1348 | C | GLU | 167 | 20.484 | 33.838 | −21.438 | 1.00 | 9.49 |
| ATOM | 1349 | O | GLU | 167 | 20.519 | 34.524 | −20.407 | 1.00 | 10.35 |
| ATOM | 1350 | N | GLU | 168 | 19.999 | 32.587 | −21.453 | 1.00 | 9.82 |
| ATOM | 1351 | CA | GLU | 168 | 19.298 | 32.071 | −20.295 | 1.00 | 9.33 |
| ATOM | 1352 | CB | GLU | 168 | 18.638 | 30.725 | −20.586 | 1.00 | 10.98 |
| ATOM | 1353 | CG | GLU | 168 | 19.586 | 29.555 | −20.607 | 1.00 | 11.58 |
| ATOM | 1354 | CD | GLU | 168 | 18.897 | 28.248 | −20.881 | 1.00 | 12.24 |
| ATOM | 1355 | OE1 | GLU | 168 | 19.518 | 27.191 | −20.621 | 1.00 | 16.73 |
| ATOM | 1356 | OE2 | GLU | 168 | 17.710 | 28.209 | −21.238 | 1.00 | 15.75 |
| ATOM | 1357 | C | GLU | 168 | 20.121 | 31.991 | −19.021 | 1.00 | 9.76 |
| ATOM | 1358 | O | GLU | 168 | 19.500 | 31.919 | −17.957 | 1.00 | 10.27 |
| ATOM | 1359 | N | ASN | 169 | 21.440 | 31.959 | −19.098 | 1.00 | 11.16 |
| ATOM | 1360 | CA | ASN | 169 | 22.256 | 31.849 | −17.894 | 1.00 | 11.83 |
| ATOM | 1361 | CB | ASN | 169 | 23.467 | 30.962 | −18.200 | 1.00 | 17.37 |
| ATOM | 1362 | CG | ASN | 169 | 23.037 | 29.534 | −18.442 | 1.00 | 25.58 |
| ATOM | 1363 | OD1 | ASN | 169 | 23.422 | 28.858 | −19.416 | 1.00 | 36.44 |
| ATOM | 1364 | ND2 | ASN | 169 | 22.234 | 29.059 | −17.485 | 1.00 | 33.88 |
| ATOM | 1365 | C | ASN | 169 | 22.665 | 33.191 | −17.342 | 1.00 | 10.67 |
| ATOM | 1366 | O | ASN | 169 | 23.471 | 33.254 | −16.423 | 1.00 | 10.87 |
| ATOM | 1367 | N | PHE | 170 | 22.101 | 34.292 | −17.818 | 1.00 | 9.81 |
| ATOM | 1368 | CA | PHE | 170 | 22.503 | 35.641 | −17.397 | 1.00 | 9.43 |
| ATOM | 1369 | CB | PHE | 170 | 21.676 | 36.690 | −18.110 | 1.00 | 10.54 |
| ATOM | 1370 | CG | PHE | 170 | 21.970 | 38.143 | −17.807 | 1.00 | 10.68 |
| ATOM | 1371 | CD1 | PHE | 170 | 22.971 | 38.748 | −18.561 | 1.00 | 11.73 |
| ATOM | 1372 | CD2 | PHE | 170 | 21.249 | 38.844 | −16.841 | 1.00 | 11.45 |
| ATOM | 1373 | CE1 | PHE | 170 | 23.277 | 40.069 | −18.325 | 1.00 | 13.22 |
| ATOM | 1374 | CE2 | PHE | 170 | 21.601 | 40.156 | −16.603 | 1.00 | 13.30 |
| ATOM | 1375 | CZ | PHE | 170 | 22.581 | 40.787 | −17.369 | 1.00 | 13.73 |
| ATOM | 1376 | C | PHE | 170 | 22.466 | 35.905 | −15.896 | 1.00 | 9.39 |
| ATOM | 1377 | O | PHE | 170 | 23.388 | 36.499 | −15.313 | 1.00 | 10.53 |
| ATOM | 1378 | N | PHE | 171 | 21.414 | 35.405 | −15.249 | 1.00 | 9.21 |
| ATOM | 1379 | CA | PHE | 171 | 21.328 | 35.485 | −13.799 | 1.00 | 9.20 |
| ATOM | 1380 | CB | PHE | 171 | 19.882 | 35.735 | −13.353 | 1.00 | 9.39 |
| ATOM | 1381 | CG | PHE | 171 | 19.270 | 37.023 | −13.840 | 1.00 | 9.53 |
| ATOM | 1382 | CD1 | PHE | 171 | 18.199 | 36.958 | −14.716 | 1.00 | 8.43 |
| ATOM | 1383 | CD2 | PHE | 171 | 19.751 | 38.247 | −13.409 | 1.00 | 9.70 |
| ATOM | 1384 | CE1 | PHE | 171 | 17.637 | 38.140 | −15.183 | 1.00 | 9.69 |
| ATOM | 1385 | CE2 | PHE | 171 | 19.208 | 39.427 | −13.881 | 1.00 | 11.03 |
| ATOM | 1386 | CZ | PHE | 171 | 18.138 | 39.369 | −14.774 | 1.00 | 9.32 |
| ATOM | 1387 | C | PHE | 171 | 21.798 | 34.187 | −13.158 | 1.00 | 9.45 |
| ATOM | 1388 | O | PHE | 171 | 22.557 | 34.196 | −12.160 | 1.00 | 10.11 |
| ATOM | 1389 | N | ALA | 172 | 21.386 | 33.058 | −13.727 | 1.00 | 8.64 |
| ATOM | 1390 | CA | ALA | 172 | 21.662 | 31.758 | −13.141 | 1.00 | 9.25 |
| ATOM | 1391 | CB | ALA | 172 | 20.995 | 30.619 | −13.907 | 1.00 | 11.46 |
| ATOM | 1392 | C | ALA | 172 | 23.144 | 31.456 | −12.935 | 1.00 | 9.80 |
| ATOM | 1393 | O | ALA | 172 | 23.515 | 30.728 | −12.017 | 1.00 | 9.88 |
| ATOM | 1394 | N | ARG | 173 | 24.014 | 32.048 | −13.757 | 1.00 | 10.46 |
| ATOM | 1395 | CA | ARG | 173 | 25.456 | 31.811 | −13.607 | 1.00 | 10.74 |
| ATOM | 1396 | CB | ARG | 173 | 26.229 | 32.377 | −14.804 | 1.00 | 12.02 |
| ATOM | 1397 | CG | ARG | 173 | 26.253 | 33.888 | −14.845 | 1.00 | 14.37 |
| ATOM | 1398 | CD | ARG | 173 | 26.528 | 34.507 | −16.235 | 1.00 | 22.60 |
| ATOM | 1399 | NE | ARG | 173 | 26.118 | 35.904 | −16.110 | 1.00 | 29.23 |
| ATOM | 1400 | CZ | ARG | 173 | 26.204 | 37.054 | −16.666 | 1.00 | 27.71 |
| ATOM | 1401 | NH1 | ARG | 173 | 26.785 | 37.249 | −17.871 | 1.00 | 38.25 |
| ATOM | 1402 | NH2 | ARG | 173 | 25.671 | 38.128 | −16.092 | 1.00 | 20.56 |
| ATOM | 1403 | C | ARG | 173 | 25.967 | 32.398 | −12.321 | 1.00 | 10.00 |
| ATOM | 1404 | O | ARG | 173 | 27.059 | 31.984 | −11.891 | 1.00 | 12.18 |
| ATOM | 1405 | N | HIS | 174 | 25.265 | 33.305 | −11.671 | 1.00 | 8.86 |
| ATOM | 1406 | CA | HIS | 174 | 25.621 | 33.884 | −10.385 | 1.00 | 8.97 |
| ATOM | 1407 | CB | HIS | 174 | 25.281 | 35.380 | −10.393 | 1.00 | 9.34 |
| ATOM | 1408 | CG | HIS | 174 | 25.986 | 36.119 | −11.478 | 1.00 | 11.02 |
| ATOM | 1409 | CD2 | HIS | 174 | 27.271 | 36.548 | −11.356 | 1.00 | 13.05 |
| ATOM | 1410 | ND1 | HIS | 174 | 25.560 | 36.543 | −12.686 | 1.00 | 15.18 |
| ATOM | 1411 | CE1 | HIS | 174 | 26.532 | 37.180 | −13.306 | 1.00 | 16.14 |
| ATOM | 1412 | NE2 | HIS | 174 | 27.575 | 37.238 | −12.505 | 1.00 | 17.60 |
| ATOM | 1413 | C | HIS | 174 | 24.864 | 33.267 | −9.203 | 1.00 | 8.17 |
| ATOM | 1414 | O | HIS | 174 | 25.053 | 33.704 | −8.067 | 1.00 | 10.44 |
| ATOM | 1415 | N | PHE | 175 | 24.093 | 32.251 | −9.482 | 1.00 | 8.57 |
| ATOM | 1416 | CA | PHE | 175 | 23.230 | 31.536 | −8.516 | 1.00 | 7.07 |
| ATOM | 1417 | CB | PHE | 175 | 21.772 | 31.678 | −8.967 | 1.00 | 7.09 |
| ATOM | 1418 | CG | PHE | 175 | 20.743 | 31.096 | −8.020 | 1.00 | 6.00 |
| ATOM | 1419 | CD1 | PHE | 175 | 19.888 | 30.101 | −8.409 | 1.00 | 6.11 |
| ATOM | 1420 | CD2 | PHE | 175 | 20.644 | 31.566 | −6.725 | 1.00 | 6.48 |
| ATOM | 1421 | CE1 | PHE | 175 | 18.940 | 29.615 | −7.548 | 1.00 | 7.35 |
| ATOM | 1422 | CE2 | PHE | 175 | 19.709 | 31.073 | −5.837 | 1.00 | 7.65 |
| ATOM | 1423 | CZ | PHE | 175 | 18.834 | 30.096 | −6.266 | 1.00 | 7.87 |
| ATOM | 1424 | C | PHE | 175 | 23.686 | 30.089 | −8.421 | 1.00 | 7.96 |
| ATOM | 1425 | O | PHE | 175 | 23.298 | 29.250 | −9.248 | 1.00 | 8.40 |
| ATOM | 1426 | N | LYS | 176 | 24.602 | 29.813 | −7.494 | 1.00 | 7.81 |
| ATOM | 1427 | CA | LYS | 176 | 25.320 | 28.576 | −7.473 | 1.00 | 7.94 |
| ATOM | 1428 | CB | LYS | 176 | 26.813 | 28.901 | −7.674 | 1.00 | 10.84 |
| ATOM | 1429 | CG | LYS | 176 | 27.215 | 29.556 | −8.956 | 1.00 | 15.51 |
| ATOM | 1430 | CD | LYS | 176 | 28.584 | 30.190 | −8.877 | 1.00 | 23.52 |
| ATOM | 1431 | CE | LYS | 176 | 28.905 | 31.261 | −7.886 | 1.00 | 31.75 |
| ATOM | 1432 | NZ | LYS | 176 | 28.155 | 32.528 | −7.591 | 1.00 | 30.18 |
| ATOM | 1433 | C | LYS | 176 | 25.206 | 27.890 | −6.114 | 1.00 | 7.44 |
| ATOM | 1434 | O | LYS | 176 | 25.237 | 28.596 | −5.079 | 1.00 | 7.02 |
| ATOM | 1435 | N | PRO | 177 | 25.115 | 26.568 | −6.114 | 1.00 | 7.06 |
| ATOM | 1436 | CD | PRO | 177 | 25.016 | 25.639 | −7.241 | 1.00 | 8.87 |
| ATOM | 1437 | CA | PRO | 177 | 24.978 | 25.874 | −4.841 | 1.00 | 7.22 |
| ATOM | 1438 | CB | PRO | 177 | 24.960 | 24.392 | −5.244 | 1.00 | 9.12 |
| ATOM | 1439 | CG | PRO | 177 | 24.494 | 24.351 | −6.632 | 1.00 | 12.20 |
| ATOM | 1440 | C | PRO | 177 | 26.107 | 26.128 | −3.866 | 1.00 | 6.45 |
| ATOM | 1441 | O | PRO | 177 | 25.842 | 26.133 | −2.655 | 1.00 | 6.96 |
| ATOM | 1442 | N | ASP | 178 | 27.355 | 26.340 | −4.296 | 1.00 | 7.00 |
| ATOM | 1443 | CA | ASP | 178 | 28.432 | 26.494 | −3.322 | 1.00 | 7.82 |
| ATOM | 1444 | CB | ASP | 178 | 29.784 | 26.223 | −3.996 | 1.00 | 7.64 |
| ATOM | 1445 | CG | ASP | 178 | 30.055 | 24.770 | −4.225 | 1.00 | 8.62 |
| ATOM | 1446 | OD1 | ASP | 178 | 29.305 | 23.888 | −3.752 | 1.00 | 10.49 |
| ATOM | 1447 | OD2 | ASP | 178 | 31.072 | 24.504 | −4.927 | 1.00 | 9.66 |
| ATOM | 1448 | C | ASP | 178 | 28.418 | 27.826 | −2.624 | 1.00 | 8.41 |
| ATOM | 1449 | O | ASP | 178 | 29.053 | 27.922 | −1.567 | 1.00 | 11.48 |
| ATOM | 1450 | N | ASP | 179 | 27.747 | 28.844 | −3.172 | 1.00 | 7.61 |
| ATOM | 1451 | CA | ASP | 179 | 27.868 | 30.160 | −2.558 | 1.00 | 8.48 |
| ATOM | 1452 | CB | ASP | 179 | 29.017 | 31.003 | −3.110 | 1.00 | 14.39 |
| ATOM | 1453 | CG | ASP | 179 | 28.896 | 31.215 | −4.582 | 1.00 | 17.08 |
| ATOM | 1454 | OD1 | ASP | 179 | 27.769 | 31.231 | −5.087 | 1.00 | 18.23 |
| ATOM | 1455 | OD2 | ASP | 179 | 29.974 | 31.247 | −5.214 | 1.00 | 29.01 |
| ATOM | 1456 | C | ASP | 179 | 26.622 | 30.999 | −2.437 | 1.00 | 6.67 |
| ATOM | 1457 | O | ASP | 179 | 26.723 | 32.118 | −1.937 | 1.00 | 7.91 |
| ATOM | 1458 | N | THR | 180 | 25.459 | 30.574 | −2.903 | 1.00 | 6.15 |
| ATOM | 1459 | CA | THR | 180 | 24.291 | 31.397 | −2.818 | 1.00 | 5.45 |
| ATOM | 1460 | CB | THR | 180 | 23.061 | 30.717 | −3.495 | 1.00 | 6.14 |
| ATOM | 1461 | OG1 | THR | 180 | 21.933 | 31.564 | −3.275 | 1.00 | 6.60 |
| ATOM | 1462 | CG2 | THR | 180 | 22.728 | 29.384 | −2.872 | 1.00 | 7.19 |
| ATOM | 1463 | C | THR | 180 | 23.929 | 31.823 | −1.404 | 1.00 | 5.42 |
| ATOM | 1464 | O | THR | 180 | 23.995 | 31.022 | −0.488 | 1.00 | 6.17 |
| ATOM | 1465 | N | LEU | 181 | 23.568 | 33.098 | −1.291 | 1.00 | 5.82 |
| ATOM | 1466 | CA | LEU | 181 | 23.100 | 33.709 | −0.086 | 1.00 | 5.33 |
| ATOM | 1467 | CB | LEU | 181 | 23.535 | 35.173 | 0.000 | 1.00 | 5.68 |
| ATOM | 1468 | CG | LEU | 181 | 25.031 | 35.342 | 0.214 | 1.00 | 7.12 |
| ATOM | 1469 | CD1 | LEU | 181 | 25.527 | 36.714 | −0.116 | 1.00 | 9.05 |
| ATOM | 1470 | CD2 | LEU | 181 | 25.431 | 34.952 | 1.631 | 1.00 | 9.04 |
| ATOM | 1471 | C | LEU | 181 | 21.596 | 33.554 | 0.126 | 1.00 | 5.36 |
| ATOM | 1472 | O | LEU | 181 | 21.002 | 34.159 | 1.018 | 1.00 | 5.78 |
| ATOM | 1473 | N | ALA | 182 | 20.943 | 32.746 | −0.705 | 1.00 | 5.62 |
| ATOM | 1474 | CA | ALA | 182 | 19.499 | 32.518 | −0.607 | 1.00 | 5.07 |
| ATOM | 1475 | CB | ALA | 182 | 19.041 | 31.560 | −1.713 | 1.00 | 5.84 |
| ATOM | 1476 | C | ALA | 182 | 19.111 | 31.916 | 0.741 | 1.00 | 5.26 |
| ATOM | 1477 | O | ALA | 182 | 19.829 | 31.152 | 1.390 | 1.00 | 5.11 |
| ATOM | 1478 | N | SER | 183 | 17.929 | 32.266 | 1.215 | 1.00 | 5.45 |
| ATOM | 1479 | CA | SER | 183 | 17.387 | 31.766 | 2.475 | 1.00 | 5.43 |
| ATOM | 1480 | CB | SER | 183 | 17.361 | 32.908 | 3.478 | 1.00 | 6.48 |
| ATOM | 1481 | OG | SER | 183 | 16.484 | 33.920 | 3.050 | 1.00 | 7.39 |
| ATOM | 1482 | C | SER | 183 | 15.975 | 31.229 | 2.287 | 1.00 | 5.28 |
| ATOM | 1483 | O | SER | 183 | 15.220 | 31.691 | 1.430 | 1.00 | 5.14 |
| ATOM | 1484 | N | VAL | 184 | 15.624 | 30.298 | 3.167 | 1.00 | 4.72 |
| ATOM | 1485 | CA | VAL | 184 | 14.272 | 29.833 | 3.379 | 1.00 | 4.18 |
| ATOM | 1486 | CB | VAL | 184 | 14.156 | 28.311 | 3.410 | 1.00 | 4.82 |
| ATOM | 1487 | CG1 | VAL | 184 | 12.784 | 27.827 | 3.825 | 1.00 | 5.78 |
| ATOM | 1488 | CG2 | VAL | 184 | 14.574 | 27.702 | 2.084 | 1.00 | 5.85 |
| ATOM | 1489 | C | VAL | 184 | 13.803 | 30.392 | 4.728 | 1.00 | 4.63 |
| ATOM | 1490 | O | VAL | 184 | 14.563 | 30.341 | 5.712 | 1.00 | 5.58 |
| ATOM | 1491 | N | VAL | 185 | 12.571 | 30.872 | 4.785 | 1.00 | 5.40 |
| ATOM | 1492 | CA | VAL | 185 | 11.960 | 31.260 | 6.049 | 1.00 | 5.11 |
| ATOM | 1493 | CB | VAL | 185 | 11.732 | 32.758 | 6.222 | 1.00 | 5.44 |
| ATOM | 1494 | CG1 | VAL | 185 | 11.355 | 33.084 | 7.659 | 1.00 | 7.04 |
| ATOM | 1495 | CG2 | VAL | 185 | 12.974 | 33.556 | 5.824 | 1.00 | 7.05 |
| ATOM | 1496 | C | VAL | 185 | 10.664 | 30.482 | 6.215 | 1.00 | 5.37 |
| ATOM | 1497 | O | VAL | 185 | 9.793 | 30.537 | 5.354 | 1.00 | 6.79 |
| ATOM | 1498 | N | LEU | 186 | 10.525 | 29.766 | 7.340 | 1.00 | 5.04 |
| ATOM | 1499 | CA | LEU | 186 | 9.312 | 29.030 | 7.659 | 1.00 | 5.37 |
| ATOM | 1500 | CB | LEU | 186 | 9.608 | 27.651 | 8.191 | 1.00 | 6.74 |
| ATOM | 1501 | CG | LEU | 186 | 10.519 | 26.783 | 7.329 | 1.00 | 7.22 |
| ATOM | 1502 | CD1 | LEU | 186 | 10.783 | 25.457 | 8.026 | 1.00 | 8.08 |
| ATOM | 1503 | CD2 | LEU | 186 | 9.933 | 26.589 | 5.923 | 1.00 | 7.11 |
| ATOM | 1504 | C | LEU | 186 | 8.512 | 29.883 | 8.645 | 1.00 | 5.24 |
| ATOM | 1505 | O | LEU | 186 | 8.777 | 29.826 | 9.854 | 1.00 | 6.31 |
| ATOM | 1506 | N | ILE | 187 | 7.577 | 30.701 | 8.165 | 1.00 | 5.56 |
| ATOM | 1507 | CA | ILE | 187 | 6.864 | 31.641 | 9.010 | 1.00 | 5.65 |
| ATOM | 1508 | CB | ILE | 187 | 6.589 | 32.984 | 8.305 | 1.00 | 6.04 |
| ATOM | 1509 | CG2 | ILE | 187 | 5.916 | 33.986 | 9.250 | 1.00 | 7.75 |
| ATOM | 1510 | CG1 | ILE | 187 | 7.804 | 33.601 | 7.633 | 1.00 | 6.80 |
| ATOM | 1511 | CD1 | ILE | 187 | 7.550 | 34.739 | 6.667 | 1.00 | 7.49 |
| ATOM | 1512 | C | ILE | 187 | 5.511 | 31.068 | 9.440 | 1.00 | 6.09 |
| ATOM | 1513 | O | ILE | 187 | 4.740 | 30.565 | 8.617 | 1.00 | 6.78 |
| ATOM | 1514 | N | ARG | 188 | 5.253 | 31.145 | 10.736 | 1.00 | 6.56 |
| ATOM | 1515 | CA | ARG | 188 | 3.970 | 30.800 | 11.332 | 1.00 | 6.28 |
| ATOM | 1516 | CB | ARG | 188 | 4.130 | 29.967 | 12.611 | 1.00 | 7.38 |
| ATOM | 1517 | CG | ARG | 188 | 2.799 | 29.623 | 13.245 | 1.00 | 7.70 |
| ATOM | 1518 | CD | ARG | 188 | 2.926 | 29.038 | 14.641 | 1.00 | 9.29 |
| ATOM | 1519 | NE | ARG | 188 | 1.588 | 28.821 | 15.185 | 1.00 | 11.11 |
| ATOM | 1520 | CZ | ARG | 188 | 1.357 | 28.454 | 16.431 | 1.00 | 12.61 |
| ATOM | 1521 | NH1 | ARG | 188 | 2.357 | 28.232 | 17.264 | 1.00 | 16.10 |
| ATOM | 1522 | NH2 | ARG | 188 | 0.090 | 28.327 | 16.815 | 1.00 | 16.40 |
| ATOM | 1523 | C | ARG | 188 | 3.224 | 32.081 | 11.714 | 1.00 | 6.29 |
| ATOM | 1524 | O | ARG | 188 | 3.714 | 32.892 | 12.499 | 1.00 | 7.23 |
| ATOM | 1525 | N | TYR | 189 | 2.053 | 32.239 | 11.099 | 1.00 | 6.43 |
| ATOM | 1526 | CA | TYR | 189 | 1.129 | 33.301 | 11.534 | 1.00 | 6.36 |
| ATOM | 1527 | CB | TYR | 189 | 0.565 | 33.997 | 10.302 | 1.00 | 7.87 |
| ATOM | 1528 | CG | TYR | 189 | 1.432 | 35.109 | 9.770 | 1.00 | 7.69 |
| ATOM | 1529 | CD1 | TYR | 189 | 2.245 | 34.959 | 8.649 | 1.00 | 8.11 |
| ATOM | 1530 | CE1 | TYR | 189 | 3.023 | 36.028 | 8.202 | 1.00 | 8.55 |
| ATOM | 1531 | CD2 | TYR | 189 | 1.433 | 36.344 | 10.401 | 1.00 | 8.98 |
| ATOM | 1532 | CE2 | TYR | 189 | 2.188 | 37.415 | 9.969 | 1.00 | 9.17 |
| ATOM | 1533 | CZ | TYR | 189 | 2.981 | 37.241 | 8.841 | 1.00 | 8.58 |
| ATOM | 1534 | OH | TYR | 189 | 3.728 | 38.328 | 8.379 | 1.00 | 11.03 |
| ATOM | 1535 | C | TYR | 189 | 0.033 | 32.562 | 12.300 | 1.00 | 7.25 |
| ATOM | 1536 | O | TYR | 189 | −0.678 | 31.766 | 11.696 | 1.00 | 8.05 |
| ATOM | 1537 | N | PRO | 190 | −0.137 | 32.811 | 13.576 | 1.00 | 6.97 |
| ATOM | 1538 | CD | PRO | 190 | 0.666 | 33.760 | 14.412 | 1.00 | 7.48 |
| ATOM | 1539 | CA | PRO | 190 | −1.091 | 32.037 | 14.367 | 1.00 | 7.93 |
| ATOM | 1540 | CB | PRO | 190 | −0.484 | 32.215 | 15.773 | 1.00 | 9.03 |
| ATOM | 1541 | CG | PRO | 190 | −0.032 | 33.667 | 15.732 | 1.00 | 8.65 |
| ATOM | 1542 | C | PRO | 190 | −2.516 | 32.564 | 14.390 | 1.00 | 7.31 |
| ATOM | 1543 | O | PRO | 190 | −2.768 | 33.746 | 14.183 | 1.00 | 7.83 |
| ATOM | 1544 | N | TYR | 191 | −3.428 | 31.672 | 14.740 | 1.00 | 8.07 |
| ATOM | 1545 | CA | TYR | 191 | −4.758 | 32.035 | 15.189 | 1.00 | 8.05 |
| ATOM | 1546 | CB | TYR | 191 | −5.741 | 30.882 | 15.033 | 1.00 | 9.47 |
| ATOM | 1547 | CG | TYR | 191 | −7.089 | 31.164 | 15.645 | 1.00 | 8.74 |
| ATOM | 1548 | CD1 | TYR | 191 | −7.981 | 32.032 | 15.052 | 1.00 | 10.94 |
| ATOM | 1549 | CE1 | TYR | 191 | −9.203 | 32.283 | 15.640 | 1.00 | 12.35 |
| ATOM | 1550 | CD2 | TYR | 191 | −7.434 | 30.628 | 16.867 | 1.00 | 11.98 |
| ATOM | 1551 | CE2 | TYR | 191 | −8.662 | 30.860 | 17.464 | 1.00 | 13.36 |
| ATOM | 1552 | CZ | TYR | 191 | −9.520 | 31.710 | 16.847 | 1.00 | 13.52 |
| ATOM | 1553 | OH | TYR | 191 | −10.758 | 31.949 | 17.411 | 1.00 | 20.40 |
| ATOM | 1554 | C | TYR | 191 | −4.634 | 32.352 | 16.687 | 1.00 | 8.41 |
| ATOM | 1555 | O | TYR | 191 | −4.028 | 31.574 | 17.419 | 1.00 | 10.11 |
| ATOM | 1556 | N | LEU | 192 | −5.188 | 33.493 | 17.089 | 1.00 | 9.02 |
| ATOM | 1557 | CA | LEU | 192 | −5.170 | 33.901 | 18.490 | 1.00 | 9.80 |
| ATOM | 1558 | CB | LEU | 192 | −4.106 | 35.000 | 18.751 | 1.00 | 10.70 |
| ATOM | 1559 | CG | LEU | 192 | −2.670 | 34.624 | 18.449 | 1.00 | 11.38 |
| ATOM | 1560 | CD1 | LEU | 192 | −1.779 | 35.835 | 18.366 | 1.00 | 15.17 |
| ATOM | 1561 | CD2 | LEU | 192 | −2.195 | 33.545 | 19.376 | 1.00 | 14.95 |
| ATOM | 1562 | C | LEU | 192 | −6.524 | 34.466 | 18.870 | 1.00 | 11.59 |
| ATOM | 1563 | O | LEU | 192 | −7.087 | 35.298 | 18.167 | 1.00 | 13.64 |
| ATOM | 1564 | N | ASP | 193 | −7.038 | 34.106 | 20.036 | 1.00 | 14.51 |
| ATOM | 1565 | CA | ASP | 193 | −8.305 | 34.590 | 20.567 | 1.00 | 17.44 |
| ATOM | 1566 | C | ASP | 193 | −8.162 | 34.839 | 22.054 | 1.00 | 18.14 |
| ATOM | 1567 | O | ASP | 193 | −8.094 | 33.884 | 22.833 | 1.00 | 20.69 |
| ATOM | 1568 | CB | ASP | 193 | −9.424 | 33.558 | 20.361 | 1.00 | 19.90 |
| ATOM | 1569 | CG | ASP | 193 | −10.778 | 34.035 | 20.844 | 1.00 | 22.56 |
| ATOM | 1570 | OD1 | ASP | 193 | −10.950 | 35.239 | 21.071 | 1.00 | 31.28 |
| ATOM | 1571 | OD2 | ASP | 193 | −11.705 | 33.195 | 20.904 | 1.00 | 31.66 |
| ATOM | 1572 | N | PRO | 194 | −8.007 | 36.055 | 22.502 | 1.00 | 19.75 |
| ATOM | 1573 | CD | PRO | 194 | −7.751 | 36.353 | 23.938 | 1.00 | 21.02 |
| ATOM | 1574 | CA | PRO | 194 | −8.074 | 37.262 | 21.705 | 1.00 | 20.34 |
| ATOM | 1575 | CB | PRO | 194 | −8.358 | 38.337 | 22.780 | 1.00 | 22.22 |
| ATOM | 1576 | CG | PRO | 194 | −7.623 | 37.846 | 23.976 | 1.00 | 23.24 |
| ATOM | 1577 | C | PRO | 194 | −6.794 | 37.553 | 20.961 | 1.00 | 18.30 |
| ATOM | 1578 | O | PRO | 194 | −5.732 | 37.174 | 21.441 | 1.00 | 20.47 |
| ATOM | 1579 | N | TYR | 195 | −6.908 | 38.261 | 19.844 | 1.00 | 16.09 |
| ATOM | 1580 | CA | TYR | 195 | −5.729 | 38.654 | 19.057 | 1.00 | 13.39 |
| ATOM | 1581 | CB | TYR | 195 | −6.063 | 38.748 | 17.591 | 1.00 | 11.89 |
| ATOM | 1582 | CG | TYR | 195 | −4.857 | 38.589 | 16.678 | 1.00 | 10.40 |
| ATOM | 1583 | CD1 | TYR | 195 | −4.733 | 37.428 | 15.902 | 1.00 | 9.05 |
| ATOM | 1584 | CE1 | TYR | 195 | −3.668 | 37.229 | 15.052 | 1.00 | 9.43 |
| ATOM | 1585 | CD2 | TYR | 195 | −3.867 | 39.556 | 16.532 | 1.00 | 11.25 |
| ATOM | 1586 | CE2 | TYR | 195 | −2.801 | 39.370 | 15.678 | 1.00 | 10.47 |
| ATOM | 1587 | CZ | TYR | 195 | −2.706 | 38.217 | 14.940 | 1.00 | 9.27 |
| ATOM | 1588 | OH | TYR | 195 | −1.631 | 38.037 | 14.084 | 1.00 | 10.15 |
| ATOM | 1589 | C | TYR | 195 | −5.251 | 40.015 | 19.570 | 1.00 | 16.02 |
| ATOM | 1590 | O | TYR | 195 | −6.045 | 40.984 | 19.547 | 1.00 | 19.50 |
| ATOM | 1591 | N | PRO | 196 | −4.015 | 40.101 | 20.038 | 1.00 | 19.81 |
| ATOM | 1592 | CD | PRO | 196 | −2.943 | 39.107 | 19.944 | 1.00 | 20.75 |
| ATOM | 1593 | CA | PRO | 196 | −3.555 | 41.366 | 20.632 | 1.00 | 22.65 |
| ATOM | 1594 | CB | PRO | 196 | −2.113 | 41.061 | 21.050 | 1.00 | 24.78 |
| ATOM | 1595 | CG | PRO | 196 | −1.702 | 39.919 | 20.178 | 1.00 | 25.22 |
| ATOM | 1596 | C | PRO | 196 | −3.528 | 42.534 | 19.659 | 1.00 | 21.80 |
| ATOM | 1597 | O | PRO | 196 | −2.893 | 42.411 | 18.610 | 1.00 | 18.33 |
| ATOM | 1598 | N | ALA | 197 | −4.121 | 43.655 | 20.069 | 1.00 | 20.80 |
| ATOM | 1599 | CA | ALA | 197 | −4.137 | 44.850 | 19.209 | 1.00 | 17.10 |
| ATOM | 1600 | CB | ALA | 197 | −4.992 | 45.932 | 19.849 | 1.00 | 22.74 |
| ATOM | 1601 | C | ALA | 197 | −2.736 | 45.332 | 18.943 | 1.00 | 16.42 |
| ATOM | 1602 | O | ALA | 197 | −2.416 | 45.845 | 17.884 | 1.00 | 14.48 |
| ATOM | 1603 | N | ALA | 198 | −1.806 | 45.093 | 19.872 | 1.00 | 18.94 |
| ATOM | 1604 | CA | ALA | 198 | −0.457 | 45.551 | 19.663 | 1.00 | 18.82 |
| ATOM | 1605 | CB | ALA | 198 | 0.389 | 45.537 | 20.917 | 1.00 | 21.76 |
| ATOM | 1606 | C | ALA | 198 | 0.247 | 44.830 | 18.532 | 1.00 | 20.09 |
| ATOM | 1607 | O | ALA | 198 | 1.209 | 45.380 | 17.989 | 1.00 | 20.87 |
| ATOM | 1608 | N | ALA | 199 | −0.282 | 43.655 | 18.148 | 1.00 | 18.02 |
| ATOM | 1609 | CA | ALA | 199 | 0.284 | 42.883 | 17.055 | 1.00 | 20.04 |
| ATOM | 1610 | CB | ALA | 199 | 0.131 | 41.377 | 17.305 | 1.00 | 22.29 |
| ATOM | 1611 | C | ALA | 199 | −0.405 | 43.201 | 15.745 | 1.00 | 17.65 |
| ATOM | 1612 | O | ALA | 199 | −0.143 | 42.526 | 14.746 | 1.00 | 19.33 |
| ATOM | 1613 | N | ILE | 200 | −1.272 | 44.187 | 15.758 | 1.00 | 14.55 |
| ATOM | 1614 | CA | ILE | 200 | −2.007 | 44.619 | 14.572 | 1.00 | 12.89 |
| ATOM | 1615 | CB | ILE | 200 | −3.524 | 44.490 | 14.735 | 1.00 | 12.11 |
| ATOM | 1616 | CG2 | ILE | 200 | −4.273 | 44.933 | 13.491 | 1.00 | 15.92 |
| ATOM | 1617 | CG1 | ILE | 200 | −3.933 | 43.079 | 15.169 | 1.00 | 12.87 |
| ATOM | 1618 | CD1 | ILE | 200 | −5.369 | 42.887 | 15.559 | 1.00 | 15.12 |
| ATOM | 1619 | C | ILE | 200 | −1.604 | 46.049 | 14.242 | 1.00 | 12.80 |
| ATOM | 1620 | O | ILE | 200 | −1.722 | 46.945 | 15.061 | 1.00 | 15.14 |
| ATOM | 1621 | N | LYS | 201 | −1.079 | 46.217 | 13.030 | 1.00 | 12.46 |
| ATOM | 1622 | CA | LYS | 201 | −0.723 | 47.561 | 12.585 | 1.00 | 13.42 |
| ATOM | 1623 | C | LYS | 201 | −1.842 | 48.108 | 11.711 | 1.00 | 12.82 |
| ATOM | 1624 | O | LYS | 201 | −2.682 | 47.341 | 11.198 | 1.00 | 12.98 |
| ATOM | 1625 | CB | LYS | 201 | 0.575 | 47.555 | 11.793 | 1.00 | 15.55 |
| ATOM | 1626 | CG | LYS | 201 | 1.786 | 47.469 | 12.720 | 1.00 | 22.73 |
| ATOM | 1627 | CD | LYS | 201 | 2.968 | 47.442 | 11.792 | 1.00 | 30.21 |
| ATOM | 1628 | CE | LYS | 201 | 3.330 | 45.969 | 11.633 | 1.00 | 33.55 |
| ATOM | 1629 | NZ | LYS | 201 | 4.352 | 45.602 | 12.674 | 1.00 | 44.14 |
| ATOM | 1630 | N | THR | 202 | −1.844 | 49.414 | 11.525 | 1.00 | 12.30 |
| ATOM | 1631 | CA | THR | 202 | −2.896 | 50.018 | 10.731 | 1.00 | 12.03 |
| ATOM | 1632 | CB | THR | 202 | −3.769 | 50.900 | 11.654 | 1.00 | 14.94 |
| ATOM | 1633 | OG1 | THR | 202 | −4.283 | 50.136 | 12.749 | 1.00 | 20.93 |
| ATOM | 1634 | CG2 | THR | 202 | −4.968 | 51.401 | 10.876 | 1.00 | 16.31 |
| ATOM | 1635 | C | THR | 202 | −2.353 | 50.883 | 9.608 | 1.00 | 10.86 |
| ATOM | 1636 | O | THR | 202 | −1.574 | 51.831 | 9.881 | 1.00 | 12.79 |
| ATOM | 1637 | N | ALA | 203 | −2.710 | 50.593 | 8.362 | 1.00 | 10.61 |
| ATOM | 1638 | CA | ALA | 203 | −2.246 | 51.412 | 7.251 | 1.00 | 10.61 |
| ATOM | 1639 | CB | ALA | 203 | −2.554 | 50.704 | 5.923 | 1.00 | 10.92 |
| ATOM | 1640 | C | ALA | 203 | −2.907 | 52.771 | 7.177 | 1.00 | 11.45 |
| ATOM | 1641 | O | ALA | 203 | −3.927 | 53.003 | 7.798 | 1.00 | 12.75 |
| ATOM | 1642 | N | ALA | 204 | −2.316 | 53.701 | 6.418 | 1.00 | 13.11 |
| ATOM | 1643 | CA | ALA | 204 | −2.922 | 55.016 | 6.262 | 1.00 | 14.24 |
| ATOM | 1644 | CB | ALA | 204 | −2.081 | 55.921 | 5.383 | 1.00 | 17.62 |
| ATOM | 1645 | C | ALA | 204 | −4.312 | 54.951 | 5.666 | 1.00 | 15.18 |
| ATOM | 1646 | O | ALA | 204 | −5.116 | 55.828 | 5.979 | 1.00 | 18.40 |
| ATOM | 1647 | N | ASP | 205 | −4.656 | 53.935 | 4.910 | 1.00 | 14.70 |
| ATOM | 1648 | CA | ASP | 205 | −6.010 | 53.765 | 4.378 | 1.00 | 15.08 |
| ATOM | 1649 | CB | ASP | 205 | −5.939 | 53.130 | 2.979 | 1.00 | 14.53 |
| ATOM | 1650 | CG | ASP | 205 | −5.558 | 51.681 | 2.919 | 1.00 | 13.86 |
| ATOM | 1651 | OD1 | ASP | 205 | −5.431 | 51.036 | 3.978 | 1.00 | 13.58 |
| ATOM | 1652 | OD2 | ASP | 205 | −5.414 | 51.137 | 1.785 | 1.00 | 14.56 |
| ATOM | 1653 | C | ASP | 205 | −6.958 | 53.042 | 5.330 | 1.00 | 13.99 |
| ATOM | 1654 | O | ASP | 205 | −8.100 | 52.729 | 4.944 | 1.00 | 16.91 |
| ATOM | 1655 | N | GLY | 206 | −6.470 | 52.672 | 6.523 | 1.00 | 13.46 |
| ATOM | 1656 | CA | GLY | 206 | −7.305 | 51.999 | 7.498 | 1.00 | 13.34 |
| ATOM | 1657 | C | GLY | 206 | −7.215 | 50.496 | 7.536 | 1.00 | 12.65 |
| ATOM | 1658 | O | GLY | 206 | −7.688 | 49.850 | 8.492 | 1.00 | 15.08 |
| ATOM | 1659 | N | THR | 207 | −6.523 | 49.909 | 6.562 | 1.00 | 11.08 |
| ATOM | 1660 | CA | THR | 207 | −6.383 | 48.461 | 6.501 | 1.00 | 10.33 |
| ATOM | 1661 | CB | THR | 207 | −5.728 | 48.047 | 5.186 | 1.00 | 10.43 |
| ATOM | 1662 | OG1 | THR | 207 | −6.475 | 48.593 | 4.068 | 1.00 | 11.13 |
| ATOM | 1663 | CG2 | THR | 207 | −5.730 | 46.528 | 5.000 | 1.00 | 11.62 |
| ATOM | 1664 | C | THR | 207 | −5.542 | 47.948 | 7.669 | 1.00 | 10.83 |
| ATOM | 1665 | O | THR | 207 | −4.460 | 48.451 | 7.944 | 1.00 | 11.23 |
| ATOM | 1666 | N | LYS | 208 | −6.047 | 46.906 | 8.322 | 1.00 | 10.43 |
| ATOM | 1667 | CA | LYS | 208 | −5.328 | 46.251 | 9.393 | 1.00 | 10.06 |
| ATOM | 1668 | CB | LYS | 208 | −6.299 | 45.472 | 10.281 | 1.00 | 10.90 |
| ATOM | 1669 | CG | LYS | 208 | −7.310 | 46.400 | 10.977 | 1.00 | 15.45 |
| ATOM | 1670 | CD | LYS | 208 | −8.216 | 45.550 | 11.860 | 1.00 | 21.10 |
| ATOM | 1671 | CE | LYS | 208 | −9.070 | 46.277 | 12.858 | 1.00 | 25.92 |
| ATOM | 1672 | NZ | LYS | 208 | −10.228 | 45.455 | 13.309 | 1.00 | 35.34 |
| ATOM | 1673 | C | LYS | 208 | −4.281 | 45.341 | 8.753 | 1.00 | 8.69 |
| ATOM | 1674 | O | LYS | 208 | −4.631 | 44.523 | 7.893 | 1.00 | 8.73 |
| ATOM | 1675 | N | LEU | 209 | −3.058 | 45.470 | 9.211 | 1.00 | 8.66 |
| ATOM | 1676 | CA | LEU | 209 | −1.912 | 44.745 | 8.686 | 1.00 | 8.20 |
| ATOM | 1677 | CB | LEU | 209 | −0.919 | 45.780 | 8.126 | 1.00 | 8.17 |
| ATOM | 1678 | CG | LEU | 209 | −1.407 | 46.777 | 7.084 | 1.00 | 8.96 |
| ATOM | 1679 | CD1 | LEU | 209 | −0.263 | 47.711 | 6.737 | 1.00 | 10.26 |
| ATOM | 1680 | CD2 | LEU | 209 | −1.959 | 46.077 | 5.837 | 1.00 | 9.92 |
| ATOM | 1681 | C | LEU | 209 | −1.149 | 43.969 | 9.733 | 1.00 | 8.39 |
| ATOM | 1682 | O | LEU | 209 | −1.077 | 44.308 | 10.910 | 1.00 | 9.60 |
| ATOM | 1683 | N | SER | 210 | −0.445 | 42.944 | 9.250 | 1.00 | 7.50 |
| ATOM | 1684 | CA | SER | 210 | 0.555 | 42.241 | 10.017 | 1.00 | 8.27 |
| ATOM | 1685 | CB | SER | 210 | 0.481 | 40.717 | 9.879 | 1.00 | 9.13 |
| ATOM | 1686 | OG | SER | 210 | −0.677 | 40.154 | 10.458 | 1.00 | 10.38 |
| ATOM | 1687 | C | SER | 210 | 1.958 | 42.709 | 9.625 | 1.00 | 8.82 |
| ATOM | 1688 | O | SER | 210 | 2.867 | 42.559 | 10.430 | 1.00 | 11.07 |
| ATOM | 1689 | N | PHE | 211 | 2.153 | 43.228 | 8.404 | 1.00 | 8.41 |
| ATOM | 1690 | CA | PHE | 211 | 3.465 | 43.640 | 7.944 | 1.00 | 8.42 |
| ATOM | 1691 | CB | PHE | 211 | 4.294 | 42.502 | 7.351 | 1.00 | 9.04 |
| ATOM | 1692 | CG | PHE | 211 | 5.784 | 42.797 | 7.169 | 1.00 | 8.29 |
| ATOM | 1693 | CD1 | PHE | 211 | 6.656 | 42.844 | 8.237 | 1.00 | 9.90 |
| ATOM | 1694 | CD2 | PHE | 211 | 6.316 | 43.023 | 5.916 | 1.00 | 8.43 |
| ATOM | 1695 | CE1 | PHE | 211 | 8.014 | 43.064 | 8.074 | 1.00 | 9.71 |
| ATOM | 1696 | CE2 | PHE | 211 | 7.649 | 43.256 | 5.735 | 1.00 | 9.43 |
| ATOM | 1697 | CZ | PHE | 211 | 8.515 | 43.240 | 6.804 | 1.00 | 8.86 |
| ATOM | 1698 | C | PHE | 211 | 3.249 | 44.762 | 6.938 | 1.00 | 7.66 |
| ATOM | 1699 | O | PHE | 211 | 2.415 | 44.662 | 6.051 | 1.00 | 8.72 |
| ATOM | 1700 | N | GLU | 212 | 3.963 | 45.856 | 7.143 | 1.00 | 7.99 |
| ATOM | 1701 | CA | GLU | 212 | 3.752 | 47.072 | 6.365 | 1.00 | 7.78 |
| ATOM | 1702 | CB | GLU | 212 | 4.267 | 48.319 | 7.076 | 1.00 | 10.35 |
| ATOM | 1703 | CG | GLU | 212 | 3.201 | 49.142 | 7.809 | 1.00 | 15.96 |
| ATOM | 1704 | CD | GLU | 212 | 2.280 | 49.935 | 6.895 | 1.00 | 17.40 |
| ATOM | 1705 | OE1 | GLU | 212 | 1.606 | 50.866 | 7.415 | 1.00 | 19.49 |
| ATOM | 1706 | OE2 | GLU | 212 | 2.214 | 49.708 | 5.623 | 1.00 | 15.01 |
| ATOM | 1707 | C | GLU | 212 | 4.328 | 46.972 | 4.954 | 1.00 | 7.86 |
| ATOM | 1708 | O | GLU | 212 | 5.006 | 46.040 | 4.555 | 1.00 | 8.28 |
| ATOM | 1709 | N | TRP | 213 | 3.992 | 47.997 | 4.162 | 1.00 | 8.57 |
| ATOM | 1710 | CA | TRP | 213 | 4.464 | 48.141 | 2.806 | 1.00 | 8.31 |
| ATOM | 1711 | CB | TRP | 213 | 3.999 | 49.421 | 2.155 | 1.00 | 8.34 |
| ATOM | 1712 | CG | TRP | 213 | 4.620 | 50.707 | 2.603 | 1.00 | 9.80 |
| ATOM | 1713 | CD2 | TRP | 213 | 5.776 | 51.317 | 2.074 | 1.00 | 11.28 |
| ATOM | 1714 | CE2 | TRP | 213 | 5.977 | 52.520 | 2.784 | 1.00 | 12.37 |
| ATOM | 1715 | CE3 | TRP | 213 | 6.644 | 50.973 | 1.031 | 1.00 | 12.51 |
| ATOM | 1716 | CD1 | TRP | 213 | 4.181 | 51.522 | 3.610 | 1.00 | 10.15 |
| ATOM | 1717 | NE1 | TRP | 213 | 4.980 | 52.619 | 3.734 | 1.00 | 12.18 |
| ATOM | 1718 | CZ2 | TRP | 213 | 7.053 | 53.363 | 2.474 | 1.00 | 14.69 |
| ATOM | 1719 | CZ3 | TRP | 213 | 7.687 | 51.805 | 0.735 | 1.00 | 13.88 |
| ATOM | 1720 | CH2 | TRP | 213 | 7.887 | 52.990 | 1.460 | 1.00 | 15.53 |
| ATOM | 1721 | C | TRP | 213 | 5.991 | 48.089 | 2.741 | 1.00 | 7.51 |
| ATOM | 1722 | O | TRP | 213 | 6.720 | 48.548 | 3.592 | 1.00 | 8.44 |
| ATOM | 1723 | N | HIS | 214 | 6.470 | 47.422 | 1.657 | 1.00 | 7.74 |
| ATOM | 1724 | CA | HIS | 214 | 7.890 | 47.280 | 1.443 | 1.00 | 7.66 |
| ATOM | 1725 | CB | HIS | 214 | 8.495 | 46.246 | 2.447 | 1.00 | 7.45 |
| ATOM | 1726 | CG | HIS | 214 | 7.976 | 44.860 | 2.273 | 1.00 | 7.38 |
| ATOM | 1727 | CD2 | HIS | 214 | 8.527 | 43.765 | 1.682 | 1.00 | 7.28 |
| ATOM | 1728 | ND1 | HIS | 214 | 6.682 | 44.495 | 2.637 | 1.00 | 6.92 |
| ATOM | 1729 | CE1 | HIS | 214 | 6.501 | 43.234 | 2.298 | 1.00 | 7.44 |
| ATOM | 1730 | NE2 | HIS | 214 | 7.581 | 42.759 | 1.734 | 1.00 | 7.83 |
| ATOM | 1731 | C | HIS | 214 | 8.125 | 46.789 | 0.014 | 1.00 | 6.97 |
| ATOM | 1732 | O | HIS | 214 | 7.212 | 46.319 | −0.642 | 1.00 | 7.95 |
| ATOM | 1733 | N | GLU | 215 | 9.384 | 46.910 | −0.408 | 1.00 | 7.78 |
| ATOM | 1734 | CA | GLU | 215 | 9.921 | 46.216 | −1.569 | 1.00 | 8.05 |
| ATOM | 1735 | CB | GLU | 215 | 10.780 | 47.140 | −2.413 | 1.00 | 9.46 |
| ATOM | 1736 | CG | GLU | 215 | 10.056 | 48.276 | −3.051 | 1.00 | 11.54 |
| ATOM | 1737 | CD | GLU | 215 | 10.919 | 49.189 | −3.900 | 1.00 | 16.19 |
| ATOM | 1738 | OE1 | GLU | 215 | 12.160 | 49.042 | −3.934 | 1.00 | 15.97 |
| ATOM | 1739 | OE2 | GLU | 215 | 10.349 | 50.051 | −4.616 | 1.00 | 23.46 |
| ATOM | 1740 | C | GLU | 215 | 10.805 | 45.070 | −1.025 | 1.00 | 8.11 |
| ATOM | 1741 | O | GLU | 215 | 11.385 | 45.184 | 0.071 | 1.00 | 8.95 |
| ATOM | 1742 | N | ASP | 216 | 10.913 | 43.985 | −1.761 | 1.00 | 7.98 |
| ATOM | 1743 | CA | ASP | 216 | 11.723 | 42.873 | −1.305 | 1.00 | 6.84 |
| ATOM | 1744 | CB | ASP | 216 | 11.363 | 41.564 | −1.990 | 1.00 | 7.14 |
| ATOM | 1745 | CG | ASP | 216 | 10.037 | 40.991 | −1.580 | 1.00 | 6.78 |
| ATOM | 1746 | OD1 | ASP | 216 | 9.415 | 41.594 | −0.693 | 1.00 | 7.44 |
| ATOM | 1747 | OD2 | ASP | 216 | 9.648 | 39.909 | −2.098 | 1.00 | 7.77 |
| ATOM | 1748 | C | ASP | 216 | 13.214 | 43.128 | −1.469 | 1.00 | 6.24 |
| ATOM | 1749 | O | ASP | 216 | 13.696 | 43.704 | −2.450 | 1.00 | 7.93 |
| ATOM | 1750 | N | VAL | 217 | 13.931 | 42.555 | −0.509 | 1.00 | 6.61 |
| ATOM | 1751 | CA | VAL | 217 | 15.369 | 42.439 | −0.559 | 1.00 | 6.71 |
| ATOM | 1752 | CB | VAL | 217 | 16.050 | 42.687 | 0.780 | 1.00 | 7.87 |
| ATOM | 1753 | CG1 | VAL | 217 | 17.568 | 42.640 | 0.641 | 1.00 | 9.01 |
| ATOM | 1754 | CG2 | VAL | 217 | 15.613 | 44.001 | 1.353 | 1.00 | 9.65 |
| ATOM | 1755 | C | VAL | 217 | 15.714 | 41.072 | −1.150 | 1.00 | 6.75 |
| ATOM | 1756 | O | VAL | 217 | 15.769 | 40.051 | −0.482 | 1.00 | 7.26 |
| ATOM | 1757 | N | SER | 218 | 15.852 | 41.047 | −2.476 | 1.00 | 6.83 |
| ATOM | 1758 | CA | SER | 218 | 16.056 | 39.850 | −3.257 | 1.00 | 6.43 |
| ATOM | 1759 | CB | SER | 218 | 14.837 | 38.910 | −3.175 | 1.00 | 6.60 |
| ATOM | 1760 | OG | SER | 218 | 13.749 | 39.472 | −3.890 | 1.00 | 6.81 |
| ATOM | 1761 | C | SER | 218 | 16.304 | 40.211 | −4.722 | 1.00 | 5.92 |
| ATOM | 1762 | O | SER | 218 | 16.044 | 41.351 | −5.112 | 1.00 | 6.97 |
| ATOM | 1763 | N | LEU | 219 | 16.723 | 39.209 | −5.488 | 1.00 | 6.04 |
| ATOM | 1764 | CA | LEU | 219 | 16.663 | 39.294 | −6.937 | 1.00 | 6.10 |
| ATOM | 1765 | CB | LEU | 219 | 17.722 | 38.393 | −7.569 | 1.00 | 6.78 |
| ATOM | 1766 | CG | LEU | 219 | 17.728 | 38.321 | −9.097 | 1.00 | 7.82 |
| ATOM | 1767 | CD1 | LEU | 219 | 18.034 | 39.650 | −9.731 | 1.00 | 9.76 |
| ATOM | 1768 | CD2 | LEU | 219 | 18.660 | 37.225 | −9.589 | 1.00 | 8.26 |
| ATOM | 1769 | C | LEU | 219 | 15.218 | 38.982 | −7.341 | 1.00 | 5.92 |
| ATOM | 1770 | O | LEU | 219 | 14.541 | 39.784 | −7.957 | 1.00 | 6.32 |
| ATOM | 1771 | N | ILE | 220 | 14.743 | 37.780 | −6.965 | 1.00 | 5.60 |
| ATOM | 1772 | CA | ILE | 220 | 13.342 | 37.411 | −7.010 | 1.00 | 5.79 |
| ATOM | 1773 | CB | ILE | 220 | 12.950 | 36.552 | −8.228 | 1.00 | 6.51 |
| ATOM | 1774 | CG2 | ILE | 220 | 13.285 | 37.295 | −9.523 | 1.00 | 7.78 |
| ATOM | 1775 | CG1 | ILE | 220 | 13.563 | 35.144 | −8.189 | 1.00 | 6.95 |
| ATOM | 1776 | CD1 | ILE | 220 | 13.002 | 34.222 | −9.250 | 1.00 | 7.96 |
| ATOM | 1777 | C | ILE | 220 | 12.977 | 36.695 | −5.712 | 1.00 | 5.16 |
| ATOM | 1778 | O | ILE | 220 | 13.869 | 36.252 | −4.968 | 1.00 | 5.70 |
| ATOM | 1779 | N | THR | 221 | 11.694 | 36.656 | −5.419 | 1.00 | 5.46 |
| ATOM | 1780 | CA | THR | 221 | 11.121 | 35.981 | −4.263 | 1.00 | 6.07 |
| ATOM | 1781 | CB | THR | 221 | 10.391 | 36.988 | −3.362 | 1.00 | 6.78 |
| ATOM | 1782 | OG1 | THR | 221 | 11.360 | 37.970 | −2.958 | 1.00 | 6.64 |
| ATOM | 1783 | CG2 | THR | 221 | 9.809 | 36.316 | −2.133 | 1.00 | 7.54 |
| ATOM | 1784 | C | THR | 221 | 10.182 | 34.891 | −4.753 | 1.00 | 5.05 |
| ATOM | 1785 | O | THR | 221 | 9.365 | 35.121 | −5.667 | 1.00 | 6.03 |
| ATOM | 1786 | N | VAL | 222 | 10.317 | 33.704 | −4.181 | 1.00 | 5.43 |
| ATOM | 1787 | CA | VAL | 222 | 9.635 | 32.477 | −4.589 | 1.00 | 5.29 |
| ATOM | 1788 | CB | VAL | 222 | 10.610 | 31.467 | −5.188 | 1.00 | 5.74 |
| ATOM | 1789 | CG1 | VAL | 222 | 9.950 | 30.168 | −5.590 | 1.00 | 6.07 |
| ATOM | 1790 | CG2 | VAL | 222 | 11.389 | 32.046 | −6.358 | 1.00 | 6.22 |
| ATOM | 1791 | C | VAL | 222 | 8.867 | 31.924 | −3.379 | 1.00 | 5.62 |
| ATOM | 1792 | O | VAL | 222 | 9.466 | 31.333 | −2.465 | 1.00 | 5.72 |
| ATOM | 1793 | N | LEU | 223 | 7.572 | 32.200 | −3.305 | 1.00 | 5.40 |
| ATOM | 1794 | CA | LEU | 223 | 6.799 | 32.054 | −2.080 | 1.00 | 5.71 |
| ATOM | 1795 | CB | LEU | 223 | 6.211 | 33.454 | −1.749 | 1.00 | 5.73 |
| ATOM | 1796 | CG | LEU | 223 | 5.245 | 33.563 | −0.576 | 1.00 | 5.62 |
| ATOM | 1797 | CD1 | LEU | 223 | 5.992 | 33.305 | 0.748 | 1.00 | 5.34 |
| ATOM | 1798 | CD2 | LEU | 223 | 4.631 | 34.983 | −0.564 | 1.00 | 7.58 |
| ATOM | 1799 | C | LEU | 223 | 5.672 | 31.053 | −2.119 | 1.00 | 5.89 |
| ATOM | 1800 | O | LEU | 223 | 4.853 | 31.106 | −3.012 | 1.00 | 6.54 |
| ATOM | 1801 | N | TYR | 224 | 5.637 | 30.185 | −1.112 | 1.00 | 5.35 |
| ATOM | 1802 | CA | TYR | 224 | 4.493 | 29.307 | −0.858 | 1.00 | 5.93 |
| ATOM | 1803 | CB | TYR | 224 | 4.891 | 27.865 | −0.543 | 1.00 | 7.37 |
| ATOM | 1804 | CG | TYR | 224 | 3.696 | 27.082 | −0.030 | 1.00 | 8.60 |
| ATOM | 1805 | CD1 | TYR | 224 | 2.805 | 26.474 | −0.888 | 1.00 | 10.86 |
| ATOM | 1806 | CE1 | TYR | 224 | 1.698 | 25.762 | −0.350 | 1.00 | 11.92 |
| ATOM | 1807 | CD2 | TYR | 224 | 3.459 | 26.927 | 1.341 | 1.00 | 10.88 |
| ATOM | 1808 | CE2 | TYR | 224 | 2.363 | 26.334 | 1.897 | 1.00 | 12.37 |
| ATOM | 1809 | CZ | TYR | 224 | 1.500 | 25.717 | 1.012 | 1.00 | 12.52 |
| ATOM | 1810 | OH | TYR | 224 | 0.388 | 25.081 | 1.575 | 1.00 | 16.93 |
| ATOM | 1811 | C | TYR | 224 | 3.702 | 29.956 | 0.284 | 1.00 | 5.19 |
| ATOM | 1812 | O | TYR | 224 | 4.318 | 30.259 | 1.319 | 1.00 | 6.49 |
| ATOM | 1813 | N | GLN | 225 | 2.391 | 29.993 | 0.209 | 1.00 | 5.70 |
| ATOM | 1814 | CA | GLN | 225 | 1.552 | 30.327 | 1.336 | 1.00 | 6.51 |
| ATOM | 1815 | CB | GLN | 225 | 1.053 | 31.753 | 1.362 | 1.00 | 8.15 |
| ATOM | 1816 | CG | GLN | 225 | 2.113 | 32.840 | 1.196 | 1.00 | 8.18 |
| ATOM | 1817 | CD | GLN | 225 | 1.591 | 34.205 | 1.582 | 1.00 | 8.60 |
| ATOM | 1818 | OE1 | GLN | 225 | 2.147 | 34.911 | 2.439 | 1.00 | 11.27 |
| ATOM | 1819 | NE2 | GLN | 225 | 0.536 | 34.580 | 0.927 | 1.00 | 9.01 |
| ATOM | 1820 | C | GLN | 225 | 0.332 | 29.411 | 1.366 | 1.00 | 7.75 |
| ATOM | 1821 | O | GLN | 225 | −0.163 | 28.937 | 0.352 | 1.00 | 8.52 |
| ATOM | 1822 | N | SER | 226 | −0.198 | 29.227 | 2.575 | 1.00 | 9.34 |
| ATOM | 1823 | CA | SER | 226 | −1.464 | 28.544 | 2.756 | 1.00 | 10.96 |
| ATOM | 1824 | CB | SER | 226 | −2.003 | 28.665 | 4.164 | 1.00 | 16.30 |
| ATOM | 1825 | OG | SER | 226 | −1.115 | 28.419 | 5.120 | 1.00 | 15.06 |
| ATOM | 1826 | C | SER | 226 | −2.559 | 29.310 | 2.014 | 1.00 | 10.35 |
| ATOM | 1827 | O | SER | 226 | −2.481 | 30.484 | 1.699 | 1.00 | 11.80 |
| ATOM | 1828 | N | ASN | 227 | −3.684 | 28.605 | 1.934 | 1.00 | 10.66 |
| ATOM | 1829 | CA | ASN | 227 | −4.840 | 29.105 | 1.234 | 1.00 | 11.50 |
| ATOM | 1830 | CB | ASN | 227 | −5.725 | 27.953 | 0.769 | 1.00 | 15.38 |
| ATOM | 1831 | CG | ASN | 227 | −6.303 | 28.312 | −0.576 | 1.00 | 22.10 |
| ATOM | 1832 | OD1 | ASN | 227 | −6.033 | 27.643 | −1.583 | 1.00 | 35.45 |
| ATOM | 1833 | ND2 | ASN | 227 | −6.869 | 29.471 | −0.713 | 1.00 | 20.67 |
| ATOM | 1834 | C | ASN | 227 | −5.668 | 30.072 | 2.070 | 1.00 | 12.61 |
| ATOM | 1835 | O | ASN | 227 | −6.857 | 29.812 | 2.296 | 1.00 | 15.01 |
| ATOM | 1836 | N | VAL | 228 | −5.078 | 31.162 | 2.514 | 1.00 | 10.77 |
| ATOM | 1837 | CA | VAL | 228 | −5.746 | 32.223 | 3.268 | 1.00 | 9.90 |
| ATOM | 1838 | CB | VAL | 228 | −5.417 | 32.224 | 4.768 | 1.00 | 11.72 |
| ATOM | 1839 | CG1 | VAL | 228 | −6.173 | 33.357 | 5.454 | 1.00 | 14.80 |
| ATOM | 1840 | CG2 | VAL | 228 | −5.721 | 30.872 | 5.403 | 1.00 | 14.80 |
| ATOM | 1841 | C | VAL | 228 | −5.284 | 33.531 | 2.644 | 1.00 | 9.82 |
| ATOM | 1842 | O | VAL | 228 | −4.093 | 33.821 | 2.657 | 1.00 | 10.96 |
| ATOM | 1843 | N | GLN | 229 | −6.185 | 34.288 | 2.031 | 1.00 | 9.94 |
| ATOM | 1844 | CA | GLN | 229 | −5.815 | 35.512 | 1.345 | 1.00 | 9.11 |
| ATOM | 1845 | CB | GLN | 229 | −7.038 | 36.050 | 0.595 | 1.00 | 10.74 |
| ATOM | 1846 | CG | GLN | 229 | −6.750 | 37.084 | −0.481 | 1.00 | 10.34 |
| ATOM | 1847 | CD | GLN | 229 | −6.454 | 38.479 | 0.038 | 1.00 | 11.49 |
| ATOM | 1848 | OE1 | GLN | 229 | −7.057 | 38.953 | 1.011 | 1.00 | 12.74 |
| ATOM | 1849 | NE2 | GLN | 229 | −5.440 | 39.124 | −0.556 | 1.00 | 10.77 |
| ATOM | 1850 | C | GLN | 229 | −5.228 | 36.489 | 2.340 | 1.00 | 9.22 |
| ATOM | 1851 | O | GLN | 229 | −5.784 | 36.712 | 3.421 | 1.00 | 10.98 |
| ATOM | 1852 | N | ASN | 230 | −4.133 | 37.140 | 1.924 | 1.00 | 8.54 |
| ATOM | 1853 | CA | ASN | 230 | −3.504 | 38.106 | 2.839 | 1.00 | 9.75 |
| ATOM | 1854 | CB | ASN | 230 | −2.642 | 37.356 | 3.865 | 1.00 | 10.52 |
| ATOM | 1855 | CG | ASN | 230 | −1.468 | 36.649 | 3.229 | 1.00 | 11.42 |
| ATOM | 1856 | OD1 | ASN | 230 | −1.601 | 35.602 | 2.559 | 1.00 | 12.95 |
| ATOM | 1857 | ND2 | ASN | 230 | −0.321 | 37.258 | 3.367 | 1.00 | 10.36 |
| ATOM | 1858 | C | ASN | 230 | −2.684 | 39.177 | 2.186 | 1.00 | 9.22 |
| ATOM | 1859 | O | ASN | 230 | −2.579 | 40.278 | 2.699 | 1.00 | 10.88 |
| ATOM | 1860 | N | LEU | 231 | −2.080 | 38.928 | 1.008 | 1.00 | 8.35 |
| ATOM | 1861 | CA | LEU | 231 | −1.187 | 39.891 | 0.391 | 1.00 | 7.65 |
| ATOM | 1862 | CB | LEU | 231 | −0.166 | 39.130 | −0.486 | 1.00 | 9.10 |
| ATOM | 1863 | CG | LEU | 231 | 0.859 | 38.294 | 0.253 | 1.00 | 10.43 |
| ATOM | 1864 | CD1 | LEU | 231 | 1.718 | 37.494 | −0.719 | 1.00 | 12.85 |
| ATOM | 1865 | CD2 | LEU | 231 | 1.707 | 39.142 | 1.167 | 1.00 | 18.74 |
| ATOM | 1866 | C | LEU | 231 | −1.921 | 40.933 | −0.444 | 1.00 | 7.60 |
| ATOM | 1867 | O | LEU | 231 | −2.902 | 40.618 | −1.110 | 1.00 | 8.69 |
| ATOM | 1868 | N | GLN | 232 | −1.378 | 42.167 | −0.446 | 1.00 | 7.25 |
| ATOM | 1869 | CA | GLN | 232 | −1.884 | 43.212 | −1.298 | 1.00 | 7.79 |
| ATOM | 1870 | CB | GLN | 232 | −2.611 | 44.274 | −0.523 | 1.00 | 8.77 |
| ATOM | 1871 | CG | GLN | 232 | −3.852 | 43.777 | 0.218 | 1.00 | 8.38 |
| ATOM | 1872 | CD | GLN | 232 | −4.616 | 44.915 | 0.825 | 1.00 | 10.24 |
| ATOM | 1873 | OE1 | GLN | 232 | −4.059 | 45.793 | 1.487 | 1.00 | 11.66 |
| ATOM | 1874 | NE2 | GLN | 232 | −5.935 | 44.904 | 0.648 | 1.00 | 14.46 |
| ATOM | 1875 | C | GLN | 232 | −0.682 | 43.819 | −2.003 | 1.00 | 6.81 |
| ATOM | 1876 | O | GLN | 232 | 0.375 | 43.965 | −1.390 | 1.00 | 7.95 |
| ATOM | 1877 | N | VAL | 233 | −0.905 | 44.246 | −3.244 | 1.00 | 7.92 |
| ATOM | 1878 | CA | VAL | 233 | 0.087 | 44.893 | −4.061 | 1.00 | 8.46 |
| ATOM | 1879 | CB | VAL | 233 | 0.462 | 44.130 | −5.339 | 1.00 | 9.34 |
| ATOM | 1880 | CG1 | VAL | 233 | −0.719 | 43.775 | −6.224 | 1.00 | 10.46 |
| ATOM | 1881 | CG2 | VAL | 233 | 1.534 | 44.837 | −6.141 | 1.00 | 9.44 |
| ATOM | 1882 | C | VAL | 233 | −0.381 | 46.307 | −4.399 | 1.00 | 8.77 |
| ATOM | 1883 | O | VAL | 233 | −1.556 | 46.486 | −4.737 | 1.00 | 10.72 |
| ATOM | 1884 | N | GLU | 234 | 0.489 | 47.302 | −4.314 | 1.00 | 8.62 |
| ATOM | 1885 | CA | GLU | 234 | 0.169 | 48.654 | −4.763 | 1.00 | 10.41 |
| ATOM | 1886 | CB | GLU | 234 | 1.095 | 49.693 | −4.115 | 1.00 | 11.08 |
| ATOM | 1887 | CG | GLU | 234 | 0.638 | 51.126 | −4.319 | 1.00 | 12.76 |
| ATOM | 1888 | CD | GLU | 234 | 1.488 | 52.093 | −3.531 | 1.00 | 13.73 |
| ATOM | 1889 | OE1 | GLU | 234 | 2.730 | 51.975 | −3.567 | 1.00 | 16.36 |
| ATOM | 1890 | OE2 | GLU | 234 | 0.903 | 53.025 | −2.925 | 1.00 | 16.64 |
| ATOM | 1891 | C | GLU | 234 | 0.277 | 48.736 | −6.285 | 1.00 | 12.14 |
| ATOM | 1892 | O | GLU | 234 | 1.295 | 48.366 | −6.885 | 1.00 | 13.51 |
| ATOM | 1893 | N | THR | 235 | −0.753 | 49.285 | −6.917 | 1.00 | 15.59 |
| ATOM | 1894 | CA | THR | 235 | −0.699 | 49.507 | −8.364 | 1.00 | 17.29 |
| ATOM | 1895 | CB | THR | 235 | −1.595 | 48.591 | −9.200 | 1.00 | 19.13 |
| ATOM | 1896 | OG1 | THR | 235 | −2.984 | 48.842 | −8.938 | 1.00 | 21.58 |
| ATOM | 1897 | OG2 | THR | 235 | −1.375 | 47.100 | −8.952 | 1.00 | 22.36 |
| ATOM | 1898 | C | THR | 235 | −1.137 | 50.959 | −8.576 | 1.00 | 18.85 |
| ATOM | 1899 | O | THR | 235 | −1.394 | 51.656 | −7.579 | 1.00 | 19.19 |
| ATOM | 1900 | N | ALA | 236 | −1.293 | 51.330 | −9.856 | 1.00 | 22.89 |
| ATOM | 1901 | CA | ALA | 236 | −1.784 | 52.664 | −10.222 | 1.00 | 23.88 |
| ATOM | 1902 | CB | ALA | 236 | −1.749 | 52.884 | −11.725 | 1.00 | 31.13 |
| ATOM | 1903 | C | ALA | 236 | −3.206 | 52.889 | −9.731 | 1.00 | 24.49 |
| ATOM | 1904 | O | ALA | 236 | −3.723 | 53.962 | −9.427 | 1.00 | 31.36 |
| ATOM | 1905 | N | ALA | 237 | −3.929 | 51.774 | −9.619 | 1.00 | 24.56 |
| ATOM | 1906 | CA | ALA | 237 | −5.299 | 51.889 | −9.150 | 1.00 | 25.87 |
| ATOM | 1907 | CB | ALA | 237 | −6.155 | 50.804 | −9.815 | 1.00 | 33.93 |
| ATOM | 1908 | C | ALA | 237 | −5.332 | 51.715 | −7.648 | 1.00 | 24.45 |
| ATOM | 1909 | O | ALA | 237 | −6.424 | 51.484 | −7.120 | 1.00 | 30.47 |
| ATOM | 1910 | N | GLY | 238 | −4.223 | 51.758 | −6.932 | 1.00 | 21.71 |
| ATOM | 1911 | CA | GLY | 238 | −4.269 | 51.563 | −5.481 | 1.00 | 19.83 |
| ATOM | 1912 | C | GLY | 238 | −3.842 | 50.185 | −5.013 | 1.00 | 17.46 |
| ATOM | 1913 | O | GLY | 238 | −3.387 | 49.372 | −5.837 | 1.00 | 17.96 |
| ATOM | 1914 | N | TYR | 239 | −4.061 | 49.844 | −3.728 | 1.00 | 15.86 |
| ATOM | 1915 | CA | TYR | 239 | −3.721 | 48.501 | −3.275 | 1.00 | 12.97 |
| ATOM | 1916 | CB | TYR | 239 | −3.579 | 48.458 | −1.743 | 1.00 | 12.30 |
| ATOM | 1917 | CG | TYR | 239 | −2.235 | 48.936 | −1.252 | 1.00 | 11.99 |
| ATOM | 1918 | CD1 | TYR | 239 | −2.034 | 50.267 | −0.939 | 1.00 | 12.25 |
| ATOM | 1919 | CE1 | TYR | 239 | −0.787 | 50.701 | −0.501 | 1.00 | 12.55 |
| ATOM | 1920 | CD2 | TYR | 239 | −1.161 | 48.056 | −1.168 | 1.00 | 11.54 |
| ATOM | 1921 | CE2 | TYR | 239 | 0.072 | 48.470 | −0.746 | 1.00 | 11.38 |
| ATOM | 1922 | CZ | TYR | 239 | 0.238 | 49.811 | −0.426 | 1.00 | 11.23 |
| ATOM | 1923 | OH | TYR | 239 | 1.484 | 50.235 | −0.015 | 1.00 | 14.40 |
| ATOM | 1924 | C | TYR | 239 | −4.815 | 47.516 | −3.705 | 1.00 | 12.53 |
| ATOM | 1925 | O | TYR | 239 | −6.007 | 47.800 | −3.513 | 1.00 | 17.24 |
| ATOM | 1926 | N | GLN | 240 | −4.409 | 46.398 | −4.296 | 1.00 | 11.52 |
| ATOM | 1927 | CA | GLN | 240 | −5.297 | 45.349 | −4.769 | 1.00 | 11.73 |
| ATOM | 1928 | CB | GLN | 240 | −5.117 | 45.199 | −6.306 | 1.00 | 11.64 |
| ATOM | 1929 | CG | GLN | 240 | −5.539 | 46.485 | −7.051 | 1.00 | 14.46 |
| ATOM | 1930 | CD | GLN | 240 | −5.418 | 46.379 | −8.546 | 1.00 | 16.63 |
| ATOM | 1931 | OE1 | GLN | 240 | −4.585 | 45.635 | −9.069 | 1.00 | 18.79 |
| ATOM | 1932 | NE2 | GLN | 240 | −6.217 | 47.136 | −9.296 | 1.00 | 21.51 |
| ATOM | 1933 | C | GLN | 240 | −4.935 | 44.026 | −4.113 | 1.00 | 10.88 |
| ATOM | 1934 | O | GLN | 240 | −3.763 | 43.770 | −3.789 | 1.00 | 9.92 |
| ATOM | 1935 | N | ASP | 241 | −5.922 | 43.182 | −3.859 | 1.00 | 10.67 |
| ATOM | 1936 | CA | ASP | 241 | −5.711 | 41.915 | −3.230 | 1.00 | 9.82 |
| ATOM | 1937 | CB | ASP | 241 | −7.072 | 41.409 | −2.727 | 1.00 | 11.01 |
| ATOM | 1938 | CG | ASP | 241 | −7.532 | 41.987 | −1.420 | 1.00 | 12.59 |
| ATOM | 1939 | OD1 | ASP | 241 | −8.763 | 41.911 | −1.141 | 1.00 | 15.81 |
| ATOM | 1940 | OD2 | ASP | 241 | −6.719 | 42.478 | −0.648 | 1.00 | 12.84 |
| ATOM | 1941 | C | ASP | 241 | −5.124 | 40.876 | −4.183 | 1.00 | 10.03 |
| ATOM | 1942 | O | ASP | 241 | −5.613 | 40.615 | −5.276 | 1.00 | 13.99 |
| ATOM | 1943 | N | ILE | 242 | −4.082 | 40.170 | −3.742 | 1.00 | 8.45 |
| ATOM | 1944 | CA | ILE | 242 | −3.524 | 39.035 | −4.454 | 1.00 | 8.44 |
| ATOM | 1945 | CB | ILE | 242 | −2.024 | 38.896 | −4.197 | 1.00 | 8.68 |
| ATOM | 1946 | CG2 | ILE | 242 | −1.499 | 37.564 | −4.760 | 1.00 | 10.88 |
| ATOM | 1947 | CG1 | ILE | 242 | −1.271 | 40.096 | −4.766 | 1.00 | 9.70 |
| ATOM | 1948 | CD1 | ILE | 242 | 0.191 | 40.223 | −4.344 | 1.00 | 11.78 |
| ATOM | 1949 | C | ILE | 242 | −4.242 | 37.784 | −3.942 | 1.00 | 8.96 |
| ATOM | 1950 | O | ILE | 242 | −4.216 | 37.474 | −2.740 | 1.00 | 9.65 |
| ATOM | 1951 | N | ALA | 243 | −4.926 | 37.054 | −4.821 | 1.00 | 9.75 |
| ATOM | 1952 | CA | ALA | 243 | −5.595 | 35.814 | −4.414 | 1.00 | 9.73 |
| ATOM | 1953 | C | ALA | 243 | −4.574 | 34.776 | −3.950 | 1.00 | 8.94 |
| ATOM | 1954 | O | ALA | 243 | −3.463 | 34.685 | −4.478 | 1.00 | 11.10 |
| ATOM | 1955 | CB | ALA | 243 | −6.379 | 35.243 | −5.605 | 1.00 | 14.74 |
| ATOM | 1956 | N | ALA | 244 | −4.991 | 33.948 | −2.985 | 1.00 | 9.36 |
| ATOM | 1957 | CA | ALA | 244 | −4.136 | 32.844 | −2.547 | 1.00 | 9.36 |
| ATOM | 1958 | CB | ALA | 244 | −4.588 | 32.409 | −1.156 | 1.00 | 13.93 |
| ATOM | 1959 | C | ALA | 244 | −4.243 | 31.707 | −3.540 | 1.00 | 10.49 |
| ATOM | 1960 | O | ALA | 244 | −5.263 | 31.547 | −4.224 | 1.00 | 13.13 |
| ATOM | 1961 | N | ASP | 245 | −3.215 | 30.899 | −3.615 | 1.00 | 10.92 |
| ATOM | 1962 | CA | ASP | 245 | −3.158 | 29.661 | −4.408 | 1.00 | 11.37 |
| ATOM | 1963 | CB | ASP | 245 | −2.804 | 29.876 | −5.858 | 1.00 | 11.89 |
| ATOM | 1964 | CG | ASP | 245 | −2.892 | 28.664 | −6.747 | 1.00 | 13.54 |
| ATOM | 1965 | OD1 | ASP | 245 | −3.031 | 28.761 | −7.996 | 1.00 | 18.77 |
| ATOM | 1966 | OD2 | ASP | 245 | −2.886 | 27.549 | −6.197 | 1.00 | 13.74 |
| ATOM | 1967 | C | ASP | 245 | −2.109 | 28.779 | −3.720 | 1.00 | 10.46 |
| ATOM | 1968 | O | ASP | 245 | −0.917 | 28.912 | −3.998 | 1.00 | 11.30 |
| ATOM | 1969 | N | ASP | 246 | −2.562 | 27.850 | −2.905 | 1.00 | 9.06 |
| ATOM | 1970 | CA | ASP | 246 | −1.677 | 26.921 | −2.197 | 1.00 | 9.74 |
| ATOM | 1971 | CB | ASP | 246 | −2.253 | 26.499 | −0.859 | 1.00 | 11.33 |
| ATOM | 1972 | CG | ASP | 246 | −3.378 | 25.495 | −1.011 | 1.00 | 13.92 |
| ATOM | 1973 | OD1 | ASP | 246 | −3.834 | 24.958 | 0.018 | 1.00 | 15.40 |
| ATOM | 1974 | OD2 | ASP | 246 | −3.870 | 25.292 | −2.155 | 1.00 | 14.85 |
| ATOM | 1975 | C | ASP | 246 | −1.201 | 25.755 | −3.060 | 1.00 | 9.71 |
| ATOM | 1976 | O | ASP | 246 | −0.571 | 24.838 | −2.544 | 1.00 | 10.59 |
| ATOM | 1977 | N | THR | 247 | −1.417 | 25.840 | −4.379 | 1.00 | 9.46 |
| ATOM | 1978 | CA | THR | 247 | −0.834 | 24.865 | −5.261 | 1.00 | 9.37 |
| ATOM | 1979 | CB | THR | 247 | −1.845 | 24.275 | −6.269 | 1.00 | 11.29 |
| ATOM | 1980 | OG1 | THR | 247 | −2.235 | 25.290 | −7.212 | 1.00 | 11.39 |
| ATOM | 1981 | CG2 | THR | 247 | −3.094 | 23.800 | −5.524 | 1.00 | 12.30 |
| ATOM | 1982 | C | THR | 247 | 0.328 | 25.442 | −6.058 | 1.00 | 8.75 |
| ATOM | 1983 | O | THR | 247 | 1.026 | 24.639 | −6.698 | 1.00 | 11.70 |
| ATOM | 1984 | N | GLY | 248 | 0.477 | 26.744 | −6.088 | 1.00 | 8.16 |
| ATOM | 1985 | CA | GLY | 248 | 1.475 | 27.454 | −6.872 | 1.00 | 8.63 |
| ATOM | 1986 | C | GLY | 248 | 2.494 | 28.172 | −5.992 | 1.00 | 7.49 |
| ATOM | 1987 | O | GLY | 248 | 2.373 | 28.246 | −4.757 | 1.00 | 10.66 |
| ATOM | 1988 | N | TYR | 249 | 3.496 | 28.733 | −6.663 | 1.00 | 6.67 |
| ATOM | 1989 | CA | TYR | 249 | 4.437 | 29.616 | −6.005 | 1.00 | 6.51 |
| ATOM | 1990 | CB | TYR | 249 | 5.900 | 29.244 | −6.303 | 1.00 | 6.70 |
| ATOM | 1991 | CG | TYR | 249 | 6.411 | 28.129 | −5.426 | 1.00 | 6.10 |
| ATOM | 1992 | CD1 | TYR | 249 | 6.314 | 26.788 | −5.752 | 1.00 | 6.62 |
| ATOM | 1993 | CE1 | TYR | 249 | 6.786 | 25.803 | −4.929 | 1.00 | 7.14 |
| ATOM | 1994 | CD2 | TYR | 249 | 6.992 | 28.437 | −4.199 | 1.00 | 5.64 |
| ATOM | 1995 | CE2 | TYR | 249 | 7.470 | 27.450 | −3.364 | 1.00 | 5.23 |
| ATOM | 1996 | CZ | TYR | 249 | 7.367 | 26.135 | −3.715 | 1.00 | 6.30 |
| ATOM | 1997 | OH | TYR | 249 | 7.823 | 25.130 | −2.903 | 1.00 | 8.55 |
| ATOM | 1998 | C | TYR | 249 | 4.202 | 31.046 | −6.523 | 1.00 | 5.67 |
| ATOM | 1999 | O | TYR | 249 | 4.146 | 31.252 | −7.740 | 1.00 | 6.93 |
| ATOM | 2000 | N | LEU | 250 | 4.045 | 31.976 | −5.592 | 1.00 | 5.56 |
| ATOM | 2001 | CA | LEU | 250 | 3.894 | 33.381 | −5.926 | 1.00 | 5.35 |
| ATOM | 2002 | CB | LEU | 250 | 3.151 | 34.119 | −4.803 | 1.00 | 6.61 |
| ATOM | 2003 | CG | LEU | 250 | 2.830 | 35.587 | −5.134 | 1.00 | 6.78 |
| ATOM | 2004 | CD1 | LEU | 250 | 1.810 | 35.727 | −6.251 | 1.00 | 7.40 |
| ATOM | 2005 | CD2 | LEU | 250 | 2.316 | 36.240 | −3.857 | 1.00 | 8.54 |
| ATOM | 2006 | C | LEU | 250 | 5.283 | 33.945 | −6.147 | 1.00 | 5.86 |
| ATOM | 2007 | O | LEU | 250 | 6.157 | 33.797 | −5.272 | 1.00 | 5.91 |
| ATOM | 2008 | N | ILE | 251 | 5.498 | 34.540 | −7.312 | 1.00 | 5.45 |
| ATOM | 2009 | CA | ILE | 251 | 6.777 | 35.088 | −7.727 | 1.00 | 6.03 |
| ATOM | 2010 | CB | ILE | 251 | 7.240 | 34.434 | −9.059 | 1.00 | 6.08 |
| ATOM | 2011 | CG2 | ILE | 251 | 8.728 | 34.801 | −9.306 | 1.00 | 7.80 |
| ATOM | 2012 | CG1 | ILE | 251 | 6.975 | 32.941 | −9.095 | 1.00 | 5.58 |
| ATOM | 2013 | CD1 | ILE | 251 | 7.657 | 32.110 | −8.038 | 1.00 | 6.65 |
| ATOM | 2014 | C | ILE | 251 | 6.712 | 36.599 | −7.909 | 1.00 | 6.10 |
| ATOM | 2015 | O | ILE | 251 | 5.735 | 37.111 | −8.484 | 1.00 | 6.96 |
| ATOM | 2016 | N | ASN | 252 | 7.760 | 37.291 | −7.443 | 1.00 | 6.11 |
| ATOM | 2017 | CA | ASN | 252 | 7.892 | 38.703 | −7.696 | 1.00 | 6.61 |
| ATOM | 2018 | CB | ASN | 252 | 7.145 | 39.615 | −6.739 | 1.00 | 7.62 |
| ATOM | 2019 | CG | ASN | 252 | 7.617 | 39.521 | −5.306 | 1.00 | 8.27 |
| ATOM | 2020 | OD1 | ASN | 252 | 7.166 | 38.626 | −4.587 | 1.00 | 9.37 |
| ATOM | 2021 | ND2 | ASN | 252 | 8.520 | 40.387 | −4.902 | 1.00 | 8.45 |
| ATOM | 2022 | C | ASN | 252 | 9.377 | 39.065 | −7.731 | 1.00 | 6.35 |
| ATOM | 2023 | O | ASN | 252 | 10.197 | 38.345 | −7.137 | 1.00 | 7.21 |
| ATOM | 2024 | N | CYS | 253 | 9.683 | 40.236 | −8.295 | 1.00 | 6.40 |
| ATOM | 2025 | CA | CYS | 253 | 11.048 | 40.768 | −8.285 | 1.00 | 7.03 |
| ATOM | 2026 | CB | CYS | 253 | 11.302 | 41.723 | −9.464 | 1.00 | 8.85 |
| ATOM | 2027 | SG | CYS | 253 | 11.232 | 40.881 | −11.077 | 1.00 | 10.36 |
| ATOM | 2028 | C | CYS | 253 | 11.309 | 41.552 | −6.988 | 1.00 | 7.30 |
| ATOM | 2029 | O | CYS | 253 | 10.402 | 42.203 | −6.420 | 1.00 | 7.55 |
| ATOM | 2030 | N | GLY | 254 | 12.562 | 41.539 | −6.585 | 1.00 | 6.67 |
| ATOM | 2031 | CA | GLY | 254 | 13.063 | 42.360 | −5.503 | 1.00 | 7.05 |
| ATOM | 2032 | C | GLY | 254 | 13.850 | 43.536 | −6.046 | 1.00 | 7.09 |
| ATOM | 2033 | O | GLY | 254 | 14.011 | 43.696 | −7.269 | 1.00 | 7.78 |
| ATOM | 2034 | N | SER | 255 | 14.338 | 44.380 | −5.130 | 1.00 | 7.39 |
| ATOM | 2035 | CA | SER | 255 | 14.942 | 45.634 | −5.560 | 1.00 | 8.33 |
| ATOM | 2036 | CB | SER | 255 | 14.980 | 46.646 | −4.420 | 1.00 | 8.09 |
| ATOM | 2037 | OG | SER | 255 | 15.785 | 46.124 | −3.385 | 1.00 | 9.54 |
| ATOM | 2038 | C | SER | 255 | 16.275 | 45.443 | −6.277 | 1.00 | 7.85 |
| ATOM | 2039 | O | SER | 255 | 16.710 | 46.348 | −6.979 | 1.00 | 10.10 |
| ATOM | 2040 | N | TYR | 256 | 16.928 | 44.287 | −6.184 | 1.00 | 7.83 |
| ATOM | 2041 | CA | TYR | 256 | 18.151 | 44.106 | −6.964 | 1.00 | 8.12 |
| ATOM | 2042 | CB | TYR | 256 | 18.966 | 42.909 | −6.486 | 1.00 | 7.96 |
| ATOM | 2043 | CG | TYR | 256 | 20.395 | 42.919 | −1.017 | 1.00 | 7.92 |
| ATOM | 2044 | CD1 | TYR | 256 | 21.351 | 43.735 | −6.425 | 1.00 | 9.18 |
| ATOM | 2045 | CE1 | TYR | 256 | 22.665 | 43.731 | −6.866 | 1.00 | 9.56 |
| ATOM | 2046 | CD2 | TYR | 256 | 20.800 | 42.110 | −8.068 | 1.00 | 8.38 |
| ATOM | 2047 | CE2 | TYR | 256 | 22.119 | 42.114 | −8.530 | 1.00 | 8.77 |
| ATOM | 2048 | CZ | TYR | 256 | 23.030 | 42.931 | −7.942 | 1.00 | 7.91 |
| ATOM | 2049 | OH | TYR | 256 | 24.334 | 42.919 | −8.446 | 1.00 | 10.55 |
| ATOM | 2050 | C | TYR | 256 | 17.790 | 44.007 | −8.444 | 1.00 | 7.77 |
| ATOM | 2051 | O | TYR | 256 | 18.510 | 44.559 | −9.307 | 1.00 | 9.14 |
| ATOM | 2052 | N | MET | 257 | 16.689 | 43.310 | −8.767 | 1.00 | 8.12 |
| ATOM | 2053 | CA | MET | 257 | 16.220 | 43.230 | −10.151 | 1.00 | 7.70 |
| ATOM | 2054 | CB | MET | 257 | 15.002 | 42.301 | −10.305 | 1.00 | 8.40 |
| ATOM | 2055 | CG | MET | 257 | 14.582 | 42.102 | −11.738 | 1.00 | 9.02 |
| ATOM | 2056 | SD | MET | 257 | 15.730 | 41.115 | −12.738 | 1.00 | 9.56 |
| ATOM | 2057 | CE | MET | 257 | 15.233 | 39.485 | −12.131 | 1.00 | 11.37 |
| ATOM | 2058 | C | MET | 257 | 15.843 | 44.629 | −10.670 | 1.00 | 6.94 |
| ATOM | 2059 | O | MET | 257 | 16.150 | 44.976 | −11.819 | 1.00 | 8.48 |
| ATOM | 2060 | N | ALA | 258 | 15.189 | 45.427 | −9.842 | 1.00 | 7.83 |
| ATOM | 2061 | CA | ALA | 258 | 14.807 | 46.786 | −10.242 | 1.00 | 8.49 |
| ATOM | 2062 | CB | ALA | 258 | 13.940 | 47.433 | −9.204 | 1.00 | 8.60 |
| ATOM | 2063 | C | ALA | 258 | 16.074 | 47.582 | −10.550 | 1.00 | 8.95 |
| ATOM | 2064 | O | ALA | 258 | 16.128 | 48.339 | −11.326 | 1.00 | 10.68 |
| ATOM | 2065 | N | HIS | 259 | 17.075 | 47.456 | −9.717 | 1.00 | 9.55 |
| ATOM | 2066 | CA | HIS | 259 | 18.325 | 48.184 | −9.987 | 1.00 | 9.84 |
| ATOM | 2067 | CB | HIS | 259 | 19.298 | 47.968 | −8.806 | 1.00 | 10.82 |
| ATOM | 2068 | CG | HIS | 259 | 20.581 | 48.672 | −8.960 | 1.00 | 12.01 |
| ATOM | 2069 | CD2 | HIS | 259 | 20.785 | 50.004 | −8.919 | 1.00 | 11.84 |
| ATOM | 2070 | ND1 | HIS | 259 | 21.801 | 48.079 | −9.209 | 1.00 | 14.52 |
| ATOM | 2071 | CE1 | HIS | 259 | 22.701 | 49.050 | −9.270 | 1.00 | 12.90 |
| ATOM | 2072 | NE2 | HIS | 259 | 22.106 | 50.209 | −9.115 | 1.00 | 14.66 |
| ATOM | 2073 | C | HIS | 259 | 18.949 | 47.746 | −11.296 | 1.00 | 9.17 |
| ATOM | 2074 | O | HIS | 259 | 19.275 | 48.580 | −12.144 | 1.00 | 11.20 |
| ATOM | 2075 | N | LEU | 260 | 19.091 | 46.450 | −11.529 | 1.00 | 9.71 |
| ATOM | 2076 | CA | LEU | 260 | 19.763 | 45.917 | −12.699 | 1.00 | 11.16 |
| ATOM | 2077 | CB | LEU | 260 | 19.771 | 44.362 | −12.647 | 1.00 | 13.64 |
| ATOM | 2078 | CG | LEU | 260 | 20.621 | 43.697 | −11.579 | 1.00 | 14.53 |
| ATOM | 2079 | CD1 | LEU | 260 | 20.446 | 42.195 | −11.659 | 1.00 | 15.33 |
| ATOM | 2080 | CD2 | LEU | 260 | 22.081 | 44.131 | −11.700 | 1.00 | 18.49 |
| ATOM | 2081 | C | LEU | 260 | 19.044 | 46.315 | −13.989 | 1.00 | 10.90 |
| ATOM | 2082 | O | LEU | 260 | 19.715 | 46.435 | −13.017 | 1.00 | 11.94 |
| ATOM | 2083 | N | THR | 261 | 17.723 | 46.442 | −13.932 | 1.00 | 9.82 |
| ATOM | 2084 | CA | THR | 261 | 16.920 | 46.680 | −15.141 | 1.00 | 10.23 |
| ATOM | 2085 | CB | THR | 261 | 15.671 | 45.798 | −15.202 | 1.00 | 9.32 |
| ATOM | 2086 | OG1 | THR | 261 | 14.737 | 46.138 | −14.174 | 1.00 | 9.59 |
| ATOM | 2087 | CG2 | THR | 261 | 16.000 | 44.325 | −15.098 | 1.00 | 10.96 |
| ATOM | 2088 | C | THR | 261 | 16.492 | 48.151 | −15.298 | 1.00 | 10.83 |
| ATOM | 2089 | O | THR | 261 | 15.664 | 48.493 | −16.138 | 1.00 | 12.22 |
| ATOM | 2090 | N | ASN | 262 | 17.009 | 48.999 | −14.417 | 1.00 | 12.86 |
| ATOM | 2091 | CA | ASN | 262 | 16.643 | 50.411 | −14.405 | 1.00 | 13.99 |
| ATOM | 2092 | CB | ASN | 262 | 17.162 | 51.124 | −15.670 | 1.00 | 16.72 |
| ATOM | 2093 | CG | ASN | 262 | 17.122 | 52.624 | −15.414 | 1.00 | 18.91 |
| ATOM | 2094 | OD1 | ASN | 262 | 17.484 | 53.032 | −14.306 | 1.00 | 23.20 |
| ATOM | 2095 | ND2 | ASN | 262 | 16.604 | 53.423 | −16.351 | 1.00 | 23.32 |
| ATOM | 2096 | C | ASN | 262 | 15.143 | 50.639 | −14.281 | 1.00 | 14.56 |
| ATOM | 2097 | O | ASN | 262 | 14.524 | 51.455 | −14.945 | 1.00 | 17.11 |
| ATOM | 2098 | N | ASN | 263 | 14.570 | 49.836 | −13.381 | 1.00 | 13.82 |
| ATOM | 2099 | CA | ASN | 263 | 13.148 | 49.902 | −13.081 | 1.00 | 15.46 |
| ATOM | 2100 | CB | ASN | 263 | 12.863 | 51.289 | −12.502 | 1.00 | 17.97 |
| ATOM | 2101 | CG | ASN | 263 | 12.214 | 51.276 | −11.157 | 1.00 | 17.06 |
| ATOM | 2102 | OD1 | ASN | 263 | 12.128 | 50.271 | −10.466 | 1.00 | 15.00 |
| ATOM | 2103 | ND2 | ASN | 263 | 11.533 | 52.359 | −10.866 | 1.00 | 25.64 |
| ATOM | 2104 | C | ASN | 263 | 12.252 | 49.450 | −14.215 | 1.00 | 13.77 |
| ATOM | 2105 | O | ASN | 263 | 11.012 | 49.566 | −14.160 | 1.00 | 18.41 |
| ATOM | 2106 | N | TYR | 264 | 12.774 | 48.764 | −15.236 | 1.00 | 13.79 |
| ATOM | 2107 | CA | TYR | 264 | 11.900 | 48.149 | −16.255 | 1.00 | 14.19 |
| ATOM | 2108 | CB | TYR | 264 | 12.715 | 47.613 | −17.416 | 1.00 | 15.13 |
| ATOM | 2109 | CG | TYR | 264 | 11.996 | 46.818 | −18.494 | 1.00 | 16.36 |
| ATOM | 2110 | CD1 | TYR | 264 | 11.035 | 47.371 | −19.362 | 1.00 | 17.78 |
| ATOM | 2111 | CE1 | TYR | 264 | 10.400 | 46.634 | −20.350 | 1.00 | 18.06 |
| ATOM | 2112 | CD2 | TYR | 264 | 12.259 | 45.484 | −18.745 | 1.00 | 16.68 |
| ATOM | 2113 | CE2 | TYR | 264 | 11.650 | 44.748 | −19.760 | 1.00 | 16.65 |
| ATOM | 2114 | CZ | TYR | 264 | 10.716 | 45.314 | −20 586 | 1.00 | 18.96 |
| ATOM | 2115 | OH | TYR | 264 | 10.037 | 44.587 | −21.573 | 1.00 | 18.75 |
| ATOM | 2116 | C | TYR | 264 | 11.078 | 47.051 | −15.593 | 1.00 | 12.40 |
| ATOM | 2117 | O | TYR | 264 | 9.880 | 46.958 | −15.851 | 1.00 | 13.61 |
| ATOM | 2118 | N | TYR | 265 | 11.724 | 46.245 | −14.738 | 1.00 | 10.62 |
| ATOM | 2119 | CA | TYR | 265 | 11.038 | 45.308 | −13.945 | 1.00 | 9.32 |
| ATOM | 2120 | CB | TYR | 265 | 11.580 | 43.896 | −13.877 | 1.00 | 9.65 |
| ATOM | 2121 | CG | TYR | 265 | 11.384 | 43.145 | −15.197 | 1.00 | 9.18 |
| ATOM | 2122 | CD1 | TYR | 265 | 11.979 | 41.897 | −15.374 | 1.00 | 9.12 |
| ATOM | 2123 | CE1 | TYR | 265 | 11.788 | 41.151 | −16.513 | 1.00 | 9.35 |
| ATOM | 2124 | CD2 | TYR | 265 | 10.575 | 43.607 | −16.232 | 1.00 | 9.97 |
| ATOM | 2125 | CE2 | TYR | 265 | 10.412 | 42.861 | −17.391 | 1.00 | 9.60 |
| ATOM | 2126 | CZ | TYR | 265 | 11.019 | 41.653 | −17.546 | 1.00 | 8.98 |
| ATOM | 2127 | OH | TYR | 265 | 10.956 | 40.929 | −18.694 | 1.00 | 9.24 |
| ATOM | 2128 | C | TYR | 265 | 11.143 | 45.898 | −12.455 | 1.00 | 8.87 |
| ATOM | 2129 | O | TYR | 265 | 12.165 | 45.736 | −11.793 | 1.00 | 10.25 |
| ATOM | 2130 | N | LYS | 266 | 10.091 | 46.591 | −12.039 | 1.00 | 9.35 |
| ATOM | 2131 | CA | LYS | 266 | 10.042 | 47.206 | −10.722 | 1.00 | 10.27 |
| ATOM | 2132 | CB | LYS | 266 | 8.764 | 48.067 | −10.642 | 1.00 | 11.78 |
| ATOM | 2133 | CG | LYS | 266 | 8.798 | 49.269 | −11.544 | 1.00 | 15.74 |
| ATOM | 2134 | CD | LYS | 266 | 7.679 | 50.219 | −11.609 | 1.00 | 20.41 |
| ATOM | 2135 | CE | LYS | 266 | 8.060 | 51.436 | −12.461 | 1.00 | 27.15 |
| ATOM | 2136 | NZ | LYS | 266 | 8.880 | 51.073 | −13.684 | 1.00 | 38.27 |
| ATOM | 2137 | C | LYS | 266 | 9.917 | 46.141 | −9.642 | 1.00 | 9.64 |
| ATOM | 2138 | O | LYS | 266 | 9.483 | 45.006 | −9.859 | 1.00 | 11.33 |
| ATOM | 2139 | N | ALA | 267 | 10.398 | 46.485 | −8.457 | 1.00 | 8.21 |
| ATOM | 2140 | CA | ALA | 267 | 10.177 | 45.669 | −7.302 | 1.00 | 8.00 |
| ATOM | 2141 | CB | ALA | 267 | 11.263 | 45.935 | −6.279 | 1.00 | 9.27 |
| ATOM | 2142 | C | ALA | 267 | 8.810 | 46.112 | −6.747 | 1.00 | 8.06 |
| ATOM | 2143 | O | ALA | 267 | 8.703 | 47.252 | −6.265 | 1.00 | 9.31 |
| ATOM | 2144 | N | PRO | 268 | 7.786 | 45.314 | −6.903 | 1.00 | 7.87 |
| ATOM | 2145 | CD | PRO | 268 | 7.704 | 43.960 | −7.464 | 1.00 | 8.45 |
| ATOM | 2146 | CA | PRO | 268 | 6.464 | 45.815 | −6.507 | 1.00 | 8.53 |
| ATOM | 2147 | CB | PRO | 268 | 5.497 | 44.732 | −6.971 | 1.00 | 8.95 |
| ATOM | 2148 | CG | PRO | 268 | 6.340 | 43.491 | −6.981 | 1.00 | 9.67 |
| ATOM | 2149 | C | PRO | 268 | 6.342 | 46.060 | −5.020 | 1.00 | 7.51 |
| ATOM | 2150 | O | PRO | 268 | 6.832 | 45.247 | −4.237 | 1.00 | 8.65 |
| ATOM | 2151 | N | ILE | 269 | 5.648 | 47.157 | −4.658 | 1.00 | 7.63 |
| ATOM | 2152 | CA | ILE | 269 | 5.356 | 47.441 | −3.254 | 1.00 | 7.94 |
| ATOM | 2153 | CB | ILE | 269 | 5.120 | 48.928 | −2.981 | 1.00 | 9.79 |
| ATOM | 2154 | CG2 | ILE | 269 | 4.494 | 49.172 | −1.634 | 1.00 | 10.56 |
| ATOM | 2155 | CG1 | ILE | 269 | 6.437 | 49.720 | −3.185 | 1.00 | 13.68 |
| ATOM | 2156 | CD1 | ILE | 269 | 6.187 | 51.182 | −3.479 | 1.00 | 23.32 |
| ATOM | 2157 | C | ILE | 269 | 4.170 | 46.593 | −2.837 | 1.00 | 7.83 |
| ATOM | 2158 | O | ILE | 269 | 3.149 | 46.540 | −3.511 | 1.00 | 9.16 |
| ATOM | 2159 | N | HIS | 270 | 4.317 | 45.894 | −1.703 | 1.00 | 7.29 |
| ATOM | 2160 | CA | HIS | 270 | 3.276 | 45.019 | −1.206 | 1.00 | 7.01 |
| ATOM | 2161 | CB | HIS | 270 | 3.400 | 43.626 | −1.835 | 1.00 | 7.25 |
| ATOM | 2162 | CG | HIS | 270 | 4.744 | 43.024 | −1.598 | 1.00 | 7.13 |
| ATOM | 2163 | CD2 | HIS | 270 | 5.128 | 42.038 | −0.746 | 1.00 | 7.31 |
| ATOM | 2164 | ND1 | HIS | 270 | 5.880 | 43.389 | −2.273 | 1.00 | 7.07 |
| ATOM | 2165 | CE1 | HIS | 270 | 6.904 | 42.683 | −1.826 | 1.00 | 7.87 |
| ATOM | 2166 | NE2 | HIS | 270 | 6.489 | 41.831 | −0.890 | 1.00 | 7.31 |
| ATOM | 2167 | C | HIS | 270 | 3.274 | 44.991 | 0.302 | 1.00 | 6.54 |
| ATOM | 2168 | O | HIS | 270 | 4.212 | 45.458 | 0.936 | 1.00 | 7.63 |
| ATOM | 2169 | N | ARG | 271 | 2.201 | 44.490 | 0.896 | 1.00 | 6.78 |
| ATOM | 2170 | CA | ARG | 271 | 1.988 | 44.467 | 2.318 | 1.00 | 6.60 |
| ATOM | 2171 | CB | ARG | 271 | 1.366 | 45.769 | 2.853 | 1.00 | 7.26 |
| ATOM | 2172 | CG | ARG | 271 | −0.088 | 45.972 | 2.398 | 1.00 | 8.12 |
| ATOM | 2173 | CD | ARG | 271 | −0.543 | 47.381 | 2.652 | 1.00 | 8.48 |
| ATOM | 2174 | NE | ARG | 271 | −1.974 | 47.504 | 2.297 | 1.00 | 9.27 |
| ATOM | 2175 | CZ | ARG | 271 | −2.643 | 48.651 | 2.312 | 1.00 | 9.64 |
| ATOM | 2176 | NH1 | ARG | 271 | −2.060 | 49.802 | 2.648 | 1.00 | 10.45 |
| ATOM | 2177 | NH2 | ARG | 271 | −3.930 | 48.660 | 1.968 | 1.00 | 11.63 |
| ATOM | 2178 | C | ARG | 271 | 1.144 | 43.252 | 2.682 | 1.00 | 6.79 |
| ATOM | 2179 | O | ARG | 271 | 0.517 | 42.637 | 1.805 | 1.00 | 7.95 |
| ATOM | 2180 | N | VAL | 272 | 1.154 | 42.887 | 3.953 | 1.00 | 7.39 |
| ATOM | 2181 | CA | VAL | 272 | 0.444 | 41.736 | 4.493 | 1.00 | 6.97 |
| ATOM | 2182 | CB | VAL | 272 | 1.369 | 40.830 | 5.328 | 1.00 | 7.37 |
| ATOM | 2183 | CG1 | VAL | 272 | 0.615 | 39.661 | 5.865 | 1.00 | 9.31 |
| ATOM | 2184 | CG2 | VAL | 272 | 2.609 | 40.388 | 4.562 | 1.00 | 9.68 |
| ATOM | 2185 | C | VAL | 272 | −0.712 | 42.173 | 5.378 | 1.00 | 6.78 |
| ATOM | 2186 | O | VAL | 272 | −0.470 | 42.805 | 6.415 | 1.00 | 7.67 |
| ATOM | 2187 | N | LYS | 273 | −1.917 | 41.839 | 4.950 | 1.00 | 7.33 |
| ATOM | 2188 | CA | LYS | 273 | −3.061 | 42.135 | 5.792 | 1.00 | 8.38 |
| ATOM | 2189 | CB | LYS | 273 | −4.364 | 41.763 | 5.047 | 1.00 | 9.72 |
| ATOM | 2190 | CG | LYS | 273 | −4.658 | 42.765 | 3.950 | 1.00 | 13.66 |
| ATOM | 2191 | CD | LYS | 273 | −6.090 | 42.725 | 3.437 | 1.00 | 20.08 |
| ATOM | 2192 | CE | LYS | 273 | −6.381 | 41.379 | 2.973 | 1.00 | 21.66 |
| ATOM | 2193 | NZ | LYS | 273 | −7.801 | 41.227 | 2.413 | 1.00 | 19.22 |
| ATOM | 2194 | C | LYS | 273 | −3.047 | 41.256 | 7.047 | 1.00 | 7.72 |
| ATOM | 2195 | O | LYS | 273 | −2.628 | 40.112 | 7.023 | 1.00 | 8.06 |
| ATOM | 2196 | N | TRP | 274 | −3.644 | 41.812 | 8.114 | 1.00 | 7.85 |
| ATOM | 2197 | CA | TRP | 274 | −3.914 | 41.041 | 9.322 | 1.00 | 8.01 |
| ATOM | 2198 | CB | TRP | 274 | −4.314 | 41.953 | 10.481 | 1.00 | 8.38 |
| ATOM | 2199 | CG | TRP | 274 | −4.864 | 41.210 | 11.655 | 1.00 | 8.22 |
| ATOM | 2200 | CD2 | TRP | 274 | −6.226 | 41.225 | 12.078 | 1.00 | 8.59 |
| ATOM | 2201 | CE2 | TRP | 274 | −6.311 | 40.370 | 13.188 | 1.00 | 9.28 |
| ATOM | 2202 | CE3 | TRP | 274 | −7.393 | 41.859 | 11.630 | 1.00 | 10.81 |
| ATOM | 2203 | CD1 | TRP | 274 | −4.216 | 40.377 | 12.490 | 1.00 | 8.98 |
| ATOM | 2204 | NE1 | TRP | 274 | −5.074 | 39.850 | 13.430 | 1.00 | 9.16 |
| ATOM | 2205 | CZ2 | TRP | 274 | −7.485 | 40.145 | 13.881 | 1.00 | 9.84 |
| ATOM | 2206 | CZ3 | TRP | 274 | −8.570 | 41.630 | 12.309 | 1.00 | 12.76 |
| ATOM | 2207 | CH2 | TRP | 274 | −8.591 | 40.765 | 13.403 | 1.00 | 12.58 |
| ATOM | 2208 | C | TRP | 274 | −5.114 | 40.123 | 9.043 | 1.00 | 7.32 |
| ATOM | 2209 | O | TRP | 274 | −6.156 | 40.621 | 8.598 | 1.00 | 8.37 |
| ATOM | 2210 | N | VAL | 275 | −4.963 | 38.854 | 9.345 | 1.00 | 7.40 |
| ATOM | 2211 | CA | VAL | 275 | −6.042 | 37.883 | 9.221 | 1.00 | 7.99 |
| ATOM | 2212 | CB | VAL | 275 | −5.913 | 37.057 | 7.931 | 1.00 | 11.04 |
| ATOM | 2213 | CG1 | VAL | 275 | −7.152 | 36.169 | 7.828 | 1.00 | 13.41 |
| ATOM | 2214 | CG2 | VAL | 275 | −5.765 | 37.904 | 6.702 | 1.00 | 15.17 |
| ATOM | 2215 | C | VAL | 275 | −5.933 | 37.000 | 10.457 | 1.00 | 7.32 |
| ATOM | 2216 | O | VAL | 275 | −4.818 | 36.547 | 10.759 | 1.00 | 8.66 |
| ATOM | 2217 | N | ASN | 276 | −7.013 | 36.774 | 11.175 | 1.00 | 7.50 |
| ATOM | 2218 | CA | ASN | 276 | −6.930 | 35.898 | 12.365 | 1.00 | 8.58 |
| ATOM | 2219 | CB | ASN | 276 | −7.921 | 36.377 | 13.414 | 1.00 | 9.69 |
| ATOM | 2220 | CG | ASN | 276 | −7.600 | 35.733 | 14.757 | 1.00 | 10.43 |
| ATOM | 2221 | OD1 | ASN | 276 | −6.631 | 34.956 | 14.885 | 1.00 | 9.69 |
| ATOM | 2222 | ND2 | ASN | 276 | −8.414 | 35.995 | 15.765 | 1.00 | 12.06 |
| ATOM | 2223 | C | ASN | 276 | −7.172 | 34.443 | 11.976 | 1.00 | 8.56 |
| ATOM | 2224 | O | ASN | 276 | −8.281 | 33.914 | 12.021 | 1.00 | 9.02 |
| ATOM | 2225 | N | ALA | 277 | −6.107 | 33.815 | 11.504 | 1.00 | 8.26 |
| ATOM | 2226 | CA | ALA | 277 | −6.107 | 32.466 | 10.982 | 1.00 | 8.04 |
| ATOM | 2227 | CB | ALA | 277 | −6.438 | 32.469 | 9.514 | 1.00 | 9.72 |
| ATOM | 2228 | C | ALA | 277 | −4.711 | 31.860 | 11.136 | 1.00 | 7.91 |
| ATOM | 2229 | O | ALA | 277 | −3.705 | 32.537 | 10.998 | 1.00 | 8.82 |
| ATOM | 2230 | N | GLU | 278 | −4.674 | 30.592 | 11.445 | 1.00 | 7.51 |
| ATOM | 2231 | CA | GLU | 278 | −3.460 | 29.801 | 11.557 | 1.00 | 7.98 |
| ATOM | 2232 | CB | GLU | 278 | −3.727 | 28.504 | 12.302 | 1.00 | 10.38 |
| ATOM | 2233 | CG | GLU | 278 | −2.539 | 27.650 | 12.655 | 1.00 | 11.75 |
| ATOM | 2234 | CD | GLU | 278 | −1.613 | 28.347 | 13.638 | 1.00 | 10.88 |
| ATOM | 2235 | OE1 | GLU | 278 | −0.419 | 28.424 | 13.314 | 1.00 | 12.63 |
| ATOM | 2236 | OE2 | GLU | 278 | −2.074 | 28.833 | 14.680 | 1.00 | 10.57 |
| ATOM | 2237 | C | GLU | 278 | −3.034 | 29.524 | 10.111 | 1.00 | 8.98 |
| ATOM | 2238 | O | GLU | 278 | −3.719 | 28.236 | 9.346 | 1.00 | 12.15 |
| ATOM | 2239 | N | ARG | 279 | −1.864 | 30.037 | 9.738 | 1.00 | 8.14 |
| ATOM | 2240 | CA | ARG | 279 | −1.410 | 29.901 | 8.373 | 1.00 | 8.39 |
| ATOM | 2241 | CB | ARG | 279 | −2.095 | 30.906 | 7.472 | 1.00 | 10.37 |
| ATOM | 2242 | CG | ARG | 279 | −1.659 | 32.310 | 7.818 | 1.00 | 10.01 |
| ATOM | 2243 | CD | ARG | 279 | −2.541 | 33.367 | 7.101 | 1.00 | 9.69 |
| ATOM | 2244 | NE | ARG | 279 | −2.026 | 34.712 | 7.405 | 1.00 | 9.29 |
| ATOM | 2245 | CZ | ARG | 279 | −0.965 | 35.208 | 6.775 | 1.00 | 9.20 |
| ATOM | 2246 | NH1 | ARG | 279 | −0.368 | 34.596 | 5.774 | 1.00 | 9.24 |
| ATOM | 2247 | NH2 | ARG | 279 | −0.490 | 36.396 | 7.129 | 1.00 | 9.29 |
| ATOM | 2248 | C | ARG | 279 | 0.095 | 29.920 | 8.275 | 1.00 | 7.13 |
| ATOM | 2249 | O | ARG | 279 | 0.821 | 30.149 | 9.252 | 1.00 | 8.10 |
| ATOM | 2250 | N | GLN | 280 | 0.593 | 29.601 | 7.095 | 1.00 | 7.21 |
| ATOM | 2251 | CA | GLN | 280 | 2.006 | 29.505 | 6.778 | 1.00 | 7.07 |
| ATOM | 2252 | CB | GLN | 280 | 2.318 | 28.088 | 6.255 | 1.00 | 8.87 |
| ATOM | 2253 | CG | GLN | 280 | 2.043 | 27.047 | 7.312 | 1.00 | 8.89 |
| ATOM | 2254 | CD | GLN | 280 | 2.188 | 25.645 | 6.771 | 1.00 | 8.58 |
| ATOM | 2255 | OE1 | GLN | 280 | 2.067 | 25.442 | 5.571 | 1.00 | 11.01 |
| ATOM | 2256 | NE2 | GLN | 280 | 2.533 | 24.689 | 7.591 | 1.00 | 8.97 |
| ATOM | 2257 | C | GLN | 280 | 2.389 | 30.454 | 5.653 | 1.00 | 6.21 |
| ATOM | 2258 | O | GLN | 280 | 1.634 | 30.606 | 4.671 | 1.00 | 7.53 |
| ATOM | 2259 | N | SER | 281 | 3.554 | 31.037 | 5.779 | 1.00 | 6.11 |
| ATOM | 2260 | CA | SER | 281 | 4.156 | 31.865 | 4.744 | 1.00 | 6.44 |
| ATOM | 2261 | CB | SER | 281 | 4.016 | 33.345 | 5.109 | 1.00 | 7.40 |
| ATOM | 2262 | OG | SER | 281 | 4.520 | 34.121 | 4.016 | 1.00 | 8.04 |
| ATOM | 2263 | C | SER | 281 | 5.617 | 31.443 | 4.606 | 1.00 | 6.56 |
| ATOM | 2264 | O | SER | 281 | 6.374 | 31.574 | 5.574 | 1.00 | 6.58 |
| ATOM | 2265 | N | LEU | 282 | 5.952 | 30.778 | 3.493 | 1.00 | 5.40 |
| ATOM | 2266 | CA | LEU | 282 | 7.243 | 30.105 | 3.352 | 1.00 | 6.03 |
| ATOM | 2267 | CB | LEU | 282 | 6.986 | 28.583 | 3.195 | 1.00 | 6.16 |
| ATOM | 2268 | CG | LEU | 282 | 5.982 | 27.942 | 4.203 | 1.00 | 7.21 |
| ATOM | 2269 | CD1 | LEU | 282 | 5.851 | 26.441 | 3.942 | 1.00 | 8.79 |
| ATOM | 2270 | CD2 | LEU | 282 | 6.340 | 28.188 | 5.661 | 1.00 | 8.29 |
| ATOM | 2271 | C | LEU | 282 | 8.025 | 30.641 | 2.173 | 1.00 | 5.52 |
| ATOM | 2272 | O | LEU | 282 | 8.055 | 30.039 | 1.071 | 1.00 | 5.66 |
| ATOM | 2273 | N | PRO | 283 | 8.632 | 31.801 | 2.318 | 1.00 | 5.55 |
| ATOM | 2274 | CD | PRO | 283 | 8.549 | 32.739 | 3.463 | 1.00 | 5.92 |
| ATOM | 2275 | CA | PRO | 283 | 9.437 | 32.349 | 1.222 | 1.00 | 5.72 |
| ATOM | 2276 | CB | PRO | 283 | 9.634 | 33.815 | 1.631 | 1.00 | 6.44 |
| ATOM | 2277 | CG | PRO | 283 | 9.624 | 33.748 | 3.131 | 1.00 | 6.28 |
| ATOM | 2278 | C | PRO | 283 | 10.795 | 31.729 | 1.057 | 1.00 | 4.97 |
| ATOM | 2279 | O | PRO | 283 | 11.445 | 31.312 | 2.006 | 1.00 | 5.92 |
| ATOM | 2280 | N | PHE | 284 | 11.262 | 31.720 | −0.187 | 1.00 | 5.22 |
| ATOM | 2281 | CA | PHE | 284 | 12.642 | 31.498 | −0.602 | 1.00 | 4.80 |
| ATOM | 2282 | CB | PHE | 284 | 12.785 | 30.323 | −1.566 | 1.00 | 5.14 |
| ATOM | 2283 | CG | PHE | 284 | 14.184 | 29.960 | −1.983 | 1.00 | 4.79 |
| ATOM | 2284 | CD1 | PHE | 284 | 15.089 | 29.473 | −1.057 | 1.00 | 5.39 |
| ATOM | 2285 | CD2 | PHE | 284 | 14.591 | 30.026 | −3.286 | 1.00 | 5.88 |
| ATOM | 2286 | CE1 | PHE | 284 | 16.341 | 29.032 | −1.451 | 1.00 | 5.76 |
| ATOM | 2287 | CE2 | PHE | 284 | 15.828 | 29.633 | −3.714 | 1.00 | 5.70 |
| ATOM | 2288 | CZ | PHE | 284 | 16.714 | 29.109 | −2.787 | 1.00 | 5.57 |
| ATOM | 2289 | C | PHE | 284 | 13.119 | 32.795 | −1.263 | 1.00 | 4.78 |
| ATOM | 2290 | O | PHE | 284 | 12.566 | 33.190 | −2.308 | 1.00 | 5.98 |
| ATOM | 2291 | N | PHE | 285 | 14.114 | 33.448 | −0.659 | 1.00 | 5.01 |
| ATOM | 2292 | CA | PHE | 285 | 14.656 | 34.687 | −1.215 | 1.00 | 5.10 |
| ATOM | 2293 | CB | PHE | 285 | 15.058 | 35.637 | −0.062 | 1.00 | 6.13 |
| ATOM | 2294 | CG | PHE | 285 | 13.858 | 36.096 | 0.747 | 1.00 | 6.38 |
| ATOM | 2295 | CD1 | PHE | 285 | 13.665 | 35.656 | 2.044 | 1.00 | 6.54 |
| ATOM | 2296 | CD2 | PHE | 285 | 12.909 | 36.933 | 0.195 | 1.00 | 6.45 |
| ATOM | 2297 | CE1 | PHE | 285 | 12.534 | 36.014 | 2.765 | 1.00 | 7.21 |
| ATOM | 2298 | CE2 | PHE | 285 | 11.781 | 37.303 | 0.891 | 1.00 | 7.45 |
| ATOM | 2299 | CZ | PHE | 285 | 11.610 | 36.853 | 2.177 | 1.00 | 7.71 |
| ATOM | 2300 | C | PHE | 285 | 15.812 | 34.333 | −2.108 | 1.00 | 5.08 |
| ATOM | 2301 | O | PHE | 285 | 16.815 | 33.800 | −1.608 | 1.00 | 6.02 |
| ATOM | 2302 | N | VAL | 286 | 15.658 | 34.588 | −3.399 | 1.00 | 5.58 |
| ATOM | 2303 | CA | VAL | 286 | 16.696 | 34.255 | −4.364 | 1.00 | 5.56 |
| ATOM | 2304 | CB | VAL | 286 | 16.104 | 34.056 | −5.766 | 1.00 | 5.78 |
| ATOM | 2305 | CG1 | VAL | 286 | 17.189 | 33.783 | −6.814 | 1.00 | 7.42 |
| ATOM | 2306 | CG2 | VAL | 286 | 15.065 | 32.922 | −5.710 | 1.00 | 7.19 |
| ATOM | 2307 | C | VAL | 286 | 17.756 | 35.372 | −4.347 | 1.00 | 5.98 |
| ATOM | 2308 | O | VAL | 286 | 17.569 | 36.453 | −4.895 | 1.00 | 6.92 |
| ATOM | 2309 | N | ASN | 287 | 18.839 | 35.082 | −3.642 | 1.00 | 5.82 |
| ATOM | 2310 | CA | ASN | 287 | 20.008 | 35.922 | −3.466 | 1.00 | 6.12 |
| ATOM | 2311 | CB | ASN | 287 | 20.324 | 36.042 | −1.971 | 1.00 | 6.40 |
| ATOM | 2312 | CG | ASN | 287 | 19.235 | 36.737 | −1.183 | 1.00 | 6.67 |
| ATOM | 2313 | OD1 | ASN | 287 | 18.623 | 37.682 | −1.683 | 1.00 | 7.35 |
| ATOM | 2314 | ND2 | ASN | 287 | 19.036 | 36.262 | 0.073 | 1.00 | 6.70 |
| ATOM | 2315 | C | ASN | 287 | 21.187 | 35.296 | −4.216 | 1.00 | 5.72 |
| ATOM | 2316 | O | ASN | 287 | 21.271 | 34.072 | −4.300 | 1.00 | 6.46 |
| ATOM | 2317 | N | LEU | 288 | 22.049 | 36.145 | −4.781 | 1.00 | 6.71 |
| ATOM | 2318 | CA | LEU | 288 | 23.223 | 35.679 | −5.499 | 1.00 | 6.46 |
| ATOM | 2319 | CB | LEU | 288 | 23.538 | 36.703 | −6.595 | 1.00 | 7.57 |
| ATOM | 2320 | CG | LEU | 288 | 22.371 | 36.977 | −7.570 | 1.00 | 8.20 |
| ATOM | 2321 | CD1 | LEU | 288 | 22.819 | 37.998 | −8.621 | 1.00 | 10.08 |
| ATOM | 2322 | CD2 | LEU | 288 | 21.880 | 35.687 | −8.189 | 1.00 | 8.32 |
| ATOM | 2323 | C | LEU | 288 | 24.381 | 35.444 | −4.525 | 1.00 | 7.33 |
| ATOM | 2324 | O | LEU | 288 | 24.140 | 35.277 | −3.317 | 1.00 | 8.58 |
| ATOM | 2325 | N | GLY | 289 | 25.607 | 35.358 | −5.037 | 1.00 | 7.93 |
| ATOM | 2326 | CA | GLY | 289 | 26.765 | 35.216 | −4.155 | 1.00 | 7.86 |
| ATOM | 2327 | C | GLY | 289 | 27.242 | 36.571 | −3.667 | 1.00 | 7.24 |
| ATOM | 2328 | O | GLY | 289 | 26.890 | 37.634 | −4.186 | 1.00 | 8.44 |
| ATOM | 2329 | N | TYR | 290 | 28.075 | 36.559 | −2.633 | 1.00 | 7.71 |
| ATOM | 2330 | CA | TYR | 290 | 28.503 | 37.789 | −1.986 | 1.00 | 8.91 |
| ATOM | 2331 | CB | TYR | 290 | 29.407 | 37.453 | −0.813 | 1.00 | 9.56 |
| ATOM | 2332 | CG | TYR | 290 | 29.638 | 38.614 | 0.135 | 1.00 | 9.12 |
| ATOM | 2333 | CD1 | TYR | 290 | 28.720 | 38.961 | 1.118 | 1.00 | 10.55 |
| ATOM | 2334 | CE1 | TYR | 290 | 28.949 | 40.041 | 1.966 | 1.00 | 12.13 |
| ATOM | 2335 | CD2 | TYR | 290 | 30.792 | 39.367 | 0.036 | 1.00 | 12.09 |
| ATOM | 2336 | CE2 | TYR | 290 | 31.035 | 40.426 | 0.887 | 1.00 | 13.43 |
| ATOM | 2337 | CZ | TYR | 290 | 30.129 | 40.766 | 1.861 | 1.00 | 13.85 |
| ATOM | 2338 | OH | TYR | 290 | 30.407 | 41.847 | 2.679 | 1.00 | 16.61 |
| ATOM | 2339 | C | TYR | 290 | 29.196 | 38.783 | −2.903 | 1.00 | 8.98 |
| ATOM | 2340 | O | TYR | 290 | 28.979 | 39.980 | −2.814 | 1.00 | 9.66 |
| ATOM | 2341 | N | ASP | 291 | 30.009 | 38.246 | −3.816 | 1.00 | 9.63 |
| ATOM | 2342 | CA | ASP | 291 | 30.738 | 39.081 | −4.766 | 1.00 | 11.13 |
| ATOM | 2343 | CB | ASP | 291 | 32.143 | 38.545 | −4.920 | 1.00 | 14.78 |
| ATOM | 2344 | CG | ASP | 291 | 33.015 | 38.830 | −3.715 | 1.00 | 19.43 |
| ATOM | 2345 | OD1 | ASP | 291 | 34.005 | 38.109 | −3.515 | 1.00 | 30.38 |
| ATOM | 2346 | OD2 | ASP | 291 | 32.776 | 39.787 | −2.963 | 1.00 | 21.52 |
| ATOM | 2347 | C | ASP | 291 | 30.075 | 39.198 | −6.123 | 1.00 | 10.76 |
| ATOM | 2348 | O | ASP | 291 | 30.676 | 39.770 | −7.035 | 1.00 | 14.21 |
| ATOM | 2349 | N | SER | 292 | 28.867 | 38.636 | −6.294 | 1.00 | 10.05 |
| ATOM | 2350 | CA | SER | 292 | 28.226 | 38.714 | −7.606 | 1.00 | 9.39 |
| ATOM | 2351 | CB | SER | 292 | 26.908 | 37.921 | −7.562 | 1.00 | 9.60 |
| ATOM | 2352 | OG | SER | 292 | 27.117 | 36.538 | −7.241 | 1.00 | 10.02 |
| ATOM | 2353 | C | SER | 292 | 27.915 | 40.181 | −7.922 | 1.00 | 9.65 |
| ATOM | 2354 | O | SER | 292 | 27.303 | 40.934 | −7.170 | 1.00 | 10.11 |
| ATOM | 2355 | N | VAL | 293 | 28.284 | 40.587 | −9.123 | 1.00 | 11.44 |
| ATOM | 2356 | CA | VAL | 293 | 27.948 | 41.879 | −9.675 | 1.00 | 12.30 |
| ATOM | 2357 | CB | VAL | 293 | 29.177 | 42.809 | −9.749 | 1.00 | 14.95 |
| ATOM | 2358 | CG1 | VAL | 293 | 28.691 | 44.199 | −10.166 | 1.00 | 20.38 |
| ATOM | 2359 | CG2 | VAL | 293 | 29.947 | 42.902 | −8.464 | 1.00 | 18.62 |
| ATOM | 2360 | C | VAL | 293 | 27.402 | 41.683 | −11.091 | 1.00 | 13.15 |
| ATOM | 2361 | O | VAL | 293 | 27.976 | 41.020 | −11.971 | 1.00 | 17.31 |
| ATOM | 2362 | N | ILE | 294 | 26.232 | 42.246 | −11.309 | 1.00 | 13.36 |
| ATOM | 2363 | CA | ILE | 294 | 25.669 | 42.289 | −12.650 | 1.00 | 16.26 |
| ATOM | 2364 | CB | ILE | 294 | 24.283 | 41.645 | −12.648 | 1.00 | 18.21 |
| ATOM | 2365 | CG2 | ILE | 294 | 23.540 | 41.879 | −13.941 | 1.00 | 22.79 |
| ATOM | 2366 | CG1 | ILE | 294 | 24.529 | 40.135 | −12.350 | 1.00 | 19.50 |
| ATOM | 2367 | CD1 | ILE | 294 | 23.255 | 39.371 | −12.397 | 1.00 | 21.00 |
| ATOM | 2368 | C | ILE | 294 | 25.616 | 43.756 | −13.059 | 1.00 | 14.95 |
| ATOM | 2369 | O | ILE | 294 | 25.193 | 44.619 | −12.311 | 1.00 | 14.96 |
| ATOM | 2370 | N | ASP | 295 | 26.164 | 44.020 | −14.235 | 1.00 | 15.39 |
| ATOM | 2371 | CA | ASP | 295 | 26.203 | 45.398 | −14.709 | 1.00 | 16.58 |
| ATOM | 2372 | CB | ASP | 295 | 27.214 | 45.501 | −15.857 | 1.00 | 23.82 |
| ATOM | 2373 | CG | ASP | 295 | 28.612 | 45.188 | −15.298 | 1.00 | 30.17 |
| ATOM | 2374 | OD1 | ASP | 295 | 29.304 | 44.457 | −16.044 | 1.00 | 42.83 |
| ATOM | 2375 | OD2 | ASP | 295 | 29.000 | 45.638 | −14.184 | 1.00 | 32.91 |
| ATOM | 2376 | C | ASP | 295 | 24.803 | 45.841 | −15.109 | 1.00 | 13.64 |
| ATOM | 2377 | O | ASP | 295 | 24.072 | 45.199 | −15.856 | 1.00 | 14.07 |
| ATOM | 2378 | N | PRO | 296 | 24.390 | 46.969 | −14.533 | 1.00 | 12.78 |
| ATOM | 2379 | CD | PRO | 296 | 25.049 | 47.684 | −13.426 | 1.00 | 14.44 |
| ATOM | 2380 | CA | PRO | 296 | 23.029 | 47.467 | −14.847 | 1.00 | 12.67 |
| ATOM | 2381 | CB | PRO | 296 | 22.877 | 48.659 | −13.923 | 1.00 | 14.23 |
| ATOM | 2382 | CG | PRO | 296 | 23.840 | 48.351 | −12.818 | 1.00 | 16.25 |
| ATOM | 2383 | C | PRO | 296 | 22.825 | 47.803 | −16.319 | 1.00 | 12.63 |
| ATOM | 2384 | O | PRO | 296 | 23.750 | 48.221 | −17.032 | 1.00 | 14.48 |
| ATOM | 2385 | N | PHE | 297 | 21.602 | 47.615 | −16.800 | 1.00 | 11.48 |
| ATOM | 2386 | CA | PHE | 297 | 21.260 | 47.872 | −18.195 | 1.00 | 11.96 |
| ATOM | 2387 | CB | PHE | 297 | 21.388 | 46.632 | −19.046 | 1.00 | 12.04 |
| ATOM | 2388 | CG | PHE | 297 | 20.543 | 45.425 | −18.690 | 1.00 | 12.30 |
| ATOM | 2389 | CD1 | PHE | 297 | 19.390 | 45.126 | −19.386 | 1.00 | 15.33 |
| ATOM | 2390 | CD2 | PHE | 297 | 20.900 | 44.597 | −17.664 | 1.00 | 12.58 |
| ATOM | 2391 | CE1 | PHE | 297 | 18.648 | 44.005 | −19.091 | 1.00 | 17.14 |
| ATOM | 2392 | CE2 | PHE | 297 | 20.121 | 43.510 | −17.311 | 1.00 | 14.52 |
| ATOM | 2393 | CZ | PHE | 297 | 18.969 | 43.235 | −18.003 | 1.00 | 16.56 |
| ATOM | 2394 | C | PHE | 297 | 19.837 | 48.421 | −18.246 | 1.00 | 13.07 |
| ATOM | 2395 | O | PHE | 297 | 19.162 | 48.472 | −17.219 | 1.00 | 14.15 |
| ATOM | 2396 | N | ASP | 298 | 19.390 | 48.809 | −19.429 | 1.00 | 16.06 |
| ATOM | 2397 | CA | ASP | 298 | 18.024 | 49.272 | −19.601 | 1.00 | 15.45 |
| ATOM | 2398 | CB | ASP | 298 | 18.023 | 50.788 | −19.547 | 1.00 | 17.44 |
| ATOM | 2399 | CG | ASP | 298 | 16.519 | 51.374 | −19.488 | 1.00 | 17.86 |
| ATOM | 2400 | OD1 | ASP | 298 | 15.634 | 50.717 | −19.809 | 1.00 | 17.50 |
| ATOM | 2401 | OD2 | ASP | 298 | 16.580 | 52.580 | −19.143 | 1.00 | 20.86 |
| ATOM | 2402 | C | ASP | 298 | 17.436 | 48.753 | −20.900 | 1.00 | 16.52 |
| ATOM | 2403 | O | ASP | 298 | 17.736 | 49.281 | −21.973 | 1.00 | 17.05 |
| ATOM | 2404 | N | PRO | 299 | 16.541 | 47.776 | −20.829 | 1.00 | 16.19 |
| ATOM | 2405 | CD | PRO | 299 | 16.199 | 47.050 | −19.589 | 1.00 | 17.38 |
| ATOM | 2406 | CA | PRO | 299 | 15.887 | 47.212 | −22.014 | 1.00 | 16.63 |
| ATOM | 2407 | CB | PRO | 299 | 15.397 | 45.853 | −21.561 | 1.00 | 19.69 |
| ATOM | 2408 | CG | PRO | 299 | 15.676 | 45.745 | −20.112 | 1.00 | 21.06 |
| ATOM | 2409 | C | PRO | 299 | 14.854 | 48.081 | −22.726 | 1.00 | 17.45 |
| ATOM | 2410 | O | PRO | 299 | 14.213 | 47.662 | −23.730 | 1.00 | 18.54 |
| ATOM | 2411 | N | ARG | 300 | 14.703 | 49.327 | −22.232 | 1.00 | 16.34 |
| ATOM | 2412 | CA | ARG | 300 | 13.857 | 50.321 | −22.881 | 1.00 | 18.12 |
| ATOM | 2413 | CB | ARG | 300 | 13.086 | 51.146 | −21.833 | 1.00 | 19.33 |
| ATOM | 2414 | CG | ARG | 300 | 11.959 | 50.301 | −21.242 | 1.00 | 21.93 |
| ATOM | 2415 | CD | ARG | 300 | 11.237 | 50.861 | −20.020 | 1.00 | 25.26 |
| ATOM | 2416 | NE | ARG | 300 | 12.231 | 51.198 | −19.046 | 1.00 | 30.85 |
| ATOM | 2417 | CZ | ARG | 300 | 12.419 | 51.701 | −17.857 | 1.00 | 29.50 |
| ATOM | 2418 | NH1 | ARG | 300 | 11.422 | 52.069 | −17.096 | 1.00 | 32.95 |
| ATOM | 2419 | NH2 | ARG | 300 | 13.671 | 51.781 | −17.407 | 1.00 | 30.69 |
| ATOM | 2420 | C | ARG | 300 | 14.691 | 51.279 | −23.726 | 1.00 | 17.96 |
| ATOM | 2421 | O | ARG | 300 | 14.138 | 52.081 | −24.462 | 1.00 | 17.01 |
| ATOM | 2422 | N | GLU | 301 | 15.997 | 51.254 | −23.552 | 1.00 | 17.71 |
| ATOM | 2423 | CA | GLU | 301 | 16.913 | 52.185 | −24.206 | 1.00 | 19.38 |
| ATOM | 2424 | C | GLU | 301 | 17.619 | 51.524 | −25.392 | 1.00 | 18.52 |
| ATOM | 2425 | O | GLU | 301 | 18.127 | 50.400 | −25.261 | 1.00 | 17.52 |
| ATOM | 2426 | CB | GLU | 301 | 17.949 | 52.595 | −23.157 | 1.00 | 23.66 |
| ATOM | 2427 | CG | GLU | 301 | 17.521 | 53.757 | −22.322 | 1.00 | 27.35 |
| ATOM | 2428 | CD | GLU | 301 | 16.969 | 54.959 | −23.059 | 1.00 | 32.53 |
| ATOM | 2429 | OE1 | GLU | 301 | 17.705 | 55.499 | −23.901 | 1.00 | 38.75 |
| ATOM | 2430 | OE2 | GLU | 301 | 15.841 | 55.411 | −22.764 | 1.00 | 38.29 |
| ATOM | 2431 | N | PRO | 302 | 17.667 | 52.160 | −26.553 | 1.00 | 18.63 |
| ATOM | 2432 | CD | PRO | 302 | 17.042 | 53.464 | −26.839 | 1.00 | 21.36 |
| ATOM | 2433 | CA | PRO | 302 | 18.277 | 51.571 | −27.738 | 1.00 | 20.96 |
| ATOM | 2434 | CB | PRO | 302 | 18.222 | 52.693 | −28.776 | 1.00 | 22.61 |
| ATOM | 2435 | CG | PRO | 302 | 17.001 | 53.446 | −28.341 | 1.00 | 22.90 |
| ATOM | 2436 | C | PRO | 302 | 19.683 | 51.057 | −27.579 | 1.00 | 21.62 |
| ATOM | 2437 | O | PRO | 302 | 19.874 | 49.926 | −28.009 | 1.00 | 24.93 |
| ATOM | 2438 | N | ASN | 303 | 20.520 | 51.712 | −26.771 | 1.00 | 21.61 |
| ATOM | 2439 | CA | ASN | 303 | 21.840 | 51.125 | −26.582 | 1.00 | 22.90 |
| ATOM | 2440 | CB | ASN | 303 | 22.905 | 52.194 | −26.448 | 1.00 | 26.88 |
| ATOM | 2441 | CG | ASN | 303 | 22.642 | 53.204 | −25.353 | 1.00 | 27.57 |
| ATOM | 2442 | OD1 | ASN | 303 | 21.822 | 52.996 | −24.467 | 1.00 | 23.80 |
| ATOM | 2443 | ND2 | ASN | 303 | 23.353 | 54.329 | −25.453 | 1.00 | 29.21 |
| ATOM | 2444 | C | ASN | 303 | 21.931 | 50.221 | −25.360 | 1.00 | 20.67 |
| ATOM | 2445 | O | ASN | 303 | 23.039 | 49.773 | −25.049 | 1.00 | 23.78 |
| ATOM | 2446 | N | GLY | 304 | 20.847 | 49.989 | −24.661 | 1.00 | 17.93 |
| ATOM | 2447 | CA | GLY | 304 | 20.793 | 49.190 | −23.443 | 1.00 | 18.03 |
| ATOM | 2448 | C | GLY | 304 | 21.488 | 49.856 | −22.276 | 1.00 | 17.52 |
| ATOM | 2449 | O | GLY | 304 | 21.576 | 49.239 | −21.215 | 1.00 | 20.50 |
| ATOM | 2450 | N | LYS | 305 | 22.009 | 51.071 | −22.342 | 1.00 | 18.09 |
| ATOM | 2451 | CA | LYS | 305 | 22.768 | 51.590 | −21.208 | 1.00 | 20.79 |
| ATOM | 2452 | C | LYS | 305 | 21.921 | 52.252 | −20.147 | 1.00 | 20.39 |
| ATOM | 2453 | O | LYS | 305 | 20.850 | 52.777 | −20.392 | 1.00 | 21.26 |
| ATOM | 2454 | CB | LYS | 305 | 23.884 | 52.524 | −21.675 | 1.00 | 24.72 |
| ATOM | 2455 | CG | LYS | 305 | 25.002 | 51.789 | −22.421 | 1.00 | 30.61 |
| ATOM | 2456 | CD | LYS | 305 | 26.032 | 52.816 | −22.869 | 1.00 | 36.33 |
| ATOM | 2457 | CE | LYS | 305 | 26.246 | 52.908 | −24.360 | 1.00 | 39.54 |
| ATOM | 2458 | NZ | LYS | 305 | 27.649 | 52.574 | −24.760 | 1.00 | 51.21 |
| ATOM | 2459 | N | SER | 306 | 22.461 | 52.202 | −18.921 | 1.00 | 18.96 |
| ATOM | 2460 | CA | SER | 306 | 21.758 | 52.837 | −17.816 | 1.00 | 19.30 |
| ATOM | 2461 | CB | SER | 306 | 21.210 | 51.732 | −16.911 | 1.00 | 23.77 |
| ATOM | 2462 | OG | SER | 306 | 21.758 | 51.769 | −15.640 | 1.00 | 26.38 |
| ATOM | 2463 | C | SER | 306 | 22.711 | 53.732 | −17.050 | 1.00 | 16.95 |
| ATOM | 2464 | O | SER | 306 | 23.919 | 53.528 | −16.996 | 1.00 | 20.29 |
| ATOM | 2465 | N | ASP | 307 | 22.159 | 54.728 | −16.365 | 1.00 | 18.07 |
| ATOM | 2466 | CA | ASP | 307 | 22.902 | 55.647 | −15.527 | 1.00 | 17.75 |
| ATOM | 2467 | CB | ASP | 307 | 22.343 | 57.066 | −15.646 | 1.00 | 19.69 |
| ATOM | 2468 | CG | ASP | 307 | 22.554 | 57.644 | −17.030 | 1.00 | 21.31 |
| ATOM | 2469 | OD1 | ASP | 307 | 23.544 | 57.289 | −17.717 | 1.00 | 25.76 |
| ATOM | 2470 | OD2 | ASP | 307 | 21.697 | 58.413 | −17.492 | 1.00 | 22.42 |
| ATOM | 2471 | C | ASP | 307 | 23.003 | 55.194 | −14.083 | 1.00 | 17.12 |
| ATOM | 2472 | O | ASP | 307 | 23.267 | 55.998 | −13.150 | 1.00 | 15.97 |
| ATOM | 2473 | N | ARG | 308 | 22.897 | 53.886 | −13.877 | 1.00 | 18.84 |
| ATOM | 2474 | CA | ARG | 308 | 23.109 | 53.313 | −12.542 | 1.00 | 17.51 |
| ATOM | 2475 | CB | ARG | 308 | 22.067 | 52.271 | −12.242 | 1.00 | 16.86 |
| ATOM | 2476 | CG | ARG | 308 | 20.688 | 52.745 | −11.848 | 1.00 | 15.69 |
| ATOM | 2477 | CD | ARG | 308 | 19.627 | 51.694 | −11.982 | 1.00 | 18.04 |
| ATOM | 2478 | NE | ARG | 308 | 18.312 | 52.224 | −11.687 | 1.00 | 17.41 |
| ATOM | 2479 | CZ | ARG | 308 | 17.733 | 52.219 | −10.487 | 1.00 | 15.39 |
| ATOM | 2480 | NH1 | ARG | 308 | 18.288 | 51.740 | −9.404 | 1.00 | 14.58 |
| ATOM | 2481 | NH2 | ARG | 308 | 16.517 | 52.713 | −10.426 | 1.00 | 19.03 |
| ATOM | 2482 | C | ARG | 308 | 24.511 | 52.680 | −12.443 | 1.00 | 18.59 |
| ATOM | 2483 | O | ARG | 308 | 25.055 | 52.218 | −13.440 | 1.00 | 25.55 |
| ATOM | 2484 | N | GLU | 309 | 25.070 | 52.659 | −11.241 | 1.00 | 18.16 |
| ATOM | 2485 | CA | GLU | 309 | 26.350 | 52.054 | −10.946 | 1.00 | 19.38 |
| ATOM | 2486 | CB | GLU | 309 | 27.058 | 52.935 | −9.907 | 1.00 | 21.98 |
| ATOM | 2487 | CG | GLU | 309 | 27.442 | 54.299 | −10.501 | 1.00 | 25.52 |
| ATOM | 2488 | CD | GLU | 309 | 28.319 | 54.244 | −11.731 | 1.00 | 30.05 |
| ATOM | 2489 | OE1 | GLU | 309 | 29.244 | 53.401 | −11.842 | 1.00 | 36.16 |
| ATOM | 2490 | OE2 | GLU | 309 | 28.114 | 55.058 | −12.663 | 1.00 | 37.66 |
| ATOM | 2491 | C | GLU | 309 | 26.233 | 50.607 | −10.484 | 1.00 | 15.87 |
| ATOM | 2492 | O | GLU | 309 | 25.287 | 50.180 | −9.826 | 1.00 | 14.89 |
| ATOM | 2493 | N | PRO | 310 | 27.164 | 49.735 | −10.842 | 1.00 | 14.73 |
| ATOM | 2494 | CD | PRO | 310 | 28.275 | 50.012 | −11.799 | 1.00 | 16.91 |
| ATOM | 2495 | CA | PRO | 310 | 27.152 | 48.348 | −10.386 | 1.00 | 15.04 |
| ATOM | 2496 | CB | PRO | 310 | 28.419 | 47.733 | −11.059 | 1.00 | 17.03 |
| ATOM | 2497 | CG | PRO | 310 | 28.700 | 48.625 | −12.220 | 1.00 | 19.09 |
| ATOM | 2498 | C | PRO | 310 | 27.219 | 48.266 | −8.874 | 1.00 | 13.88 |
| ATOM | 2499 | O | PRO | 310 | 27.841 | 49.068 | −8.164 | 1.00 | 15.60 |
| ATOM | 2500 | N | LEU | 311 | 26.474 | 47.318 | −8.360 | 1.00 | 12.14 |
| ATOM | 2501 | CA | LEU | 311 | 26.338 | 47.085 | −6.922 | 1.00 | 11.25 |
| ATOM | 2502 | CB | LEU | 311 | 24.878 | 47.460 | −6.615 | 1.00 | 13.56 |
| ATOM | 2503 | CG | LEU | 311 | 24.457 | 47.333 | −5.180 | 1.00 | 13.90 |
| ATOM | 2504 | CD1 | LEU | 311 | 25.339 | 48.120 | −4.233 | 1.00 | 20.42 |
| ATOM | 2505 | CD2 | LEU | 311 | 22.986 | 47.700 | −5.047 | 1.00 | 17.32 |
| ATOM | 2506 | C | LEU | 311 | 26.572 | 45.604 | −6.622 | 1.00 | 10.64 |
| ATOM | 2507 | O | LEU | 311 | 25.915 | 44.759 | −7.247 | 1.00 | 11.74 |
| ATOM | 2508 | N | SER | 312 | 27.494 | 45.257 | −5.728 | 1.00 | 10.37 |
| ATOM | 2509 | CA | SER | 312 | 27.725 | 43.854 | −5.407 | 1.00 | 11.32 |
| ATOM | 2510 | CB | SER | 312 | 29.040 | 43.653 | −4.645 | 1.00 | 13.83 |
| ATOM | 2511 | OG | SER | 312 | 28.821 | 43.933 | −3.284 | 1.00 | 20.95 |
| ATOM | 2512 | C | SER | 312 | 26.576 | 43.311 | −4.593 | 1.00 | 9.82 |
| ATOM | 2513 | O | SER | 312 | 26.000 | 44.059 | −3.793 | 1.00 | 11.18 |
| ATOM | 2514 | N | TYR | 313 | 26.260 | 42.025 | −4.815 | 1.00 | 9.06 |
| ATOM | 2515 | CA | TYR | 313 | 25.095 | 41.471 | −4.152 | 1.00 | 8.56 |
| ATOM | 2516 | CB | TYR | 313 | 24.722 | 40.082 | −4.734 | 1.00 | 9.03 |
| ATOM | 2517 | CG | TYR | 313 | 23.265 | 39.812 | −4.375 | 1.00 | 7.11 |
| ATOM | 2518 | CD1 | TYR | 313 | 22.290 | 40.192 | −5.276 | 1.00 | 8.14 |
| ATOM | 2519 | CE1 | TYR | 313 | 20.951 | 40.004 | −5.038 | 1.00 | 8.04 |
| ATOM | 2520 | CD2 | TYR | 313 | 22.871 | 39.273 | −3.179 | 1.00 | 6.33 |
| ATOM | 2521 | CE2 | TYR | 313 | 21.525 | 39.151 | −2.889 | 1.00 | 6.78 |
| ATOM | 2522 | CZ | TYR | 313 | 20.558 | 39.481 | −3.810 | 1.00 | 6.87 |
| ATOM | 2523 | OH | TYR | 313 | 19.227 | 39.413 | −3.570 | 1.00 | 7.92 |
| ATOM | 2524 | C | TYR | 313 | 25.247 | 41.465 | −2.642 | 1.00 | 8.88 |
| ATOM | 2525 | O | TYR | 313 | 24.295 | 41.721 | −1.876 | 1.00 | 8.66 |
| ATOM | 2526 | N | GLY | 314 | 26.436 | 41.154 | −2.137 | 1.00 | 9.27 |
| ATOM | 2527 | CA | GLY | 314 | 26.636 | 41.130 | −0.699 | 1.00 | 10.93 |
| ATOM | 2528 | C | GLY | 314 | 26.438 | 42.475 | −0.028 | 1.00 | 11.24 |
| ATOM | 2529 | O | GLY | 314 | 25.837 | 42.532 | 1.044 | 1.00 | 11.92 |
| ATOM | 2530 | N | ASP | 315 | 26.927 | 43.543 | −0.658 | 1.00 | 12.52 |
| ATOM | 2531 | CA | ASP | 315 | 26.682 | 44.876 | −0.145 | 1.00 | 13.51 |
| ATOM | 2532 | C | ASP | 315 | 25.178 | 45.169 | −0.126 | 1.00 | 11.36 |
| ATOM | 2533 | O | ASP | 315 | 24.643 | 45.674 | 0.871 | 1.00 | 12.03 |
| ATOM | 2534 | CB | ASP | 315 | 27.367 | 45.918 | −1.023 | 1.00 | 18.60 |
| ATOM | 2535 | CG | ASP | 315 | 28.845 | 46.015 | −0.671 | 1.00 | 23.51 |
| ATOM | 2536 | OD1 | ASP | 315 | 29.308 | 45.275 | 0.214 | 1.00 | 34.09 |
| ATOM | 2537 | OD2 | ASP | 315 | 29.448 | 46.876 | −1.358 | 1.00 | 38.29 |
| ATOM | 2538 | N | TYR | 316 | 24.538 | 44.902 | −1.265 | 1.00 | 10.83 |
| ATOM | 2539 | CA | TYR | 316 | 23.095 | 45.065 | −1.383 | 1.00 | 9.46 |
| ATOM | 2540 | CB | TYR | 316 | 22.621 | 44.578 | −2.740 | 1.00 | 9.26 |
| ATOM | 2541 | CG | TYR | 316 | 21.130 | 44.397 | −2.840 | 1.00 | 8.60 |
| ATOM | 2542 | CD1 | TYR | 316 | 20.273 | 45.477 | −3.037 | 1.00 | 8.34 |
| ATOM | 2543 | CE1 | TYR | 316 | 18.913 | 45.293 | −3.143 | 1.00 | 7.74 |
| ATOM | 2544 | CD2 | TYR | 316 | 20.585 | 43.119 | −2.768 | 1.00 | 7.91 |
| ATOM | 2545 | CE2 | TYR | 316 | 19.210 | 42.953 | −2.847 | 1.00 | 7.88 |
| ATOM | 2546 | CZ | TYR | 316 | 18.381 | 44.028 | −3.021 | 1.00 | 7.84 |
| ATOM | 2547 | OH | TYR | 316 | 17.021 | 43.799 | −3.101 | 1.00 | 8.21 |
| ATOM | 2548 | C | TYR | 316 | 22.328 | 44.386 | −0.260 | 1.00 | 8.23 |
| ATOM | 2549 | O | TYR | 316 | 21.435 | 44.940 | 0.393 | 1.00 | 8.68 |
| ATOM | 2550 | N | LEU | 317 | 22.637 | 43.104 | −0.045 | 1.00 | 8.47 |
| ATOM | 2551 | CA | LEU | 317 | 21.902 | 42.282 | 0.915 | 1.00 | 8.58 |
| ATOM | 2552 | CB | LEU | 317 | 22.285 | 40.787 | 0.743 | 1.00 | 8.79 |
| ATOM | 2553 | CG | LEU | 317 | 21.561 | 39.779 | 1.629 | 1.00 | 8.20 |
| ATOM | 2554 | CD1 | LEU | 317 | 20.073 | 39.788 | 1.272 | 1.00 | 9.81 |
| ATOM | 2555 | CD2 | LEU | 317 | 22.141 | 38.375 | 1.492 | 1.00 | 10.08 |
| ATOM | 2556 | C | LEU | 317 | 22.107 | 42.735 | 2.352 | 1.00 | 8.90 |
| ATOM | 2557 | O | LEU | 317 | 21.144 | 42.841 | 3.087 | 1.00 | 9.53 |
| ATOM | 2558 | N | GLN | 318 | 23.362 | 42.918 | 2.770 | 1.00 | 10.62 |
| ATOM | 2559 | CA | GLN | 318 | 23.615 | 43.262 | 4.185 | 1.00 | 12.66 |
| ATOM | 2560 | CB | GLN | 318 | 25.124 | 43.337 | 4.464 | 1.00 | 14.04 |
| ATOM | 2561 | CG | GLN | 318 | 25.445 | 43.620 | 5.905 | 1.00 | 19.74 |
| ATOM | 2562 | CD | GLN | 318 | 26.927 | 43.890 | 6.154 | 1.00 | 23.96 |
| ATOM | 2563 | OE1 | GLN | 318 | 27.714 | 44.211 | 5.249 | 1.00 | 35.18 |
| ATOM | 2564 | NE2 | GLN | 318 | 27.322 | 43.856 | 7.426 | 1.00 | 29.80 |
| ATOM | 2565 | C | GLN | 318 | 22.915 | 44.561 | 4.553 | 1.00 | 12.86 |
| ATOM | 2566 | O | GLN | 318 | 22.198 | 44.673 | 5.560 | 1.00 | 15.11 |
| ATOM | 2567 | N | ASN | 319 | 23.013 | 45.524 | 3.641 | 1.00 | 12.55 |
| ATOM | 2568 | CA | ASN | 319 | 22.346 | 46.806 | 3.917 | 1.00 | 14.03 |
| ATOM | 2569 | CB | ASN | 319 | 23.012 | 47.860 | 3.043 | 1.00 | 20.38 |
| ATOM | 2570 | CG | ASN | 319 | 24.469 | 48.100 | 3.472 | 1.00 | 27.04 |
| ATOM | 2571 | OD1 | ASN | 319 | 24.837 | 48.041 | 4.669 | 1.00 | 36.52 |
| ATOM | 2572 | ND2 | ASN | 319 | 25.310 | 48.297 | 2.465 | 1.00 | 38.86 |
| ATOM | 2573 | C | ASN | 319 | 20.844 | 46.703 | 3.764 | 1.00 | 11.80 |
| ATOM | 2574 | O | ASN | 319 | 20.143 | 47.296 | 4.593 | 1.00 | 11.96 |
| ATOM | 2575 | N | GLY | 320 | 20.312 | 45.946 | 2.806 | 1.00 | 9.58 |
| ATOM | 2576 | CA | GLY | 320 | 18.902 | 45.829 | 2.624 | 1.00 | 9.89 |
| ATOM | 2577 | C | GLY | 320 | 18.179 | 45.194 | 3.797 | 1.00 | 9.39 |
| ATOM | 2578 | O | GLY | 320 | 17.091 | 45.598 | 4.167 | 1.00 | 10.40 |
| ATOM | 2579 | N | LEU | 321 | 18.797 | 44.151 | 4.363 | 1.00 | 9.14 |
| ATOM | 2580 | CA | LEU | 321 | 18.153 | 43.464 | 5.485 | 1.00 | 9.96 |
| ATOM | 2581 | CB | LEU | 321 | 18.857 | 42.133 | 5.768 | 1.00 | 10.64 |
| ATOM | 2582 | CG | LEU | 321 | 18.723 | 41.078 | 4.638 | 1.00 | 10.47 |
| ATOM | 2583 | CD1 | LEU | 321 | 19.399 | 39.826 | 5.109 | 1.00 | 15.39 |
| ATOM | 2584 | CD2 | LEU | 321 | 17.262 | 40.824 | 4.233 | 1.00 | 12.12 |
| ATOM | 2585 | C | LEU | 321 | 18.108 | 44.339 | 6.710 | 1.00 | 10.07 |
| ATOM | 2586 | O | LEU | 321 | 17.089 | 44.340 | 7.437 | 1.00 | 10.21 |
| ATOM | 2587 | N | VAL | 322 | 19.176 | 45.095 | 6.956 | 1.00 | 10.17 |
| ATOM | 2588 | CA | VAL | 322 | 19.146 | 46.035 | 8.069 | 1.00 | 11.92 |
| ATOM | 2589 | CB | VAL | 322 | 20.532 | 46.687 | 8.252 | 1.00 | 14.76 |
| ATOM | 2590 | CG1 | VAL | 322 | 20.397 | 47.770 | 9.346 | 1.00 | 20.10 |
| ATOM | 2591 | CG2 | VAL | 322 | 21.537 | 45.651 | 8.753 | 1.00 | 21.00 |
| ATOM | 2592 | C | VAL | 322 | 18.095 | 47.100 | 7.854 | 1.00 | 10.68 |
| ATOM | 2593 | O | VAL | 322 | 17.346 | 47.521 | 8.741 | 1.00 | 12.21 |
| ATOM | 2594 | N | SER | 323 | 18.014 | 47.617 | 6.634 | 1.00 | 11.81 |
| ATOM | 2595 | CA | SER | 323 | 17.069 | 48.683 | 6.345 | 1.00 | 11.28 |
| ATOM | 2596 | CB | SER | 323 | 17.295 | 49.233 | 4.922 | 1.00 | 14.31 |
| ATOM | 2597 | OG | SER | 323 | 18.592 | 49.829 | 4.835 | 1.00 | 19.95 |
| ATOM | 2598 | C | SER | 323 | 15.625 | 48.220 | 6.522 | 1.00 | 11.18 |
| ATOM | 2599 | O | SER | 323 | 14.776 | 48.982 | 6.976 | 1.00 | 12.09 |
| ATOM | 2600 | N | LEU | 324 | 15.345 | 46.979 | 6.128 | 1.00 | 11.02 |
| ATOM | 2601 | CA | LEU | 324 | 13.986 | 46.458 | 6.212 | 1.00 | 9.92 |
| ATOM | 2602 | CB | LEU | 324 | 13.893 | 45.156 | 5.430 | 1.00 | 9.96 |
| ATOM | 2603 | CG | LEU | 324 | 12.522 | 44.506 | 5.348 | 1.00 | 9.98 |
| ATOM | 2604 | CD1 | LEU | 324 | 11.448 | 45.393 | 4.776 | 1.00 | 11.33 |
| ATOM | 2605 | CD2 | LEU | 324 | 12.658 | 43.214 | 4.571 | 1.00 | 11.39 |
| ATOM | 2606 | C | LEU | 324 | 13.576 | 46.326 | 7.679 | 1.00 | 10.28 |
| ATOM | 2607 | O | LEU | 324 | 12.422 | 46.540 | 8.070 | 1.00 | 10.32 |
| ATOM | 2608 | N | ILE | 325 | 14.532 | 45.886 | 8.497 | 1.00 | 10.22 |
| ATOM | 2609 | CA | ILE | 325 | 14.292 | 45.805 | 9.939 | 1.00 | 11.04 |
| ATOM | 2610 | CB | ILE | 325 | 15.451 | 45.120 | 10.669 | 1.00 | 10.87 |
| ATOM | 2611 | CG2 | ILE | 325 | 15.398 | 45.298 | 12.164 | 1.00 | 13.93 |
| ATOM | 2612 | CG1 | ILE | 325 | 15.454 | 43.605 | 10.363 | 1.00 | 12.46 |
| ATOM | 2613 | CD1 | ILE | 325 | 16.785 | 42.944 | 10.725 | 1.00 | 15.56 |
| ATOM | 2614 | C | ILE | 325 | 14.040 | 47.192 | 10.522 | 1.00 | 10.74 |
| ATOM | 2615 | O | ILE | 325 | 13.122 | 47.404 | 11.341 | 1.00 | 11.60 |
| ATOM | 2616 | N | ASN | 326 | 14.819 | 48.181 | 10.151 | 1.00 | 10.99 |
| ATOM | 2617 | CA | ASN | 326 | 14.602 | 49.531 | 10.659 | 1.00 | 12.82 |
| ATOM | 2618 | CB | ASN | 326 | 15.732 | 50.459 | 10.168 | 1.00 | 14.29 |
| ATOM | 2619 | CG | ASN | 326 | 17.014 | 50.101 | 10.904 | 1.00 | 17.21 |
| ATOM | 2620 | OD1 | ASN | 326 | 16.959 | 49.382 | 11.917 | 1.00 | 23.79 |
| ATOM | 2621 | ND2 | ASN | 326 | 18.130 | 50.520 | 10.318 | 1.00 | 22.06 |
| ATOM | 2622 | C | ASN | 326 | 13.265 | 50.066 | 10.203 | 1.00 | 12.41 |
| ATOM | 2623 | O | ASN | 326 | 12.620 | 50.749 | 10.996 | 1.00 | 14.07 |
| ATOM | 2624 | N | LYS | 327 | 12.840 | 49.790 | 8.973 | 1.00 | 11.71 |
| ATOM | 2625 | CA | LYS | 327 | 11.586 | 50.307 | 8.468 | 1.00 | 12.41 |
| ATOM | 2626 | CB | LYS | 327 | 11.543 | 50.205 | 6.918 | 1.00 | 13.45 |
| ATOM | 2627 | CG | LYS | 327 | 10.362 | 50.865 | 6.259 | 1.00 | 18.26 |
| ATOM | 2628 | CD | LYS | 327 | 9.847 | 50.144 | 5.014 | 1.00 | 21.08 |
| ATOM | 2629 | CE | LYS | 327 | 8.605 | 50.768 | 4.423 | 1.00 | 21.51 |
| ATOM | 2630 | NZ | LYS | 327 | 7.335 | 50.650 | 5.211 | 1.00 | 17.68 |
| ATOM | 2631 | C | LYS | 327 | 10.349 | 49.653 | 9.067 | 1.00 | 11.15 |
| ATOM | 2632 | O | LYS | 327 | 9.389 | 50.327 | 9.506 | 1.00 | 12.52 |
| ATOM | 2633 | N | ASN | 328 | 10.358 | 48.303 | 8.990 | 1.00 | 10.58 |
| ATOM | 2634 | CA | ASN | 328 | 9.160 | 47.510 | 9.286 | 1.00 | 11.33 |
| ATOM | 2635 | CB | ASN | 328 | 8.762 | 46.614 | 8.084 | 1.00 | 14.02 |
| ATOM | 2636 | CG | ASN | 328 | 8.096 | 47.438 | 7.002 | 1.00 | 14.11 |
| ATOM | 2637 | OD1 | ASN | 328 | 7.997 | 48.681 | 7.112 | 1.00 | 16.02 |
| ATOM | 2638 | ND2 | ASN | 328 | 7.525 | 46.796 | 5.995 | 1.00 | 11.03 |
| ATOM | 2639 | C | ASN | 328 | 9.224 | 46.671 | 10.551 | 1.00 | 11.26 |
| ATOM | 2640 | O | ASN | 328 | 8.226 | 45.994 | 10.873 | 1.00 | 13.57 |
| ATOM | 2641 | N | GLY | 329 | 10.341 | 46.732 | 11.270 | 1.00 | 12.34 |
| ATOM | 2642 | CA | GLY | 329 | 10.445 | 46.076 | 12.554 | 1.00 | 12.29 |
| ATOM | 2643 | C | GLY | 329 | 11.146 | 44.739 | 12.523 | 1.00 | 11.30 |
| ATOM | 2644 | O | GLY | 329 | 11.221 | 44.056 | 11.517 | 1.00 | 11.94 |
| ATOM | 2645 | N | GLN | 330 | 11.716 | 44.359 | 13.668 | 1.00 | 11.96 |
| ATOM | 2646 | CA | GLN | 330 | 12.364 | 43.036 | 13.787 | 1.00 | 10.55 |
| ATOM | 2647 | CB | GLN | 330 | 13.193 | 42.950 | 15.072 | 1.00 | 11.32 |
| ATOM | 2648 | CG | GLN | 330 | 13.686 | 41.560 | 15.352 | 1.00 | 11.79 |
| ATOM | 2649 | CD | GLN | 330 | 14.755 | 41.131 | 14.371 | 1.00 | 10.04 |
| ATOM | 2650 | OE1 | GLN | 330 | 15.804 | 41.744 | 14.168 | 1.00 | 13.20 |
| ATOM | 2651 | NE2 | GLN | 330 | 14.469 | 40.024 | 13.733 | 1.00 | 11.43 |
| ATOM | 2652 | C | GLN | 330 | 11.291 | 41.961 | 13.747 | 1.00 | 10.94 |
| ATOM | 2653 | O | GLN | 330 | 10.401 | 41.952 | 14.592 | 1.00 | 12.29 |
| ATOM | 2654 | N | THR | 331 | 11.377 | 41.063 | 12.784 | 1.00 | 9.93 |
| ATOM | 2655 | CA | THR | 331 | 10.438 | 39.956 | 12.692 | 1.00 | 10.56 |
| ATOM | 2656 | CB | THR | 331 | 10.367 | 39.375 | 11.277 | 1.00 | 13.12 |
| ATOM | 2657 | OG1 | THR | 331 | 11.627 | 38.829 | 10.872 | 1.00 | 14.79 |
| ATOM | 2658 | CG2 | THR | 331 | 9.945 | 40.455 | 10.274 | 1.00 | 18.32 |
| ATOM | 2659 | C | THR | 331 | 10.801 | 38.843 | 13.669 | 1.00 | 9.68 |
| ATOM | 2660 | O | THR | 331 | 9.996 | 37.874 | 13.694 | 1.00 | 10.45 |
| ATOM | 2661 | OT | THR | 331 | 11.803 | 38.971 | 14.419 | 1.00 | 9.25 |
| ATOM | 2662 | OW | WAT | 333 | 9.679 | 28.766 | −0.715 | 1.00 | 6.49 |
| ATOM | 2663 | OW | WAT | 334 | 19.171 | 27.783 | 10.936 | 1.00 | 7.24 |
| ATOM | 2664 | OW | WAT | 335 | 9.260 | 43.735 | −4.195 | 1.00 | 8.08 |
| ATOM | 2665 | OW | WAT | 336 | 22.532 | 31.208 | 2.029 | 1.00 | 6.26 |
| ATOM | 2666 | OW | WAT | 337 | 28.595 | 34.041 | 1.151 | 1.00 | 7.97 |
| ATOM | 2667 | OW | WAT | 338 | 24.607 | 26.075 | 6.861 | 1.00 | 8.47 |
| ATOM | 2668 | OW | WAT | 339 | −2.784 | 36.461 | −0.561 | 1.00 | 8.55 |
| ATOM | 2669 | OW | WAT | 340 | 22.156 | 23.031 | 7.059 | 1.00 | 7.69 |
| ATOM | 2670 | OW | WAT | 341 | 14.777 | 39.110 | 1.828 | 1.00 | 8.58 |
| ATOM | 2671 | OW | WAT | 342 | 12.607 | 41.059 | 1.589 | 1.00 | 9.25 |
| ATOM | 2672 | OW | WAT | 343 | −1.547 | 35.753 | 12.742 | 1.00 | 9.09 |
| ATOM | 2673 | OW | WAT | 344 | 15.859 | 16.926 | 12.366 | 1.00 | 8.72 |
| ATOM | 2674 | OW | WAT | 345 | 17.270 | 37.486 | 2.078 | 1.00 | 8.11 |
| ATOM | 2675 | OW | WAT | 346 | 3.657 | 33.965 | −24.602 | 1.00 | 10.08 |
| ATOM | 2676 | OW | WAT | 347 | 25.532 | 21.232 | −3.227 | 1.00 | 9.87 |
| ATOM | 2677 | OW | WAT | 348 | −2.697 | 35.006 | 10.127 | 1.00 | 9.24 |
| ATOM | 2678 | OW | WAT | 349 | −1.983 | 38.149 | 8.911 | 1.00 | 9.83 |
| ATOM | 2679 | OW | WAT | 350 | 2.708 | 25.709 | 10.377 | 1.00 | 10.06 |
| ATOM | 2680 | OW | WAT | 351 | 1.466 | 29.802 | −2.581 | 1.00 | 10.39 |
| ATOM | 2681 | OW | WAT | 352 | 1.694 | 36.486 | 5.140 | 1.00 | 10.21 |
| ATOM | 2682 | OW | WAT | 353 | 14.787 | 40.054 | −27.891 | 1.00 | 11.32 |
| ATOM | 2683 | OW | WAT | 354 | 7.944 | 41.566 | −10.080 | 1.00 | 11.49 |
| ATOM | 2684 | OW | WAT | 355 | 10.898 | 19.364 | −0.933 | 1.00 | 11.45 |
| ATOM | 2685 | OW | WAT | 356 | 11.246 | 44.075 | 8.852 | 1.00 | 10.27 |
| ATOM | 2686 | OW | WAT | 357 | 19.835 | 30.921 | −23.876 | 1.00 | 12.14 |
| ATOM | 2687 | OW | WAT | 358 | 27.717 | 21.786 | −4.790 | 1.00 | 10.92 |
| ATOM | 2688 | OW | WAT | 359 | 25.375 | 32.089 | −5.869 | 1.00 | 11.81 |
| ATOM | 2689 | OW | WAT | 360 | 7.719 | 32.045 | 19.976 | 1.00 | 10.99 |
| ATOM | 2690 | OW | WAT | 361 | 12.903 | 41.900 | 8.252 | 1.00 | 10.19 |
| ATOM | 2691 | OW | WAT | 362 | 15.465 | 41.811 | 7.221 | 1.00 | 10.84 |
| ATOM | 2692 | OW | WAT | 363 | 12.347 | 17.857 | 1.029 | 1.00 | 12.37 |
| ATOM | 2693 | OW | WAT | 364 | 16.302 | 36.649 | −24.739 | 1.00 | 10.86 |
| ATOM | 2694 | OW | WAT | 365 | 10.865 | 30.651 | 24.027 | 1.00 | 12.28 |
| ATOM | 2695 | OW | WAT | 366 | 19.416 | 33.418 | −15.771 | 1.00 | 10.14 |
| ATOM | 2696 | OW | WAT | 367 | 0.655 | 27.761 | 10.957 | 1.00 | 11.04 |
| ATOM | 2697 | OW | WAT | 368 | 6.259 | 36.234 | −3.794 | 1.00 | 11.14 |
| ATOM | 2698 | OW | WAT | 369 | 16.675 | 14.973 | 9.695 | 1.00 | 11.88 |
| ATOM | 2699 | OW | WAT | 370 | 7.905 | 39.248 | −11.909 | 1.00 | 10.25 |
| ATOM | 2700 | OW | WAT | 371 | 18.361 | 15.936 | 2.863 | 1.00 | 11.46 |
| ATOM | 2701 | OW | WAT | 372 | 21.892 | 19.503 | 1.831 | 1.00 | 13.37 |
| ATOM | 2702 | OW | WAT | 373 | 7.417 | 33.809 | 23.230 | 1.00 | 11.13 |
| ATOM | 2703 | OW | WAT | 374 | 9.301 | 18.255 | −4.039 | 1.00 | 13.47 |
| ATOM | 2704 | OW | WAT | 375 | 5.788 | 38.293 | −2.097 | 1.00 | 12.75 |
| ATOM | 2705 | OW | WAT | 376 | 21.318 | 28.084 | 18.230 | 1.00 | 13.18 |
| ATOM | 2706 | OW | WAT | 377 | 5.087 | 41.215 | −22.821 | 1.00 | 13.37 |
| ATOM | 2707 | OW | WAT | 378 | 24.969 | 23.273 | 6.991 | 1.00 | 13.42 |
| ATOM | 2708 | OW | WAT | 379 | 11.227 | 49.320 | −8.022 | 1.00 | 13.43 |
| ATOM | 2709 | OW | WAT | 380 | −7.291 | 29.246 | 11.626 | 1.00 | 13.60 |
| ATOM | 2710 | OW | WAT | 381 | 5.640 | 46.235 | 9.550 | 1.00 | 13.85 |
| ATOM | 2711 | OW | WAT | 382 | 8.978 | 35.948 | 12.107 | 1.00 | 11.77 |
| ATOM | 2712 | OW | WAT | 383 | 6.906 | 29.370 | 22.628 | 1.00 | 15.24 |
| ATOM | 2713 | OW | WAT | 384 | 9.627 | 19.425 | 20.724 | 1.00 | 14.52 |
| ATOM | 2714 | OW | WAT | 385 | 16.459 | 39.497 | 8.314 | 1.00 | 14.01 |
| ATOM | 2715 | OW | WAT | 386 | 24.545 | 45.847 | −10.066 | 1.00 | 13.62 |
| ATOM | 2716 | OW | WAT | 387 | −0.150 | 40.151 | 13.142 | 1.00 | 15.04 |
| ATOM | 2717 | OW | WAT | 388 | 17.528 | 29.411 | −23.847 | 1.00 | 12.67 |
| ATOM | 2718 | OW | WAT | 389 | 11.478 | 50.549 | −25.585 | 1.00 | 14.89 |
| ATOM | 2719 | OW | WAT | 390 | 13.559 | 40.717 | 10.705 | 1.00 | 15.14 |
| ATOM | 2720 | OW | WAT | 391 | 8.290 | 18.854 | −0.320 | 1.00 | 13.16 |
| ATOM | 2721 | OW | WAT | 392 | 18.743 | 43.137 | −23.378 | 1.00 | 13.36 |
| ATOM | 2722 | OW | WAT | 393 | −0.660 | 20.811 | −4.126 | 1.00 | 14.45 |
| ATOM | 2723 | OW | WAT | 394 | 11.073 | 48.625 | 1.433 | 1.00 | 13.51 |
| ATOM | 2724 | OW | WAT | 395 | 21.541 | 28.028 | −11.200 | 1.00 | 16.03 |
| ATOM | 2725 | OW | WAT | 396 | −9.012 | 33.285 | 2.180 | 1.00 | 13.36 |
| ATOM | 2726 | OW | WAT | 397 | −5.015 | 37.842 | −7.595 | 1.00 | 14.72 |
| ATOM | 2727 | OW | WAT | 398 | 7.685 | 39.106 | −0.476 | 1.00 | 13.04 |
| ATOM | 2728 | OW | WAT | 399 | −2.609 | 52.730 | 2.926 | 1.00 | 15.18 |
| ATOM | 2729 | OW | WAT | 400 | 31.148 | 33.765 | −2.024 | 1.00 | 15.90 |
| ATOM | 2730 | OW | WAT | 401 | 28.412 | 25.681 | −6.948 | 1.00 | 14.37 |
| ATOM | 2731 | OW | WAT | 402 | −7.837 | 33.960 | −2.251 | 1.00 | 16.42 |
| ATOM | 2732 | OW | WAT | 403 | 27.733 | 30.817 | 11.858 | 1.00 | 16.15 |
| ATOM | 2733 | OW | WAT | 404 | 20.345 | 47.455 | −0.111 | 1.00 | 15.84 |
| ATOM | 2734 | OW | WAT | 405 | 7.740 | 46.885 | −13.836 | 1.00 | 15.47 |
| ATOM | 2735 | OW | WAT | 406 | −6.948 | 43.028 | 7.219 | 1.00 | 13.61 |
| ATOM | 2736 | OW | WAT | 407 | −1.255 | 31.160 | −1.492 | 1.00 | 15.03 |
| ATOM | 2737 | OW | WAT | 408 | −7.351 | 47.298 | 1.758 | 1.00 | 16.16 |
| ATOM | 2738 | OW | WAT | 409 | 0.600 | 50.511 | 3.412 | 1.00 | 16.57 |
| ATOM | 2739 | OW | WAT | 410 | 19.491 | 38.870 | 14.832 | 1.00 | 13.70 |
| ATOM | 2740 | OW | WAT | 411 | 19.032 | 29.394 | 25.238 | 1.00 | 13.82 |
| ATOM | 2741 | OW | WAT | 412 | 1.566 | 19.249 | −3.495 | 1.00 | 12.61 |
| ATOM | 2742 | OW | WAT | 413 | 1.396 | 29.458 | −19.005 | 1.00 | 17.83 |
| ATOM | 2743 | OW | WAT | 414 | 12.993 | 13.760 | 6.156 | 1.00 | 16.00 |
| ATOM | 2744 | OW | WAT | 415 | −3.489 | 25.740 | 2.588 | 1.00 | 14.57 |
| ATOM | 2745 | OW | WAT | 416 | 20.400 | 16.258 | 4.749 | 1.00 | 15.12 |
| ATOM | 2746 | OW | WAT | 417 | 8.420 | 43.590 | −11.863 | 1.00 | 15.17 |
| ATOM | 2747 | OW | WAT | 418 | 23.155 | 21.243 | −4.704 | 1.00 | 15.01 |
| ATOM | 2748 | OW | WAT | 419 | 13.407 | 49.512 | −6.246 | 1.00 | 18.08 |
| ATOM | 2749 | OW | WAT | 420 | 2.293 | 43.872 | −19.188 | 1.00 | 15.75 |
| ATOM | 2750 | OW | WAT | 421 | 16.464 | 23.984 | −12.729 | 1.00 | 16.55 |
| ATOM | 2751 | OW | WAT | 422 | 18.051 | 18.401 | 13.304 | 1.00 | 16.18 |
| ATOM | 2752 | OW | WAT | 423 | 2.749 | 32.610 | 17.294 | 1.00 | 16.03 |
| ATOM | 2753 | OW | WAT | 424 | 3.167 | 43.048 | −21.870 | 1.00 | 16.60 |
| ATOM | 2754 | OW | WAT | 425 | 1.729 | 36.092 | 20.156 | 1.00 | 16.00 |
| ATOM | 2755 | OW | WAT | 426 | 24.912 | 30.437 | 18.039 | 1.00 | 18.10 |
| ATOM | 2756 | OW | WAT | 427 | 1.661 | 37.179 | −17.778 | 1.00 | 16.68 |
| ATOM | 2757 | OW | WAT | 428 | 8.377 | 48.751 | −17.456 | 1.00 | 17.96 |
| ATOM | 2758 | OW | WAT | 429 | 4.193 | 48.686 | −6.577 | 1.00 | 16.18 |
| ATOM | 2759 | OW | WAT | 430 | 32.183 | 20.100 | 4.650 | 1.00 | 17.47 |
| ATOM | 2760 | OW | WAT | 431 | 10.701 | 20.889 | −9.309 | 1.00 | 17.06 |
| ATOM | 2761 | OW | WAT | 432 | 1.230 | 36.624 | −21.785 | 1.00 | 16.21 |
| ATOM | 2762 | OW | WAT | 433 | 23.224 | 53.219 | −9.124 | 1.00 | 16.77 |
| ATOM | 2763 | OW | WAT | 434 | 7.454 | 14.204 | −2.641 | 1.00 | 19.16 |
| ATOM | 2764 | OW | WAT | 435 | −3.493 | 18.204 | −1.008 | 1.00 | 16.26 |
| ATOM | 2765 | OW | WAT | 436 | 28.871 | 35.527 | −9.186 | 1.00 | 16.44 |
| ATOM | 2766 | OW | WAT | 437 | 28.827 | 47.359 | −4.440 | 1.00 | 20.15 |
| ATOM | 2767 | OW | WAT | 438 | 16.179 | 24.748 | −15.541 | 1.00 | 18.41 |
| ATOM | 2768 | OW | WAT | 439 | 24.130 | 23.189 | 10.125 | 1.00 | 15.71 |
| ATOM | 2769 | OW | WAT | 440 | 9.413 | 18.353 | 13.315 | 1.00 | 22.18 |
| ATOM | 2770 | OW | WAT | 441 | 8.848 | 18.233 | 10.527 | 1.00 | 19.65 |
| ATOM | 2771 | OW | WAT | 442 | 26.464 | 32.534 | 18.217 | 1.00 | 16.18 |
| ATOM | 2772 | OW | WAT | 443 | −7.877 | 38.342 | 4.061 | 1.00 | 17.91 |
| ATOM | 2773 | OW | WAT | 444 | 12.963 | 34.080 | 10.130 | 1.00 | 14.21 |
| ATOM | 2774 | OW | WAT | 445 | 5.117 | 27.600 | 16.871 | 1.00 | 17.53 |
| ATOM | 2775 | OW | WAT | 446 | −9.839 | 37.847 | 2.096 | 1.00 | 20.90 |
| ATOM | 2776 | OW | WAT | 447 | −1.745 | 32.409 | 3.793 | 1.00 | 20.56 |
| ATOM | 2777 | OW | WAT | 448 | 8.416 | 36.915 | 9.538 | 1.00 | 19.37 |
| ATOM | 2778 | OW | WAT | 449 | 13.442 | 46.906 | 0.805 | 1.00 | 15.63 |
| ATOM | 2779 | OW | WAT | 450 | 4.457 | 30.452 | 20.352 | 1.00 | 16.16 |
| ATOM | 2780 | OW | WAT | 451 | 8.792 | 16.265 | −0.627 | 1.00 | 17.14 |
| ATOM | 2781 | OW | WAT | 452 | −0.356 | 37.156 | 21.516 | 1.00 | 17.26 |
| ATOM | 2782 | OW | WAT | 453 | 11.477 | 23.152 | −22.757 | 1.00 | 21.00 |
| ATOM | 2783 | OW | WAT | 454 | 21.490 | 29.901 | 24.676 | 1.00 | 18.12 |
| ATOM | 2784 | OW | WAT | 455 | −9.438 | 38.109 | 10.367 | 1.00 | 20.23 |
| ATOM | 2785 | OW | WAT | 456 | 0.801 | 21.803 | −6.497 | 1.00 | 16.76 |
| ATOM | 2786 | OW | WAT | 457 | 19.962 | 49.749 | −14.695 | 1.00 | 19.53 |
| ATOM | 2787 | OW | WAT | 458 | 15.665 | 20.950 | 19.711 | 1.00 | 20.63 |
| ATOM | 2788 | OW | WAT | 459 | 22.253 | 42.588 | 7.507 | 1.00 | 18.83 |
| ATOM | 2789 | OW | WAT | 460 | 1.091 | 15.140 | −0.991 | 1.00 | 17.62 |
| ATOM | 2790 | OW | WAT | 461 | 15.096 | 47.428 | −1.171 | 1.00 | 18.28 |
| ATOM | 2791 | OW | WAT | 462 | 9.229 | 16.847 | 19.798 | 1.00 | 19.90 |
| ATOM | 2792 | OW | WAT | 463 | 23.458 | 31.087 | 12.465 | 1.00 | 20.21 |
| ATOM | 2793 | OW | WAT | 464 | 19.997 | 42.399 | 9.231 | 1.00 | 20.50 |
| ATOM | 2794 | OW | WAT | 465 | −1.338 | 22.340 | −1.994 | 1.00 | 18.86 |
| ATOM | 2795 | OW | WAT | 466 | 3.252 | 20.298 | −7.395 | 1.00 | 20.44 |
| ATOM | 2796 | OW | WAT | 467 | 13.042 | 53.167 | −27.095 | 1.00 | 19.91 |
| ATOM | 2797 | OW | WAT | 468 | −10.643 | 37.955 | 15.133 | 1.00 | 20.65 |
| ATOM | 2798 | OW | WAT | 469 | 13.185 | 21.680 | −8.488 | 1.00 | 20.52 |
| ATOM | 2799 | OW | WAT | 470 | 10.293 | 15.611 | 9.484 | 1.00 | 17.30 |
| ATOM | 2800 | OW | WAT | 471 | 18.301 | 39.511 | −27.728 | 1.00 | 15.98 |
| ATOM | 2801 | OW | WAT | 472 | 30.497 | 24.989 | −0.891 | 1.00 | 18.92 |
| ATOM | 2802 | OW | WAT | 473 | 34.106 | 27.545 | 11.353 | 1.00 | 20.62 |
| ATOM | 2803 | OW | WAT | 474 | −1.263 | 34.235 | −1.003 | 1.00 | 21.04 |
| ATOM | 2804 | OW | WAT | 475 | 30.740 | 34.281 | 8.033 | 1.00 | 22.42 |
| ATOM | 2805 | OW | WAT | 476 | 17.888 | 47.600 | −24.851 | 1.00 | 19.76 |
| ATOM | 2806 | OW | WAT | 477 | 19.023 | 45.815 | −22.920 | 1.00 | 19.37 |
| ATOM | 2807 | OW | WAT | 478 | 5.376 | 27.996 | −23.488 | 1.00 | 23.73 |
| ATOM | 2808 | OW | WAT | 479 | 18.268 | 40.811 | 13.239 | 1.00 | 19.22 |
| ATOM | 2809 | OW | WAT | 480 | −4.271 | 44.290 | −11.498 | 1.00 | 18.91 |
| ATOM | 2810 | OW | WAT | 481 | −10.443 | 35.240 | 1.254 | 1.00 | 19.73 |
| ATOM | 2811 | OW | WAT | 482 | 2.681 | 33.500 | 20.144 | 1.00 | 19.18 |
| ATOM | 2812 | OW | WAT | 483 | 19.770 | 15.947 | 12.144 | 1.00 | 20.32 |
| ATOM | 2813 | OW | WAT | 484 | 4.713 | 13.467 | 7.499 | 1.00 | 21.46 |
| ATOM | 2814 | OW | WAT | 485 | -8.355 | 31.805 | −0.398 | 1.00 | 23.32 |
| ATOM | 2815 | OW | WAT | 486 | 15.331 | 47.230 | 2.640 | 1.00 | 18.04 |
| ATOM | 2816 | OW | WAT | 487 | 25.206 | 36.975 | 8.919 | 1.00 | 23.97 |
| ATOM | 2817 | OW | WAT | 488 | 2.787 | 39.754 | 14.409 | 1.00 | 18.16 |
| ATOM | 2818 | OW | WAT | 489 | 2.364 | 46.924 | −9.024 | 1.00 | 21.20 |
| ATOM | 2819 | OW | WAT | 490 | 18.912 | 42.320 | −26.268 | 1.00 | 19.59 |
| ATOM | 2820 | OW | WAT | 491 | 9.332 | 14.150 | −7.989 | 1.00 | 22.28 |
| ATOM | 2821 | OW | WAT | 492 | 3.716 | 51.522 | −5.917 | 1.00 | 20.14 |
| ATOM | 2822 | OW | WAT | 493 | 30.485 | 19.369 | 6.691 | 1.00 | 22.71 |
| ATOM | 2823 | OW | WAT | 494 | −8.748 | 45.801 | 7.529 | 1.00 | 21.63 |
| ATOM | 2824 | OW | WAT | 495 | 11.868 | 16.205 | −2.683 | 1.00 | 21.66 |
| ATOM | 2825 | OW | WAT | 496 | 13.346 | 35.997 | 8.497 | 1.00 | 19.24 |
| ATOM | 2826 | OW | WAT | 497 | 0.972 | 40.899 | −13.028 | 1.00 | 21.95 |
| ATOM | 2827 | OW | WAT | 498 | 4.183 | 53.535 | −1.459 | 1.00 | 28.32 |
| ATOM | 2828 | OW | WAT | 499 | 30.346 | 39.016 | −10.546 | 1.00 | 20.11 |
| ATOM | 2829 | OW | WAT | 500 | 16.129 | 24.513 | −19.240 | 1.00 | 25.21 |
| ATOM | 2830 | OW | WAT | 501 | 10.923 | 41.632 | 17.779 | 1.00 | 28.12 |
| ATOM | 2831 | OW | WAT | 502 | 18.809 | 24.865 | −19.164 | 1.00 | 22.89 |
| ATOM | 2832 | OW | WAT | 503 | 16.648 | 14.113 | 0.751 | 1.00 | 19.20 |
| ATOM | 2833 | OW | WAT | 504 | 19.213 | 39.701 | 8.979 | 1.00 | 20.79 |
| ATOM | 2834 | OW | WAT | 505 | 24.711 | 56.540 | −10.148 | 1.00 | 20.81 |
| ATOM | 2835 | OW | WAT | 506 | 22.101 | 29.548 | −23.677 | 1.00 | 22.77 |
| ATOM | 2836 | OW | WAT | 507 | 21.631 | 41.072 | 20.961 | 1.00 | 24.26 |
| ATOM | 2837 | OW | WAT | 508 | −3.925 | 32.996 | −15.355 | 1.00 | 25.98 |
| ATOM | 2838 | OW | WAT | 509 | −3.683 | 27.982 | 6.567 | 1.00 | 23.91 |
| ATOM | 2839 | OW | WAT | 510 | 22.548 | 22.934 | 15.189 | 1.00 | 24.78 |
| ATOM | 2840 | OW | WAT | 511 | 3.233 | 21.643 | −9.764 | 1.00 | 21.05 |
| ATOM | 2841 | OW | WAT | 512 | 33.443 | 23.225 | 2.328 | 1.00 | 24.54 |
| ATOM | 2842 | OW | WAT | 513 | 24.602 | 43.728 | −18.078 | 1.00 | 25.43 |
| ATOM | 2843 | OW | WAT | 514 | 16.686 | 43.816 | 15.797 | 1.00 | 22.61 |
| ATOM | 2844 | OW | WAT | 515 | 10.964 | 18.976 | −6.714 | 1.00 | 26.05 |
| ATOM | 2845 | OW | WAT | 516 | 0.840 | 16.582 | −3.289 | 1.00 | 22.32 |
| ATOM | 2846 | OW | WAT | 517 | −3.923 | 22.464 | −1.744 | 1.00 | 29.01 |
| ATOM | 2847 | OW | WAT | 518 | −0.997 | 25.906 | 9.408 | 1.00 | 27.67 |
| ATOM | 2848 | OW | WAT | 519 | 3.066 | 45.067 | −23.581 | 1.00 | 21.23 |
| ATOM | 2849 | OW | WAT | 520 | 20.631 | 16.301 | 16.915 | 1.00 | 29.84 |
| ATOM | 2850 | OW | WAT | 521 | 3.683 | 28.042 | −20.317 | 1.00 | 29.99 |
| ATOM | 2851 | OW | WAT | 522 | −7.926 | 27.757 | 3.844 | 1.00 | 23.89 |
| ATOM | 2852 | OW | WAT | 523 | 1.150 | 23.857 | −9.846 | 1.00 | 21.74 |
| ATOM | 2853 | OW | WAT | 524 | 13.889 | 16.199 | −5.074 | 1.00 | 22.55 |
| ATOM | 2854 | OW | WAT | 525 | −1.704 | 53.692 | −2.952 | 1.00 | 32.09 |
| ATOM | 2855 | OW | WAT | 526 | 30.576 | 35.496 | −4.718 | 1.00 | 26.56 |
| ATOM | 2856 | OW | WAT | 527 | 7.959 | 27.774 | −32.333 | 1.00 | 22.85 |
| ATOM | 2857 | OW | WAT | 528 | 0.310 | 39.649 | −17.650 | 1.00 | 26.17 |
| ATOM | 2858 | OW | WAT | 529 | −0.573 | 40.681 | −15.285 | 1.00 | 24.02 |
| ATOM | 2859 | OW | WAT | 530 | −5.413 | 37.314 | −11.579 | 1.00 | 26.13 |
| ATOM | 2860 | OW | WAT | 531 | 20.453 | 25.296 | −22.221 | 1.00 | 26.01 |
| ATOM | 2861 | OW | WAT | 532 | 2.287 | 15.472 | −5.046 | 1.00 | 27.90 |
| ATOM | 2862 | OW | WAT | 533 | 30.000 | 42.526 | −1.756 | 1.00 | 25.99 |
| ATOM | 2863 | OW | WAT | 534 | 13.014 | 48.338 | 13.867 | 1.00 | 29.93 |
| ATOM | 2864 | OW | WAT | 535 | 19.089 | 59.470 | −16.490 | 1.00 | 25.60 |
| ATOM | 2865 | OW | WAT | 536 | 23.246 | 37.608 | −22.518 | 1.00 | 30.37 |
| ATOM | 2866 | OW | WAT | 537 | 18.012 | 23.775 | −22.832 | 1.00 | 35.14 |
| ATOM | 2867 | OW | WAT | 538 | 32.942 | 31.103 | −1.587 | 1.00 | 27.55 |
| ATOM | 2868 | OW | WAT | 539 | 24.244 | 39.395 | 8.376 | 1.00 | 26.84 |
| ATOM | 2869 | OW | WAT | 540 | 16.151 | 39.516 | 11.126 | 1.00 | 27.39 |
| ATOM | 2870 | OW | WAT | 541 | −9.496 | 38.640 | 6.232 | 1.00 | 23.00 |
| ATOM | 2871 | OW | WAT | 542 | 11.570 | 53.681 | −24.197 | 1.00 | 25.04 |
| ATOM | 2872 | OW | WAT | 543 | 5.652 | 39.623 | 9.901 | 1.00 | 24.86 |
| ATOM | 2873 | OW | WAT | 544 | 15.243 | 51.336 | −7.590 | 1.00 | 31.58 |
| ATOM | 2874 | OW | WAT | 545 | 21.732 | 45.731 | −22.796 | 1.00 | 25.40 |
| ATOM | 2875 | OW | WAT | 546 | 26.109 | 29.562 | 15.747 | 1.00 | 26.48 |
| ATOM | 2876 | OW | WAT | 547 | 5.300 | 48.774 | 10.712 | 1.00 | 22.97 |
| ATOM | 2877 | OW | WAT | 548 | 16.333 | 19.082 | −6.041 | 1.00 | 31.87 |
| ATOM | 2878 | OW | WAT | 549 | 34.477 | 39.693 | −0.433 | 1.00 | 24.27 |
| ATOM | 2879 | OW | WAT | 550 | 32.307 | 28.802 | −2.454 | 1.00 | 28.07 |
| ATOM | 2880 | OW | WAT | 551 | 16.750 | 23.348 | 20.119 | 1.00 | 30.93 |
| ATOM | 2881 | OW | WAT | 552 | 19.254 | 45.692 | 25.110 | 1.00 | 30.02 |
| ATOM | 2882 | OW | WAT | 553 | 7.615 | 43.287 | 12.031 | 1.00 | 31.35 |
| ATOM | 2883 | OW | WAT | 554 | 21.139 | 41.273 | 15.275 | 1.00 | 24.54 |
| ATOM | 2884 | OW | WAT | 555 | −9.531 | 43.159 | 1.000 | 1.00 | 28.18 |
| ATOM | 2885 | OW | WAT | 556 | −4.562 | 35.560 | 22.961 | 1.00 | 25.96 |
| ATOM | 2886 | OW | WAT | 557 | 19.748 | 24.192 | −10.428 | 1.00 | 29.93 |
| ATOM | 2887 | OW | WAT | 558 | 10.358 | 13.845 | 7.421 | 1.00 | 24.97 |
| ATOM | 2888 | OW | WAT | 559 | 33.144 | 26.300 | −1.473 | 1.00 | 23.51 |
| ATOM | 2889 | OW | WAT | 560 | 0.711 | 42.085 | −22.328 | 1.00 | 27.47 |
| ATOM | 2890 | OW | WAT | 561 | 19.258 | 55.289 | −14.564 | 1.00 | 25.35 |
| ATOM | 2891 | OW | WAT | 562 | 13.683 | 49.398 | −2.033 | 1.00 | 27.95 |
| ATOM | 2892 | OW | WAT | 563 | 21.974 | 39.944 | 7.537 | 1.00 | 24.21 |
| ATOM | 2893 | OW | WAT | 564 | 14.094 | 24.261 | −29.685 | 1.00 | 30.81 |
| ATOM | 2894 | OW | WAT | 565 | 8.391 | 16.742 | 16.583 | 1.00 | 33.63 |
| ATOM | 2895 | OW | WAT | 566 | 34.902 | 40.206 | 3.922 | 1.00 | 34.09 |
| ATOM | 2896 | OW | WAT | 567 | 7.246 | 39.309 | 7.727 | 1.00 | 25.05 |
| ATOM | 2897 | OW | WAT | 568 | 1.772 | 52.043 | −7.936 | 1.00 | 33.11 |
| ATOM | 2898 | OW | WAT | 569 | −10.176 | 35.406 | −1.420 | 1.00 | 28.71 |
| ATOM | 2899 | OW | WAT | 570 | 19.034 | 21.727 | −6.972 | 1.00 | 31.14 |
| ATOM | 2900 | OW | WAT | 571 | 25.186 | 25.032 | 13.807 | 1.00 | 29.45 |
| ATOM | 2901 | OW | WAT | 572 | −0.477 | 22.506 | 0.681 | 1.00 | 29.75 |
| ATOM | 2902 | OW | WAT | 573 | 7.554 | 13.615 | 9.613 | 1.00 | 25.51 |
| ATOM | 2903 | OW | WAT | 574 | 0.741 | 15.993 | −6.797 | 1.00 | 28.22 |
| ATOM | 2904 | OW | WAT | 575 | 4.524 | 26.932 | 19.683 | 1.00 | 30.25 |
| ATOM | 2905 | OW | WAT | 576 | 24.217 | 31.560 | 29.964 | 1.00 | 30.98 |
| ATOM | 2906 | OW | WAT | 577 | −9.886 | 38.987 | −0.391 | 1.00 | 30.58 |
| ATOM | 2907 | OW | WAT | 578 | 18.264 | 48.710 | −5.256 | 1.00 | 26.25 |
| ATOM | 2908 | OW | WAT | 579 | 7.094 | 48.558 | −19.857 | 1.00 | 30.55 |
| ATOM | 2909 | OW | WAT | 580 | −11.403 | 38.772 | 12.578 | 1.00 | 23.49 |
| ATOM | 2910 | OW | WAT | 581 | 0.236 | 53.067 | 3.666 | 1.00 | 32.85 |
| ATOM | 2911 | OW | WAT | 582 | 34.494 | 30.211 | 11.953 | 1.00 | 30.38 |
| ATOM | 2912 | OW | WAT | 583 | −8.883 | 40.085 | 8.563 | 1.00 | 20.61 |
| ATOM | 2913 | OW | WAT | 584 | 19.648 | 16.274 | −1.256 | 1.00 | 32.05 |
| ATOM | 2914 | OW | WAT | 585 | 0.789 | 53.240 | 6.163 | 1.00 | 28.48 |
| ATOM | 2915 | OW | WAT | 586 | 6.772 | 16.061 | 19.133 | 1.00 | 28.67 |
| ATOM | 2916 | OW | WAT | 587 | 17.572 | 48.350 | −0.455 | 1.00 | 31.70 |
| ATOM | 2917 | OW | WAT | 588 | 19.914 | 42.743 | 12.082 | 1.00 | 29.47 |
| ATOM | 2918 | OW | WAT | 589 | 28.293 | 43.369 | 3.095 | 1.00 | 41.10 |
| ATOM | 2919 | OW | WAT | 590 | 4.140 | 16.905 | −6.015 | 1.00 | 33.04 |
| ATOM | 2920 | OW | WAT | 591 | −7.536 | 49.473 | 0.700 | 1.00 | 26.21 |
| ATOM | 2921 | OW | WAT | 592 | 16.545 | 11.527 | 7.703 | 1.00 | 35.77 |
| ATOM | 2922 | OW | WAT | 593 | 21.751 | 26.587 | −18.455 | 1.00 | 36.98 |
| ATOM | 2923 | OW | WAT | 594 | 28.027 | 36.486 | 9.408 | 1.00 | 30.24 |
| ATOM | 2924 | OW | WAT | 595 | −3.668 | 27.781 | 16.465 | 1.00 | 32.59 |
| ATOM | 2925 | OW | WAT | 596 | 6.641 | 50.716 | 9.132 | 1.00 | 30.57 |
| ATOM | 2926 | OW | WAT | 597 | 14.904 | 54.419 | −12.497 | 1.00 | 35.34 |
| ATOM | 2927 | OW | WAT | 598 | 13.687 | 41.518 | 18.737 | 1.00 | 28.20 |
| ATOM | 2928 | OW | WAT | 599 | 15.809 | 10.449 | 13.628 | 1.00 | 27.51 |
| ATOM | 2929 | OW | WAT | 600 | 0.266 | 35.585 | −19.094 | 1.00 | 32.22 |
| ATOM | 2930 | OW | WAT | 601 | 1.157 | 32.250 | −2.186 | 1.00 | 31.93 |
| ATOM | 2931 | OW | WAT | 602 | 20.830 | 54.594 | −22.978 | 1.00 | 38.78 |
| ATOM | 2932 | OW | WAT | 603 | −6.482 | 24.335 | 0.209 | 1.00 | 27.40 |
| ATOM | 2933 | OW | WAT | 604 | −0.221 | 24.757 | −19.652 | 1.00 | 34.87 |
| ATOM | 2934 | OW | WAT | 605 | 4.475 | 41.359 | 13.507 | 1.00 | 38.95 |
| ATOM | 2935 | OW | WAT | 606 | 18.365 | 17.118 | −5.002 | 1.00 | 35.63 |
| ATOM | 2936 | OW | WAT | 607 | 10.129 | 37.103 | 7.607 | 1.00 | 37.59 |
| ATOM | 2937 | OW | WAT | 608 | 32.483 | 26.313 | −6.257 | 1.00 | 34.83 |
| ATOM | 2938 | OW | WAT | 609 | 1.173 | 18.896 | 13.815 | 1.00 | 38.79 |
| ATOM | 2939 | OW | WAT | 610 | 21.714 | 21.650 | −7.187 | 1.00 | 30.79 |
| ATOM | 2940 | OW | WAT | 611 | 16.630 | 13.196 | 3.673 | 1.00 | 38.22 |
| ATOM | 2941 | OW | WAT | 612 | 3.332 | 18.798 | 15.551 | 1.00 | 30.36 |
| ATOM | 2942 | OW | WAT | 613 | 11.410 | 46.061 | 15.908 | 1.00 | 30.96 |
| ATOM | 2943 | OW | WAT | 614 | 1.890 | 53.075 | 0.396 | 1.00 | 35.43 |
| ATOM | 2944 | OW | WAT | 615 | 14.858 | 54.460 | −19.563 | 1.00 | 36.48 |
| ATOM | 2945 | OW | WAT | 616 | 27.164 | 22.302 | 9.178 | 1.00 | 28.96 |
| ATOM | 2946 | OW | WAT | 617 | 25.844 | 30.643 | 13.373 | 1.00 | 37.70 |
| ATOM | 2947 | OW | WAT | 618 | −11.773 | 30.992 | 19.536 | 1.00 | 35.27 |
| ATOM | 2948 | OW | WAT | 619 | 20.068 | 54.715 | −26.556 | 1.00 | 30.61 |
| ATOM | 2949 | OW | WAT | 620 | 22.511 | 25.529 | 18.055 | 1.00 | 39.02 |
| ATOM | 2950 | OW | WAT | 621 | 4.762 | 24.578 | 19.147 | 1.00 | 29.70 |
| ATOM | 2951 | OW | WAT | 622 | −5.809 | 31.212 | −7.251 | 1.00 | 38.52 |
| ATOM | 2952 | OW | WAT | 623 | 2.302 | 46.734 | −19.134 | 1.00 | 31.35 |
| ATOM | 2953 | OW | WAT | 624 | −3.267 | 26.845 | −9.657 | 1.00 | 26.55 |
| ATOM | 2954 | OW | WAT | 625 | 20.942 | 19.909 | 15.987 | 1.00 | 30.93 |
| ATOM | 2955 | OW | WAT | 626 | 14.335 | 19.417 | 27.897 | 1.00 | 41.36 |
| ATOM | 2956 | OW | WAT | 627 | −8.960 | 44.991 | 2.623 | 1.00 | 32.33 |
| ATOM | 2957 | OW | WAT | 628 | −2.896 | 18.495 | 3.945 | 1.00 | 33.15 |
| ATOM | 2958 | OW | WAT | 629 | 19.081 | 15.066 | 19.313 | 1.00 | 41.85 |
| ATOM | 2959 | OW | WAT | 630 | 26.583 | 40.965 | −16.598 | 1.00 | 53.36 |
| ATOM | 2960 | OW | WAT | 631 | 9.201 | 30.845 | −29.283 | 1.00 | 26.67 |
| ATOM | 2961 | OW | WAT | 632 | 29.771 | 29.232 | 13.030 | 1.00 | 37.22 |
| ATOM | 2962 | OW | WAT | 633 | −9.063 | 44.258 | 5.485 | 1.00 | 30.64 |
| ATOM | 2963 | OW | WAT | 634 | 36.469 | 24.114 | 2.218 | 1.00 | 34.07 |
| ATOM | 2964 | OW | WAT | 635 | 1.658 | 28.923 | 20.644 | 1.00 | 39.44 |
| ATOM | 2965 | OW | WAT | 636 | −8.637 | 37.196 | −3.769 | 1.00 | 39.41 |
| ATOM | 2966 | OW | WAT | 637 | 9.491 | 43.672 | 18.552 | 1.00 | 34.67 |
| ATOM | 2967 | OW | WAT | 638 | 38.446 | 24.948 | 5.405 | 1.00 | 37.88 |
| ATOM | 2968 | OW | WAT | 639 | 16.362 | 21.306 | −12.437 | 1.00 | 35.82 |
| ATOM | 2969 | OW | WAT | 640 | 11.407 | 51.004 | −0.072 | 1.00 | 31.55 |
| ATOM | 2970 | OW | WAT | 641 | 38.229 | 24.335 | 8.085 | 1.00 | 39.89 |
| ATOM | 2971 | OW | WAT | 642 | 21.655 | 26.806 | 22.131 | 1.00 | 32.81 |
| ATOM | 2972 | OW | WAT | 643 | 16.387 | 22.635 | 23.545 | 1.00 | 35.28 |
| ATOM | 2973 | OW | WAT | 644 | −12.122 | 42.861 | −8.757 | 1.00 | 43.21 |
| ATOM | 2974 | OW | WAT | 645 | −1.768 | 30.006 | 19.108 | 1.00 | 39.03 |
| ATOM | 2975 | OW | WAT | 646 | 31.231 | 36.441 | −7.964 | 1.00 | 40.74 |
| ATOM | 2976 | OW | WAT | 647 | −9.784 | 38.920 | 18.620 | 1.00 | 37.25 |
| ATOM | 2977 | OW | WAT | 648 | −5.666 | 31.659 | 21.328 | 1.00 | 32.24 |
| ATOM | 2978 | OW | WAT | 649 | −2.584 | 54.436 | 0.499 | 1.00 | 42.36 |
| ATOM | 2979 | OW | WAT | 650 | 9.314 | 15.276 | 13.185 | 1.00 | 41.13 |
| ATOM | 2980 | OW | WAT | 651 | 20.108 | 12.329 | 9.346 | 1.00 | 30.57 |
| ATOM | 2981 | OW | WAT | 652 | 28.719 | 20.042 | 8.674 | 1.00 | 30.27 |
| ATOM | 2982 | OW | WAT | 653 | 27.567 | 35.432 | 11.915 | 1.00 | 34.47 |
| ATOM | 2983 | OW | WAT | 654 | 20.822 | 18.155 | 14.214 | 1.00 | 29.75 |
| ATOM | 2984 | OW | WAT | 655 | −1.395 | 25.107 | 7.194 | 1.00 | 42.08 |
| END | | | | | | | | | |

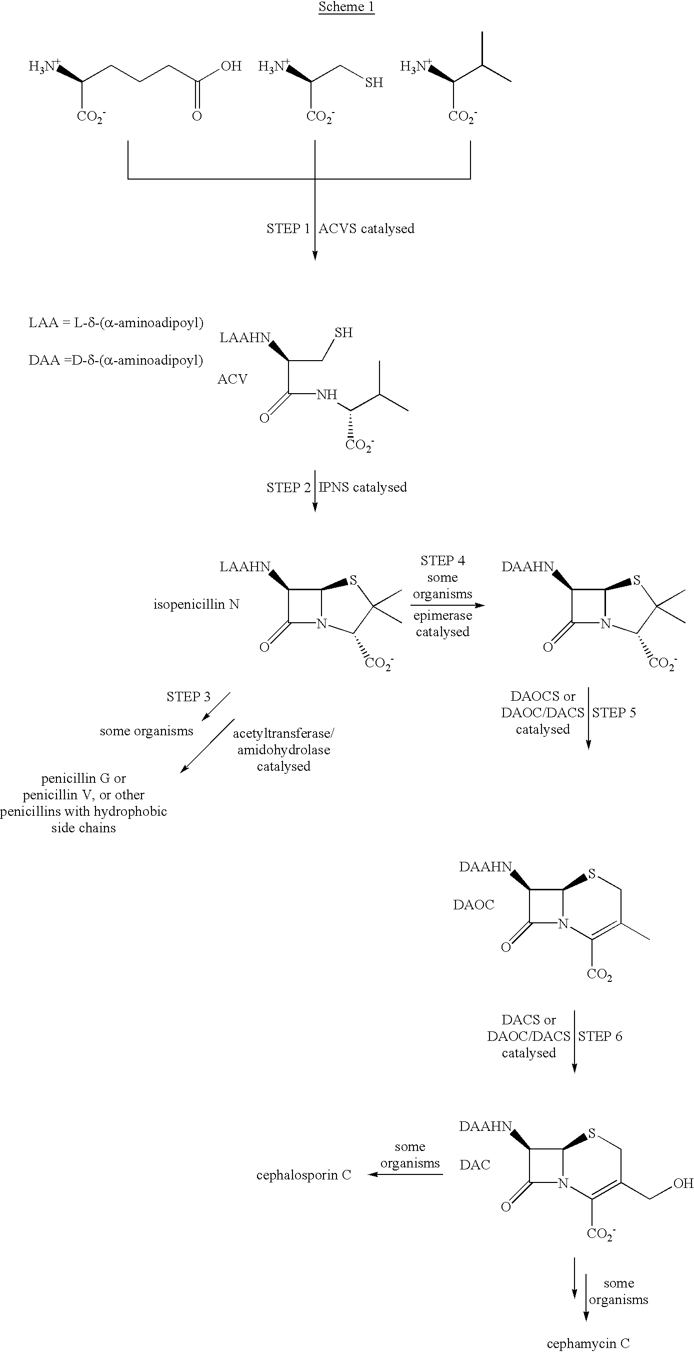

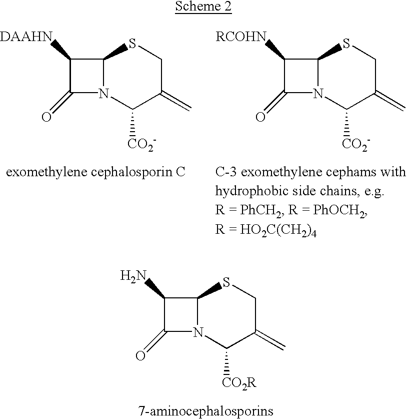

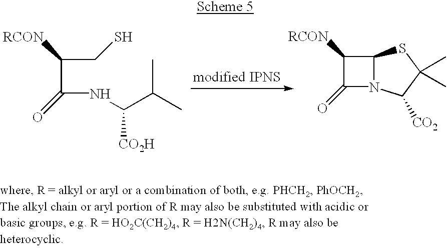

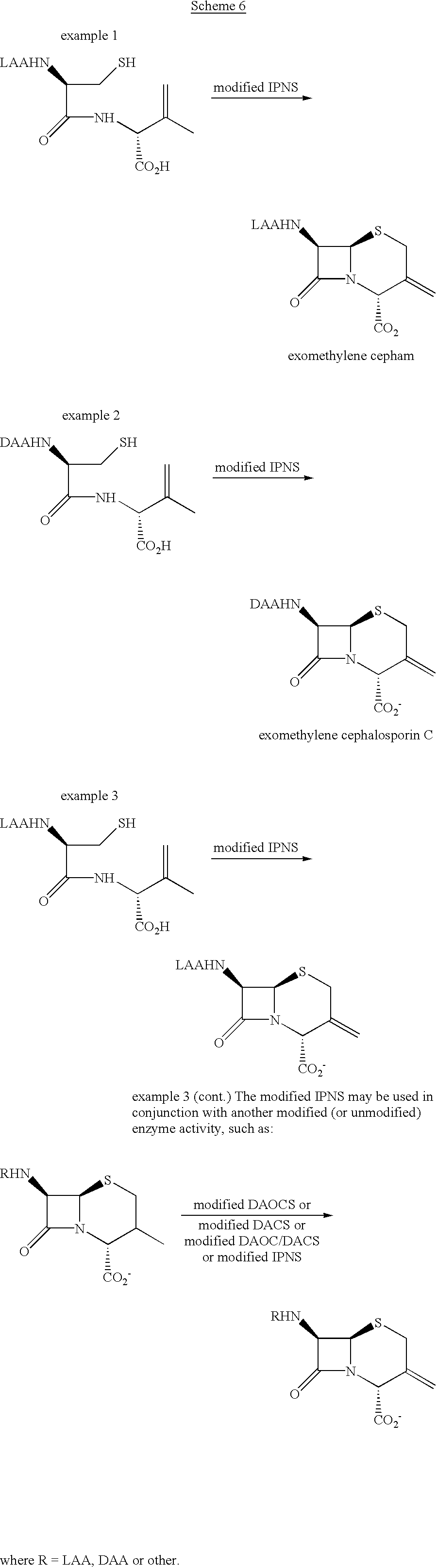

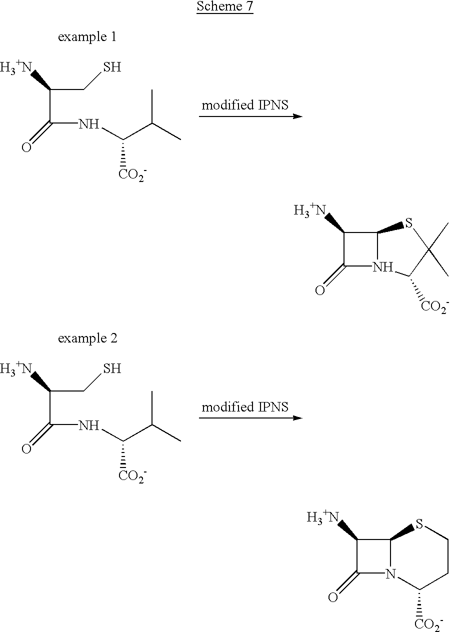

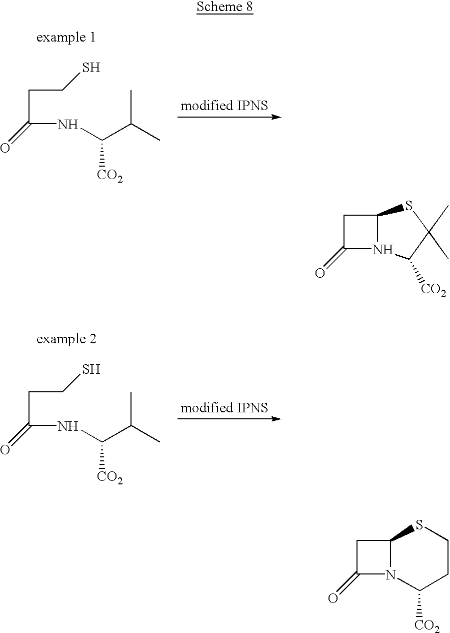

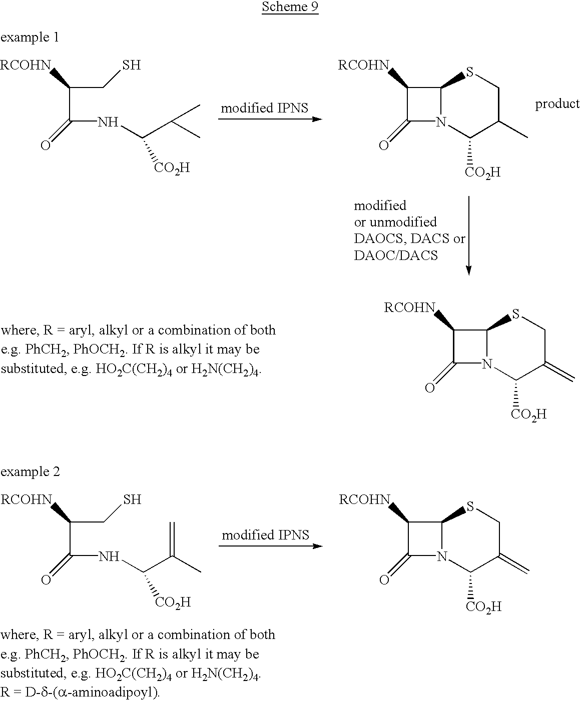

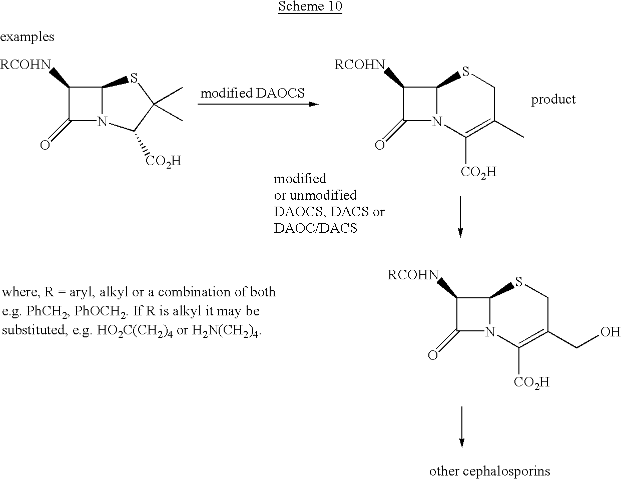

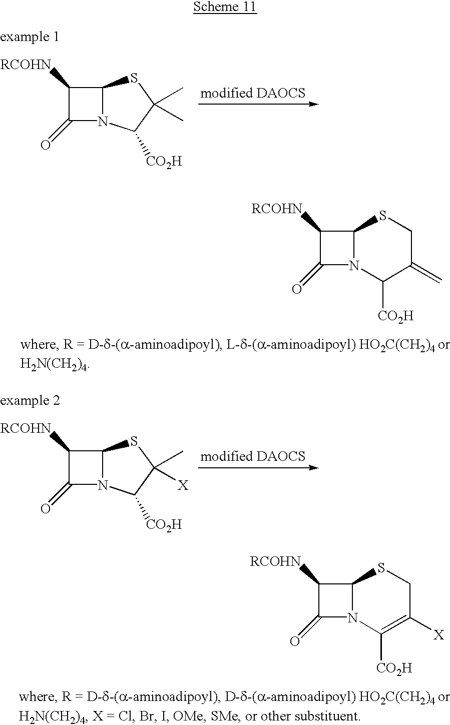

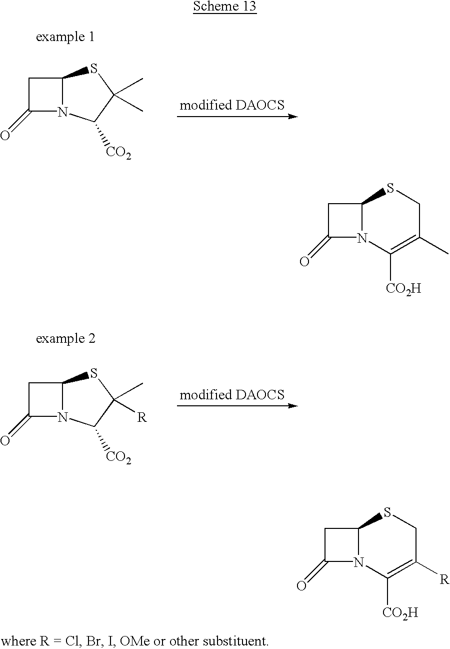

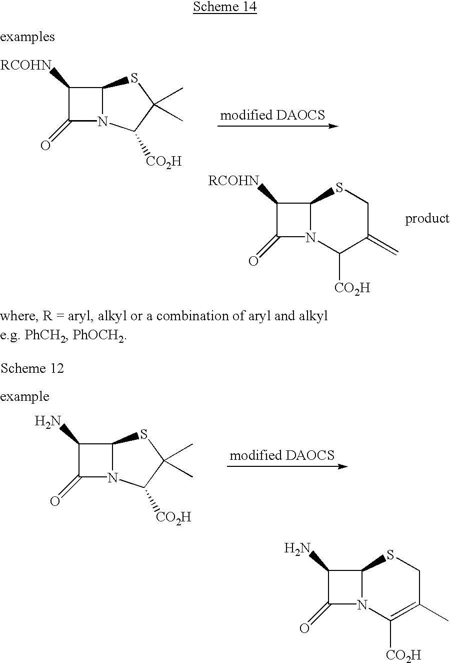

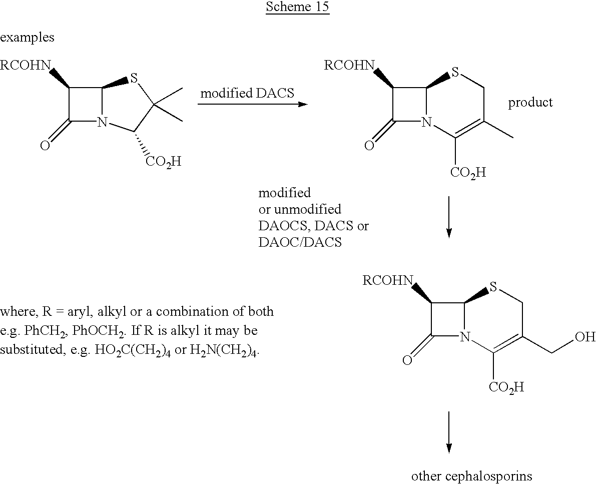

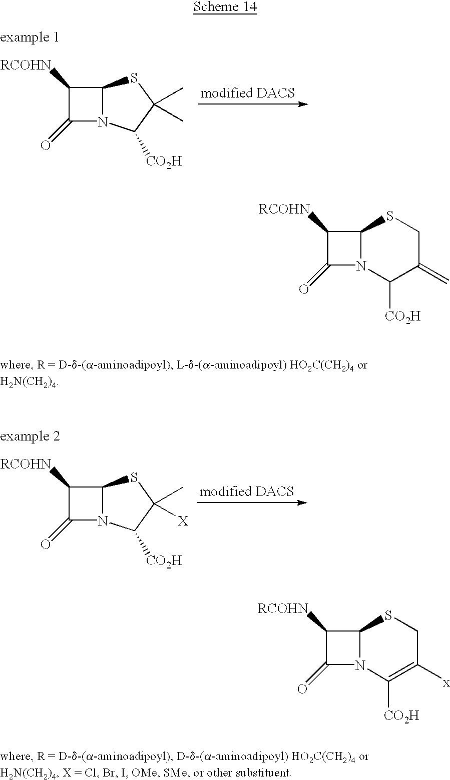

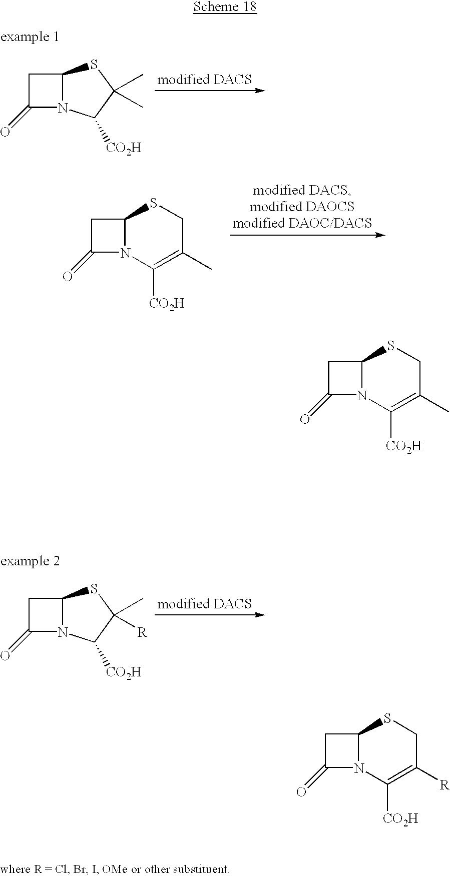

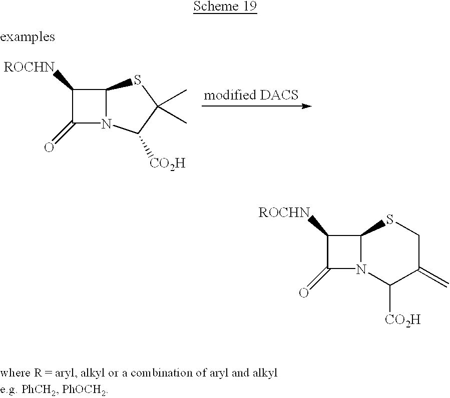

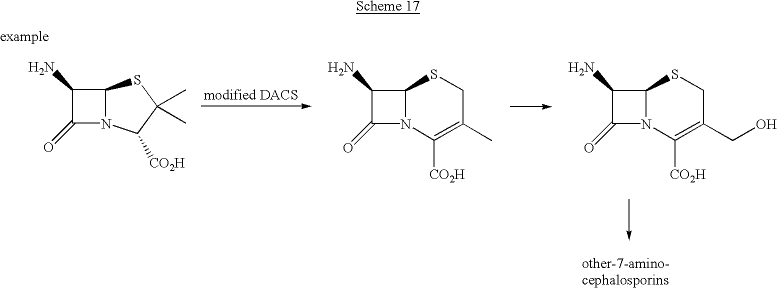

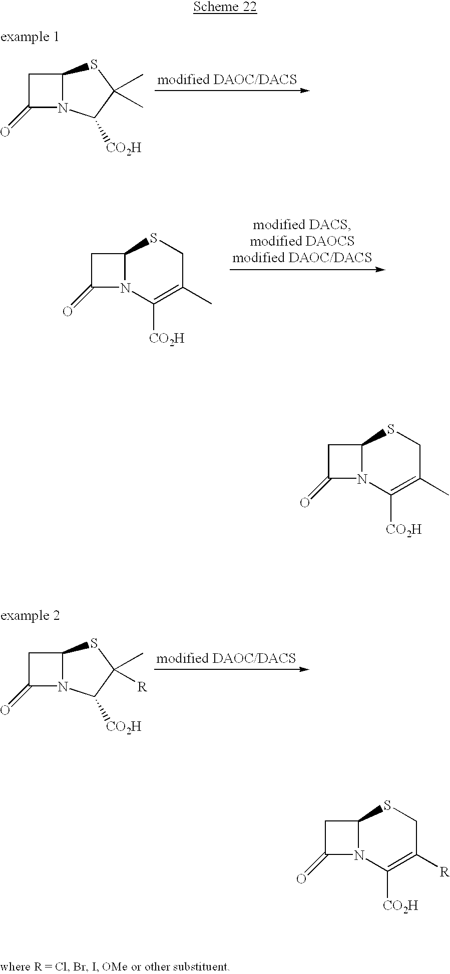

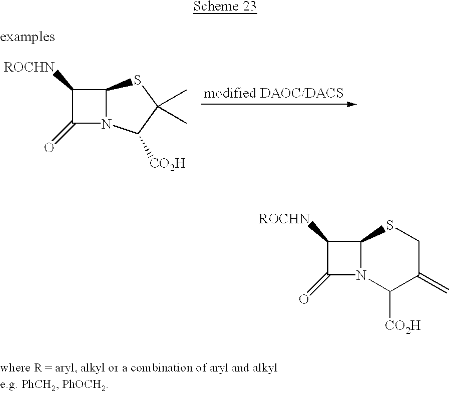

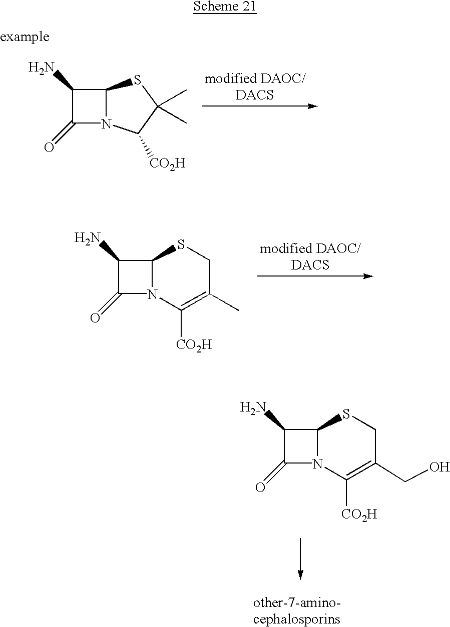

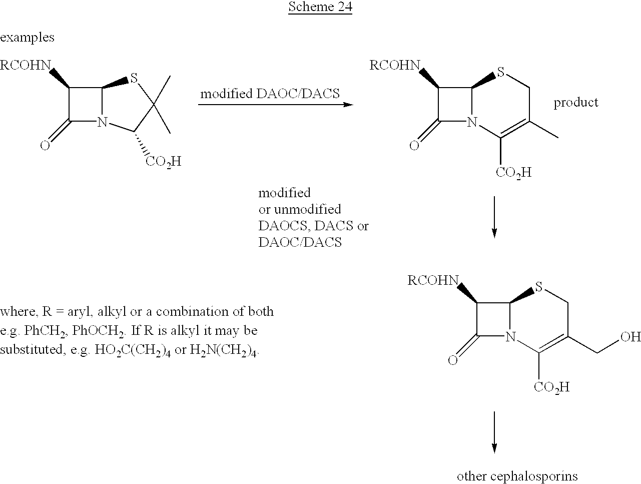

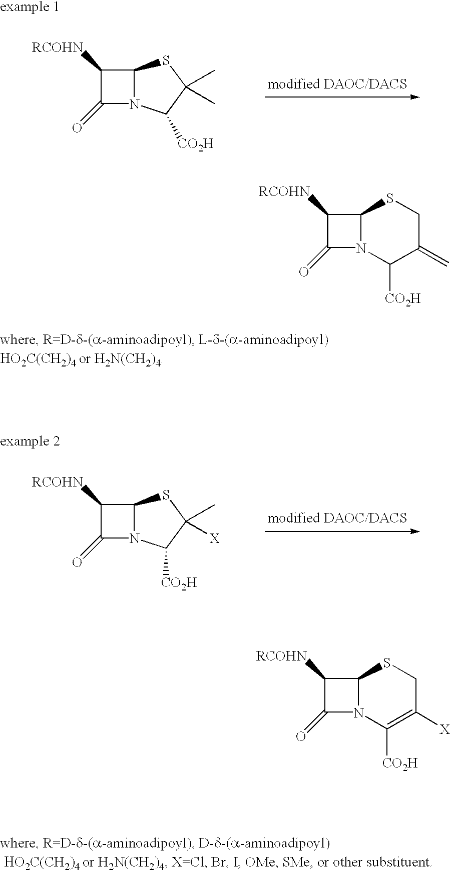

1. Isopenicillin N synthase (IPNS) in the form of: a complex with Fe and its substrate, said complex having a structure substantially designated by the X-ray co-ordinates in Table 3.

2. The IPNS complex of claim 1, wherein the substrate is L-δ-α-aminoadipoyl-L-cysteinyl-D-valine (ACV).

3. The IPNS complex of claim 1 wherein the substrate is an analogue of ACV selected from AC glycine, Ac aminobutyrate, AC alanine and AC propargylglycine.

4. Use of the three dimensional structure of a first enzyme selected from IPNS, DAOCS, DACS, DAOC/DACS and other related enzymes of the penicillin and cephalosporin biosynthesis pathway, for the modification of a second enzyme selected from IPNS, DAOCS, DACS, DAOC/DACS and other related enzymes of the penicillin and cephalosporin biosynthesis pathway.

5. Use as claimed in claim 4, wherein the second enzyme is modified: to accept unnatural substrates for the preparation of antibacterial materials or intermediate for the production of pharmaceutical products; or to produce unnatural products or improve the production of natural products.

6. An enzyme having significant (as herein defined) sequence similarity to IPNS, wherein at least one of the following amino acid residues is modified:
N287; R87; A88; Y189; S183; Y91; F285; Q330; T331; V185; L106; C104; V217; L324; L317; I325; L321; S210.

7. An enzyme having significant (as herein defined) sequence similarity to IPNS, wherein at least one of the following amino acid residues is modified:
V272; L231; L223; P283; T221; F211, F285; Q330; I187; V185; Y189; R279; S281, N230; Q225; N252; S210.

8. A gene which codes for the enzyme of claim 6 or claim 7.

9. A micro-organism, containing the gene of claim 8 and which is capable of expressing the gene under fermentation conditions.

10. Use of the microorganism of claim 9 for making a bicyclic β-lactam of the penicillin or cephalosporin (Including cephams) families.

11. Use of the enzyme of claim 6 or claim 7 for the preparation in vitro of a bicyclic β-lactam of the penicillin or cephalosporin families.

12. In a method for the preparation of an enzyme, selected from IPNS, DAOCS, DACS, DAOC/DACS and sequence-related enzymes, in crystalline form for X-ray diffraction studies, the improvement which consists in maintaining the enzyme under anaerobic conditions with dioxygen substantially absent.

13. A method which comprises using the three dimensional structure of a first enzyme selected from IPNS DAOCS, DACS, DAOC/DACS and other related enzymes of the penicillin and cephalosporin biosynthesis pathway, for determining or predicting the structure of a second enzyme which is structurally related to the first enzyme but is not active in the penicillin or cephalosporin biosynthesis pathway, and using the structural information so obtained for modifying the second enzyme or for designing an inhibitor for the second enzyme.

14. Use of the enzyme of claim 6 or claim 7 to convert a dipeptide to a 6- aminopenicillin or other bicyclic β-lactam.

15. Use as claimed in claim 14, wherein the dipeptide has been produced by use of a peptide synthetase enzyme such as L-δ-α-aminoadipoyl-L-cysteinyl-D-valine (ACV) synthetase optionally modified to optimise dipeptide production.
